SEQUENCE LISTING (646 sequences)

SEQ ID NO: 1 - DNA, 72 bp, Homo sapiens
gggggtatagctcagtggtagagcgcgtgcttagcatgcacgaggtcctgggttcgatccccagtacctcca

SEQ ID NO: 2 - DNA, 73 bp, Homo sapiens
ggggaattagctcaagtggtagagcgcttgcttagcacgcaagaggtagtgggatcgatgcccacattctcca

SEQ ID NO: 3 - DNA, 73 bp, Homo sapiens
ggggaattagctcaaatggtagagcgctcgcttagcatgcgagaggtagcgggatcgatgcccgcattctcca

SEQ ID NO: 4 - DNA, 73 bp, Homo sapiens
ggggaattagctcaagtggtagagcgcttgcttagcatgcaagaggtagtgggatcgatgcccacattctcca

SEQ ID NO: 5 - DNA, 73 bp, Homo sapiens
ggggaattagctcaagcggtagagcgcttgcttagcatgcaagaggtagtgggatcgatgcccacattctcca

SEQ ID NO: 6 - DNA, 73 bp, Homo sapiens
ggggaattagctcaagtggtagagcgcttgcttagcatgcaagaggtagtgggatcaatgcccacattctcca

SEQ ID NO: 7 - DNA, 73 bp, Homo sapiens
ggggaattagctcaagtggtagagcgctcgcttagcatgcgagaggtagtgggatcgatgcccgcattctcca

SEQ ID NO: 8 - DNA, 73 bp, Homo sapiens
ggggaattagcccaagtggtagagcgcttgcttagcatgcaagaggtagtgggatcgatgcccacattctcca

SEQ ID NO: 9 - DNA, 72 bp, Homo sapiens
gggggtgtagctcagtggtagagcgcgtgcttagcatgcacgaggccccgggttcaatccccggcacctcca

SEQ ID NO: 10 - DNA, 72 bp, Homo sapiens
gggggtgtagctcagtggtagagcgcgtgcttagcatgtacgaggtcccgggttcaatccccggcacctcca

SEQ ID NO: 11 - DNA, 72 bp, Homo sapiens
ggggatgtagctcagtggtagagcgcatgcttagcatgcatgaggtcccgggttcgatccccagcatctcca

SEQ ID NO: 12 - DNA, 72 bp, Homo sapiens
gggggtgtagctcagtggtagagcgcgtgcttagcatgcacgaggccctgggttcaatccccagcacctcca

SEQ ID NO: 13 - DNA, 72 bp, Homo sapiens
gggggtatagctcagcggtagagcgcgtgcttagcatgcacgaggtcctgggttcaatccccaatacctcca

SEQ ID NO: 14 - DNA, 72 bp, Homo sapiens
gggggtgtagctcagtggtagagcgcgtgcttagcatgcacgaggccccgggttcaatccctggcacctcca

SEQ ID NO: 15 - DNA, 73 bp, Homo sapiens
gggggattagctcaaatggtagagcgctcgcttagcatgcgagaggtagcgggatcgatgcccgcatcctcca

SEQ ID NO: 16 - DNA, 73 bp, Homo sapiens
ggggaattagctcaggcggtagagcgctcgcttagcatgcgagaggtagcgggatcgacgcccgcattctcca

SEQ ID NO: 17 - DNA, 72 bp, Homo sapiens
ggggatgtagctcagtggtagagcgcatgcttcgcatgtatgaggtcccgggttcgatccccggcatctcca

SEQ ID NO: 18 - DNA, 72 bp, Homo sapiens
ggggatgtagctcagtggtagagcgcatgcttcgcatgtatgaggccccgggttcgatccccggcatctcca

SEQ ID NO: 19 - DNA, 72 bp, Homo sapiens
ggggatgtagctcagtggtagagcgcgcgcttcgcatgtgtgaggtcccgggttcaatccccggcatctcca

SEQ ID NO: 20 - DNA, 72 bp, Homo sapiens
gggggtgtagctcagtggtagagcgcgtgcttcgcatgtacgaggccccgggttcgacccccggctcctcca

SEQ ID NO: 21 - DNA, 72 bp, Homo sapiens
gggggtgtagctcagtggtagagcgcatgctttgcatgtatgaggtcccgggttcgatccccggcacctcca

SEQ ID NO: 22 - DNA, 72 bp, Homo sapiens
ggggatgtagctcagtggtagagcgcatgctttgcatgtatgaggtcccgggttcgatccccggcatctcca

SEQ ID NO: 23 - DNA, 72 bp, Homo sapiens
ggggatgtagctcagtggtagagcgcatgctttgcatgtatgaggccccgggttcgatccccggcatctcca

SEQ ID NO: 24 - DNA, 72 bp, Homo sapiens
ggggatgtagctcagtggtagagcgcatgctttgcacgtatgaggccccgggttcaatccccggcatctcca

SEQ ID NO: 25 - DNA, 72 bp, Homo sapiens
gggggtgtagctcagtggtagagcgcatgctttgcatgtatgaggcctcgggttcgatccccgacacctcca

SEQ ID NO: 26 - DNA, 72 bp, Homo sapiens
gggggtgtagctcagtggtagagcacatgctttgcatgtgtgaggccccgggttcgatccccggcacctcca

SEQ ID NO: 27 - DNA, 71 bp, Homo sapiens
gggggtgtagctcagtggtagagcgcatgctttgcatgtatgaggcctcggttcgatccccgacacctcca

SEQ ID NO: 28 - DNA, 73 bp, Homo sapiens
gggccagtggcgcaatggataacgcgtctgactacggatcagaagattccaggttcgactcctggctggctcg

SEQ ID NO: 29 - DNA, 73 bp, Homo sapiens
gggccagtggcgcaatggataacgcgtctgactacggatcagaagattctaggttcgactcctggctggctcg

SEQ ID NO: 30 - DNA, 73 bp, Homo sapiens
ggccgcgtggcctaatggataaggcgtctgattccggatcagaagattgagggttcgagtcccttcgtggtcg

SEQ ID NO: 31 - DNA, 73 bp, Homo sapiens
gacccagtggcctaatggataaggcatcagcctccggagctggggattgtgggttcgagtcccatctgggtcg

SEQ ID NO: 32 - DNA, 73 bp, Homo sapiens
gccccagtggcctaatggataaggcactggcctcctaagccagggattgtgggttcgagtcccacctggggta

SEQ ID NO: 33 - DNA, 73 bp, Homo sapiens
gccccagtggcctaatggataaggcactggcctcctaagccagggattgtgggttcgagtcccacctggggtg

SEQ ID NO: 34 - DNA, 73 bp, Homo sapiens
gccccggtggcctaatggataaggcattggcctcctaagccagggattgtgggttcgagtcccacccggggta

SEQ ID NO: 35 - DNA, 73 bp, Homo sapiens
gccccagtggcctaatggataaggcattggcctcctaagccagggattgtgggttcgagtcccatctggggtg

SEQ ID NO: 36 - DNA, 73 bp, Homo sapiens
gccccagtggcctgatggataaggtactggcctcctaagccagggattgtgggttcgagttccacctggggta

SEQ ID NO: 37 - DNA, 73 bp, Homo sapiens
ggccgcgtggcctaatggataaggcgtctgacttcggatcagaagattgcaggttcgagtcctgccgcggtcg

SEQ ID NO: 38 - DNA, 73 bp, Homo sapiens
gaccacgtggcctaatggataaggcgtctgacttcggatcagaagattgagggttcgaatccctccgtggtta

SEQ ID NO: 39 - DNA, 73 bp, Homo sapiens
gaccgcgtggcctaatggataaggcgtctgacttcggatcagaagattgagggttcgagtcccttcgtggtcg

SEQ ID NO: 40 - DNA, 73 bp, Homo sapiens
gaccacgtggcctaatggataaggcgtctgacttcggatcagaagattgagggttcgaatcccttcgtggtta

SEQ ID NO: 41 - DNA, 73 bp, Homo sapiens
gaccacgtggcctaatggataaggcgtctgacttcggatcagaagattgagggttcgaatcccttcgtggttg

SEQ ID NO: 42 - DNA, 73 bp, Homo sapiens
ggccgtgtggcctaatggataaggcgtctgacttcggatcaaaagattgcaggtttgagttctgccacggtcg

SEQ ID NO: 43 - DNA, 85 bp, Homo sapiens
ggctccgtggcgcaatggatagcgcattggacttctagaggctgaaggcattcaaaggttccgggttcgagtcccggcggagtcg

SEQ ID NO: 44 - DNA, 88 bp, Homo sapiens
ggctctgtggcgcaatggatagcgcattggacttctagtgacgaatagagcaattcaaaggttgtgggttcgaatcccaccagagtcg

SEQ ID NO: 45 - DNA, 91 bp, Homo sapiens
ggctctgtggcgcaatggatagcgcattggacttctagctgagcctagtgtggtcattcaaaggttgtgggttcgagtcccaccagagtcg

SEQ ID NO: 46 - DNA, 86 bp, Homo sapiens
ggctctgtggcgcaatggatagcgcattggacttctagatagttagagaaattcaaaggttgtgggttcgagtcccaccagagtcg

SEQ ID NO: 47 - DNA, 74 bp, Homo sapiens
gtctctgtggcgcaatggacgagcgcgctggacttctaatccagaggttccgggttcgagtcccggcagagatg

SEQ ID NO: 48 - DNA, 87 bp, Homo sapiens
ggctctgtggcgcaatggatagcgcattggacttctagcctaaatcaagagattcaaaggttgcgggttcgagtccctccagagtcg

SEQ ID NO: 49 - DNA, 74 bp, Homo sapiens
gtctctgtggcgcaatcggttagcgcgttcggctgttaaccgaaaggttggtggttcgatcccacccagggacg

SEQ ID NO: 50 - DNA, 74 bp, Homo sapiens
gtctctgtggcgcaatcggctagcgcgtttggctgttaactaaaaggttggcggttcgaacccacccagaggcg

SEQ ID NO: 51 - DNA, 74 bp, Homo sapiens
gtctctgtggtgcaatcggttagcgcgttccgctgttaaccgaaagcttggtggttcgagcccacccagggatg

SEQ ID NO: 52 - DNA, 74 bp, Homo sapiens
gtctctgtggcgcaatcggctagcgcgtttggctgttaactaaaaagttggtggttcgaacacacccagaggcg

SEQ ID NO: 53 - DNA, 74 bp, Homo sapiens
gtctctgtggcgcaatcggttagcgcgttcggctgttaaccgaaaggttggtggttcgagcccacccagggacg

SEQ ID NO: 54 - DNA, 74 bp, Homo sapiens
gtctctgtggcgcaatcggttagcgcattcggctgttaaccgaaaggttggtggttcgagcccacccagggacg

SEQ ID NO: 55 - DNA, 74 bp, Homo sapiens
gtctctgtggcgcaatcggttagcgcgttcggctgttaaccgaaagattggtggttcgagcccacccagggacg

SEQ ID NO: 56 - DNA, 74 bp, Homo sapiens
gtctctgtggcgcaatcggttagcgcgttcggctgttaactgaaaggttggtggttcgagcccacccagggacg

SEQ ID NO: 57 - DNA, 74 bp, Homo sapiens
gtctctgtggcgcaatgggttagcgcgttcggctgttaaccgaaaggttggtggttcgagcccatccagggacg

SEQ ID NO: 58 - DNA, 74 bp, Homo sapiens
gtctctgtggcgtagtcggttagcgcgttcggctgttaaccgaaaagttggtggttcgagcccacccaggaacg

SEQ ID NO: 59 - DNA, 74 bp, Homo sapiens
gtctctgtggcgcaatcggctagcgcgtttggctgttaactaaaaggttggtggttcgaacccacccagaggcg

SEQ ID NO: 60 - DNA, 74 bp, Homo sapiens
gtctctgtggcgcaatcggttagcgcgttcggctgttaactgaaaggttagtggttcgagcccacccggggacg

SEQ ID NO: 61 - DNA, 72 bp, Homo sapiens
tcctcgttagtatagtggttagtatccccgcctgtcacgcgggagaccggggttcaattccccgacggggag

SEQ ID NO: 62 - DNA, 72 bp, Homo sapiens
tcctcgttagtatagtggtgagtatccccgcctgtcacgcgggagaccggggttcgattccccgacggggag

SEQ ID NO: 63 - DNA, 72 bp, Homo sapiens
tcctcgttagtatagtggtgagtgtccccgtctgtcacgcgggagaccggggttcgattccccgacggggag

SEQ ID NO: 64 - DNA, 72 bp, Homo sapiens
gggggcatagctcagtggtagagcatttgactgcagatcaagaggtccctggttcaaatccaggtgccccct

SEQ ID NO: 65 - DNA, 72 bp, Homo sapiens
gggggtatagctcaggggtagagcatttgactgcagatcaagaggtccctggttcaaatccaggtgcccccc

SEQ ID NO: 66 - DNA, 72 bp, Homo sapiens
gggggtatagcttagcggtagagcatttgactgcagatcaagaggtccccggttcaaatccgggtgccccct

SEQ ID NO: 67 - DNA, 72 bp, Homo sapiens
gggggtatagcttaggggtagagcatttgactgcagatcaaaaggtccctggttcaaatccaggtgcccctt

SEQ ID NO: 68 - DNA, 72 bp, Homo sapiens
gggggtatagctcaggggtagagcatttgactgcagatcaagaggtccccagttcaaatctgggtgccccct

SEQ ID NO: 69 - DNA, 72 bp, Homo sapiens
gggggtatagctcaggggtagagcatttgactgcagatcaagaagtccccggttcaaatccgggtgccccct

SEQ ID NO: 70 - DNA, 72 bp, Homo sapiens
gggggtatagctcaggggtagagcatttgactgcagatcaagaggtctctggttcaaatccaggtgccccct

SEQ ID NO: 71 - DNA, 72 bp, Homo sapiens
gggggtatagctcaggggtagagcacttgactgcagatcaagaagtccttggttcaaatccaggtgccccct

SEQ ID NO: 72 - DNA, 72 bp, Homo sapiens
ggggatatagctcaggggtagagcatttgactgcagatcaagaggtccccggttcaaatccgggtgcccccc

SEQ ID NO: 73 - DNA, 72 bp, Homo sapiens
gggggtatagttcaggggtagagcatttgactgcagatcaagaggtccctggttcaaatccaggtgccccct

SEQ ID NO: 74 - DNA, 72 bp, Homo sapiens
gggggtatagctcaggggtagagcatttgactgcaaatcaagaggtccctgattcaaatccaggtgccccct

SEQ ID NO: 75 - DNA, 72 bp, Homo sapiens
gggggtatagctcagtggtagagcatttgactgcagatcaagaggtccccggttcaaatccgggtgccccct

SEQ ID NO: 76 - DNA, 72 bp, Homo sapiens
gggcgtatagctcaggggtagagcatttgactgcagatcaagaggtccccagttcaaatctgggtgccccct

SEQ ID NO: 77 - DNA, 72 bp, Homo sapiens
gggggtatagctcacaggtagagcatttgactgcagatcaagaggtccccggttcaaatctgggtgccccct

SEQ ID NO: 78 - DNA, 70 bp, Homo sapiens
gggcgtatagctcaggggtagagcatttgactgcagatcaagaggtccccagttcaaatctgggtgccca

SEQ ID NO: 79 - DNA, 72 bp, Homo sapiens
gggggtatagctcacaggtagagcatttgactgcagatcaagaggtccccggttcaaatccggttactccct

SEQ ID NO: 80 - DNA, 72 bp, Homo sapiens
gggggtatagctcaggggtagagcacttgactgcagatcaagaggtccctggttcaaatccaggtgccccct

SEQ ID NO: 81 - DNA, 72 bp, Homo sapiens
gggggtatagctcagtggtagagcatttgactgcagatcaagaggtccctggttcaaatccgggtgccccct

SEQ ID NO: 82 - DNA, 73 bp, Homo sapiens
gggggtatagctcagtgggtagagcatttgactgcagatcaagaggtccccggttcaaatccgggtgccccct

SEQ ID NO: 83 - DNA, 72 bp, Homo sapiens
gggggtgtagctcagtggtagagcatttgactgcagatcaagaggtccctggttcaaatccaggtgccccct

SEQ ID NO: 84 - DNA, 73 bp, Homo sapiens
gggggtatagctcaggtggtagagcatttgactgcagatcaagaggtccccggttcaaatccgggtgccccct

SEQ ID NO: 85 - DNA, 72 bp, Homo sapiens
gggggtatagctcaggggtagagcatttgactgcagatcaagaggtccccggttcaaatccgggtgccccct

SEQ ID NO: 86 - DNA, 72 bp, Homo sapiens
gggggtatagctcaggggtagagcatttgactgcagatcaagaggtccctggttcaaatccaggtgccccct

SEQ ID NO: 87 - DNA, 72 bp, Homo sapiens
ggttccatggtgtaatggttagcactctggactctgaatccagcgatccgagttcaaatctcggtggaacct

SEQ ID NO: 88 - DNA, 72 bp, Homo sapiens
ggttccatggtgtaatggttagcactctggactctgaatccagcgatccgagttcaagtctcggtggaacct

SEQ ID NO: 89 - DNA, 72 bp, Homo sapiens
ggttccatggtgtaatggtgagcactctggactctgaatccagcgatccgagttcgagtctcggtggaacct

SEQ ID NO: 90 - DNA, 72 bp, Homo sapiens
ggttccatggtgtaatggtaagcactctggactctgaatccagcgatccgagttcgagtctcggtggaacct

SEQ ID NO: 91 - DNA, 72 bp, Homo sapiens
ggttccatggtgtaatggttagcactctggactctgaatccggtaatccgagttcaaatctcggtggaacct

SEQ ID NO: 92 - DNA, 72 bp, Homo sapiens
ggccccatggtgtaatggtcagcactctggactctgaatccagcgatccgagttcaaatctcggtgggaccc

SEQ ID NO: 93 - DNA, 72 bp, Homo sapiens
ggttccatggtgtaatggtaagcactctggactctgaatccagccatctgagttcgagtctctgtggaacct

SEQ ID NO: 94 - DNA, 72 bp, Homo sapiens
ggtcccatggtgtaatggttagcactctggactttgaatccagcgatccgagttcaaatctcggtgggacct

SEQ ID NO: 95 - DNA, 72 bp, Homo sapiens
ggtcccatggtgtaatggttagcactctggactttgaatccagcaatccgagttcgaatctcggtgggacct

SEQ ID NO: 96 - DNA, 72 bp, Homo sapiens
ggccccatggtgtaatggttagcactctggactttgaatccagcgatccgagttcaaatctcggtgggacct

SEQ ID NO: 97 - DNA, 72 bp, Homo sapiens
ggtcccatggtgtaatggttagcactctgggctttgaatccagcaatccgagttcgaatcttggtgggacct

SEQ ID NO: 98 - DNA, 72 bp, Homo sapiens
tccctggtggtctagtggttaggattcggcgctctcaccgccgcggcccgggttcgattcccggtcagggaa

SEQ ID NO: 99 - DNA, 72 bp, Homo sapiens
tccctggtggtctagtggttaggattcggcgctctcaccgccgcggcccgggttcgattcccggtcaggaaa

SEQ ID NO: 100 - DNA, 72 bp, Homo sapiens
tcccatatggtctagcggttaggattcctggttttcacccaggtggcccgggttcgactcccggtatgggaa

SEQ ID NO: 101 - DNA, 72 bp, Homo sapiens
tcccacatggtctagcggttaggattcctggttttcacccaggcggcccgggttcgactcccggtgtgggaa

SEQ ID NO: 102 - DNA, 72 bp, Homo sapiens
tccctggtggtctagtggctaggattcggcgctttcaccgccgcggcccgggttcgattcccggccagggaa

SEQ ID NO: 103 - DNA, 72 bp, Homo sapiens
tccctggtggtctagtggctaggattcggcgctttcaccgccgcggcccgggttcgattcccggtcagggaa

SEQ ID NO: 104 - DNA, 71 bp, Homo sapiens
gcattggtggttcagtggtagaattctcgcctcccacgcgggagacccgggttcaattcccggccaatgca

SEQ ID NO: 105 - DNA, 71 bp, Homo sapiens
gcgccgctggtgtagtggtatcatgcaagattcccattcttgcgacccgggttcgattcccgggcggcgca

SEQ ID NO: 106 - DNA, 71 bp, Homo sapiens
gcattggtggttcaatggtagaattctcgcctcccacgcaggagacccaggttcgattcctggccaatgca

SEQ ID NO: 107 - DNA, 71 bp, Homo sapiens
gcatgggtggttcagtggtagaattctcgcctgccacgcgggaggcccgggttcgattcccggcccatgca

SEQ ID NO: 108 - DNA, 71 bp, Homo sapiens
gcattggtggttcagtggtagaattctcgcctgccacgcgggaggcccgggttcgattcccggccaatgca

SEQ ID NO: 109 - DNA, 71 bp, Homo sapiens
gcattggtggttcagtggtagaattctcgcctgccacgcgggaggcccgggtttgattcccggccagtgca

SEQ ID NO: 110 - DNA, 71 bp, Homo sapiens
gcataggtggttcagtggtagaattcttgcctgccacgcaggaggcccaggtttgattcctggcccatgca

SEQ ID NO: 111 - DNA, 71 bp, Homo sapiens
gcattggtggttcagtggtagaattctcgcctgccatgcgggcggccgggcttcgattcctggccaatgca

SEQ ID NO: 112 - DNA, 72 bp, Homo sapiens
gcgttggtggtatagtggttagcatagctgccttccaagcagttgacccgggttcgattcccggccaacgca

SEQ ID NO: 113 - DNA, 72 bp, Homo sapiens
gcgttggtggtatagtggtgagcatagctgccttccaagcagttgacccgggttcgattcccggccaacgca

SEQ ID NO: 114 - DNA, 72 bp, Homo sapiens
gcgttggtggtatagtggtaagcatagctgccttccaagcagttgacccgggttcgattcccggccaacgca

SEQ ID NO: 115 - DNA, 72 bp, Homo sapiens
gcgttggtggtatagtggtgagcatagttgccttccaagcagttgacccgggctcgattcccgcccaacgca

SEQ ID NO: 116 - DNA, 72 bp, Homo sapiens
gccgtgatcgtatagtggttagtactctgcgttgtggccgcagcaacctcggttcgaatccgagtcacggca

SEQ ID NO: 117 - DNA, 72 bp, Homo sapiens
gccatgatcgtatagtggttagtactctgcgctgtggccgcagcaacctcggttcgaatccgagtcacggca

SEQ ID NO: 118 - DNA, 74 bp, Homo sapiens
ggccggttagctcagttggttagagcgtggcgctaataacgccaaggtcgcgggttcgatccccgtacgggcca

SEQ ID NO: 119 - DNA, 74 bp, Homo sapiens
ggccggttagctcagttggttagagcgtggtgctaataacgccaaggtcgcgggttcgatccccgtactggcca

SEQ ID NO: 120 - DNA, 74 bp, Homo sapiens
ggctggttagctcagttggttagagcgtggtgctaataacgccaaggtcgcgggttcgatccccgtactggcca

SEQ ID NO: 121 - DNA, 74 bp, Homo sapiens
ggccggttagctcagttggttagagcgtggtgctaataacgccaaggtcgcgggttcgaaccccgtacgggcca

SEQ ID NO: 122 - DNA, 74 bp, Homo sapiens
ggccggttagctcagttggttagagcgtggtgctaataacgccaaggtcgcgggttcgatccccgtacgggcca

SEQ ID NO: 123 - DNA, 74 bp, Homo sapiens
ggccggttagctcagttggttagagcgtggtgctaataacgctaaggtcgcgggttcgatccccgtactggcca

SEQ ID NO: 124 - DNA, 74 bp, Homo sapiens
ggccggttagctcagttggtcagagcgtggtgctaataacgccaaggtcgcgggttcgatccccgtacgggcca

SEQ ID NO: 125 - DNA, 74 bp, Homo sapiens
ggccggttagctcagtcggctagagcgtggtgctaataacgccaaggtcgcgggttcgatccccgtacgggcca

SEQ ID NO: 126 - DNA, 74 bp, Homo sapiens
ggctggttagttcagttggttagagcgtggtgctaataacgccaaggtcgtgggttcgatccccatatcggcca

SEQ ID NO: 127 - DNA, 74 bp, Homo sapiens
ggccggttagctcagttggtaagagcgtggtgctgataacaccaaggtcgcgggctcgactcccgcaccggcca

SEQ ID NO: 128 - DNA, 93 bp, Homo sapiens
gctccagtggcgcaatcggttagcgcgcggtacttatatgacagtgcgagcggagcaatgccgaggttgtgagttcgatcctcacctggagca

SEQ ID NO: 129 - DNA, 93 bp, Homo sapiens
gctccagtggcgcaatcggttagcgcgcggtacttatacagcagtacatgcagagcaatgccgaggttgtgagttcgagcctcacctggagca

SEQ ID NO: 130 - DNA, 94 bp, Homo sapiens
gctccagtggcgcaatcggttagcgcgcggtacttatatggcagtatgtgtgcgagtgatgccgaggttgtgagttcgagcctcacctggagca

SEQ ID NO: 131 - DNA, 94 bp, Homo sapiens
gctccagtggcgcaatcggttagcgcgcggtacttatacaacagtatatgtgcgggtgatgccgaggttgtgagttcgagcctcacctggagca

SEQ ID NO: 132 - DNA, 94 bp, Homo sapiens
gctccagtggcgcaatcggttagcgcgcggtacttataagacagtgcacctgtgagcaatgccgaggttgtgagttcaagcctcacctggagca

SEQ ID NO: 133 - DNA, 82 bp, Homo sapiens
ggtagcgtggccgagcggtctaaggcgctggattaaggctccagtctcttcggaggcgtgggttcgaatcccaccgctgcca

SEQ ID NO: 134 - DNA, 82 bp, Homo sapiens
ggtagcgtggccgagcggtctaaggcgctggattaaggctccagtctcttcgggggcgtgggttcgaatcccaccgctgcca

SEQ ID NO: 135 - DNA, 82 bp, Homo sapiens
ggtagcgtggccgagcggtctaaggcgctggattaaggctccagtctcttcgggggcgtgggttcaaatcccaccgctgcca

SEQ ID NO: 136 - DNA, 82 bp, Homo sapiens
ggtagcgtggccgagtggtctaagacgctggattaaggctccagtctcttcgggggcgtgggtttgaatcccaccgctgcca

SEQ ID NO: 137 - DNA, 106 bp, Homo sapiens
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, Homo sapiens
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, Homo sapiens
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, Homo sapiens
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, Homo sapiens
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, Homo sapiens
gcctccttagtgcagtaggtagcgcatcagtctcaaaatctgaatggtcctgagttcaagcctcagagggggca

SEQ ID NO: 143 - DNA, 84 bp, Homo sapiens
gtcaggatggccgagcagtcttaaggcgctgcgttcaaatcgcaccctccgctggaggcgtgggttcgaatcccacttttgaca

SEQ ID NO: 144 - DNA, 83 bp, Homo sapiens
gtcaggatggccgagcggtctaaggcgctgcgttcaggtcgcagtctcccctggaggcgtgggttcgaatcccactcctgaca

SEQ ID NO: 145 - DNA, 83 bp, Homo sapiens
gtcaggatggccgagcggtctaaggcgctgcgttcaggtcgcagtctcccctggaggcgtgggttcgaatcccacttctgaca

SEQ ID NO: 146 - DNA, 83 bp, Homo sapiens
accaggatggccgagtggttaaggcgttggacttaagatccaatggacatatgtccgcgtgggttcgaaccccactcctggta

SEQ ID NO: 147 - DNA, 83 bp, Homo sapiens
accgggatggccgagtggttaaggcgttggacttaagatccaatgggctggtgcccgcgtgggttcgaaccccactctcggta

SEQ ID NO: 148 - DNA, 83 bp, Homo sapiens
accagaatggccgagtggttaaggcgttggacttaagatccaatggattcatatccgcgtgggttcgaaccccacttctggta

SEQ ID NO: 149 - DNA, 83 bp, Homo sapiens
accgggatggctgagtggttaaggcgttggacttaagatccaatggacaggtgtccgcgtgggttcgagccccactcccggta

SEQ ID NO: 150 - DNA, 82 bp, Homo sapiens
ggtagcgtggccgagcggtctaaggcgctggatttaggctccagtctcttcggaggcgtgggttcgaatcccaccgctgcca

SEQ ID NO: 151 - DNA, 82 bp, Homo sapiens
ggtagtgtggccgagcggtctaaggcgctggatttaggctccagtctcttcgggggcgtgggttcgaatcccaccactgcca

SEQ ID NO: 152 - DNA, 82 bp, Homo sapiens
ggtagcgtggccgagtggtctaaggcgctggatttaggctccagtcatttcgatggcgtgggttcgaatcccaccgctgcca

SEQ ID NO: 153 - DNA, 73 bp, Homo sapiens
gcccggctagctcagtcggtagagcatgggactcttaatcccagggtcgtgggttcgagccccacgttgggcg

SEQ ID NO: 154 - DNA, 73 bp, Homo sapiens
gcccagctagctcagtcggtagagcataagactcttaatctcagggttgtggattcgtgccccatgctgggtg

SEQ ID NO: 155 - DNA, 74 bp, Homo sapiens
gcagctagctcagtcggtagagcatgagactcttaatctcagggtcatgggttcgtgccccatgttgggtgcca

SEQ ID NO: 156 - DNA, 73 bp, Homo sapiens
gcccggctagctcagtcggtagagcatgagactcttaatctcagggtcgtgggttcgagccccacgttgggcg

SEQ ID NO: 157 - DNA, 73 bp, Homo sapiens
gcccggctagctcagtcggtagagcatgagacccttaatctcagggtcgtgggttcgagccccacgttgggcg

SEQ ID NO: 158 - DNA, 73 bp, Homo sapiens
gcccggctagctcagtcggtagagcatgggactcttaatctcagggtcgtgggttcgagccccacgttgggcg

SEQ ID NO: 159 - DNA, 73 bp, Homo sapiens
gcccggctagctcagtcgatagagcatgagactcttaatctcagggtcgtgggttcgagccgcacgttgggcg

SEQ ID NO: 160 - DNA, 73 bp, Homo sapiens
gcccagctagctcagtcggtagagcatgagactcttaatctcagggtcatgggtttgagccccacgtttggtg

SEQ ID NO: 161 - DNA, 73 bp, Homo sapiens
gcctggctagctcagtcggcaaagcatgagactcttaatctcagggtcgtgggctcgagctccatgttgggcg

SEQ ID NO: 162 - DNA, 73 bp, Homo sapiens
gcccgactacctcagtcggtggagcatgggactcttcatcccagggttgtgggttcgagccccacattgggca

SEQ ID NO: 163 - DNA, 73 bp, Homo sapiens
gcctggatagctcagttggtagagcatcagacttttaatctgagggtccagggttcaagtccctgttcaggca

SEQ ID NO: 164 - DNA, 73 bp, Homo sapiens
acccagatagctcagtcagtagagcatcagacttttaatctgagggtccaaggttcatgtccctttttgggtg

SEQ ID NO: 165 - DNA, 73 bp, Homo sapiens
gcctggatagctcagttggtagagcatcagacttttaatctgagggtccagggttcaagtccctgttcaggcg

SEQ ID NO: 166 - DNA, 73 bp, Homo sapiens
gcccggatagctcagtcggtagagcatcagacttttaatctgagggtccagggttcaagtccctgttcgggcg

SEQ ID NO: 167 - DNA, 73 bp, Homo sapiens
gcctggatagctcagtcggtagagcatcagacttttaatctgagggtccagggttcaagtccctgttcaggcg

SEQ ID NO: 168 - DNA, 73 bp, Homo sapiens
gcccggatagctcagtcggtagagcatcagacttttaatctgagggtccggggttcaagtccctgttcgggcg

SEQ ID NO: 169 - DNA, 73 bp, Homo sapiens
gcctgggtagctcagtcggtagagcatcagacttttaatctgagggtccagggttcaagtccctgtccaggcg

SEQ ID NO: 170 - DNA, 73 bp, Homo sapiens
gcctggatagctcagttggtagaacatcagacttttaatctgacggtgcagggttcaagtccctgttcaggcg

SEQ ID NO: 171 - DNA, 73 bp, Homo sapiens
gcctcgttagcgcagtaggtagcgcgtcagtctcataatctgaaggtcgtgagttcgatcctcacacggggca

SEQ ID NO: 172 - DNA, 73 bp, Homo sapiens
gccctcttagcgcagtgggcagcgcgtcagtctcataatctgaaggtcctgagttcgagcctcagagagggca

SEQ ID NO: 173 - DNA, 73 bp, Homo sapiens
gcctccttagcgcagtaggcagcgcgtcagtctcataatctgaaggtcctgagttcgaacctcagagggggca

SEQ ID NO: 174 - DNA, 73 bp, Homo sapiens
gccctcttagcgcagcgggcagcgcgtcagtctcataatctgaaggtcctgagttcgagcctcagagagggca

SEQ ID NO: 175 - DNA, 73 bp, Homo sapiens
gccctcttagcgcagctggcagcgcgtcagtctcataatctgaaggtcctgagttcaagcctcagagagggca

SEQ ID NO: 176 - DNA, 73 bp, Homo sapiens
gcctcgttagcgcagtaggcagcgcgtcagtctcataatctgaaggtcgtgagttcgagcctcacacggggca

SEQ ID NO: 177 - DNA, 73 bp, Homo sapiens
gccctcttagtgcagctggcagcgcgtcagtttcataatctgaaagtcctgagttcaagcctcagagagggca

SEQ ID NO: 178 - DNA, 73 bp, Homo sapiens
gccgaaatagctcagttgggagagcgttagactgaagatctaaaggtccctggttcgatcccgggtttcggca

SEQ ID NO: 179 - DNA, 73 bp, Homo sapiens
gccgaaatagctcagttgggagagcgttagactgaagatctaaaggtccctggttcaatcccgggtttcggca

SEQ ID NO: 180 - DNA, 73 bp, Homo sapiens
gccgagatagctcagttgggagagcgttagactgaagatctaaaggtccctggttcaatcccgggtttcggca

SEQ ID NO: 181 - DNA, 74 bp, Homo sapiens
gccgaaatagctcagttgggagagcgttagaccgaagatcttaaaggtccctggttcaatcccgggtttcggca

SEQ ID NO: 182 - DNA, 74 bp, Homo sapiens
gctgaaatagctcagttgggagagcgttagactgaagatcttaaagttccctggttcaaccctgggtttcagcc

SEQ ID NO: 183 - DNA, 72 bp, Homo sapiens
ggctcgttggtctaggggtatgattctcgcttaggatgcgagaggtcccgggttcaaatcccggacgagccc

SEQ ID NO: 184 - DNA, 72 bp, Homo sapiens
ggctcgttggtctaggggtatgattctcgcttagggtgcgagaggtcccgggttcaaatcccggacgagccc

SEQ ID NO: 185 - DNA, 72 bp, Homo sapiens
ggctcgttggtctaggggtatgattctcgcttcgggtgcgagaggtcccgggttcaaatcccggacgagccc

SEQ ID NO: 186 - DNA, 72 bp, Homo sapiens
ggctcgttggtctaggggtatgattctcgcttcgggtgtgagaggtcccgggttcaaatcccggacgagccc

SEQ ID NO: 187 - DNA, 72 bp, Homo sapiens
ggctcgttggtctagtggtatgattctcgctttgggtgcgagaggtcccgggttcaaatcccggacgagccc

SEQ ID NO: 188 - DNA, 72 bp, Homo sapiens
ggctcgttggtctaggggtatgattctcggtttgggtccgagaggtcccgggttcaaatcccggacgagccc

SEQ ID NO: 189 - DNA, 72 bp, Homo sapiens
ggctcgttggtctaggggtatgattctcgctttgggtgcgagaggtcccgggttcaaatcccggacgagccc

SEQ ID NO: 190 - DNA, 87 bp, Homo sapiens
gcccggatgatcctcagtggtctggggtgcaggcttcaaacctgtagctgtctagcgacagagtggttcaattccacctttcgggcg

SEQ ID NO: 191 - DNA, 84 bp, Homo sapiens
gctcggatgatcctcagtggtctggggtgcaggcttcaaacctgtagctgtctagtgacagagtggttcaattccacctttgta

SEQ ID NO: 192 - DNA, 82 bp, Homo sapiens
gtagtcgtggccgagtggttaaggcgatggactagaaatccattggggtttccccgcgcaggttcgaatcctgccgactacg

SEQ ID NO: 193 - DNA, 82 bp, Homo sapiens
gtagtcgtggccgagtggttaaggcgatggactagaaatccattggggtctccccgcgcaggttcgaatcctgccgactacg

SEQ ID NO: 194 - DNA, 82 bp, Homo sapiens
gtagtcgtggccgagtggttaaggcgatggactagaaatccattggggtttccccacgcaggttcgaatcctgccgactacg

SEQ ID NO: 195 - DNA, 82 bp, Homo sapiens
gtagtcgtggccgagtggttaaggtgatggactagaaacccattggggtctccccgcgcaggttcgaatcctgccgactacg

SEQ ID NO: 196 - DNA, 82 bp, Homo sapiens
gctgtgatggccgagtggttaaggcgttggactcgaaatccaatggggtctccccgcgcaggttcgaatcctgctcacagcg

SEQ ID NO: 197 - DNA, 82 bp, Homo sapiens
gctgtgatggccgagtggttaaggcgttggactcgaaatccaatggggtctccccgcgcaggttcaaatcctgctcacagcg

SEQ ID NO: 198 - DNA, 82 bp, Homo sapiens
gctgtgatggccgagtggttaaggtgttggactcgaaatccaatgggggttccccgcgcaggttcaaatcctgctcacagcg

SEQ ID NO: 199 - DNA, 82 bp, Homo sapiens
gtcacggtggccgagtggttaaggcgttggactcgaaatccaatggggtttccccgcacaggttcgaatcctgttcgtgacg

SEQ ID NO: 200 - DNA, 82 bp, Homo sapiens
gacgaggtggccgagtggttaaggcgatggactgctaatccattgtgctctgcacgcgtgggttcgaatcccaccctcgtcg

SEQ ID NO: 201 - DNA, 82 bp, Homo sapiens
gacgaggtggccgagtggttaaggcgatggactgctaatccattgtgctctgcacgcgtgggttcgaatcccaccttcgtcg

SEQ ID NO: 202 - DNA, 82 bp, Homo sapiens
gacgaggtggccgagtggttaaggcgatggactgctaatccattgtgctttgcacgcgtgggttcgaatcccatcctcgtcg

SEQ ID NO: 203 - DNA, 82 bp, Homo sapiens
gacgaggtggccgagtggttaaggcgatggactgctaatccattgtgctctgcacgcgtgggttcgaatcccatcctcgtcg

SEQ ID NO: 204 - DNA, 82 bp, Homo sapiens
gacgaggtggccgagtggttaaggcgatggactgctaatccattgtgctctgcacacgtgggttcgaatcccatcctcgtcg

SEQ ID NO: 205 - DNA, 84 bp, Homo sapiens
ggagaggcctggccgagtggttaaggcgatggactgctaatccattgtgctctgcacgcgtgggttcgaatcccatcctcgtcg

SEQ ID NO: 206 - DNA, 82 bp, Homo sapiens
gcagcgatggccgagtggttaaggcgttggacttgaaatccaatggggtctccccgcgcaggttcgaaccctgctcgctgcg

SEQ ID NO: 207 - DNA, 82 bp, Homo sapiens
gtagtcgtggccgagtggttaaggcgatggacttgaaatccattggggtttccccgcgcaggttcgaatcctgccgactacg

SEQ ID NO: 208 - DNA, 82 bp, Homo sapiens
gtagtcgtggccgagtggttaaggcgatggacttgaaatccattggggtctccccgcgcaggttcgaatcctgccgactacg

SEQ ID NO: 209 - DNA, 82 bp, Homo sapiens
gtagtcgtggccgagtggttaaggcgatggacttgaaatccattggggtttccccgcgcaggttcgaatcctgtcggctacg

SEQ ID NO: 210 - DNA, 74 bp, Homo sapiens
ggcgccgtggcttagttggttaaagcgcctgtctagtaaacaggagatcctgggttcgaatcccagcggtgcct

SEQ ID NO: 211 - DNA, 74 bp, Homo sapiens
ggctccgtggcttagctggttaaagcgcctgtctagtaaacaggagatcctgggttcgaatcccagcggggcct

SEQ ID NO: 212 - DNA, 74 bp, Homo sapiens
ggctccgtagcttagttggttaaagcgcctgtctagtaaacaggagatcctgggttcgactcccagcggggcct

SEQ ID NO: 213 - DNA, 74 bp, Homo sapiens
ggcttcgtggcttagctggttaaagcgcctgtctagtaaacaggagatcctgggttcgaatcccagcgaggcct

SEQ ID NO: 214 - DNA, 74 bp, Homo sapiens
ggcgccgtggcttagctggttaaagcgcctgtctagtaaacaggagatcctgggttcgaatcccagcggtgcct

SEQ ID NO: 215 - DNA, 74 bp, Homo sapiens
ggccctgtggcttagctggtcaaagcgcctgtctagtaaacaggagatcctgggttcgaatcccagcggggcct

SEQ ID NO: 216 - DNA, 74 bp, Homo sapiens
ggctctatggcttagttggttaaagcgcctgtctcgtaaacaggagatcctgggttcgactcccagtggggcct

SEQ ID NO: 217 - DNA, 72 bp, Homo sapiens
ggcgcggtggccaagtggtaaggcgtcggtctcgtaaaccgaagatcacgggttcgaaccccgtccgtgcct

SEQ ID NO: 218 - DNA, 74 bp, Homo sapiens
ggctctgtggcttagttggctaaagcgcctgtctcgtaaacaggagatcctgggttcgaatcccagcggggcct

SEQ ID NO: 219 - DNA, 72 bp, Homo sapiens
ggcgcggtggccaagtggtaaggcgtcggtctcgtaaaccgaagatcgcgggttcgaaccccgtccgtgcct

SEQ ID NO: 220 - DNA, 74 bp, Homo sapiens
ggccctgtagctcagcggttggagcgctggtctcgtaaacctaggggtcgtgagttcaaatctcaccagggcct

SEQ ID NO: 221 - DNA, 74 bp, Homo sapiens
ggctctatggcttagttggttaaagcgcctgtcttgtaaacaggagatcctgggttcgaatcccagtagagcct

SEQ ID NO: 222 - DNA, 73 bp, Homo sapiens
ggctccatagctcagtggttagagcactggtcttgtaaaccaggggtcgcgagttcgatcctcgctggggcct

SEQ ID NO: 223 - DNA, 73 bp, Homo sapiens
ggctccatagctcaggggttagagcgctggtcttgtaaaccaggggtcgcgagttcaattctcgctggggcct

SEQ ID NO: 224 - DNA, 73 bp, Homo sapiens
ggctccatagctcaggggttagagcactggtcttgtaaaccaggggtcgcgagttcaaatctcgctggggcct

SEQ ID NO: 225 - DNA, 73 bp, Homo sapiens
ggccctatagctcaggggttagagcactggtcttgtaaaccaggggtcgcgagttcaaatctcgctggggcct

SEQ ID NO: 226 - DNA, 72 bp, Homo sapiens
ggctccatagctcaggggttagagcactggtcttgtaaaccagggtcgcgagttcaaatctcgctggggcct

SEQ ID NO: 227 - DNA, 72 bp, Homo sapiens
ggcctcgtggcgcaacggtagcgcgtctgactccagatcagaaggttgcgtgttcaaatcacgtcggggtca

SEQ ID NO: 228 - DNA, 72 bp, Homo sapiens
gacctcgtggcgcaatggtagcgcgtctgactccagatcagaaggttgcgtgttcaagtcacgtcggggtca

SEQ ID NO: 229 - DNA, 72 bp, Homo sapiens
gacctcgtggcgcaacggtagcgcgtctgactccagatcagaaggttgcgtgttcaaatcacgtcggggtca

SEQ ID NO: 230 - DNA, 72 bp, Homo sapiens
gacctcgtggcgcaacggtagcgcgtctgactccagatcagaaggctgcgtgttcgaatcacgtcggggtca

SEQ ID NO: 231 - DNA, 72 bp, Homo sapiens
gacctcgtggcgcaacggcagcgcgtctgactccagatcagaaggttgcgtgttcaaatcacgtcggggtca

SEQ ID NO: 232 - DNA, 93 bp, Homo sapiens
ccttcaatagttcagctggtagagcagaggactatagctacttcctcagtaggagacgtccttaggttgctggttcgattccagcttgaagga

SEQ ID NO: 233 - DNA, 91 bp, Homo sapiens
ccttcgatagctcagttggtagagcggaggactgtagttggctgtgtccttagacatccttaggtcgctggttcgaatccggctcgaagga

SEQ ID NO: 234 - DNA, 89 bp, Homo sapiens
ccttcgatagctcagttggtagagcggaggactgtagtggatagggcgtggcaatccttaggtcgctggttcgattccggctcgaagga

SEQ ID NO: 235 - DNA, 89 bp, Homo sapiens
ccttcgatagctcagttggtagagcggaggactgtaggctcattaagcaaggtatccttaggtcgctggttcgaatccggctcggagga

SEQ ID NO: 236 - DNA, 94 bp, Homo sapiens
ccttcgatagctcagctggtagagcggaggactgtagattgtatagacatttgcggacatccttaggtcgctggttcgattccagctcgaagga

SEQ ID NO: 237 - DNA, 93 bp, Homo sapiens
ccttcgatagctcagctggtagagcggaggactgtagctacttcctcagcaggagacatccttaggtcgctggttcgattccggctcgaagga

SEQ ID NO: 238 - DNA, 89 bp, Homo sapiens
ccttcgatagctcagctggtagagcggaggactgtaggcgcgcgcccgtggccatccttaggtcgctggttcgattccggctcgaagga

SEQ ID NO: 239 - DNA, 94 bp, Homo sapiens
ccttcgatagctcagctggtagagcggaggactgtagcctgtagaaacatttgtggacatccttaggtcgctggttcgattccggctcgaagga

SEQ ID NO: 240 - DNA, 94 bp, Homo sapiens
ccttcgatagctcagctggtagagcggaggactgtagattgtacagacatttgcggacatccttaggtcgctggttcgattccggctcgaagga

SEQ ID NO: 241 - DNA, 89 bp, Homo sapiens
ccttcgatagctcagctggtagagcggaggactgtagtacttaatgtgtggtcatccttaggtcgctggttcgattccggctcgaagga

SEQ ID NO: 242 - DNA, 89 bp, Homo sapiens
ccttcgatagctcagctggtagagcggaggactgtaggggtttgaatgtggtcatccttaggtcgctggttcgaatccggctcggagga

SEQ ID NO: 243 - DNA, 94 bp, Homo sapiens
ccttcgatagctcagctggtagagcggaggactgtagactgcggaaacgtttgtggacatccttaggtcgctggttcaattccggctcgaagga

SEQ ID NO: 244 - DNA, 90 bp, Homo sapiens
ctttcgatagctcagttggtagagcggaggactgtaggttcattaaactaaggcatccttaggtcgctggttcgaatccggctcgaagga

SEQ ID NO: 245 - DNA, 88 bp, Homo sapiens
tcttcaatagctcagctggtagagcggaggactgtaggtgcacgcccgtggccattcttaggtgctggtttgattccgacttggagag

SEQ ID NO: 246 - DNA, 73 bp, Homo sapiens
gtttccgtagtgtagtggttatcacgttcgcctaacacgcgaaaggtccccggttcgaaaccgggcggaaaca

SEQ ID NO: 247 - DNA, 73 bp, Homo sapiens
gtttccgtagtgtagtggtcatcacgttcgcctaacacgcgaaaggtccccggttcgaaaccgggcggaaaca

SEQ ID NO: 248 - DNA, 73 bp, Homo sapiens
gtttccgtagtgtagtggttatcacgttcgcctaacacgcgaaaggtccctggatcaaaaccaggcggaaaca

SEQ ID NO: 249 - DNA, 73 bp, Homo sapiens
gtttccgtagtgtagtggttatcacgttcgcctaacacgcgaaaggtccgcggttcgaaaccgggcggaaaca

SEQ ID NO: 250 - DNA, 73 bp, Homo sapiens
gtttccgtagtgtagtggttatcacgtttgcctaacacgcgaaaggtccccggttcgaaaccgggcagaaaca

SEQ ID NO: 251 - DNA, 72 bp, Homo sapiens
gggggtgtagctcagtggtagagcgtatgcttaacattcatgaggctctgggttcgatccccagcacttcca

SEQ ID NO: 252 - DNA, 73 bp, Homo sapiens
gtttccgtagtgtagtggttatcacgttcgcctcacacgcgaaaggtccccggttcgaaaccgggcggaaaca

SEQ ID NO: 253 - DNA, 73 bp, Homo sapiens
gcttctgtagtgtagtggttatcacgttcgcctcacacgcgaaaggtccccggttcgaaaccgggcagaagca

SEQ ID NO: 254 - DNA, 73 bp, Homo sapiens
gtttccgtagtgtagcggttatcacattcgcctcacacgcgaaaggtccccggttcgatcccgggcggaaaca

SEQ ID NO: 255 - DNA, 73 bp, Homo sapiens
gtttccgtagtgtagtggttatcacgttcgcctcacacgcgaaaggtccccggttcgaaactgggcggaaaca

SEQ ID NO: 256 - DNA, 74 bp, Homo sapiens
gtttccgtagtgtagtggttatcacgttcgcctcacacgcgtaaaggtccccggttcgaaaccgggcggaaaca

SEQ ID NO: 257 - DNA, 73 bp, Homo sapiens
gtttccgtagtggagtggttatcacgttcgcctcacacgcgaaaggtccccggtttgaaaccaggcggaaaca

SEQ ID NO: 258 - DNA, 73 bp, Homo sapiens
ggttccatagtgtagtggttatcacgtctgctttacacgcagaaggtcctgggttcgagccccagtggaacca

SEQ ID NO: 259 - DNA, 73 bp, Homo sapiens
ggttccatagtgtagcggttatcacgtctgctttacacgcagaaggtcctgggttcgagccccagtggaacca

SEQ ID NO: 260 - DNA, 73 bp, Homo sapiens
ggttccatagtgtagtggttatcacatctgctttacacgcagaaggtcctgggttcaagccccagtggaacca

SEQ ID NO: 261 - DNA, 73 bp, Homo sapiens
gtttccgtggtgtagtggttatcacattcgccttacacgcgaaaggtcctcgggtcgaaaccgagcggaaaca

SEQ ID NO: 262 - DNA, 72 bp, Homo sapiens
agcagagtggcgcagcggaagcgtgctgggcccataacccagaggtcgatggatcgaaaccatcctctgcta

SEQ ID NO: 263 - DNA, 72 bp, Homo sapiens
agcagagtggcgcagcggaagcgtgctgggcccataacccagaggtcgatggatctaaaccatcctctgcta

SEQ ID NO: 264 - DNA, 73 bp, Homo sapiens
tccctggtggtctagtggctaggattcggcgctttcaccgccgcggcccgggttcgattcccggtcagggaat

SEQ ID NO: 265 - DNA, 72 bp, Homo sapiens
gcgttggtggtttagtggtagaattctcgcctcccatgcgggagacccgggttcaattcccggccactgcac

SEQ ID NO: 266 - DNA, 72 bp, Homo sapiens
ggccttggtggtgcagtggtagaattctcgcctcccacgtgggagacccgggttcaattcccggccaatgca

SEQ ID NO: 267 - DNA, 73 bp, Homo sapiens
gtccctggtggtctagtggctaggattcggcgctttcaccgccgcggcccgggttcgattcccggccagggaa

SEQ ID NO: 268 - DNA, 75 bp, Homo sapiens
tgtctctgtggcgcaatcggttagcgcgttcggctgttaaccgaaagattggtggttcgagcccacccagggacg

SEQ ID NO: 269 - DNA, 86 bp, Homo sapiens
tggctccgtggcgcaatggatagcgcattggacttctagaggctgaaggcattcaaaggttccgggttcgagtcccggcggagtcg

SEQ ID NO: 270 - DNA, 74 bp, Homo sapiens
gcccggctagctcagtcggtagagcatgagactcttaatctcagggtcgtgggttcgagccccacgttgggcgc

SEQ ID NO: 271 - DNA, 73 bp, Homo sapiens
gccgtgatcgtatagtggttagtactctgcgttgtggccgcagcaacctcggttcgaatccgagtcacggcag

SEQ ID NO: 272 - DNA, 73 bp, Homo sapiens
gcgttggtggtatagtggtgagcatagctgccttccaagcagttgacccgggttcgattcccggccaacgcag

SEQ ID NO: 273 - DNA, 73 bp, Homo sapiens
tccctggtggtctagtggttaggattcggcgctctcaccgccgcggcccgggttcgattcccggtcagggaaa

SEQ ID NO: 274 - DNA, 73 bp, Homo sapiens
aggttccatggtgtaatggtgagcactctggactctgaatccagcgatccgagttcgagtctcggtggaacct

SEQ ID NO: 275 - DNA, 75 bp, Homo sapiens
tgtctctgtggcgtagtcggttagcgcgttcggctgttaaccgaaaagttggtggttcgagcccacccaggaacg

SEQ ID NO: 276 - DNA, 75 bp, Homo sapiens
tgtctctgtggcgcaatcggttagcgcgttcggctgttaaccgaaaggttggtggttcgagcccacccagggacg

SEQ ID NO: 277 - DNA, 75 bp, Homo sapiens
gtctctgtggcgcaatcggttagcgcattcggctgttaaccgaaaggttggtggttcgagcccacccagggacgc

SEQ ID NO: 278 - DNA, 75 bp, Homo sapiens
gtctctgtggcgcaatgggttagcgcgttcggctgttaaccgaaaggttggtggttcgagcccatccagggacgc

SEQ ID NO: 279 - DNA, 72 bp, Homo sapiens
gcactggtggttcagtggtagaattctcgcctcacacgcgggacacccgggttcaattcccggtcaaggcaa

SEQ ID NO: 280 - DNA, 74 bp, Homo sapiens
gtttccgtagtgtagtggttatcacgttcgcctcacacgcgaaaggtccccggttcgaaactgggcggaaacag

SEQ ID NO: 281 - DNA, 72 bp, Homo sapiens
gcactggtggttcagtggtagaattctcgcctcccacgcgggagacccgggtttaattcccggtcaagataa

SEQ ID NO: 282 - DNA, 75 bp, Homo sapiens
gtttccgtagtgtagtggttatcacgttcgcctcacacgcgtaaaggtccccggttcgaaaccgggcggaaacat

SEQ ID NO: 283 - DNA, 73 bp, Homo sapiens
tagcagagtggcgcagcggaagcgtgctgggcccataacccagaggtcgatggatcgaaaccatcctctgcta

SEQ ID NO: 284 - DNA, 74 bp, Homo sapiens
gtttccgtagtgtagtggttatcacgttcgcctcacacgcgaaaggtccccggttcgaaaccgggcggaaacaa

SEQ ID NO: 285 - DNA, 73 bp, Homo sapiens
tcctcgttagtatagtggtgagtatccccgcctgtcacgcgggagaccggggttcgattccccgacggggagg

SEQ ID NO: 286 - DNA, 72 bp, Homo sapiens
tgcatgggtggttcagtggtagaattctcgcctgccacgcgggaggcccgggttcgattcccggcccatgca

SEQ ID NO: 287 - DNA, 73 bp, Homo sapiens
tccctggtggtctagtggttaggattcggcgctctcaccgccgcggcccgggttcgattcccggtcagggaag

SEQ ID NO: 288 - DNA, 73 bp, Homo sapiens
atccttgttactatagtggtgagtatctctgcctgtcatgcgtgagagagggggtcgattccccgacggggag

SEQ ID NO: 289 - DNA, 72 bp, Homo sapiens
gcattggtggttcagtggtagaattctcgcctgccacgcgggaggcccgggttcgattcccggccaatgcac

SEQ ID NO: 290 - DNA, 84 bp, Homo sapiens
gtcaggatggccgagcggtctaaggcgctgcgttcaggtcgcagtctcccctggaggcgtgggttcgaatcccactcctgacaa

SEQ ID NO: 291 - DNA, 73 bp, Homo sapiens
cgcgttggtggtatagtggtgagcatagctgccttccaagcagttgacccgggttcgattcccggccaacgca

SEQ ID NO: 292 - DNA, 75 bp, Homo sapiens
cgtctctgtggcgcaatcggttagcgcgttcggctgttaaccgaaaggttggtggttcgatcccacccagggacg

SEQ ID NO: 293 - DNA, 73 bp, Homo sapiens
cgcgttggtggtgtagtggtgagcacagctgcctttcaagcagttaacgcgggttcgattcccgggtaacgaa

SEQ ID NO: 294 - DNA, 73 bp, Homo sapiens
cggctcgttggtctaggggtatgattctcgcttcgggtgcgagaggtcccgggttcaaatcccggacgagccc

SEQ ID NO: 295 - DNA, 73 bp, Homo sapiens
ggctcgttggtctaggggtatgattctcgcttagggtgcgagaggtcccgggttcaaatcccggacgagccct

SEQ ID NO: 296 - DNA, 74 bp, Homo sapiens
cgcccggatagctcagtcggtagagcatcagacttttaatctgagggtccagggttcaagtccctgttcgggcg

SEQ ID NO: 297 - DNA, 74 bp, Homo sapiens
gcccggatagctcagtcggtagagcatcagacttttaatctgagggtccagggttcaagtccctgttcgggcgt

SEQ ID NO: 298 - DNA, 107 bp, Homo sapiens
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, Homo sapiens
ttccctggtggtctagtggttaggattcggcgctctcaccgccgcggcccgggttcgattcccggtcaggaaa

SEQ ID NO: 300 - DNA, 90 bp, Homo sapiens
gccttcgatagctcagttggtagagcggaggactgtagtggatagggcgtggcaatccttaggtcgctggttcgattccggctcgaagga

SEQ ID NO: 301 - DNA, 74 bp, Homo sapiens
cgggggattagctcaaatggtagagcgctcgcttagcatgcgagaggtagcgggatcgatgcccgcatcctcca

SEQ ID NO: 302 - DNA, 94 bp, Homo sapiens
agctccagtggcgcaatcggttagcgcgcggtacttatacagcagtacatgcagagcaatgccgaggttgtgagttcgagcctcacctggagca

SEQ ID NO: 303 - DNA, 72 bp, Homo sapiens
gcgccgctggtgtagtggtatcatgcaagattcccattcttgcgacccgggttcgattcccgggcggcgcat

SEQ ID NO: 304 - DNA, 73 bp, Homo sapiens
tcccatatggtctagcggttaggattcctggttttcacccaggtggcccgggttcgactcccggtatgggaac

SEQ ID NO: 305 - DNA, 73 bp, Homo sapiens
gggggatgtagctcagtggtagagcgcgcgcttcgcatgtgtgaggtcccgggttcaatccccggcatctcca

SEQ ID NO: 306 - DNA, 72 bp, Homo sapiens
gcattggtggttcagtggtagaattctcgcctgccacgcgggaggcccgggttcgattcccggccaatgcaa

SEQ ID NO: 307 - DNA, 74 bp, Homo sapiens
gggccagtggcgcaatggataacgcgtctgactacggatcagaagattctaggttcgactcctggctggctcgc

SEQ ID NO: 308 - DNA, 74 bp, Homo sapiens
ggtttccgtagtgtagtggttatcacgttcgcctaacacgcgaaaggtccccggttcgaaaccgggcggaaaca

SEQ ID NO: 309 - DNA, 74 bp, Homo sapiens
agtttccgtagtgtagtggttatcacgttcgcctaacacgcgaaaggtccccggttcgaaaccgggcggaaaca

SEQ ID NO: 310 - DNA, 83 bp, Homo sapiens
aggtagcgtggccgagcggtctaaggcgctggattaaggctccagtctcttcgggggcgtgggttcgaatcccaccgctgcca

SEQ ID NO: 311 - DNA, 74 bp, Homo sapiens
gtttccgtagtgtagtggtcatcacgttcgcctaacacgcgaaaggtccccggttcgaaaccgggcggaaacat

SEQ ID NO: 312 - DNA, 73 bp, Homo sapiens
ggctcgttggtctaggggtatgattctcgctttgggtgcgagaggtcccgggttcaaatcccggacgagccca

SEQ ID NO: 313 - DNA, 73 bp, Homo sapiens
ggctccatagctcaggggttagagcactggtcttgtaaaccagggtcgcgagttcaaatctcgctggggcctg

SEQ ID NO: 314 - DNA, 73 bp, Homo sapiens
tggggatgtagctcagtggtagagcgcatgctttgcatgtatgaggccccgggttcgatccccggcatctcca

SEQ ID NO: 315 - DNA, 74 bp, Homo sapiens
cgcccggctagctcagtcggtagagcatgagactcttaatctcagggtcgtgggttcgagccccacgttgggcg

SEQ ID NO: 316 - DNA, 74 bp, Homo sapiens
gtttccgtagtgtagtggttatcacgttcgcctaacacgcgaaaggtccccggttcgaaaccgggcggaaacaa

SEQ ID NO: 317 - DNA, 74 bp, Homo sapiens
gcccggctagctcagtcggtagagcatgagactcttaatctcagggtcgtgggttcgagccccacgttgggcgt

SEQ ID NO: 318 - DNA, 74 bp, Homo sapiens
gtttccgtagtgtagtggttatcacgttcgcctcacacgcgaaaggtccccggttcgaaaccgggcggaaacac

SEQ ID NO: 319 - DNA, 73 bp, Homo sapiens
cagcagagtggcgcagcggaagcgtgctgggcccataacccagaggtcgatggatcgaaaccatcctctgcta

SEQ ID NO: 320 - DNA, 85 bp, Homo sapiens
ggagaggcctggccgagtggttaaggcgatggactgctaatccattgtgctctgcacgcgtgggttcgaatcccatcctcgtcgc

SEQ ID NO: 321 - DNA, 73 bp, Homo sapiens
ggccccatggtgtaatggttagcactctggactttgaatccagcgatccgagttcaaatctcggtgggacctg

SEQ ID NO: 322 - DNA, 73 bp, Homo sapiens
ggccccatggtgtaatggttagcactctggactttgaatccagcgatccgagttcaaatctcggtgggaccta

SEQ ID NO: 323 - DNA, 83 bp, Homo sapiens
gtagtcgtggccgagtggttaaggcgatggacttgaaatccattggggtctccccgcgcaggttcgaatcctgccgactacgg

SEQ ID NO: 324 - DNA, 73 bp, Homo sapiens
agcagagtggcgcagcggaagcgtgctgggcccataacccagaggtcgatggatcgaaaccatcctctgctat

SEQ ID NO: 325 - DNA, 74 bp, Homo sapiens
ggaccacgtggcctaatggataaggcgtctgacttcggatcagaagattgagggttcgaatccctccgtggtta

SEQ ID NO: 326 - DNA, 83 bp, Homo sapiens
tgtagtcgtggccgagtggttaaggcgatggactagaaatccattggggtctccccgcgcaggttcgaatcctgccgactacg

SEQ ID NO: 327 - DNA, 73 bp, Homo sapiens
agcagagtggcgcagcggaagcgtgctgggcccataacccagaggtcgatggatcgaaaccatcctctgctag

SEQ ID NO: 328 - DNA, 84 bp, Homo sapiens
cgtcaggatggccgagcggtctaaggcgctgcgttcaggtcgcagtctcccctggaggcgtgggttcgaatcccactcctgaca

SEQ ID NO: 329 - DNA, 75 bp, Homo sapiens
ggctccgtggcttagctggttaaagcgcctgtctagtaaacaggagatcctgggttcgaatcccagcggggcctg

SEQ ID NO: 330 - DNA, 74 bp, Homo sapiens
agggccagtggcgcaatggataacgcgtctgactacggatcagaagattccaggttcgactcctggctggctcg

SEQ ID NO: 331 - DNA, 74 bp, Homo sapiens
ggtttccgtagtgtagtggttatcacgttcgcctcacacgcgaaaggtccccggttcgaaaccgggcggaaaca

SEQ ID NO: 332 - DNA, 73 bp, Homo sapiens
aggggatgtagctcagtggtagagcgcatgcttcgcatgtatgaggtcccgggttcgatccccggcatctcca

SEQ ID NO: 333 - DNA, 75 bp, Homo sapiens
tggccggttagctcagttggttagagcgtggtgctaataacgccaaggtcgcgggttcgatccccgtacgggcca

SEQ ID NO: 334 - DNA, 73 bp, Homo sapiens
cggctcgttggtctaggggtatgattctcgcttagggtgcgagaggtcccgggttcaaatcccggacgagccc

SEQ ID NO: 335 - DNA, 74 bp, Homo sapiens
agcccggctagctcagtcggtagagcatgagactcttaatctcagggtcgtgggttcgagccccacgttgggcg

SEQ ID NO: 336 - DNA, 92 bp, Homo sapiens
tccttcgatagctcagttggtagagcggaggactgtagttggctgtgtccttagacatccttaggtcgctggttcgaatccggctcgaagga

SEQ ID NO: 337 - DNA, 74 bp, Homo sapiens
ggggaattagctcaaatggtagagcgctcgcttagcatgcgagaggtagcgggatcgatgcccgcattctccag

SEQ ID NO: 338 - DNA, 74 bp, Homo sapiens
cgccctcttagcgcagcgggcagcgcgtcagtctcataatctgaaggtcctgagttcgagcctcagagagggca

SEQ ID NO: 339 - DNA, 95 bp, Homo sapiens
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, Homo sapiens
tgccgtgatcgtatagtggttagtactctgcgttgtggccgcagcaacctcggttcgaatccgagtcacggca

SEQ ID NO: 341 - DNA, 75 bp, Homo sapiens
ggccggttagctcagttggttagagcgtggtgctaataacgccaaggtcgcgggttcgatccccgtacgggccac

SEQ ID NO: 342 - DNA, 74 bp, Homo sapiens
agtttccgtagtgtagtggttatcacgtttgcctaacacgcgaaaggtccccggttcgaaaccgggcagaaaca

SEQ ID NO: 343 - DNA, 74 bp, Homo sapiens
gcttctgtagtgtagtggttatcacgttcgcctcacacgcgaaaggtccccggttcgaaaccgggcagaagcaa

SEQ ID NO: 344 - DNA, 73 bp, Homo sapiens
ttcctcgttagtatagtggtgagtatccccgcctgtcacgcgggagaccggggttcgattccccgacggggag

SEQ ID NO: 345 - DNA, 83 bp, Homo sapiens
gtagtcgtggccgagtggttaaggcgatggacttgaaatccattggggtttccccgcgcaggttcgaatcctgtcggctacgg

SEQ ID NO: 346 - DNA, 73 bp, Homo sapiens
aggttccatggtgtaatggttagcactctggactctgaatccagcgatccgagttcaaatctcggtggaacct

SEQ ID NO: 347 - DNA, 73 bp, Homo sapiens
tcctcgttagtatagtggtgagtgtccccgtctgtcacgcgggagaccggggttcgattccccgacggggaga

SEQ ID NO: 348 - DNA, 74 bp, Homo sapiens
gtttccgtagtgtagtggttatcacgttcgcctaacacgcgaaaggtccctggatcaaaaccaggcggaaacaa

SEQ ID NO: 349 - DNA, 75 bp, Homo sapiens
cggccggttagctcagttggttagagcgtggtgctaataacgccaaggtcgcgggttcgatccccgtactggcca

SEQ ID NO: 350 - DNA, 73 bp, Homo sapiens
ggccccatggtgtaatggtcagcactctggactctgaatccagcgatccgagttcaaatctcggtgggaccca

SEQ ID NO: 351 - DNA, 73 bp, Homo sapiens
ggccccatggtgtaatggttagcactctggactttgaatccagcgatccgagttcaaatctcggtgggacctt

SEQ ID NO: 352 - DNA, 73 bp, Homo sapiens
tgggggtgtagctcagtggtagagcgcgtgcttagcatgtacgaggtcccgggttcaatccccggcacctcca

SEQ ID NO: 353 - DNA, 73 bp, Homo sapiens
ggggatgtagctcagtggtagagcgcatgcttagcatgcatgaggtcccgggttcgatccccagcatctccag

SEQ ID NO: 354 - DNA, 73 bp, Homo sapiens
agggggtgtagctcagtggtagagcgcgtgcttcgcatgtacgaggccccgggttcgacccccggctcctcca

SEQ ID NO: 355 - DNA, 73 bp, Homo sapiens
gggggtgtagctcagtggtagagcgcgtgcttagcatgcacgaggccccgggttcaatccccggcacctccat

SEQ ID NO: 356 - DNA, 73 bp, Homo sapiens
gggggtgtagctcagtggtagagcgcgtgcttagcatgcacgaggccccgggttcaatccccggcacctccag

SEQ ID NO: 357 - DNA, 107 bp, Homo sapiens
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, Homo sapiens
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, Homo sapiens
ggttccatggtgtaatggttagcactctggactctgaatccagcgatccgagttcaaatctcggtggaacctt

SEQ ID NO: 360 - DNA, 83 bp, Homo sapiens
ggtagcgtggccgagcggtctaaggcgctggattaaggctccagtctcttcgggggcgtgggttcgaatcccaccgctgccag

SEQ ID NO: 361 - DNA, 74 bp, Homo sapiens
tgcctccttagcgcagtaggcagcgcgtcagtctcataatctgaaggtcctgagttcgaacctcagagggggca

SEQ ID NO: 362 - DNA, 74 bp, Homo sapiens
agcccggatagctcagtcggtagagcatcagacttttaatctgagggtccagggttcaagtccctgttcgggcg

SEQ ID NO: 363 - DNA, 74 bp, Homo sapiens
gcctccttagcgcagtaggcagcgcgtcagtctcataatctgaaggtcctgagttcgaacctcagagggggcag

SEQ ID NO: 364 - DNA, 73 bp, Homo sapiens
ttccctggtggtctagtggttaggattcggcgctctcaccgccgcggcccgggttcgattcccggtcagggaa

SEQ ID NO: 365 - DNA, 84 bp, Homo sapiens
caccaggatggccgagtggttaaggcgttggacttaagatccaatggacatatgtccgcgtgggttcgaaccccactcctggta

SEQ ID NO: 366 - DNA, 73 bp, Homo sapiens
tggctcgttggtctaggggtatgattctcgcttagggtgcgagaggtcccgggttcaaatcccggacgagccc

SEQ ID NO: 367 - DNA, 74 bp, Homo sapiens
agccccagtggcctaatggataaggcattggcctcctaagccagggattgtgggttcgagtcccatctggggtg

SEQ ID NO: 368 - DNA, 73 bp, Homo sapiens
ggggatatagctcaggggtagagcatttgactgcagatcaagaggtccccggttcaaatccgggtgccccccc

SEQ ID NO: 369 - DNA, 94 bp, Homo sapiens
cccttcgatagctcagctggtagagcggaggactgtagctacttcctcagcaggagacatccttaggtcgctggttcgattccggctcgaagga

SEQ ID NO: 370 - DNA, 90 bp, Homo sapiens
cccttcgatagctcagctggtagagcggaggactgtaggcgcgcgcccgtggccatccttaggtcgctggttcgattccggctcgaagga

SEQ ID NO: 371 - DNA, 74 bp, Homo sapiens
tgggggattagctcaaatggtagagcgctcgcttagcatgcgagaggtagcgggatcgatgcccgcatcctcca

SEQ ID NO: 372 - DNA, 83 bp, Homo sapiens
gtagtcgtggccgagtggttaaggcgatggactagaaatccattggggtctccccgcgcaggttcgaatcctgccgactacgg

SEQ ID NO: 373 - DNA, 74 bp, Homo sapiens
gcctcgttagcgcagtaggtagcgcgtcagtctcataatctgaaggtcgtgagttcgatcctcacacggggcac

SEQ ID NO: 374 - DNA, 92 bp, Homo sapiens
ggctctgtggcgcaatggatagcgcattggacttctagctgagcctagtgtggtcattcaaaggttgtgggttcgagtcccaccagagtcga

SEQ ID NO: 375 - DNA, 75 bp, Homo sapiens
gtctctgtggcgcaatcggttagcgcgttcggctgttaaccgaaaggttggtggttcgagcccacccagggacgc

SEQ ID NO: 376 - DNA, 83 bp, Homo sapiens
ggcagcgatggccgagtggttaaggcgttggacttgaaatccaatggggtctccccgcgcaggttcgaaccctgctcgctgcg

SEQ ID NO: 377 - DNA, 74 bp, Homo sapiens
ggttccatagtgtagtggttatcacgtctgctttacacgcagaaggtcctgggttcgagccccagtggaaccat

SEQ ID NO: 378 - DNA, 74 bp, Homo sapiens
ggttccatagtgtagcggttatcacgtctgctttacacgcagaaggtcctgggttcgagccccagtggaaccac

SEQ ID NO: 379 - DNA, 87 bp, Homo sapiens
tggctctgtggcgcaatggatagcgcattggacttctagatagttagagaaattcaaaggttgtgggttcgagtcccaccagagtcg

SEQ ID NO: 380 - DNA, 84 bp, Homo sapiens
taccagaatggccgagtggttaaggcgttggacttaagatccaatggattcatatccgcgtgggttcgaaccccacttctggta

SEQ ID NO: 381 - DNA, 74 bp, Homo sapiens
ggcccggatagctcagtcggtagagcatcagacttttaatctgagggtccggggttcaagtccctgttcgggcg

SEQ ID NO: 382 - DNA, 74 bp, Homo sapiens
gccgaaatagctcagttgggagagcgttagactgaagatctaaaggtccctggttcgatcccgggtttcggcag

SEQ ID NO: 383 - DNA, 74 bp, Homo sapiens
gcccggatagctcagtcggtagagcatcagacttttaatctgagggtccagggttcaagtccctgttcgggcgg

SEQ ID NO: 384 - DNA, 74 bp, Homo sapiens
gccgaaatagctcagttgggagagcgttagactgaagatctaaaggtccctggttcaatcccgggtttcggcag

SEQ ID NO: 385 - DNA, 83 bp, Homo sapiens
ggacgaggtggccgagtggttaaggcgatggactgctaatccattgtgctttgcacgcgtgggttcgaatcccatcctcgtcg

SEQ ID NO: 386 - DNA, 73 bp, Homo sapiens
ggctcgttggtctaggggtatgattctcggtttgggtccgagaggtcccgggttcaaatcccggacgagcccc

SEQ ID NO: 387 - DNA, 83 bp, Homo sapiens
agtcacggtggccgagtggttaaggcgttggactcgaaatccaatggggtttccccgcacaggttcgaatcctgttcgtgacg

SEQ ID NO: 388 - DNA, 73 bp, Homo sapiens
ctcctcgttagtatagtggttagtatccccgcctgtcacgcgggagaccggggttcaattccccgacggggag

SEQ ID NO: 389 - DNA, 73 bp, Homo sapiens
ggacctcgtggcgcaacggtagcgcgtctgactccagatcagaaggctgcgtgttcgaatcacgtcggggtca

SEQ ID NO: 390 - DNA, 73 bp, Homo sapiens
ggggatgtagctcagtggtagagcgcatgctttgcatgtatgaggccccgggttcgatccccggcatctccat

SEQ ID NO: 391 - DNA, 74 bp, Homo sapiens
gccgaaatagctcagttgggagagcgttagactgaagatctaaaggtccctggttcgatcccgggtttcggcac

SEQ ID NO: 392 - DNA, 73 bp, Homo sapiens
aggggatgtagctcagtggtagagcgcatgctttgcacgtatgaggccccgggttcaatccccggcatctcca

SEQ ID NO: 393 - DNA, 75 bp, Homo sapiens
gtctctgtggcgcaatcggttagcgcgttcggctgttaaccgaaaggttggtggttcgagcccacccagggacgg

SEQ ID NO: 394 - DNA, 73 bp, Homo sapiens
tcccacatggtctagcggttaggattcctggttttcacccaggcggcccgggttcgactcccggtgtgggaac

SEQ ID NO: 395 - DNA, 74 bp, Homo sapiens
ggctccatagctcaggggttagagcgctggtcttgtaaaccaggggtcgcgagttcaattctcgctggggcctg

SEQ ID NO: 396 - DNA, 83 bp, Homo sapiens
tggtagtgtggccgagcggtctaaggcgctggatttaggctccagtctcttcgggggcgtgggttcgaatcccaccactgcca

SEQ ID NO: 397 - DNA, 74 bp, Homo sapiens
ggctccatagctcaggggttagagcactggtcttgtaaaccaggggtcgcgagttcaaatctcgctggggcctc

SEQ ID NO: 398 - DNA, 73 bp, Homo sapiens
tggctcgttggtctagtggtatgattctcgctttgggtgcgagaggtcccgggttcaaatcccggacgagccc

SEQ ID NO: 399 - DNA, 95 bp, Homo sapiens
ccttcgatagctcagctggtagagcggaggactgtagattgtacagacatttgcggacatccttaggtcgctggttcgattccggctcgaaggaa

SEQ ID NO: 400 - DNA, 74 bp, Homo sapiens
aggccctatagctcaggggttagagcactggtcttgtaaaccaggggtcgcgagttcaaatctcgctggggcct

SEQ ID NO: 401 - DNA, 90 bp, Homo sapiens
tccttcgatagctcagctggtagagcggaggactgtagtacttaatgtgtggtcatccttaggtcgctggttcgattccggctcgaagga

SEQ ID NO: 402 - DNA, 73 bp, Homo sapiens
tggctcgttggtctaggggtatgattctcgctttgggtgcgagaggtcccgggttcaaatcccggacgagccc

SEQ ID NO: 403 - DNA, 74 bp, Homo sapiens
gcccggctagctcagtcggtagagcatgggactcttaatcccagggtcgtgggttcgagccccacgttgggcgc

SEQ ID NO: 404 - DNA, 75 bp, Homo sapiens
cggccggttagctcagttggttagagcgtggtgctaataacgccaaggtcgcgggttcgatccccgtacgggcca

SEQ ID NO: 405 - DNA, 73 bp, Homo sapiens
tcccacatggtctagcggttaggattcctggttttcacccaggcggcccgggttcgactcccggtgtgggaat

SEQ ID NO: 406 - DNA, 83 bp, Homo sapiens
gacgaggtggccgagtggttaaggcgatggactgctaatccattgtgctctgcacgcgtgggttcgaatcccatcctcgtcga

SEQ ID NO: 407 - DNA, 73 bp, Homo sapiens
gccgtgatcgtatagtggttagtactctgcgttgtggccgcagcaacctcggttcgaatccgagtcacggcat

SEQ ID NO: 408 - DNA, 73 bp, Homo sapiens
cgccgtgatcgtatagtggttagtactctgcgttgtggccgcagcaacctcggttcgaatccgagtcacggca

SEQ ID NO: 409 - DNA, 73 bp, Homo sapiens
ggttccatggtgtaatggttagcactctggactctgaatccagcgatccgagttcaaatctcggtggaacctg

SEQ ID NO: 410 - DNA, 74 bp, Homo sapiens
tgcccggctagctcagtcggtagagcatgggactcttaatcccagggtcgtgggttcgagccccacgttgggcg

SEQ ID NO: 411 - DNA, 74 bp, Homo sapiens
gggccgcgtggcctaatggataaggcgtctgacttcggatcagaagattgcaggttcgagtcctgccgcggtcg

SEQ ID NO: 412 - DNA, 72 bp, Homo sapiens
gcgccgctggtgtagtggtatcatgcaagattcccattcttgcgacccgggttcgattcccgggcggcgcac

SEQ ID NO: 413 - DNA, 74 bp, Homo sapiens
gggccgcgtggcctaatggataaggcgtctgattccggatcagaagattgagggttcgagtcccttcgtggtcg

SEQ ID NO: 414 - DNA, 74 bp, Homo sapiens
cgccccggtggcctaatggataaggcattggcctcctaagccagggattgtgggttcgagtcccacccggggta

SEQ ID NO: 415 - DNA, 74 bp, Homo sapiens
gcccggctagctcagtcggtagagcatgagacccttaatctcagggtcgtgggttcgagccccacgttgggcgt

SEQ ID NO: 416 - DNA, 73 bp, Homo sapiens
aggcgcggtggccaagtggtaaggcgtcggtctcgtaaaccgaagatcacgggttcgaaccccgtccgtgcct

SEQ ID NO: 417 - DNA, 83 bp, Homo sapiens
ggtagcgtggccgagtggtctaaggcgctggatttaggctccagtcatttcgatggcgtgggttcgaatcccaccgctgccac

SEQ ID NO: 418 - DNA, 83 bp, Homo sapiens
gggtagcgtggccgagcggtctaaggcgctggattaaggctccagtctcttcgggggcgtgggttcgaatcccaccgctgcca

SEQ ID NO: 419 - DNA, 84 bp, Homo sapiens
agtcaggatggccgagcggtctaaggcgctgcgttcaggtcgcagtctcccctggaggcgtgggttcgaatcccacttctgaca

SEQ ID NO: 420 - DNA, 84 bp, Homo sapiens
gtcaggatggccgagcggtctaaggcgctgcgttcaggtcgcagtctcccctggaggcgtgggttcgaatcccacttctgacag

SEQ ID NO: 421 - DNA, 74 bp, Homo sapiens
gcctcgttagcgcagtaggcagcgcgtcagtctcataatctgaaggtcgtgagttcgagcctcacacggggcag

SEQ ID NO: 422 - DNA, 83 bp, Homo sapiens
ggtagcgtggccgagcggtctaaggcgctggatttaggctccagtctcttcggaggcgtgggttcgaatcccaccgctgccag

SEQ ID NO: 423 - DNA, 89 bp, Homo sapiens
tggctctgtggcgcaatggatagcgcattggacttctagtgacgaatagagcaattcaaaggttgtgggttcgaatcccaccagagtcg

SEQ ID NO: 424 - DNA, 72 bp, Homo sapiens
cgcattggtggttcagtggtagaattctcgcctgccacgcgggaggcccgggttcgattcccggccaatgca

SEQ ID NO: 425 - DNA, 83 bp, Homo sapiens
gctgtgatggccgagtggttaaggcgttggactcgaaatccaatggggtctccccgcgcaggttcgaatcctgctcacagcgt

SEQ ID NO: 426 - DNA, 75 bp, Homo sapiens
ggcgccgtggcttagctggttaaagcgcctgtctagtaaacaggagatcctgggttcgaatcccagcggtgcctg

SEQ ID NO: 427 - DNA, 73 bp, Homo sapiens
cgacctcgtggcgcaacggtagcgcgtctgactccagatcagaaggttgcgtgttcaaatcacgtcggggtca

SEQ ID NO: 428 - DNA, 83 bp, Homo sapiens
agacgaggtggccgagtggttaaggcgatggactgctaatccattgtgctctgcacgcgtgggttcgaatcccatcctcgtcg

SEQ ID NO: 429 - DNA, 75 bp, Homo sapiens
cggcgccgtggcttagttggttaaagcgcctgtctagtaaacaggagatcctgggttcgaatcccagcggtgcct

SEQ ID NO: 430 - DNA, 73 bp, Homo sapiens
ggcctcgtggcgcaacggtagcgcgtctgactccagatcagaaggttgcgtgttcaaatcacgtcggggtcaa

SEQ ID NO: 431 - DNA, 73 bp, Homo sapiens
agcgttggtggtatagtggtaagcatagctgccttccaagcagttgacccgggttcgattcccggccaacgca

SEQ ID NO: 432 - DNA, 73 bp, Homo sapiens
tcctcgttagtatagtggtgagtatccccgcctgtcacgcgggagaccggggttcgattccccgacggggaga

SEQ ID NO: 433 - DNA, 73 bp, Homo sapiens
ggctcgttggtctaggggtatgattctcgcttcgggtgcgagaggtcccgggttcaaatcccggacgagccct

SEQ ID NO: 434 - DNA, 75 bp, Homo sapiens
ggcgccgtggcttagttggttaaagcgcctgtctagtaaacaggagatcctgggttcgaatcccagcggtgcctt

SEQ ID NO: 435 - DNA, 83 bp, Homo sapiens
gtagtcgtggccgagtggttaaggcgatggactagaaatccattggggtctccccgcgcaggttcgaatcctgccgactacgt

SEQ ID NO: 436 - DNA, 73 bp, Homo sapiens
tgacctcgtggcgcaatggtagcgcgtctgactccagatcagaaggttgcgtgttcaagtcacgtcggggtca

SEQ ID NO: 437 - DNA, 73 bp, Homo sapiens
aggcgcggtggccaagtggtaaggcgtcggtctcgtaaaccgaagatcgcgggttcgaaccccgtccgtgcct

SEQ ID NO: 438 - DNA, 73 bp, Homo sapiens
agggggtatagctcagtggtagagcatttgactgcagatcaagaggtccccggttcaaatccgggtgccccct

SEQ ID NO: 439 - DNA, 73 bp, Homo sapiens
gggggtatagctcagtggtagagcatttgactgcagatcaagaggtccctggttcaaatccgggtgccccctc

SEQ ID NO: 440 - DNA, 73 bp, Homo sapiens
gggggtatagctcagtggtagagcatttgactgcagatcaagaggtccccggttcaaatccgggtgccccctc

SEQ ID NO: 441 - DNA, 73 bp, Homo sapiens
aggtcccatggtgtaatggttagcactctggactttgaatccagcgatccgagttcaaatctcggtgggacct

SEQ ID NO: 442 - DNA, 74 bp, Homo sapiens
gacccagtggcctaatggataaggcatcagcctccggagctggggattgtgggttcgagtcccatctgggtcgc

SEQ ID NO: 443 - DNA, 74 bp, Homo sapiens
agccccagtggcctaatggataaggcactggcctcctaagccagggattgtgggttcgagtcccacctggggta

SEQ ID NO: 444 - DNA, 74 bp, Homo sapiens
gccccagtggcctaatggataaggcactggcctcctaagccagggattgtgggttcgagtcccacctggggtgt

SEQ ID NO: 445 - DNA, 74 bp, Homo sapiens
agaccgcgtggcctaatggataaggcgtctgacttcggatcagaagattgagggttcgagtcccttcgtggtcg

SEQ ID NO: 446 - DNA, 75 bp, Homo sapiens
cgtctctgtggcgcaatcggttagcgcgttcggctgttaaccgaaaggttggtggttcgagcccacccagggacg

SEQ ID NO: 447 - DNA, 73 bp, Homo sapiens
ggcgttggtggtatagtggttagcatagctgccttccaagcagttgacccgggttcgattcccggccaacgca

SEQ ID NO: 448 - DNA, 74 bp, Homo sapiens
gtttccgtagtgtagcggttatcacattcgcctcacacgcgaaaggtccccggttcgatcccgggcggaaacag

SEQ ID NO: 449 - DNA, 75 bp, Homo sapiens
tggcgccgtggcttagttggttaaagcgcctgtctagtaaacaggagatcctgggttcgaatcccagcggtgcct

SEQ ID NO: 450 - DNA, 94 bp, Homo sapiens
gctccagtggcgcaatcggttagcgcgcggtacttatatgacagtgcgagcggagcaatgccgaggttgtgagttcgatcctcacctggagcac

SEQ ID NO: 451 - DNA, 72 bp, Homo sapiens
gcatgggtggttcagtggtagaattctcgcctgccacgcgggaggcccgggttcgattcccggcccatgcag

SEQ ID NO: 452 - DNA, 18 bp, Artificial Sequence
  source: /note=“Description of Artificial Sequence Synthetic oligonucleotide”
aaaatataaatatatttc

SEQ ID NO: 453 - DNA, 5 bp, Artificial Sequence
  source: /note=“Description of Artificial Sequence Synthetic oligonucleotide”
aagct

SEQ ID NO: 454 - DNA, 5 bp, Artificial Sequence
  source: /note=“Description of Artificial Sequence Synthetic oligonucleotide”
aagtt

SEQ ID NO: 455 - DNA, 15 bp, Artificial Sequence
  source: /note=“Description of Artificial Sequence Synthetic oligonucleotide”
aattcttcggaatgt

SEQ ID NO: 456 - DNA, 3 bp, Artificial Sequence
  source: /note=“Description of Artificial Sequence Synthetic oligonucleotide”
aga

SEQ ID NO: 457 - DNA, 5 bp, Artificial Sequence
  source: /note=“Description of Artificial Sequence Synthetic oligonucleotide”
agtcc

SEQ ID NO: 458 - DNA, 5 bp, Artificial Sequence
  source: /note=“Description of Artificial Sequence Synthetic oligonucleotide”
caacc

SEQ ID NO: 459 - DNA, 5 bp, Artificial Sequence
  source: /note=“Description of Artificial Sequence Synthetic oligonucleotide”
caatc

SEQ ID NO: 460 - DNA, 4 bp, Artificial Sequence
  source: /note=“Description of Artificial Sequence Synthetic oligonucleotide”
cagc

SEQ ID NO: 461 - DNA, 20 bp, Artificial Sequence
  source: /note=“Description of Artificial Sequence Synthetic oligonucleotide”
caggcgggttctgcccgcgc

SEQ ID NO: 462 - DNA, 18 bp, Artificial Sequence
  source: /note=“Description of Artificial Sequence Synthetic oligonucleotide”
catacctgcaagggtatc

SEQ ID NO: 463 - DNA, 15 bp, Artificial Sequence
  source: /note=“Description of Artificial Sequence Synthetic oligonucleotide”
cgaccgcaaggttgt

SEQ ID NO: 464 - DNA, 15 bp, Artificial Sequence
  source: /note=“Description of Artificial Sequence Synthetic oligonucleotide”
cgaccttgcggtcat

SEQ ID NO: 465 - DNA, 18 bp, Artificial Sequence
  source: /note=“Description of Artificial Sequence Synthetic oligonucleotide”
cgatgctaatcacatcgt

SEQ ID NO: 466 - DNA, 15 bp, Artificial Sequence
  source: /note=“Description of Artificial Sequence Synthetic oligonucleotide”
cgatggtgacatcat

SEQ ID NO: 467 - DNA, 16 bp, Artificial Sequence
  source: /note=“Description of Artificial Sequence Synthetic oligonucleotide”
cgatggtttacatcgt

SEQ ID NO: 468 - DNA, 13 bp, Artificial Sequence
  source: /note=“Description of Artificial Sequence Synthetic oligonucleotide”
cgccgtaaggtgt

SEQ ID NO: 469 - DNA, 12 bp, Artificial Sequence
  source: /note=“Description of Artificial Sequence Synthetic oligonucleotide”
cgccttaggtgt

SEQ ID NO: 470 - DNA, 15 bp, Artificial Sequence
  source: /note=“Description of Artificial Sequence Synthetic oligonucleotide”
cgcctttcgacgcgt

SEQ ID NO: 471 - DNA, 13 bp, Artificial Sequence
  source: /note=“Description of Artificial Sequence Synthetic oligonucleotide”
cgcttcacggcgt

SEQ ID NO: 472 - DNA, 15 bp, Artificial Sequence
  source: /note=“Description of Artificial Sequence Synthetic oligonucleotide”
cggcagcaatgctgt

SEQ ID NO: 473 - DNA, 13 bp, Artificial Sequence
  source: /note=“Description of Artificial Sequence Synthetic oligonucleotide”
cggctccgccttc

SEQ ID NO: 474 - DNA, 16 bp, Artificial Sequence
  source: /note=“Description of Artificial Sequence Synthetic oligonucleotide”
cgggtatcacagggtc

SEQ ID NO: 475 - DNA, 17 bp, Artificial Sequence
  source: /note=“Description of Artificial Sequence Synthetic oligonucleotide”
cggtgcgcaagcgctgt

SEQ ID NO: 476 - DNA, 18 bp, Artificial Sequence
  source: /note=“Description of Artificial Sequence Synthetic oligonucleotide”
cgtacgggtgaccgtacc

SEQ ID NO: 477 - DNA, 13 bp, Artificial Sequence
  source: /note=“Description of Artificial Sequence Synthetic oligonucleotide”
cgtcaaagacttc

SEQ ID NO: 478 - DNA, 13 bp, Artificial Sequence
  source: /note=“Description of Artificial Sequence Synthetic oligonucleotide”
cgtcgtaagactt

SEQ ID NO: 479 - DNA, 14 bp, Artificial Sequence
  source: /note=“Description of Artificial Sequence Synthetic oligonucleotide”
cgttgaataaacgt

SEQ ID NO: 480 - DNA, 5 bp, Artificial Sequence
  source: /note=“Description of Artificial Sequence Synthetic oligonucleotide”
ctgtc

SEQ ID NO: 481 - DNA, 4 bp, Artificial Sequence
  source: /note=“Description of Artificial Sequence Synthetic oligonucleotide”
ggcc

SEQ ID NO: 482 - DNA, 7 bp, Artificial Sequence
  source: /note=“Description of Artificial Sequence Synthetic oligonucleotide”
ggggatt

SEQ ID NO: 483 - DNA, 4 bp, Artificial Sequence
  source: /note=“Description of Artificial Sequence Synthetic oligonucleotide”
ggtc

SEQ ID NO: 484 - DNA, 5 bp, Artificial Sequence
  source: /note=“Description of Artificial Sequence Synthetic oligonucleotide”
ggttt

SEQ ID NO: 485 - DNA, 4 bp, Artificial Sequence
  source: /note=“Description of Artificial Sequence Synthetic oligonucleotide”
gtag

SEQ ID NO: 486 - DNA, 19 bp, Artificial Sequence
  source: /note=“Description of Artificial Sequence Synthetic oligonucleotide”
taactagatactttcagat

SEQ ID NO: 487 - DNA, 15 bp, Artificial Sequence
  source: /note=“Description of Artificial Sequence Synthetic oligonucleotide”
tactcgtatgggtgc

SEQ ID NO: 488 - DNA, 13 bp, Artificial Sequence
  source: /note=“Description of Artificial Sequence Synthetic oligonucleotide”
tactttgcggtgt

SEQ ID NO: 489 - DNA, 19 bp, Artificial Sequence
  source: /note=“Description of Artificial Sequence Synthetic oligonucleotide”
taggcgagtaacatcgtgc

SEQ ID NO: 490 - DNA, 19 bp, Artificial Sequence
  source: /note=“Description of Artificial Sequence Synthetic oligonucleotide”
taggcgtgaatagcgcctc

SEQ ID NO: 491 - DNA, 19 bp, Artificial Sequence
  source: /note=“Description of Artificial Sequence Synthetic oligonucleotide”
taggtcgcgagagcggcgc

SEQ ID NO: 492 - DNA, 19 bp, Artificial Sequence
  source: /note=“Description of Artificial Sequence Synthetic oligonucleotide”
taggtcgcgtaagcggcgc

SEQ ID NO: 493 - DNA, 17 bp, Artificial Sequence
  source: /note=“Description of Artificial Sequence Synthetic oligonucleotide”
taggtggttatccacgc

SEQ ID NO: 494 - DNA, 5 bp, Artificial Sequence
  source: /note=“Description of Artificial Sequence Synthetic oligonucleotide”
tagtc

SEQ ID NO: 495 - DNA, 5 bp, Artificial Sequence
  source: /note=“Description of Artificial Sequence Synthetic oligonucleotide”
tagtt

SEQ ID NO: 496 - DNA, 18 bp, Artificial Sequence
  source: /note=“Description of Artificial Sequence Synthetic oligonucleotide”
tatacgtgaaagcgtatc

SEQ ID NO: 497 - DNA, 21 bp, Artificial Sequence
  source: /note=“Description of Artificial Sequence Synthetic oligonucleotide”
tatagggtcaaaaactctatc

SEQ ID NO: 498 - DNA, 20 bp, Artificial Sequence
  source: /note=“Description of Artificial Sequence Synthetic oligonucleotide”
tatgcagaaatacctgcatc

SEQ ID NO: 499 - DNA, 15 bp, Artificial Sequence
  source: /note=“Description of Artificial Sequence Synthetic oligonucleotide”
tccccatacgggggc

SEQ ID NO: 500 - DNA, 14 bp, Artificial Sequence
  source: /note=“Description of Artificial Sequence Synthetic oligonucleotide”
tcccgaaggggttc

SEQ ID NO: 501 - DNA, 15 bp, Artificial Sequence
  source: /note=“Description of Artificial Sequence Synthetic oligonucleotide”
tctacgtatgtgggc

SEQ ID NO: 502 - DNA, 14 bp, Artificial Sequence
  source: /note=“Description of Artificial Sequence Synthetic oligonucleotide”
tctcataggagttc

SEQ ID NO: 503 - DNA, 14 bp, Artificial Sequence
  source: /note=“Description of Artificial Sequence Synthetic oligonucleotide”
tctcctctggaggc

SEQ ID NO: 504 - DNA, 15 bp, Artificial Sequence
  source: /note=“Description of Artificial Sequence Synthetic oligonucleotide”
tcttagcaataaggt

SEQ ID NO: 505 - DNA, 14 bp, Artificial Sequence
  source: /note=“Description of Artificial Sequence Synthetic oligonucleotide”
tcttgtaggagttc

SEQ ID NO: 506 - DNA, 15 bp, Artificial Sequence
  source: /note=“Description of Artificial Sequence Synthetic oligonucleotide”
tgaacgtaagttcgc

SEQ ID NO: 507 - DNA, 16 bp, Artificial Sequence
  source: /note=“Description of Artificial Sequence Synthetic oligonucleotide”
tgaactgcgaggttcc

SEQ ID NO: 508 - DNA, 4 bp, Artificial Sequence
  source: /note=“Description of Artificial Sequence Synthetic oligonucleotide”
tgac

SEQ ID NO: 509 - DNA, 15 bp, Artificial Sequence
  source: /note=“Description of Artificial Sequence Synthetic oligonucleotide”
tgaccgaaaggtcgt

SEQ ID NO: 510 - DNA, 15 bp, Artificial Sequence
  source: /note=“Description of Artificial Sequence Synthetic oligonucleotide”
tgaccgcaaggtcgt

SEQ ID NO: 511 - DNA, 14 bp, Artificial Sequence
  source: /note=“Description of Artificial Sequence Synthetic oligonucleotide”
tgagctctgctctc

SEQ ID NO: 512 - DNA, 18 bp, Artificial Sequence
  source: /note=“Description of Artificial Sequence Synthetic oligonucleotide”
tgaggcctcacggcctac

SEQ ID NO: 513 - DNA, 15 bp, Artificial Sequence
  source: /note=“Description of Artificial Sequence Synthetic oligonucleotide”
tgagggcaacttcgt

SEQ ID NO: 514 - DNA, 16 bp, Artificial Sequence
  source: /note=“Description of Artificial Sequence Synthetic oligonucleotide”
tgagggtcatacctcc

SEQ ID NO: 515 - DNA, 18 bp, Artificial Sequence
  source: /note=“Description of Artificial Sequence Synthetic oligonucleotide”
tgagggtgcaaatcctcc

SEQ ID NO: 516 - DNA, 13 bp, Artificial Sequence
  source: /note=“Description of Artificial Sequence Synthetic oligonucleotide”
tgccgaaaggcgt

SEQ ID NO: 517 - DNA, 13 bp, Artificial Sequence
  source: /note=“Description of Artificial Sequence Synthetic oligonucleotide”
tgccgtaaggcgt

SEQ ID NO: 518 - DNA, 14 bp, Artificial Sequence
  source: /note=“Description of Artificial Sequence Synthetic oligonucleotide”
tgcggtctccgcgc

SEQ ID NO: 519 - DNA, 11 bp, Artificial Sequence
  source: /note=“Description of Artificial Sequence Synthetic oligonucleotide”
tgctagagcat

SEQ ID NO: 520 - DNA, 16 bp, Artificial Sequence
  source: /note=“Description of Artificial Sequence Synthetic oligonucleotide”
tgctcgtatagagctc

SEQ ID NO: 521 - DNA, 15 bp, Artificial Sequence
  source: /note=“Description of Artificial Sequence Synthetic oligonucleotide”
tggacaattgtctgc

SEQ ID NO: 522 - DNA, 15 bp, Artificial Sequence
  source: /note=“Description of Artificial Sequence Synthetic oligonucleotide”
tggacagatgtccgt

SEQ ID NO: 523 - DNA, 15 bp, Artificial Sequence
  source: /note=“Description of Artificial Sequence Synthetic oligonucleotide”
tggacaggtgtccgc

SEQ ID NO: 524 - DNA, 15 bp, Artificial Sequence
  source: /note=“Description of Artificial Sequence Synthetic oligonucleotide”
tggacggttgtccgc

SEQ ID NO: 525 - DNA, 13 bp, Artificial Sequence
  source: /note=“Description of Artificial Sequence Synthetic oligonucleotide”
tggacttgtggtc

SEQ ID NO: 526 - DNA, 16 bp, Artificial Sequence
  source: /note=“Description of Artificial Sequence Synthetic oligonucleotide”
tggagattctctccgc

SEQ ID NO: 527 - DNA, 14 bp, Artificial Sequence
  source: /note=“Description of Artificial Sequence Synthetic oligonucleotide”
tggcataggcctgc

SEQ ID NO: 528 - DNA, 14 bp, Artificial Sequence
  source: /note=“Description of Artificial Sequence Synthetic oligonucleotide”
tggcttatgtctac

SEQ ID NO: 529 - DNA, 16 bp, Artificial Sequence
  source: /note=“Description of Artificial Sequence Synthetic oligonucleotide”
tgggagttaatcccgt

SEQ ID NO: 530 - DNA, 14 bp, Artificial Sequence
  source: /note=“Description of Artificial Sequence Synthetic oligonucleotide”
tgggatcttcccgc

SEQ ID NO: 531 - DNA, 16 bp, Artificial Sequence
  source: /note=“Description of Artificial Sequence Synthetic oligonucleotide”
tgggcagaaatgtctc

SEQ ID NO: 532 - DNA, 15 bp, Artificial Sequence
  source: /note=“Description of Artificial Sequence Synthetic oligonucleotide”
tgggcgttcgcccgc

SEQ ID NO: 533 - DNA, 14 bp, Artificial Sequence
  source: /note=“Description of Artificial Sequence Synthetic oligonucleotide”
tgggcttcgcccgc

SEQ ID NO: 534 - DNA, 16 bp, Artificial Sequence
  source: /note=“Description of Artificial Sequence Synthetic oligonucleotide”
tgggggataaccccgt

SEQ ID NO: 535 - DNA, 15 bp, Artificial Sequence
  source: /note=“Description of Artificial Sequence Synthetic oligonucleotide”
tgggggtttccccgt

SEQ ID NO: 536 - DNA, 4 bp, Artificial Sequence
  source: /note=“Description of Artificial Sequence Synthetic oligonucleotide”
tggt

SEQ ID NO: 537 - DNA, 15 bp, Artificial Sequence
  source: /note=“Description of Artificial Sequence Synthetic oligonucleotide”
tggtggcaacaccgt

SEQ ID NO: 538 - DNA, 14 bp, Artificial Sequence
  source: /note=“Description of Artificial Sequence Synthetic oligonucleotide”
tggtttatagccgt

SEQ ID NO: 539 - DNA, 19 bp, Artificial Sequence
  source: /note=“Description of Artificial Sequence Synthetic oligonucleotide”
tgtacggtaataccgtacc

SEQ ID NO: 540 - DNA, 15 bp, Artificial Sequence
  source: /note=“Description of Artificial Sequence Synthetic oligonucleotide”
tgtccgcaaggacgt

SEQ ID NO: 541 - DNA, 15 bp, Artificial Sequence
  source: /note=“Description of Artificial Sequence Synthetic oligonucleotide”
tgtcctaacggacgt

SEQ ID NO: 542 - DNA, 18 bp, Artificial Sequence
  source: /note=“Description of Artificial Sequence Synthetic oligonucleotide”
tgtcctattaacggacgt

SEQ ID NO: 543 - DNA, 16 bp, Artificial Sequence
  source: /note=“Description of Artificial Sequence Synthetic oligonucleotide”
tgtccttcacgggcgt

SEQ ID NO: 544 - DNA, 13 bp, Artificial Sequence
  source: /note=“Description of Artificial Sequence Synthetic oligonucleotide”
tgtcttaggacgt

SEQ ID NO: 545 - DNA, 18 bp, Artificial Sequence
  source: /note=“Description of Artificial Sequence Synthetic oligonucleotide”
tgtgcgttaacgcgtacc

SEQ ID NO: 546 - DNA, 16 bp, Artificial Sequence
  source: /note=“Description of Artificial Sequence Synthetic oligonucleotide”
tgtgtcgcaaggcacc

SEQ ID NO: 547 - DNA, 15 bp, Artificial Sequence
  source: /note=“Description of Artificial Sequence Synthetic oligonucleotide”
tgttcgtaaggactt

SEQ ID NO: 548 - DNA, 16 bp, Artificial Sequence
  source: /note=“Description of Artificial Sequence Synthetic oligonucleotide”
ttcacagaaatgtgtc

SEQ ID NO: 549 - DNA, 14 bp, Artificial Sequence
  source: /note=“Description of Artificial Sequence Synthetic oligonucleotide”
ttccctcgtggagt

SEQ ID NO: 550 - DNA, 14 bp, Artificial Sequence
  source: /note=“Description of Artificial Sequence Synthetic oligonucleotide”
ttccctctgggagc

SEQ ID NO: 551 - DNA, 14 bp, Artificial Sequence
  source: /note=“Description of Artificial Sequence Synthetic oligonucleotide”
ttcccttgtggatc

SEQ ID NO: 552 - DNA, 13 bp, Artificial Sequence
  source: /note=“Description of Artificial Sequence Synthetic oligonucleotide”
ttccttcgggagc

SEQ ID NO: 553 - DNA, 15 bp, Artificial Sequence
  source: /note=“Description of Artificial Sequence Synthetic oligonucleotide”
ttctagcaatagagt

SEQ ID NO: 554 - DNA, 16 bp, Artificial Sequence
  source: /note=“Description of Artificial Sequence Synthetic oligonucleotide”
ttctccactggggagc

SEQ ID NO: 555 - DNA, 15 bp, Artificial Sequence
  source: /note=“Description of Artificial Sequence Synthetic oligonucleotide”
ttctcgagagggagc

SEQ ID NO: 556 - DNA, 15 bp, Artificial Sequence
  source: /note=“Description of Artificial Sequence Synthetic oligonucleotide”
ttctcgtatgagagc

SEQ ID NO: 557 - DNA, 19 bp, Artificial Sequence
  source: /note=“Description of Artificial Sequence Synthetic oligonucleotide”
tttaaggttttcccttaac

SEQ ID NO: 558 - DNA, 14 bp, Artificial Sequence
  source: /note=“Description of Artificial Sequence Synthetic oligonucleotide”
tttcattgtggagt

SEQ ID NO: 559 - DNA, 14 bp, Artificial Sequence
  source: /note=“Description of Artificial Sequence Synthetic oligonucleotide”
tttcgaaggaatcc

SEQ ID NO: 560 - DNA, 13 bp, Artificial Sequence
  source: /note=“Description of Artificial Sequence Synthetic oligonucleotide”
tttcttcggaagc

SEQ ID NO: 561 - DNA, 16 bp, Artificial Sequence
  source: /note=“Description of Artificial Sequence Synthetic oligonucleotide”
tttggggcaactcaac

SEQ ID NO: 562 - RNA, 342 nt, Artificial Sequence
  source: /note=“Description of Artificial Sequence Synthetic polynucleotide”
  modified_base (5)..(6): a, c, u, or g
  modified_base (15)..(16): a, c, u, or g
  modified_base (21)..(21): a, c, u, or g
  modified_base (30)..(32): a, c, u, or g
  modified_base (34)..(34): a, c, u, or g
  modified_base (37)..(37): a, c, u, or g
  modified_base (41)..(45): a, c, u, or g
  misc_feature (47)..(317): /note=“This region may encompass 1-271 nucleotides”
  modified_base (47)..(320): a, c, u, or g
  modified_base (328)..(329): a, c, u, or g
  modified_base (333)..(334): a, c, u, or g
  modified_base (336)..(337): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rkssnndurghbyannyugrndgwdvdydnnnbnhbnryrnnnnndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrrduyrannyybnnhnnbwccd

SEQ ID NO: 563 - RNA, 341 nt, Artificial Sequence
  source: /note=“Description of Artificial Sequence Synthetic polynucleotide”
  modified_base (16)..(16): a, c, u, or g
  modified_base (30)..(30): a, c, u, or g
  modified_base (46)..(316): a, c, u, or g
  misc_feature (46)..(316): /note=“This region may encompass 1-271 nucleotides”
  modified_base (319)..(319): a, c, u, or g
  modified_base (328)..(328): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
rksgrwdkrghbyavnyggudgarvrydynhkywbhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgducrayncyydvhhyywccd

SEQ ID NO: 564 - RNA, 341 nt, Artificial Sequence
  source: /note=“Description of Artificial Sequence Synthetic polynucleotide”
  modified_base (16)..(16): a, c, u, or g
  modified_base (30)..(30): a, c, u, or g
  modified_base (46)..(316): a, c, u, or g
  misc_feature (46)..(316): /note=“This region may encompass 1-271 nucleotides”
  modified_base (319)..(319): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ggggrwdurghbyavnyggudgarvrydynhkywkhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgducraybccydvhhyyucca

SEQ ID NO: 565 - RNA, 343 nt, Artificial Sequence
  source: /note=“Description of Artificial Sequence Synthetic polynucleotide”
  modified_base (2)..(8): a, c, u, or g
  modified_base (12)..(13): a, c, u, or g
  modified_base (17)..(17): a, c, u, or g
  modified_base (22)..(24): a, c, u, or g
  modified_base (26)..(27): a, c, u, or g
  modified_base (30)..(35): a, c, u, or g
  modified_base (38)..(38): a, c, u, or g
  modified_base (43)..(43): a, c, u, or g
  modified_base (45)..(322): a, c, u, or g
  misc_feature (48)..(318): /note=“This region may encompass 1-271 nucleotides”
  modified_base (329)..(329): a, c, u, or g
  modified_base (333)..(342): a, c, u, or g
  variation (1)..(343): /replace=“ ”
  misc_feature (1)..(343): /note=“Variant nucleotides given in the sequence have no preference with respect to those in the annotations for variant positions”
  source: /note=“See specification as filed for detailed description of substitutions and preferred embodiments”
gnnnnnnnkvgnnhddnhwrrnnnrnnvynnnnnnbunckrhnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnguuyranyybnnnnnnnnnnd

SEQ ID NO: 566 - RNA, 342 nt, Artificial Sequence
  source: /note=“Description of Artificial Sequence Synthetic polynucleotide”
  modified_base (3)..(3): a, c, u, or g
  modified_base (6)..(7): a, c, u, or g
  modified_base (13)..(13): a, c, u, or g
  modified_base (26)..(26): a, c, u, or g
  modified_base (32)..(32): a, c, u, or g
  modified_base (37)..(37): a, c, u, or g
  modified_base (44)..(44): a, c, u, or g
  modified_base (47)..(317): a, c, u, or g
  misc_feature (47)..(317): /note=“This region may encompass 1-271 nucleotides”
  modified_base (328)..(328): a, c, u, or g
  modified_base (336)..(338): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
grnvbnnvkbgbnydrwurgwygarnrydyynsvyunckrmkbnrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshvgguuyranuycbdbynnnbnhr

SEQ ID NO: 567 - RNA, 342 nt, Artificial Sequence
  source: /note=“Description of Artificial Sequence Synthetic polynucleotide”
  modified_base (3)..(3): a, c, u, or g
  modified_base (7)..(7): a, c, u, or g
  modified_base (47)..(317): a, c, u, or g
  misc_feature (47)..(317): /note=“This region may encompass 1-271 nucleotides”
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
grnvyvnskkgssydawuggaygarsgyryykgmyuhckrakymrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshrgguuygavuycydbynbdbnyg

SEQ ID NO: 568 - RNA, 341 nt, Artificial Sequence
  source: /note=“Description of Artificial Sequence Synthetic polynucleotide”
  modified_base (2)..(2): a, c, u, or g
  modified_base (4)..(6): a, c, u, or g
  modified_base (12)..(12): a, c, u, or g
  modified_base (17)..(17): a, c, u, or g
  modified_base (25)..(25): a, c, u, or g
  modified_base (28)..(30): a, c, u, or g
  modified_base (43)..(316): a, c, u, or g
  misc_feature (46)..(316): /note=“This region may encompass 1-271 nucleotides”
  modified_base (318)..(319): a, c, u, or g
  modified_base (327)..(327): a, c, u, or g
  modified_base (331)..(332): a, c, u, or g
  modified_base (334)..(337): a, c, u, or g
  modified_base (339)..(340): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dnynnndurghnhvrynggbhurdnrynnnbsryyruuwahbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgukhvwnhcynnbnnnndnnr

SEQ ID NO: 569 - RNA, 341 nt, Artificial Sequence
  source: /note=“Description of Artificial Sequence Synthetic polynucleotide”
  modified_base (17)..(17): a, c, u, or g
  modified_base (28)..(28): a, c, u, or g
  modified_base (46)..(316): a, c, u, or g
  misc_feature (46)..(316): /note=“This region may encompass 1-271 nucleotides”
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ruyucygurghryvrunggbyuarhgcnuuysryyruuwahbddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgurgukhvwdmcyayysvrrrrbr

SEQ ID NO: 570 - RNA, 341 nt, Artificial Sequence
  source: /note=“Description of Artificial Sequence Synthetic polynucleotide”
  modified_base (46)..(316): a, c, u, or g
  misc_feature (46)..(316): /note=“This region may encompass 1-271 nucleotides”
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ruyucygurghryvrubggbyuaghgcbuuysryyruuaahyddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgurgukhvwdmcyayysvrrrryg

SEQ ID NO: 571 - RNA, 342 nt, Artificial Sequence
  source: /note=“Description of Artificial Sequence Synthetic polynucleotide”
  modified_base (2)..(6): a, c, u, or g
  modified_base (12)..(12): a, c, u, or g
  modified_base (16)..(16): a, c, u, or g
  modified_base (22)..(22): a, c, u, or g
  modified_base (26)..(26): a, c, u, or g
  modified_base (29)..(29): a, c, u, or g
  modified_base (31)..(32): a, c, u, or g
  modified_base (44)..(44): a, c, u, or g
  misc_feature (47)..(317): /note=“This region may encompass 1-271 nucleotides”
  modified_base (47)..(319): a, c, u, or g
  modified_base (328)..(328): a, c, u, or g
  modified_base (333)..(334): a, c, u, or g
  modified_base (336)..(339): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
knnnnndyrrynhrrnyygrhnumdnrynbnnvhyurucrhdbnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsrgyubrhnyyysnnhnnnnbmr

SEQ ID NO: 572 - RNA, 339 nt, Artificial Sequence
  source: /note=“Description of Artificial Sequence Synthetic polynucleotide”
  modified_base (2)..(2): a, c, u, or g
  modified_base (6)..(6): a, c, u, or g
  modified_base (44)..(314): a, c, u, or g
  misc_feature (44)..(314): /note=“This region may encompass 1-271 nucleotides”
  modified_base (333)..(333): a, c, u, or g
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
knsbbnwyarywyrguggukmgwrubysyryyurucaygbrsrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnykgrgyubrhkyycchrwnvvksmr

SEQ ID NO: 573 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
umcuyruyaguaurguggukmguruyycygycugucaygyggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyggrguuyrmkyyccyrasrrggag

SEQ ID NO: 574 - RNA, 342 nt, Artificial Sequence
  source: /note=“Description of Artificial Sequence Synthetic polynucleotide”
  modified_base (3)..(6): a, c, u, or g
  modified_base (12)..(13): a, c, u, or g
  modified_base (16)..(16): a, c, u, or g
  modified_base (24)..(24): a, c, u, or g
  modified_base (26)..(26): a, c, u, or g
  modified_base (29)..(32): a, c, u, or g
  modified_base (34)..(34): a, c, u, or g
  modified_base (42)..(42): a, c, u, or g
  modified_base (44)..(319): a, c, u, or g
  misc_feature (47)..(317): /note=“This region may encompass 1-271 nucleotides”
  modified_base (328)..(328): a, c, u, or g
  modified_base (333)..(334): a, c, u, or g
  modified_base (336)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
gsnnnndurvynnavnkcgdbyynrnvbnnnnvnyyrcarmnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdruucranyyyvnnhnnnnnyy

SEQ ID NO: 575 - RNA, 340 nt, Artificial Sequence
  source: /note=“Description of Artificial Sequence Synthetic polynucleotide”
  modified_base (16)..(16): a, c, u, or g
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsgsryrkrgyubasnkgdkuaravyahuugrcyrcaraucmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyydruucraruyyvgkuryychyy

SEQ ID NO: 576 - RNA, 340 nt, Artificial Sequence
  source: /note=“Description of Artificial Sequence Synthetic polynucleotide”
  modified_base (16)..(16): a, c, u, or g
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gggsryrkrgyuyasnugrkuagagcayuugacugcaraucmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyyrruucraauyyvgkurcycmyy

SEQ ID NO: 577 - RNA, 341 nt, Artificial Sequence
  source: /note=“Description of Artificial Sequence Synthetic polynucleotide”
  modified_base (2)..(2): a, c, u, or g
  modified_base (4)..(6): a, c, u, or g
  modified_base (12)..(13): a, c, u, or g
  modified_base (16)..(16): a, c, u, or g
  modified_base (20)..(21): a, c, u, or g
  modified_base (24)..(24): a, c, u, or g
  modified_base (28)..(31): a, c, u, or g
  modified_base (33)..(33): a, c, u, or g
  modified_base (40)..(41): a, c, u, or g
  modified_base (43)..(319): a, c, u, or g
  misc_feature (46)..(316): /note=“This region may encompass 1-271 nucleotides”
  modified_base (327)..(327): a, c, u, or g
  modified_base (331)..(332): a, c, u, or g
  modified_base (335)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dnbnnnduvgbnnarnhggnnhanvvynnnnvnyubukrnnhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrruuyvanybynnbhnnnnnhd

SEQ ID NO: 578 - RNA, 340 nt, Artificial Sequence
  source: /note=“Description of Artificial Sequence Synthetic polynucleotide”
  modified_base (4)..(5): a, c, u, or g
  modified_base (12)..(12): a, c, u, or g
  modified_base (21)..(21): a, c, u, or g
  modified_base (27)..(28): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (335)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kdbnnyduggynbarkmggdnavvrynnhbvryubukrrbhvdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrruuyvadybyhnbhdnnvhmd

SEQ ID NO: 579 - RNA, 340 nt, Artificial Sequence
  source: /note=“Description of Artificial Sequence Synthetic polynucleotide”
  modified_base (4)..(4): a, c, u, or g
  modified_base (21)..(21): a, c, u, or g
  modified_base (27)..(28): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kdbnvyduggydbarumggknavvrynnwsrryubukrabhsdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrruuyvadysyhnbhdbnvhmk

SEQ ID NO: 580 - RNA, 342 nt, Artificial Sequence
  source: /note=“Description of Artificial Sequence Synthetic polynucleotide”
  modified_base (1)..(2): a, c, u, or g
  modified_base (5)..(6): a, c, u, or g
  modified_base (9)..(9): a, c, u, or g
  modified_base (12)..(12): a, c, u, or g
  modified_base (16)..(16): a, c, u, or g
  modified_base (20)..(21): a, c, u, or g
  modified_base (26)..(27): a, c, u, or g
  modified_base (29)..(33): a, c, u, or g
  modified_base (41)..(41): a, c, u, or g
  modified_base (44)..(318): a, c, u, or g
  misc_feature (47)..(317): /note=“This region may encompass 1-271 nucleotides”
  modified_base (320)..(321): a, c, u, or g
  modified_base (328)..(328): a, c, u, or g
  modified_base (333)..(336): a, c, u, or g
  modified_base (340)..(341): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nnbbnndunrwnhrdnhygnnbhrdnnynnnnnvyuyusrnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnryubrwnyyhbnnnndvvnnd

SEQ ID NO: 581 - RNA, 340 nt, Artificial Sequence
  source: /note=“Description of Artificial Sequence Synthetic polynucleotide”
  modified_base (1)..(2): a, c, u, or g
  modified_base (5)..(5): a, c, u, or g
  modified_base (12)..(12): a, c, u, or g
  modified_base (20)..(20): a, c, u, or g
  modified_base (29)..(29): a, c, u, or g
  modified_base (31)..(31): a, c, u, or g
  modified_base (39)..(39): a, c, u, or g
  modified_base (43)..(43): a, c, u, or g
  misc_feature (45)..(315): /note=“This region may encompass 1-271 nucleotides”
  modified_base (45)..(316): a, c, u, or g
  modified_base (319)..(319): a, c, u, or g
  modified_base (326)..(326): a, c, u, or g
  modified_base (334)..(334): a, c, u, or g
  modified_base (338)..(338): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
nnbynbdyrgunurdyagbnkrrvvywbnbnvyuyusanbvhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnydnryubrwnychbrkbndvvnvd

SEQ ID NO: 582 - RNA, 339 nt, Artificial Sequence
  source: /note=“Description of Artificial Sequence Synthetic polynucleotide”
  modified_base (1)..(1): a, c, u, or g
  modified_base (28)..(28): a, c, u, or g
  modified_base (30)..(30): a, c, u, or g
  modified_base (42)..(42): a, c, u, or g
  modified_base (44)..(314): a, c, u, or g
  misc_feature (44)..(314): /note=“This region may encompass 1-271 nucleotides”
  modified_base (318)..(318): a, c, u, or g
  modified_base (333)..(333): a, c, u, or g
  variation (1)..(339): /replace=“ ”
  misc_feature (1)..(339): /note=“Variant nucleotides given in the sequence have nopreference with respect to those in the annotations for variant positions”
  source: /note=“See specification as filed for detailed description of substitutions and preferred embodiments”
ncsydbruggusuakygshkrrsaywbnknsyuyucahbvmndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbyrnryuyrwyucyyrgbnwrsraw

SEQ ID NO: 583 - RNA, 342 nt, Artificial Sequence
  source: /note=“Description of Artificial Sequence Synthetic polynucleotide”
  modified_base (4)..(7): a, c, u, or g
  modified_base (12)..(12): a, c, u, or g
  modified_base (16)..(16): a, c, u, or g
  modified_base (21)..(22): a, c, u, or g
  modified_base (26)..(26): a, c, u, or g
  modified_base (29)..(34): a, c, u, or g
  modified_base (41)..(319): a, c, u, or g
  misc_feature (47)..(317): /note=“This region may encompass 1-271 nucleotides”
  modified_base (328)..(328): a, c, u, or g
  modified_base (333)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbvnnnnuvrynyrrnyubdnnuadnrynnnnnnyybccvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvvsubbrwnhcbbnnnnnnbbyw

SEQ ID NO: 584 - RNA, 340 nt, Artificial Sequence
  source: /note=“Description of Artificial Sequence Synthetic polynucleotide”
  modified_base (5)..(5): a, c, u, or g
  modified_base (28)..(28): a, c, u, or g
  modified_base (39)..(40): a, c, u, or g
  modified_base (42)..(42): a, c, u, or g
  modified_base (45)..(315): a, c, u, or g
  misc_feature (45)..(315): /note=“This region may encompass 1-271 nucleotides”
  modified_base (317)..(317): a, c, u, or g
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gbvbnsvurrudyrrdcggkdadmaudnhhrhyubccannynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnrgsuubrwuuccyksbsnvygca

SEQ ID NO: 585 - RNA, 340 nt, Artificial Sequence
  source: /note=“Description of Artificial Sequence Synthetic polynucleotide”
  modified_base (5)..(5): a, c, u, or g
  modified_base (28)..(28): a, c, u, or g
  modified_base (39)..(39): a, c, u, or g
  modified_base (42)..(42): a, c, u, or g
  modified_base (45)..(315): a, c, u, or g
  misc_feature (45)..(315): /note=“This region may encompass 1-271 nucleotides”
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gyvynsvurrudyrrwcggkkadmaudnhhrhyubccandynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbvrgsuuyrwuuccyksbsnvygca

SEQ ID NO: 586 - RNA, 342 nt, Artificial Sequence
  source: /note=“Description of Artificial Sequence Synthetic polynucleotide”
  modified_base (4)..(7): a, c, u, or g
  modified_base (13)..(13): a, c, u, or g
  modified_base (25)..(26): a, c, u, or g
  modified_base (29)..(31): a, c, u, or g
  modified_base (34)..(34): a, c, u, or g
  modified_base (42)..(42): a, c, u, or g
  modified_base (45)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
kgynnnnduvgbnhagbyugghyannvynnndbnyugugrhnvhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgyucranycynnbhnnnvrym

SEQ ID NO: 587 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gchrugaybguayagkgguuasyacucugygyuguggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwyggyucraaucygagucaygrca

SEQ ID NO: 588 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gcmrugayyguauagugguuagyacucugygyuguggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwygguucraaucygagucaygrca

SEQ ID NO: 589 - RNA, 342 nt, Artificial Sequence
  source: /note=“Description of Artificial Sequence Synthetic polynucleotide”
  modified_base (1)..(1): a, c, u, or g
  modified_base (4)..(5): a, c, u, or g
  modified_base (7)..(7): a, c, u, or g
  modified_base (17)..(17): a, c, u, or g
  modified_base (31)..(32): a, c, u, or g
  modified_base (34)..(34): a, c, u, or g
  modified_base (37)..(37): a, c, u, or g
  modified_base (42)..(320): a, c, u, or g
  misc_feature (47)..(317): /note=“This region may encompass 1-271 nucleotides”
  modified_base (328)..(329): a, c, u, or g
  modified_base (332)..(334): a, c, u, or g
  modified_base (336)..(339): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nvsnnbndurgybyrrnywggbhrdvrydbnnbnyunaurannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndsywydannchnnnhnnnnsba

SEQ ID NO: 590 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (326)..(327): a, c, u, or g
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsyybrkurgykyrruyggyhagmgcrygkurcudauaayryyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnryrrsywydanncycrymyngrsca

SEQ ID NO: 591 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsyysrkurgykcaruyggyhagmgcgyggurcudauaayrcyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngygrgyucrabhcycrymybgrsca

SEQ ID NO: 592 - RNA, 341 nt, Artificial Sequence
  source: /note=“Description of Artificial Sequence Synthetic polynucleotide”
  modified_base (1)..(1): a, c, u, or g
  modified_base (3)..(6): a, c, u, or g
  modified_base (11)..(12): a, c, u, or g
  modified_base (16)..(17): a, c, u, or g
  modified_base (21)..(22): a, c, u, or g
  modified_base (25)..(26): a, c, u, or g
  modified_base (28)..(31): a, c, u, or g
  modified_base (41)..(41): a, c, u, or g
  modified_base (43)..(316): a, c, u, or g
  misc_feature (46)..(316): /note=“This region may encompass 1-271 nucleotides”
  modified_base (318)..(319): a, c, u, or g
  modified_base (327)..(327): a, c, u, or g
  modified_base (331)..(332): a, c, u, or g
  modified_base (335)..(338): a, c, u, or g
  modified_base (340)..(340): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
nbnnnndursnnbarnnhggnnddnnbnnnnvdhycauahnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgdusdanhmynnbhnnnnvnw

SEQ ID NO: 593 - RNA, 339 nt, Artificial Sequence
  source: /note=“Description of Artificial Sequence Synthetic polynucleotide”
  modified_base (4)..(4): a, c, u, or g
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
rbcnbvkurgygcagydgghagyryryyrgkyycauaahyyvrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsdkrgdusdadmmymwsmbvbgsya

SEQ ID NO: 594 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
rschbvkurgygcagydgghagcryryyrgkyycauaaycyrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnswkrgwusdadmcymwsmbvkgsya

SEQ ID NO: 595 - RNA, 342 nt, Artificial Sequence
  source: /note=“Description of Artificial Sequence Synthetic polynucleotide”
  modified_base (2)..(7): a, c, u, or g
  modified_base (9)..(12): a, c, u, or g
  modified_base (16)..(16): a, c, u, or g
  modified_base (23)..(23): a, c, u, or g
  modified_base (26)..(26): a, c, u, or g
  modified_base (28)..(34): a, c, u, or g
  modified_base (37)..(37): a, c, u, or g
  modified_base (41)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (335)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
vnnnnnnunnnnvrrnhhgkhhndhnvnnnnnnnhynardnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvbyuhvanymbnnbnnnnnnbr

SEQ ID NO: 596 - RNA, 342 nt, Artificial Sequence
  source: /note=“Description of Artificial Sequence Synthetic polynucleotide”
  modified_base (6)..(6): a, c, u, or g
  modified_base (33)..(33): a, c, u, or g
  modified_base (41)..(41): a, c, u, or g
  modified_base (43)..(43): a, c, u, or g
  modified_base (46)..(317): a, c, u, or g
  misc_feature (47)..(317): /note=“This region may encompass 1-271 nucleotides”
  modified_base (319)..(319): a, c, u, or g
  modified_base (328)..(328): a, c, u, or g
  modified_base (333)..(333): a, c, u, or g
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbbvvndurkhhsrrbhygkhhurwbrhdbhdnryuhardnynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbyuhvanymybnbbnbbvnya

SEQ ID NO: 597 - RNA, 342 nt, Artificial Sequence
  source: /note=“Description of Artificial Sequence Synthetic polynucleotide”
  modified_base (6)..(6): a, c, u, or g
  modified_base (33)..(33): a, c, u, or g
  modified_base (43)..(43): a, c, u, or g
  modified_base (46)..(317): a, c, u, or g
  misc_feature (47)..(317): /note=“This region may encompass 1-271 nucleotides”
  modified_base (319)..(319): a, c, u, or g
  modified_base (333)..(333): a, c, u, or g
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations  for variant positions”
  source: /note=“See specification as filed for detailed description of substitutions and preferred embodiments”
rbbvvndurkhhsrrbhygkhhurwkrhdbhdnryuhardhynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbyuhvavymybnbynbbvnya

SEQ ID NO: 598 - RNA, 342 nt, Artificial Sequence
  source: /note=“Description of Artificial Sequence Synthetic polynucleotide”
  modified_base (1)..(7): a, c, u, or g
  modified_base (11)..(12): a, c, u, or g
  modified_base (16)..(16): a, c, u, or g
  modified_base (21)..(21): a, c, u, or g
  modified_base (26)..(26): a, c, u, or g
  modified_base (30)..(32): a, c, u, or g
  modified_base (44)..(320): a, c, u, or g
  misc_feature (47)..(317): /note=“This region may encompass 1-271 nucleotides”
  modified_base (328)..(328): a, c, u, or g
  modified_base (332)..(333): a, c, u, or g
  modified_base (335)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nnnnnnnursnnharnhwrrnuudrnrydnnnsvyyyuummbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvryuyrwnhybnnbnnnnnnhd

SEQ ID NO: 599 - RNA, 340 nt, Artificial Sequence
  source: /note=“Description of Artificial Sequence Synthetic polynucleotide”
  modified_base (1)..(1): a, c, u, or g
  modified_base (6)..(6): a, c, u, or g
  modified_base (11)..(11): a, c, u, or g
  modified_base (29)..(29): a, c, u, or g
  modified_base (43)..(43): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (333)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
nhvbdnvkrsnhharbhrrbudrdrydbndgryyyuummymhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgryuyrwbyysynbnnhndrhr

SEQ ID NO: 600 - RNA, 340 nt, Artificial Sequence
  source: /note=“Description of Artificial Sequence Synthetic polynucleotide”
  misc_feature (45)..(315): /note=“This region may encompass 1-271 nucleotides”
  modified_base (45)..(316): a, c, u, or g
  modified_base (333)..(333): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
vhvbrdvkascwharuhrrbudrwrcrkvdgacuyuumauchswnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrgryuyrwkyysyhbnhynkryr

SEQ ID NO: 601 - RNA, 341 nt, Artificial Sequence
  source: /note=“Description of Artificial Sequence Synthetic polynucleotide”
  modified_base (4)..(6): a, c, u, or g
  modified_base (29)..(31): a, c, u, or g
  modified_base (33)..(33): a, c, u, or g
  modified_base (41)..(41): a, c, u, or g
  modified_base (43)..(318): a, c, u, or g
  misc_feature (46)..(316): /note=“This region may encompass 1-271 nucleotides”
  modified_base (327)..(327): a, c, u, or g
  modified_base (331)..(332): a, c, u, or g
  modified_base (335)..(338): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
kbbnnnduagyyyarhyuggbwwgrryrnnnbnyygaarvnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdguucranychnnbhnnnnvma

SEQ ID NO: 602 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbyvwrruwgyyyaruuggsuagrrydykrbrcygaarrysyhmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyckgguucrayycysgguywbrvma

SEQ ID NO: 603 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gcyrarauwgcucaruugggagagyguuasacygaagaucuwmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnycugguucrayycyggguuucrvca

SEQ ID NO: 604 - RNA, 342 nt, Artificial Sequence
  source: /note=“Description of Artificial Sequence Synthetic polynucleotide”
  modified_base (4)..(6): a, c, u, or g
  modified_base (16)..(16): a, c, u, or g
  modified_base (21)..(21): a, c, u, or g
  modified_base (29)..(34): a, c, u, or g
  modified_base (37)..(37): a, c, u, or g
  modified_base (41)..(320): a, c, u, or g
  misc_feature (47)..(317): /note=“This region may encompass 1-271 nucleotides”
  modified_base (328)..(328): a, c, u, or g
  modified_base (331)..(334): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
sksnnnduvgbbyagnyygbnyyrdvvynnnnnnhunggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndguucranycnnnnhnnnbmsm

SEQ ID NO: 605 - RNA, 338 nt, Artificial Sequence
  source: /note=“Description of Artificial Sequence Synthetic polynucleotide”
  modified_base (39)..(39): a, c, u, or g
  modified_base (43)..(313): a, c, u, or g
  misc_feature (43)..(313): /note=“This region may encompass 1-271 nucleotides”
  variation (1)..(338): /replace=“ ”
  misc_feature (1)..(338): /note=“Variant nucleotides given in the sequence have no preference with respect to those in the annotations for variant positions”
  source: /note=“See specification as filed for detailed description of substitutions and preferred embodiments”
ggcusguuggkcuagkgburugruucucrsyuhggrysnragnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncyggguucaaaucvyrgasgagccc

SEQ ID NO: 606 - RNA, 338 nt, Artificial Sequence
  source: /note=“Description of Artificial Sequence Synthetic polynucleotide”
  modified_base (43)..(313): a, c, u, or g
  misc_feature (43)..(313): /note=“This region may encompass 1-271 nucleotides”
  variation (1)..(338): /replace=“ ”
  misc_feature (1)..(338): /note=“Variant nucleotides given in the sequence have no preference with respect to those in the annotations for variant positions”
  source: /note=“See specification as filed for detailed description of substitutions and preferred embodiments”
ggcucguuggucuagkggurugruucucgsuuhggrysbgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccggguucaaaucccggacgagccc

SEQ ID NO: 607 - RNA, 342 nt, Artificial Sequence
  source: /note=“Description of Artificial Sequence Synthetic polynucleotide”
  modified_base (2)..(7): a, c, u, or g
  modified_base (9)..(13): a, c, u, or g
  modified_base (16)..(16): a, c, u, or g
  modified_base (21)..(21): a, c, u, or g
  modified_base (25)..(28): a, c, u, or g
  modified_base (30)..(34): a, c, u, or g
  modified_base (37)..(37): a, c, u, or g
  modified_base (42)..(320): a, c, u, or g
  misc_feature (47)..(317): /note=“This region may encompass 1-271 nucleotides”
  modified_base (332)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
dnnnnnnynnnnnarnbhggnbbannnndnnnnnyynswrmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrkuuyradycynnnnnnnnnbd

SEQ ID NO: 608 - RNA, 341 nt, Artificial Sequence
  source: /note=“Description of Artificial Sequence Synthetic polynucleotide”
  modified_base (2)..(5): a, c, u, or g
  modified_base (36)..(36): a, c, u, or g
  modified_base (46)..(316): a, c, u, or g
  misc_feature (46)..(316): /note=“This region may encompass 1-271 nucleotides”
  modified_base (319)..(319): a, c, u, or g
  modified_base (331)..(331): a, c, u, or g
  modified_base (335)..(336): a, c, u, or g
  modified_base (338)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
knnnnbdurgchsarkbugguuavdghrdbkdrcynswraybmvdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvyngkuuyradyccnrbhnnbnnyd

SEQ ID NO: 609 - RNA, 341 nt, Artificial Sequence
  source: /note=“Description of Artificial Sequence Synthetic polynucleotide”
  modified_base (2)..(3): a, c, u, or g
  modified_base (5)..(5): a, c, u, or g
  modified_base (36)..(36): a, c, u, or g
  modified_base (46)..(316): a, c, u, or g
  misc_feature (46)..(316): /note=“This region may encompass 1-271 nucleotides”
  modified_base (335)..(336): a, c, u, or g
  modified_base (338)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
knndnbrurgchsarkcugguuavdghrwykrrcunswrayymrdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvydgguuyraaycchrbynnynnyk

SEQ ID NO: 610 - RNA, 341 nt, Artificial Sequence
  source: /note=“Description of Artificial Sequence Synthetic polynucleotide”
  modified_base (4)..(7): a, c, u, or g
  modified_base (12)..(12): a, c, u, or g
  modified_base (16)..(16): a, c, u, or g
  modified_base (25)..(25): a, c, u, or g
  modified_base (29)..(31): a, c, u, or g
  modified_base (33)..(33): a, c, u, or g
  modified_base (36)..(36): a, c, u, or g
  modified_base (41)..(41): a, c, u, or g
  modified_base (43)..(319): a, c, u, or g
  misc_feature (46)..(316): /note=“This region may encompass 1-271 nucleotides”
  modified_base (327)..(327): a, c, u, or g
  modified_base (331)..(337): a, c, u, or g
  modified_base (341)..(341): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
rbbnnnnurgynhadnhykghyrdnryrnnnsnhynguwansnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndgwyyvanbyhnnnnnnnrvyn

SEQ ID NO: 611 - RNA, 340 nt, Artificial Sequence
  source: /note=“Description of Artificial Sequence Synthetic polynucleotide”
  modified_base (4)..(4): a, c, u, or g
  modified_base (6)..(6): a, c, u, or g
  modified_base (12)..(12): a, c, u, or g
  modified_base (24)..(24): a, c, u, or g
  modified_base (45)..(315): a, c, u, or g
  misc_feature (45)..(315): /note=“This region may encompass 1-271 nucleotides”
  modified_base (317)..(317): a, c, u, or g
  modified_base (326)..(326): a, c, u, or g
  modified_base (331)..(332): a, c, u, or g
  modified_base (334)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
rbbnbnduvgynharbygghyrdnryryhbsbcyhguwavsvdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvndrgwuyvanbyyhnnbndnrvyb

SEQ ID NO: 612 - RNA, 340 nt, Artificial Sequence
  source: /note=“Description of Artificial Sequence Synthetic polynucleotide”
  modified_base (4)..(4): a, c, u, or g
  modified_base (6)..(6): a, c, u, or g
  modified_base (12)..(12): a, c, u, or g
  modified_base (24)..(24): a, c, u, or g
  modified_base (45)..(315): a, c, u, or g
  misc_feature (45)..(315): /note=“This region may encompass 1-271 nucleotides”
  modified_base (326)..(326): a, c, u, or g
  modified_base (332)..(332): a, c, u, or g
  modified_base (334)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
rsynbnduvgynharbugghyrdnryryhksbcyhguaavsmdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvhdrgwuyvanbyyhdnbndnrsyu

SEQ ID NO: 613 - RNA, 342 nt, Artificial Sequence
  source: /note=“Description of Artificial Sequence Synthetic polynucleotide”
  modified_base (4)..(6): a, c, u, or g
  modified_base (29)..(32): a, c, u, or g
  modified_base (34)..(34): a, c, u, or g
  modified_base (42)..(42): a, c, u, or g
  modified_base (44)..(317): a, c, u, or g
  misc_feature (47)..(317): /note=“This region may encompass 1-271 nucleotides”
  modified_base (319)..(319): a, c, u, or g
  modified_base (321)..(321): a, c, u, or g
  modified_base (328)..(328): a, c, u, or g
  modified_base (333)..(333): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rvvnnndurrybhhrhyhgghyuadvvynnnnrnbuccavanbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnrnguucranychynbhnnnbbhd

SEQ ID NO: 614 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
grmmkvrkggysmaryuggharssykkybrrsuccasakbvrwnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsygkguucradycmcrkyksbgyma

SEQ ID NO: 615 - RNA, 339 nt, Artificial Sequence; identical to SEQ ID NO: 614
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”

SEQ ID NO: 616 - RNA, 342 nt, Artificial Sequence
  source: /note=“Description of Artificial Sequence Synthetic polynucleotide”
  modified_base (3)..(6): a, c, u, or g
  modified_base (9)..(10): a, c, u, or g
  modified_base (12)..(13): a, c, u, or g
  modified_base (16)..(16): a, c, u, or g
  modified_base (25)..(26): a, c, u, or g
  modified_base (30)..(32): a, c, u, or g
  modified_base (42)..(42): a, c, u, or g
  modified_base (44)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (336)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
bbnnnndunnynnarnyugsyhwrnnrbdnnnrdcuruarvnynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvgwuyranucbnnbhnnnnnvd

SEQ ID NO: 617 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
bbkkbdauagyucagbugsyuagagydkwkracuruagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnscuggwuyrahucyrrsubsnmsvd

SEQ ID NO: 618 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
bbkksdauagyucagyuggyuagagcdkwkgacuruagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnscugguuyrawuccrrsuysdmsva

SEQ ID NO: 619 - RNA, 341 nt, Artificial Sequence
  source: /note=“Description of Artificial Sequence Synthetic polynucleotide”
  modified_base (2)..(2): a, c, u, or g
  modified_base (4)..(7): a, c, u, or g
  modified_base (12)..(12): a, c, u, or g
  modified_base (15)..(16): a, c, u, or g
  modified_base (21)..(21): a, c, u, or g
  modified_base (24)..(25): a, c, u, or g
  modified_base (28)..(28): a, c, u, or g
  modified_base (30)..(33): a, c, u, or g
  modified_base (36)..(36): a, c, u, or g
  modified_base (40)..(319): a, c, u, or g
  misc_feature (46)..(316): /note=“This region may encompass 1-271 nucleotides”
  modified_base (327)..(327): a, c, u, or g
  modified_base (330)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dndnnnnuvrynbrnnhugknyannrynbnnnnhunacrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrdubranhynnnnnnnnnnyv

SEQ ID NO: 620 - RNA, 340 nt, Artificial Sequence
  source: /note=“Description of Artificial Sequence Synthetic polynucleotide”
  modified_base (15)..(16): a, c, u, or g
  modified_base (24)..(24): a, c, u, or g
  modified_base (27)..(27): a, c, u, or g
  modified_base (29)..(30): a, c, u, or g
  modified_base (41)..(43): a, c, u, or g
  modified_base (45)..(315): a, c, u, or g
  misc_feature (45)..(315): /note=“This region may encompass 1-271 nucleotides”
  modified_base (317)..(318): a, c, u, or g
  modified_base (326)..(326): a, c, u, or g
  modified_base (330)..(330): a, c, u, or g
  modified_base (333)..(333): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbkbshdurrydbrnnhgkuyadnrynynndhyuhacrhknnnrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnrrdubranhybndvndsdwvyv

SEQ ID NO: 621 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (317)..(318): a, c, u, or g
  modified_base (330)..(330): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbkbsydurgydbrbhugkuyadhryvyhhdyyuhacahkhddrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnngrdubradhyyndvhrsdwvya

SEQ ID NO: 622 - RNA, 72 nt, Artificial Sequence
  source: /note=“Description of Artificial Sequence Synthetic oligonucleotide”
ggcuccguggcgcaauggauagcgcauuggacuucuaauucaaagguuccggguucggucccggcggagucg

SEQ ID NO: 623 - RNA, 72 nt, Artificial Sequence
  source: /note=“Description of Artificial Sequence Synthetic oligonucleotide”
  modified_base (27)..(27): Pseudouridine
ggcuccguggcgcaauggauagcgcanuggacuucuaauucaaagguuccggguucggucccggcggagucg

SEQ ID NO: 624 - DNA, 75 bp, Artificial Sequence
  source: /note=“Description of Artificial Sequence Synthetic oligonucleotide”
  source: /note=“Description of Combined DNA/RNA Molecule Synthetic oligonucleotide”
  modified_base (19)..(19): Dihydrouridine
gguuccaugguguaauggnaagcacucuggacuctgaauccagcgauccgaguucgagucucgguggaaccucca

SEQ ID NO: 625 - DNA, 75 bp, Artificial Sequence
  source: /note=“Description of Artificial Sequence Synthetic oligonucleotide”
  source: /note=“Description of Combined DNA/RNA Molecule Synthetic oligonucleotide”
  modified_base (13)..(13): Pseudouridine
gguuccauggugnaaugguaagcacucuggacuctgaauccagcgauccgaguucgagucucgguggaaccucca

SEQ ID NO: 626 - RNA, 73 nt, Artificial Sequence
  source: /note=“Description of Artificial Sequence Synthetic oligonucleotide”
gcccggcuagcucagucgguagagcaugggacucuuaaucccagggucguggguucgagccccacguugggcg

SEQ ID NO: 627 - DNA, 72 bp, Artificial Sequence; identical to SEQ ID NO: 262
  source: /note=“Description of Artificial Sequence Synthetic oligonucleotide”

SEQ ID NO: 628 - DNA, 20 bp, Artificial Sequence
  source: /note=“Description of Artificial Sequence Synthetic primer”
tgagttggcaacctgtggta

SEQ ID NO: 629 - DNA, 20 bp, Artificial Sequence
  source: /note=“Description of Artificial Sequence Synthetic primer”
ttgggtgtccatgaaaatca

SEQ ID NO: 630 - RNA, 26 nt, Artificial Sequence
  source: /note=“Description of Artificial Sequence Synthetic probe”
uagcagaggaugguuucgauccauca

SEQ ID NO: 631 - DNA, 26 bp, Artificial Sequence
  source: /note=“Description of Artificial Sequence Synthetic probe”
tagcagaggatggtttcgatccatca

SEQ ID NO: 632 - DNA, 82 bp, Artificial Sequence; identical to SEQ ID NO: 192
  source: /note=“Description of Artificial Sequence Synthetic oligonucleotide”

SEQ ID NO: 633 - DNA, 49 bp, Artificial Sequence
  source: /note=“Description of Artificial Sequence Synthetic probe”
aaaacatcagcaccggctcaccaattccgctacctatctttaggtaacc

SEQ ID NO: 634 - DNA, 73 bp, Artificial Sequence; identical to SEQ ID NO: 166
  source: /note=“Description of Artificial Sequence Synthetic oligonucleotide”

SEQ ID NO: 635 - RNA, 15 nt, Artificial Sequence
  source: /note=“Description of Artificial Sequence Synthetic probe”
cagauuaaaagucug

SEQ ID NO: 636 - RNA, 77 nt, Artificial Sequence
  source: /note=“Description of Artificial Sequence Synthetic oligonucleotide”
ggcucuauggcuuaguugguuaaagcgccugucucguaaacaggagauccuggguucgacucccaguggggccucaa

SEQ ID NO: 637 - RNA, 84 nt, Artificial Sequence
  source: /note=“Description of Artificial Sequence Synthetic oligonucleotide”
gucaggauggccgaguggucuaaggcgccagacucaaguucuggucuccguauggaggcguggguucgaaucccacuucugaca

SEQ ID NO: 638 - DNA, 71 bp, Artificial Sequence; identical to SEQ ID NO: 108
  source: /note=“Description of Artificial Sequence Synthetic oligonucleotide”

SEQ ID NO: 639 - DNA, 20 bp, Artificial Sequence
  source: /note=“Description of Artificial Sequence Synthetic oligonucleotide”
cagtgtgtgaagacggctgc

SEQ ID NO: 640 - RNA, 72 nt, Artificial Sequence
  source: /note=“Description of Artificial Sequence Synthetic oligonucleotide”
ggcucguuggucuagggguaugauucucgcuuagggugcgagaggucccggguucaaaucccggacgagccc

SEQ ID NO: 641 - RNA, 72 nt, Artificial Sequence
  source: /note=“Description of Artificial Sequence Synthetic oligonucleotide”
ggggauguagcucagugguagagcgcaugcuuugcauguaugaggucccggguucgauccccggcaucucca

SEQ ID NO: 642 - RNA, 71 nt, Artificial Sequence
  source: /note=“Description of Artificial Sequence Synthetic oligonucleotide”
ggcucguuggucuagggguaugaucucgcuuagggugcgagaggucccggguucaaaucccggacgagccc

SEQ ID NO: 643 - RNA, 74 nt, Artificial Sequence
  source: /note=“Description of Artificial Sequence Synthetic oligonucleotide”
ggcucuguggcuuaguuggcuaaagcgccugucucguaaacaggagauccuggguucgaaucccagcggggccu

SEQ ID NO: 644 - protein, 8 aa, Artificial Sequence
  source: /note=“Description of Artificial Sequence Synthetic peptide”
DYKDDDDK

SEQ ID NO: 645 - RNA, 82 nt, Artificial Sequence
  source: /note=“Description of Artificial Sequence Synthetic oligonucleotide”
gguagcguggccgagcggucuaaggcgcuggauuaaggcuccagucucuucggaggcguggguucgaaucccaccgcugcca

SEQ ID NO: 646 - RNA, 74 nt, Artificial Sequence
  source: /note=“Description of Artificial Sequence Synthetic oligonucleotide”
ggcgccguggcuuaguugguuaaagcgccugucuaguaaacaggagauccuggguucgaaucccagcggugccu

(19) United States
(12) Patent Application Publication
(10) Pub. No.: US 2023/0203509 A1
(43) Pub. Date: Jun. 29, 2023

(54) TREM COMPOSITIONS AND METHODS RELATING THERETO

(71) Applicant: Flagship Pioneering Innovations VI, LLC, Cambridge, MA (US)

(72) Inventors: Theonie Anastassiadis, Boston, MA (US); David Arthur Berry, Newton, MA (US); Christine Elizabeth Hajdin, Boston, MA (US); Noubar Boghos Afeyan, Lexington, MA (US); David Charles Donnell Butler, Medford, MA (US); Qingyi Li, Somerville, MA (US)

(21) Appl. No.: 17/928,450

(22) PCT Filed: May 28, 2021
(86) PCT No.: PCT/US2021/035015

§ 371 Date: Nov. 29, 2022

Related U.S. Application Data
(60) Provisional application No. 63/032,008, filed on May 29, 2020.

(51) Int. Cl. C12N 15/67; C12N 15/11

(52) U.S. Cl. CPC C12N 15/67; C12N 15/11; C12N 2310/10; C12N 2320/34

(57) ABSTRACT

The disclosure relates generally to uses of tRNA-based effector molecules having a non-naturally occurring modification.

### CROSS-REFERENCE TO RELATED APPLICATIONS

This application claims priority to U.S. Provisional Application No. 63/032,008, filed on May 29, 2020, the entire contents of which is hereby incorporated by reference.

### BACKGROUND

Transfer RNAs (tRNAs) are complex, naturally occurring RNA molecules that possess a number of functions including initiation and elongation of proteins.

### SUMMARY

The present disclosure features modified tRNA-based effector molecules (TREMs, e.g., a TREM, TREM core fragment, or TREM fragment), as well as related compositions and uses thereof. The inventors have discovered that a TREM composition can be used, inter alia, to: (1) modulate a production parameter of an RNA, or a protein encoded by an RNA, wherein the RNA has a contextually-rare codon (“con-rare codon”); and/or (2) to modulate tRNA pools in a cell or a subject. Accordingly, in an aspect, provided herein is a method of modulating a tRNA pool in a cell comprising an endogenous open reading frame (ORF), which ORF comprises a codon having a first sequence, comprising: optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell; contacting the cell with a TREM composition comprising a TREM, a TREM core fragment, or a TREM fragment described herein, wherein the TREM, TREM core fragment or TREM fragment has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the cell, thereby modulating the tRNA pool in the cell.

In an embodiment, the TREM comprises an anticodon that pairs with (a). In an embodiment, the TREM comprises an anticodon that pairs with (b).

In another aspect, provided herein is a method of modulating a tRNA pool in a subject having an endogenous open reading frame (ORF), which ORF comprises a codon having a first sequence, comprising: optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject; contacting the subject with a TREM composition comprising a TREM, a TREM core fragment, or a TREM fragment described herein, wherein the TREM, TREM core fragment or TREM fragment has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the subject, thereby modulating the tRNA pool in the subject.

In an embodiment, the TREM comprises an anticodon that pairs with (a). In an embodiment, the TREM comprises an anticodon that pairs with (b).

In an embodiment, the method comprises acquiring knowledge of (i). In an embodiment, the method comprises acquiring knowledge of (ii). In an embodiment, the method comprises acquiring knowledge of (i) and (ii).

In an embodiment, acquiring knowledge of (i) comprises acquiring a value for the abundance, e.g., relative amounts, of (i); and/or acquiring knowledge of (ii) comprises acquiring a value for the abundance, e.g., relative amounts, of (ii). In an embodiment, responsive to said value, the cell or subject is contacted with the TREM composition having an anticodon that pairs with (a) or (b).

In one aspect, the disclosure provides, a method of evaluating a tRNA pool in a cell or subject, comprising an endogenous open reading frame (ORF), which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the cell or subject wherein:

(i) is a tRNA moiety having an anticodon that pairs with the codon of the ORF having a first sequence (the first tRNA moiety); and

(ii) is an isoacceptor tRNA moiety having an anticodon that pairs with a codon other than the codon having the first sequence (the second tRNA moiety) in the cell or subject,

thereby evaluating the tRNA pool in the cell or subject.

In an embodiment, the method comprises acquiring knowledge of (i). In an embodiment, the method comprises acquiring knowledge of (ii). In an embodiment, the method comprises acquiring knowledge of (i) and (ii).

In an embodiment, acquiring knowledge of (i) comprises acquiring a value for the abundance, e.g., relative amount, of (i); and/or acquiring knowledge of (ii) comprises acquiring a value for the abundance, e.g., relative amount, of (ii). In an embodiment, responsive to said value, the method comprises administering a TREM composition comprising a TREM, a TREM core fragment, or a TREM fragment described herein, wherein the TREM, TREM core fragment or TREM fragment has an anticodon that pairs with: (a) the codon having the first sequence; or (b) the codon other than the codon having the first sequence, in an amount and for a time sufficient to modulate the relative amounts of the first tRNA moiety and the second tRNA moiety.

In yet another aspect, provided herein is a method of modulating a tRNA pool in a subject or cell having an endogenous open reading frame (ORF) comprising a codon comprising a synonymous mutation (a synonymous mutation codon or SMC), comprising:

providing a TREM composition comprising a TREM, a TREM core fragment, or a TREM fragment described herein, wherein the TREM, TREM core fragment or TREM fragment comprises an isoacceptor tRNA moiety comprising an anticodon sequence that pairs with the SMC (the TREM);

contacting the subject or cell with the TREM composition in an amount and/or for a time sufficient to modulate the tRNA pool in the subject or cell,

thereby modulating the tRNA pool in the subject or cell.

In an embodiment, the method comprises acquiring knowledge of the abundance of one or both of (i) and (ii) e.g., acquiring knowledge of the relative amounts of (i) and (ii) wherein (i) is a tRNA moiety having an anticodon that pairs with the SMC (the first tRNA moiety) and (ii) is an isoacceptor tRNA moiety having an anticodon that pairs with a codon other than the SMC (the second tRNA moiety), in the subject or cell.

In one aspect, the disclosure provides a method of treating a subject having an endogenous open reading frame (ORF) comprising a codon having a first sequence, comprising:

(i) acquiring, e.g., directly or indirectly acquiring, a value for the status of the codon having the first sequence in the subject, wherein said value comprises a measure of the presence or absence of the codon having the first sequence in a sample from the subject; and identifying the subject as having the codon having the first sequence; and

(ii) responsive to said value, administering a TREM composition comprising a TREM, a TREM core fragment, or a TREM fragment described herein, wherein the TREM, TREM core fragment or TREM fragment comprises an isoacceptor tRNA moiety having an anticodon that pairs with the codon having the first sequence, to the subject,

thereby treating the subject.

In yet another aspect, provided herein is a method of treating a subject having an endogenous open reading frame (ORF) comprising a codon comprising a synonymous mutation (a synonymous mutation codon or SMC), comprising:

(i) acquiring, e.g., directly or indirectly acquiring, a value for the SMC status of the subject, wherein said value comprises a measure of the presence or absence of SMC in a sample from the subject, and identifying the subject as having a SMC; and

(ii) responsive to said value, administering a TREM composition comprising a TREM, a TREM core fragment, or a TREM fragment described herein, wherein the TREM, TREM core fragment or TREM fragment comprises comprising an isoacceptor tRNA moiety having an anticodon that pairs with the SMC, to the subject,

thereby treating the subject.

In an aspect, the disclosure provides a method of evaluating a subject having an endogenous open reading frame (ORF) comprising a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

identifying the subject as having a codon having the first sequence,

thereby evaluating the subject.

In another aspect, provided herein is a method of evaluating a subject having an endogenous open reading frame (ORF) comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject; and

identifying the subject as having a SMC,

thereby evaluating the subject.

In an embodiment of any of the methods disclosed herein, the TREM, TREM core fragment or TREM fragment does not comprise an anticodon that pairs with a stop codon.

In an embodiment of any of the methods disclosed herein, (a) the ORF codon having the first sequence; or (b) the SMC; is other than a stop codon, e.g., TAA, TGA or TAG.

In an embodiment, (a) the ORF codon having the first sequence; or (b) the SMC; in the absence of contact with the composition comprising a TREM, is associated with a phenotype, e.g., an unwanted phenotype, e.g., a disorder or symptom, e.g., a disorder or symptom chosen from Table 1. In an embodiment, the disorder or symptom is chosen from a disease group provided in Table 1, e.g., cardiovascular, dermatology, endocrine, immunology, neurology, oncology, ophthalmology, or respiratory.

In one aspect, disclosed herein is a method of modulating a production parameter of an RNA, or a protein encoded by an RNA, in a target cell or tissue, comprising:

providing, e.g., administering, to the target cell or tissue, or contacting the target cell or tissue with, an effective amount of a TREM composition comprising a TREM, a TREM core fragment, or a TREM fragment described herein, which TREM, TREM core fragment or TREM fragment corresponds to a contextually-rare codon (“con-rare codon”) of the RNA,

thereby modulating the production parameter of the RNA, or protein encoded by the RNA in the target cell or tissue.

In an embodiment, the target cell or tissue is obtained from a subject.

In an embodiment, the method comprises administering the TREM composition to a subject.

In an embodiment, the method comprises contacting the TREM composition with the target tissue or cell ex vivo.

In an embodiment, the method comprises introducing the ex vivo-contacted target tissue or cell into a subject, e.g., an allogeneic or autologous subject.

In an embodiment, the target cell or tissue is a specific or selected target cell or tissue, e.g., a cell or tissue type in a particular developmental stage; a cell or tissue type in a particular disease state; or a cell present in a particular extracellular milieu.

In an embodiment of any of the methods disclosed herein, the production parameter comprises an expression parameter or a signaling parameter, e.g., as described herein. In an embodiment, the production parameter of the RNA is modulated, e.g., an RNA that can be translated into a polypeptide, e.g., a messenger RNA. In an embodiment, the production parameter of the RNA is increased or decreased. In an embodiment, the production parameter of the protein encoded by the RNA is modulated. In an embodiment, the production parameter of the protein is increased. In an embodiment, the production parameter of the protein is decreased.

In another aspect, provided herein is a method of determining the presence of a nucleic acid sequence, e.g., a DNA or RNA, having a contextually-rare codon (“con-rare codon nucleic acid sequence”), comprising:

acquiring knowledge of the presence of the con-rare codon nucleic acid sequence in a sample from a subject, e.g., a target cell or tissue sample,

wherein responsive to the acquisition of knowledge of the presence of the con-rare codon nucleic acid sequence:

(1) the subject is classified as being a candidate to receive administration of an effective amount of a TREM composition comprising a TREM, a TREM core fragment, or a TREM fragment described herein, which TREM, TREM core fragment or TREM fragment corresponds to a contextually-rare codon (“con-rare codon”) of the nucleic acid sequence; or

(2) the subject is identified as likely to respond to a treatment comprising the TREM composition.

In yet another aspect, the disclosure provides a method of treating a subject having a disease associated with a contextually-rare codon (“con-rare codon”), comprising:

acquiring knowledge of the presence of a nucleic acid sequence, e.g., a DNA or RNA, having the con-rare codon (“con-rare codon nucleic acid sequence”) in a target cell or tissue sample from the subject; and

administering to the subject an effective amount of a TREM composition comprising a TREM, a TREM core fragment, or a TREM fragment described herein, which TREM, TREM core fragment or TREM fragment corresponds to the con-rare codon of the nucleic acid sequence,

thereby treating the disease in the subject.

In an aspect, provided herein is a method of providing a tRNA effector molecule (TREM) to a subject, comprising:

providing, e.g., administering, to the subject, an effective amount of a TREM composition comprising a TREM, a TREM core fragment, or a TREM fragment described herein, which TREM, TREM core fragment or TREM fragment corresponds to a contextually-rare codon (“con-rare codon”) for a nucleic acid sequence in a target cell or tissue in the subject,

thereby providing a TREM composition to the subject.

In an embodiment of any of the methods disclosed herein, the method comprises acquiring a value for a con-rare codon in the nucleic acid sequence, e.g., DNA or RNA, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon;

In an embodiment, (1) comprises determining the presence or absence of a con-rare codon.

In an embodiment, a determination of the availability of a tRNA comprises acquiring a measure of one, two, three or all of the following parameters:

(a) level of a tRNA corresponding to the con-rare codon (“con-rare codon tRNA”) compared to a tRNA corresponding to a different codon;

(b) function, e.g., polypeptide chain elongation function, of a con-rare codon tRNA compared to a tRNA corresponding to a different codon;

(c) modification, e.g., aminoacylation or post-transcriptional modification, of a con-rare codon tRNA compared to a tRNA corresponding to a different codon; and/or

(d) sequence of a con-rare codon tRNA.

In an embodiment of any of the methods disclosed herein, the method comprises acquiring a value of the knowledge of the abundance of e.g., acquiring knowledge of the relative amounts of: (i) a tRNA moiety having an anticodon that pairs with the con-rare codon (the first tRNA moiety).

In an embodiment of any of the methods disclosed herein, the method comprises acquiring a value of the knowledge of the abundance of e.g., acquiring knowledge of the relative amounts of: (ii) an isoacceptor tRNA moiety having an anticodon that pairs with a codon other than the con-rare codon (the second tRNA moiety).

In an embodiment, the method comprises acquiring a value of the knowledge (i) and (ii).

In an embodiment of any of the methods disclosed herein, at least 10%, 20%, 30%, 40%, 50%, 60%, 70%, 80%, 85%, 90%, 95%, 99%, or 100% (by weight or number) of the TREMs, TREM core fragments or TREM fragments in the TREM composition correspond to a con-rare codon.

In an embodiment of any of the methods disclosed herein, the TREM composition comprises TREMs, TREM core fragments or TREM fragments that correspond to a plurality of con-rare codons.

In an embodiment of any of the methods disclosed herein, the TREM composition comprises: a first TREM which corresponds to a first con-rare codon; and an additional TREM which corresponds to a different con-rare codon.

In an embodiment of any of the methods disclosed herein, the TREM composition comprises: a first TREM which corresponds to a first con-rare codon; and a second TREM which corresponds to a second con-rare codon.

In an embodiment of any of the methods disclosed herein, at least 10%, 20%, 30%, 40%, 50%, 60%, 70%, 80%, 85%, 90%, 95%, 99%, or 100% (by weight or number) of the TREMs in the composition are charged.

In an embodiment of any of the methods disclosed herein, at least 10%, 20%, 30%, 40%, 50%, 60%, 70%, 80%, 85%, 90%, 95%, 99%, or 100% (by weight or number) of the TREMs in the composition are of the same iso-decoder isotype.

In an embodiment of any of the methods disclosed herein, the cell is a host cell. In an embodiment, the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.

In an embodiment of any of the methods disclosed herein, the cell comprises an exogenous nucleic acid sequence. In an embodiment, the cell is autologous to the exogenous nucleic acid sequence. In an embodiment, the cell is allogeneic to the exogenous nucleic acid sequence. In an embodiment, the exogenous nucleic acid sequence (e.g., DNA or RNA) comprises a con-rare codon.

In an embodiment, administration of a TREM composition corresponding to the con-rare codon to the cell, modulates a production parameter, e.g., expression parameter or signaling parameter, of a product, e.g., RNA or polypeptide, of the exogenous nucleic sequence.

In an aspect, provided herein is a TREM comprising a sequence of Formula A:

[L1]-[ASt Domain1]-[L2]-[DH Domain]-[L3]-[ACH Domain]-[VL Domain]-[TH Domain]-[L4]-[ASt Domain2],

wherein independently, [L1] and [VL Domain], are optional; and one of [L1], [ASt Domain1], [L2]-[DH Domain], [L3], [ACH Domain], [VL Domain], [TH Domain], [L4], and [ASt Domain2] comprises a nucleotide having a non-naturally occurring modification.

In an embodiment, the TREM: (a) retains the ability to: support protein synthesis, be charged by a synthetase, be bound by an elongation factor, introduce an amino acid into a peptide chain, support elongation, or support initiation; (b) comprises at least X contiguous nucleotides without a non-naturally occurring modification, wherein X is greater than 10; (c) comprises at least 3, but less than all of the nucleotides of a type (e.g., A, T, C, G or U) comprise the same non-naturally occurring modification; (d) comprises at least X nucleotides of a type (e.g., A, T, C, G or U) that do not comprise a non-naturally occurring modification, wherein X=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50; (e) comprises no more than 5, 10, or 15 nucleotides of a type (e.g., A, T, C, G or U) that comprise a non-naturally occurring modification; and/or (f) comprises no more than 5, 10, or 15 nucleotides of a type (e.g., A, T, C, G or U) that do not comprise a non-naturally occurring modification.

In an embodiment, the TREM comprises feature (a). In an embodiment, the TREM comprises feature (b). In an embodiment, the TREM comprises feature (c). In an embodiment, the TREM comprises feature (d). In an embodiment, the TREM comprises feature (e). In an embodiment, the TREM comprises feature (f). In an embodiment, the TREM comprises all of features (a)-(f) or a combination thereof.

In an embodiment, the TREM Domain comprising the non-naturally occurring modification retains a function, e.g., a domain function described herein.

In an aspect, provided herein is a TREM core fragment comprising a sequence of Formula B:

[L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ,

wherein x=1 and y=0 or 1; and one of [ASt Domain1], [ACH Domain], and [ASt Domain2] comprises a nucleotide having a non-naturally occurring modification.

In an embodiment, the TREM retains the ability to support protein synthesis. In an embodiment, the TREM retains the ability to be able to be charged by a synthetase. In an embodiment, the TREM retains the ability to be bound by an elongation factor. In an embodiment, the TREM retains the ability to introduce an amino acid into a peptide chain. In an embodiment, the TREM retains the ability to support elongation. In an embodiment, the TREM retains the ability to support initiation.

In an embodiment, the [ASt Domain 1] and/or [ASt Domain 2] comprising the non-naturally occurring modification retains the ability to initiate or elongate a polypeptide chain.

In an embodiment, the [ACH Domain] comprising the non-naturally occurring modification retains the ability to mediate pairing with a codon.

In an embodiment, y=1 for any one, two, three, four, five, six, all or a combination of [L1], [L2], [DH Domain], [L3], [VL Domain], [TH Domain], [L4].

In an embodiment, y=0 for any one, two, three, four, five, six, all or a combination of [L1], [L2], [DH Domain], [L3], [VL Domain], [TH Domain], [L4].

In an embodiment, y=1 for linker [L1], and L1 comprises a nucleotide having a non-naturally occurring modification.

In an embodiment, y=1 for linker [L2], and L2 comprises a nucleotide having a non-naturally occurring modification.

In an embodiment, y=1 for [DH Domain (DHD)], and DHD comprises a nucleotide having a non-naturally occurring modification. In an embodiment, the DHD comprising the non-naturally occurring modification retains the ability to mediate recognition of aminoacyl-tRNA synthetase.

In an embodiment, y=1 for linker [L3], and L3 comprises a nucleotide having a non-naturally occurring modification.

In an embodiment, y=1 for [VL Domain (VLD)], and VLD comprises a nucleotide having a non-naturally occurring modification.

In an embodiment, y=1 for [TH Domain (THD)], and THD comprises a nucleotide having a non-naturally occurring modification. In an embodiment, the THD comprising the non-naturally occurring modification retains the ability to mediate recognition of the ribosome.

In an embodiment, y=1 for linker [L4], and L4 comprises a nucleotide having a non-naturally occurring modification.

In another aspect, the disclosure provides a TREM fragment comprising a portion of a TREM, wherein the TREM comprises a sequence of Formula A:

[L1]-[ASt Domain1]-[L2]-[DH Domain]-[L3]-[ACH Domain]-[VL Domain]-[TH Domain]-[L4]-[ASt Domain2], and wherein the TREM fragment comprises a non-naturally occurring modification.

In an embodiment, the TREM fragment comprises one, two, three or all or any combination of the following: (a) a TREM half (e.g., from a cleavage in the ACH Domain, e.g., in the anticodon sequence, e.g., a 5′ half or a 3′ half); (b) a 5′ fragment (e.g., a fragment comprising the 5′ end, e.g., from a cleavage in a DH Domain or the ACH Domain); (c) a 3′ fragment (e.g., a fragment comprising the 3′ end, e.g., from a cleavage in the TH Domain); or (d) an internal fragment (e.g., from a cleavage in any one of the ACH Domain, DH Domain or TH Domain).

In an embodiment, the TREM fragment comprise (a) a TREM half which comprises a nucleotide having a non-naturally occurring modification.

In an embodiment, the TREM fragment comprise (b) a 5′ fragment which comprises a nucleotide having a non-naturally occurring modification.

In an embodiment, the TREM fragment comprise (c) a 3′ fragment which comprises a nucleotide having a non-naturally occurring modification.

In an embodiment, the TREM fragment comprise (d) an internal fragment which comprises a nucleotide having a non-naturally occurring modification.

In an embodiment of any of the TREMs, TREM core fragments, or TREM fragments disclosed herein, the TREM Domain comprises a plurality of nucleotides each having a non-naturally occurring modification.

In an embodiment of any of the TREMs, TREM core fragments, or TREM fragments disclosed herein, the non-naturally occurring modification is a modification in a base or a backbone of a nucleotide, e.g., a modification chosen from any one of Tables 5, 6, 7, 8 or 9.

In an embodiment of any of the TREMs, TREM core fragments, or TREM fragments disclosed herein, the non-naturally occurring modification is a base modification chosen from a modification listed in Table 5.

In an embodiment of any of the TREMs, TREM core fragments, or TREM fragments disclosed herein, the non-naturally occurring modification is a base modification chosen from a modification listed in Table 6.

In an embodiment of any of the TREMs, TREM core fragments, or TREM fragments disclosed herein, the non-naturally occurring modification is a base modification chosen from a modification listed in Table 7.

In an embodiment of any of the TREMs, TREM core fragments, or TREM fragments disclosed herein, the non-naturally occurring modification is a backbone modification chosen from a modification listed in Table 8.

In an embodiment of any of the TREMs, TREM core fragments, or TREM fragments disclosed herein, the non-naturally occurring modification is a backbone modification chosen from a modification listed in Table 9.

In an embodiment of any of the TREMs, TREM core fragments, or TREM fragments disclosed herein, the TREM, TREM core fragment, or TREM fragment is encoded by a sequence provided in Table 4, e.g., any one of SEQ ID NOs 1-451.

In an embodiment of any of the TREMs, TREM core fragments, or TREM fragments disclosed herein, the TREM, TREM core fragment, or TREM fragment is encoded by a consensus sequence chosen from any one of SEQ ID NOs: 562-621.

In another aspect, the disclosure provides a pharmaceutical composition comprising a TREM, a TREM core fragment, or a TREM fragment disclosed herein.

In another aspect, the disclosure provides a method of making a TREM, a TREM core fragment, or a TREM fragment disclosed herein, comprising linking a first nucleotide to a second nucleotide to form the TREM.

In an embodiment, the TREM, TREM core fragment or TREM fragment is synthetic.

In an embodiment, the TREM, TREM core fragment or TREM fragment is made by cell-free solid phase synthesis.

In an embodiment of any of the TREMs, TREM core fragments, or TREM fragments disclosed herein, the TREM Domain comprises a plurality of nucleotides each having a non-naturally occurring modification. In an embodiment, the non-naturally occurring modification comprises a nucleobase modification, a sugar (e.g., ribose) modification, or a backbone modification. In an embodiment, tbe non-naturally occurring modification is a sugar (e.g., ribose) modification. In an embodiment, tbe non-naturally occurring modification is 2′-ribose modification, e.g., a 2′-OMe, 2′-halo (e.g., 2′-F), 2′-MOE, or 2′-deoxy modification. In an embodiment, tbe non-naturally occurring modification is a backbone modification, e.g., a phosphorothioate modification.

Additional features of any of the aforesaid TREMs, TREM core fragments, TREM fragments, TREM compositions, preparations, methods of making TREM compositions and preparations, and methods of using TREM compositions and preparations include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

Other features, objects, and advantages of the invention will be apparent from the Examples, Description, Figures, and Claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTIONS OF THE DRAWINGS

FIG. 1A depicts the mRNA and protein sequence, and the endogenous tRNA pool for a non-SNP subject. The sequence of the second codon is GTG (depicted by the open triangle) encoding for the amino acid valine. Two Valine isoacceptor tRNA species are shown. Each of the two tRNA species recognize different Valine codons. The two species have different abundances. The species that recognize the wildtype codon, GTG, are not shaded and are in higher abundance. The shaded species, which has a lower abundance, does not pair with the wildtype codon. Thus, the Valine isoacceptor tRNA species that corresponds to the codon used (GTG) is abundant.

FIG. 1B depicts the mRNA and protein sequence, and the endogenous tRNA pool for a subject having a single nucleotide polymorphism (SNP) at the third position of the second codon (shown with a closed triangle) in the depicted mRNA sequence. The composition of the endogenous tRNA pool is the same as described for FIG. 1A. However, incorporation of Valine at the second codon now depends on the use of a less abundant tRNA species (the shaded species). As a consequence, as shown in FIG. 1B, translation is compromised. Other consequences of using a less abundant tRNA species may also be, e.g., interruption of the elongation of the peptide chain, lower protein production, protein misfolding, protein mislocalization, altered protein function, or altered mRNA transcript stability.

FIG. 1C depicts the same mRNA sequence as in FIG. 1B, which includes a SNP at the third position of the second codon. The endogenous tRNAs of the pool are the same as in FIGS. 1A and 1B, but the pool is supplemented with exogenous TREMs which increase the abundance of species that will pair with the SNP codon. This can result in an improvement in the translation of the mRNA.

FIG. 2A depicts the mRNA and protein sequence, and the endogenous tRNA pool for a non-SNP subject. The sequence of the second codon is GTG (depicted by the open triangle) encoding for the amino acid Valine. Two Valine isoacceptor tRNA species are shown. Each of the two tRNA species recognize different Valine codons. The two species have different abundances. The species that recognize the wildtype codon, GTG, are not shaded and are in higher abundance. The shaded species, which has a lower abundance, does not pair with the wildtype codon. Thus, the Valine isoacceptor tRNA species that corresponds to the codon used (GTG) is abundant. This results in translation of the mRNA sequence into the corresponding protein as depicted.

FIG. 2B depicts the mRNA and protein sequence, and the endogenous tRNA pool for a subject having a single nucleotide polymorphism (SNP) at the third position of the second codon (shown with a closed triangle) in the depicted mRNA sequence. The composition of the endogenous tRNA pool is the same as described for FIG. 2A. However, incorporation of Valine at the second codon now depends on the use of a less abundant tRNA species (the shaded species). As a consequence, as shown in FIG. 2B, translation of the mRNA sequence into the corresponding protein is compromised.

FIG. 2C depicts the same mRNA sequence as in FIG. 2B, which includes a SNP at the third position of the second codon (shown with a closed triangle). The endogenous tRNAs of the pool are the same as in FIGS. 2A and 2B, but the pool is supplemented with exogenous TREMs which increase the abundance of species that will pair with the SNP codon. As a consequence, translation of the mRNA sequence into the corresponding protein is not compromised and is similar to that of a non-SNP subject.

FIG. 3 The top row depicts the endogenous tRNA pool and, moving to the right, the mRNA and protein sequence, for a non-SNP subject. The sequence of the second codon is GTG (depicted by the open triangle) encoding for the amino acid Valine. Two Valine isoacceptor tRNA species are shown. Each of the two tRNA species recognize different Valine codons. The two species have different abundances. The species that recognize the wildtype codon, GTG, are not shaded and are in higher abundance. The shaded species, which has a lower abundance, does not pair with the wildtype codon. Thus, the Valine isoacceptor tRNA species that corresponds to the codon used (GTG) is abundant. This results in translation of the mRNA sequence into the corresponding protein as depicted. Using a more abundant tRNA species may also have an impact on transcript stability, protein expression, protein function, protein folding, or protein localization.

FIG. 3 The middle row depicts the endogenous tRNA pool and the mRNA and protein sequence for a subject having a single nucleotide polymorphism (SNP) at the third position of the second codon (shown with a closed triangle) in the depicted mRNA sequence. The composition of the endogenous tRNA pool is the same as described for FIG. 3 top row. However, incorporation of Valine at the second codon now depends on the use of a less abundant tRNA species (the shaded species). As a consequence, as shown in FIG. 3 middle row, translation of the mRNA sequence into the corresponding protein is compromised. Using a less abundant tRNA species may also reduce transcript stability, reduce protein expression, change protein function, change protein folding, or change protein localization.

FIG. 3 bottom row depicts the same mRNA sequence as in FIG. 3 middle row, which includes a SNP at the third position of the second codon (shown with a closed triangle). The endogenous tRNAs of the pool are the same as in the top row and the middle row, but the pool is supplemented with exogenous TREMs which increase the abundance of species that can pair with the SNP codon. As a consequence, translation of the mRNA sequence into the corresponding protein is not compromised.

FIG. 4 is a table provided a list of exemplary synonymous SNPs and related genes and diseases. The column titled “New codon” refers to the new codon created by the SNP. The columns titled “Fold change” and “Log 2 fold change” refer to the fold change/Log 2 fold change in codon usage based on genome codon usage, respectively.

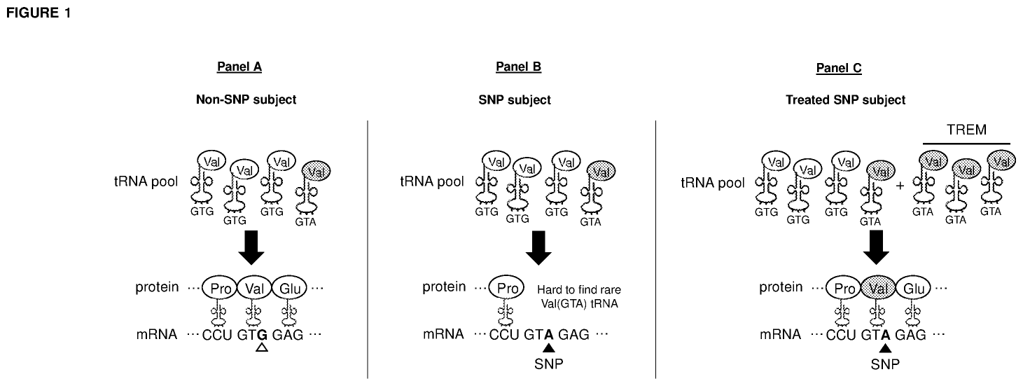

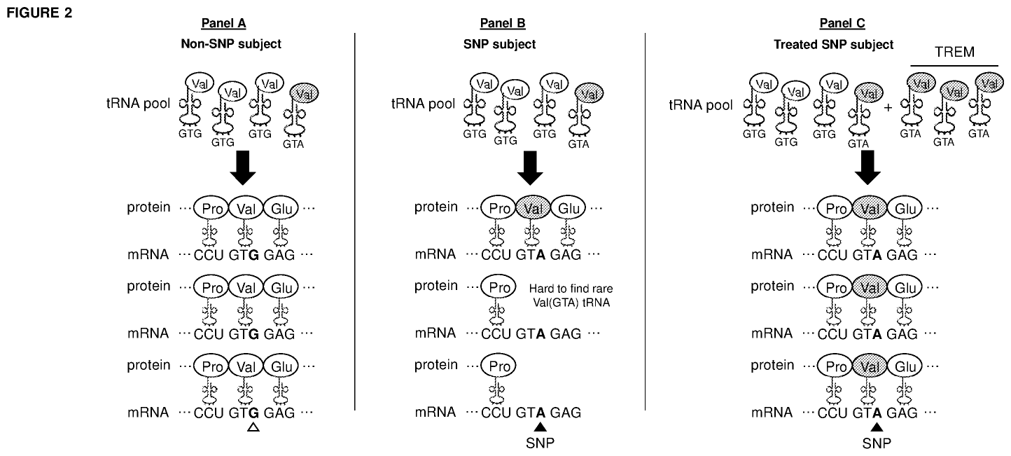

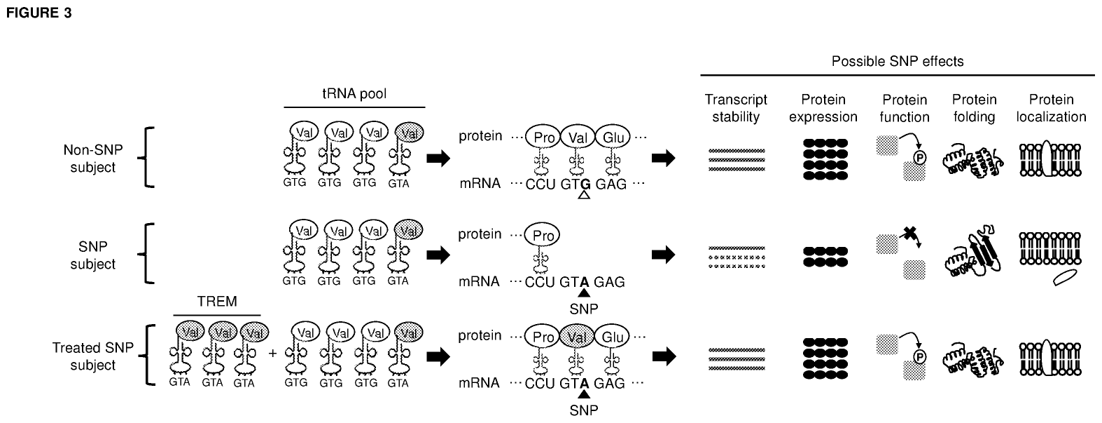

### DETAILED DESCRIPTION OF CERTAIN EMBODIMENTS

The present disclosure features uses of tRNA-based effector molecules (TREMs) comprising a non-naturally occurring modification to modulate tRNA pools in a cell or a subject. Also disclosed herein are methods of treating a disorder or ameliorating a symptom of a disorder by administering a composition comprising a TREM or a pharmaceutical composition comprising a TREM. Further disclosed herein are uses of TREMs comprising a non-naturally occurring modification to modulate a production parameter of an RNA, or a protein encoded by an RNA, wherein the RNA has a contextually-rare codon (“con-rare codon”).

As disclosed herein, TREMs are complex molecules which can mediate a variety of cellular processes. TREMs of the disclosure include TREMs, TREM core fragments and TREM fragments. TREMs, TREM core fragments or TREM fragments can be modified with non-naturally occurring modifications to, e.g., increase the level and/or activity (e.g., stability) of the TREM. Pharmaceutical TREM compositions, e.g., TREMs comprising a non-naturally occurring modification, can be administered to a cell, a tissue, or to a subject to modulate these functions, e.g., in vitro or in vivo. Disclosed herein are TREMs, TREM core fragments or TREM fragments comprising non-naturally occurring modifications, TREM compositions, preparations, methods of making TREM compositions and preparations, and methods of using the same.

### Definitions

A “contextually rare codon” or “con-rare codon”, as those terms are used herein, refer to a codon which, in a target cell or tissue, is limiting for a production parameter, e.g., an expression parameter, for a nucleic acid sequence having a con-rare codon (“con-rare codon nucleic acid sequence”), e.g., because the availability of a tRNA corresponding to the con-rare codon is limiting for a production parameter. Contextual rareness or con-rarity can be identified or evaluated by determining if the addition of a tRNA corresponding to a con-rare codon modulates, typically increases, a production parameter for a nucleic acid sequence, e.g., gene. By way of example, the method of Example 3, can be used, or adapted to be used, to evaluate con-rarity. Con-rarity as a property of a codon, is a function of, and can be identified or evaluated on the basis of, one, two, three, four, five, six, or all of the following factors:

(1) the sequence of the con-rare codon, or candidate con-rare codon;

(2) the availability of a corresponding tRNA for the con-rare, or candidate con-rare, -codon in a target cell or tissue Availability as a parameter can comprise or be a function of, one or both of the observed or predicted abundance or availability of a tRNA that corresponds to the con-rare codon. In an embodiment, abundance can be evaluated by quantifying tRNAs present in a target cell or tissue. See, e.g., Example 12;

(3) the contextual demand (the demand in a target cell or tissue) for a tRNA, e.g., a con-rare tRNA, or a candidate con-rare tRNA. This can be identified or evaluated by use of a parameter, a contextual demand-parameter, which comprises or is a function of, the demand or usage of a con-rare tRNA by one, some, or all of the nucleic acid sequences having con-rare codons in a target tissue or cell, e.g., the other nucleic acid sequences in a target cell or tissue which have a con-rare codon. A demand parameter can comprise of, or be a function of one or more, or all of:
(a) the expression profile (or proteomic properties) in the target cell or tissue (e.g., the abundance of expression) of one, some, or all of the nucleic acid sequences in the target cell or tissue which have a con-rare codon (e.g., for one or more, a subset of, or all of the expressed con-rare codon nucleic acid sequences in the target cell or tissue). In an embodiment, the expression profile (or proteomic properties) can be evaluated by evaluating proteins expressed in a target cell or tissue. See, e.g., Example 13;
(b) a measure which comprises or is a function of the frequency or proportion of appearance of the con-rare codon in an expressed nucleic acid sequence (e.g., for one or more, a subset of, or all of the expressed con-rare codon nucleic acid sequences in the target cell or tissue); or
(c) a parameter that is a function of (3)(a) and (3)(b);

(4) a parameter (or use-parameter) related to the con-rare codon usage in a con-rare codon nucleic acid sequence, and can include one or more of:
(a) the expression profile (or proteomic properties) in the target cell or tissue (e.g., the abundance of expression) of one, some, or all of the nucleic acid sequences in the target cell or tissue which have a con-rare codon, or a candidate nucleic acid sequence having a con-rare codon, (e.g., for one or more, a subset of, or all of the expressed con-rare codon nucleic acid sequences(s) in the target cell or tissue). In an embodiment, the expression profile (or proteomic properties) can be evaluated by evaluating proteins expressed in a target cell or tissue. See, e.g., Example 2;
(b) a measure which comprises or is a function of the frequency or proportion of appearance of the con-rare codon in a nucleic acid sequence having a con-rare codon (e.g., for one or more, a subset of, or all of the expressed con-rare codon nucleic acid sequence(s) in the target cell or tissue); or
(c) a parameter that is a function of (4)(a) and (4)(b);

(5) the proportion of the tRNAs corresponding to the con-rare codon which are charged;

(6) the iso-decoder isotype of the tRNA corresponding to the con-rare codon; and

(7) one or more post-transcriptional modifications of the con-rare tRNA, or candidate con-rare tRNA; and

In an embodiment, a con-optimized nucleic acid sequence has one less or one more con-rare codon than a reference sequence, e.g., a parental sequence, a naturally occurring sequence, a wildtype sequence, or a conventionally optimized sequence.

In an embodiment, con-rarity can be identified or evaluated by: (i) direct determination of whether a con-rare codon or candidate con-rare codon is limiting for a production parameter, e.g., in an assay analogous to that of Example 3; (ii) whether a con-rare or candidate con-rare codon meets a predetermined value, e.g., a standard or reference value (e.g., as described herein), of one or more, or all of factors (1)-(7); or (i) and (ii).

In an embodiment, con-rarity can be identified or evaluated by a production parameter, e.g., an expression parameter or a signaling parameter, e.g., as described herein.

In an embodiment, con-rarity is a function of normalized proteome codon count and tRNA abundance in a target tissue or cell. In an embodiment, con-rarity is a measure of codon frequency that is contextually dependent on tRNA abundance levels in a target tissue or cell.

Thus, the identification of a codon as a con-rare codon can involve a multi-parameter function of (1)-(7). In an embodiment, the con-rare codon meets a reference value for at least one of (1)-(7). In an embodiment, the con-rare codon meets a reference value for at least one of (1)-(7). In an embodiment, the con-rare codon meets a reference value for at least two of (1)-(7). In an embodiment, the con-rare codon meets a reference value for at least three of (1)-(7). In an embodiment, the con-rare codon meets a reference value for at least four of (1)-(7). In an embodiment, the con-rare codon meets a reference value for at least five of (1)-(7). In an embodiment, the con-rare codon meets a reference value for at least six of (1)-(7). In an embodiment, the con-rare codon meets a reference value for at all of (1)-(7). In an embodiment the reference value is a pre-determined or pre-selected value, e.g., as described herein.

In an embodiment, the identity of a con-rare codon is the DNA sequence which encodes for the codon in the nucleic acid sequence, e.g., gene.

In an embodiment, a con-rare codon is a function of the prevalence of the codon in the open reading frame (ORF) of protein coding genes in an organism, e.g., the proteome.

The availability, e.g., abundance, of tRNAs that correspond to a con-rare codon can be measured using an assay known in the art or as described herein, e.g., Nanopore sequencing, e.g., as described in Example 1. In an embodiment, a con-rare codon nucleic acid sequence has a low abundance of a tRNA corresponding to the con-rare codon, e.g., as compared to the abundance of a tRNA corresponding to a different/second codon.

The expression profile or proteomic property of a target cell or tissue refers to the protein expression, e.g., level of protein expression, from all of the protein coding genes in a target cell or tissue. The expression profile or proteomic property of a target cell or tissue can be measured using an assay known in the art or as described herein, e.g., a mass spectrometry based method, e.g., a SILAC based method as described in Example 13. In an embodiment, a protein coding gene in a target cell or tissue is a function of tissue or cell type specific regulation, e.g., a promoter element, an enhancer element, epigenetic regulation, and/or transcription factor control.

A “contextually-modified nucleic acid sequence” (sometimes referred to herein as a “con-modified nucleic acid sequence”) refers to a nucleic acid sequence in which the con-rarity of a codon of the con-modified nucleic acid sequence has been altered. E.g., a con-rare codon is replaced with a con-abundant codon and/or a con-abundant codon is replaced with a con-rare codon. In an embodiment, the con-modified nucleic acid sequence has one more or one less, e.g., two more or two lesser, con-rare codons, than a reference nucleic acid sequence. In an embodiment, the con-modified nucleic acid sequence has a codon with con-rarity that differs from the con-rarity of the corresponding codon in a reference nucleic acid sequence.

The reference nucleic acid sequence can be, e.g., any selected sequence, a parental sequence, a starting sequence, a wildtype or naturally occurring sequence that encodes the same amino acid at the corresponding codon, a wildtype or naturally occurring sequence that encodes the same polypeptide, or a conventionally codon-optimized sequence. In an embodiment, the reference nucleic acid sequence encodes the same polypeptide sequence as the con-modified nucleic acid sequence. In an embodiment, the reference nucleic acid sequence encodes a polypeptide sequence that differs from the con-modified nucleic acid sequence at a position other than the con-rare modified sequence. In an embodiment, a con-modified nucleic acid sequence results in a different production parameter, e.g., an expression parameter or signaling parameter, compared to that seen with expression of a reference nucleic acid sequence.

In an embodiment, a con-modified nucleic acid sequence refers to a nucleic acid sequence which has one more or one less, e.g., two more or two lesser, con-rare codons, than a reference sequence, wherein the con-modified nucleic acid sequence encodes a polypeptide that comprises the reference sequence.

A “contextually-rare tRNA” or “con-rare tRNA,” is a tRNA that corresponds to a con-rare codon.

A “contextually-abundant codon” or “con-abundant codon” as those terms are used herein, refer to a codon other than a con-rare codon.

A “con-rare codon nucleic acid sequence,” or a “nucleic acid sequence having a con-rare codon” as those terms are used herein, refer to a nucleic acid sequence, e.g., DNA, or RNA, or gene, comprising a con-rare codon. In an embodiment, in such con-rare codon nucleic acid sequences, modulation of a production parameter, e.g., an expression parameter or signaling parameter, can be mediated by altering the availability, e.g., abundance of a con-rare tRNA. In an embodiment, the con-rare codon is in a translated region of the con-rare codon nucleic acid sequence, e.g., in an open reading frame (ORF) or coding sequence (CDS).

A “con-rare codon RNA,” as that term is used herein, refers to an RNA sequence comprising a con-rare codon. In an embodiment, a con-rare codon RNA comprises a messenger RNA or an RNA that can be translated into a polypeptide or protein. In an embodiment, a con-rare codon RNA is transcribed from a complementary DNA sequence which comprises said con-rare codon. In an embodiment, the con-rare codon RNA is transcribed in vivo. In an embodiment, the con-rare codon RNA is transcribed in vitro.

A “codon-value” as that term is used herein, is a function of the con-rarity of a sequence-codon in a sequence. Con-rarity of a codon is a function of one or more factors as described in the definition of “con-rare codon” above. In an embodiment, a codon-value is the identity of a codon, e.g., a replacement codon selected to replace the sequence-codon. In an embodiment, when the replacement codon is a con-abundant codon, the sequence codon is a con-rare codon. In an embodiment, when the replacement codon is a con-rare codon, the sequence-codon is a con-abundant codon.

A “sequence-codon” as that term is used herein, refers to a codon in a nucleic acid sequence for which a codon-value is acquired.

A “production parameter,” refers to an expression parameter and/or a signaling parameter. In an embodiment a production parameter is an expression parameter. An expression parameter includes an expression parameter of a polypeptide or protein encoded by the con-rare codon nucleic acid sequence; or an expression parameter of an RNA, e.g., messenger RNA, encoded by the con-rare codon nucleic acid sequence. In an embodiment, an expression parameter can include:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

In an embodiment, a production parameter is a signaling parameter. A signaling parameter can include:

(1) modulation of a signaling pathway, e.g., a cellular signaling pathway which is downstream or upstream of the protein encoded by the con-rare codon nucleic acid sequence;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

An “isoacceptor,” as that term is used herein, refers to a plurality of tRNA molecule or TREMs wherein each molecule of the plurality comprises a different naturally occurring anticodon sequence and each molecule of the plurality mediates the incorporation of the same amino acid and that amino acid is the amino acid that naturally corresponds to the anticodons of the plurality.

A “tRNA pool,” as that term is used herein, refers to the pool of all species, e.g., endogenous tRNAs and TREMS, which can function as tRNAs. The endogenous tRNA pool for a cell or subject that has not been administered a TREM includes only endogenous tRNAs. A TREM can be added to modulate a tRNA pool comprising only endogenous tRNAs, but can also be administered to a cell or subject that has a tRNA pool that includes TREMs that have been administered previously. In an embodiment, the TREM which is administered to a cell or a subject, mediates initiation or elongation by incorporating the amino acid (the cognate amino acid) associated in nature with a particular anticodon. In an embodiment, the TREM which is administered has an anticodon other than a stop codon.

A “nucleotide,” as that term is used herein, refers to an entity comprising a sugar, typically a pentameric sugar; a nucleobase; and a phosphate linking group. In an embodiment, a nucleotide comprises a naturally occurring, e.g., naturally occurring in a human cell, nucleotide, e.g., an adenine, thymine, guanine, cytosine, or uracil nucleotide.

A “modification,” as that term is used herein with reference to a nucleotide, refers to a modification of the chemical structure, e.g., a covalent modification, of the subject nucleotide. The modification can be naturally occurring or non-naturally occurring. In an embodiment, the modification is non-naturally occurring. In an embodiment, the modification is naturally occurring. In an embodiment, the modification is a synthetic modification. In an embodiment, the modification is a modification provided in Tables 5, 6, 7, 8 or 9.

A “non-naturally occurring modification,” as that term is used herein with reference to a nucleotide, refers to a modification that: (a) a cell, e.g., a human cell, does not make on an endogenous tRNA; or (b) a cell, e.g., a human cell, can make on an endogenous tRNA but wherein such modification is in a location in which it does not occur on a native tRNA, e.g., the modification is in a domain, linker or arm, or on a nucleotide and/or at a position within a domain, linker or arm, which does not have such modification in nature. In either case, the modification is added synthetically, e.g., in a cell free reaction, e.g., in a solid state or liquid phase synthetic reaction. In an embodiment, the non-naturally occurring modification is a modification that is not present (in identity, location or position) if a sequence of the TREM is expressed in a mammalian cell, e.g., a HEK293 cell line. Exemplary non-naturally occurring modifications are found in Tables 5, 6, 7, 8 or 9.

A “non-naturally modified nucleotide,” as that term is used herein, refers a nucleotide comprising a non-naturally occurring modification on or of a sugar, nucleobase, or phosphate moiety.

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

A “naturally occurring nucleotide,” as that term is used herein, refers to a nucleotide that does not comprise a non-naturally occurring modification. In an embodiment, it includes a naturally occurring modification.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. The TREMs described in the present invention are synthetic molecules and are made, e.g., in a cell free reaction, e.g., in a solid state or liquid phase synthetic reaction. TREMs are chemically distinct, e.g., in terms of primary sequence, type or location of modifications from the endogenous tRNA molecules made in cells, e.g., in mammalian cells, e.g., in human cells. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:

(a′) an optional linker region of a consensus sequence provided in the “Consensus Sequence” section, e.g., a Linker 1 region;

(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 4. In an embodiment, the TREM can comprise a fragment or analog of an AStD, e.g., an AStD encoded by a nucleic acid in Table 4, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 4. E.g., one of ordinary skill can determine the sequence which corresponds to an AStD from a tRNA sequence encoded by a nucleic acid in Table 4.)

In an embodiment the AStD falls under the corresponding sequence of a consensus sequence provided in the “Consensus Sequence” section, or differs from the consensus sequence by no more than 1, 2, 5, or 10 positions;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I_{ZZZ}, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II_{ZZZ}, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III_{ZZZ}, wherein ZZZ indicates any of the twenty amino acids;

(a′-1) a linker comprising residues R₈-R₉ of a consensus sequence provided in the “Consensus Sequence” section, e.g., a Linker 2 region;

(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 4. In an embodiment, the TREM can comprise a fragment or analog of a DHD, e.g., a DHD encoded by a nucleic acid in Table 4, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula I_{ZZZ}, wherein ZZZ indicates any of the twenty amino acids;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula II_{ZZZ}, wherein ZZZ indicates any of the twenty amino acids;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula III_{ZZZ}, wherein ZZZ indicates any of the twenty amino acids;

(b′-1) a linker comprising residue R₂₉ of a consensus sequence provided in the “Consensus Sequence” section, e.g., a Linker 3 region;

(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 4. In an embodiment, the TREM can comprise a fragment or analog of an ACHD, e.g., an ACHD encoded by a nucleic acid in Table 4, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;

In an embodiment, the ACHD comprises residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I_{ZZZ}, wherein ZZZ indicates any of the twenty amino acids;

In an embodiment, the ACHD comprises residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula II_{ZZZ}, wherein ZZZ indicates any of the twenty amino acids;

In an embodiment, the ACHD comprises residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III_{ZZZ}, wherein ZZZ indicates any of the twenty amino acids;

(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 4. In an embodiment, the TREM can comprise a fragment or analog of a VLD, e.g., a VLD encoded by a nucleic acid in Table 4, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue —[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);

(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 4. In an embodiment, the TREM can comprise a fragment or analog of a THD, e.g., a THD encoded by a nucleic acid in Table 4, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;

In an embodiment, the THD comprises residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I_{ZZZ}, wherein ZZZ indicates any of the twenty amino acids;

In an embodiment, the THD comprises residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II_{ZZZ}, wherein ZZZ indicates any of the twenty amino acids;

In an embodiment, the THD comprises residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula III_{ZZZ}, wherein ZZZ indicates any of the twenty amino acids;

(e′ 1) a linker comprising residue R₇₂ of a consensus sequence provided in the “Consensus Sequence” section, e.g., a Linker 4 region;

(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 4. In an embodiment, the TREM can comprise a fragment or analog of a stem or loop structure, e.g., a stem or loop structure encoded by a nucleic acid in Table 4, which fragment in embodiments has activity of a stem or loop structure, and in other embodiments does not have activity of a stem or loop structure;

(g) a tertiary structure, e.g., an L-shaped tertiary structure;

(h) adaptor function, i.e., the TREM mediates acceptance of an amino acid, e.g., its cognate amino acid and transfer of the AA in the initiation or elongation of a polypeptide chain;

(i) cognate adaptor function wherein the TREM mediates acceptance and incorporation of an amino acid (e.g., cognate amino acid) associated in nature with the anti-codon of the TREM to initiate or elongate a polypeptide chain;

(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;

(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;

(l) a structure which allows for ribosome binding;

(m) a post-transcriptional modification, e.g., a naturally occurring post-transcriptional modification;

(n) the ability to inhibit a functional property of a tRNA, e.g., any of properties (h)-(k) possessed by a tRNA;

(o) the ability to modulate cell fate;

(p) the ability to modulate ribosome occupancy;

(q) the ability to modulate protein translation;

(r) the ability to modulate mRNA stability;

(s) the ability to modulate protein folding and structure;

(t) the ability to modulate protein transduction or compartmentalization;

(u) the ability to modulate protein stability; or

(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:

(i) an amino acid attachment domain that binds an amino acid (e.g., an AStD, as described in (a) herein; and

(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 4, or a fragment or functional fragment thereof. In an embodiment, a TREM comprises an RNA sequence encoded by a DNA sequence listed in Table 4, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 4, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 4, or a fragment or a functional fragment thereof. In an embodiment, a TREM comprises a TREM domain, e.g., a domain described herein, comprising an RNA sequence encoded by DNA sequence listed in Table 4, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 4, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′ halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM core fragment,” as that term is used herein, refers to a portion of the sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1.

A “TREM fragment,” as used herein, refers to a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

“Post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM.

A “subject,” as this term is used herein, includes any organism, such as a human or other animal. In embodiments, the subject is a vertebrate animal (e.g., mammal, bird, fish, reptile, or amphibian). In embodiments, the subject is a mammal, e.g., a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). The subject may be a male or female of any age group, e.g., a pediatric subject (e.g., infant, child, adolescent) or adult subject (e.g., young adult, middle-aged adult, or senior adult)). A non-human subject may be a transgenic animal.

A “synthetic TREM,” as that term is used herein, refers to a TREM which was synthesized other than in or by a cell having an endogenous nucleic acid encoding the TREM, e.g., a synthetic TREM is synthetized by cell-free solid phase synthesis. A synthetic TREM can have the same, or a different, sequence, or tertiary structure, as a native tRNA.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “tRNA”, as that term is used herein, refers to a naturally occurring transfer ribonucleic acid in its native state.

A “TREM composition,” as that term is used herein, refers to a composition comprising a plurality of TREMs, a plurality of TREM core fragments and/or a plurality of TREM fragments. A TREM composition can comprise one or more species of TREMs, TREM core fragments or TREM fragments. In an embodiment, the composition comprises only a single species of TREM, TREM core fragment or TREM fragment. In an embodiment, the TREM composition comprises a first TREM, TREM core fragment or TREM fragment species; and a second TREM, TREM core fragment or TREM fragment species. In an embodiment, the TREM composition comprises X TREM, TREM core fragment or TREM fragment species, wherein X=2, 3, 4, 5, 6, 7, 8, 9, or 10. In an embodiment, the TREM, TREM core fragment or TREM fragment has at least 70, 75, 80, 85, 90, or 95, or has 100%, identity with a sequence encoded by a nucleic acid in Table 4. A TREM composition can comprise one or more species of TREMs, TREM core fragments or TREM fragments. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

In an embodiment, at least X % of the TREMs in a TREM composition has a non-naturally occurring modification at a selected position, and X is 80, 90, 95, 96, 97, 98, 99, or 99.5.

In an embodiment, at least X % of the TREMs in a TREM composition has a non-naturally occurring modification at a first position and a non-naturally occurring modification at a second position, and X, independently, is 80, 90, 95, 96, 97, 98, 99, or 99.5. In embodiments, the modification at the first and second position is the same. In embodiments, the modification at the first and second position are different. In embodiments, the nucleotide at the first and second position is the same, e.g., both are adenine. In embodiments, the nucleotide at the first and second position are different, e.g., one is adenine and one is thymine.

In an embodiment, at least X % of the TREMs in a TREM composition has a non-naturally occurring modification at a first position and less than Y % have a non-naturally occurring modification at a second position, wherein X is 80, 90, 95, 96, 97, 98, 99, or 99.5 and Y is 20, 20, 5, 2, 1, 0.1, or 0.01. In embodiments, the nucleotide at the first and second position is the same, e.g., both are adenine. In embodiments the nucleotide at the first and second position are different, e.g., one is adenine and one is thymine.

“Pairs with” or “pairing,” as those terms are used herein, refer to the correspondence of a codon with an anticodon and includes fully complementary codon:anticodon pairs as well as “wobble” pairing, in which the third position need not be complementary. Fully complementary pairing refers to pairing of all three positions of the codon with the corresponding anticodon according to Watson-Crick base pairing. Wobble pairing refers to complementary pairing of the first and second positions of the codon with the corresponding anticodon according to Watson-Crick base pairing, and flexible pairing at the third position of the codon with the corresponding anticodon.

The terms modified, replace, derived and similar terms, when used or applied in reference to a product, refer only to the end product or structure of the end product, and are not restricted by any method of making or manufacturing the product, unless expressly provided as such in this disclosure.

Headings, titles, subtitles, numbering or other alpha/numeric hierarchies are included merely for ease of reading and absent explicit language to the contrary do not indicate order of performance, order of importance, magnitude or other value.

### Contextually-Rare Codons (“Con-Rare Codons”)

Disclosed herein, inter alia, is the observation that a production parameter of an RNA, or a protein encoded by an RNA having a con-rare codon, can be modulated by administration of a TREM composition comprising a TREM, TREM core fragment or TREM fragment, e.g., as described herein, corresponding to said con-rare codon. Accordingly, this disclosure provides, inter alia, methods of identifying a contextually rare codon (“con-rare codon”), compositions of TREMs corresponding to a con-rare codon and uses of said TREM compositions.

A con-rare codon is a codon that is limiting for a production parameter, e.g., an expression parameter or a signaling parameter, for a nucleic acid sequence, e.g., a DNA or an RNA, or a protein encoded by a nucleic acid sequence, e.g., a DNA or an RNA. Contextual rareness or con-rarity can be identified or evaluated by determining if the addition of a tRNA corresponding to a con-rare codon modulates, typically increases, a production parameter for a target nucleic acid sequence, e.g., target, e.g., gene. In an embodiment, con-rarity as a property of a codon, is a function of, one, two, three, four, all of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon.

In an embodiment, con-rarity is a function of normalized proteome codon count and tRNA abundance in a target tissue or cell. In an embodiment, con-rarity is a measure of codon frequency that is contextually dependent on tRNA abundance levels in a target tissue or cell. In an embodiment, con-rarity can be identified or evaluated by a production parameter, e.g., an expression parameter or a signaling parameter, e.g., as described herein.

An exemplary method of evaluating con-rarity and identifying a con-rare codon is provided in Example 3.

### Exemplary Reference Values for Evaluating Con-Rarity

In an embodiment, con-rarity is a function of normalized proteome codon count and the tRNA profile, e.g., as described herein. In an embodiment, con-rarity is determined by dividing the normalized proteome codon count by the tRNA profile determined by Nanopore or other tRNA sequencing experiment. This provides a measure of codon usage that is contextually dependent on the tRNA profile, e.g., tRNA abundance levels.

In an embodiment, a codon is determined to be contextually rare (con-rare) if the con-rarity meets a reference value, e.g., a pre-determined or pre-selected reference value, e.g., a threshold, e.g., an internal threshold, e.g., as described herein. In an embodiment, the reference value is a value under which e.g., 1.5× sigma of the normally fit distribution to that codon frequency.

In an embodiment, a codon is con-rare if the value of a normalized proteome codon count divided by the tRNA profile value for a particular tRNA meets a reference value, e.g., a pre-determined or pre-selected reference value, e.g., a threshold, e.g., an internal threshold.

In an embodiment, a codon is con-rare if the value of a normalized proteome codon count divided by the tRNA profile value for a particular tRNA is in the top 5%, 10%, 20%, 30%, or 40% of values for normalized proteome codon count divided by the tRNA profile value for all codons measured, e.g., wherein all 64 codons are measured. In an embodiment, a codon is con-rare if the value of a normalized proteome codon count divided by the tRNA profile value for a particular tRNA is in the top 5% of values for normalized proteome codon count divided by the tRNA profile value for all codons measured. In an embodiment, a codon is con-rare if the value of a normalized proteome codon count divided by the tRNA profile value for a particular tRNA is in the top 10% of values for normalized proteome codon count divided by the tRNA profile value for all codons measured. In an embodiment, a codon is con-rare if the value of a normalized proteome codon count divided by the tRNA profile value for a particular tRNA is in the top 20% of values for normalized proteome codon count divided by the tRNA profile value for all codons measured. In an embodiment, a codon is con-rare if the value of a normalized proteome codon count divided by the tRNA profile value for a particular tRNA is in the top 30% of values for normalized proteome codon count divided by the tRNA profile value for all codons measured. In an embodiment, a codon is con-rare if the value of a normalized proteome codon count divided by the tRNA profile value for a particular tRNA is in the top 40% of values for normalized proteome codon count divided by the tRNA profile value for all codons measured.

In an embodiment, a codon is con-rare if for the value of a normalized proteome codon count divided by the tRNA profile value for a particular tRNA, the value for the normalized proteome codon count is below the value for all codons measured and the value for tRNA profile, is above the value for all codons measured, e.g., wherein all 64 codons are measured.

In an embodiment, a codon is a con-rare codon if it is in the upper left quadrant of a plot of normalized proteome codon count (y-axis) vs tRNA profile (x-axis), with equal number of codons in each quadrant, e.g., wherein all 64 codons are measured.

In an embodiment, a codon is a con-rare codon if it is in a quadrant other than the lower right quadrant of a plot of normalized proteome codon count (y-axis) vs tRNA profile (x-axis), with equal number of codons in each quadrant, e.g., wherein all 64 codons are measured.

Methods of Modulating a Production Parameter of an RNA, or a Protein Encoded by an RNA Having a Con-Rare Codon with a TREM Composition

A production parameter of an RNA, or a protein encoded by an RNA having a con-rare codon, can be modulated by administration of a TREM composition comprising a TREM, a TREM core fragment, or TREM fragment, e.g., as described herein, corresponding to said con-rare codon.

In an aspect, provided herein is a method of method of modulating a production parameter of an RNA, or a protein encoded by an RNA, in a target cell or tissue, comprising:

providing, e.g., administering, to the target cell or tissue, or contacting the target cell or tissue with, an effective amount of a TREM composition comprising a TREM, a TREM core fragment, or TREM fragment, which TREM, TREM core fragment or TREM fragment corresponds to a contextually-rare codon (“con-rare codon”) of the RNA,

thereby modulating the production parameter of the RNA, or protein encoded by the RNA in the target cell or tissue.

The TREM composition can be administered to the subject or the target cell or tissue can be contacted ex vivo with the TREM composition. In an embodiment, the target cell or tissue which has been contacted ex vivo with the TREM composition can be introduced into a subject, e.g., an allogeneic subject or an autologous subject.

Modulation of a production parameter of an RNA, or a protein encoded by an RNA having a con-rare codon by administration of a TREM composition (e.g., comprising a TREM, TREM core fragment or TREM fragment corresponding to the con-rare codon) comprises modulation of an expression parameter or a signaling parameter, e.g., as described herein.

For example, administration of a TREM composition to a target cell or tissue can result in an increase or decrease in any one or more of the following expression parameters for the con-rare codon RNA:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

As another example, administration of a TREM composition to a target cell or tissue can result in an increase or decrease in any one or more of the following signaling parameters for the con-rare codon RNA:

(1) modulation of a signaling pathway, e.g., a cellular signaling pathway which is downstream or upstream of the protein encoded by the con-rare codon RNA;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

A production parameter (e.g., an expression parameter and/or a signaling parameter) may be modulated, e.g., by at least 5% (e.g., at least 10%, 15%, 20%, 25%, 30%, 40%. 50%. 60%. 70%, 80%, 90%, 100%, 150%, 200% or more) compared to a reference nucleic acid sequence, e.g., parental, wildtype or conventionally optimized nucleic acid sequence.

Synonymous SNPs and Method of Modulating tRNA Pool

A single nucleotide polymorphism (SNP) is a mutation that is found in the genome. A SNP can occur anywhere in the genome, e.g., in a coding sequence (e.g., an exon), or in a regulatory region (e.g., in an intron, a promoter element, an enhancer), or in a non-coding sequence.

A SNP that occurs in a coding sequence, e.g., an exon, can affect the corresponding polypeptide by altering a codon to specify a different amino acid, e.g., a different amino acid compared to that specified by the non-mutated codon.

A SNP that occurs in a coding sequence which alters a codon but does not change the amino acid specified by said mutated codon will not change the amino acid that is incorporated into the corresponding polypeptide at that position. This is possible due to the degeneracy of the genetic codon (i.e. more than one codon specifying one amino acid). Codon degeneracy is supported by “wobble” base pairing at the first base of the tRNA anticodon. For example, if a wildtype CTT codon which specifies the amino acid leucine is mutated to a CTC codon which specifies the same amino acid Leucine, no change to the corresponding protein with respect to its composition at that particular position is expected. Both codons CTT and CTC are recognized by tRNAs that specify the amino acid Leucine. These different species of tRNAs are referred to as isoacceptor tRNAs.

A mutation which changes a codon but does not change the corresponding amino acid specified by the mutated codon is called a synonymous SNP. Synonymous SNPs are also known as silent SNPs.

Synonymous SNPs found in the human population are linked to certain diseases. Since synonymous SNPs are not expected to alter the composition of the polypeptide chain, without wishing to be bound by theory, is it believed that the effect of a synonymous SNP is linked to bias in codon usage. For example, a synonymous SNP may result in reduced protein translation, altered protein folding, altered protein localization or altered protein function. The relationship between codon usage and tRNA abundance is currently being investigated.

In an embodiment, the amount of a tRNA in a cell is correlated with codon usage. In an embodiment, a tRNA which pairs with a codon that is highly used is more abundant than a tRNA which pairs with a codon that is not highly used. In an embodiment, a tRNA which pairs with a codon that is not highly used is less abundant than a tRNA which pairs with a codon that is highly used.

As defined herein, the tRNA pool in a cell is the tRNA pool of all species, e.g., endogenous tRNAs and TREMS, which can function as tRNAs. The endogenous tRNA pool for a cell or subject that has not been administered a TREM includes only endogenous tRNAs. The tRNA pool for a cell or subject that has been administered a TREM includes endogenous tRNAs and the TREM.

Without wishing to be bound by theory, it is believed that the tRNA pool in a cell or subject can be altered by administering a composition comprising a TREM to the cell or subject. In an embodiment, the tRNA pool in a cell or subject that has been administered a Composition comprising a TREM comprises endogenous tRNAs and the administered TREM.

In an aspect, disclosed herein is a TREM composition comprising a TREM, a TREM core fragment or TREM fragment (e.g., a pharmaceutical composition comprising a TREM as described herein) for use in modulating a tRNA pool in a cell or subject, e.g., as described herein.

In embodiments, a TREM composition (e.g., a pharmaceutical composition comprising a TREM) described herein is contacted with a cell or tissue, or administered to a subject in need thereof, in an amount and for a time sufficient to modulate (increase or decrease) the tRNA pool. In embodiments, the tRNA pool comprises a first tRNA moiety and an additional tRNA moiety, e.g., a second tRNA moiety. In an embodiment, a tRNA moiety comprises an endogenous tRNA and/or a TREM.

In an embodiment, TREM composition described herein (e.g., a pharmaceutical composition comprising a TREM as described herein) can be used to treat a subject having an endogenous ORF comprising a codon comprising a synonymous mutation (a synonymous mutation codon or SMC).

A TREM composition (e.g., a pharmaceutical composition comprising a TREM as described herein) can also be used to modulate a function in a cell, tissue or subject. In embodiments, a TREM composition (e.g., a pharmaceutical composition comprising a TREM) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein localization; protein folding; protein stability; protein transduction; or protein compartmentalization.

A parameter may be modulated, e.g., by at least 5% (e.g., at least 10%, 15%, 20%, 25%, 30%, 40%. 50%. 60%. 70%, 80%, 90%, 100%, 150%, 200% or more) compared to a reference tissue, cell or subject (e.g., a healthy, wild-type or control cell, tissue or subject).

In an embodiment, a subject or a cell having a synonymous SNP has a tRNA pool which has a lower abundance of the tRNA that pairs with the SNP codon. In an embodiment, administration of a TREM that pairs with the SNP codon to the subject or cell, increases the amount of the isoaccepting tRNA pool in the subject or cell, e.g., increase the amount of amino acid specifying molecule that can pair with the SNP codon.

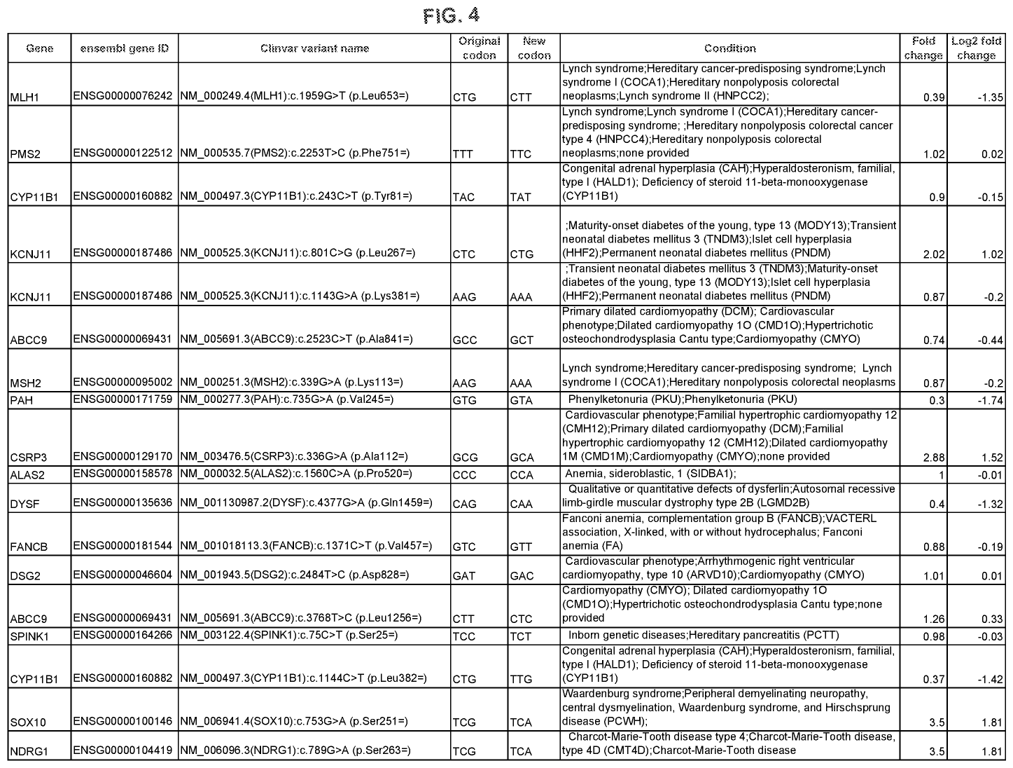

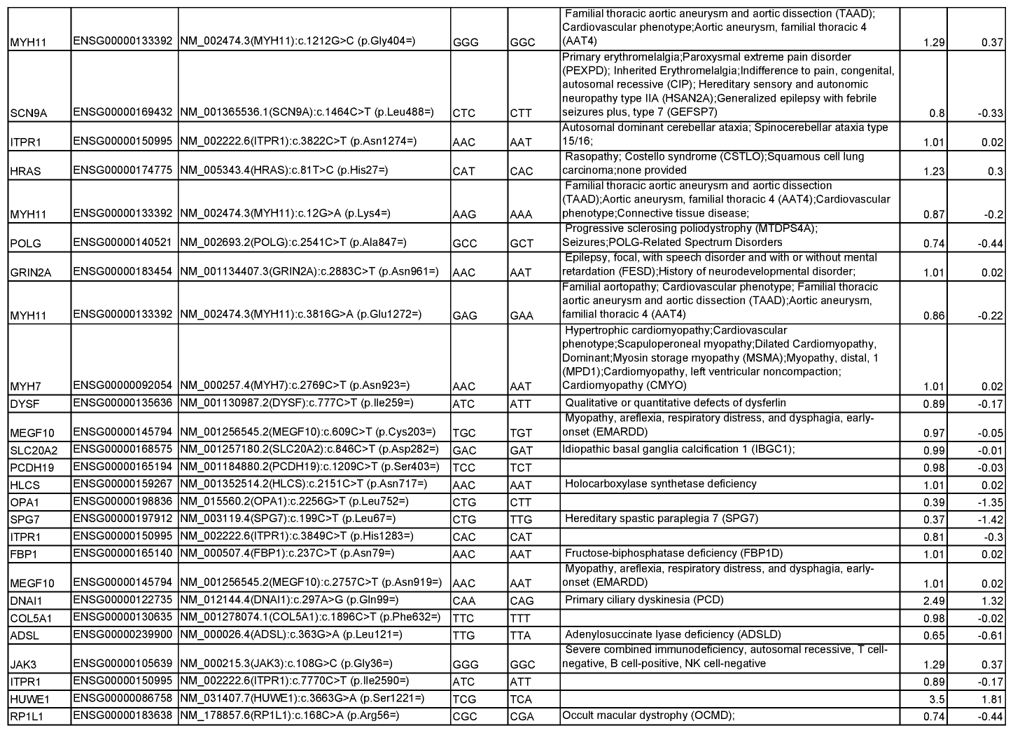

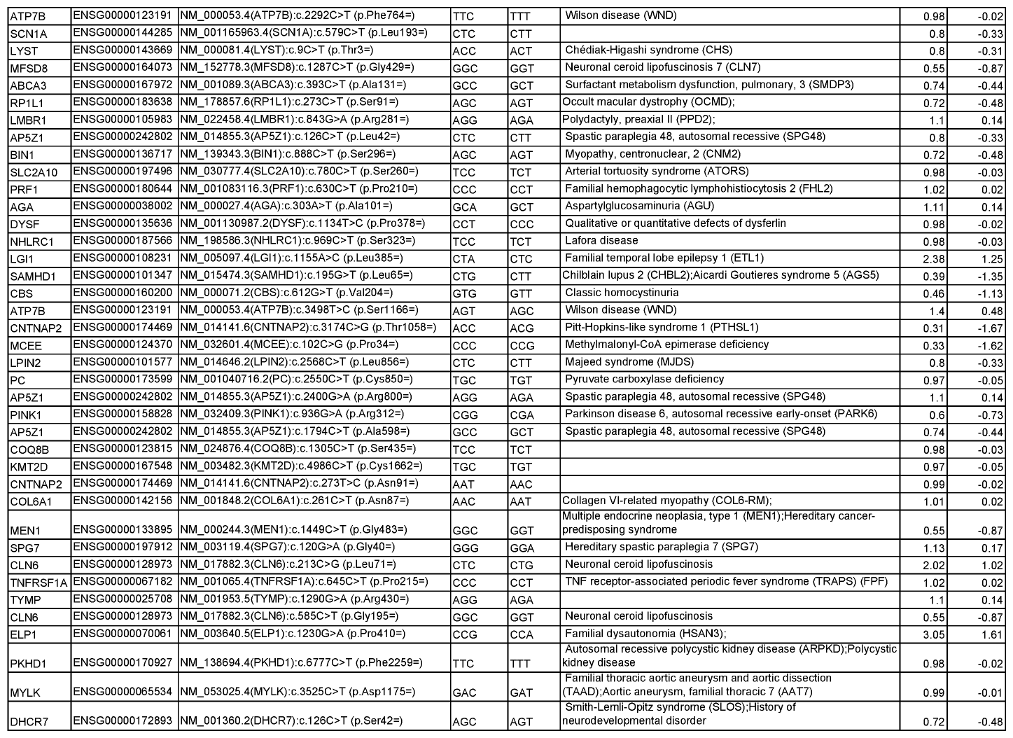

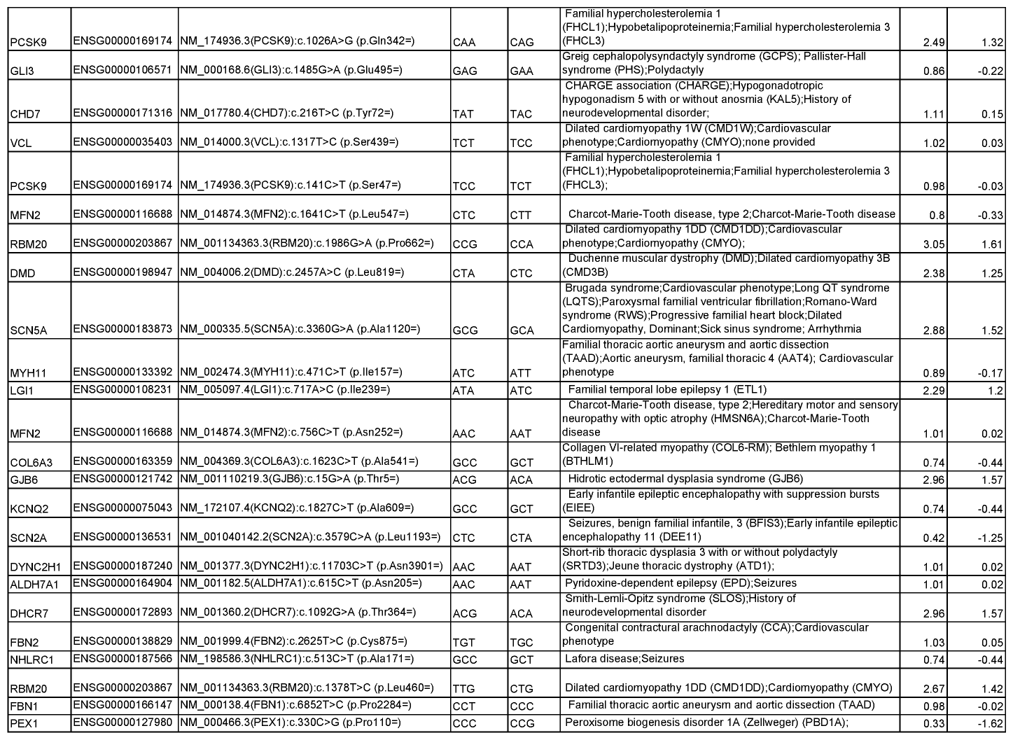

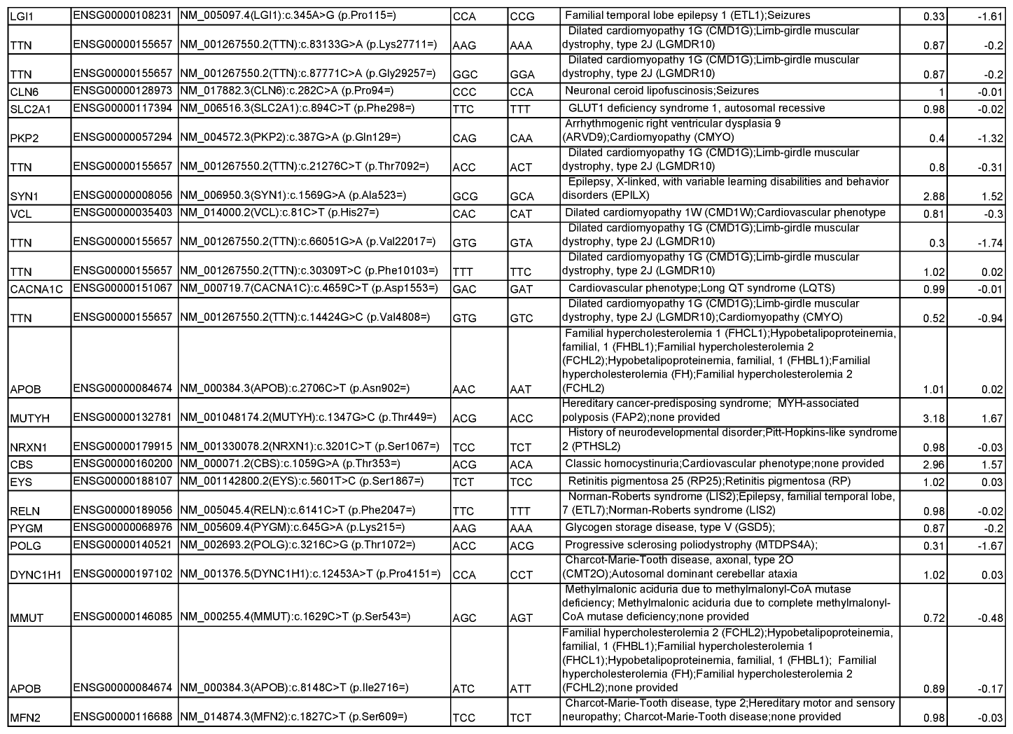

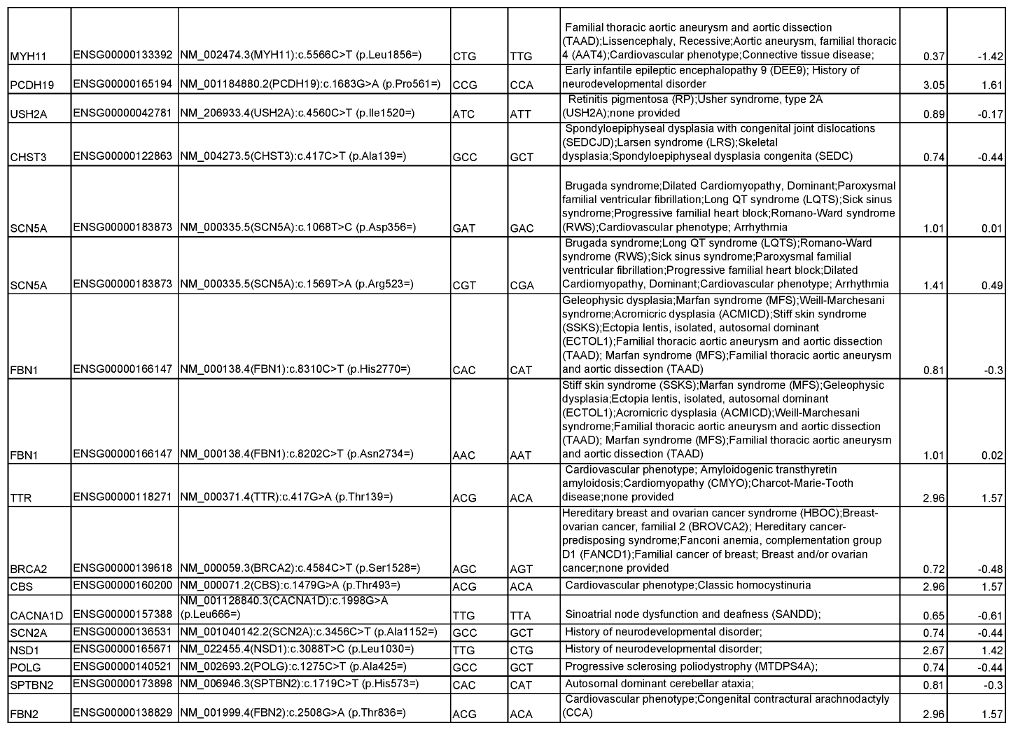

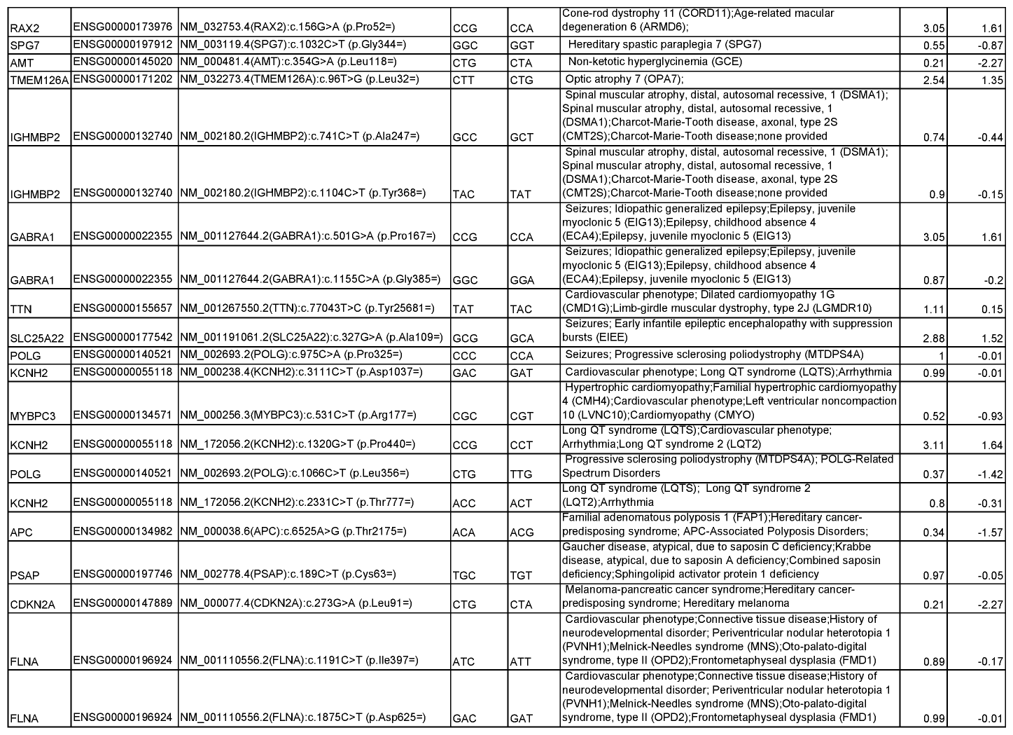

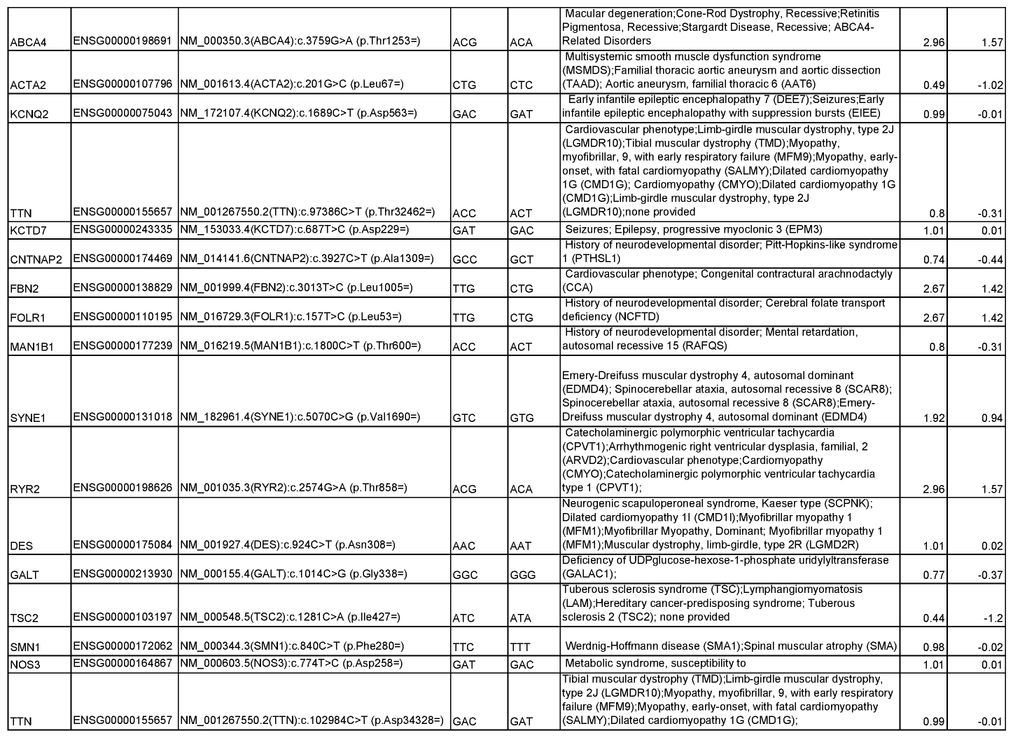

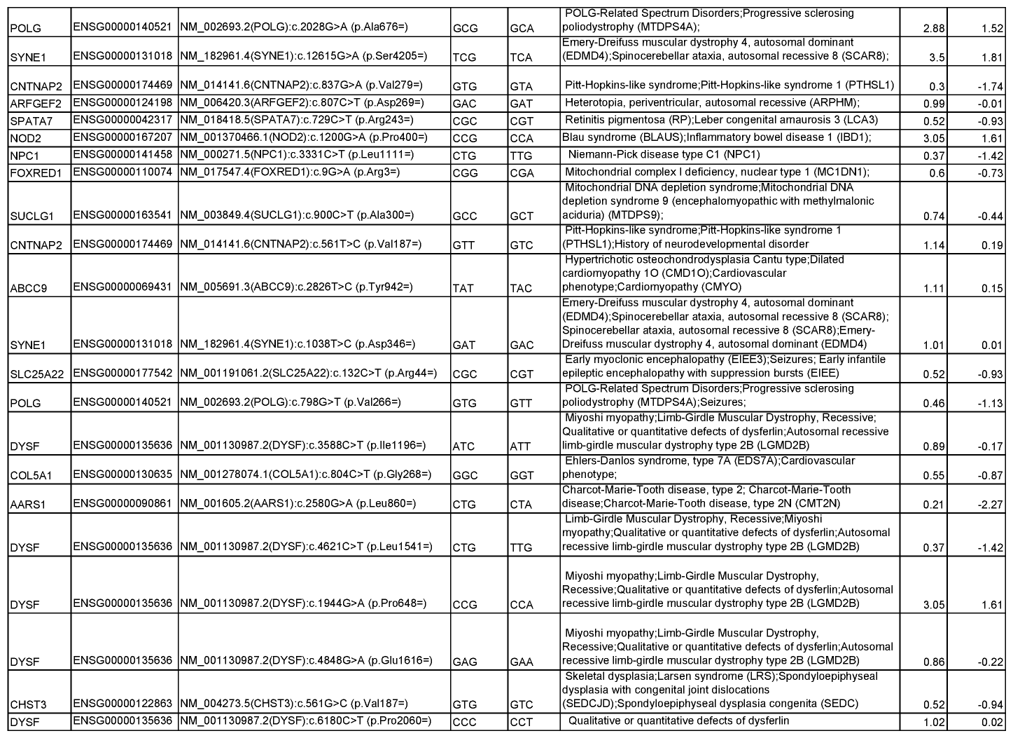

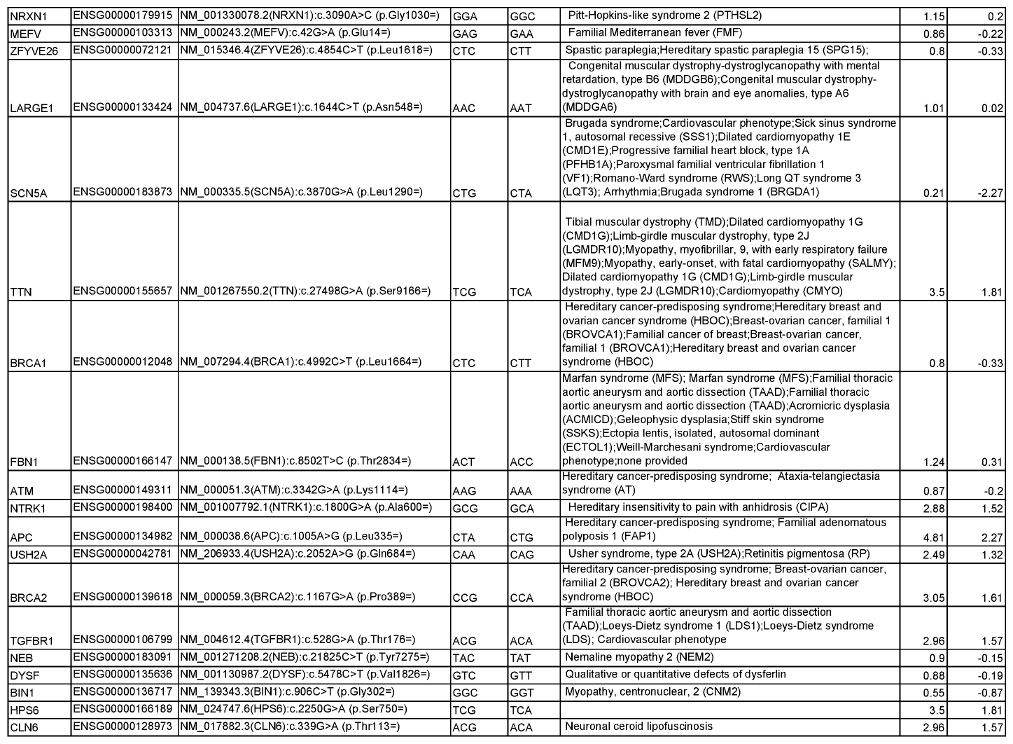

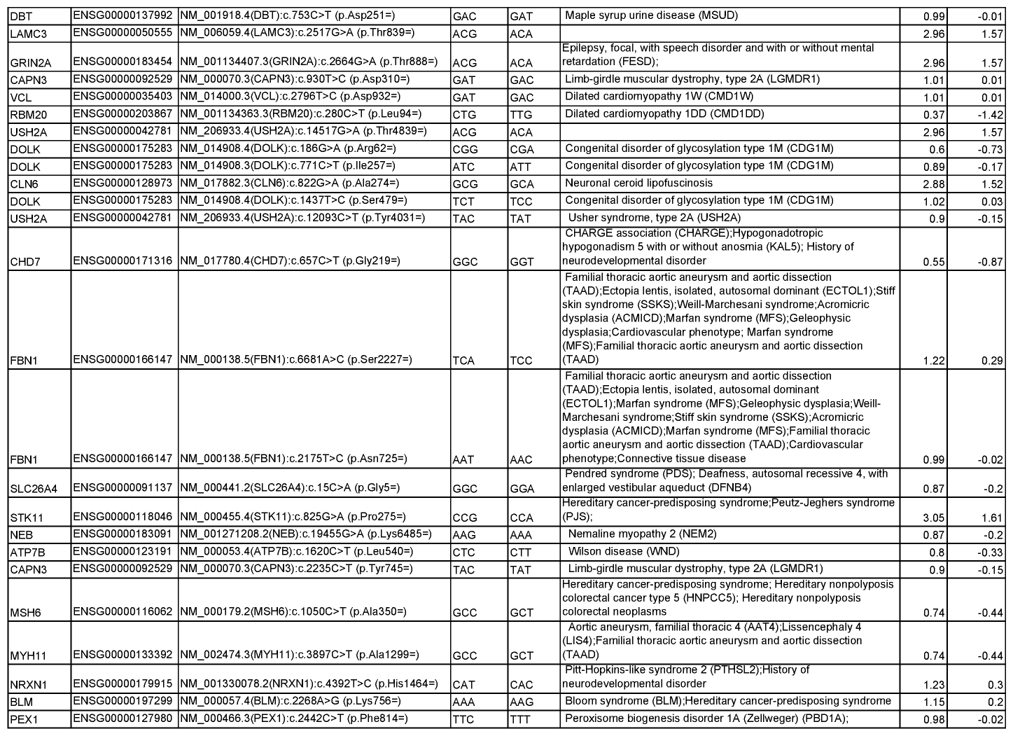

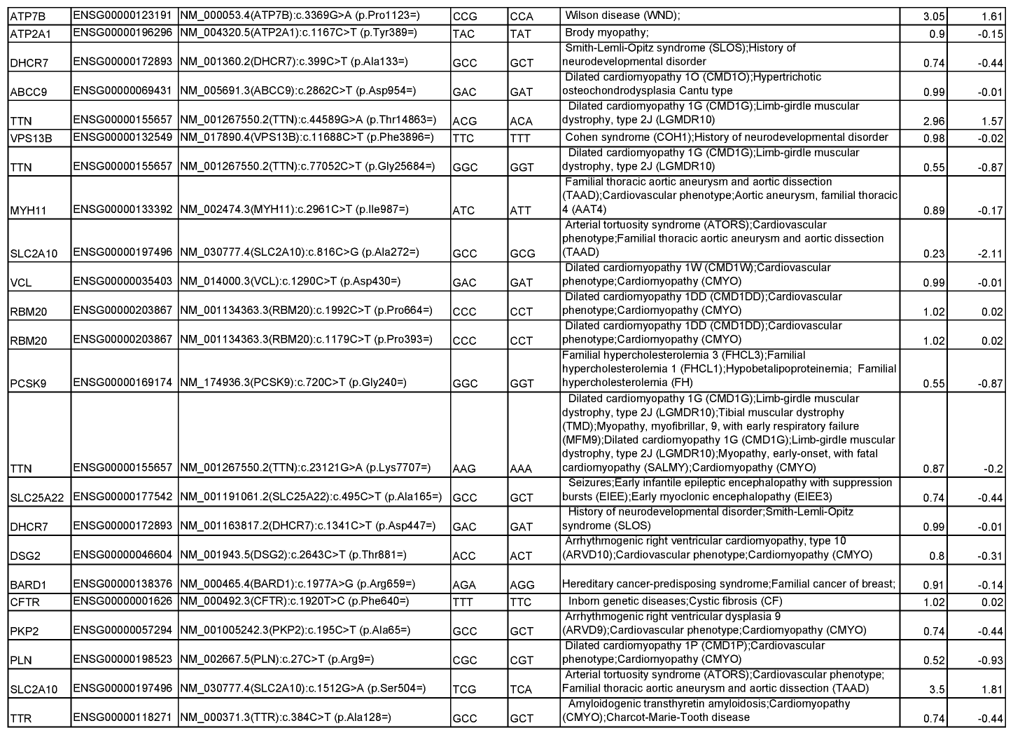

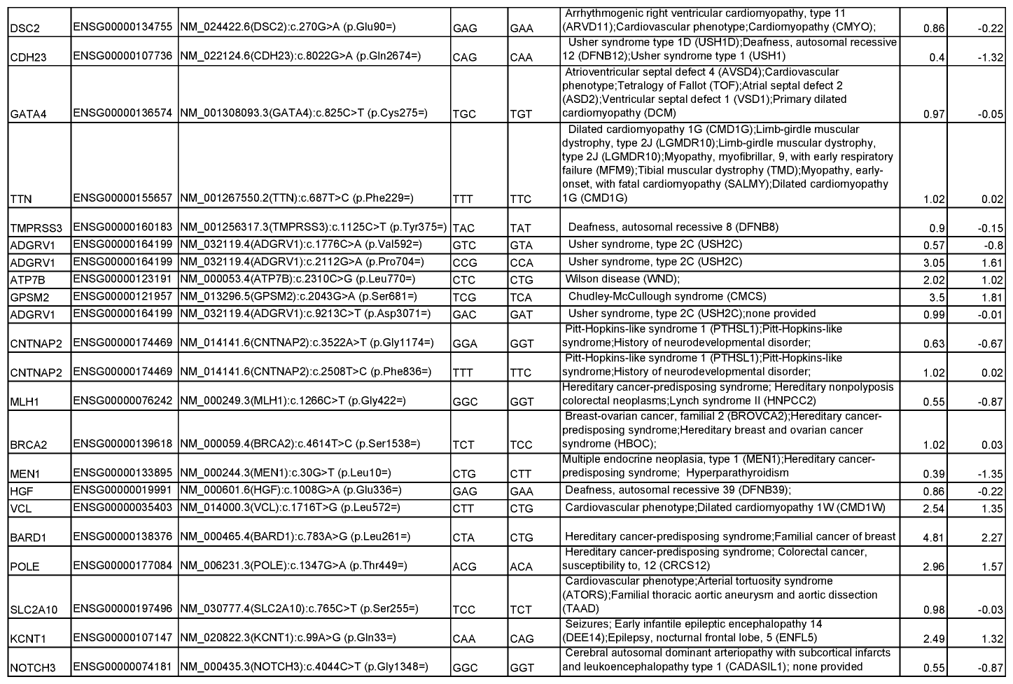

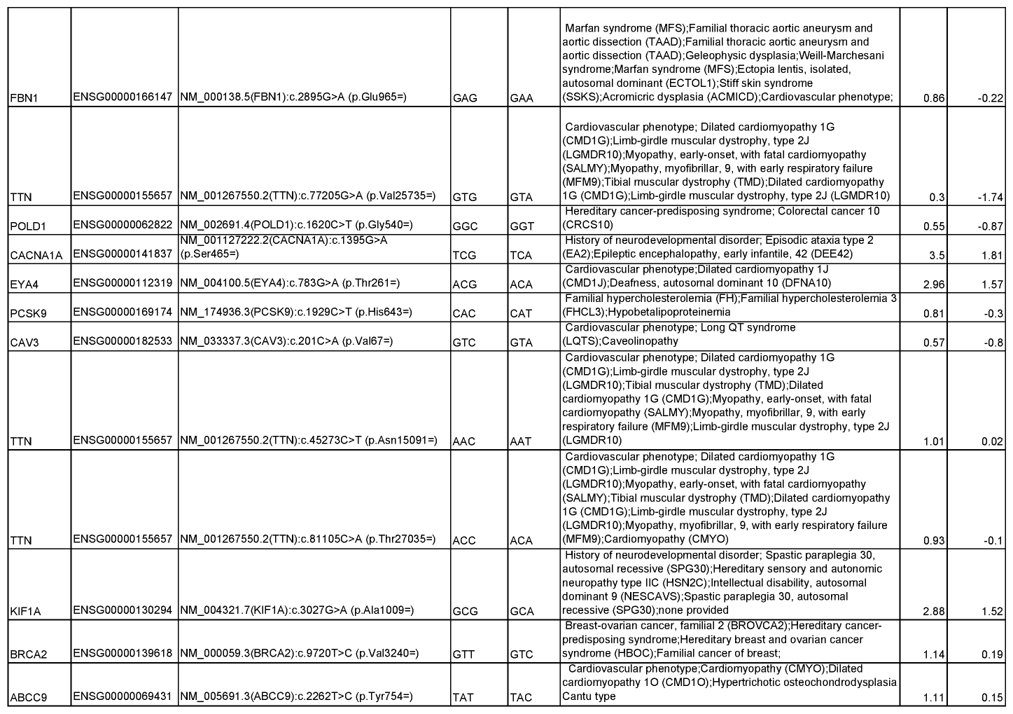

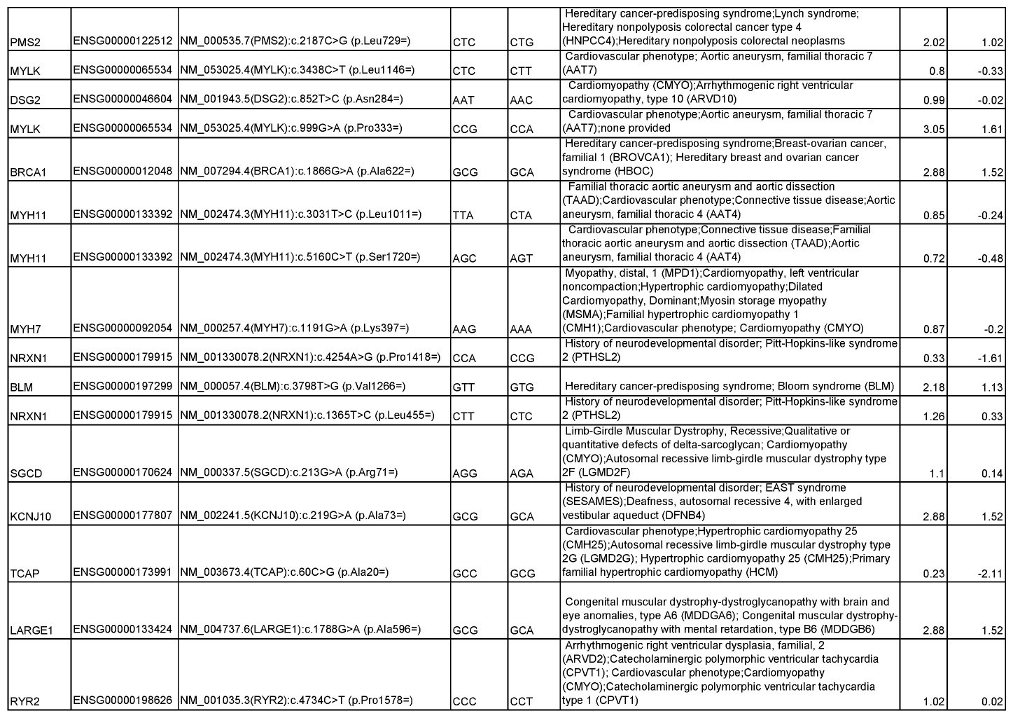

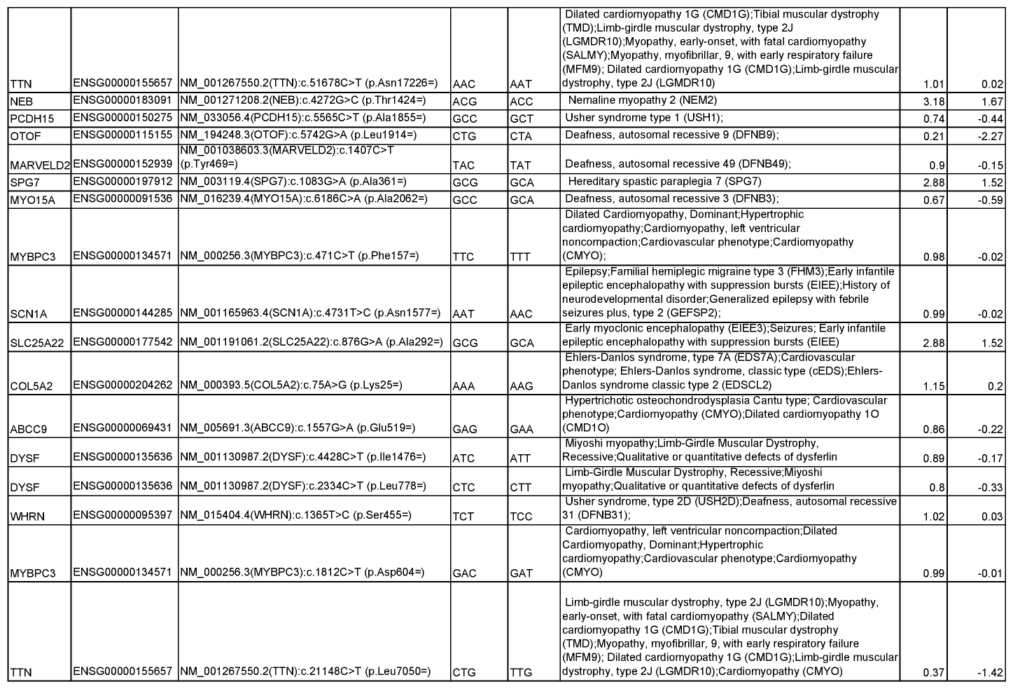

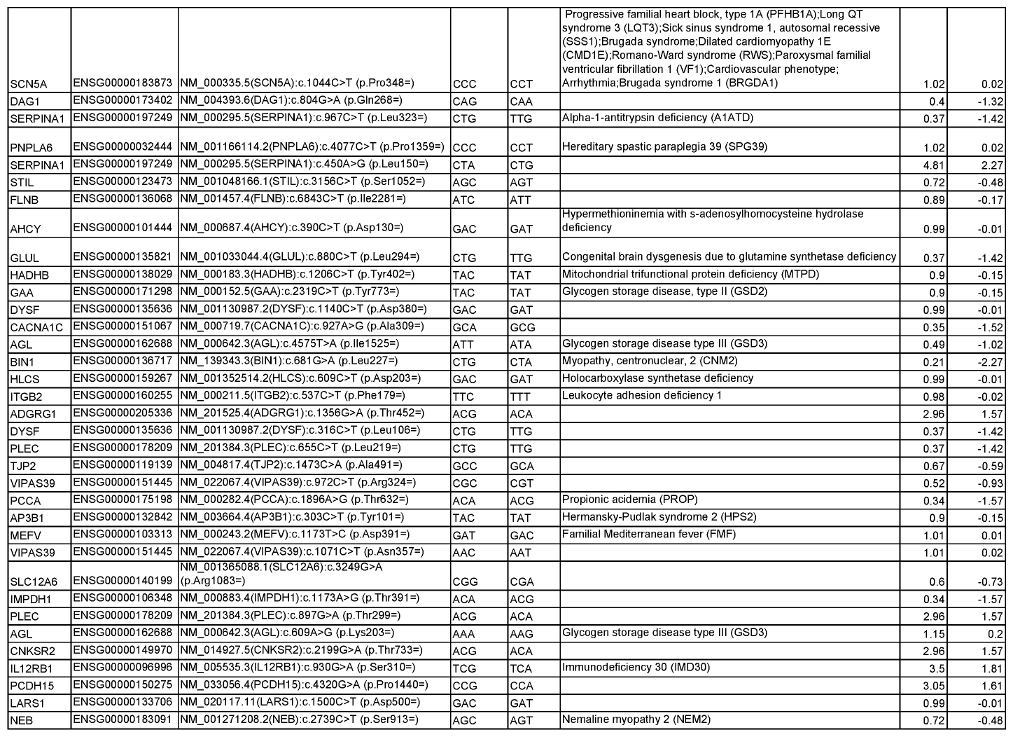

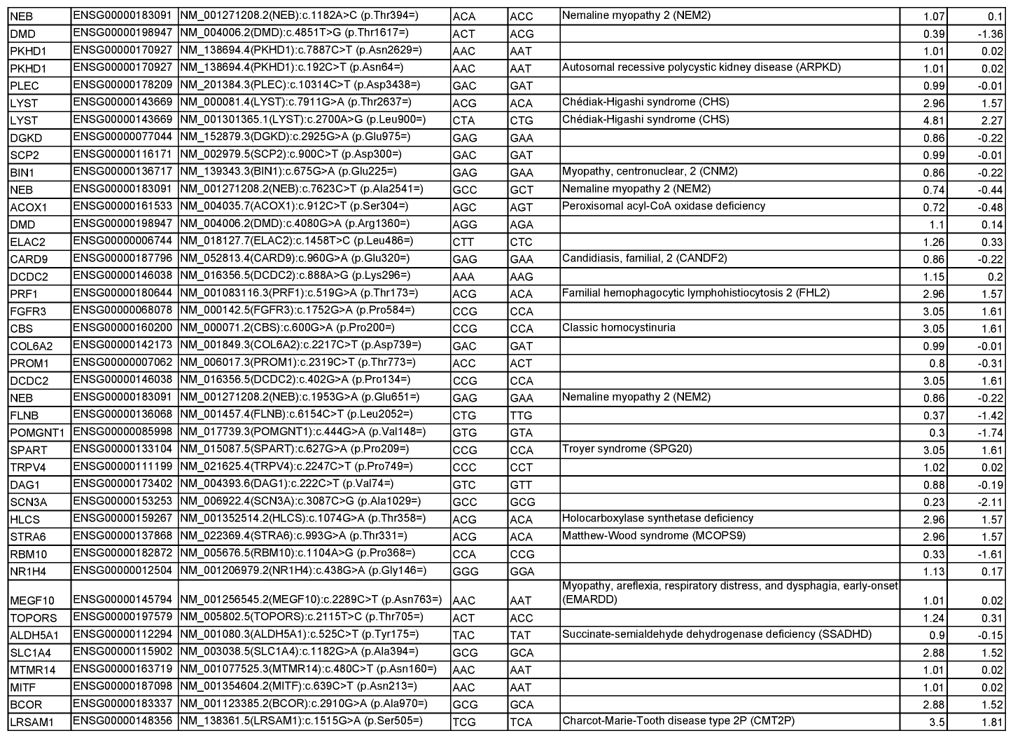

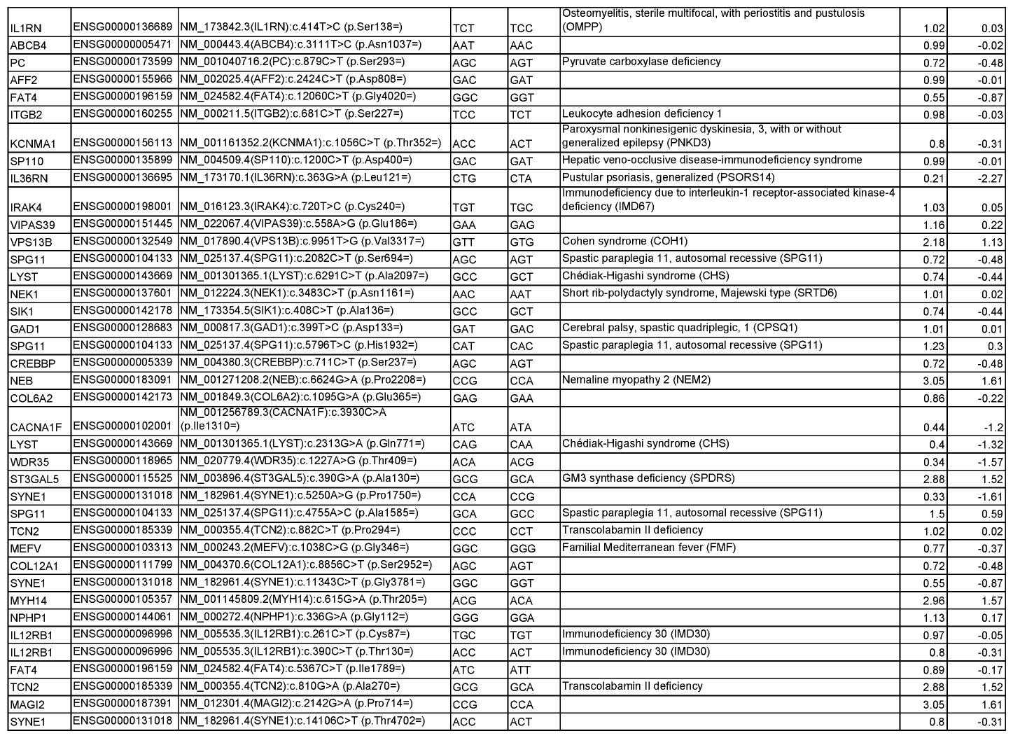

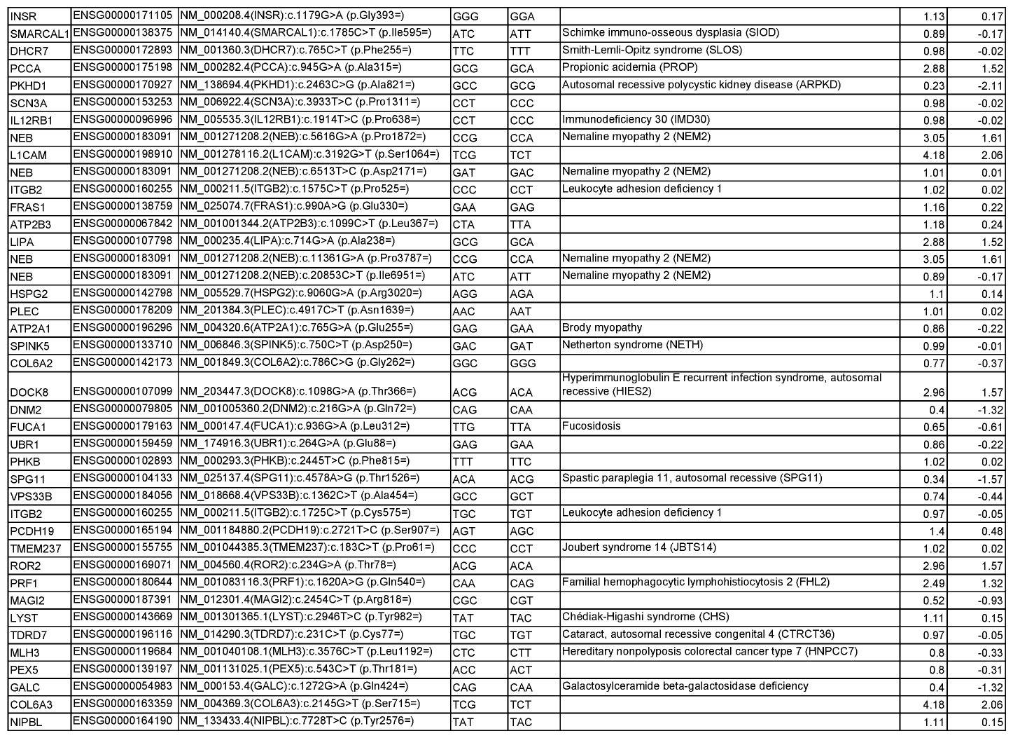

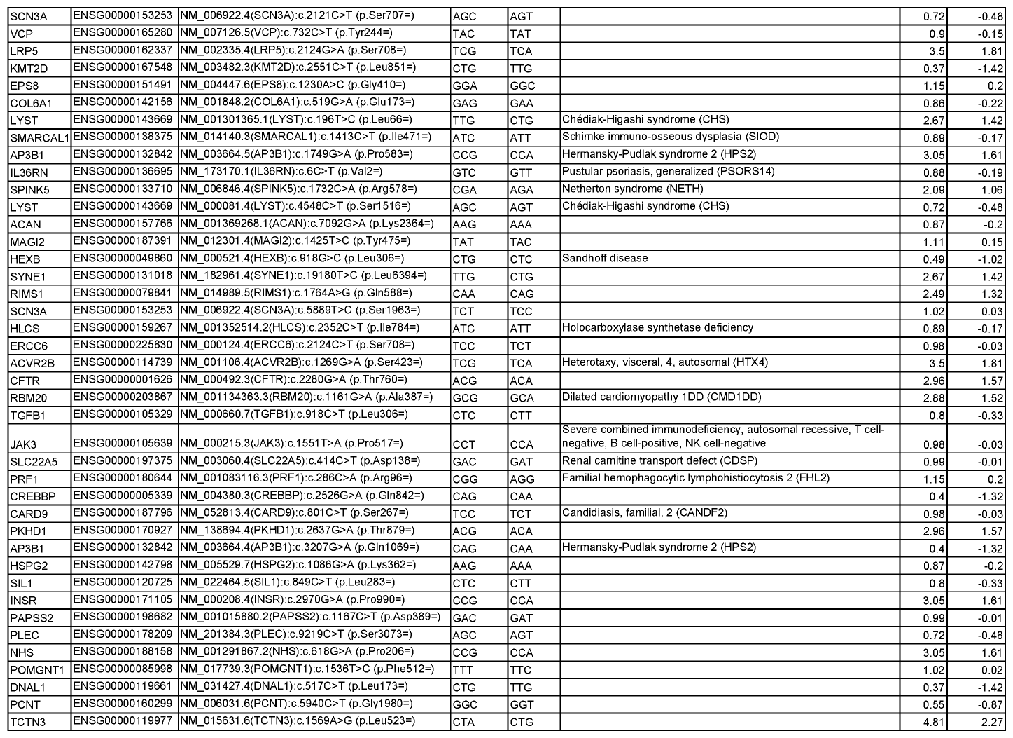

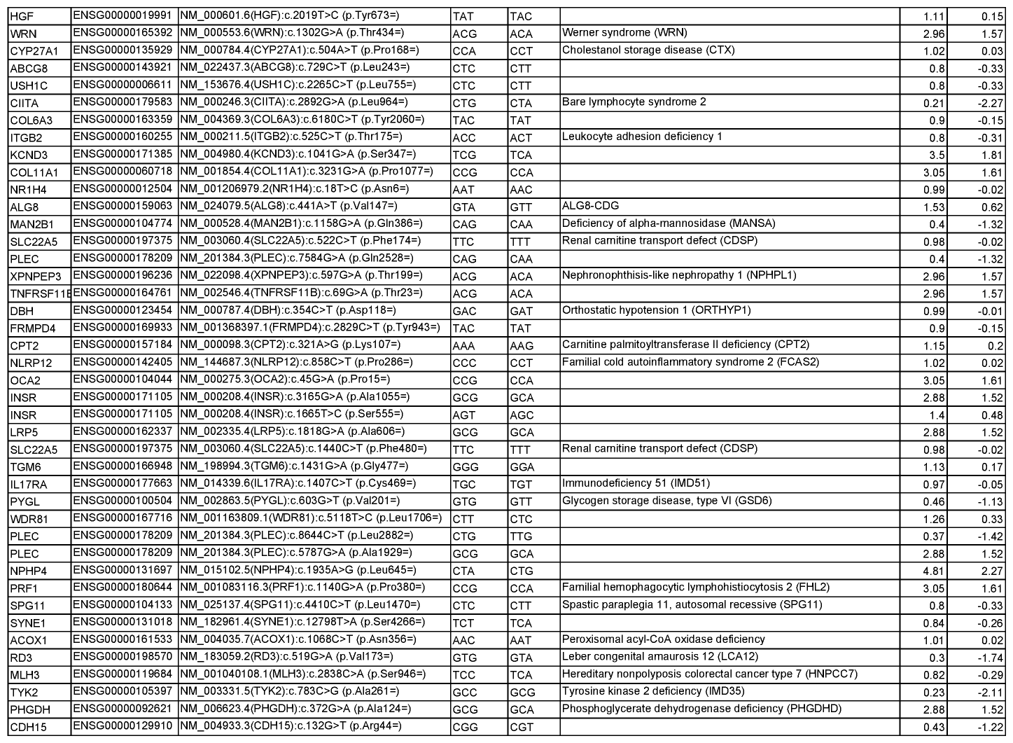

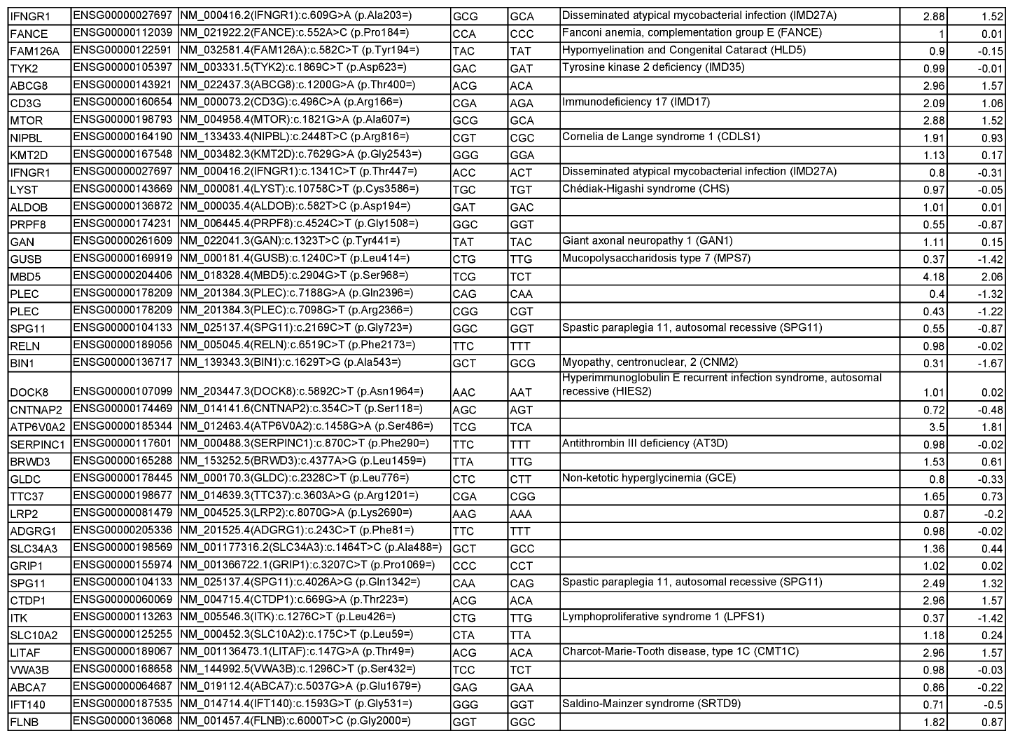

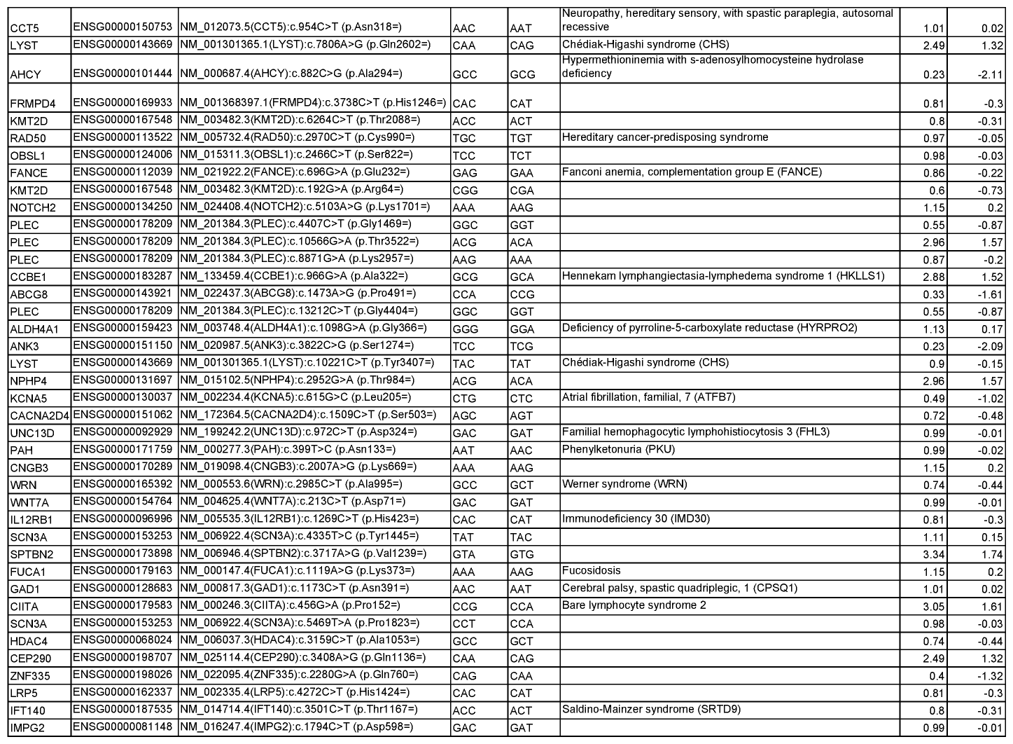

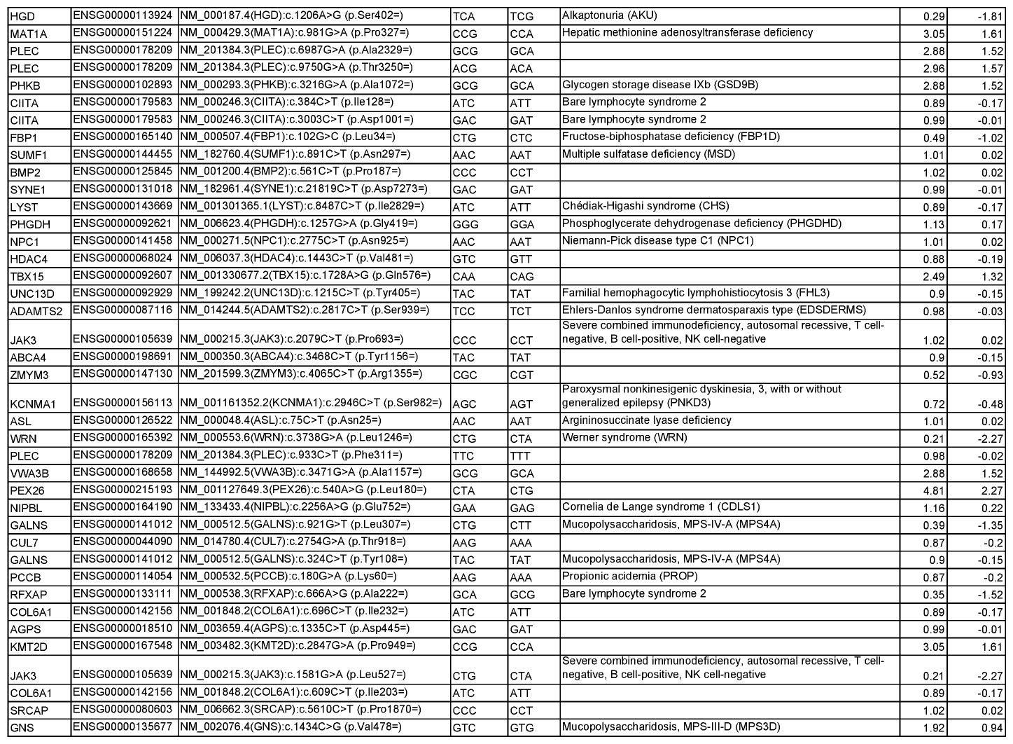

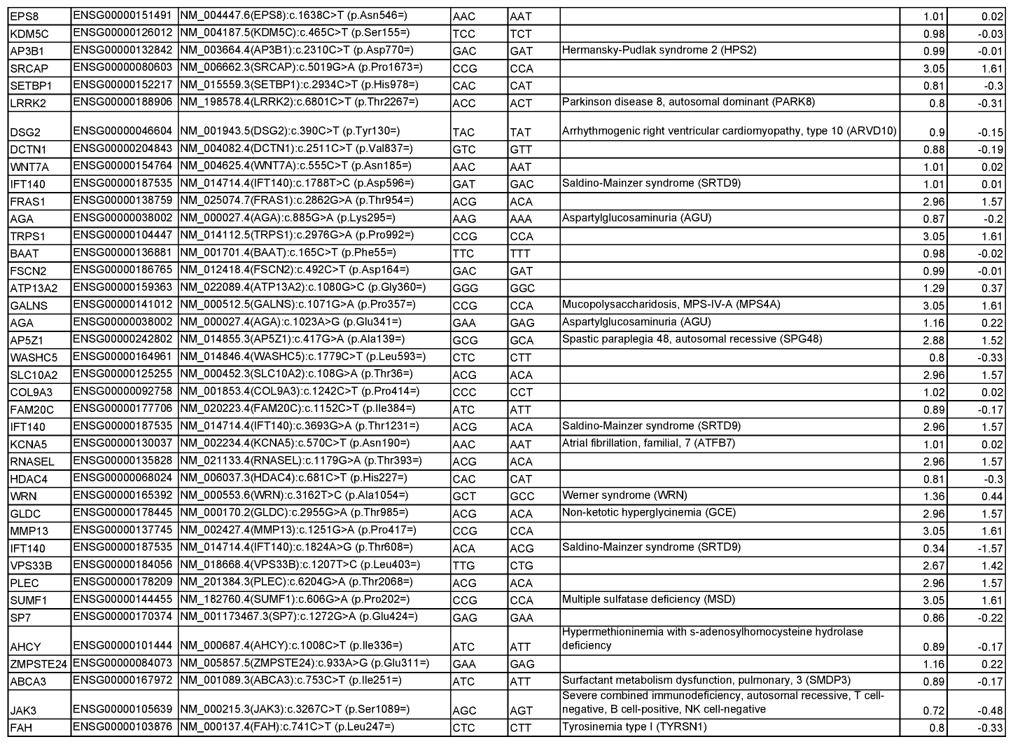

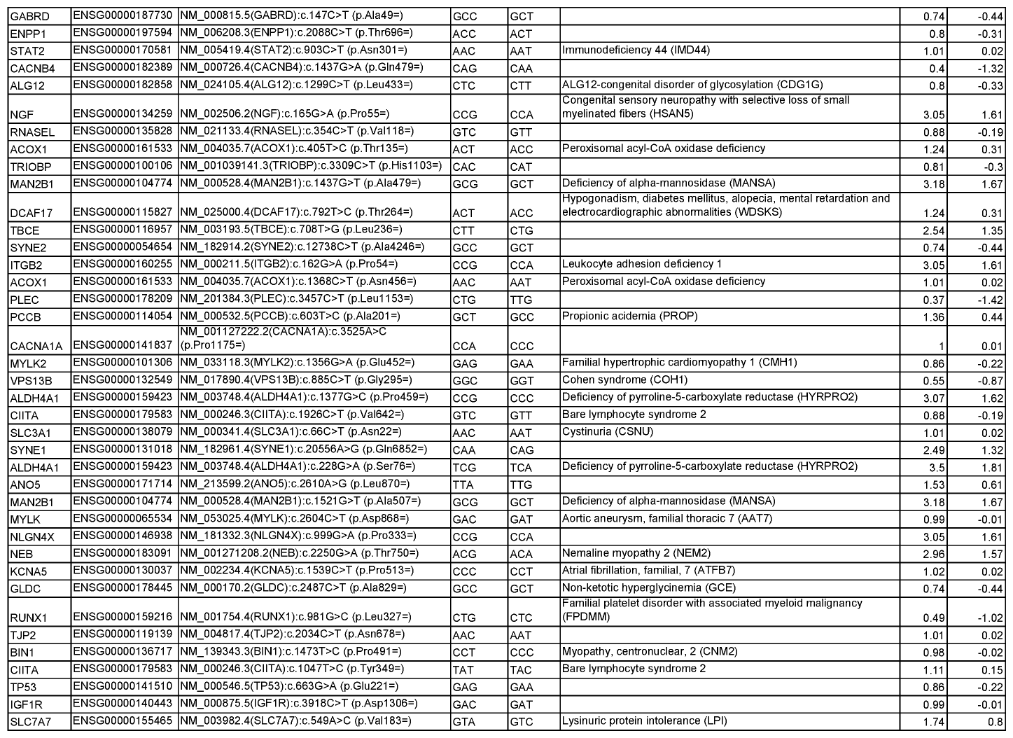

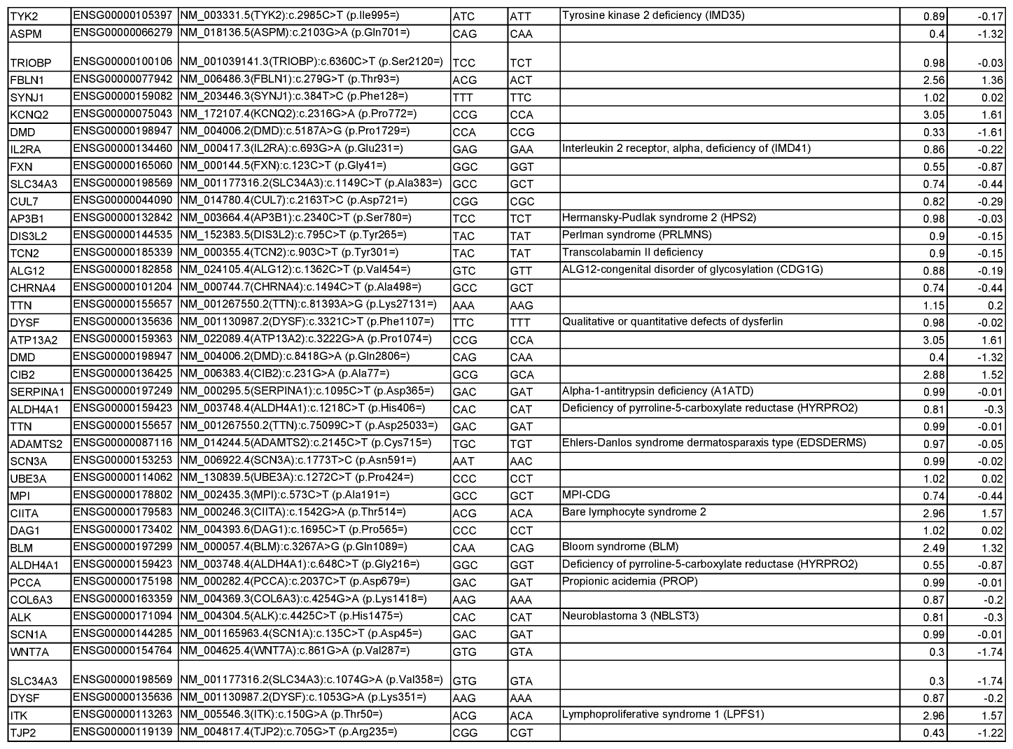

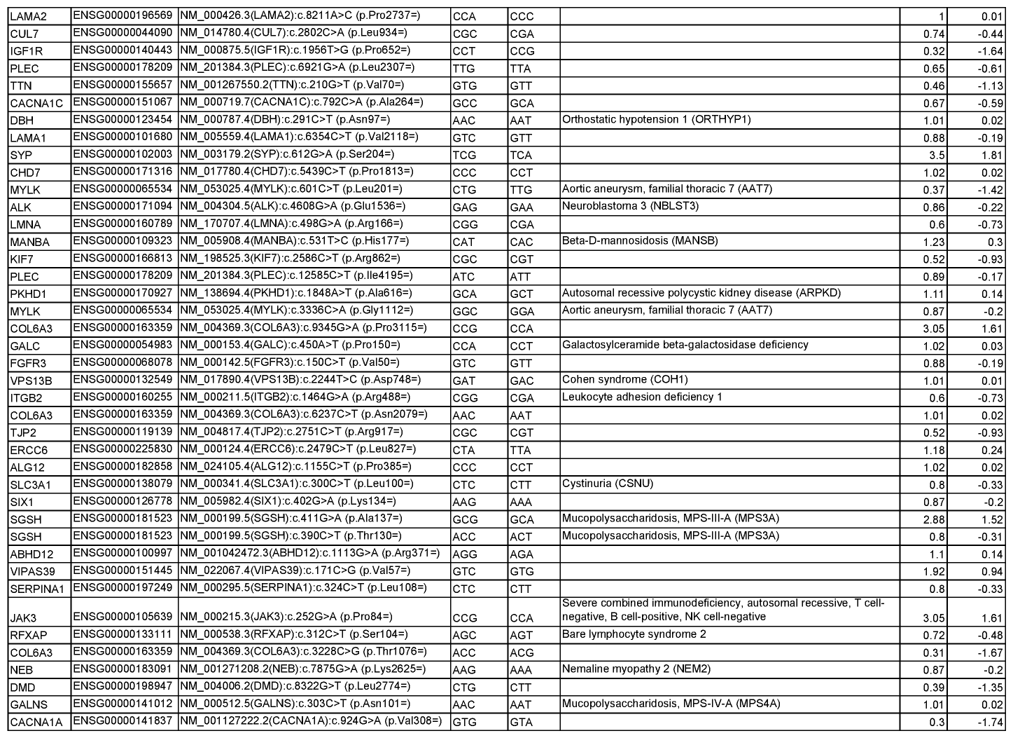

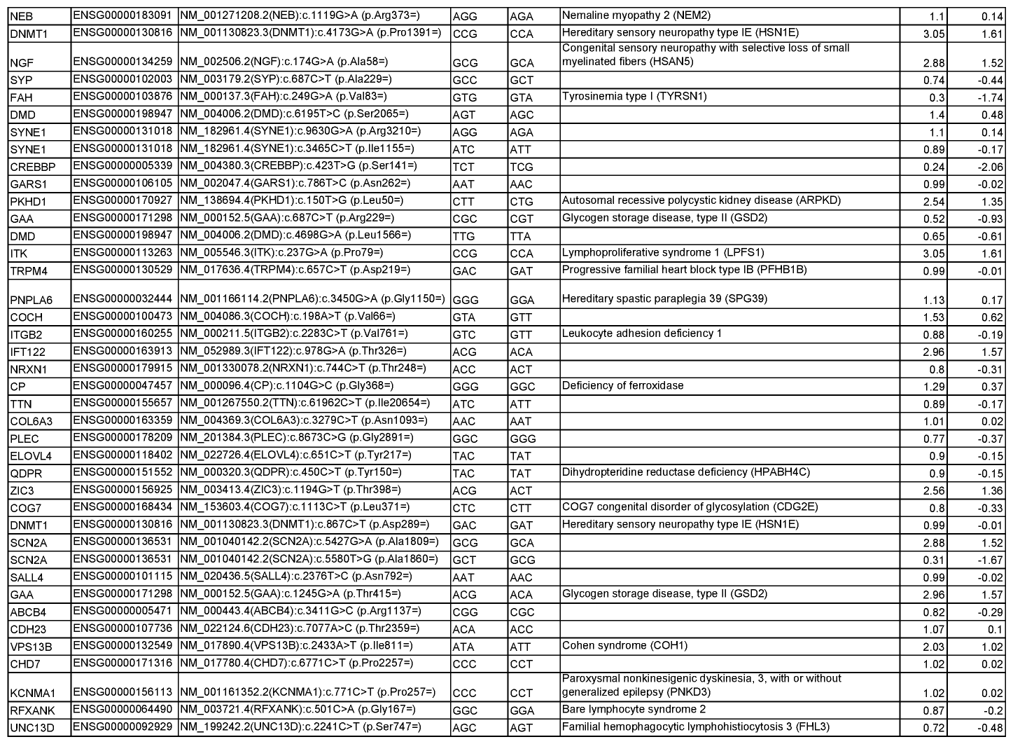

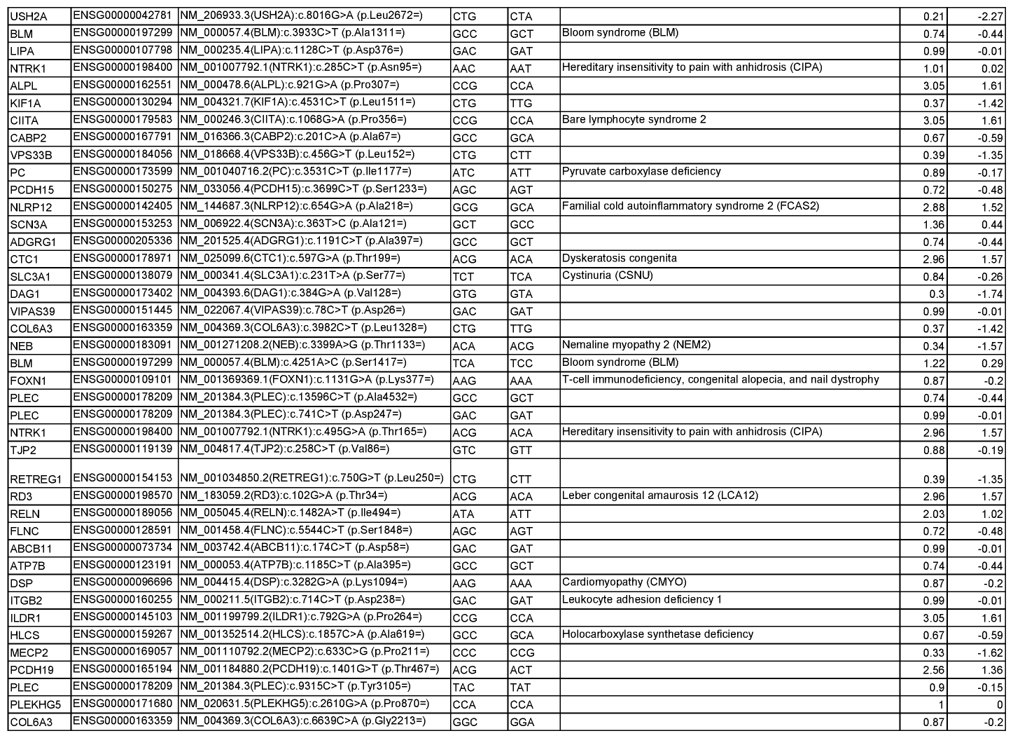

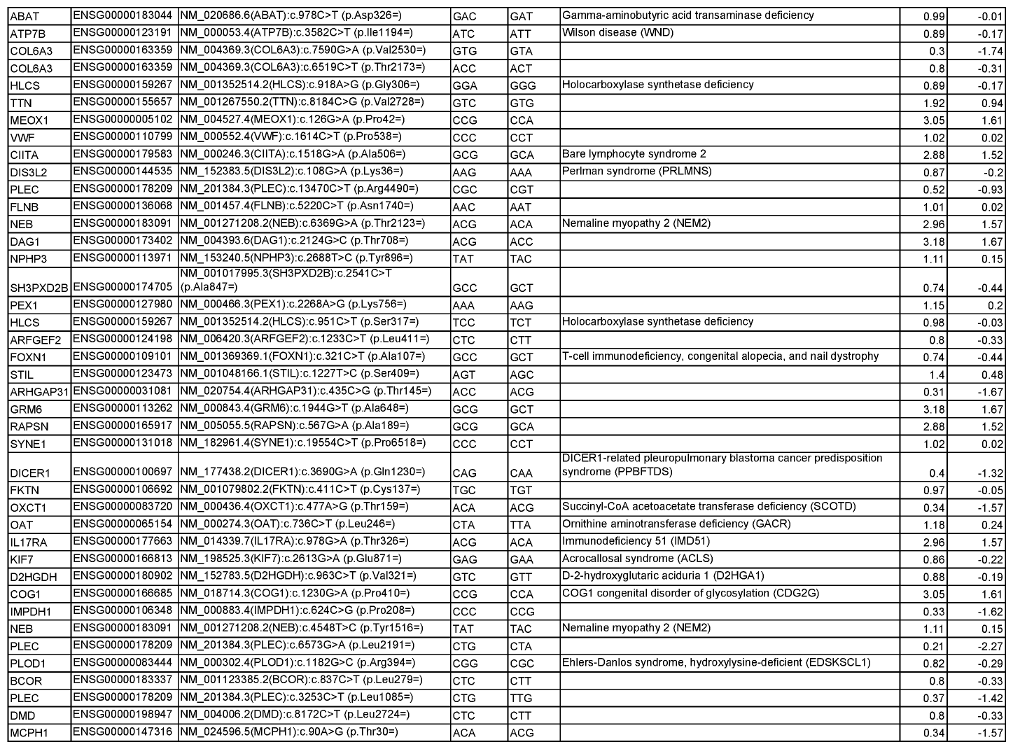

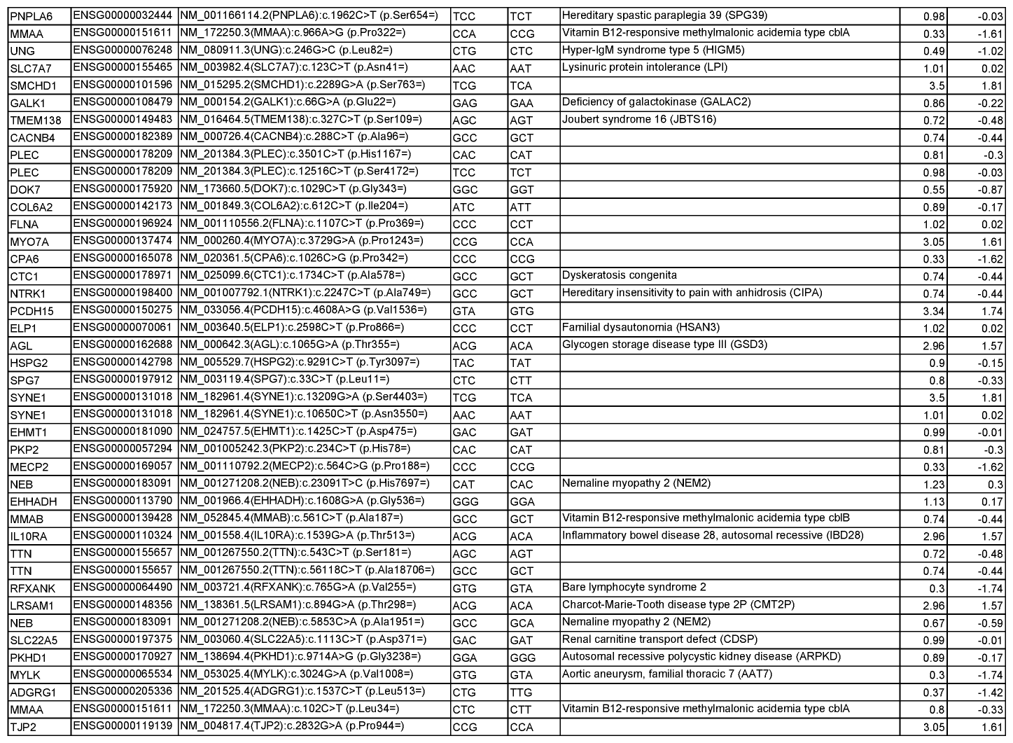

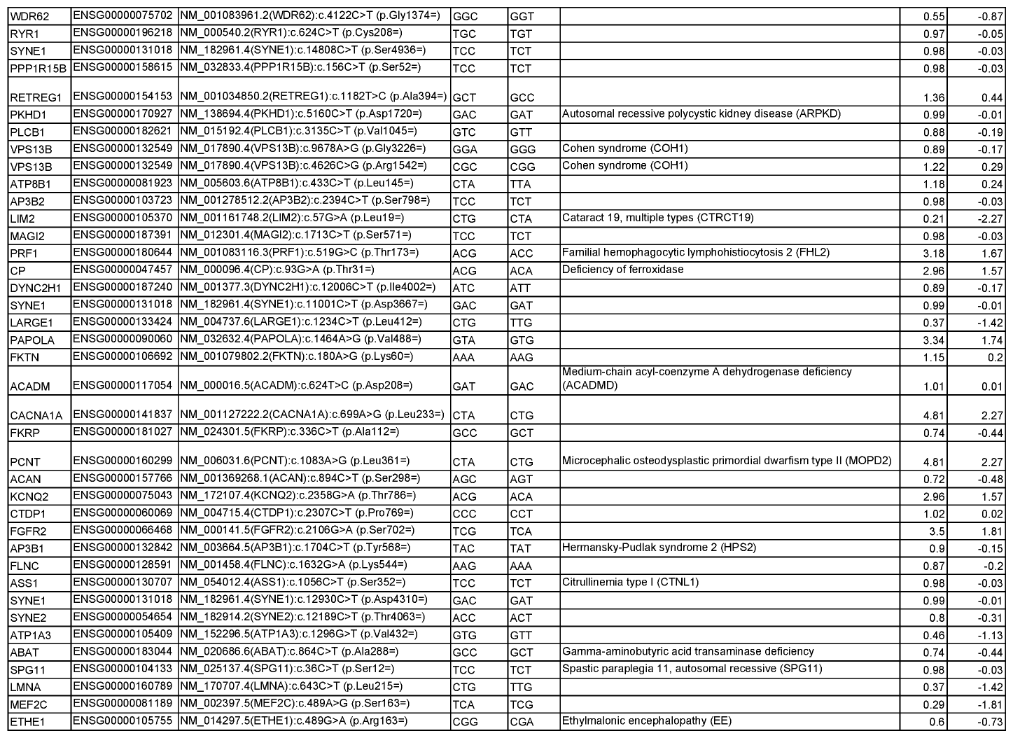

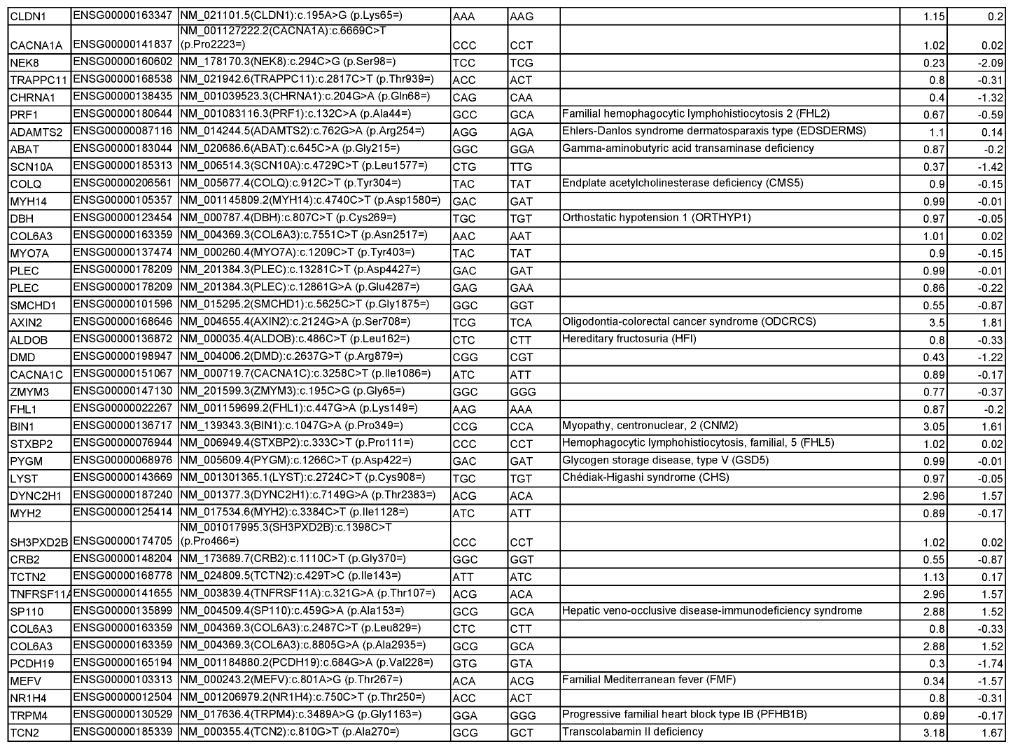

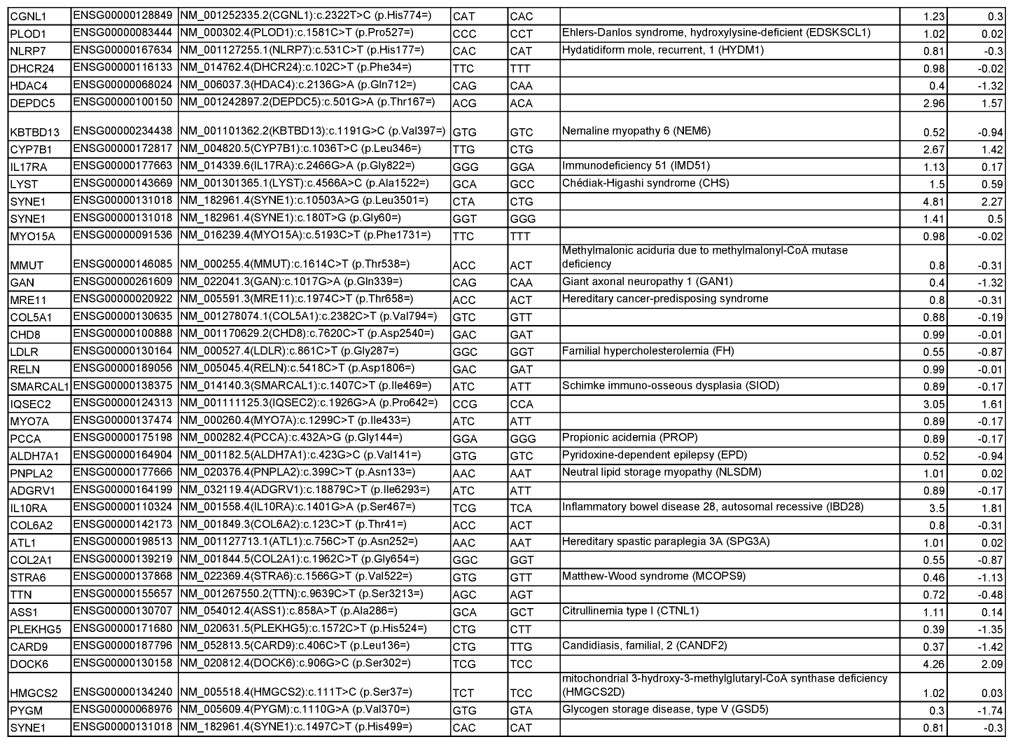

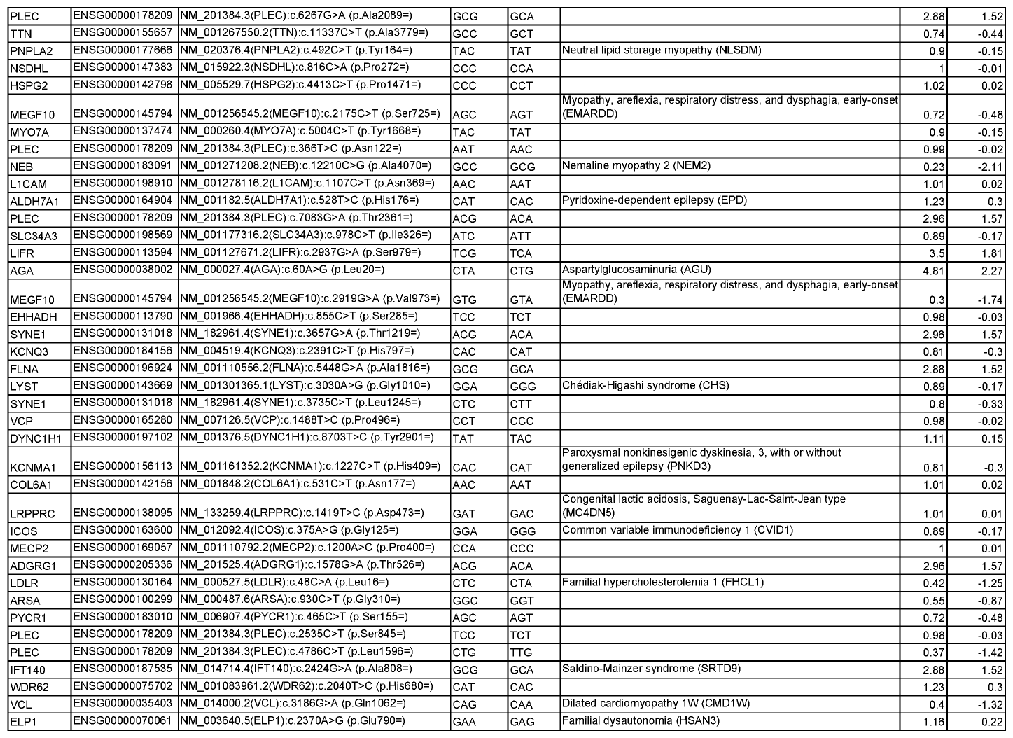

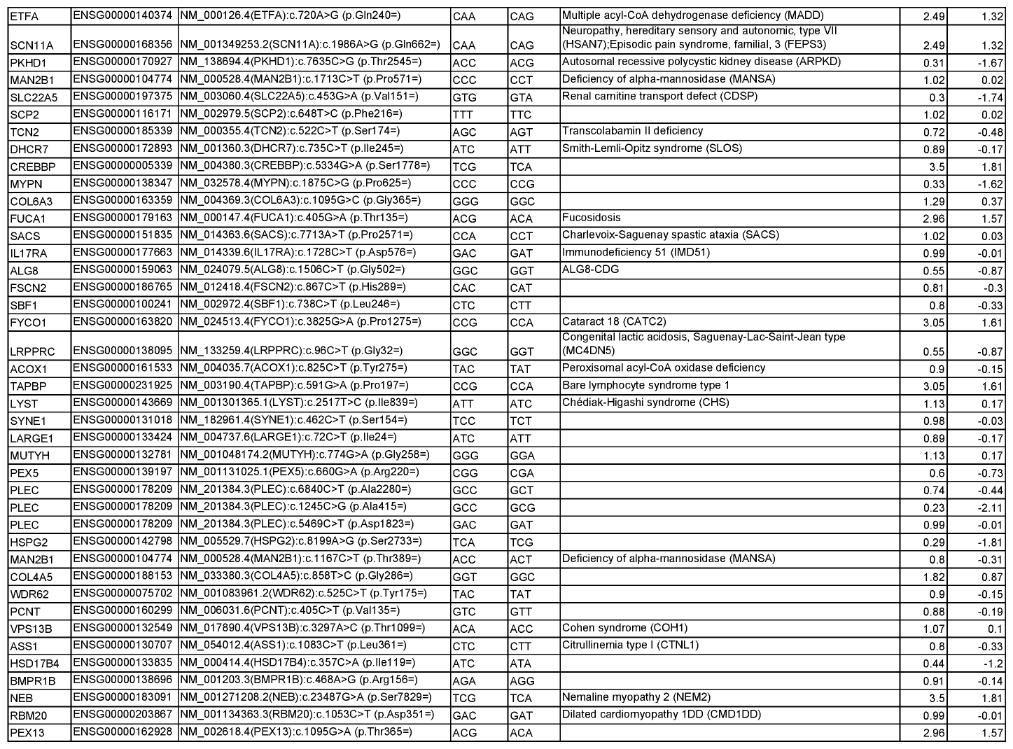

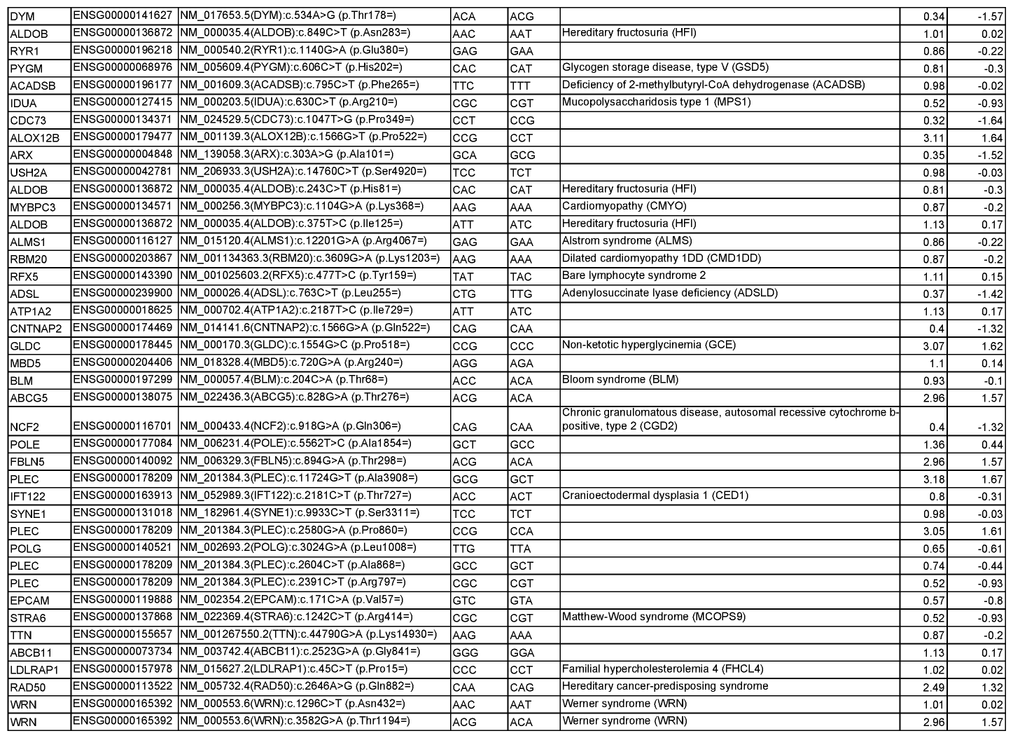

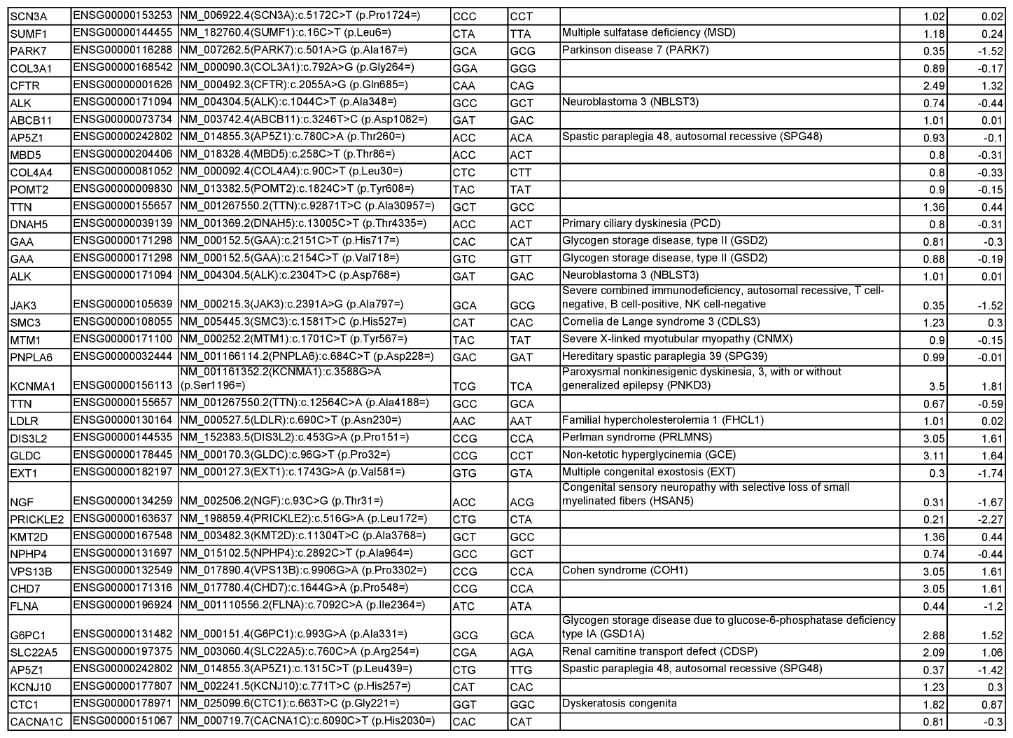

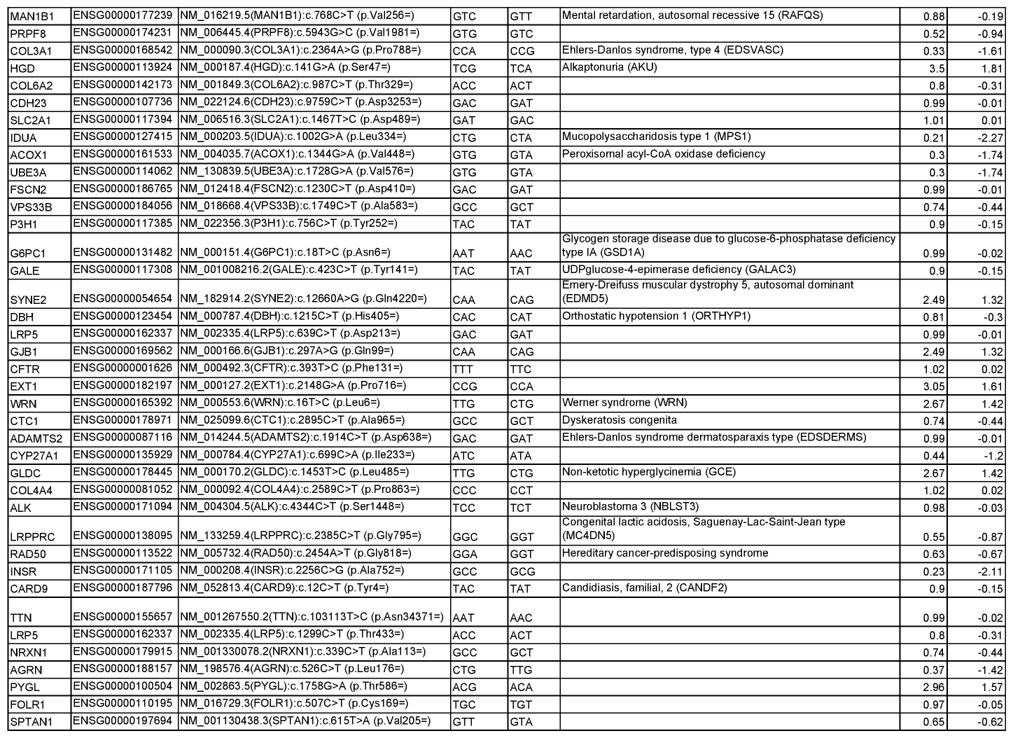

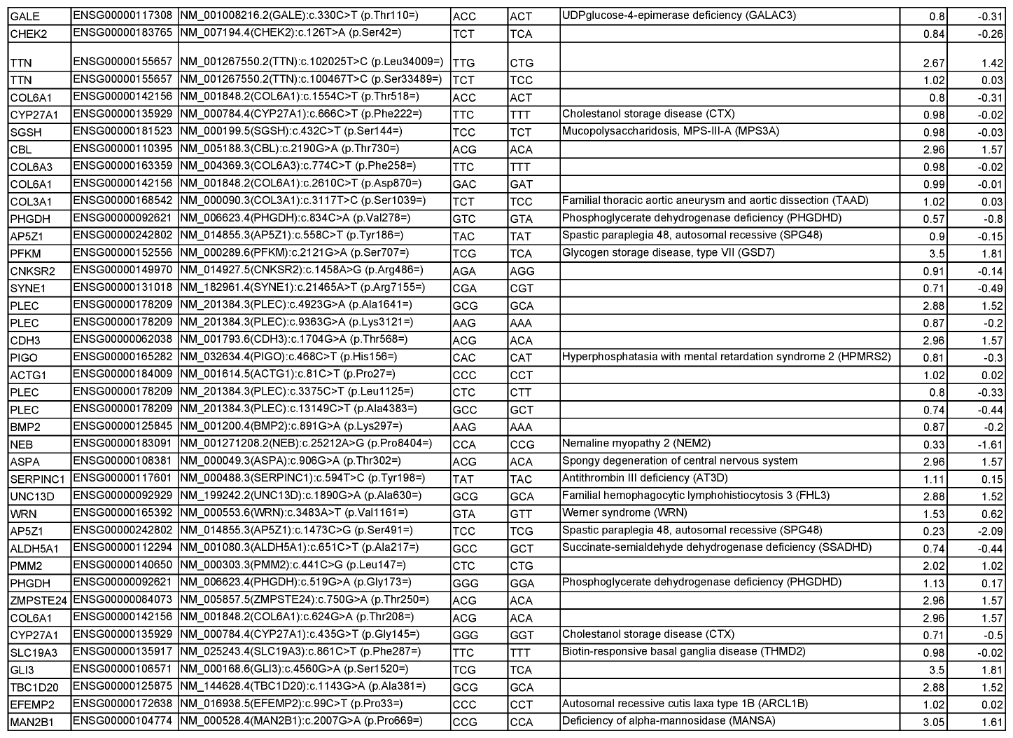

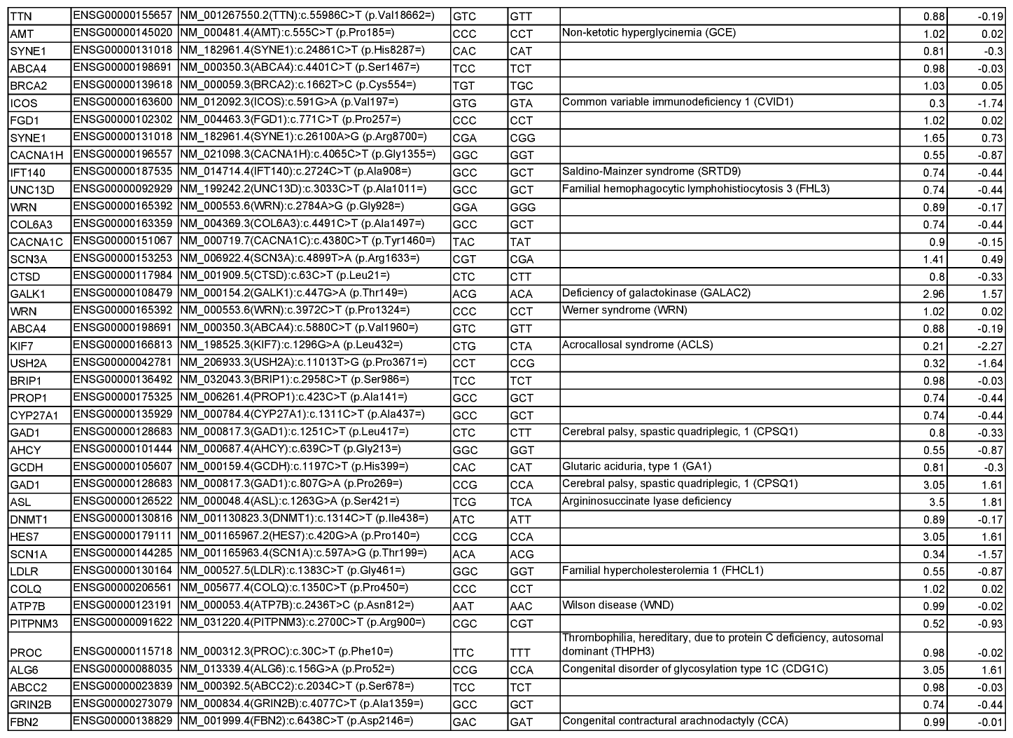

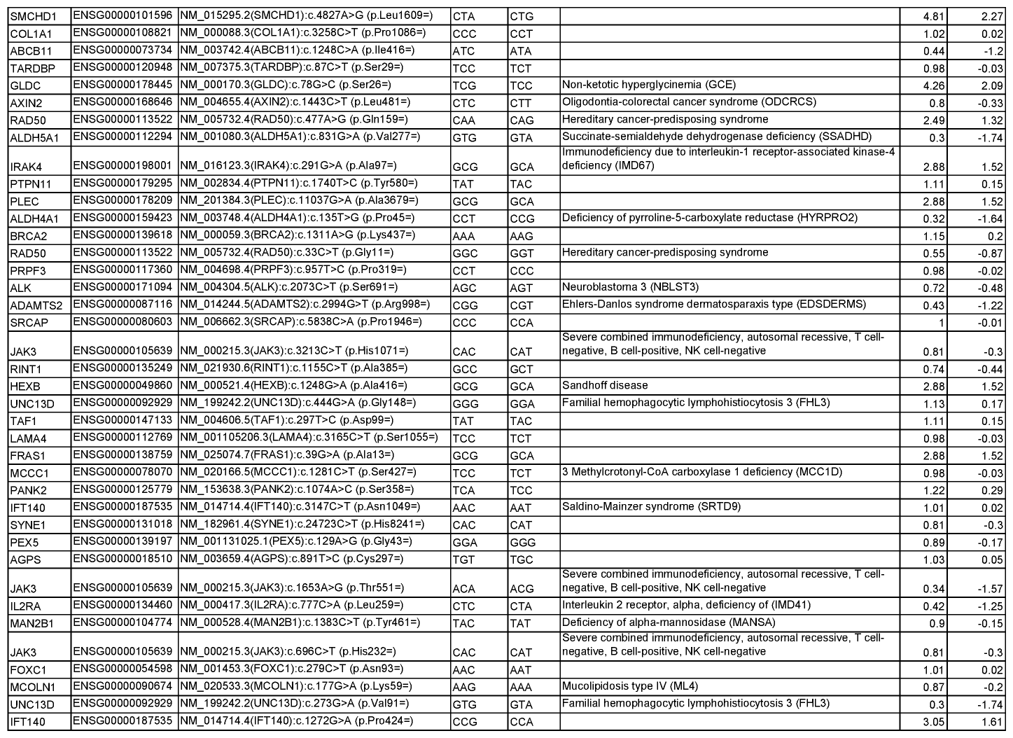

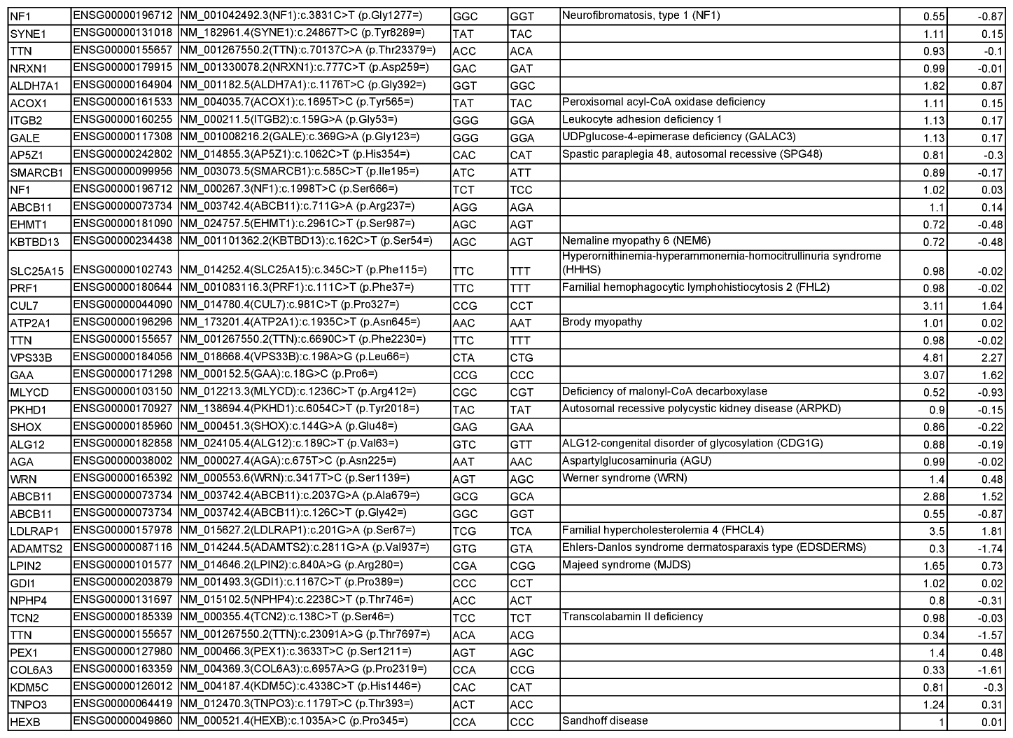

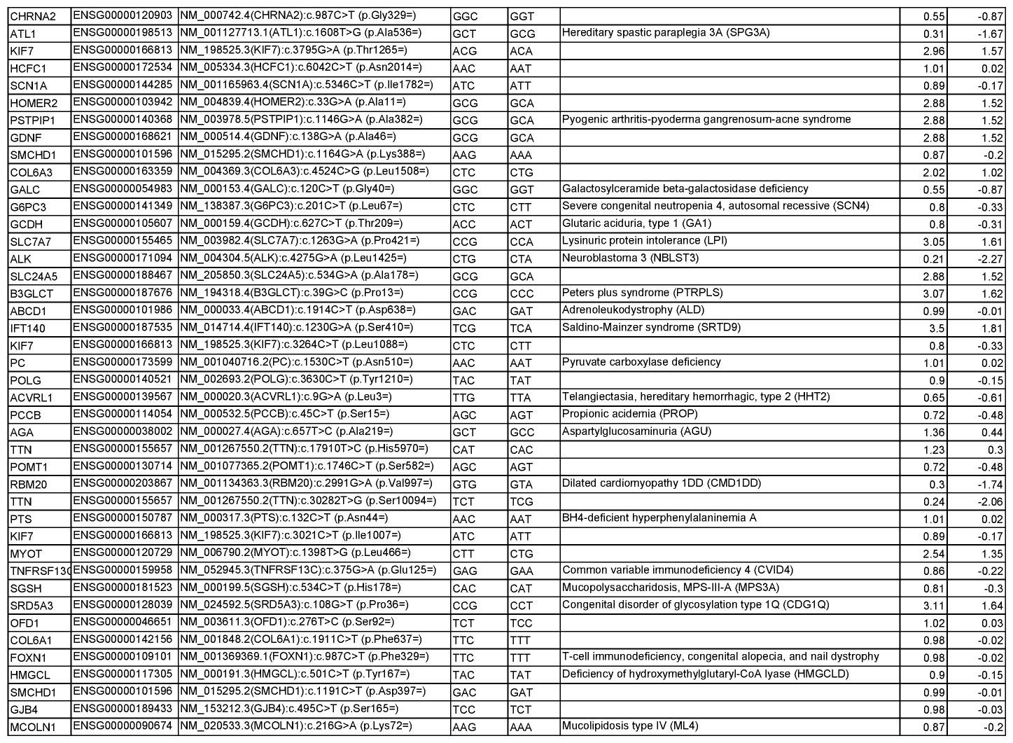

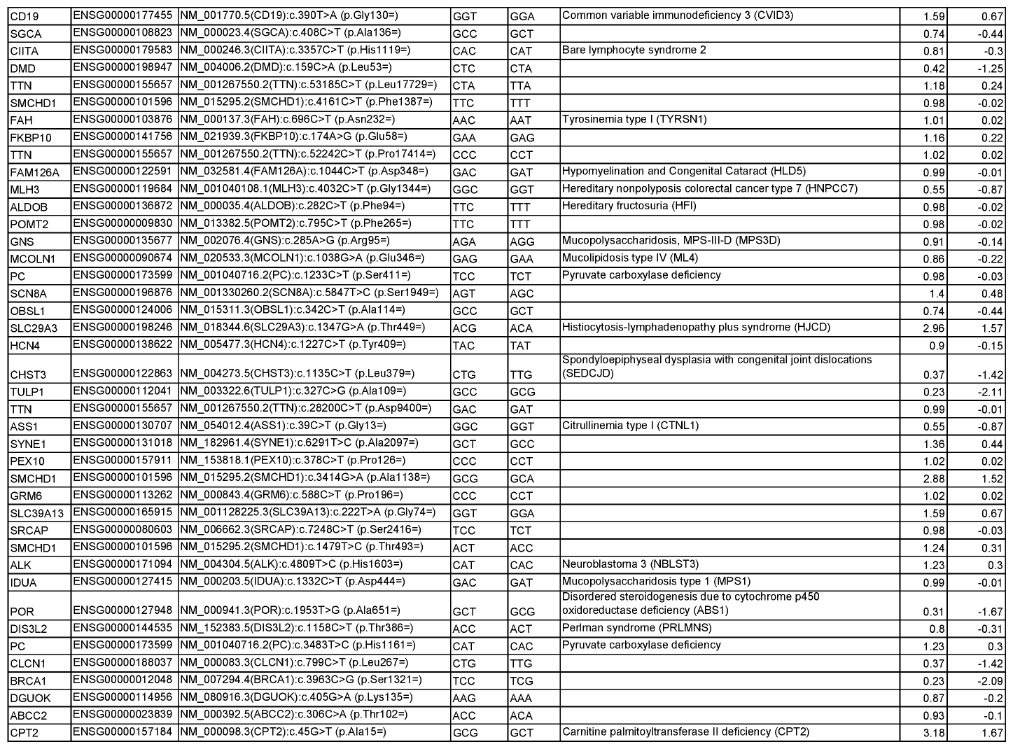

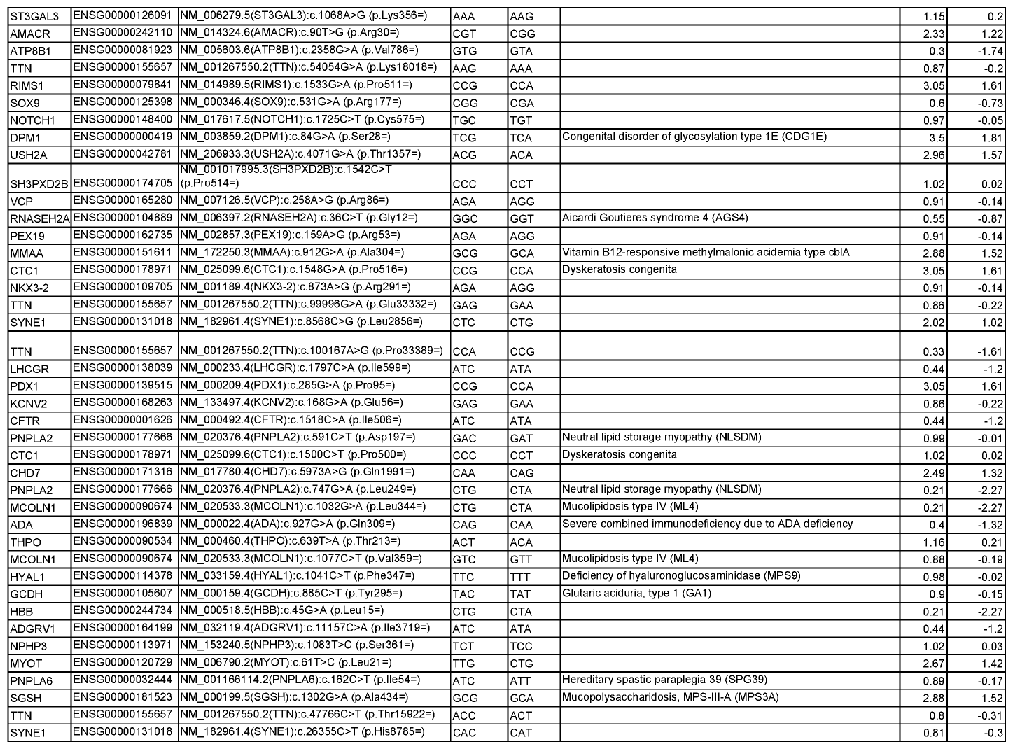

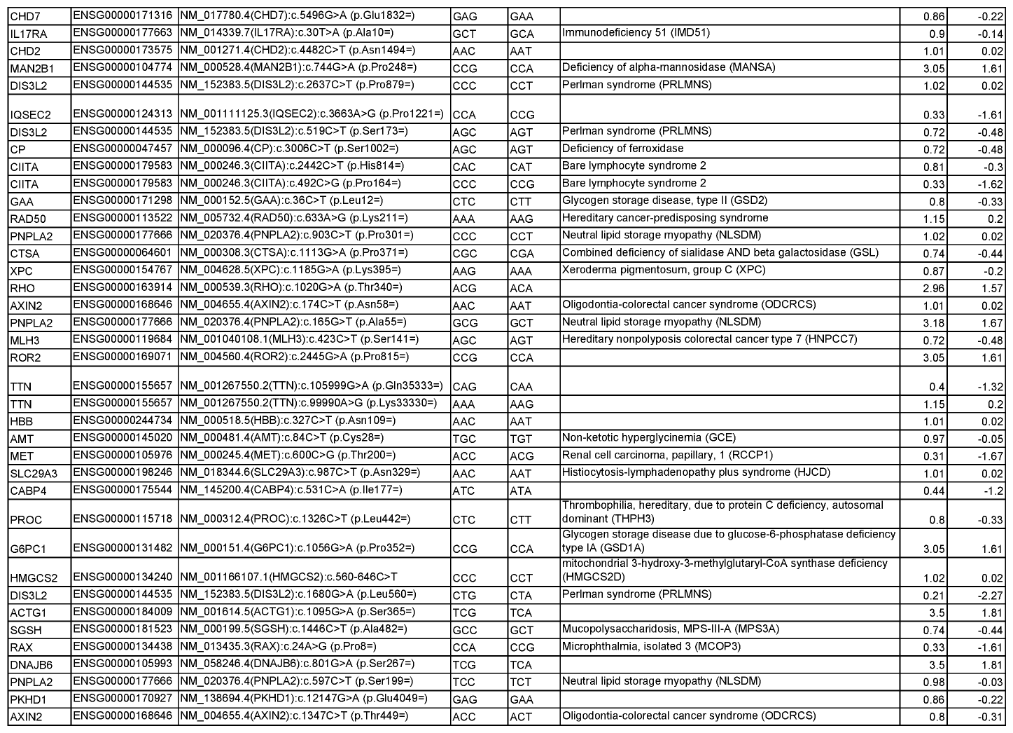

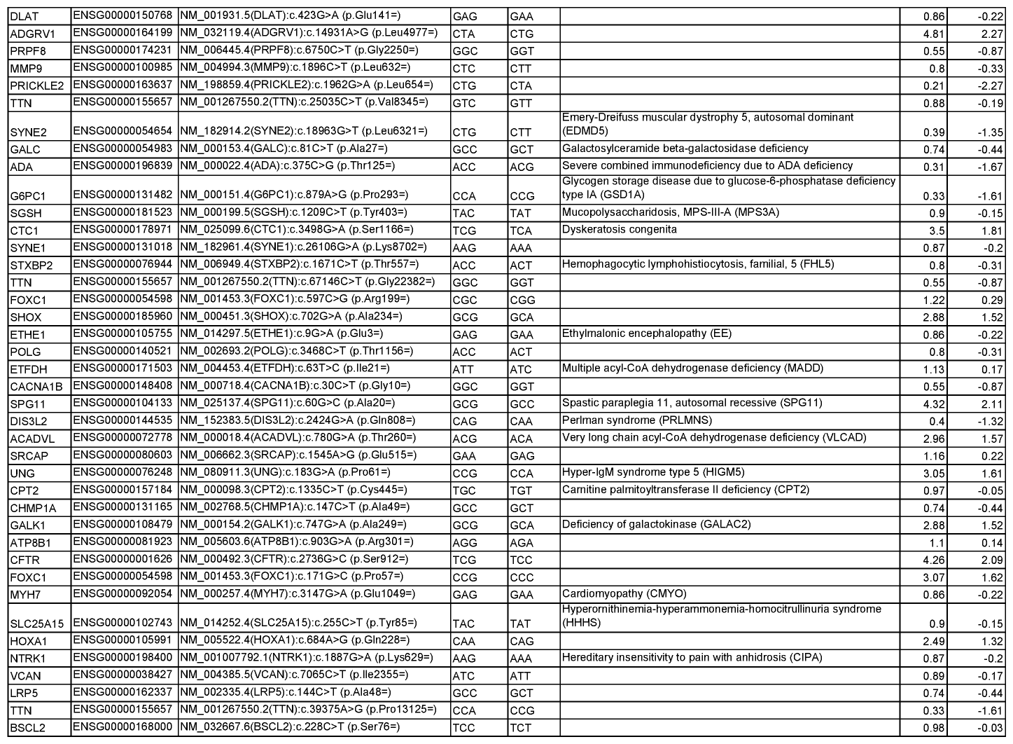

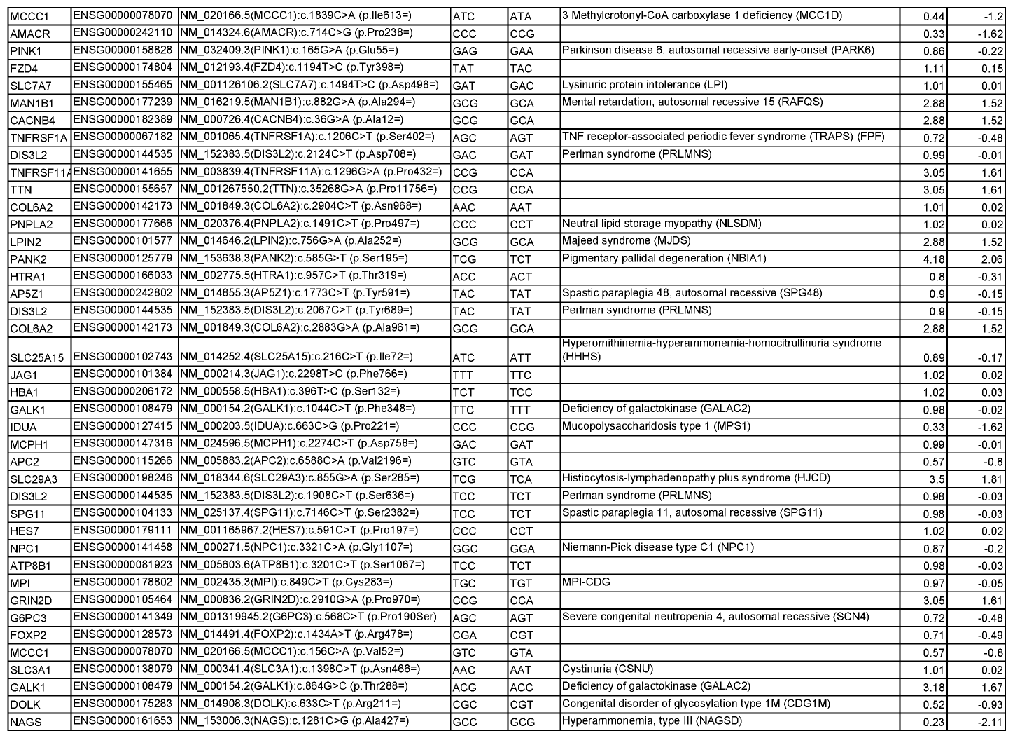

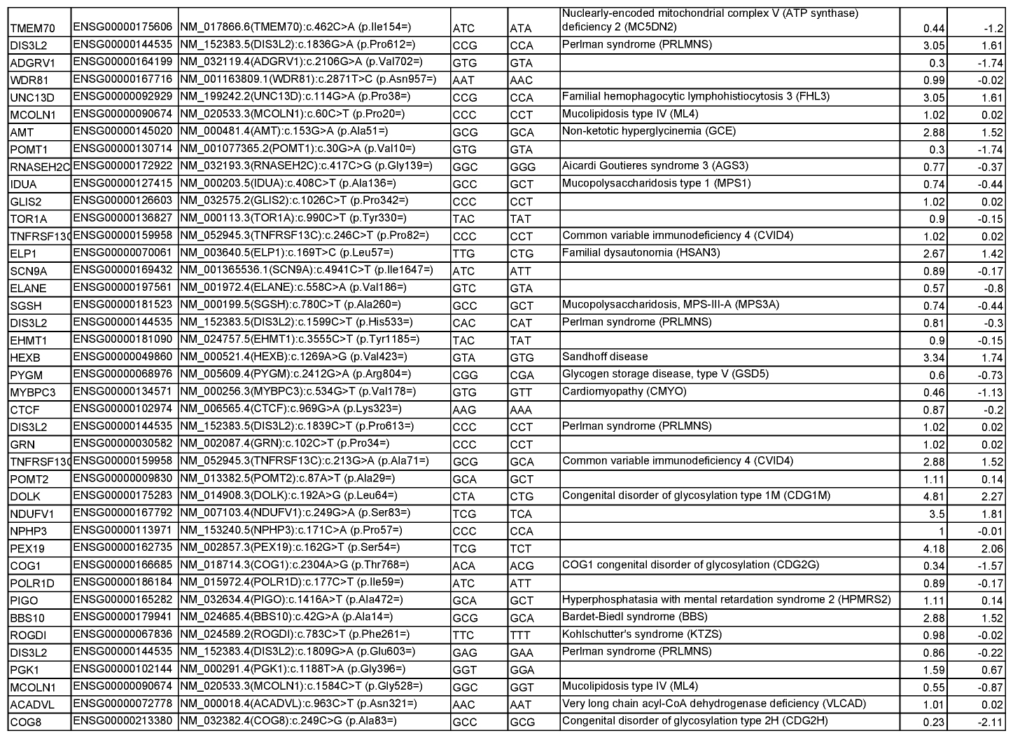

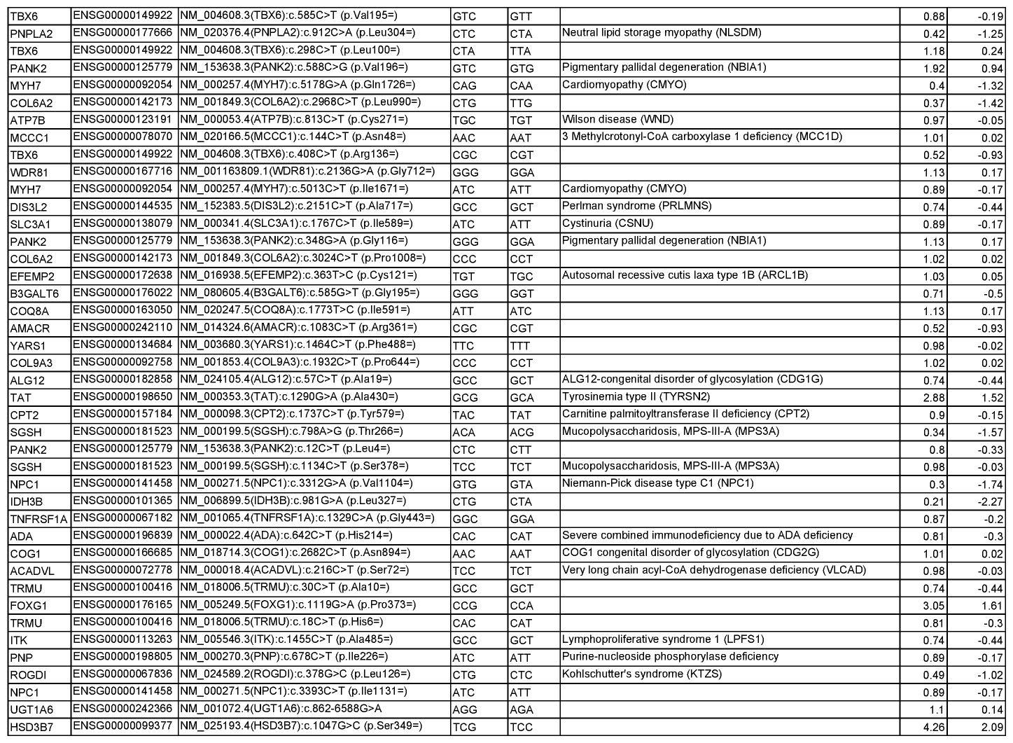

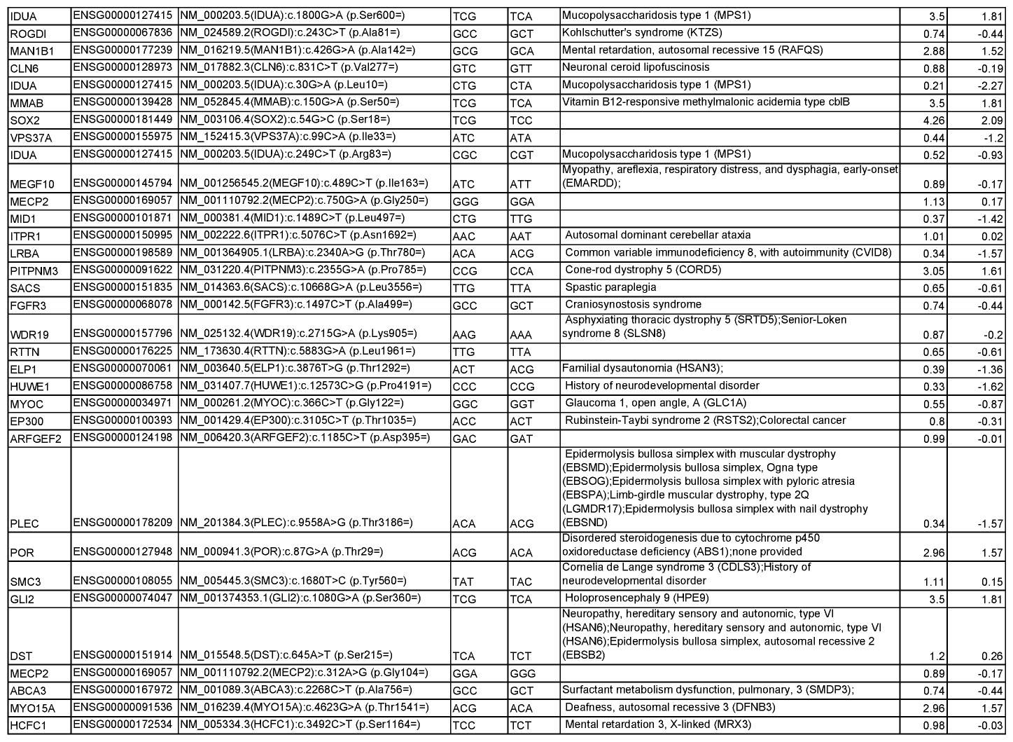

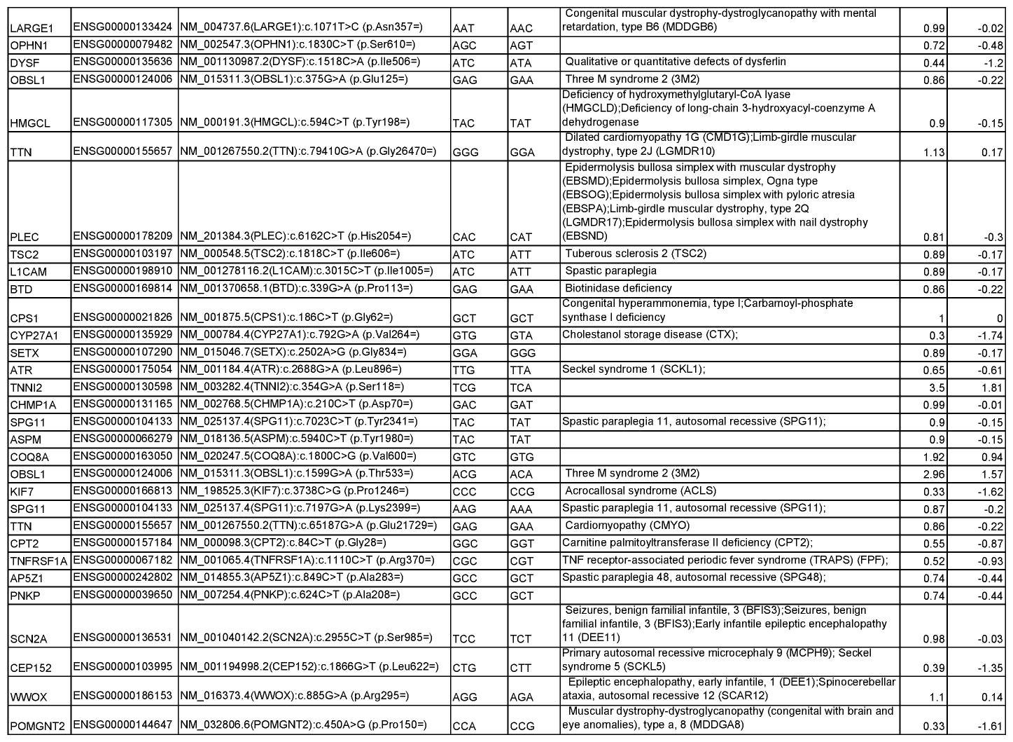

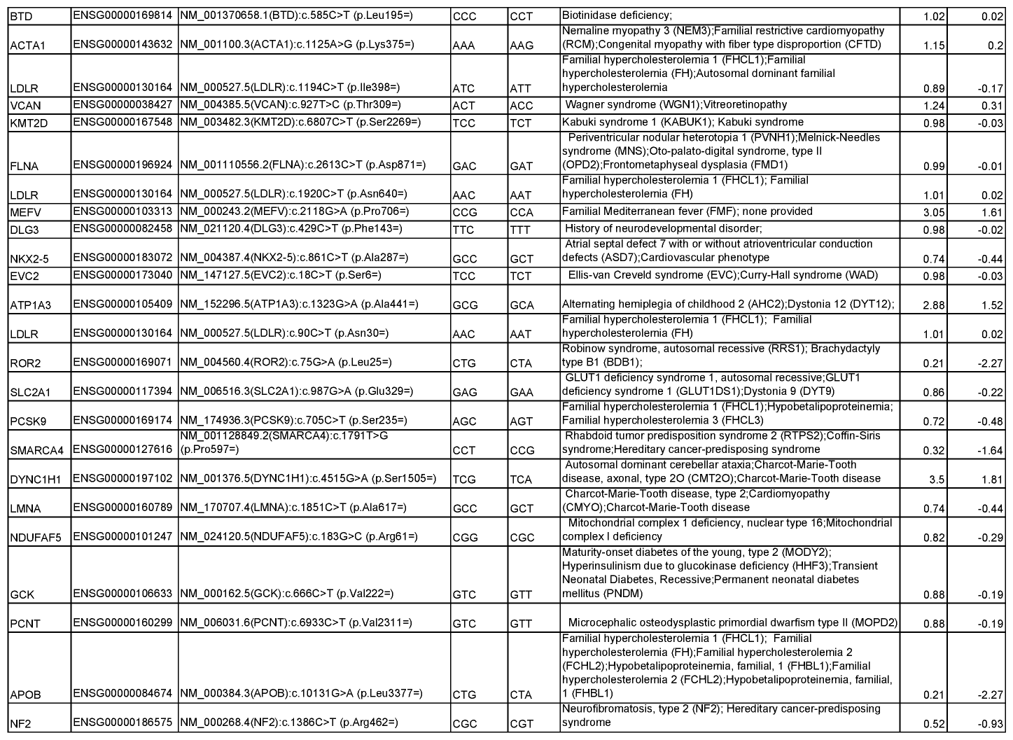

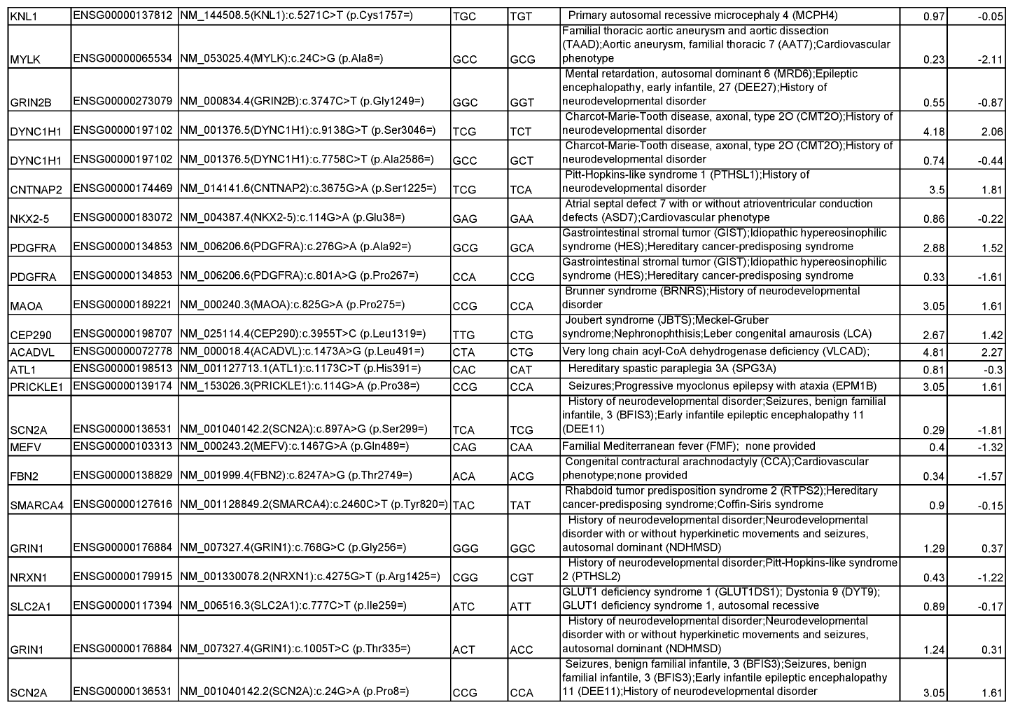

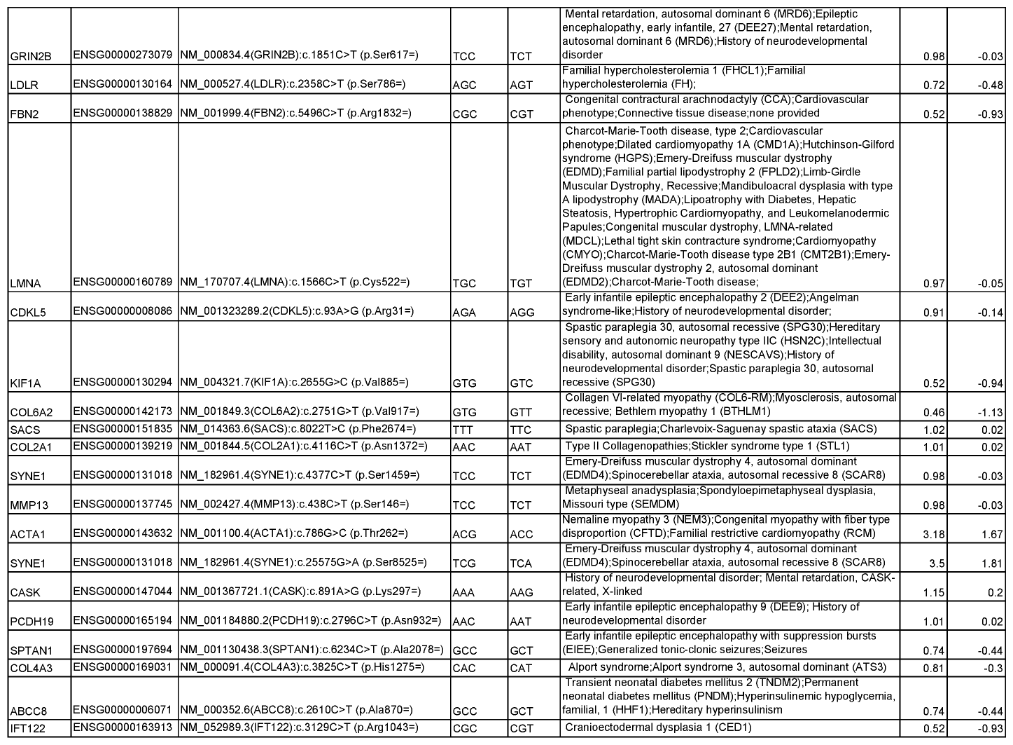

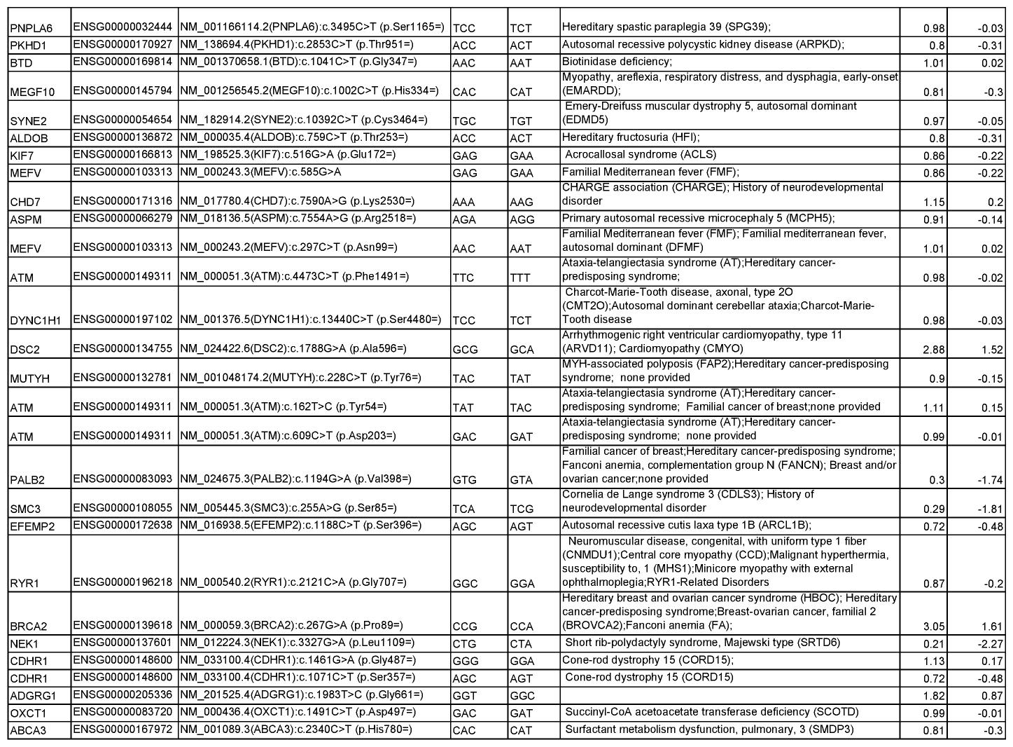

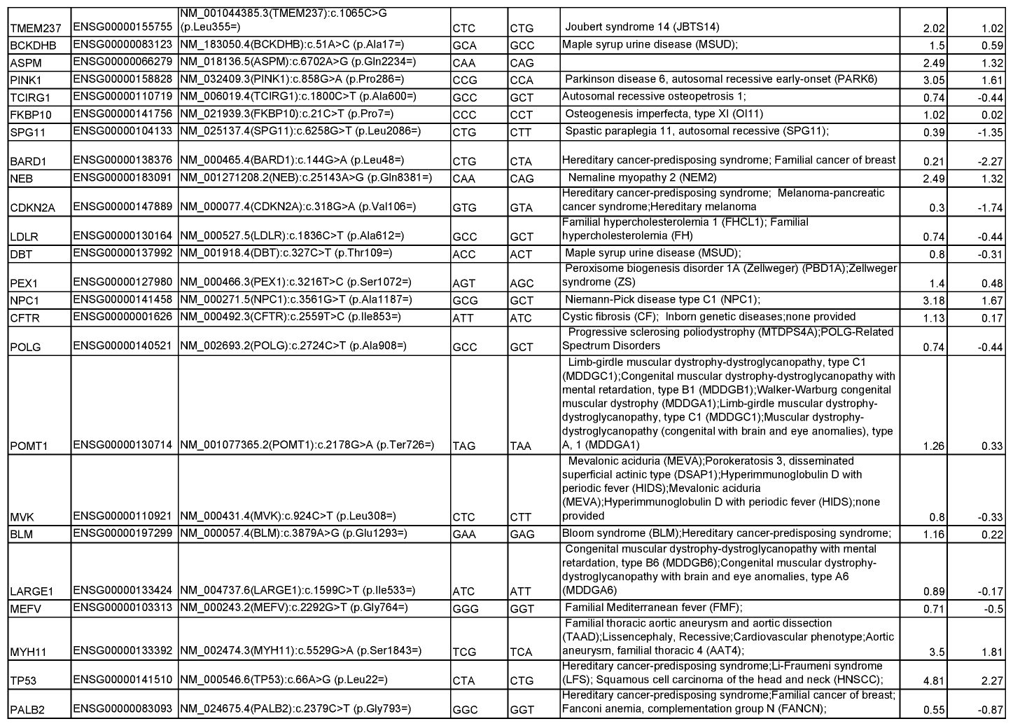

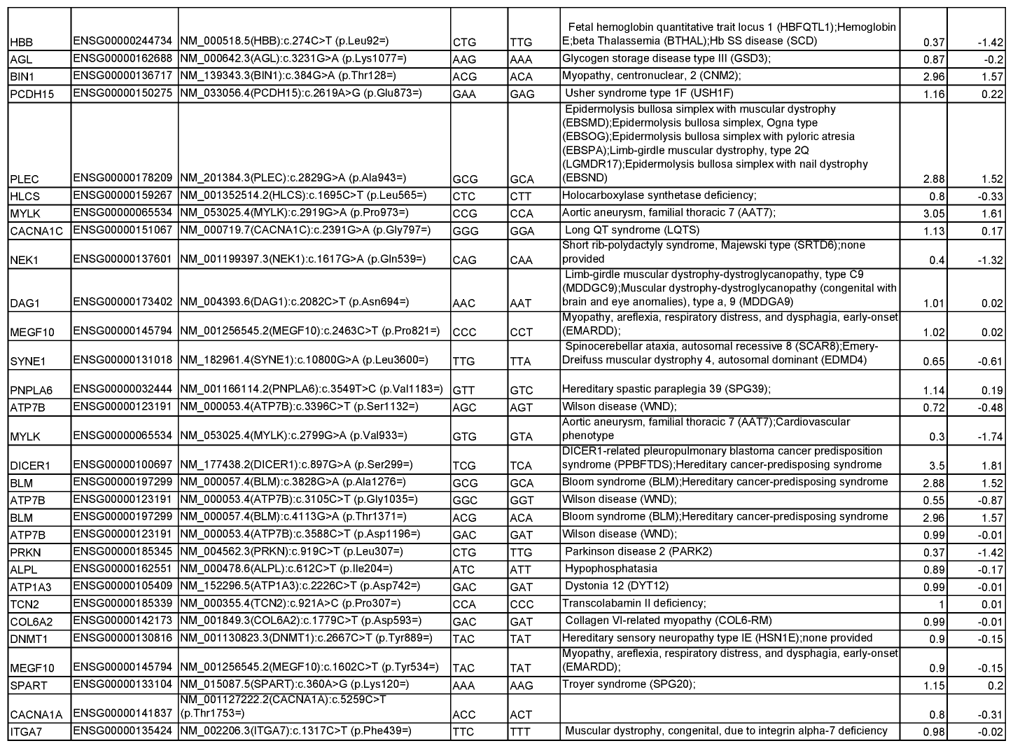

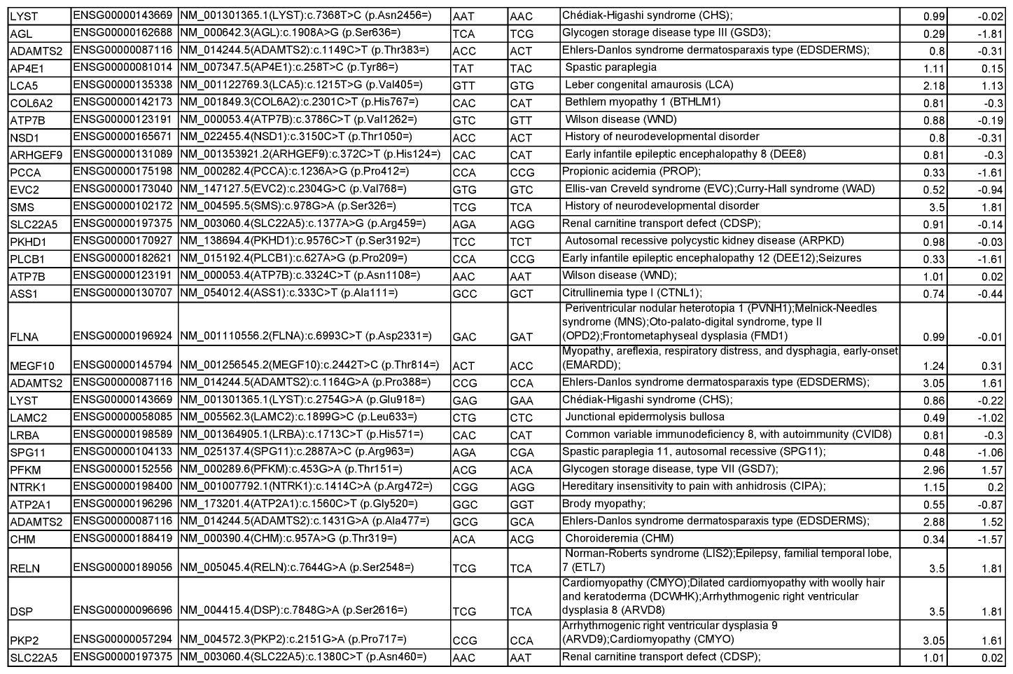

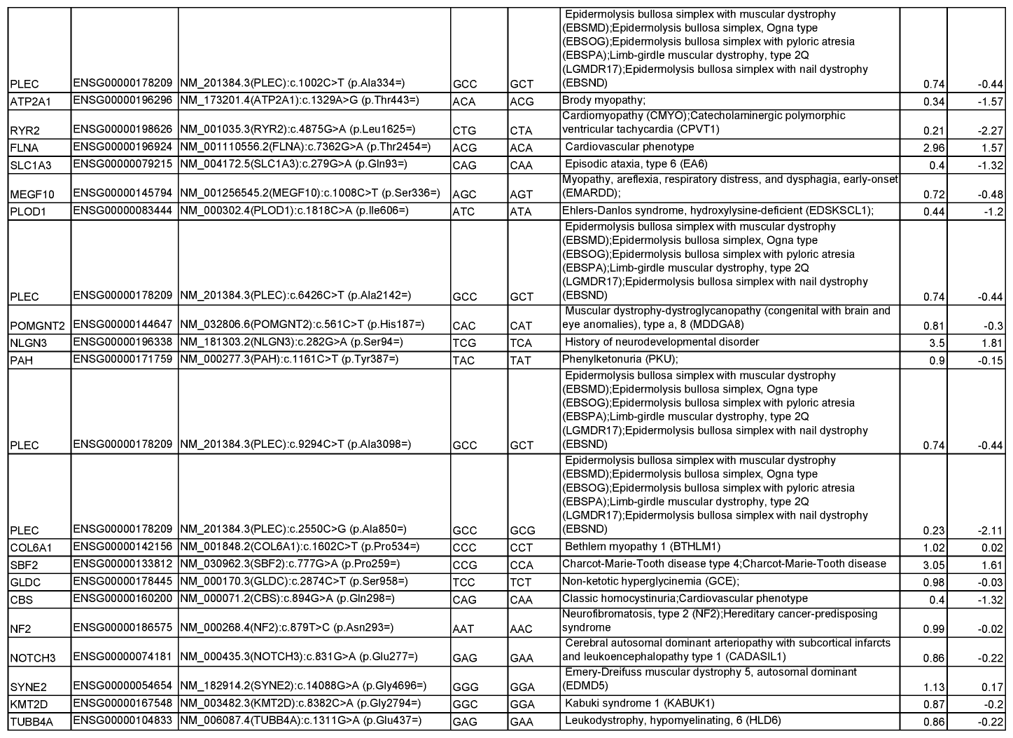

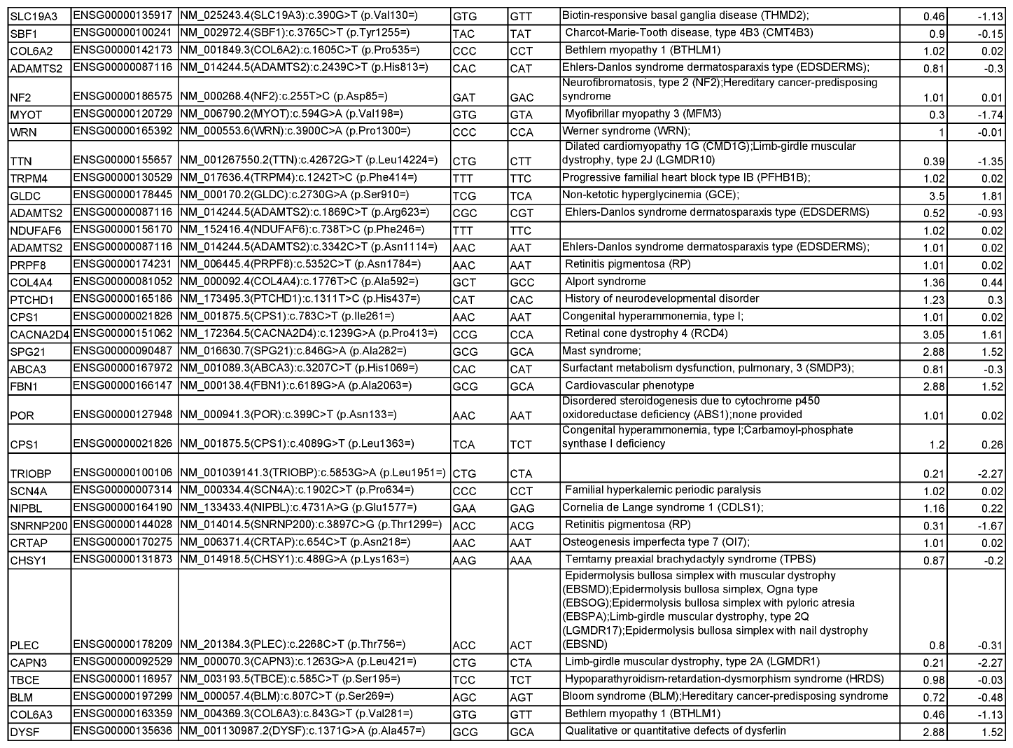

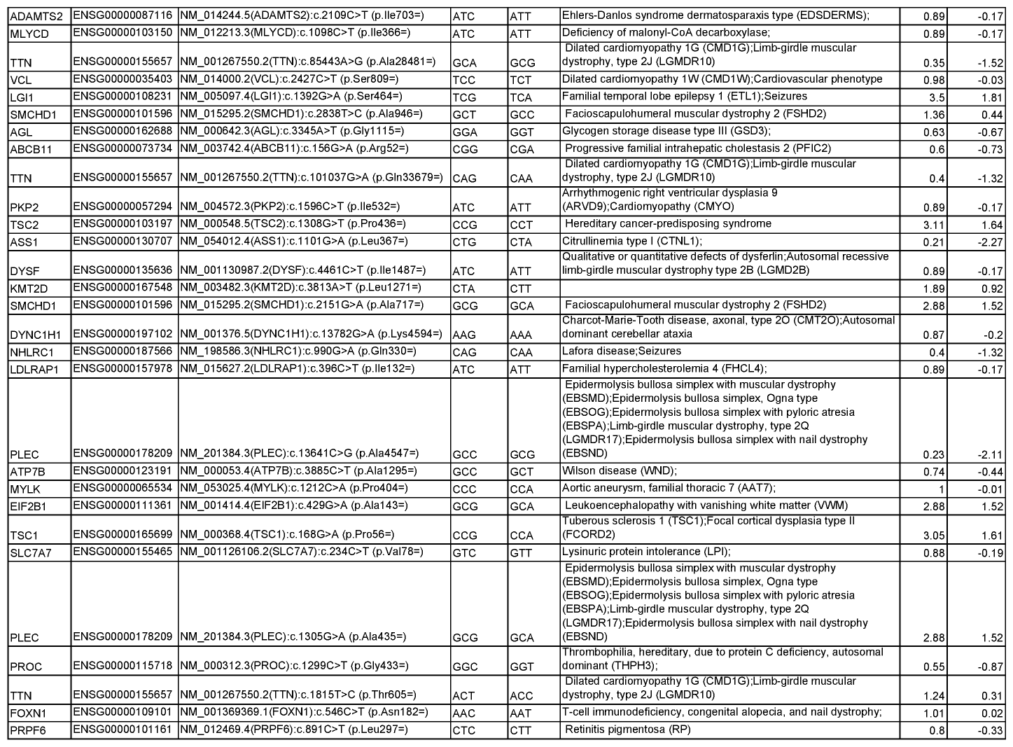

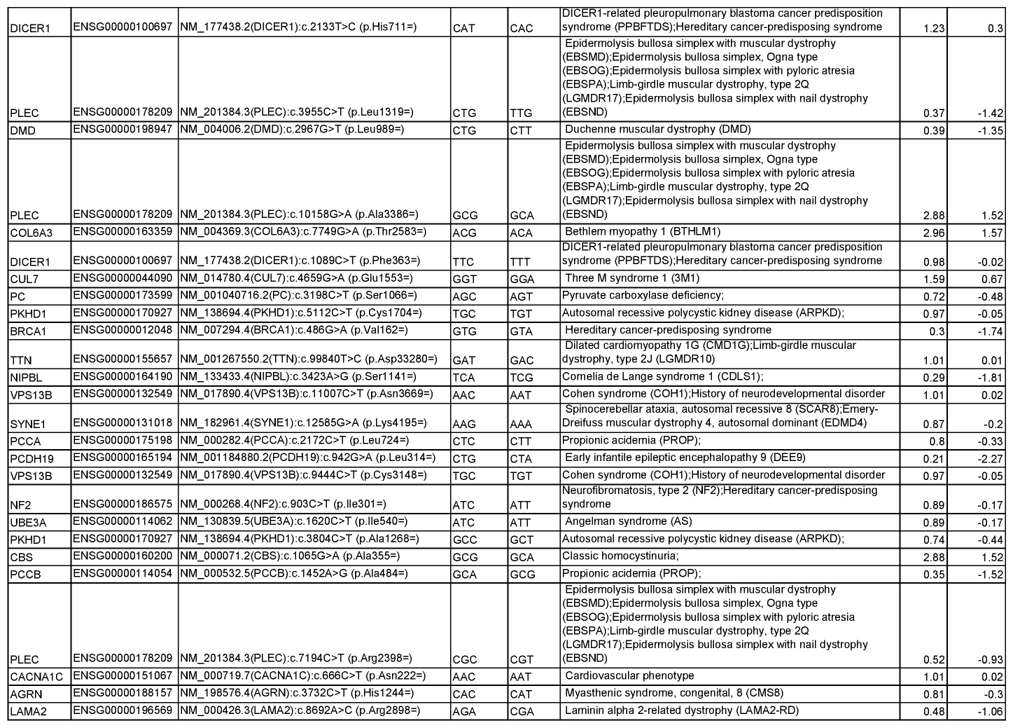

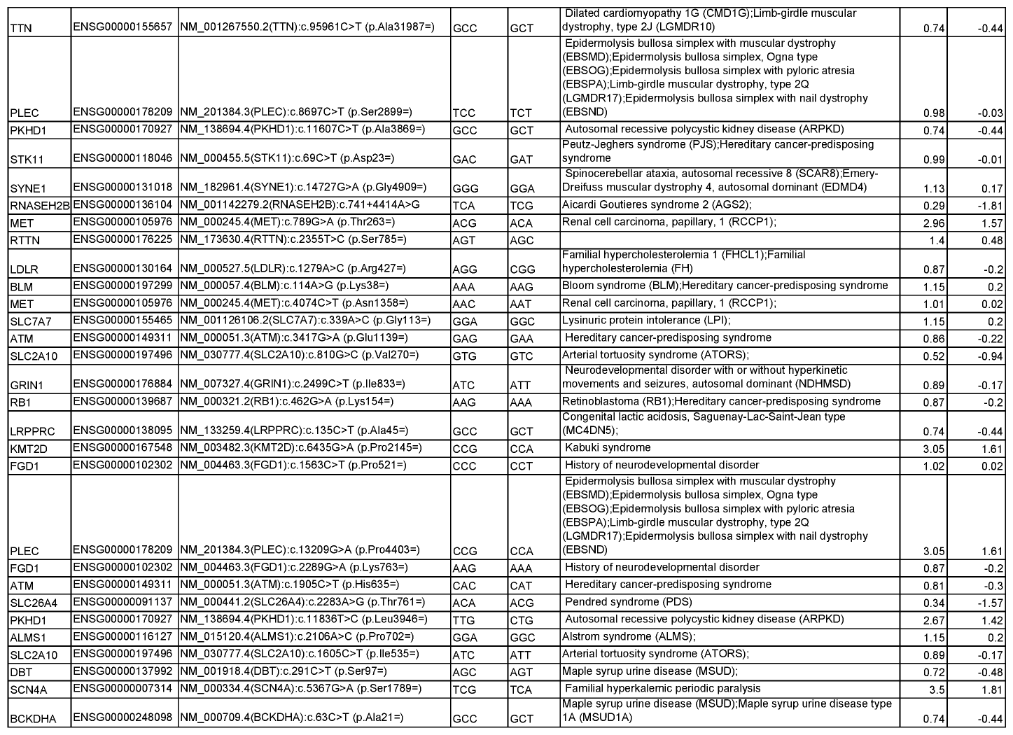

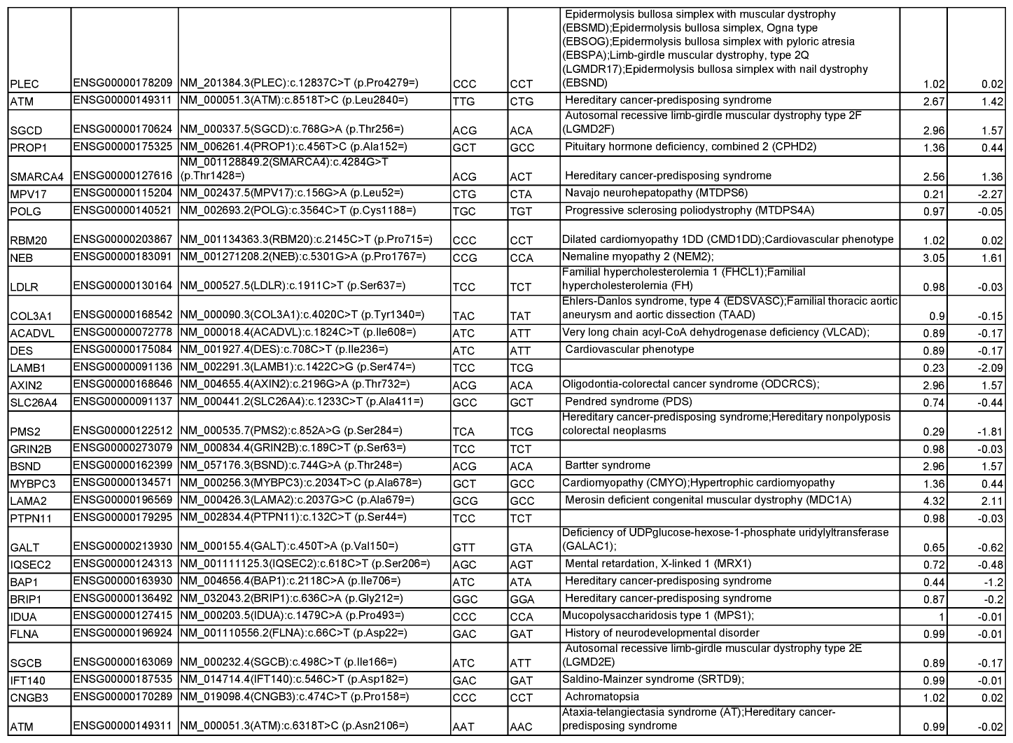

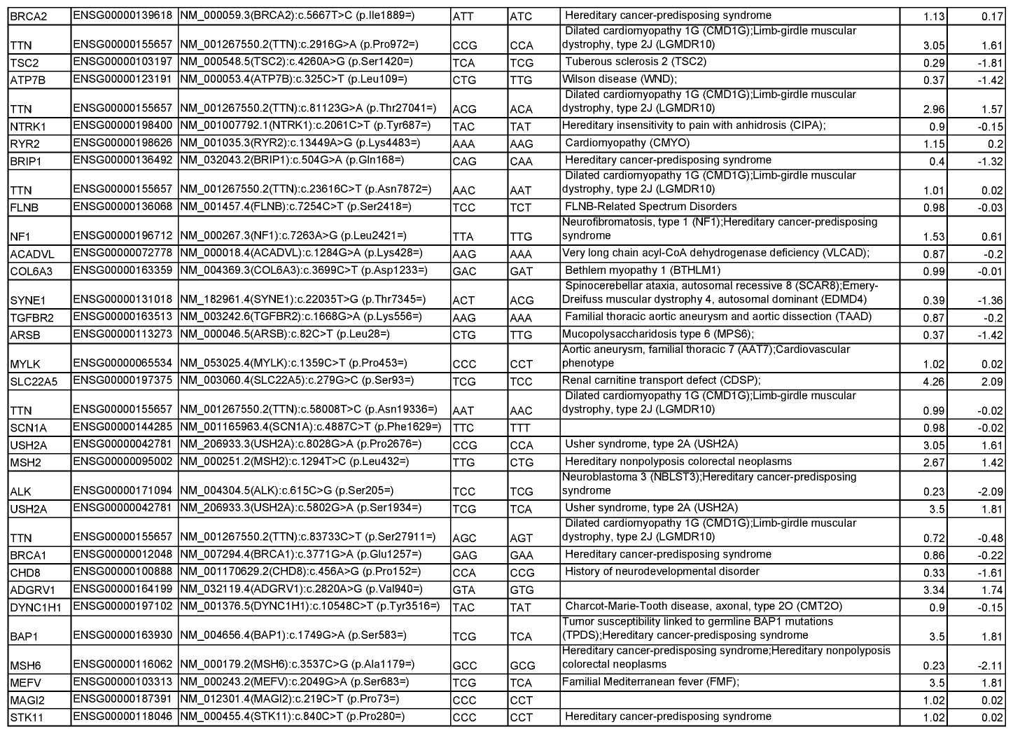

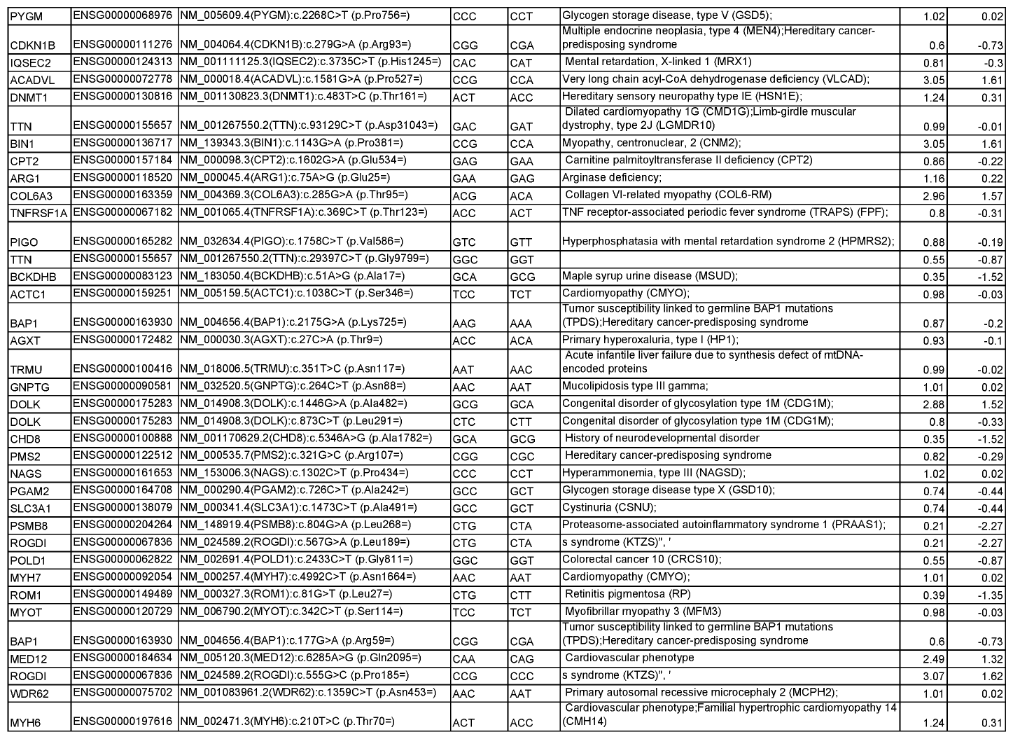

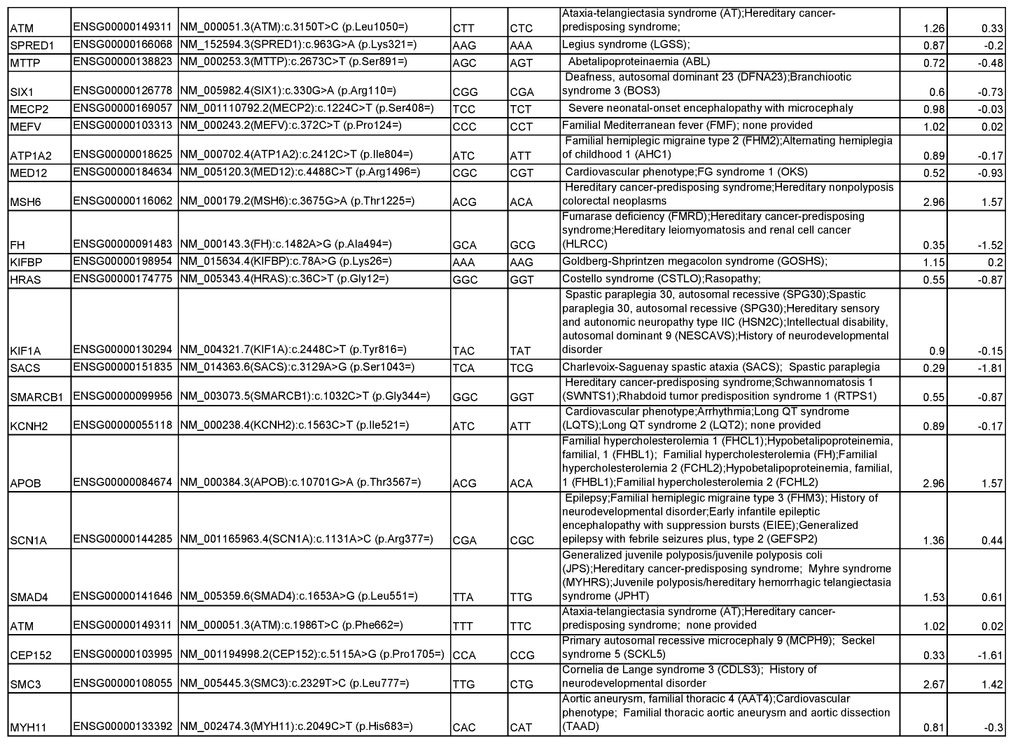

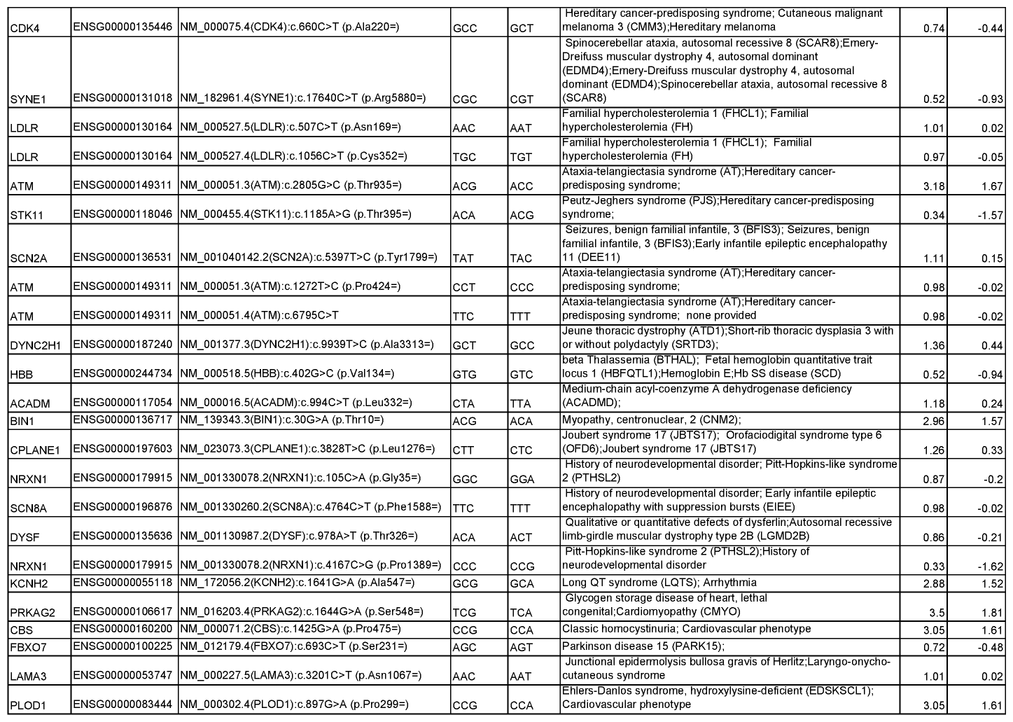

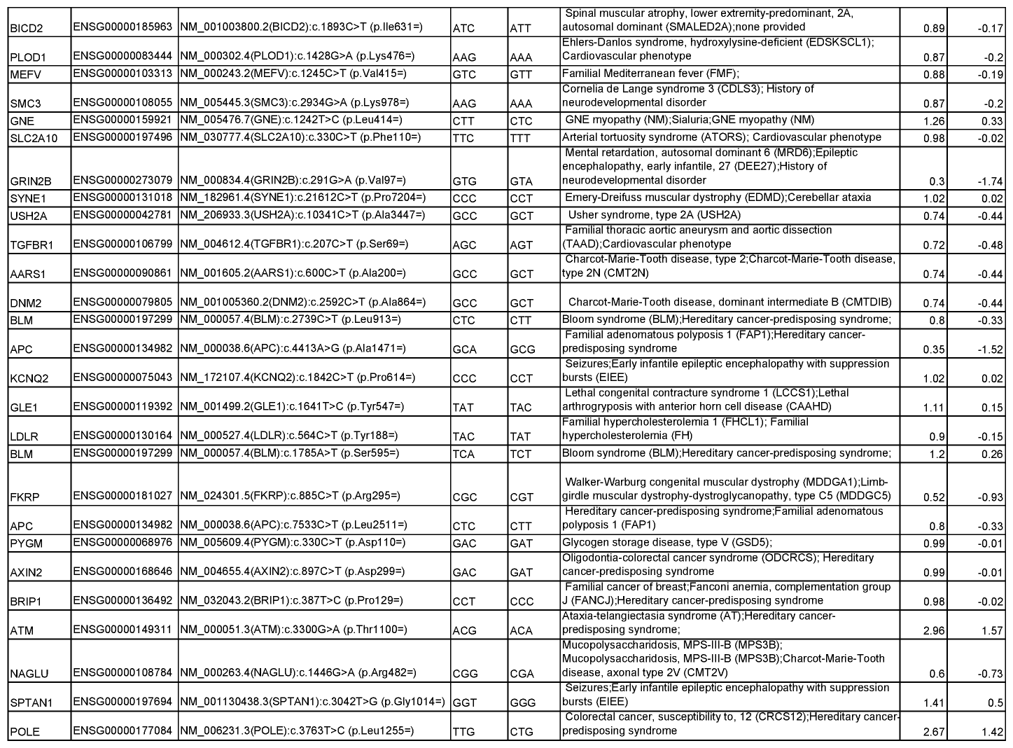

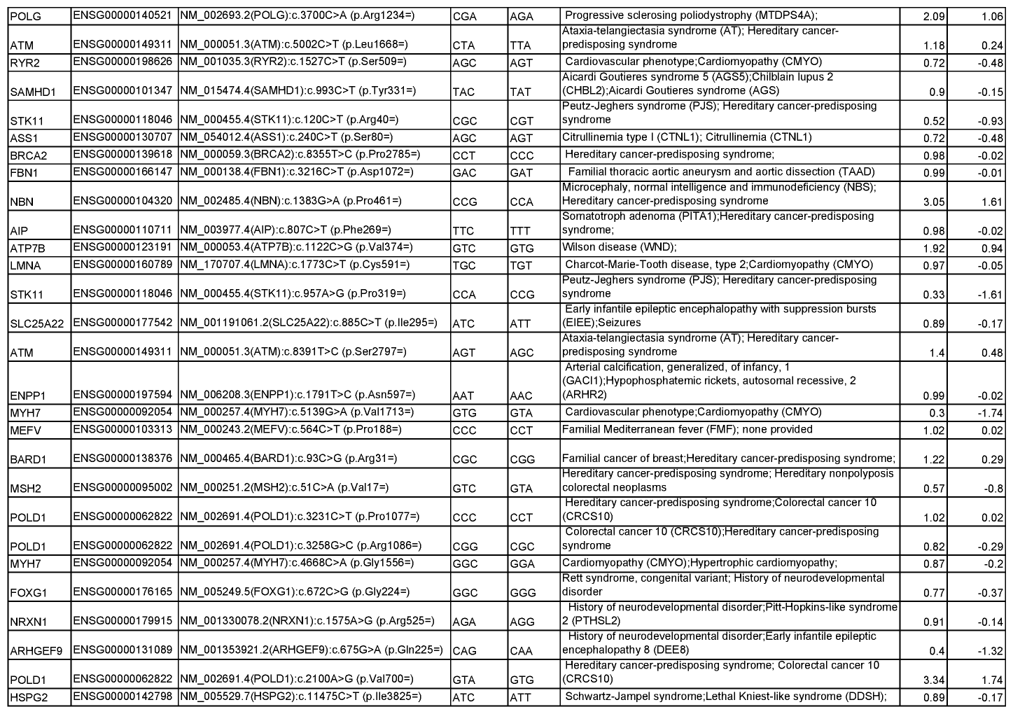

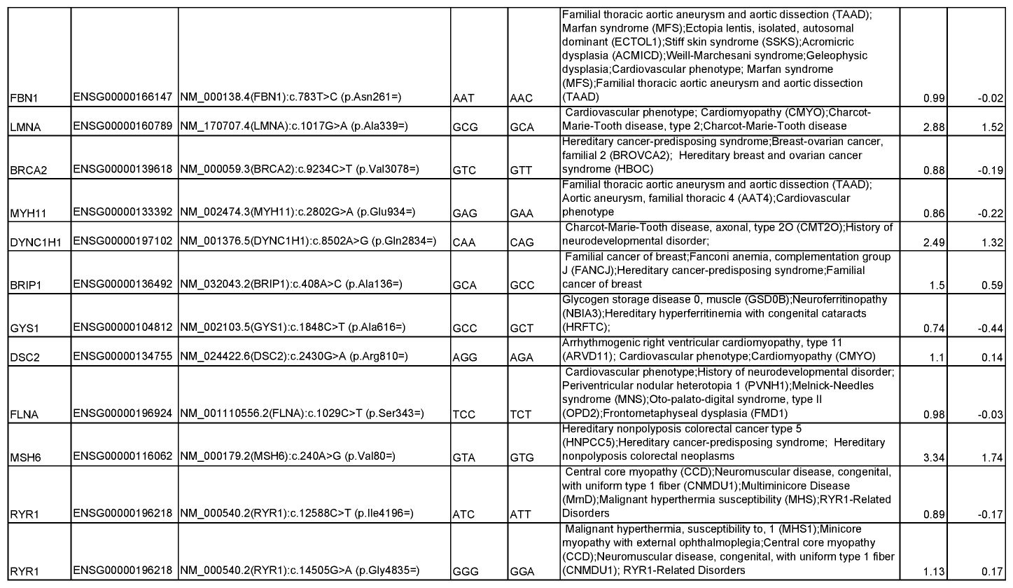

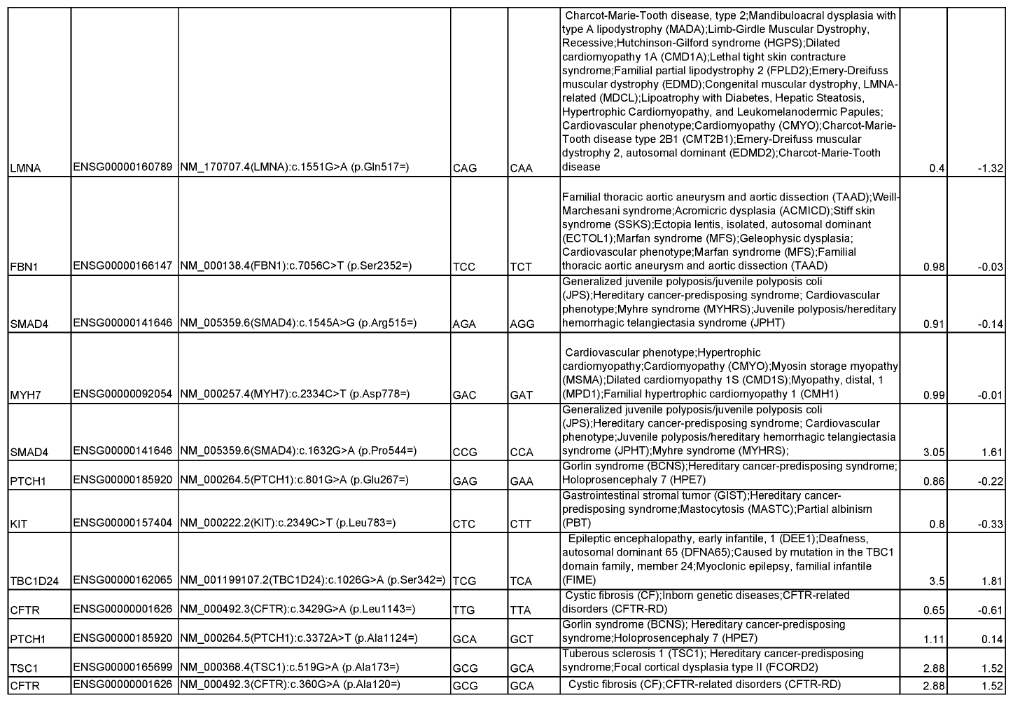

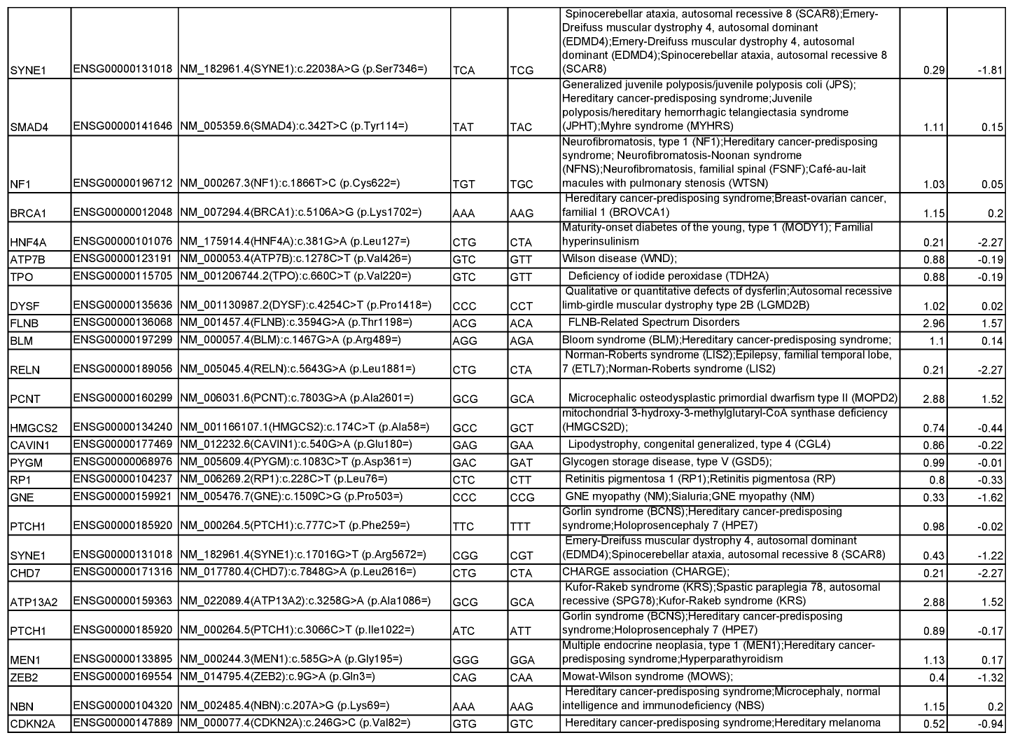

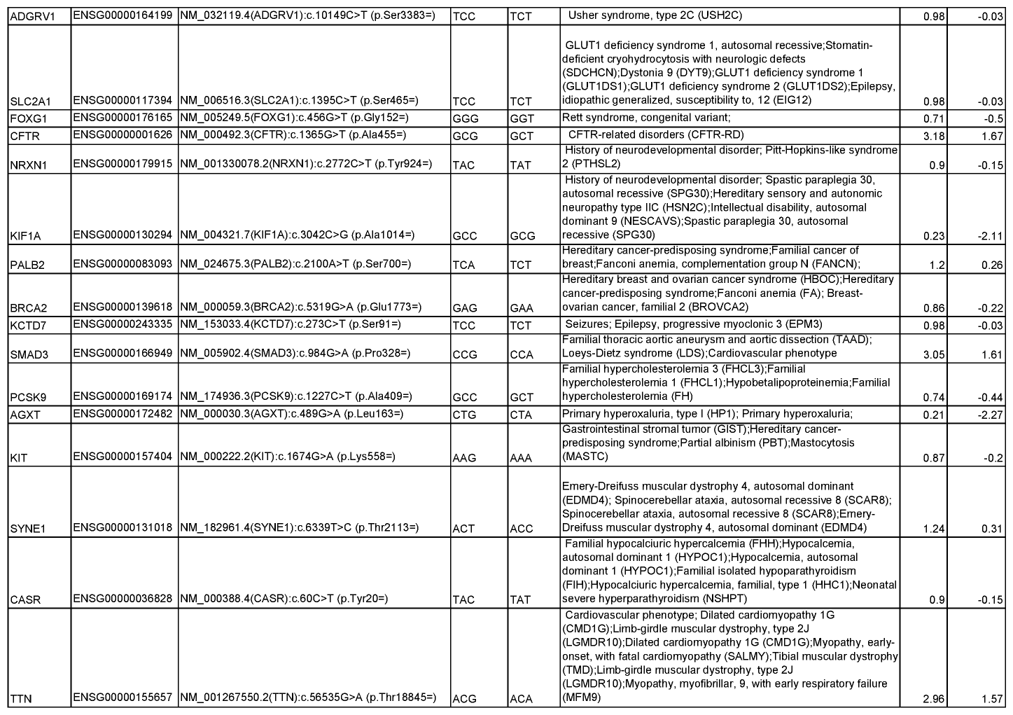

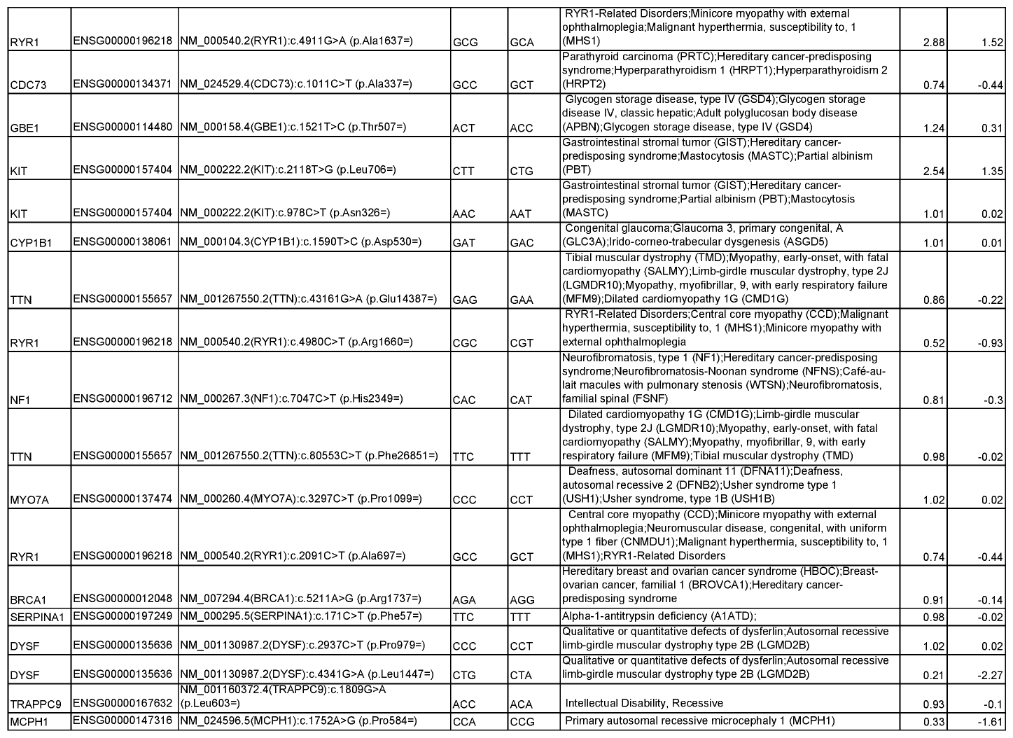

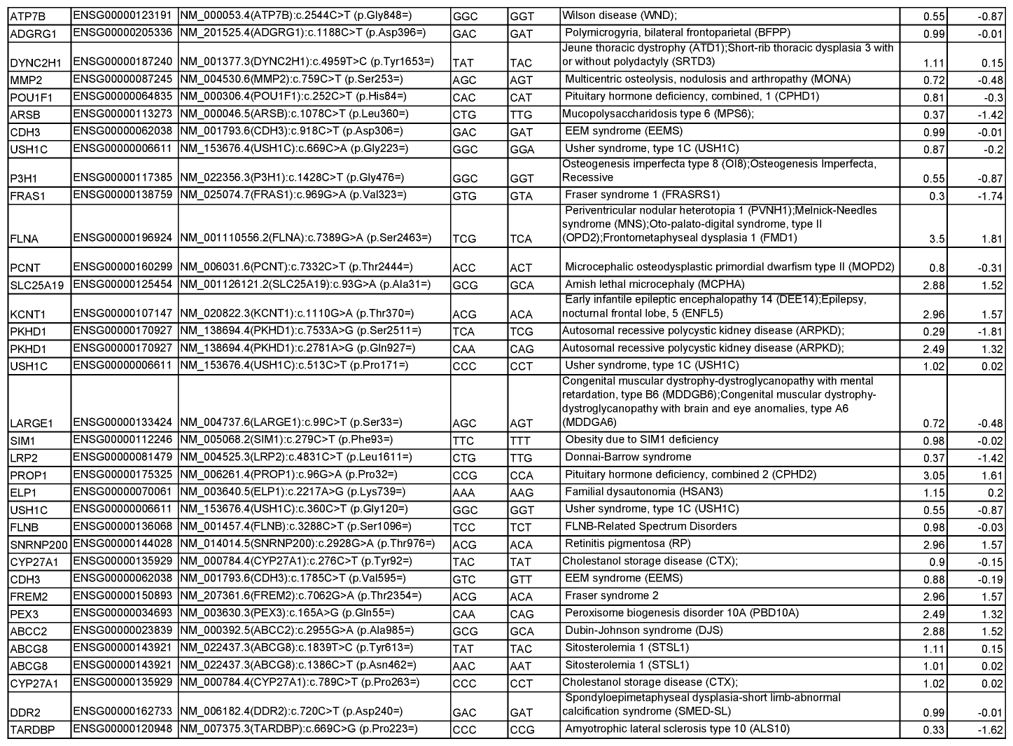

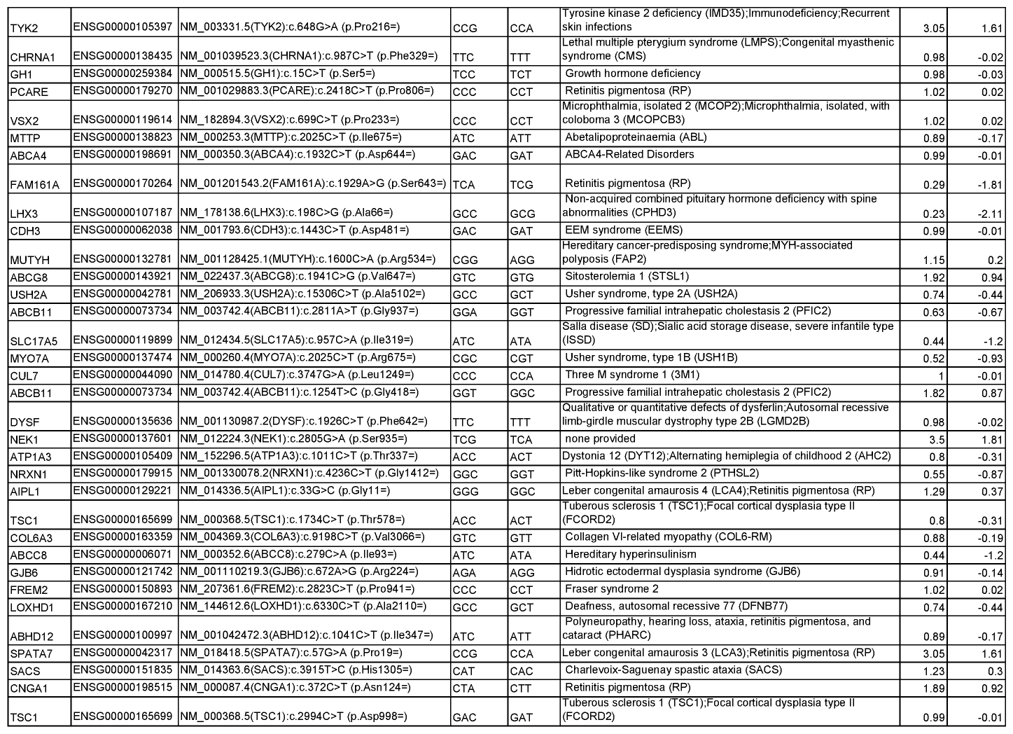

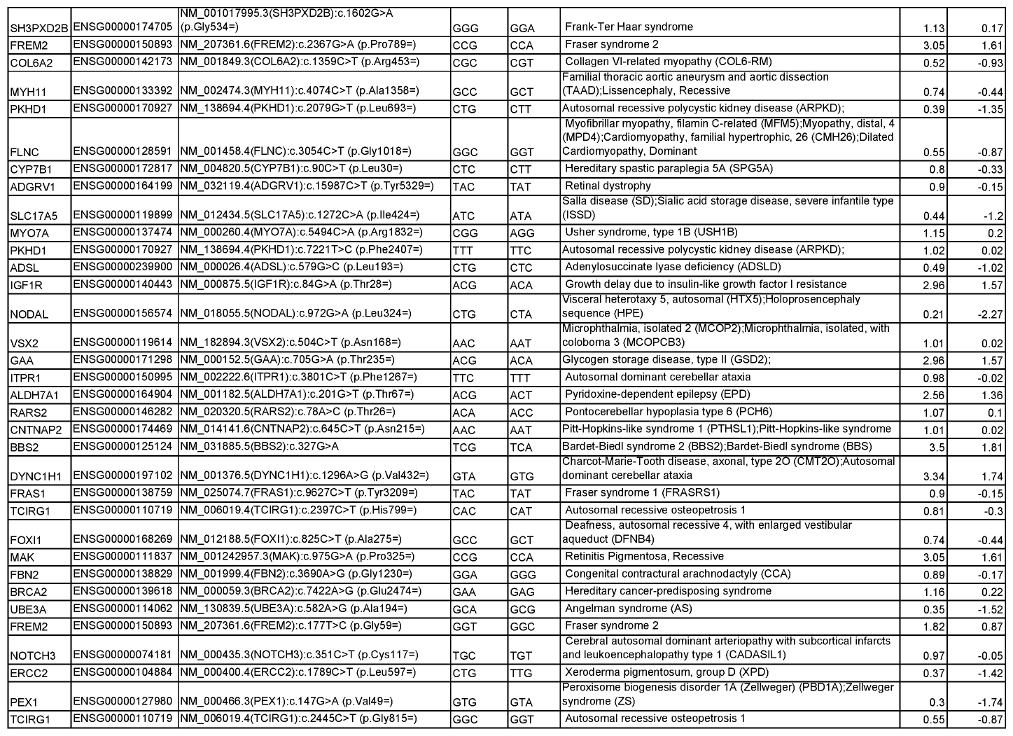

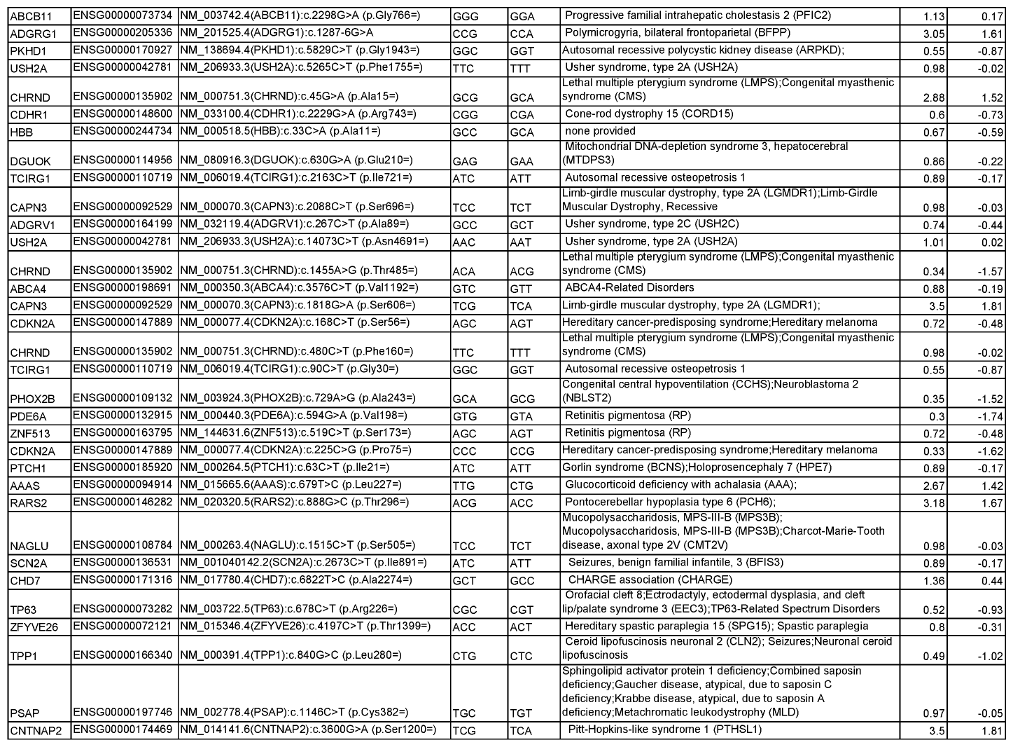

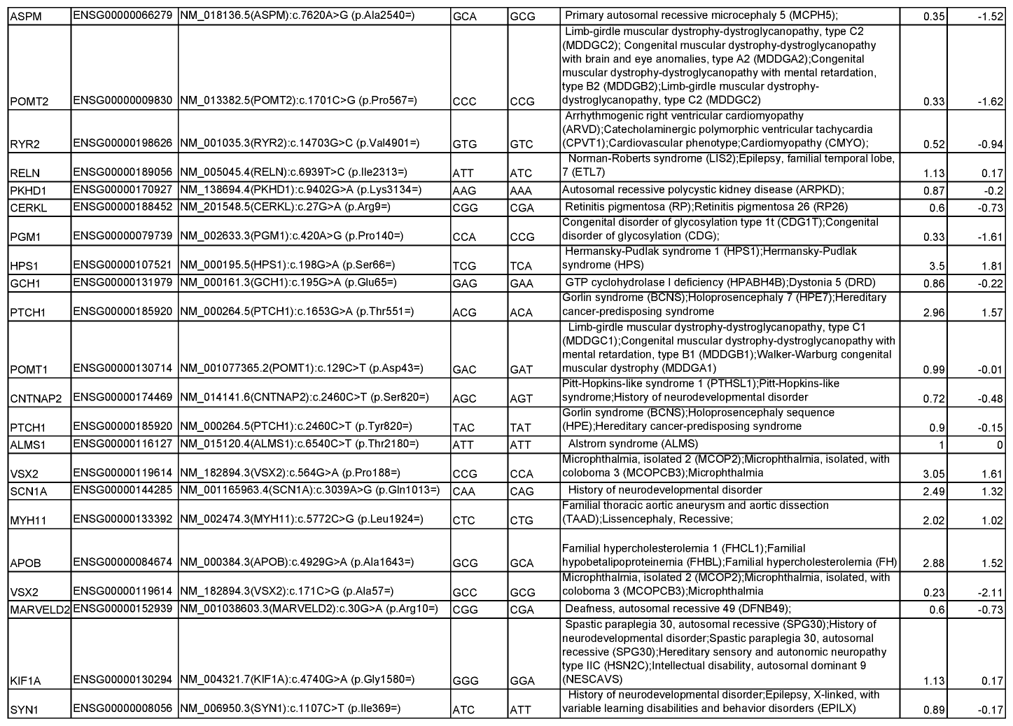

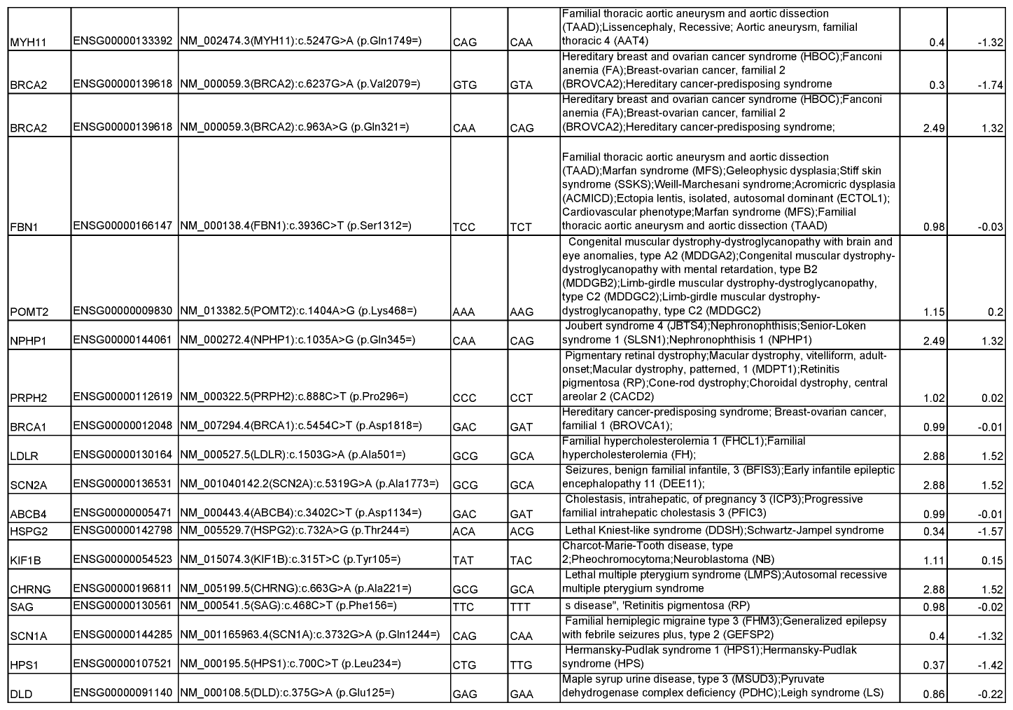

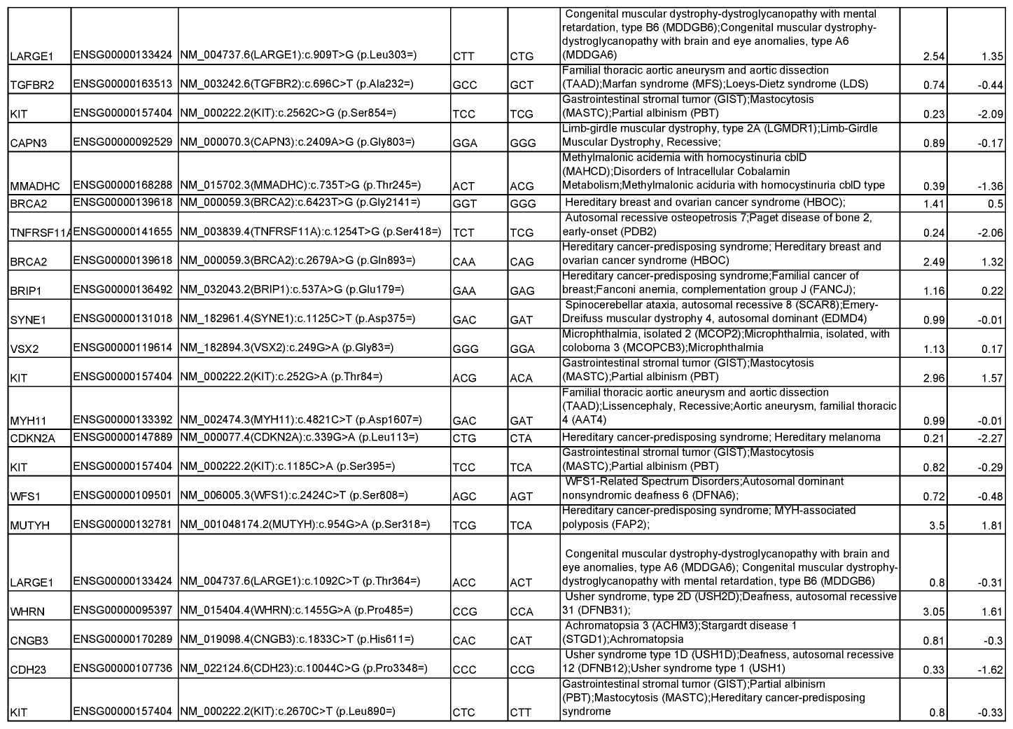

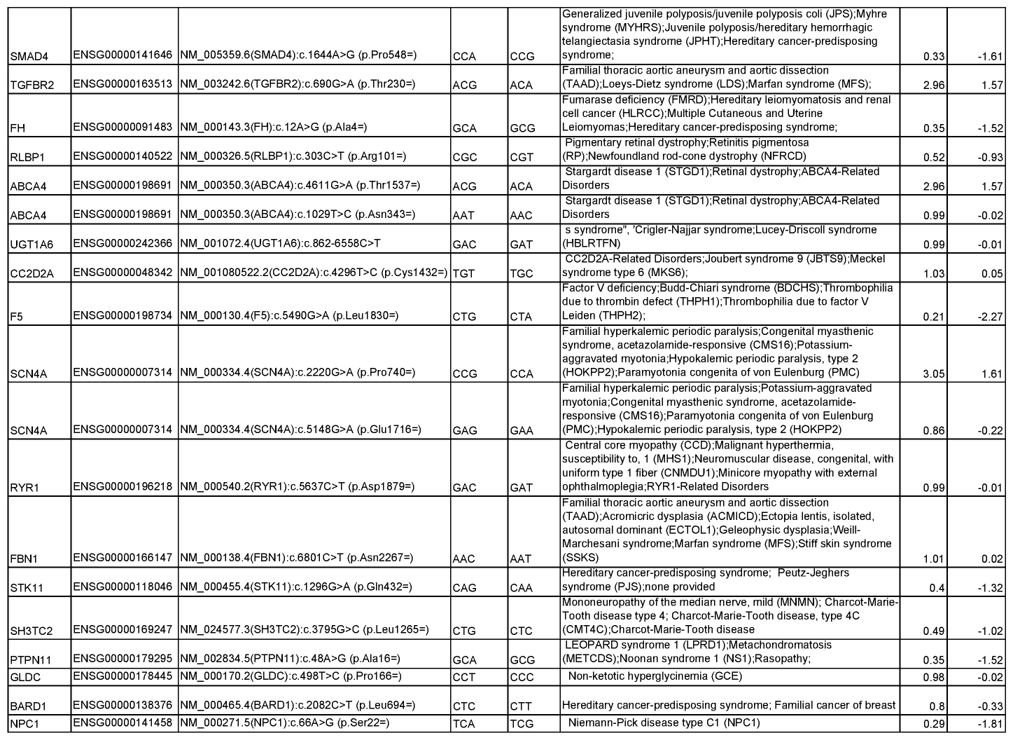

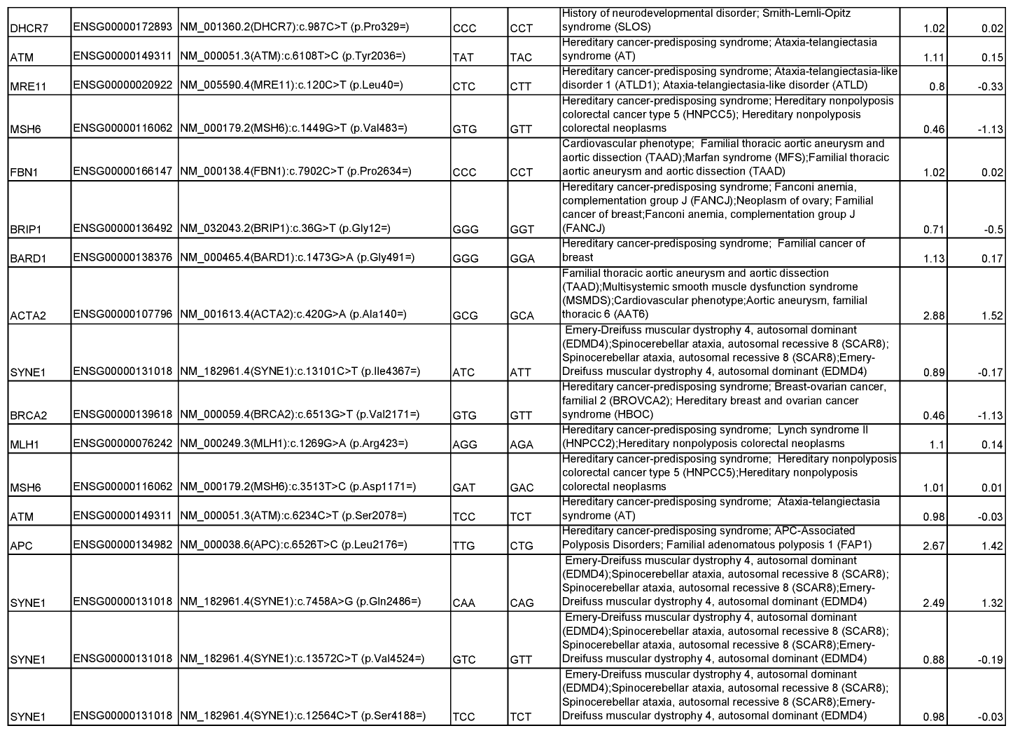

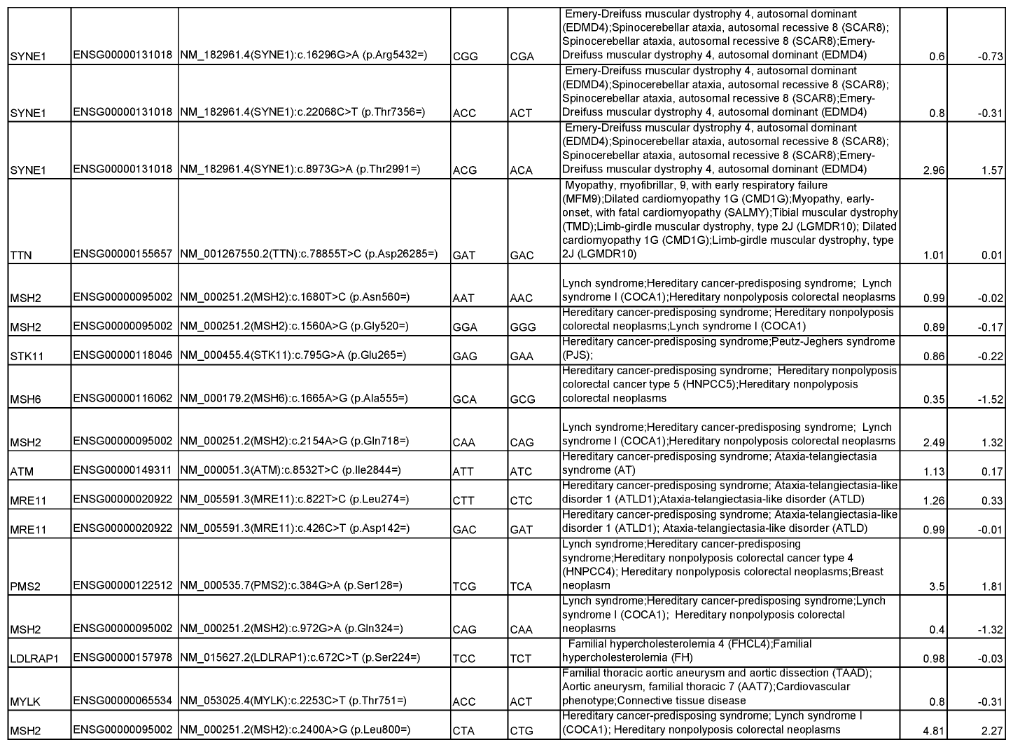

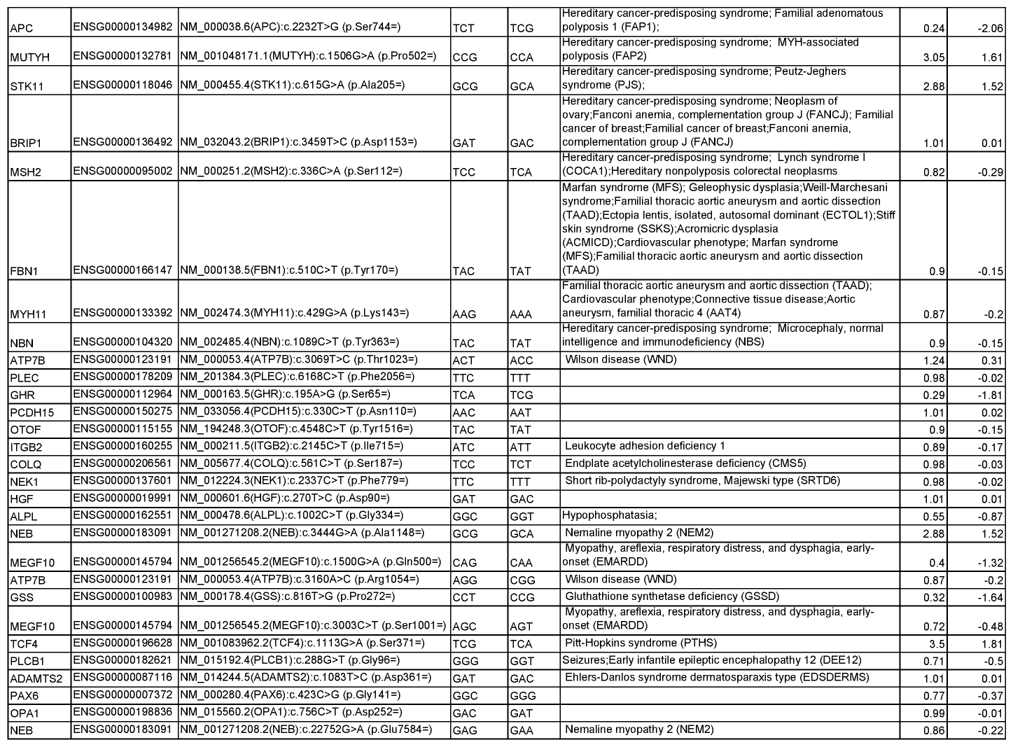

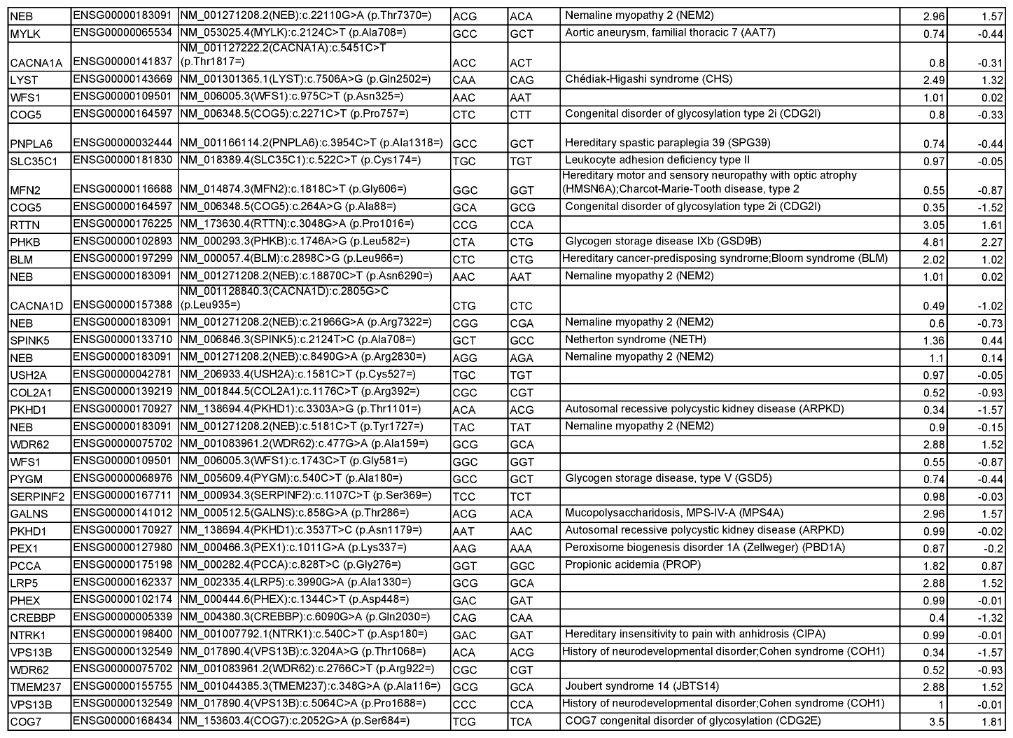

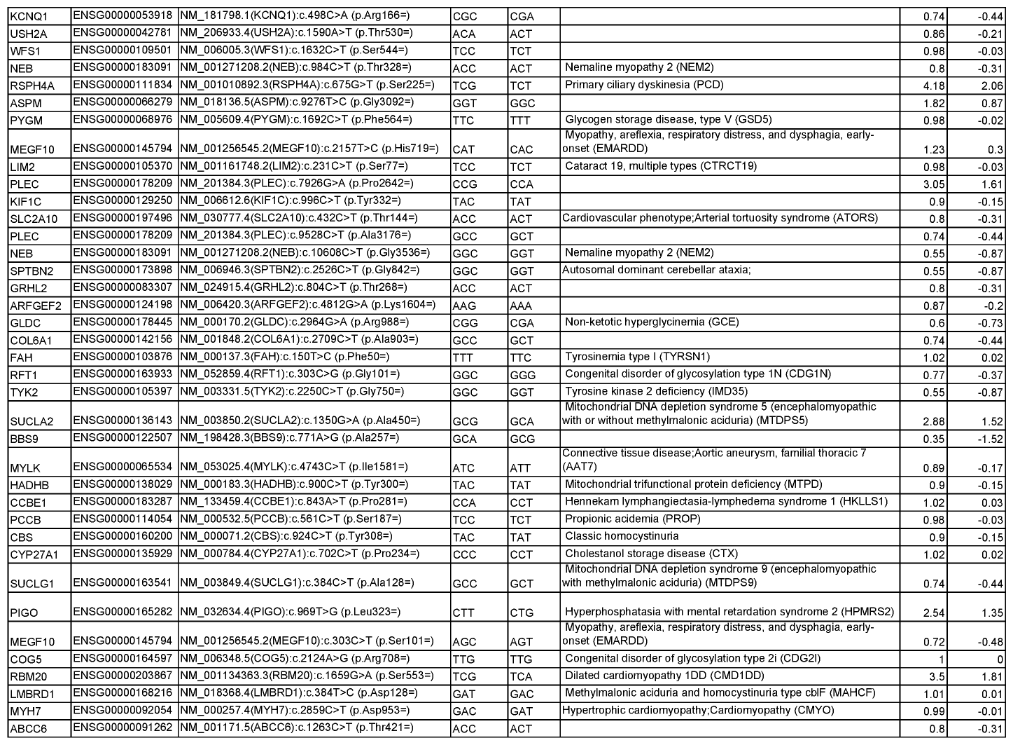

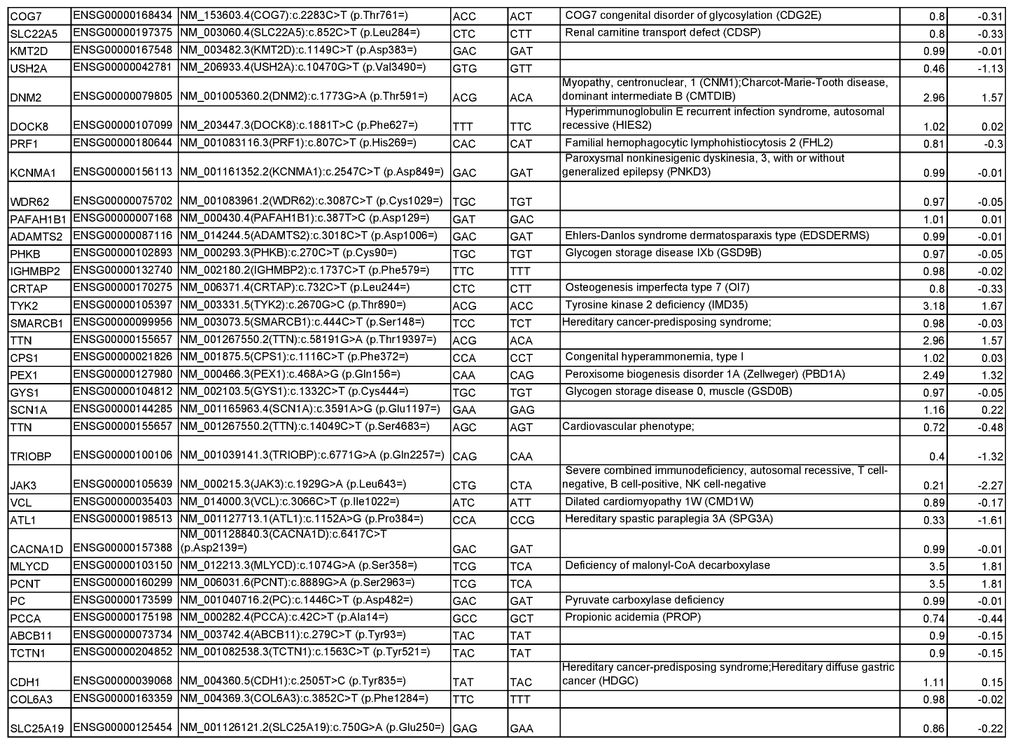

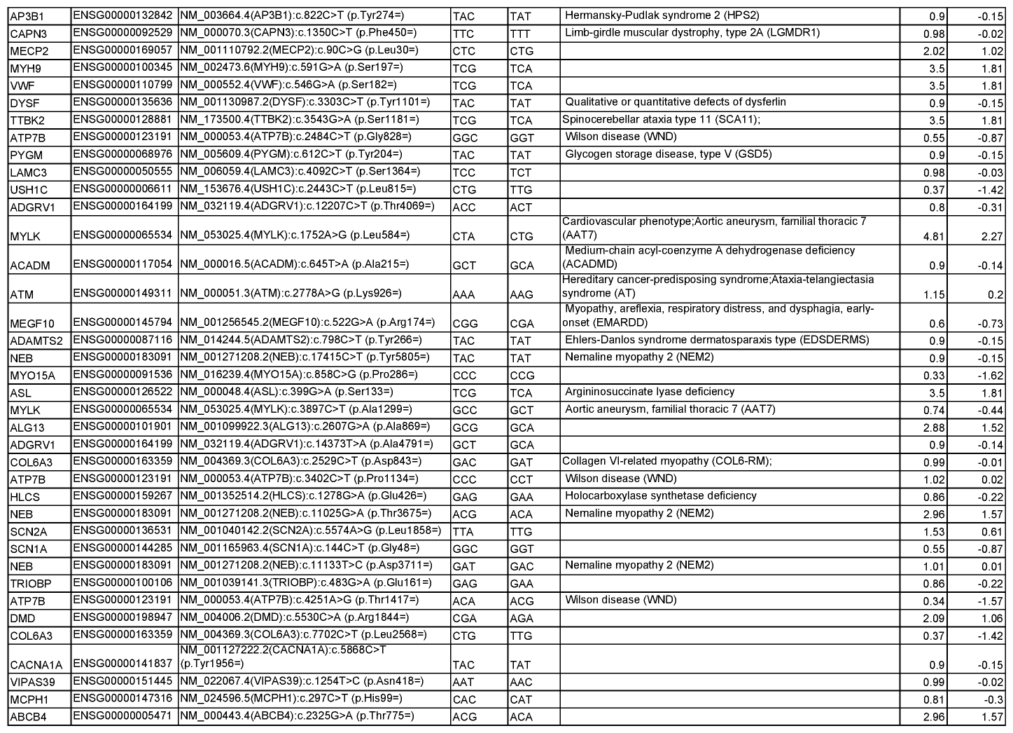

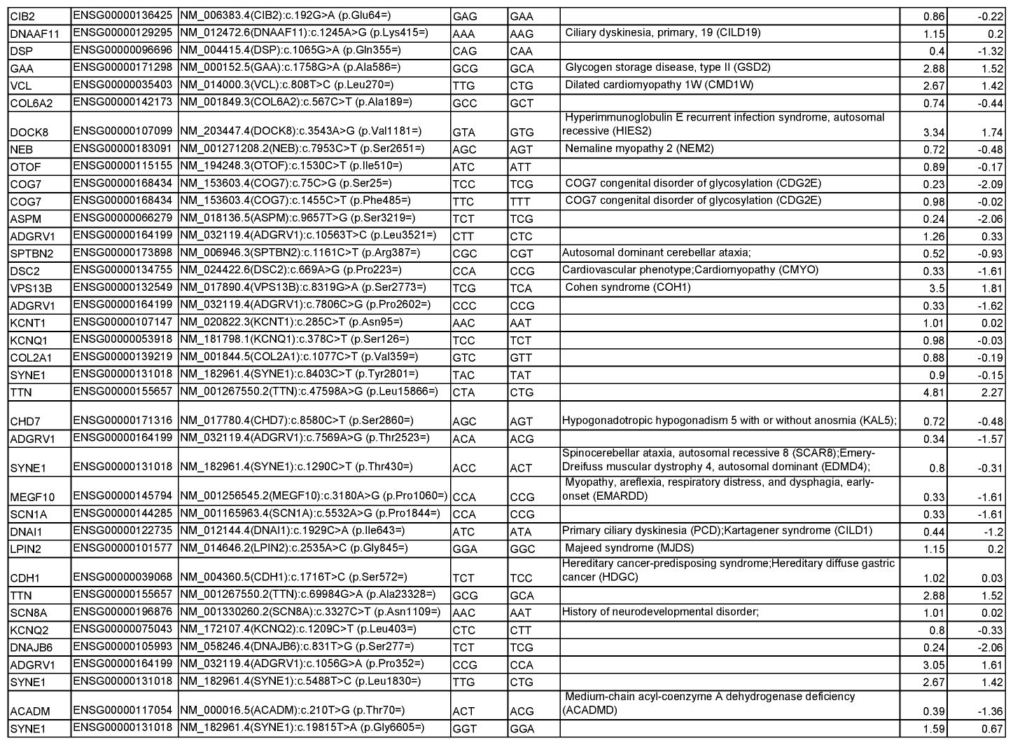

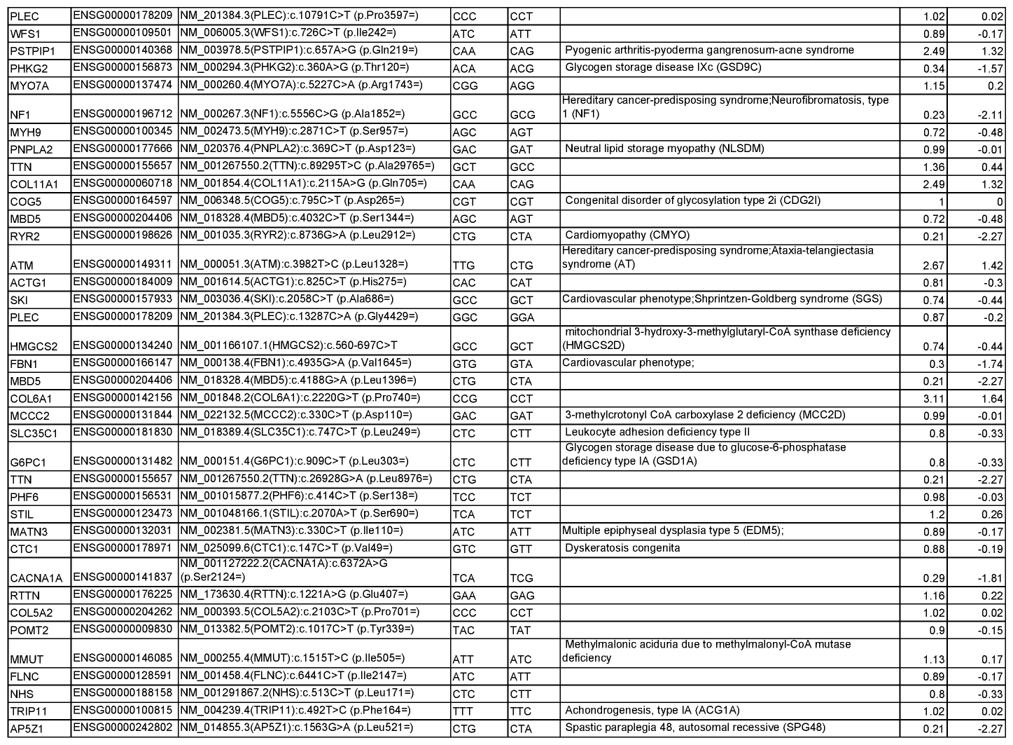

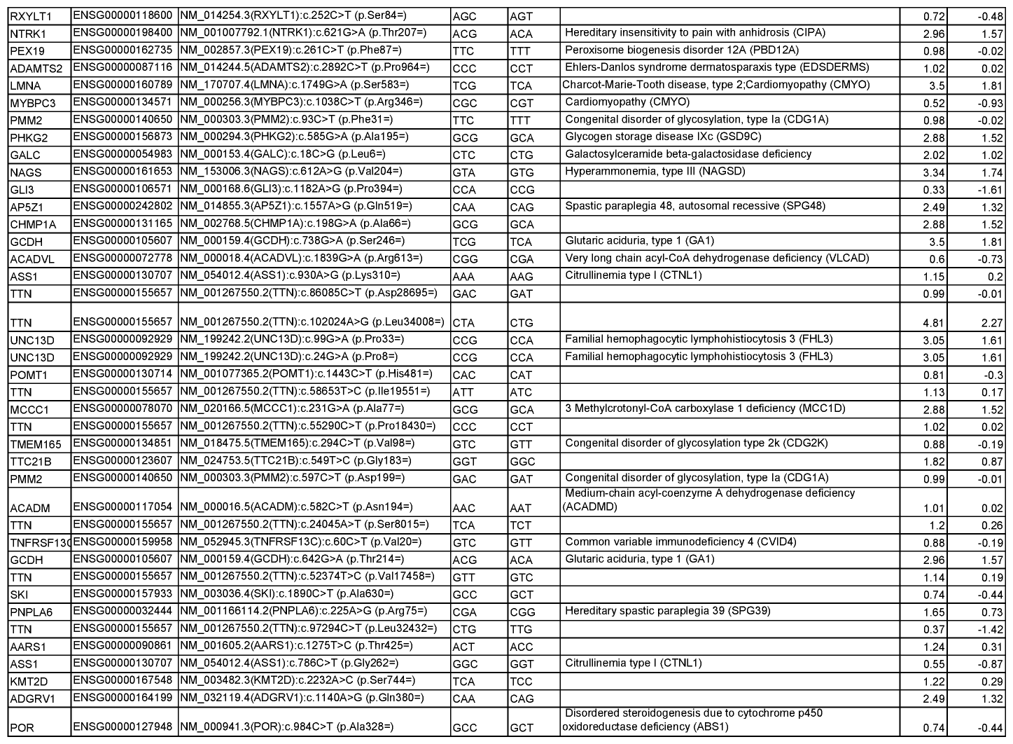

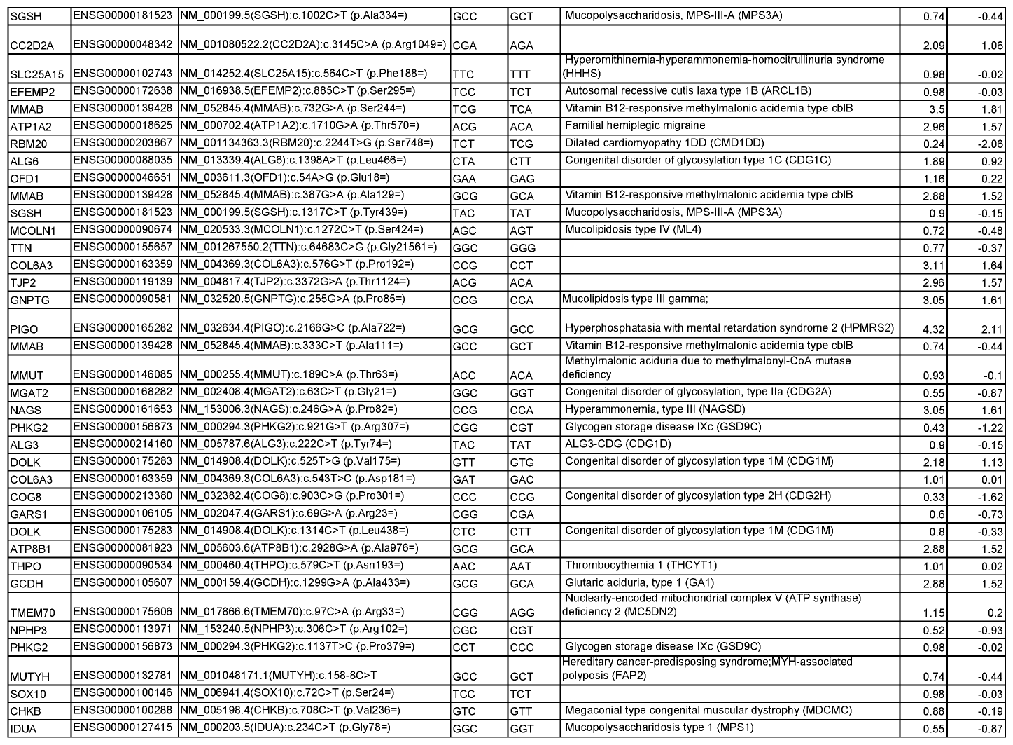

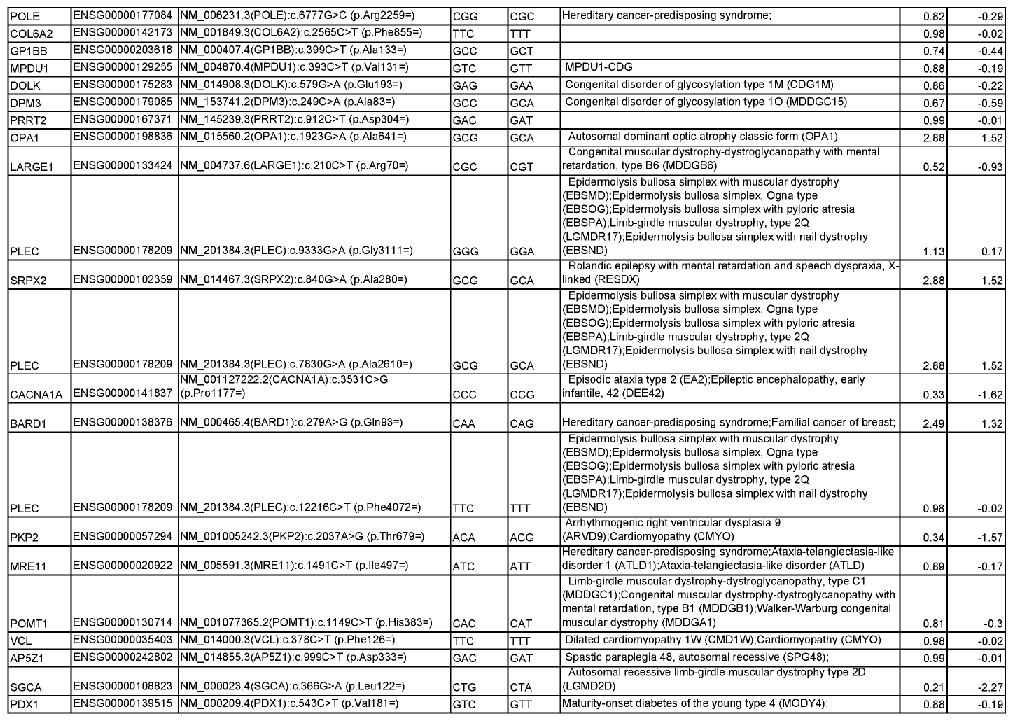

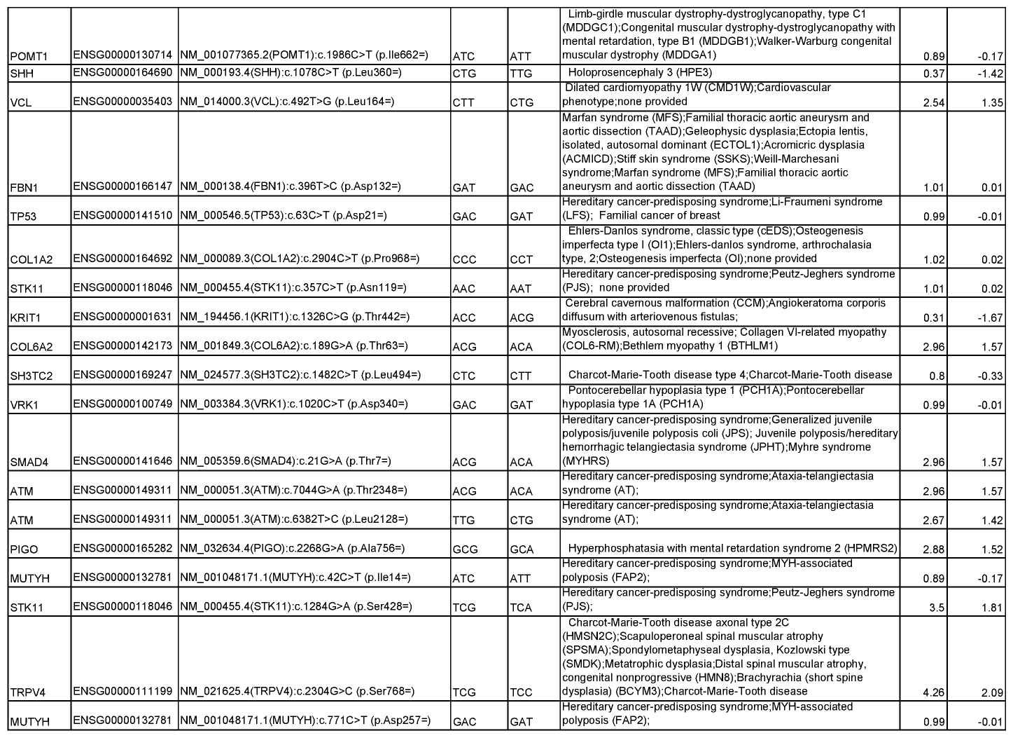

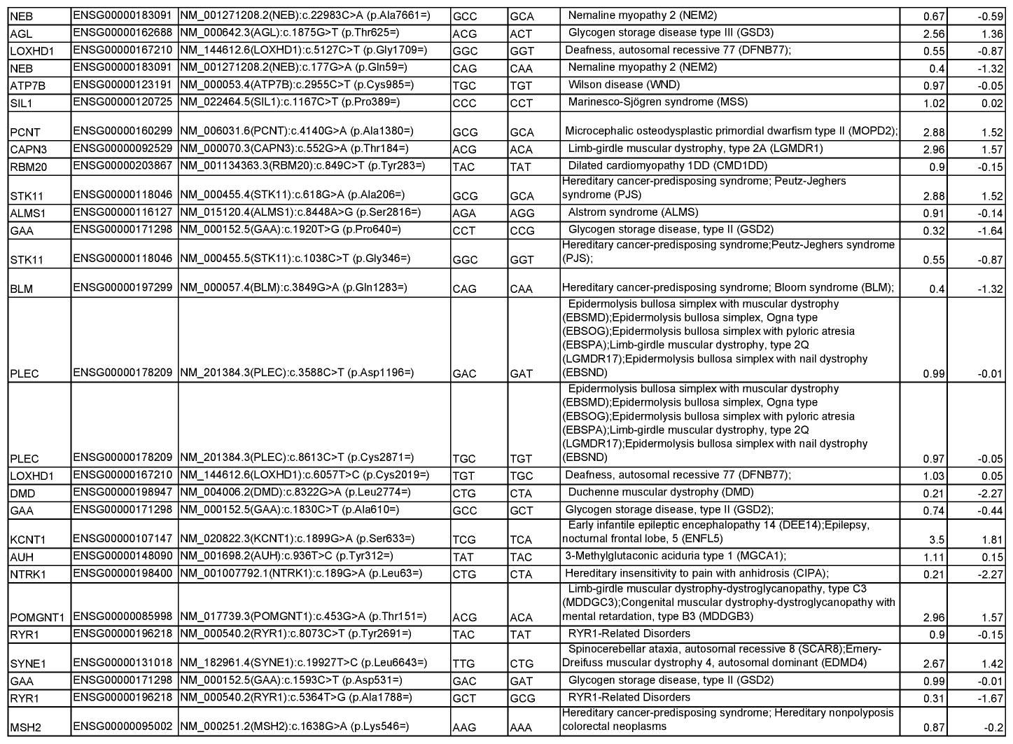

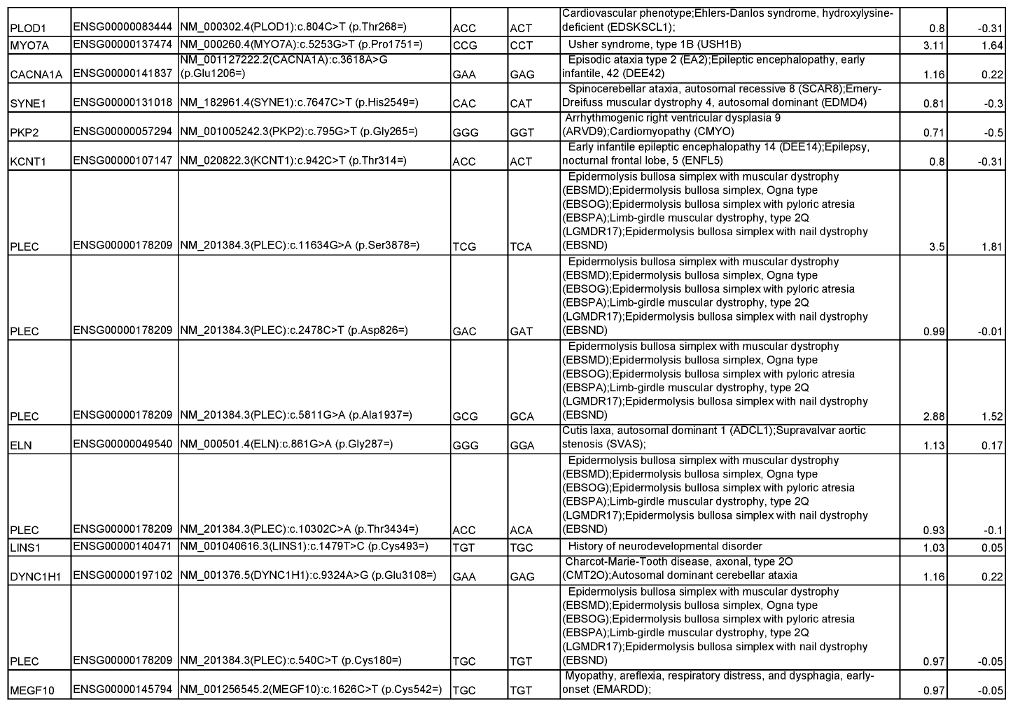

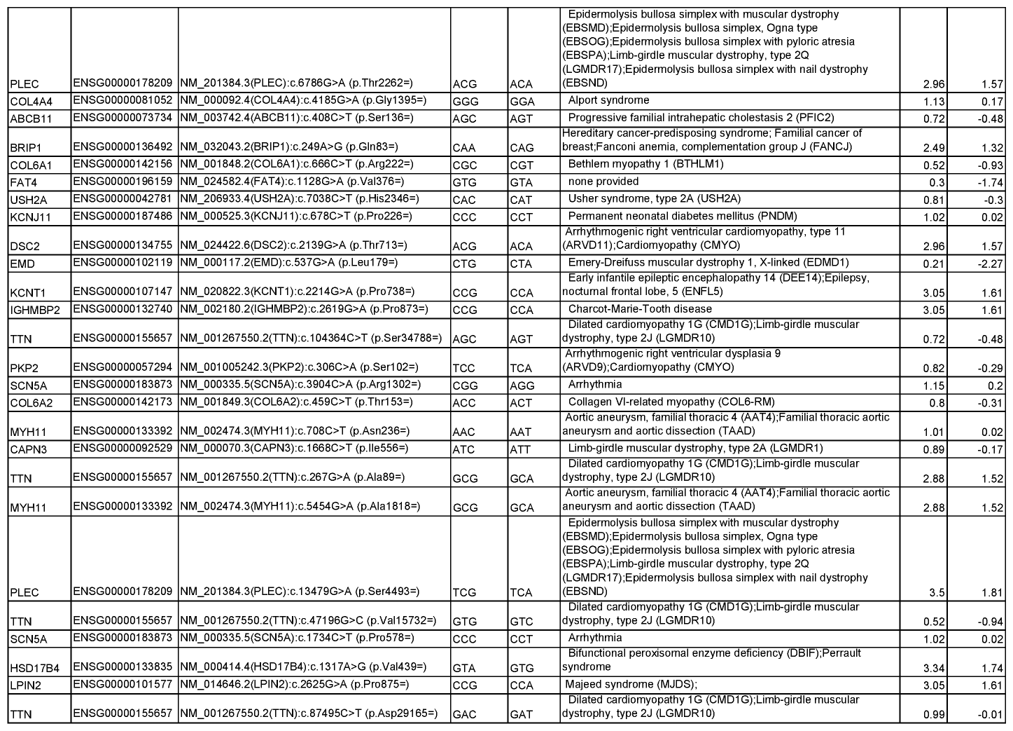

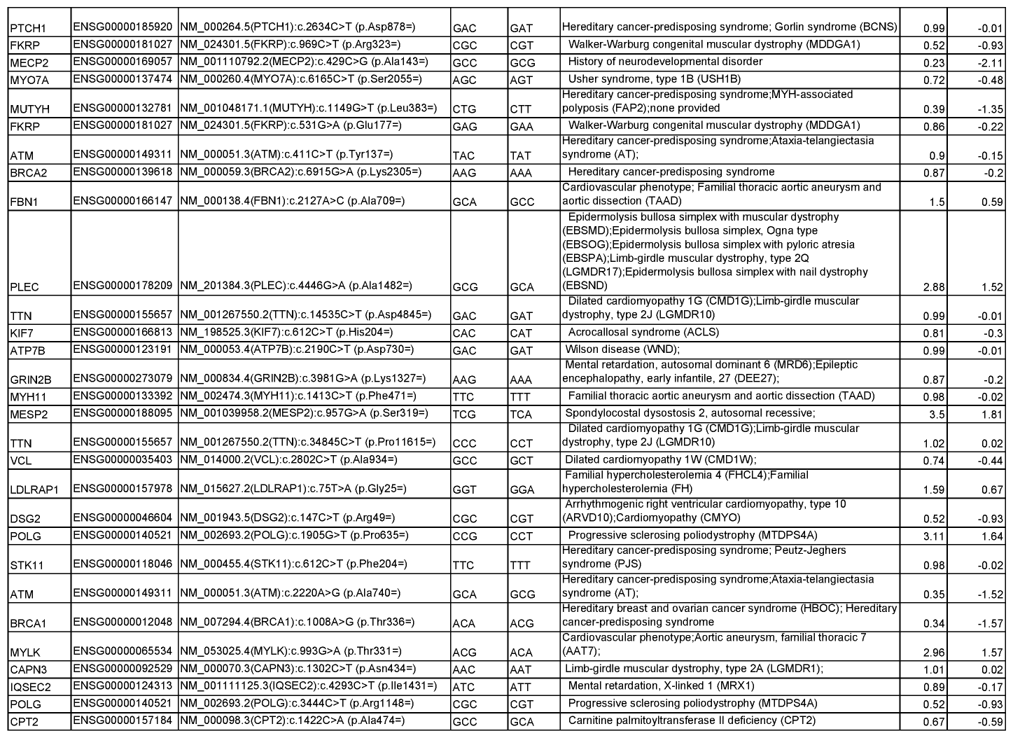

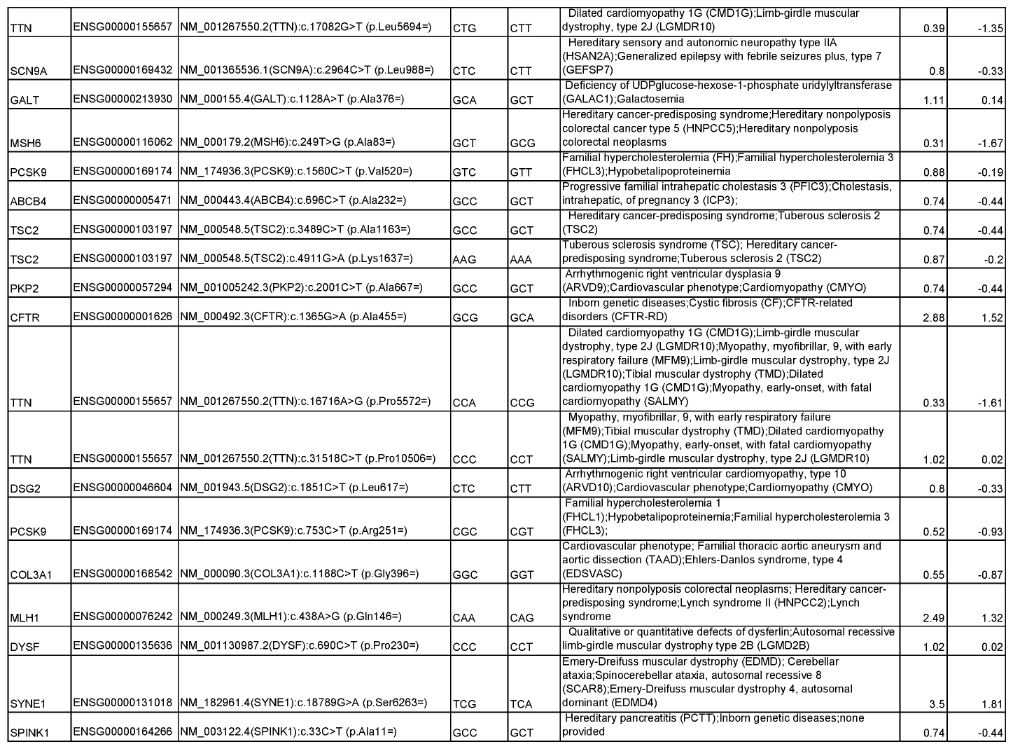

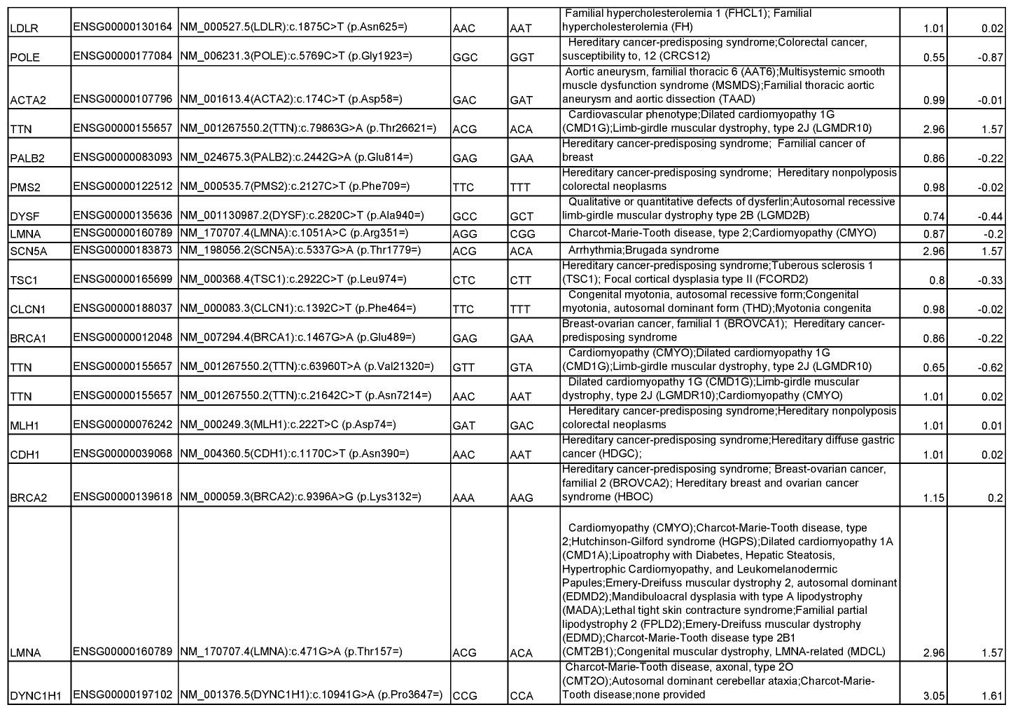

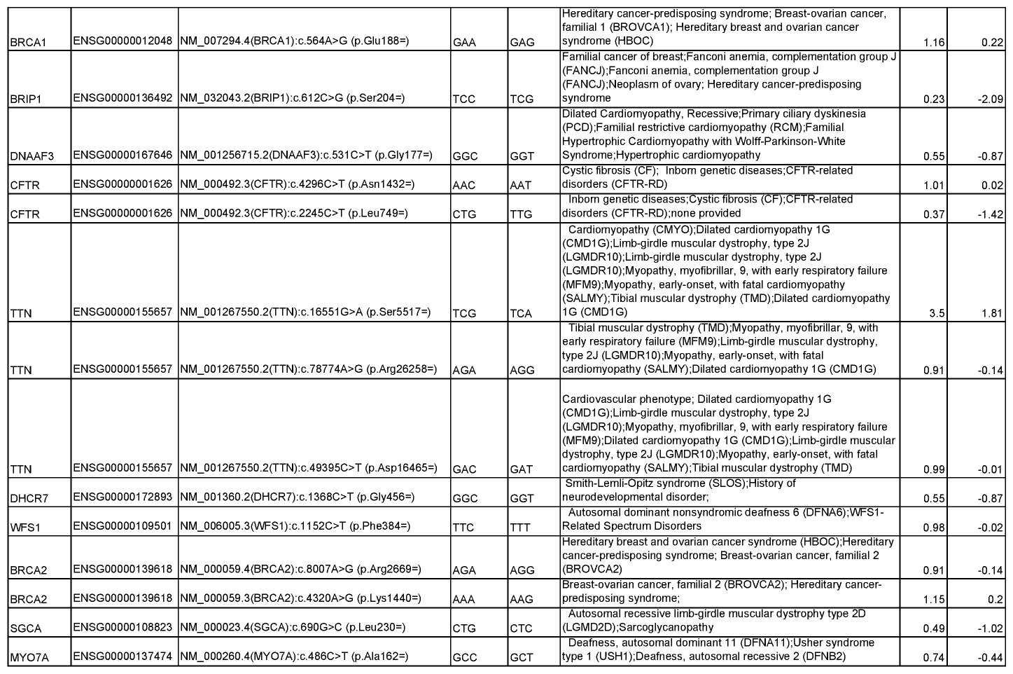

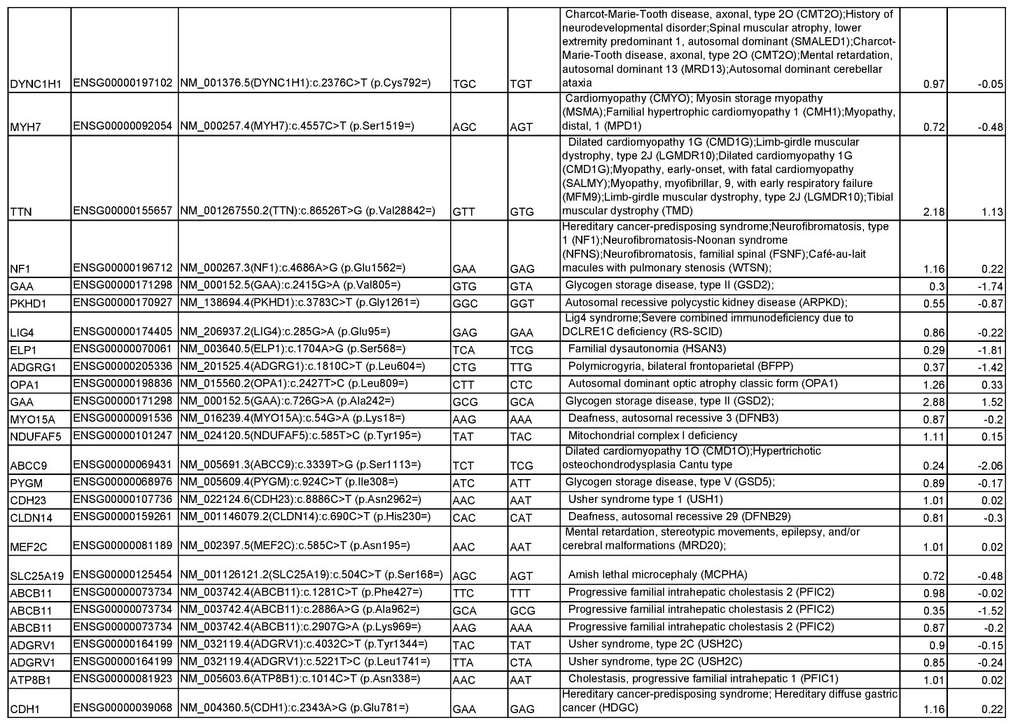

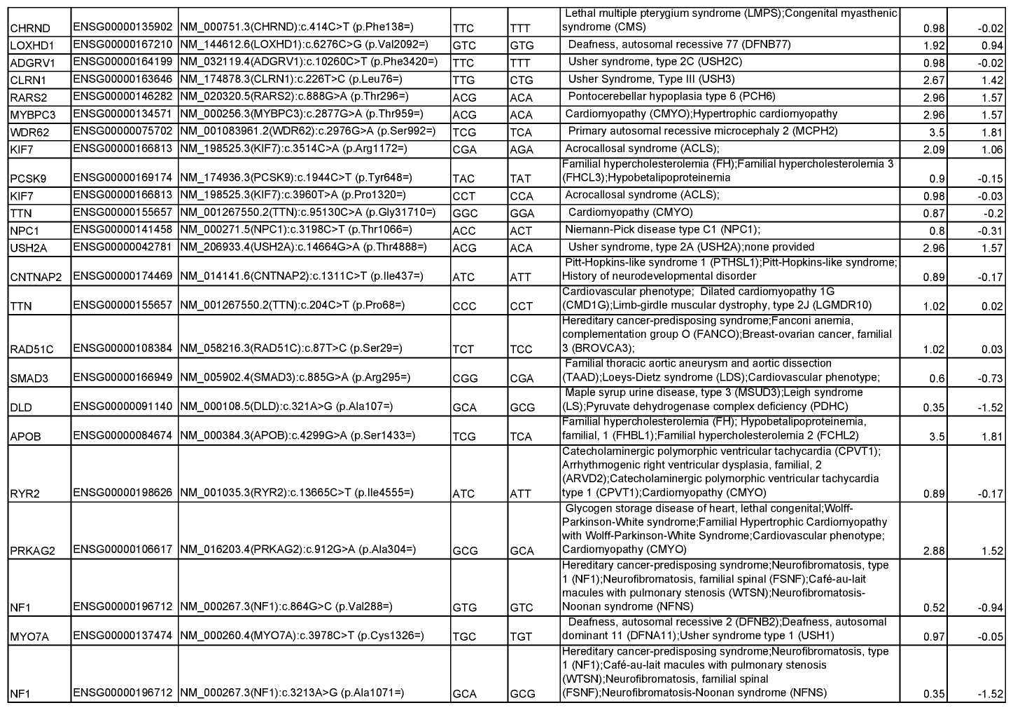

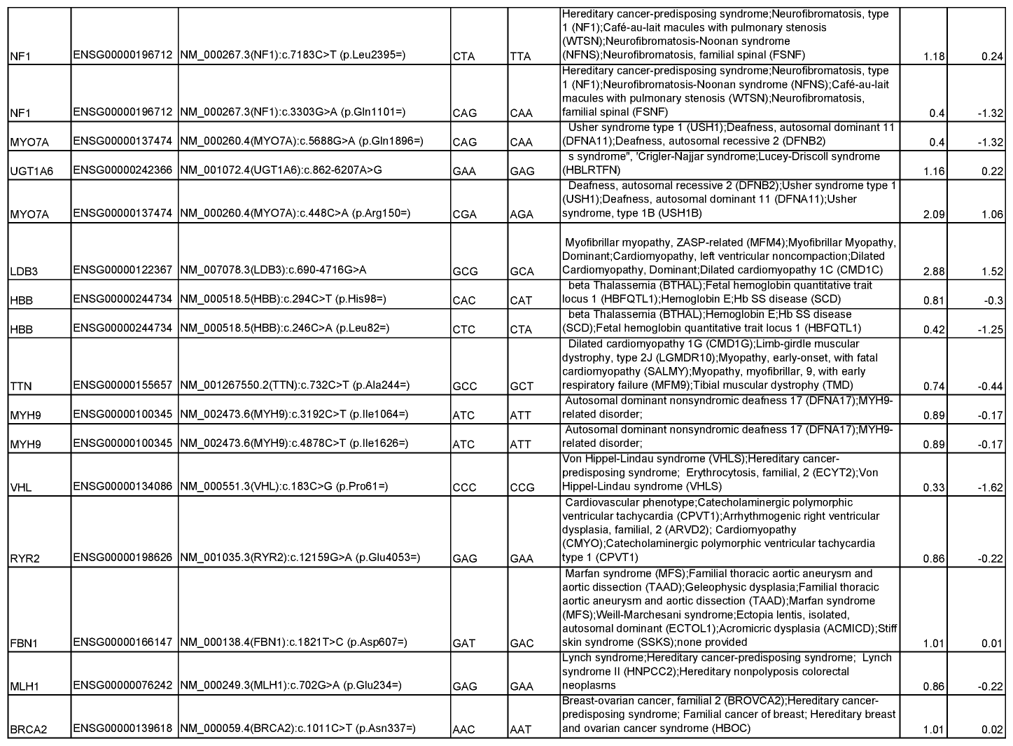

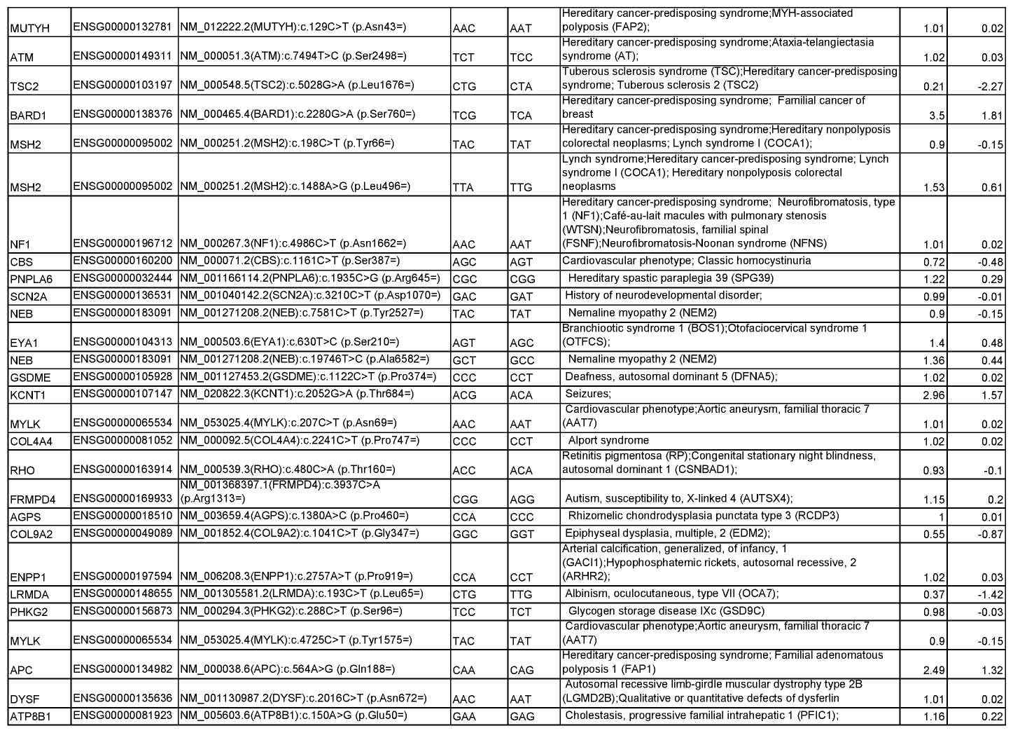

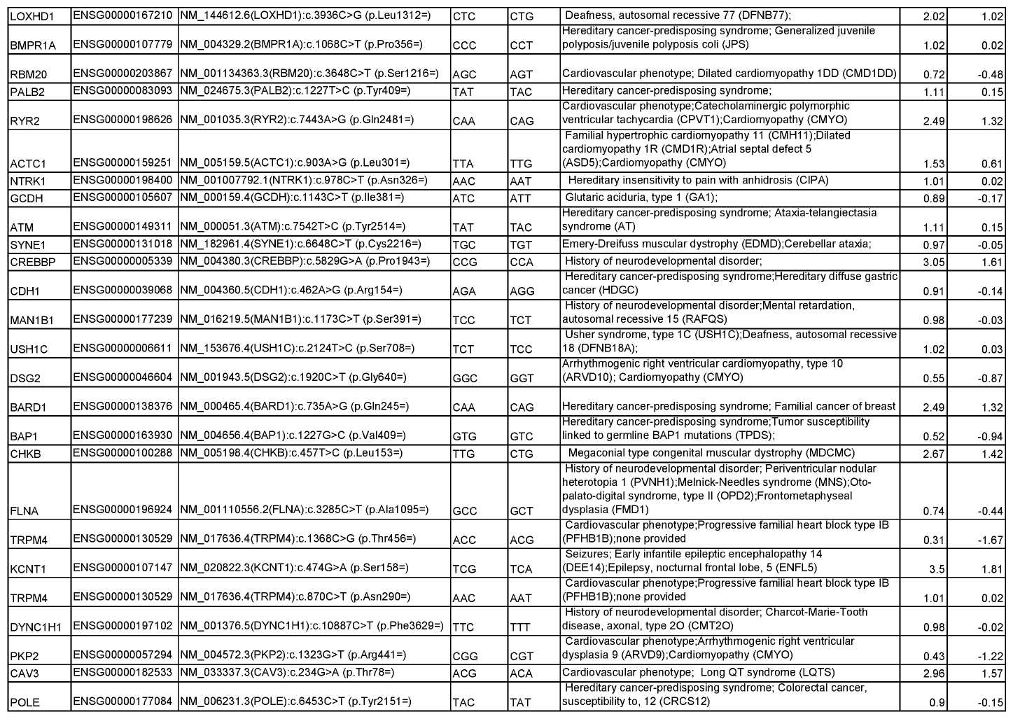

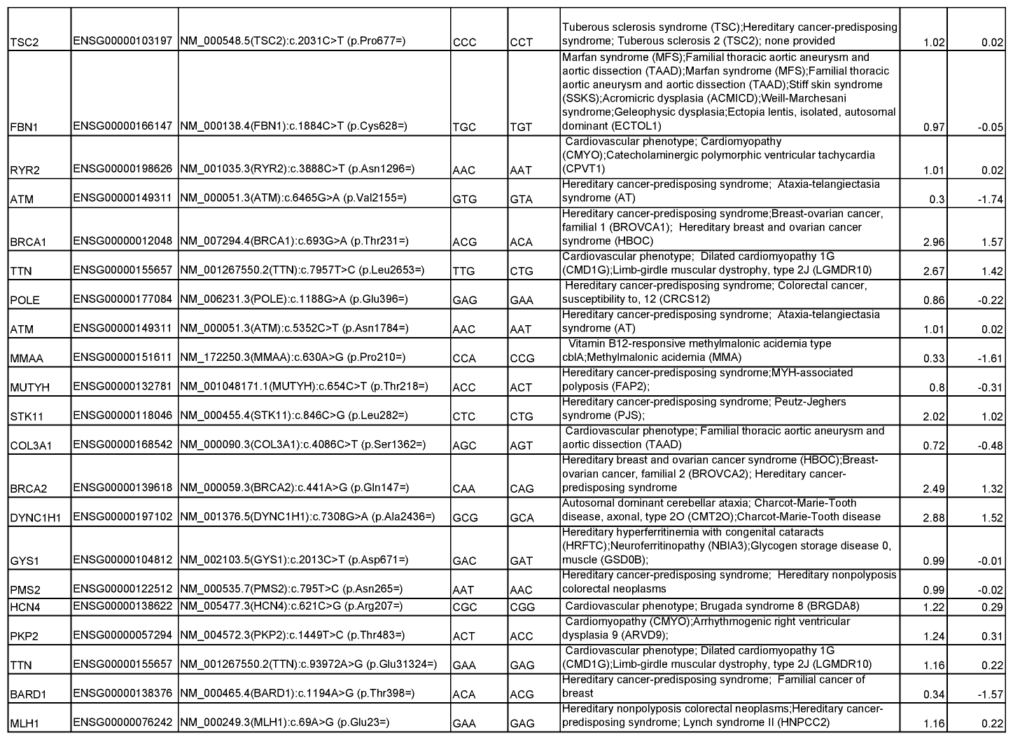

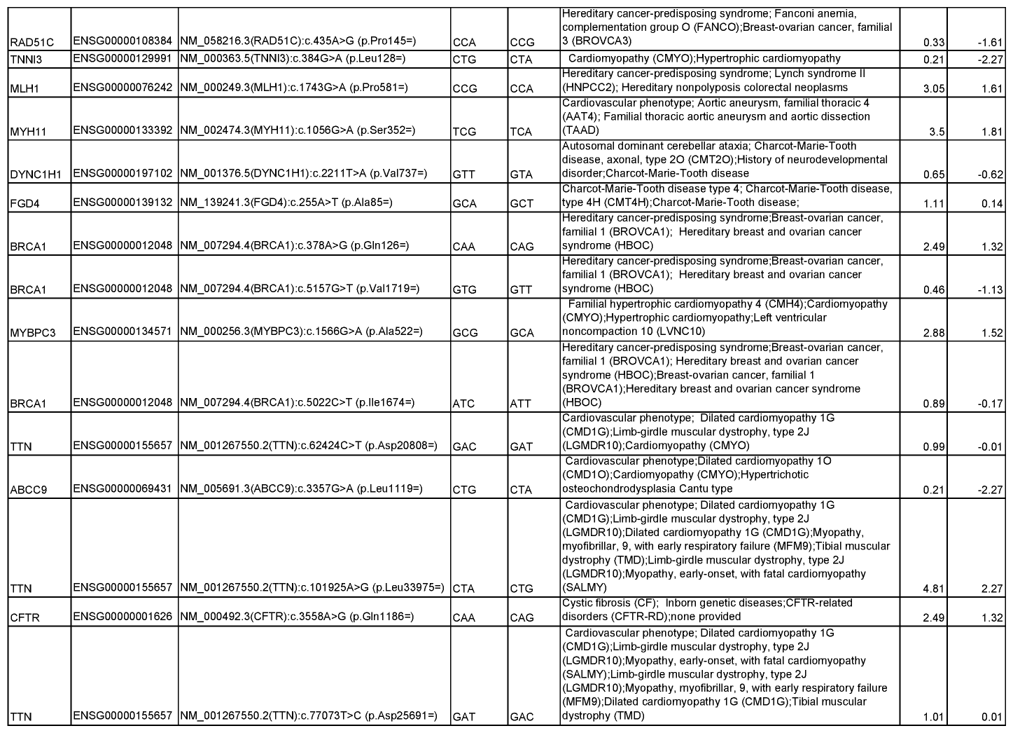

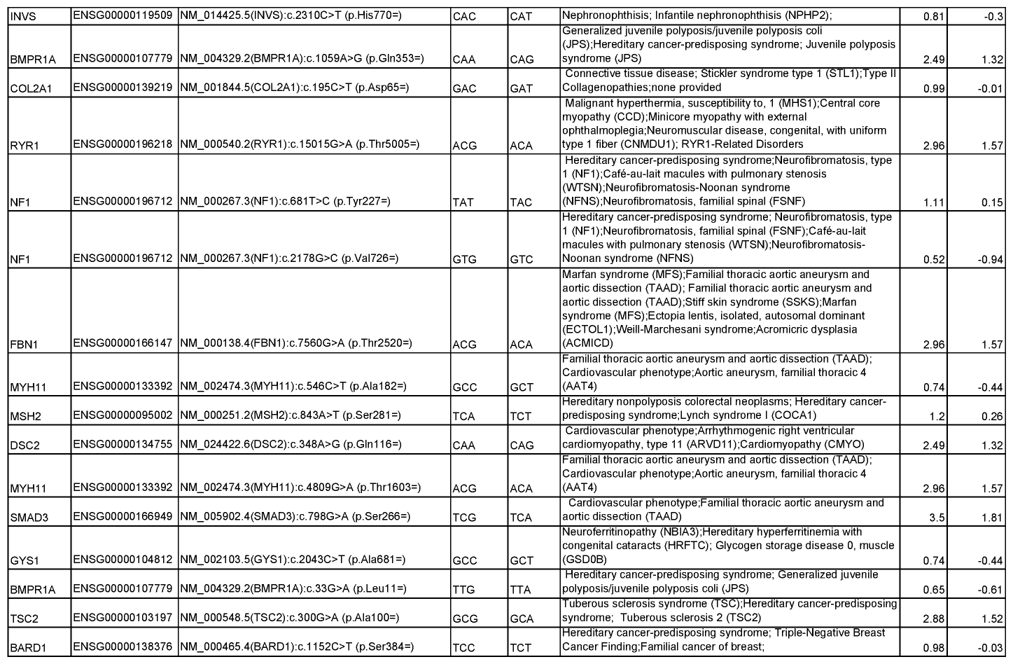

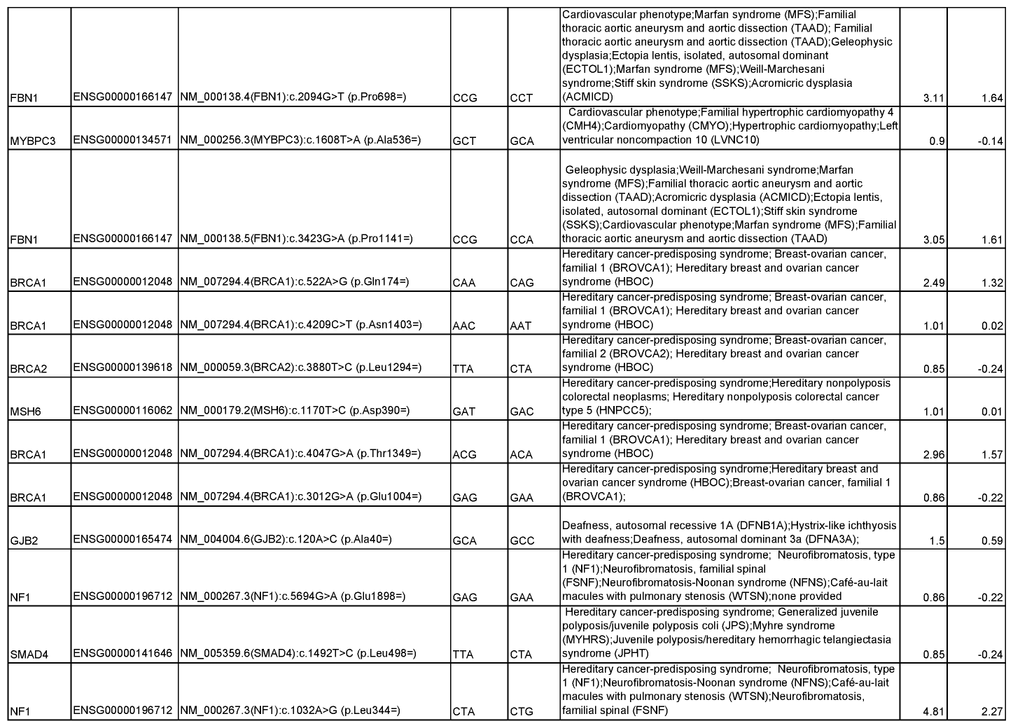

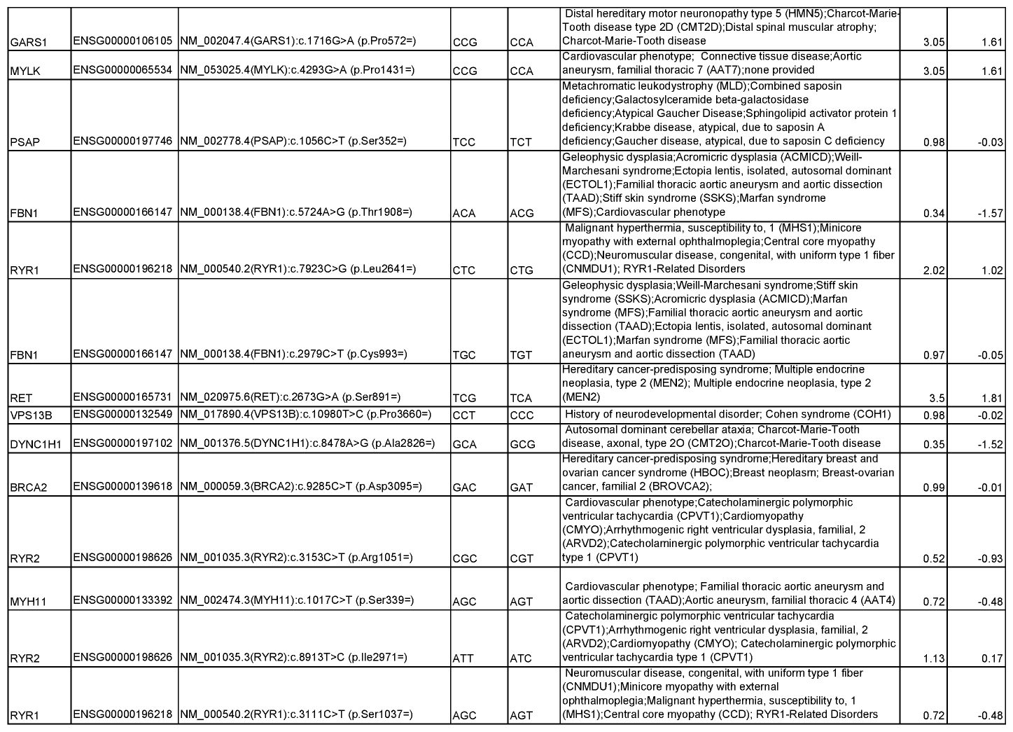

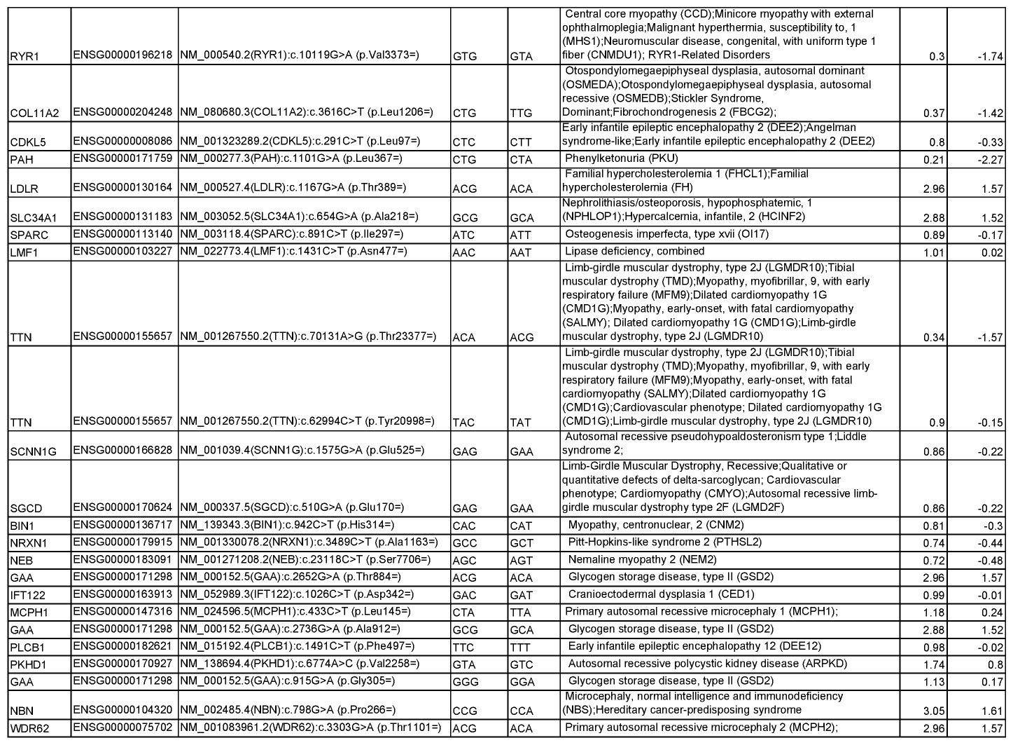

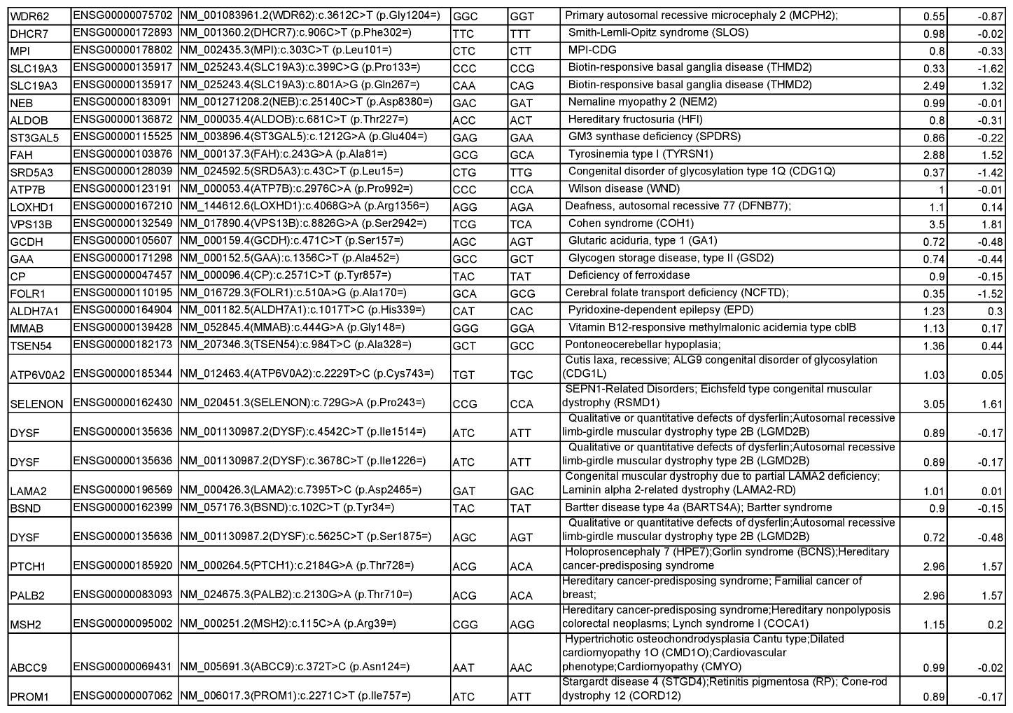

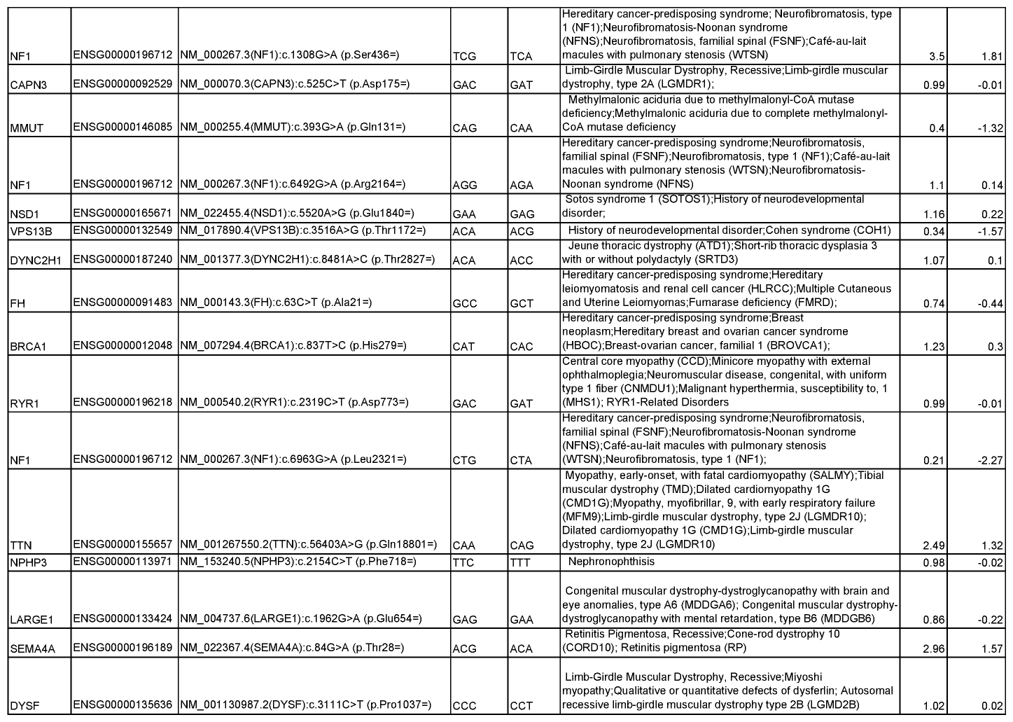

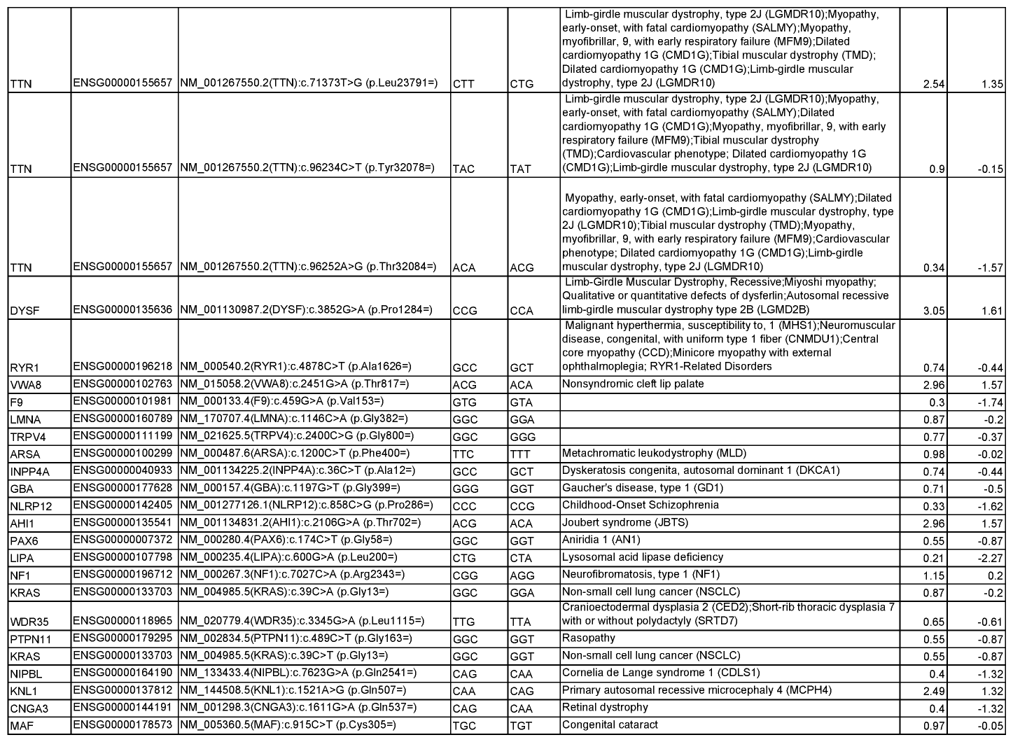

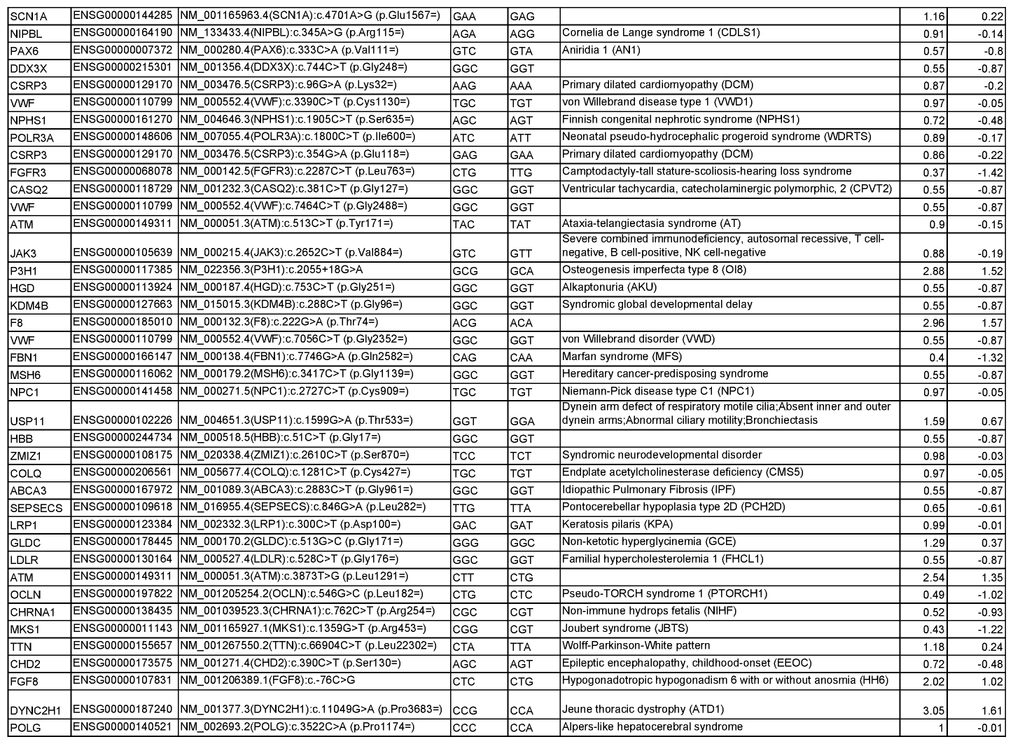

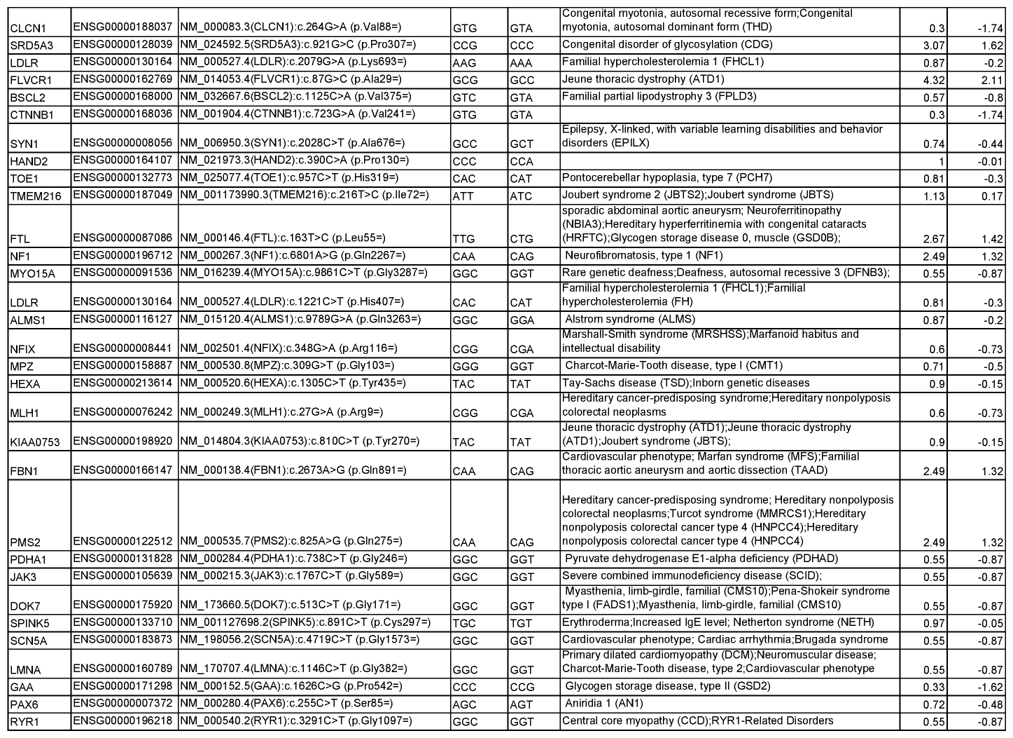

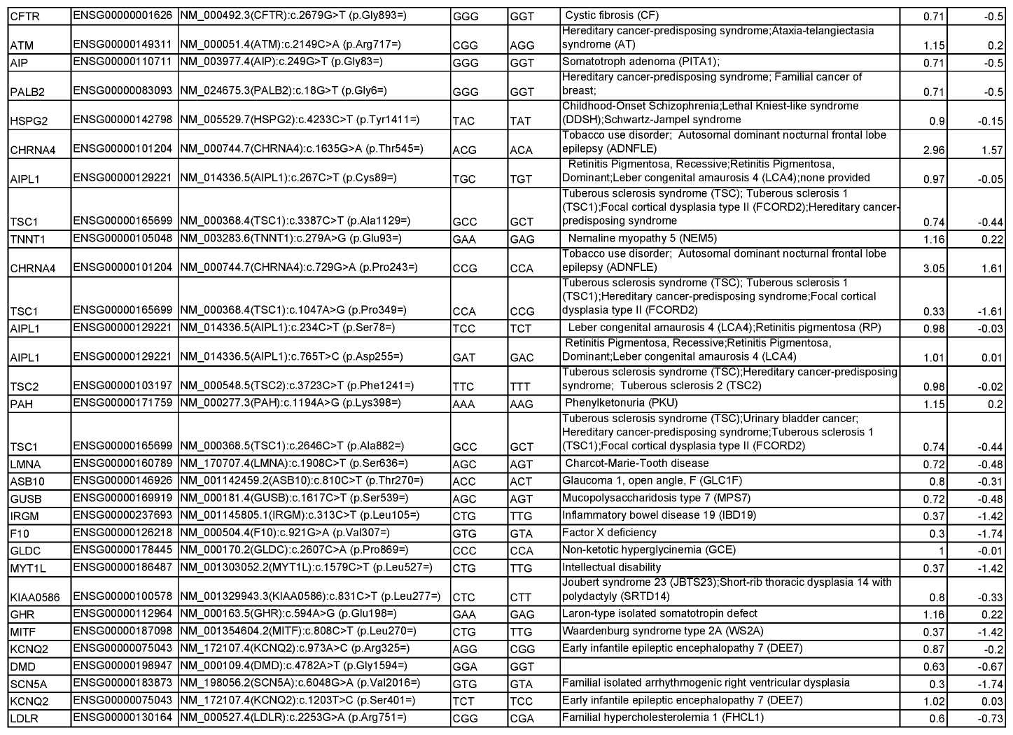

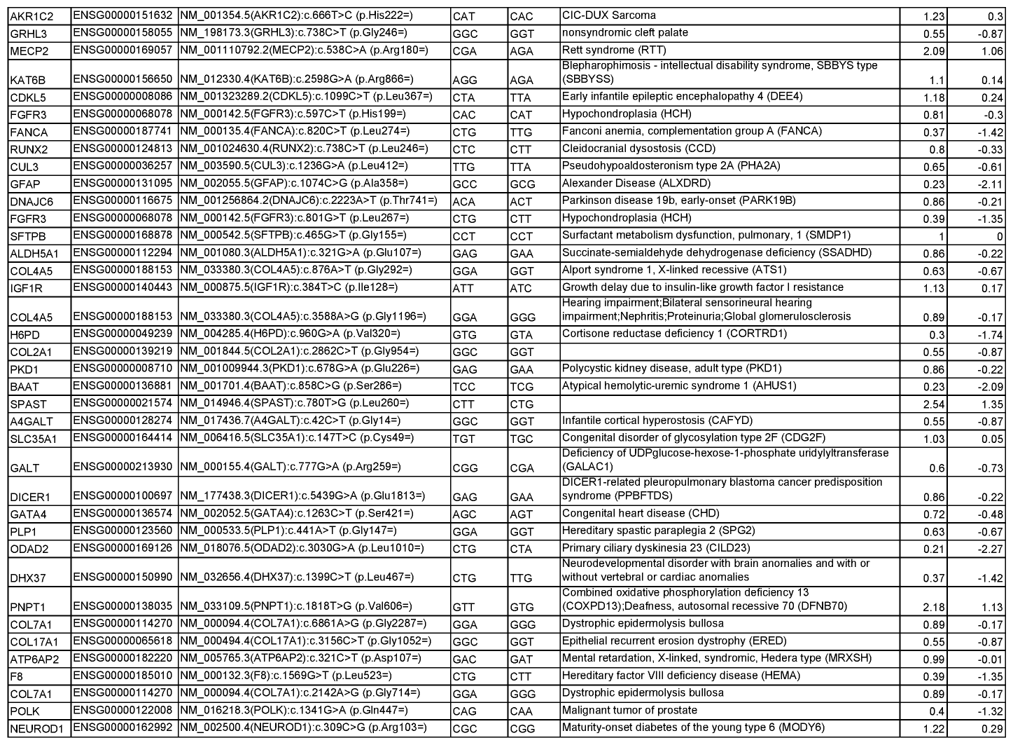

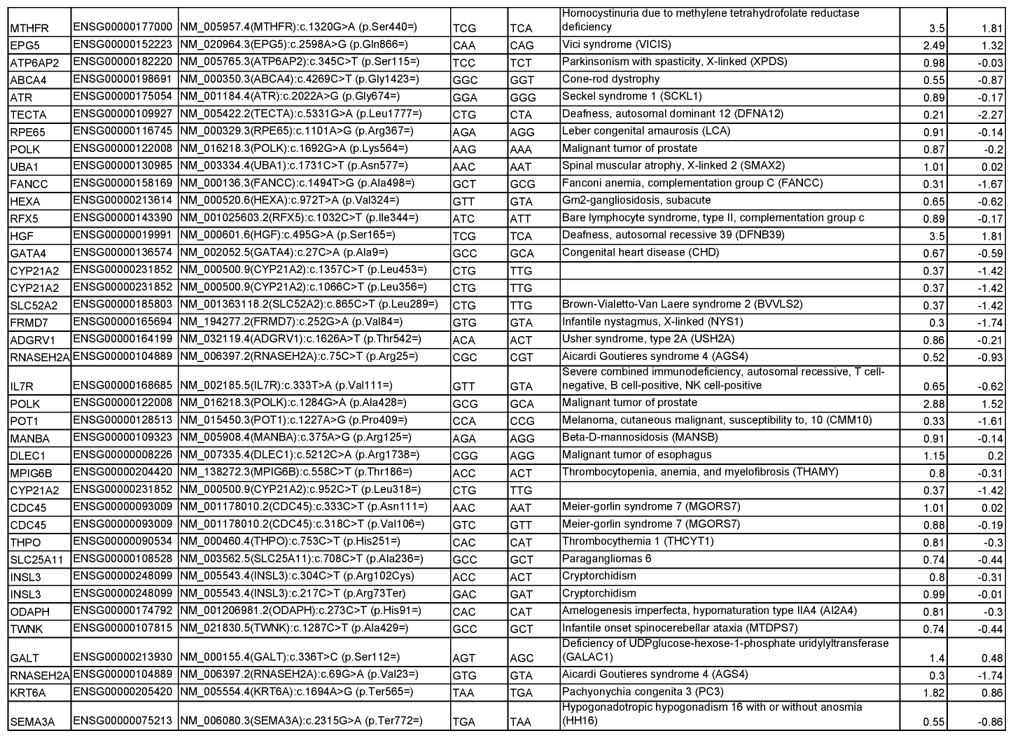

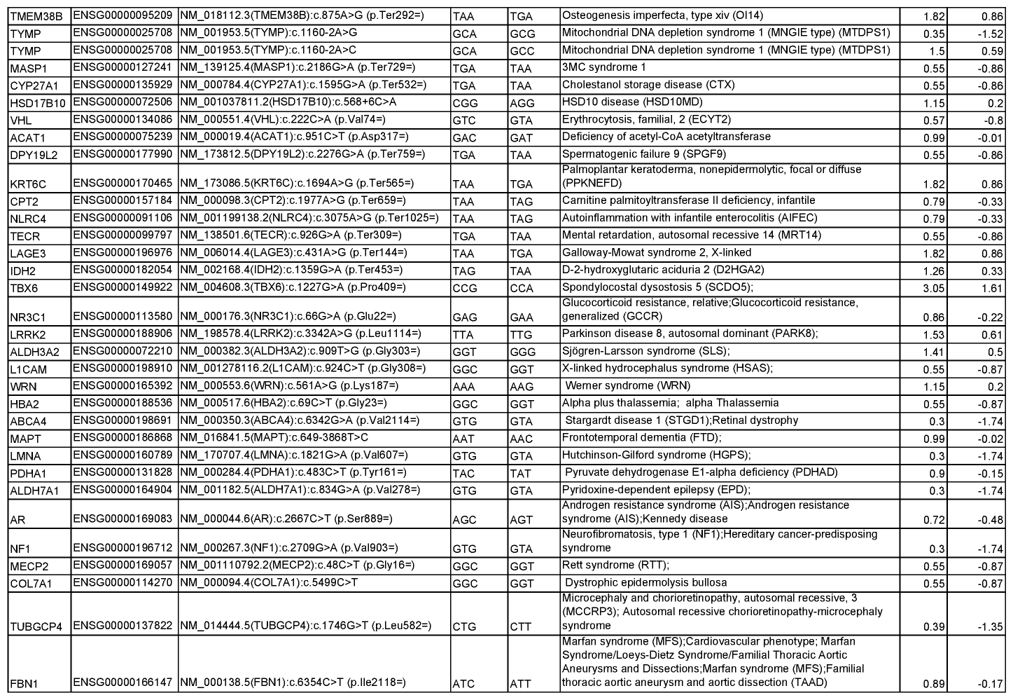

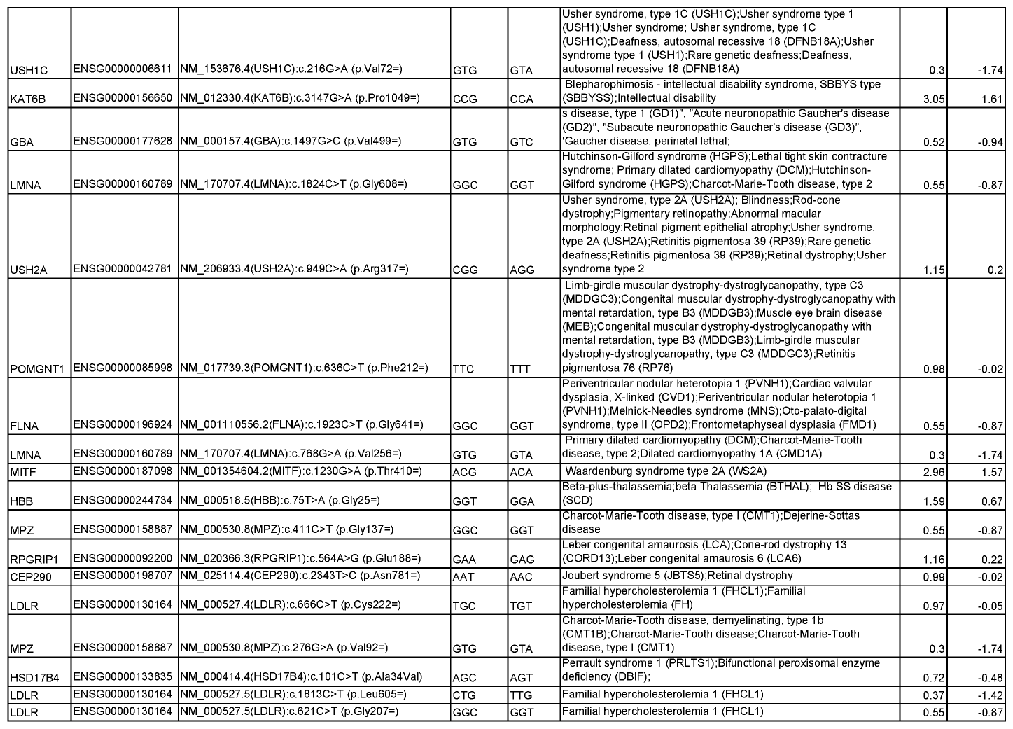

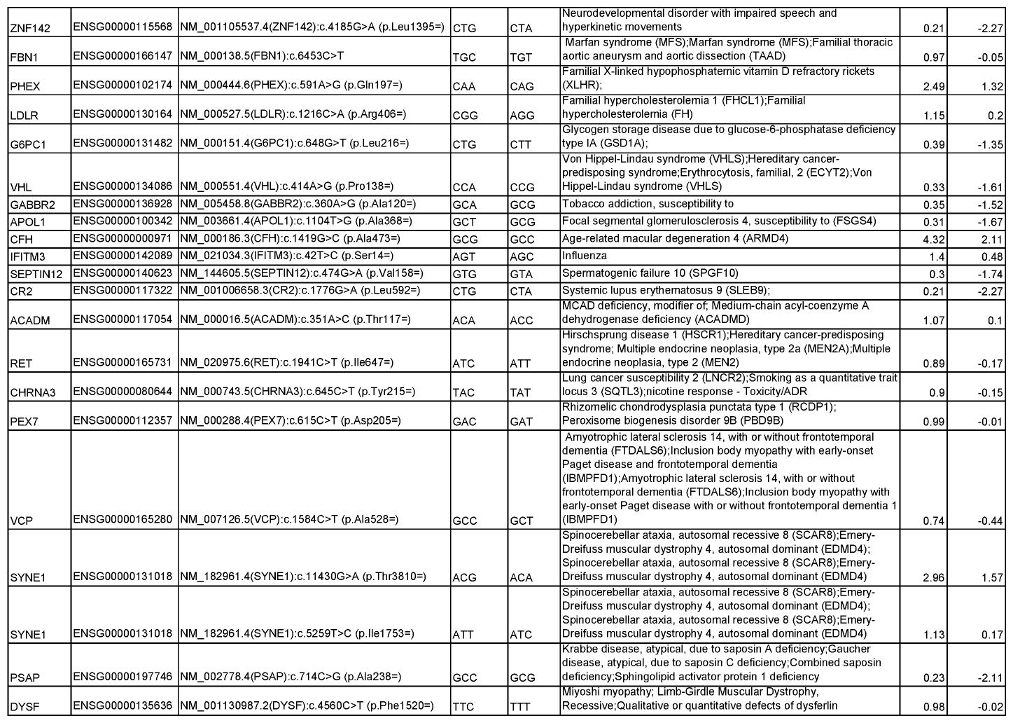

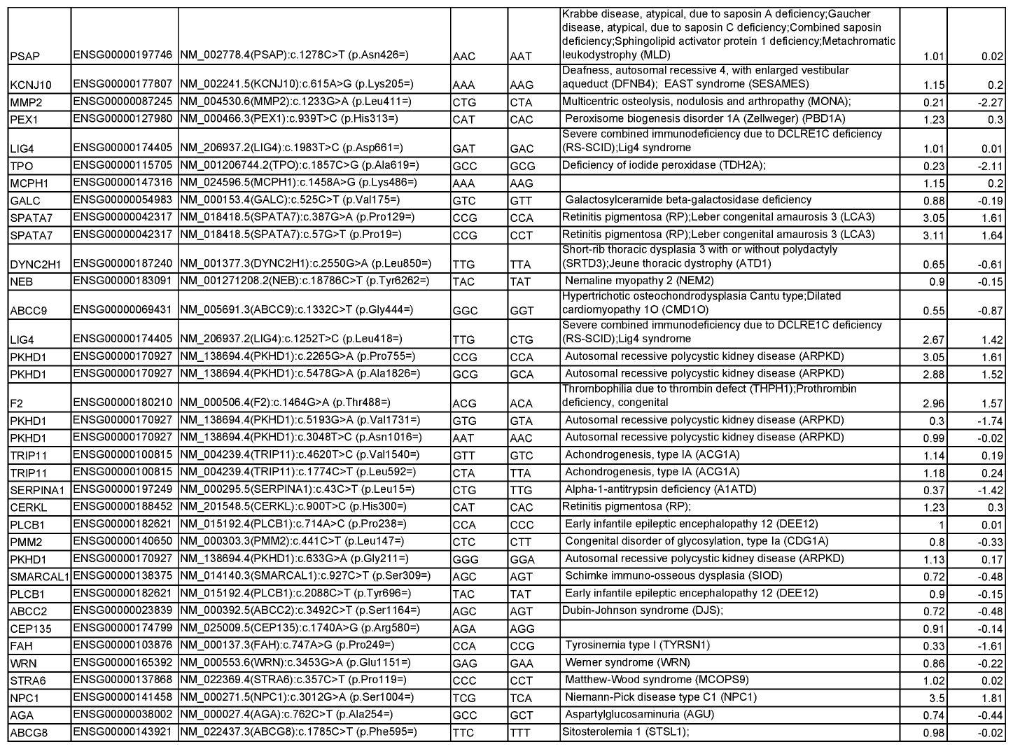

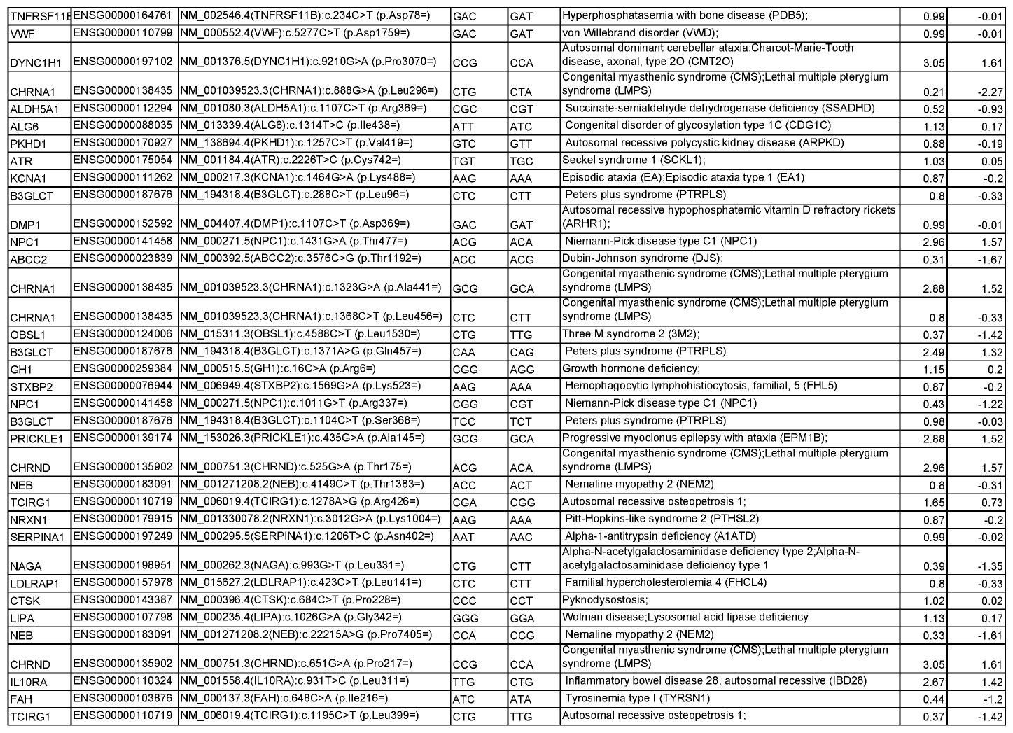

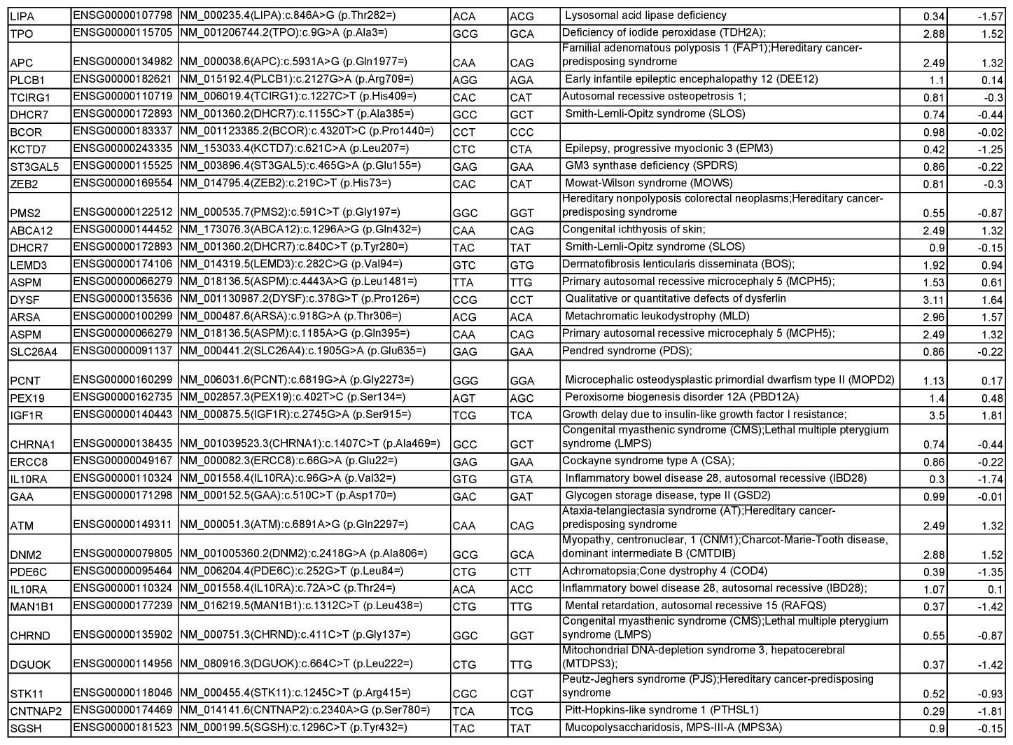

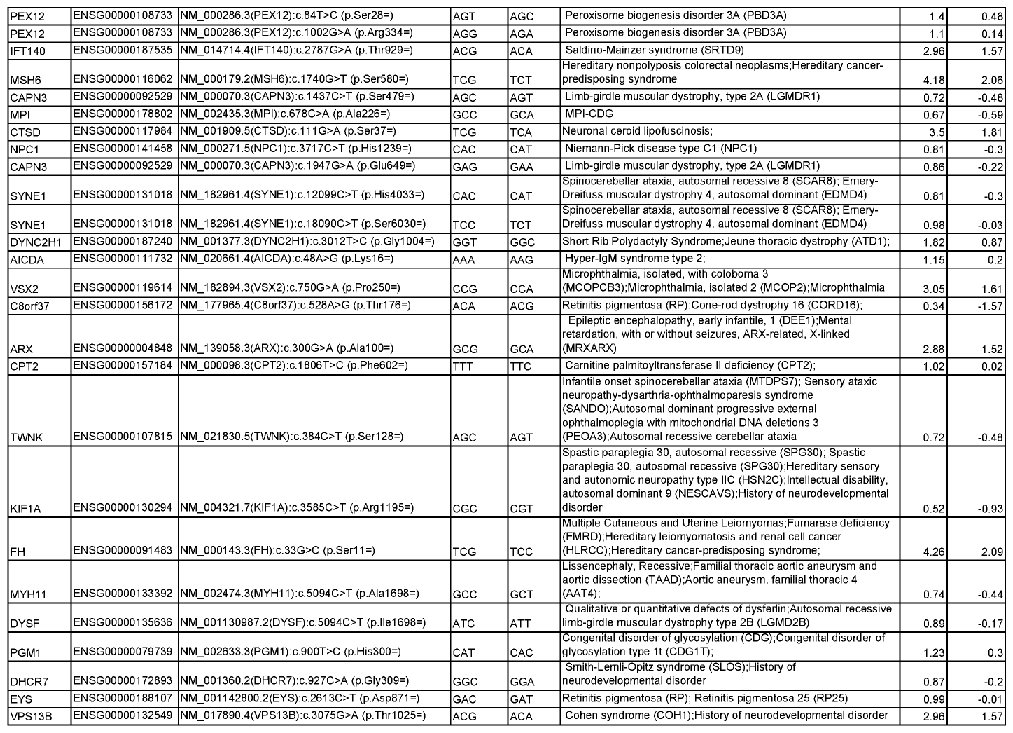

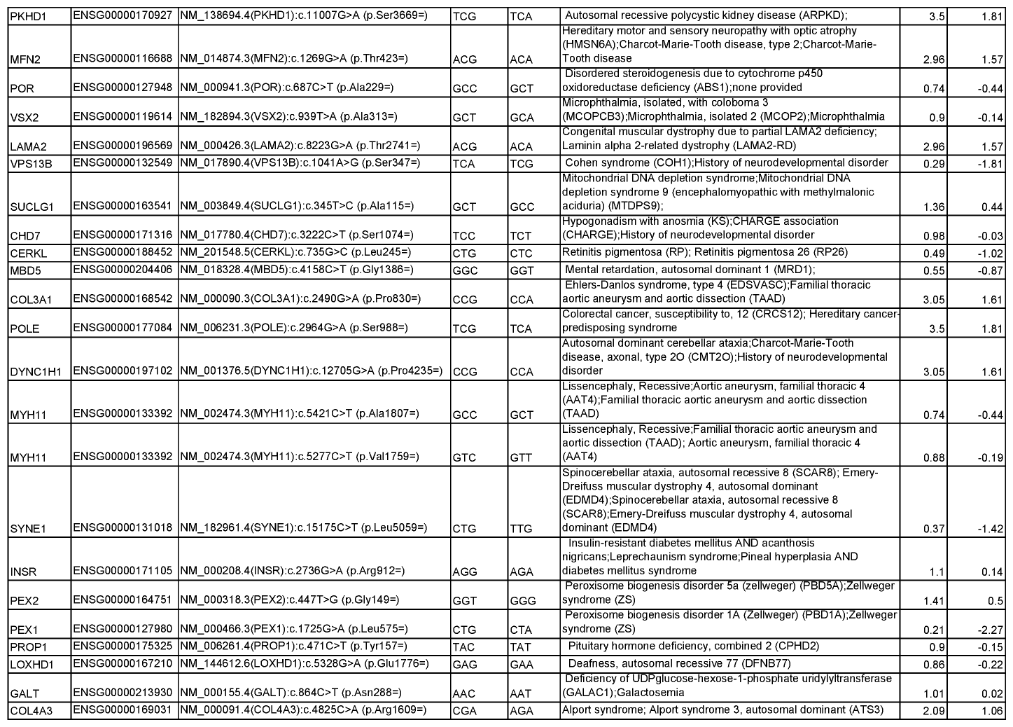

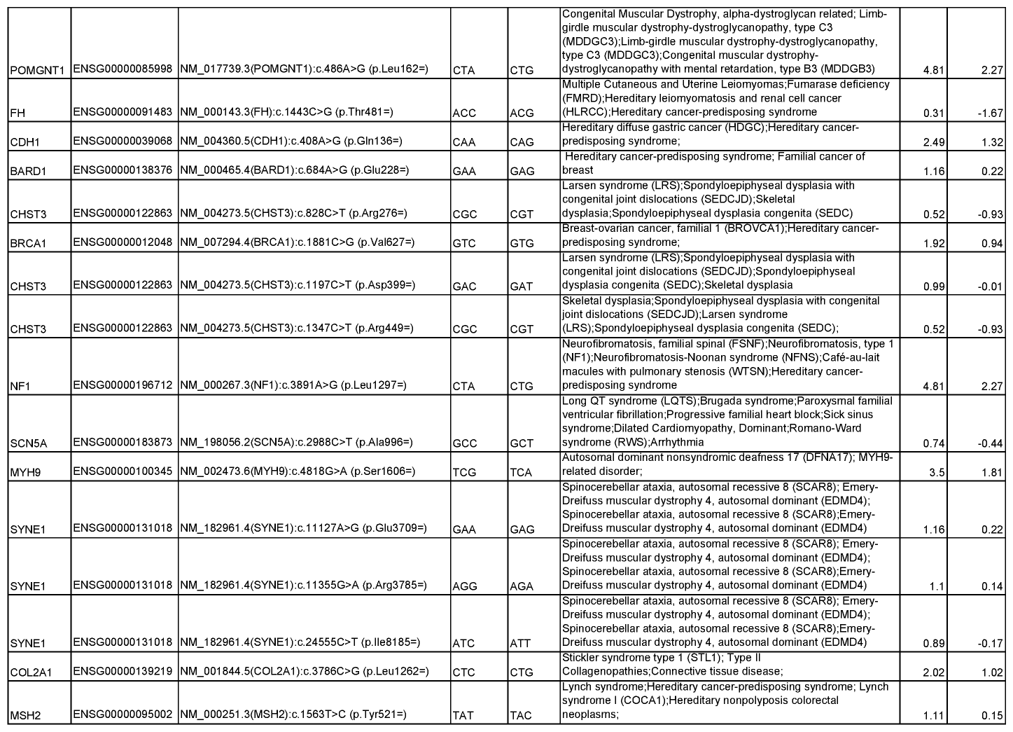

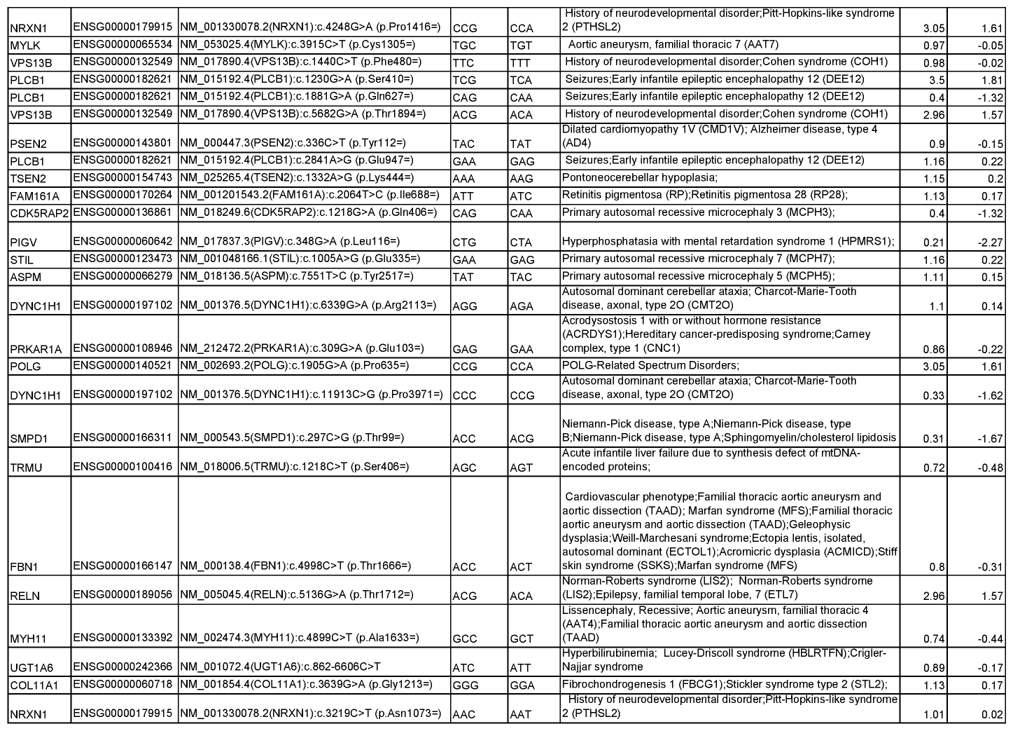

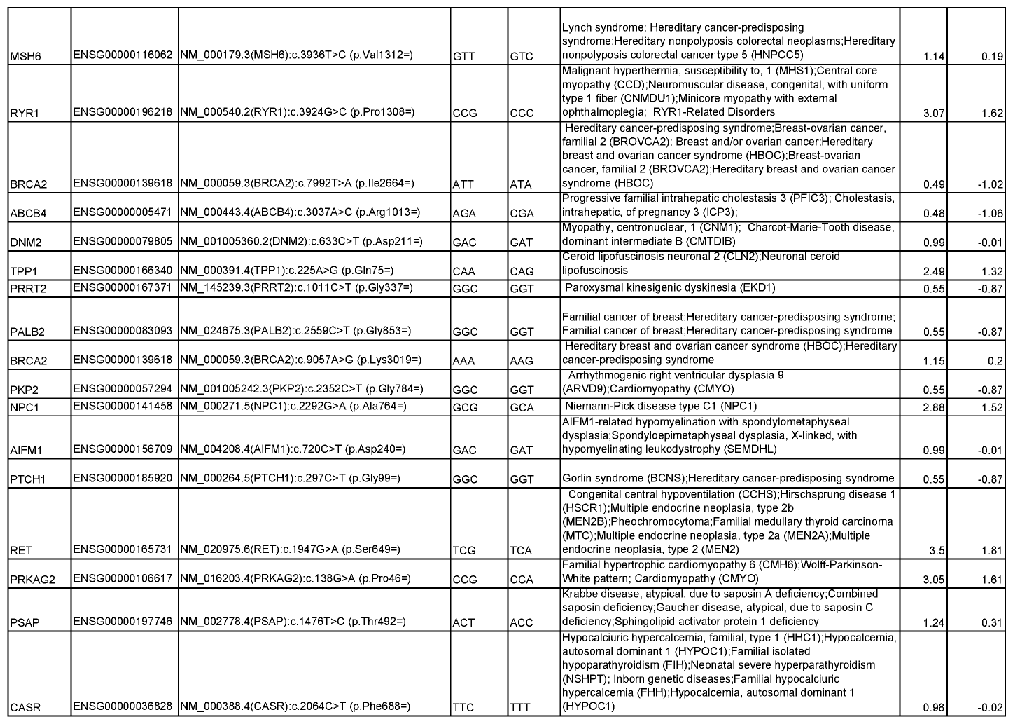

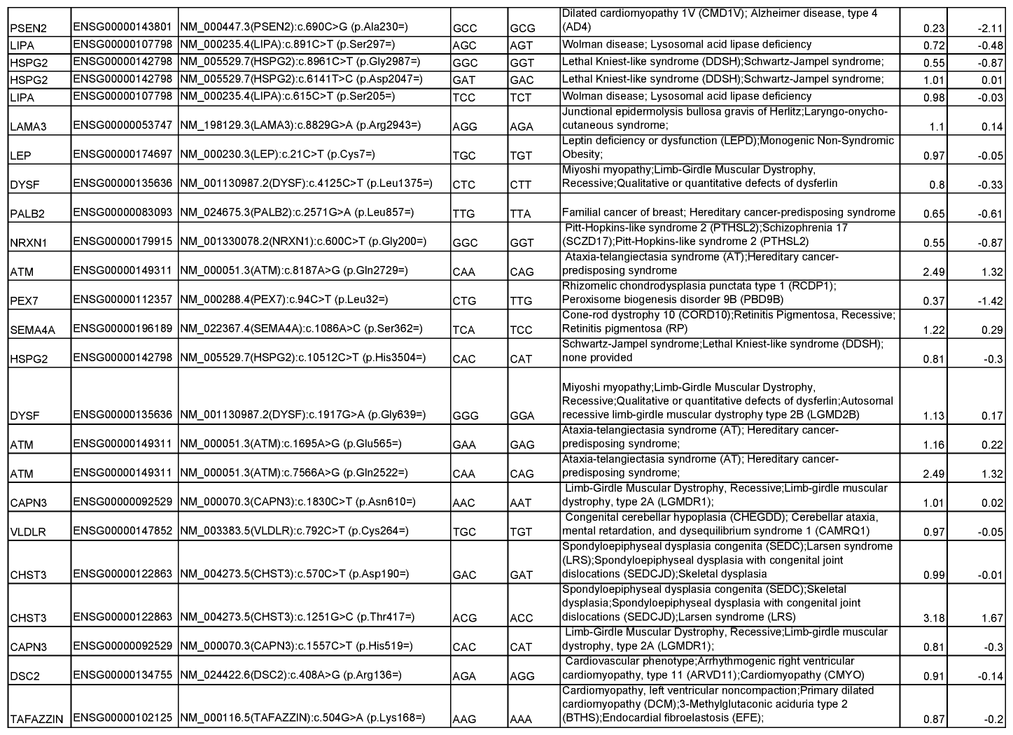

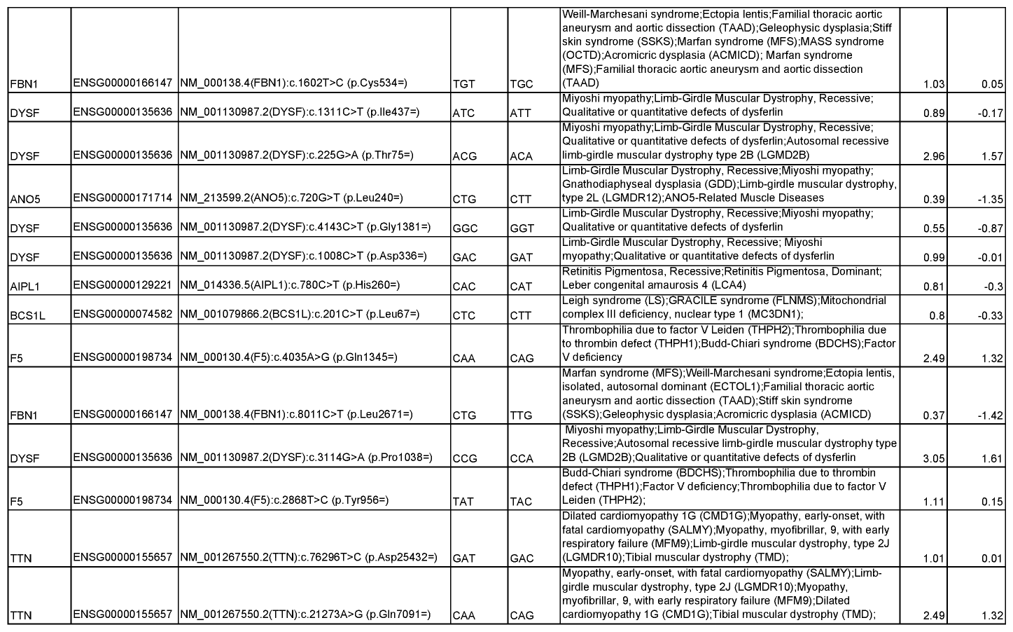

Exemplary synonymous SNPs and related genes are provided in Tables 1 and FIG. 4. In Table 1, the column with the heading “codon from/to” describes a wildtype codon for a particular transcript and the mutated codon. In FIG. 4, the column with the heading “original codon” describes a wildtype codon for a particular transcript, while the mutated codon is listed in the column entitled “new codon.” Information in these tables was derived from publicly available databases, such as MyVariant.info, MyGene.info, each of which is provided by The Scripps Research Institute; RefSeq (https://www.ncbi.nlm.nih.gov/refseq/); and CoCoPUTs (https://hive.biochemistry.gwu.edu/dna.cgi?cmd=tissue_codon_usage&id=586358&mode=cocop uts).

In an embodiment, a cell or subject described in a method of treatment, a method of modulating a tRNA pool, or a method of evaluation disclosed herein has a SNP provided in Table 1 or FIG. 4. In an embodiment, a cell or subject described in a method of treatment, a method of modulating a tRNA pool, or a method of evaluation disclosed herein has a disease listed in Table 1 or FIG. 4. In an embodiment, a cell or subject described in a method of treatment, a method of modulating a tRNA pool, or a method of evaluation disclosed herein has a SNP and the corresponding disease listed in Table 1 or FIG. 4.

TABLE 1
Exemplary SNPs and correlated diseases

| | | | | | Ratio of |
|---|---|---|---|---|---|
| | | | | | tRNA |
| | | | Codon | Anticodon | frequency |
| Disease Group | Disease | Transcript | from/To | from/to | (from/to) |
| Cardiovascular | Cardiac hypertrophy | ENST00000357596 | CTA/CTC | TAG/GAG | 19.3 |
| Cardiovascular | Cardiac hypertrophy | ENST00000357596 | CTA/CTG | TAG/CAG | 4.8 |
| Cardiovascular | Cardiac hypertrophy | ENST00000378319 | CTA/CTC | TAG/GAG | 19.3 |
| Cardiovascular | Cardiac hypertrophy | ENST00000378319 | CTA/CTG | TAG/CAG | 4.8 |
| Cardiovascular | Cardiac hypertrophy | ENST00000442195 | CTA/CTC | TAG/GAG | 19.3 |
| Cardiovascular | Cardiac hypertrophy | ENST00000442195 | CTA/CTG | TAG/CAG | 4.8 |
| Cardiovascular | Cardiac hypertrophy | ENST00000465045 | CTA/CTC | TAG/GAG | 19.3 |
| Cardiovascular | Cardiac hypertrophy | ENST00000465045 | CTA/CTG | TAG/CAG | 4.8 |
| Cardiovascular | Cardiac hypertrophy | ENST00000485919 | CTA/CTC | TAG/GAG | 19.3 |
| Cardiovascular | Cardiac hypertrophy | ENST00000485919 | CTA/CTG | TAG/CAG | 4.8 |
| Cardiovascular | Cardiac hypertrophy | ENST00000488958 | CTA/CTC | TAG/GAG | 19.3 |
| Cardiovascular | Cardiac hypertrophy | ENST00000488958 | CTA/CTG | TAG/CAG | 4.8 |
| Cardiovascular | Cardiac hypertrophy | ENST00000496623 | CTA/CTC | TAG/GAG | 19.3 |
| Cardiovascular | Cardiac hypertrophy | ENST00000496623 | CTA/CTG | TAG/CAG | 4.8 |
| Cardiovascular | Coronary artery disease | ENST00000375377 | CAC/CAT | GTG/ATG | 4.8 |
| Cardiovascular | Coronary artery disease | ENST00000375377 | CAC/CAT | GTG/ATG | 4.8 |
| Cardiovascular | Coronary artery disease | ENST00000413988 | GGC/GGT | GCC/ACC | 2235.1 |
| Cardiovascular | Hypertension | ENST00000229768 | GGA/GGG | TCC/CCC | 1.2 |
| Cardiovascular | Hypertension | ENST00000563726 | CCA/CCT | TGG/AGG | 16.7 |
| Cardiovascular | Hypertension | ENST00000563726 | CCA/CCG | TGG/CGG | 2.2 |
| Cardiovascular | Obesity-related traits | ENST00000318130 | ATC/ATT | GAT/AAT | 1443.7 |
| Cardiovascular | Obesity-related traits | ENST00000367512 | ATC/ATT | GAT/AAT | 1443.7 |
| Cardiovascular | Obesity-related traits | ENST00000439962 | ATC/ATT | GAT/AAT | 1443.7 |
| Cardiovascular | Type 1 diabetes | ENST00000261340 | AGC/AGT | GCT/ACT | 400.5 |
| Cardiovascular | Type 1 diabetes | ENST00000290855 | AGC/AGT | GCT/ACT | 400.5 |
| Cardiovascular | Type 1 diabetes | ENST00000325960 | AGC/AGT | GCT/ACT | 400.5 |
| Cardiovascular | Type 1 diabetes | ENST00000430909 | AGC/AGT | GCT/ACT | 400.5 |
| Cardiovascular | Type 1 diabetes | ENST00000444971 | AGC/AGT | GCT/ACT | 400.5 |
| Cardiovascular | Type 1 diabetes | ENST00000476198 | AGC/AGT | GCT/ACT | 400.5 |
| Cardiovascular | Type 1 diabetes | ENST00000479877 | AGC/AGT | GCT/ACT | 400.5 |
| Cardiovascular | Type 1 diabetes | ENST00000536355 | AGC/AGT | GCT/ACT | 400.5 |
| Cardiovascular | Type 1 diabetes | ENST00000543300 | AGC/AGT | GCT/ACT | 400.5 |
| Cardiovascular | Type 1 diabetes | ENST00000544322 | AGC/AGT | GCT/ACT | 400.5 |
| Cardiovascular | Type 1 diabetes | ENST00000263125 | CGA/AGA | TCG/TCT | 0.9 |
| Cardiovascular | Type 1 diabetes | ENST00000397176 | CGA/AGA | TCG/TCT | 0.9 |
| Cardiovascular | Type 1 diabetes | ENST00000539722 | CGA/AGA | TCG/TCT | 0.9 |
| Cardiovascular | Type 1 diabetes | ENST00000610727 | CGA/AGA | TCG/TCT | 0.9 |
| Cardiovascular | Type 2 diabetes | ENST00000226760 | AAC/AAT | GTT/ATT | 655.6 |
| Cardiovascular | Type 2 diabetes | ENST00000503569 | AAC/AAT | GTT/ATT | 655.6 |
| Dermatology | Psoriasis | ENST00000289431 | TGC/TGT | GCA/ACA | 111.3 |
| Dermatology | Psoriasis | ENST00000422556 | TGC/TGT | GCA/ACA | 111.3 |
| Endocrine | Endometriosis | ENST00000381434 | TAC/TAT | GTA/ATA | 268.1 |
| Endocrine | Endometriosis | ENST00000417746 | TAC/TAT | GTA/ATA | 268.1 |
| Endocrine | Endometriosis | ENST00000456383 | TAC/TAT | GTA/ATA | 268.1 |
| Endocrine | Endometriosis | ENST00000611532 | TAC/TAT | GTA/ATA | 268.1 |
| Immunology | Chronic inflammatory | ENST00000171111 | GAC/GAT | GTC/ATC | 238.2 |
| | diseases (ankylosing | | | | |
| | spondylitis, Crohn's | | | | |
| | disease, psoriasis, primary | | | | |
| | sclerosing cholangitis, | | | | |
| | ulcerative colitis) | | | | |
| | (pleiotropy) | | | | |
| Immunology | Chronic inflammatory | ENST00000393623 | GAC/GAT | GTC/ATC | 238.2 |
| | diseases (ankylosing | | | | |
| | spondylitis, Crohn's | | | | |
| | disease, psoriasis, primary | | | | |
| | sclerosing cholangitis, | | | | |
| | ulcerative colitis) | | | | |
| | (pleiotropy) | | | | |
| Immunology | Chronic inflammatory | ENST00000592478 | GAC/GAT | GTC/ATC | 238.2 |
| | diseases (ankylosing | | | | |
| | spondylitis, Crohn's | | | | |
| | disease, psoriasis, primary | | | | |
| | sclerosing cholangitis, | | | | |
| | ulcerative colitis) | | | | |
| | (pleiotropy) | | | | |
| Immunology | Crohn's disease | ENST00000300590 | ATC/ATT | GAT/AAT | 1443.7 |
| Immunology | Crohn's disease | ENST00000330943 | ATC/ATT | GAT/AAT | 1443.7 |
| Immunology | Crohn's disease | ENST00000423026 | ATC/ATT | GAT/AAT | 1443.7 |
| Immunology | Crohn's disease | ENST00000568993 | ATC/ATT | GAT/AAT | 1443.7 |
| Immunology | Crohn's disease | ENST00000610485 | ATC/ATT | GAT/AAT | 1443.7 |
| Immunology | Graves' disease | ENST00000561890 | CAC/CAT | GTG/ATG | 4.8 |
| Neurology | Alzheimer disease and age | ENST00000356711 | AAG/AAA | CTT/TTT | 2.1 |
| | of onset | | | | |
| Neurology | Alzheimer disease and age | ENST00000357550 | AAG/AAA | CTT/TTT | 2.1 |
| | of onset | | | | |
| Neurology | Alzheimer disease and age | ENST00000457511 | AAG/AAA | CTT/TTT | 2.1 |
| | of onset | | | | |
| Neurology | Alzheimer's disease or | ENST00000198536 | AGG/CGG | CCT/CCG | 1.3 |
| | family history of | | | | |
| | Alzheimer's disease | | | | |
| Neurology | Alzheimer's disease or | ENST00000350573 | AGG/CGG | CCT/CCG | 1.3 |
| | family history of | | | | |
| | Alzheimer's disease | | | | |
| Neurology | Alzheimer's disease or | ENST00000394000 | AGG/CGG | CCT/CCG | 1.3 |
| | family history of | | | | |
| | Alzheimer's disease | | | | |
| Neurology | Alzheimer's disease or | ENST00000432297 | AGG/CGG | CCT/CCG | 1.3 |
| | family history of | | | | |
| | Alzheimer's disease | | | | |
| Neurology | Alzheimer's disease or | ENST00000453419 | AGG/CGG | CCT/CCG | 1.3 |
| | family history of | | | | |
| | Alzheimer's disease | | | | |
| Neurology | Major depressive disorder | ENST00000292591 | CTG/TTG | CAG/CAA | 1.4 |
| Neurology | Major depressive disorder | ENST00000337755 | CTG/TTG | CAG/CAA | 1.4 |
| Neurology | Major depressive disorder | ENST00000519836 | CTG/TTG | CAG/CAA | 1.4 |
| Neurology | Major depressive disorder | ENST00000520918 | CTG/TTG | CAG/CAA | 1.4 |
| Neurology | Major depressive disorder | ENST00000373352 | TGC/TGT | GCA/ACA | 111.3 |
| Neurology | Major depressive disorder | ENST00000373371 | TGC/TGT | GCA/ACA | 111.3 |
| Neurology | Major depressive disorder | ENST00000430147 | TGC/TGT | GCA/ACA | 111.3 |
| Neurology | Major depressive disorder | ENST00000451404 | TGC/TGT | GCA/ACA | 111.3 |
| Neurology | Major depressive disorder | ENST00000610552 | TGC/TGT | GCA/ACA | 111.3 |
| Neurology | Migraine | ENST00000348159 | AAC/AAT | GTT/ATT | 655.6 |
| Neurology | Migraine | ENST00000360595 | AAC/AAT | GTT/ATT | 655.6 |
| Neurology | Migraine | ENST00000368240 | AAC/AAT | GTT/ATT | 655.6 |
| Neurology | Migraine | ENST00000475587 | AAC/AAT | GTT/ATT | 655.6 |
| Neurology | Parkinson's disease | ENST00000262407 | GTC/GTT | GAC/AAC | 725.5 |
| Neurology | Parkinson's disease | ENST00000587295 | GTC/GTT | GAC/AAC | 725.5 |
| Neurology | Parkinson's disease | ENST00000648408 | GTC/GTT | GAC/AAC | 725.5 |
| Neurology | Parkinson's disease | ENST00000398238 | AAG/AAA | CTT/TTT | 2.1 |
| Neurology | Parkinson's disease | ENST00000465370 | AAG/AAA | CTT/TTT | 2.1 |
| Neurology | Parkinson's disease | ENST00000575068 | AAG/AAA | CTT/TTT | 2.1 |
| Neurology | Parkinson's disease | ENST00000576346 | AAG/AAA | CTT/TTT | 2.1 |
| Neurology | Schizophrenia | ENST00000304743 | CTG/CTC | CAG/GAG | 4.0 |
| Neurology | Schizophrenia | ENST00000439984 | CTG/CTC | CAG/GAG | 4.0 |
| Oncology | Adverse response to | ENST00000282849 | GTG/GTA | CAC/TAC | 5.2 |
| | chemotherapy | | | | |
| | (neutropenia/leucopenia) | | | | |
| | (all antimicrotubule drugs) | | | | |
| Oncology | Breast cancer | ENST00000360962 | GTG/GTC | CAC/GAC | 1.2 |
| Oncology | Breast cancer | ENST00000525449 | GTG/GTC | CAC/GAC | 1.2 |
| Oncology | Breast cancer | ENST00000525908 | GTG/GTC | CAC/GAC | 1.2 |
| Oncology | Breast cancer | ENST00000527634 | GTG/GTC | CAC/GAC | 1.2 |
| Oncology | Breast cancer | ENST00000532565 | GTG/GTC | CAC/GAC | 1.2 |
| Oncology | Breast cancer | ENST00000540737 | GTG/GTC | CAC/GAC | 1.2 |
| Oncology | Breast cancer | ENST00000642265 | GTG/GTC | CAC/GAC | 1.2 |
| Oncology | Breast cancer | ENST00000359435 | AAG/AAA | CTT/TTT | 2.1 |
| Oncology | Breast cancer | ENST00000447614 | AAG/AAA | CTT/TTT | 2.1 |
| Oncology | Breast cancer | ENST00000595632 | AAG/AAA | CTT/TTT | 2.1 |
| Oncology | Breast cancer | ENST00000598188 | AAG/AAA | CTT/TTT | 2.1 |
| Oncology | Breast cancer | ENST00000601043 | AAG/AAA | CTT/TTT | 2.1 |
| Oncology | Breast cancer (early onset) | ENST00000399503 | ACC/ACT | GGT/AGT | 1099.1 |
| Oncology | Breast cancer (early onset) | ENST00000399503 | ACC/ACT | GGT/AGT | 1099.1 |
| Oncology | Carboplatin disposition in | ENST00000370449 | CAC/CAT | GTG/ATG | 4.8 |
| | epthelial ovarian cancer | | | | |
| Oncology | Carboplatin disposition in | ENST00000647814 | CAC/CAT | GTG/ATG | 4.8 |
| | epthelial ovarian cancer | | | | |
| Oncology | Clostridium difficile | ENST00000335385 | CTC/CTT | GAG/AAG | 90.2 |
| | infection in multiple | | | | |
| | myeloma | | | | |
| Oncology | Colorectal cancer | ENST00000349830 | CCC/CCG | GGG/CGG | 3.4 |
| Oncology | Colorectal cancer | ENST00000349830 | CCC/CCT | GGG/AGG | 25.7 |
| Oncology | Colorectal cancer | ENST00000398564 | CCC/CCG | GGG/CGG | 3.4 |
| Oncology | Colorectal cancer | ENST00000398564 | CCC/CCT | GGG/AGG | 25.7 |
| Oncology | Colorectal cancer | ENST00000518288 | CCC/CCG | GGG/CGG | 3.4 |
| Oncology | Colorectal cancer | ENST00000518288 | CCC/CCT | GGG/AGG | 25.7 |
| Oncology | Colorectal cancer | ENST00000520359 | CCC/CCG | GGG/CGG | 3.4 |
| Oncology | Colorectal cancer | ENST00000520359 | CCC/CCT | GGG/AGG | 25.7 |
| Oncology | Colorectal cancer | ENST00000522435 | CCC/CCG | GGG/CGG | 3.4 |
| Oncology | Colorectal cancer | ENST00000522435 | CCC/CCT | GGG/AGG | 25.7 |
| Oncology | Endometrial cancer | ENST00000247270 | AGC/AGT | GCT/ACT | 400.5 |
| | ((endometrioid histology) | | | | |
| Oncology | Esophageal squamous cell | ENST00000269187 | CTG/TTG | CAG/CAA | 1.4 |
| | cancer (length of survival) | | | | |
| Oncology | Esophageal squamous cell | ENST00000440549 | CTG/TTG | CAG/CAA | 1.4 |
| | cancer (length of survival) | | | | |
| Oncology | Esophageal squamous cell | ENST00000586829 | CTG/TTG | CAG/CAA | 1.4 |
| | cancer (length of survival) | | | | |
| Oncology | Esophageal squamous cell | ENST00000590986 | CTG/TTG | CAG/CAA | 1.4 |
| | cancer (length of survival) | | | | |
| Oncology | Esophageal squamous cell | ENST00000330794 | GTG/GTC | CAC/GAC | 1.2 |
| | carcinoma | | | | |
| Oncology | Esophageal squamous cell | ENST00000330794 | GTG/GTA | CAC/TAC | 5.2 |
| | carcinoma | | | | |
| Oncology | Esophageal squamous cell | ENST00000510817 | GTG/GTC | CAC/GAC | 1.2 |
| | carcinoma | | | | |
| Oncology | Esophageal squamous cell | ENST00000510817 | GTG/GTA | CAC/TAC | 5.2 |
| | carcinoma | | | | |
| Oncology | Glioblastoma | ENST00000320521 | CTC/CTT | GAG/AAG | 90.2 |
| Oncology | Lung cancer in ever | ENST00000326828 | TAC/TAT | GTA/ATA | 268.1 |
| | smokers | | | | |
| Oncology | Lung cancer in ever | ENST00000348639 | TAC/TAT | GTA/ATA | 268.1 |
| | smokers | | | | |
| Oncology | Lung cancer in ever | ENST00000559658 | TAC/TAT | GTA/ATA | 268.1 |
| | smokers | | | | |
| Oncology | Macrophage Migration | ENST00000344836 | GTG/GTC | CAC/GAC | 1.2 |
| | Inhibitory Factor levels | | | | |
| Oncology | Macrophage Migration | ENST00000381886 | GTG/GTC | CAC/GAC | 1.2 |
| | Inhibitory Factor levels | | | | |
| Oncology | Oral cavity and pharyngeal | ENST00000209665 | AGG/AGA | CCT/TCT | 1.1 |
| | cancer | | | | |
| Oncology | Oral cavity and pharyngeal | ENST00000437033 | AGG/AGA | CCT/TCT | 1.1 |
| | cancer | | | | |
| Oncology | Oral cavity and pharyngeal | ENST00000476959 | AGG/AGA | CCT/TCT | 1.1 |
| | cancer | | | | |
| Oncology | Oral cavity and pharyngeal | ENST00000482593 | AGG/AGA | CCT/TCT | 1.1 |
| | cancer | | | | |
| Oncology | Pancreatic cancer | ENST00000162330 | CTC/CTT | GAG/AAG | 90.2 |
| Oncology | Pancreatic cancer | ENST00000393420 | CTC/CTT | GAG/AAG | 90.2 |
| Oncology | Pancreatic cancer | ENST00000393422 | CTC/CTT | GAG/AAG | 90.2 |
| Oncology | Pancreatic cancer | ENST00000418647 | CTC/CTT | GAG/AAG | 90.2 |
| Oncology | Pancreatic cancer | ENST00000420641 | CTC/CTT | GAG/AAG | 90.2 |
| Oncology | Pancreatic cancer | ENST00000535626 | CTC/CTT | GAG/AAG | 90.2 |
| Oncology | Pancreatic cancer | ENST00000538440 | CTC/CTT | GAG/AAG | 90.2 |
| Oncology | Pancreatic cancer | ENST00000542031 | CTC/CTT | GAG/AAG | 90.2 |
| Ophthalmology | Myopia | ENST00000358540 | GTG/GTA | CAC/TAC | 5.2 |
| Ophthalmology | Myopia | ENST00000396166 | GTG/GTA | CAC/TAC | 5.2 |
| Ophthalmology | Myopia | ENST00000396168 | GTG/GTA | CAC/TAC | 5.2 |
| Ophthalmology | Myopia | ENST00000396170 | GTG/GTA | CAC/TAC | 5.2 |
| Ophthalmology | Myopia | ENST00000477854 | GTG/GTA | CAC/TAC | 5.2 |
| Ophthalmology | Myopia (pathological) | ENST00000336812 | ACG/ACT | CGT/AGT | 190.9 |
| Ophthalmology | Myopia (pathological) | ENST00000336812 | ACG/ACT | CGT/AGT | 190.9 |
| Respiratory | Asthma | ENST00000427077 | CTA/TTA | TAG/TAA | 2.0 |
| Respiratory | Asthma | ENST00000346243 | TCC/TCT | GGA/AGA | 5.7 |
| Respiratory | Asthma | ENST00000346872 | TCC/TCT | GGA/AGA | 5.7 |
| Respiratory | Asthma | ENST00000350532 | TCC/TCT | GGA/AGA | 5.7 |
| Respiratory | Asthma | ENST00000351680 | TCC/TCT | GGA/AGA | 5.7 |
| Respiratory | Asthma | ENST00000377944 | TCC/TCT | GGA/AGA | 5.7 |
| Respiratory | Asthma | ENST00000377945 | TCC/TCT | GGA/AGA | 5.7 |
| Respiratory | Asthma | ENST00000377952 | TCC/TCT | GGA/AGA | 5.7 |
| Respiratory | Asthma | ENST00000377958 | TCC/TCT | GGA/AGA | 5.7 |
| Respiratory | Asthma | ENST00000394189 | TCC/TCT | GGA/AGA | 5.7 |
| Respiratory | Asthma | ENST00000439016 | TCC/TCT | GGA/AGA | 5.7 |
| Respiratory | Asthma | ENST00000439167 | TCC/TCT | GGA/AGA | 5.7 |
| Respiratory | Asthma | ENST00000467757 | TCC/TCT | GGA/AGA | 5.7 |
| Respiratory | Asthma | ENST00000535189 | TCC/TCT | GGA/AGA | 5.7 |
| Respiratory | Asthma | ENST00000583368 | TCC/TCT | GGA/AGA | 5.7 |
| Respiratory | Asthma | ENST00000623724 | TCC/TCT | GGA/AGA | 5.7 |
| Respiratory | Post bronchodilator | ENST00000225275 | AAC/AAT | GTT/ATT | 655.6 |
| | FEV1/FVC ratio in COPD | | | | |
| Respiratory | Post bronchodilator | ENST00000240335 | CCC/CCA | GGG/TGG | 1.5 |
| | FEV1/FVC ratio in COPD | | | | |
| Respiratory | Post bronchodilator | ENST00000240335 | CCC/CCG | GGG/CGG | 3.4 |
| | FEV1/FVC ratio in COPD | | | | |
| Respiratory | Post bronchodilator | ENST00000240335 | CCC/CCT | GGG/AGG | 25.7 |
| | FEV1/FVC ratio in COPD | | | | |
| Respiratory | Post bronchodilator | ENST00000393853 | CCC/CCA | GGG/TGG | 1.5 |
| | FEV1/FVC ratio in COPD | | | | |
| Respiratory | Post bronchodilator | ENST00000393853 | CCC/CCG | GGG/CGG | 3.4 |
| | FEVl/FVC ratio in COPD | | | | |
| Respiratory | Post bronchodilator | ENST00000393853 | CCC/CCT | GGG/AGG | 25.7 |
| | FEVl/FVC ratio in COPD | | | | |
| Respiratory | Post bronchodilator | ENST00000642491 | CCC/CCA | GGG/TGG | 1.5 |
| | FEVl/FVC ratio in COPD | | | | |
| Respiratory | Post bronchodilator | ENST00000642491 | CCC/CCG | GGG/CGG | 3.4 |
| | FEVl/FVC ratio in COPD | | | | |
| Respiratory | Post bronchodilator | ENST00000642491 | CCC/CCT | GGG/AGG | 25.7 |
| | FEVl/FVC ratio in COPD | | | | |
| Respiratory | Post bronchodilator | ENST00000644296 | CCC/CCA | GGG/TGG | 1.5 |
| | FEVl/FVC ratio in COPD | | | | |
| Respiratory | Post bronchodilator | ENST00000644296 | CCC/CCG | GGG/CGG | 3.4 |
| | FEVl/FVC ratio in COPD | | | | |
| Respiratory | Post bronchodilator | ENST00000644296 | CCC/CCT | GGG/AGG | 25.7 |
| | FEVl/FVC ratio in COPD | | | | |

### TREM, TREM Core Fragment and TREM Fragment

A “tRNA-based effector molecule” or “TREM” refers to an RNA molecule comprising one or more of the properties described herein. A TREM can comprise a non-naturally occurring modification, e.g., as provided in Tables 4, 5, 6 or 7.

In an embodiment, a TREM includes a TREM comprising a sequence of Formula A; a TREM core fragment comprising a sequence of Formula B; or a TREM fragment comprising a portion of a TREM which TREM comprises a sequence of Formula A.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2]. In an embodiment, [VL Domain] is optional. In an embodiment, [L1] is optional.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1. In an embodiment, y=0. In an embodiment, y=1.

In an embodiment, a TREM fragment comprises a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises: one, two, three or all or any combination of the following: a TREM half (e.g., from a cleavage in the ACH Domain, e.g., in the anticodon sequence, e.g., a 5′ half or a 3′ half); a 5′ fragment (e.g., a fragment comprising the 5′ end, e.g., from a cleavage in a DH Domain or the ACH Domain); a 3′ fragment (e.g., a fragment comprising the 3′ end, e.g., from a cleavage in the TH Domain); or an internal fragment (e.g., from a cleavage in any one of the ACH Domain, DH Domain or TH Domain). Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM, a TREM core fragment or a TREM fragment can be charged with an amino acid (e.g., a cognate amino acid); charged with a non-cognate amino acid (e.g., a mischarged TREM (mTREM)); or not charged with an amino acid (e.g., an uncharged TREM (uTREM)). In an embodiment, a TREM, a TREM core fragment or a TREM fragment can be charged with an amino acid selected from alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, the TREM, TREM core fragment or TREM fragment is a cognate TREM. In an embodiment, the TREM, TREM core fragment or TREM fragment is a non-cognate TREM. In an embodiment, the TREM, TREM core fragment or TREM fragment recognizes a codon provided in Table 2 or Table 3.

TABLE 2
List of codons
AAA
AAC
AAG
AAU
ACA
ACC
ACG
ACU
AGA
AGC
AGG
AGU
AUA
AUC
AUG
AUU
CAA
CAC
CAG
CAU
CCA
CCC
CCG
CCU
CGA
CGC
CGG
CGU
CUA
CUC
CUG
CUU
GAA
GAC
GAG
GAU
GCA
GCC
GCG
GCU
GGA
GGC
GGG
GGU
GUA
GUC
GUG
GUU
UAA
UAC
UAG
UAU
UCA
UCC
UCG
UCU
UGA
UGC
UGG
UGU
UUA
UUC
UUG
UUU

TABLE 3
Amino acids and corresponding codons

| Amino Acid | mRNA codons |
|---|---|
| Alanine | GCU, GCC, GCA, GCG |
| Arginine | CGU, CGC, CGA, CGG, AGA, |
| | AGG |
| Asparagine | AAU, AAC |
| Aspartate | GAU, GAC |
| Cysteine | UGU, UGC |
| Glutamate | GAA, GAG |
| Glutamine | CAA, CAG |
| Glycine | GGU, GGC, GGA, GGG |
| Histidine | CAU, CAC |
| Isoleucine | AUU, AUC, AUA |
| Leucine | UUA, UUG, CUU, CUC, CUA, CUG |
| Lysine | AAA, AAG |
| Methionine | AUG |
| Phenylalanine | UUU, UUC |
| Proline | CCU, CCC, CCA, CCG |
| Serine | UCU, UCC, UCA, UCG, AGU, AGC |
| Stop | UAA, UAG, UGA |
| Threonine | ACU, ACC, ACA, ACG |
| Tryptophan | UGG |
| Tyrosine | UAU, UAC |
| Valine | GUU, GUC, GUA, GUG |

In an embodiment, a TREM comprises a ribonucleic acid (RNA) sequence encoded by a deoxyribonucleic acid (DNA) sequence disclosed in Table 4, e.g., any one of SEQ ID NOs: 1-451 disclosed in Table 4. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 4, e.g., any one of SEQ ID NOs: 1-451 disclosed in Table 4. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 4, e.g., any one of SEQ ID NOs: 1-451 disclosed in Table 4.

In an embodiment, a TREM, a TREM core fragment, or TREM fragment comprises at least 5, 10, 15, 20, 25, or 30 consecutive nucleotides of an RNA sequence encoded by a DNA sequence disclosed in Table 4, e.g., at least 5, 10, 15, 20, 25, or 30 consecutive nucleotides of an RNA sequence encoded by any one of SEQ ID NOs: 1-451 disclosed in Table 4. In an embodiment, a TREM, a TREM core fragment, or TREM fragment comprises at least 5, 10, 15, 20, 25, or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 4, e.g., any one of SEQ ID NOs: 1-451 disclosed in Table 4. In an embodiment, a TREM, a TREM core fragment, or TREM fragment comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 4, e.g., any one of SEQ ID NOs: 1-451 disclosed in Table 4.

In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 4, e.g., any one of SEQ ID NOs: 1-451 disclosed in Table 4. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 4, e.g., any one of SEQ ID NOs: 1-451 disclosed in Table 4. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 4, e.g., any one of SEQ ID NOs: 1-451 disclosed in Table 4.

In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 4, e.g., any one of SEQ ID NOs: 1-451 disclosed in Table 4. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 4, e.g., any one of SEQ ID NOs: 1-451 disclosed in Table 4. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 4, e.g., any one of SEQ ID NOs: 1-451 disclosed in Table 4.

In an embodiment, a TREM core fragment or a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

TABLE 4
List of tRNA sequences

| SEQ | | |
|---|---|---|
| ID | | |
| NO | tRNA name | tRNA sequence |
| 1 | Ala_AGC_chr6:28763741-28763812 (−) | GGGGGTATAGCTCAGTGGTAGAGCGCGTGCTTAGCATGCACGAGGTCC |
| | | TGGGTTCGATCCCCAGTACCTCCA |
| 2 | Ala_AGC_chr6:26687485-26687557 (+) | GGGGAATTAGCTCAAGTGGTAGAGCGCTTGCTTAGCACGCAAGAGGTA |
| | | GTGGGATCGATGCCCACATTCTCCA |
| 3 | Ala_AGC_chr6:26572092-26572164 (−) | GGGGAATTAGCTCAAATGGTAGAGCGCTCGCTTAGCATGCGAGAGGTA |
| | | GCGGGATCGATGCCCGCATTCTCCA |
| 4 | Ala_AGC_chr6:26682715-26682787 (+) | GGGGAATTAGCTCAAGTGGTAGAGCGCTTGCTTAGCATGCAAGAGGTA |
| | | GTGGGATCGATGCCCACATTCTCCA |
| 5 | Ala_AGC_chr6:26705606-26705678 (+) | GGGGAATTAGCTCAAGCGGTAGAGCGCTTGCTTAGCATGCAAGAGGTA |
| | | GTGGGATCGATGCCCACATTCTCCA |
| 6 | Ala_AGC_chr6:26673590-26673662 (+) | GGGGAATTAGCTCAAGTGGTAGAGCGCTTGCTTAGCATGCAAGAGGTA |
| | | GTGGGATCAATGCCCACATTCTCCA |
| 7 | Ala_AGC_chr14:89445442-89445514 (+) | GGGGAATTAGCTCAAGTGGTAGAGCGCTCGCTTAGCATGCGAGAGGTA |
| | | GTGGGATCGATGCCCGCATTCTCCA |
| 8 | Ala_AGC_chr6:58196623-58196695 (−) | GGGGAATTAGCCCAAGTGGTAGAGCGCTTGCTTAGCATGCAAGAGGTA |
| | | GTGGGATCGATGCCCACATTCTCCA |
| 9 | Ala_AGC_chr6:28806221-28806292 (−) | GGGGGTGTAGCTCAGTGGTAGAGCGCGTGCTTAGCATGCACGAGGCCC |
| | | CGGGTTCAATCCCCGGCACCTCCA |
| 10 | Ala_AGC_chr6:28574933-28575004 (+) | GGGGGTGTAGCTCAGTGGTAGAGCGCGTGCTTAGCATGTACGAGGTCC |
| | | CGGGTTCAATCCCCGGCACCTCCA |
| 11 | Ala_AGC_chr6:28626014-28626085 (−) | GGGGATGTAGCTCAGTGGTAGAGCGCATGCTTAGCATGCATGAGGTCC |
| | | CGGGTTCGATCCCCAGCATCTCCA |
| 12 | Ala_AGC_chr6:28678366-28678437 (+) | GGGGGTGTAGCTCAGTGGTAGAGCGCGTGCTTAGCATGCACGAGGCCC |
| | | TGGGTTCAATCCCCAGCACCTCCA |
| 13 | Ala_AGC_chr6:28779849-28779920 (−) | GGGGGTATAGCTCAGCGGTAGAGCGCGTGCTTAGCATGCACGAGGTCC |
| | | TGGGTTCAATCCCCAATACCTCCA |
| 14 | Ala_AGC_chr6:28687481-28687552 (+) | GGGGGTGTAGCTCAGTGGTAGAGCGCGTGCTTAGCATGCACGAGGCCC |
| | | CGGGTTCAATCCCTGGCACCTCCA |
| 15 | Ala_AGC_chr2:27274082-27274154 (+) | GGGGGATTAGCTCAAATGGTAGAGCGCTCGCTTAGCATGCGAGAGGTA |
| | | GCGGGATCGATGCCCGCATCCTCCA |
| 16 | Ala_AGC_chr6:26730737-26730809 (+) | GGGGAATTAGCTCAGGCGGTAGAGCGCTCGCTTAGCATGCGAGAGGTA |
| | | GCGGGATCGACGCCCGCATTCTCCA |
| 17 | Ala_CGC_chr6:26553731-26553802 (+) | GGGGATGTAGCTCAGTGGTAGAGCGCATGCTTCGCATGTATGAGGTCC |
| | | CGGGTTCGATCCCCGGCATCTCCA |
| 18 | Ala_CGC_chr6:28641613-28641684 (−) | GGGGATGTAGCTCAGTGGTAGAGCGCATGCTTCGCATGTATGAGGCCC |
| | | CGGGTTCGATCCCCGGCATCTCCA |
| 19 | Ala_CGC_chr2:157257281-157257352 (+) | GGGGATGTAGCTCAGTGGTAGAGCGCGCGCTTCGCATGTGTGAGGTCC |
| | | CGGGTTCAATCCCCGGCATCTCCA |
| 20 | Ala_CGC_chr6:28697092-28697163 (+) | GGGGGTGTAGCTCAGTGGTAGAGCGCGTGCTTCGCATGTACGAGGCCC |
| | | CGGGTTCGACCCCCGGCTCCTCCA |
| 21 | Ala_TGC_chr6:28757547-28757618 (−) | GGGGGTGTAGCTCAGTGGTAGAGCGCATGCTTTGCATGTATGAGGTCC |
| | | CGGGTTCGATCCCCGGCACCTCCA |
| 22 | Ala_TGC_chr6:28611222-28611293 (+) | GGGGATGTAGCTCAGTGGTAGAGCGCATGCTTTGCATGTATGAGGTCC |
| | | CGGGTTCGATCCCCGGCATCTCCA |
| 23 | Ala_TGC_chr5:180633868-180633939 (+) | GGGGATGTAGCTCAGTGGTAGAGCGCATGCTTTGCATGTATGAGGCCC |
| | | CGGGTTCGATCCCCGGCATCTCCA |
| 24 | Ala_TGC_chr12:125424512-125424583 (+) | GGGGATGTAGCTCAGTGGTAGAGCGCATGCTTTGCACGTATGAGGCCC |
| | | CGGGTTCAATCCCCGGCATCTCCA |
| 25 | Ala_TGC_chr6:28785012-28785083 (−) | GGGGGTGTAGCTCAGTGGTAGAGCGCATGCTTTGCATGTATGAGGCCT |
| | | CGGGTTCGATCCCCGACACCTCCA |
| 26 | Ala_TGC_chr6:28726141-28726212 (−) | GGGGGTGTAGCTCAGTGGTAGAGCACATGCTTTGCATGTGTGAGGCCC |
| | | CGGGTTCGATCCCCGGCACCTCCA |
| 27 | Ala_TGC_chr6:28770577-28770647 (−) | GGGGGTGTAGCTCAGTGGTAGAGCGCATGCTTTGCATGTATGAGGCCT |
| | | CGGTTCGATCCCCGACACCTCCA |
| 28 | Arg_ACG_chr6:26328368-26328440 (+) | GGGCCAGTGGCGCAATGGATAACGCGTCTGACTACGGATCAGAAGATT |
| | | CCAGGTTCGACTCCTGGCTGGCTCG |
| 29 | Arg_ACG_chr3:45730491-45730563 (−) | GGGCCAGTGGCGCAATGGATAACGCGTCTGACTACGGATCAGAAGATT |
| | | CTAGGTTCGACTCCTGGCTGGCTCG |
| 30 | Arg_CCG_chr6:28710729-28710801 (−) | GGCCGCGTGGCCTAATGGATAAGGCGTCTGATTCCGGATCAGAAGATT |
| | | GAGGGTTCGAGTCCCTTCGTGGTCG |
| 31 | Arg_CCG_chr17:66016013-66016085 (−) | GACCCAGTGGCCTAATGGATAAGGCATCAGCCTCCGGAGCTGGGGATT |
| | | GTGGGTTCGAGTCCCATCTGGGTCG |
| 32 | Arg_CCT_chr17:73030001-73030073 (+) | GCCCCAGTGGCCTAATGGATAAGGCACTGGCCTCCTAAGCCAGGGATT |
| | | GTGGGTTCGAGTCCCACCTGGGGTA |
| 33 | Arg_CCT_chr17:73030526-73030598 (−) | GCCCCAGTGGCCTAATGGATAAGGCACTGGCCTCCTAAGCCAGGGATT |
| | | GTGGGTTCGAGTCCCACCTGGGGTG |
| 34 | Arg_CCT_chr16:3202901-3202973 (+) | GCCCCGGTGGCCTAATGGATAAGGCATTGGCCTCCTAAGCCAGGGATT |
| | | GTGGGTTCGAGTCCCACCCGGGGTA |
| 35 | Arg_CCT_chr7:139025446-139025518 (+) | GCCCCAGTGGCCTAATGGATAAGGCATTGGCCTCCTAAGCCAGGGATT |
| | | GTGGGTTCGAGTCCCATCTGGGGTG |
| 36 | Arg_CCT_chr16:3243918-3243990 (+) | GCCCCAGTGGCCTGATGGATAAGGTACTGGCCTCCTAAGCCAGGGATT |
| | | GTGGGTTCGAGTTCCACCTGGGGTA |
| 37 | Arg_TCG_chr15:89878304-89878376 (+) | GGCCGCGTGGCCTAATGGATAAGGCGTCTGACTTCGGATCAGAAGATT |
| | | GCAGGTTCGAGTCCTGCCGCGGTCG |
| 38 | Arg_TCG_chr6:26323046-26323118 (+) | GACCACGTGGCCTAATGGATAAGGCGTCTGACTTCGGATCAGAAGATT |
| | | GAGGGTTCGAATCCCTCCGTGGTTA |
| 39 | Arg_TCG_chr17:73031208-73031280 (+) | GACCGCGTGGCCTAATGGATAAGGCGTCTGACTTCGGATCAGAAGATT |
| | | GAGGGTTCGAGTCCCTTCGTGGTCG |
| 40 | Arg_TCG_chr6:26299905-26299977 (+) | GACCACGTGGCCTAATGGATAAGGCGTCTGACTTCGGATCAGAAGATT |
| | | GAGGGTTCGAATCCCTTCGTGGTTA |
| 41 | Arg_TCG_chr6:28510891-28510963 (−) | GACCACGTGGCCTAATGGATAAGGCGTCTGACTTCGGATCAGAAGATT |
| | | GAGGGTTCGAATCCCTTCGTGGTTG |
| 42 | Arg_TCG_chr9:112960803-112960875 (+) | GGCCGTGTGGCCTAATGGATAAGGCGTCTGACTTCGGATCAAAAGATT |
| | | GCAGGTTTGAGTTCTGCCACGGTCG |
| 43 | Arg_TCT_chr1:94313129-94313213 (+) | GGCTCCGTGGCGCAATGGATAGCGCATTGGACTTCTAGAGGCTGAAGG |
| | | CATTCAAAGGTTCCGGGTTCGAGTCCCGGCGGAGTCG |
| 44 | Arg_TCT_chr17:8024243-8024330 (+) | GGCTCTGTGGCGCAATGGATAGCGCATTGGACTTCTAGTGACGAATAG |
| | | AGCAATTCAAAGGTTGTGGGTTCGAATCCCACCAGAGTCG |
| 45 | Arg_TCT_chr9:131102355-131102445 (−) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCG |
| 46 | Arg_TCT_chr11:59318767-59318852 (+) | GGCTCTGTGGCGCAATGGATAGCGCATTGGACTTCTAGATAGTTAGAG |
| | | AAATTCAAAGGTTGTGGGTTCGAGTCCCACCAGAGTCG |
| 47 | Arg_TCT_chr1:159111401-159111474 (−) | GTCTCTGTGGCGCAATGGACGAGCGCGCTGGACTTCTAATCCAGAGGT |
| | | TCCGGGTTCGAGTCCCGGCAGAGATG |
| 48 | Arg_TCT_chr6:27529963-27530049 (+) | GGCTCTGTGGCGCAATGGATAGCGCATTGGACTTCTAGCCTAAATCAA |
| | | GAGATTCAAAGGTTGCGGGTTCGAGTCCCTCCAGAGTCG |
| 49 | Asn_GTT_chr1:161510031-161510104 (+) | GTCTCTGTGGCGCAATCGGTTAGCGCGTTCGGCTGTTAACCGAAAGGT |
| | | TGGTGGTTCGATCCCACCCAGGGACG |
| 50 | Asn_GTT_chr1:143879832-143879905 (−) | GTCTCTGTGGCGCAATCGGCTAGCGCGTTTGGCTGTTAACTAAAAGGTT |
| | | GGCGGTTCGAACCCACCCAGAGGCG |
| 51 | Asn_GTT_chr1:144301611-144301684 (+) | GTCTCTGTGGTGCAATCGGTTAGCGCGTTCCGCTGTTAACCGAAAGCTT |
| | | GGTGGTTCGAGCCCACCCAGGGATG |
| 52 | Asn_GTT_chr1:149326272-149326345 (−) | GTCTCTGTGGCGCAATCGGCTAGCGCGTTTGGCTGTTAACTAAAAAGTT |
| | | GGTGGTTCGAACACACCCAGAGGCG |
| 53 | Asn_GTT_chr1:148248115-148248188 (+) | GTCTCTGTGGCGCAATCGGTTAGCGCGTTCGGCTGTTAACCGAAAGGT |
| | | TGGTGGTTCGAGCCCACCCAGGGACG |
| 54 | Asn_GTT_chr1:148598314-148598387 (−) | GTCTCTGTGGCGCAATCGGTTAGCGCATTCGGCTGTTAACCGAAAGGT |
| | | TGGTGGTTCGAGCCCACCCAGGGACG |
| 55 | Asn_GTT_chr1:17216172-17216245 (+) | GTCTCTGTGGCGCAATCGGTTAGCGCGTTCGGCTGTTAACCGAAAGAT |
| | | TGGTGGTTCGAGCCCACCCAGGGACG |
| 56 | Asn_GTT_chr1:16847080-16847153 (−) | GTCTCTGTGGCGCAATCGGTTAGCGCGTTCGGCTGTTAACTGAAAGGTT |
| | | GGTGGTTCGAGCCCACCCAGGGACG |
| 57 | Asn_GTT_chr1:149230570-149230643 (−) | GTCTCTGTGGCGCAATGGGTTAGCGCGTTCGGCTGTTAACCGAAAGGT |
| | | TGGTGGTTCGAGCCCATCCAGGGACG |
| 58 | Asn_GTT_chr1:148000805-148000878 (+) | GTCTCTGTGGCGTAGTCGGTTAGCGCGTTCGGCTGTTAACCGAAAAGTT |
| | | GGTGGTTCGAGCCCACCCAGGAACG |
| 59 | Asn_GTT_chr1:149711798-149711871 (−) | GTCTCTGTGGCGCAATCGGCTAGCGCGTTTGGCTGTTAACTAAAAGGTT |
| | | GGTGGTTCGAACCCACCCAGAGGCG |
| 60 | Asn_GTT_chr1:145979034-145979107 (−) | GTCTCTGTGGCGCAATCGGTTAGCGCGTTCGGCTGTTAACTGAAAGGTT |
| | | AGTGGTTCGAGCCCACCCGGGGACG |
| 61 | Asp_GTC_chr12:98897281-98897352 (+) | TCCTCGTTAGTATAGTGGTTAGTATCCCCGCCTGTCACGCGGGAGACCG |
| | | GGGTTCAATTCCCCGACGGGGAG |
| 62 | Asp_GTC_chr1:161410615-161410686 (−) | TCCTCGTTAGTATAGTGGTGAGTATCCCCGCCTGTCACGCGGGAGACC |
| | | GGGGTTCGATTCCCCGACGGGGAG |
| 63 | Asp_GTC_chr6:27551236-27551307 (−) | TCCTCGTTAGTATAGTGGTGAGTGTCCCCGTCTGTCACGCGGGAGACC |
| | | GGGGTTCGATTCCCCGACGGGGAG |
| 64 | Cys_GCA_chr7:149007281-149007352 (+) | GGGGGCATAGCTCAGTGGTAGAGCATTTGACTGCAGATCAAGAGGTCC |
| | | CTGGTTCAAATCCAGGTGCCCCCT |
| 65 | Cys_GCA_chr7:149074601-149074672 (−) | GGGGGTATAGCTCAGGGGTAGAGCATTTGACTGCAGATCAAGAGGTCC |
| | | CTGGTTCAAATCCAGGTGCCCCCC |
| 66 | Cys_GCA_chr7:149112229-149112300 (−) | GGGGGTATAGCTTAGCGGTAGAGCATTTGACTGCAGATCAAGAGGTCC |
| | | CCGGTTCAAATCCGGGTGCCCCCT |
| 67 | Cys_GCA_chr7:149344046-149344117 (−) | GGGGGTATAGCTTAGGGGTAGAGCATTTGACTGCAGATCAAAAGGTCC |
| | | CTGGTTCAAATCCAGGTGCCCCTT |
| 68 | Cys_GCA_chr7:149052766-149052837 (−) | GGGGGTATAGCTCAGGGGTAGAGCATTTGACTGCAGATCAAGAGGTCC |
| | | CCAGTTCAAATCTGGGTGCCCCCT |
| 69 | Cys_GCA_chr17:37017937-37018008 (−) | GGGGGTATAGCTCAGGGGTAGAGCATTTGACTGCAGATCAAGAAGTCC |
| | | CCGGTTCAAATCCGGGTGCCCCCT |
| 70 | Cys_GCA_chr7:149281816-149281887 (+) | GGGGGTATAGCTCAGGGGTAGAGCATTTGACTGCAGATCAAGAGGTCT |
| | | CTGGTTCAAATCCAGGTGCCCCCT |
| 71 | Cys_GCA_chr7:149243631-149243702 (+) | GGGGGTATAGCTCAGGGGTAGAGCACTTGACTGCAGATCAAGAAGTCC |
| | | TTGGTTCAAATCCAGGTGCCCCCT |
| 72 | Cys_GCA_chr7:149388272-149388343 (−) | GGGGATATAGCTCAGGGGTAGAGCATTTGACTGCAGATCAAGAGGTCC |
| | | CCGGTTCAAATCCGGGTGCCCCCC |
| 73 | Cys_GCA_chr7:149072850-149072921 (−) | GGGGGTATAGTTCAGGGGTAGAGCATTTGACTGCAGATCAAGAGGTCC |
| | | CTGGTTCAAATCCAGGTGCCCCCT |
| 74 | Cys_GCA_chr7:149310156-149310227 (−) | GGGGGTATAGCTCAGGGGTAGAGCATTTGACTGCAAATCAAGAGGTCC |
| | | CTGATTCAAATCCAGGTGCCCCCT |
| 75 | Cys_GCA_chr4:124430005-124430076 (−) | GGGGGTATAGCTCAGTGGTAGAGCATTTGACTGCAGATCAAGAGGTCC |
| | | CCGGTTCAAATCCGGGTGCCCCCT |
| 76 | Cys_GCA_chr7:149295046-149295117 (+) | GGGCGTATAGCTCAGGGGTAGAGCATTTGACTGCAGATCAAGAGGTCC |
| | | CCAGTTCAAATCTGGGTGCCCCCT |
| 77 | Cys_GCA_chr7:149361915-149361986 (+) | GGGGGTATAGCTCACAGGTAGAGCATTTGACTGCAGATCAAGAGGTCC |
| | | CCGGTTCAAATCTGGGTGCCCCCT |
| 78 | Cys_GCA_chr7:149253802-149253871 (+) | GGGCGTATAGCTCAGGGGTAGAGCATTTGACTGCAGATCAAGAGGTCC |
| | | CCAGTTCAAATCTGGGTGCCCA |
| 79 | Cys_GCA_chr7:149292305-149292376 (−) | GGGGGTATAGCTCACAGGTAGAGCATTTGACTGCAGATCAAGAGGTCC |
| | | CCGGTTCAAATCCGGTTACTCCCT |
| 80 | Cys_GCA_chr7:149286164-149286235 (−) | GGGGGTATAGCTCAGGGGTAGAGCACTTGACTGCAGATCAAGAGGTCC |
| | | CTGGTTCAAATCCAGGTGCCCCCT |
| 81 | Cys_GCA_chr17:37025545-37025616 (−) | GGGGGTATAGCTCAGTGGTAGAGCATTTGACTGCAGATCAAGAGGTCC |
| | | CTGGTTCAAATCCGGGTGCCCCCT |
| 82 | Cys_GCA_chr15:80036997-80037069 (+) | GGGGGTATAGCTCAGTGGGTAGAGCATTTGACTGCAGATCAAGAGGTC |
| | | CCCGGTTCAAATCCGGGTGCCCCCT |
| 83 | Cys_GCA_chr3:131947944-131948015 (−) | GGGGGTGTAGCTCAGTGGTAGAGCATTTGACTGCAGATCAAGAGGTCC |
| | | CTGGTTCAAATCCAGGTGCCCCCT |
| 84 | Cys_GCA_chr1:93981834-93981906 (−) | GGGGGTATAGCTCAGGTGGTAGAGCATTTGACTGCAGATCAAGAGGTC |
| | | CCCGGTTCAAATCCGGGTGCCCCCT |
| 85 | Cys_GCA_chr14:73429679-73429750 (+) | GGGGGTATAGCTCAGGGGTAGAGCATTTGACTGCAGATCAAGAGGTCC |
| | | CCGGTTCAAATCCGGGTGCCCCCT |
| 86 | Cys_GCA_chr3:131950642-131950713 (−) | GGGGGTATAGCTCAGGGGTAGAGCATTTGACTGCAGATCAAGAGGTCC |
| | | CTGGTTCAAATCCAGGTGCCCCCT |
| 87 | Gln_CTG_chr6:18836402-18836473 (+) | GGTTCCATGGTGTAATGGTTAGCACTCTGGACTCTGAATCCAGCGATCC |
| | | GAGTTCAAATCTCGGTGGAACCT |
| 88 | Gln_CTG_chr6:27515531-27515602 (−) | GGTTCCATGGTGTAATGGTTAGCACTCTGGACTCTGAATCCAGCGATCC |
| | | GAGTTCAAGTCTCGGTGGAACCT |
| 89 | Gln_CTG_chr1:145963304-145963375 (+) | GGTTCCATGGTGTAATGGTGAGCACTCTGGACTCTGAATCCAGCGATC |
| | | CGAGTTCGAGTCTCGGTGGAACCT |
| 90 | Gln_CTG_chr1:147737382-147737453 (−) | GGTTCCATGGTGTAATGGTAAGCACTCTGGACTCTGAATCCAGCGATC |
| | | CGAGTTCGAGTCTCGGTGGAACCT |
| 91 | Gln_CTG_chr6:27263212-27263283 (+) | GGTTCCATGGTGTAATGGTTAGCACTCTGGACTCTGAATCCGGTAATCC |
| | | GAGTTCAAATCTCGGTGGAACCT |
| 92 | Gln_CTG_chr6:27759135-27759206 (−) | GGCCCCATGGTGTAATGGTCAGCACTCTGGACTCTGAATCCAGCGATC |
| | | CGAGTTCAAATCTCGGTGGGACCC |
| 93 | Gln_CTG_chr1:147800937-147801008 (+) | GGTTCCATGGTGTAATGGTAAGCACTCTGGACTCTGAATCCAGCCATCT |
| | | GAGTTCGAGTCTCTGTGGAACCT |
| 94 | Gln_TTG_chr17:47269890-47269961 (+) | GGTCCCATGGTGTAATGGTTAGCACTCTGGACTTTGAATCCAGCGATCC |
| | | GAGTTCAAATCTCGGTGGGACCT |
| 95 | Gln_TTG_chr6:28557156-28557227 (+) | GGTCCCATGGTGTAATGGTTAGCACTCTGGACTTTGAATCCAGCAATCC |
| | | GAGTTCGAATCTCGGTGGGACCT |
| 96 | Gln_TTG_chr6:26311424-26311495 (−) | GGCCCCATGGTGTAATGGTTAGCACTCTGGACTTTGAATCCAGCGATC |
| | | CGAGTTCAAATCTCGGTGGGACCT |
| 97 | Gln_TTG_chr6:145503859-145503930 (+) | GGTCCCATGGTGTAATGGTTAGCACTCTGGGCTTTGAATCCAGCAATCC |
| | | GAGTTCGAATCTTGGTGGGACCT |
| 98 | Glu_CTC_chr1:145399233-145399304 (−) | TCCCTGGTGGTCTAGTGGTTAGGATTCGGCGCTCTCACCGCCGCGGCCC |
| | | GGGTTCGATTCCCGGTCAGGGAA |
| 99 | Glu_CTC_chr1:249168447-249168518 (+) | TCCCTGGTGGTCTAGTGGTTAGGATTCGGCGCTCTCACCGCCGCGGCCC |
| | | GGGTTCGATTCCCGGTCAGGAAA |
| 100 | Glu_TTC_chr2:131094701-131094772 (−) | TCCCATATGGTCTAGCGGTTAGGATTCCTGGTTTTCACCCAGGTGGCCC |
| | | GGGTTCGACTCCCGGTATGGGAA |
| 101 | Glu_TTC_chr13:45492062-45492133 (−) | TCCCACATGGTCTAGCGGTTAGGATTCCTGGTTTTCACCCAGGCGGCCC |
| | | GGGTTCGACTCCCGGTGTGGGAA |
| 102 | Glu_TTC_chr1:17199078-17199149 (+) | TCCCTGGTGGTCTAGTGGCTAGGATTCGGCGCTTTCACCGCCGCGGCCC |
| | | GGGTTCGATTCCCGGCCAGGGAA |
| 103 | Glu_TTC_chr1:16861774-16861845 (−) | TCCCTGGTGGTCTAGTGGCTAGGATTCGGCGCTTTCACCGCCGCGGCCC |
| | | GGGTTCGATTCCCGGTCAGGGAA |
| 104 | Gly_CCC_chr1:16872434-16872504 (−) | GCATTGGTGGTTCAGTGGTAGAATTCTCGCCTCCCACGCGGGAGACCC |
| | | GGGTTCAATTCCCGGCCAATGCA |
| 105 | Gly_CCC_chr2:70476123-70476193 (−) | GCGCCGCTGGTGTAGTGGTATCATGCAAGATTCCCATTCTTGCGACCCG |
| | | GGTTCGATTCCCGGGCGGCGCA |
| 106 | Gly_CCC_chr17:19764175-19764245 (+) | GCATTGGTGGTTCAATGGTAGAATTCTCGCCTCCCACGCAGGAGACCC |
| | | AGGTTCGATTCCTGGCCAATGCA |
| 107 | Gly_GCC_chr1:161413094-161413164 (+) | GCATGGGTGGTTCAGTGGTAGAATTCTCGCCTGCCACGCGGGAGGCCC |
| | | GGGTTCGATTCCCGGCCCATGCA |
| 108 | Gly_GCC_chr1:161493637-161493707 (−) | GCATTGGTGGTTCAGTGGTAGAATTCTCGCCTGCCACGCGGGAGGCCC |
| | | GGGTTCGATTCCCGGCCAATGCA |
| 109 | Gly_GCC_chr16:70812114-70812184 (−) | GCATTGGTGGTTCAGTGGTAGAATTCTCGCCTGCCACGCGGGAGGCCC |
| | | GGGTTTGATTCCCGGCCAGTGCA |
| 110 | Gly_GCC_chr1:161450356-161450426 (+) | GCATAGGTGGTTCAGTGGTAGAATTCTTGCCTGCCACGCAGGAGGCCC |
| | | AGGTTTGATTCCTGGCCCATGCA |
| 111 | Gly_GCC_chr16:70822597-70822667 (+) | GCATTGGTGGTTCAGTGGTAGAATTCTCGCCTGCCATGCGGGCGGCCG |
| | | GGCTTCGATTCCTGGCCAATGCA |
| 112 | Gly_TCC_chr19:4724082-4724153 (+) | GCGTTGGTGGTATAGTGGTTAGCATAGCTGCCTTCCAAGCAGTTGACC |
| | | CGGGTTCGATTCCCGGCCAACGCA |
| 113 | Gly_TCC_chr1:145397864-145397935 (−) | GCGTTGGTGGTATAGTGGTGAGCATAGCTGCCTTCCAAGCAGTTGACC |
| | | CGGGTTCGATTCCCGGCCAACGCA |
| 114 | Gly_TCC_chr17:8124866-8124937 (+) | GCGTTGGTGGTATAGTGGTAAGCATAGCTGCCTTCCAAGCAGTTGACC |
| | | CGGGTTCGATTCCCGGCCAACGCA |
| 115 | Gly_TCC_chr1:161409961-161410032 (−) | GCGTTGGTGGTATAGTGGTGAGCATAGTTGCCTTCCAAGCAGTTGACC |
| | | CGGGCTCGATTCCCGCCCAACGCA |
| 116 | His_GTG_chr1:145396881-145396952 (−) | GCCGTGATCGTATAGTGGTTAGTACTCTGCGTTGTGGCCGCAGCAACCT |
| | | CGGTTCGAATCCGAGTCACGGCA |
| 117 | His_GTG_chr1:149155828-149155899 (−) | GCCATGATCGTATAGTGGTTAGTACTCTGCGCTGTGGCCGCAGCAACC |
| | | TCGGTTCGAATCCGAGTCACGGCA |
| 118 | Ile_AAT_chr6:58149254-58149327 (+) | GGCCGGTTAGCTCAGTTGGTTAGAGCGTGGCGCTAATAACGCCAAGGT |
| | | CGCGGGTTCGATCCCCGTACGGGCCA |
| 119 | Ile_AAT_chr6:27655967-27656040 (+) | GGCCGGTTAGCTCAGTTGGTTAGAGCGTGGTGCTAATAACGCCAAGGT |
| | | CGCGGGTTCGATCCCCGTACTGGCCA |
| 120 | Ile_AAT_chr6:27242990-27243063 (−) | GGCTGGTTAGCTCAGTTGGTTAGAGCGTGGTGCTAATAACGCCAAGGT |
| | | CGCGGGTTCGATCCCCGTACTGGCCA |
| 121 | Ile_AAT_chr17:8130309-8130382 (−) | GGCCGGTTAGCTCAGTTGGTTAGAGCGTGGTGCTAATAACGCCAAGGT |
| | | CGCGGGTTCGAACCCCGTACGGGCCA |
| 122 | Ile_AAT_chr6:26554350-26554423 (+) | GGCCGGTTAGCTCAGTTGGTTAGAGCGTGGTGCTAATAACGCCAAGGT |
| | | CGCGGGTTCGATCCCCGTACGGGCCA |
| 123 | Ile_AAT_chr6:26745255-26745328 (−) | GGCCGGTTAGCTCAGTTGGTTAGAGCGTGGTGCTAATAACGCTAAGGT |
| | | CGCGGGTTCGATCCCCGTACTGGCCA |
| 124 | Ile_AAT_chr6:26721221-26721294 (−) | GGCCGGTTAGCTCAGTTGGTCAGAGCGTGGTGCTAATAACGCCAAGGT |
| | | CGCGGGTTCGATCCCCGTACGGGCCA |
| 125 | Ile_AAT_chr6:27636362-27636435 (+) | GGCCGGTTAGCTCAGTCGGCTAGAGCGTGGTGCTAATAACGCCAAGGT |
| | | CGCGGGTTCGATCCCCGTACGGGCCA |
| 126 | Ile_AAT_chr6:27241739-27241812 (+) | GGCTGGTTAGTTCAGTTGGTTAGAGCGTGGTGCTAATAACGCCAAGGT |
| | | CGTGGGTTCGATCCCCATATCGGCCA |
| 127 | Ile_GAT_chrX:3756418-3756491 (−) | GGCCGGTTAGCTCAGTTGGTAAGAGCGTGGTGCTGATAACACCAAGGT |
| | | CGCGGGCTCGACTCCCGCACCGGCCA |
| 128 | Ile_TAT_chr19:39902808-39902900 (−) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCA |
| 129 | Ile_TAT_chr2:43037676-43037768 (+) | GCTCCAGTGGCGCAATCGGTTAGCGCGCGGTACTTATACAGCAGTACA |
| | | TGCAGAGCAATGCCGAGGTTGTGAGTTCGAGCCTCACCTGGAGCA |
| 130 | Ile_TAT_chr6:26988125-26988218 (+) | GCTCCAGTGGCGCAATCGGTTAGCGCGCGGTACTTATATGGCAGTATG |
| | | TGTGCGAGTGATGCCGAGGTTGTGAGTTCGAGCCTCACCTGGAGCA |
| 131 | Ile_TAT_chr6:27599200-27599293 (+) | GCTCCAGTGGCGCAATCGGTTAGCGCGCGGTACTTATACAACAGTATA |
| | | TGTGCGGGTGATGCCGAGGTTGTGAGTTCGAGCCTCACCTGGAGCA |
| 132 | Ile_TAT_chr6:28505367-28505460 (+) | GCTCCAGTGGCGCAATCGGTTAGCGCGCGGTACTTATAAGACAGTGCA |
| | | CCTGTGAGCAATGCCGAGGTTGTGAGTTCAAGCCTCACCTGGAGCA |
| 133 | Leu_AAG_chr5:180524474-180524555 (−) | GGTAGCGTGGCCGAGCGGTCTAAGGCGCTGGATTAAGGCTCCAGTCTC |
| | | TTCGGAGGCGTGGGTTCGAATCCCACCGCTGCCA |
| 134 | Leu_AAG_chr5:180614701-180614782 (+) | GGTAGCGTGGCCGAGCGGTCTAAGGCGCTGGATTAAGGCTCCAGTCTC |
| | | TTCGGGGGCGTGGGTTCGAATCCCACCGCTGCCA |
| 135 | Leu_AAG_chr6:28956779-28956860 (+) | GGTAGCGTGGCCGAGCGGTCTAAGGCGCTGGATTAAGGCTCCAGTCTC |
| | | TTCGGGGGCGTGGGTTCAAATCCCACCGCTGCCA |
| 136 | Leu_AAG_chr6:28446400-28446481 (−) | GGTAGCGTGGCCGAGTGGTCTAAGACGCTGGATTAAGGCTCCAGTCTC |
| | | TTCGGGGGCGTGGGTTTGAATCCCACCGCTGCCA |
| 137 | Leu_CAA_chr6:28864000-28864105 (−) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| 138 | Leu_CAA_chr6:28908830-28908934 (+) | GTCAGGATGGCCGAGTGGTCTAAGGCGCCAGACTCAAGCTTGGCTTCC |
| | | TCGTGTTGAGGATTCTGGTCTCCAATGGAGGCGTGGGTTCGAATCCCA |
| 139 | Leu_CAA_chr6:27573417-27573524 (−) | GTCAGGATGGCCGAGTGGTCTAAGGCGCCAGACTCAAGCTTACTGCTT |
| | | CCTGTGTTCGGGTCTTCTGGTCTCCGTATGGAGGCGTGGGTTCGAATCC |
| 140 | Leu_CAA_chr6:27570348-27570454 (−) | GTCAGGATGGCCGAGTGGTCTAAGGCGCCAGACTCAAGTTGCTACTTC |
| | | CCAGGTTTGGGGCTTCTGGTCTCCGCATGGAGGCGTGGGTTCGAATCC |
| 141 | Leu_CAA_chr1:249168054-249168159 (+) | GTCAGGATGGCCGAGTGGTCTAAGGCGCCAGACTCAAGGTAAGCACCT |
| | | TGCCTGCGGGCTTTCTGGTCTCCGGATGGAGGCGTGGGTTCGAATCCC |
| 142 | Leu_CAA_chr11:9296790-9296863 (+) | GCCTCCTTAGTGCAGTAGGTAGCGCATCAGTCTCAAAATCTGAATGGT |
| | | CCTGAGTTCAAGCCTCAGAGGGGGCA |
| 143 | Leu_CAA_chr1:161581736-161581819 (−) | GTCAGGATGGCCGAGCAGTCTTAAGGCGCTGCGTTCAAATCGCACCCT |
| | | CCGCTGGAGGCGTGGGTTCGAATCCCACTTTTGACA |
| 144 | Leu_CAG_chr1:161411323-161411405 (+) | GTCAGGATGGCCGAGCGGTCTAAGGCGCTGCGTTCAGGTCGCAGTCTC |
| | | CCCTGGAGGCGTGGGTTCGAATCCCACTCCTGACA |
| 145 | Leu_CAG_chr16:57333863-57333945 (+) | GTCAGGATGGCCGAGCGGTCTAAGGCGCTGCGTTCAGGTCGCAGTCTC |
| | | CCCTGGAGGCGTGGGTTCGAATCCCACTTCTGACA |
| 146 | Leu_TAA_chr6:144537684-144537766 (+) | ACCAGGATGGCCGAGTGGTTAAGGCGTTGGACTTAAGATCCAATGGAC |
| | | ATATGTCCGCGTGGGTTCGAACCCCACTCCTGGTA |
| 147 | Leu_TAA_chr6:27688898-27688980 (−) | ACCGGGATGGCCGAGTGGTTAAGGCGTTGGACTTAAGATCCAATGGGC |
| | | TGGTGCCCGCGTGGGTTCGAACCCCACTCTCGGTA |
| 148 | Leu_TAA_chr11:59319228-59319310 (+) | ACCAGAATGGCCGAGTGGTTAAGGCGTTGGACTTAAGATCCAATGGAT |
| | | TCATATCCGCGTGGGTTCGAACCCCACTTCTGGTA |
| 149 | Leu_TAA_chr6:27198334-27198416 (−) | ACCGGGATGGCTGAGTGGTTAAGGCGTTGGACTTAAGATCCAATGGAC |
| | | AGGTGTCCGCGTGGGTTCGAGCCCCACTCCCGGTA |
| 150 | Leu_TAG_chr17:8023632-8023713 (−) | GGTAGCGTGGCCGAGCGGTCTAAGGCGCTGGATTTAGGCTCCAGTCTC |
| | | TTCGGAGGCGTGGGTTCGAATCCCACCGCTGCCA |
| 151 | Leu_TAG_chr14:21093529-21093610 (+) | GGTAGTGTGGCCGAGCGGTCTAAGGCGCTGGATTTAGGCTCCAGTCTC |
| | | TTCGGGGGCGTGGGTTCGAATCCCACCACTGCCA |
| 152 | Leu_TAG_chr16:22207032-22207113 (−) | GGTAGCGTGGCCGAGTGGTCTAAGGCGCTGGATTTAGGCTCCAGTCAT |
| | | TTCGATGGCGTGGGTTCGAATCCCACCGCTGCCA |
| 153 | Lys_CTT_chr14:58706613-58706685 (−) | GCCCGGCTAGCTCAGTCGGTAGAGCATGGGACTCTTAATCCCAGGGTC |
| | | GTGGGTTCGAGCCCCACGTTGGGCG |
| 154 | Lys_CTT_chr19:36066750-36066822 (+) | GCCCAGCTAGCTCAGTCGGTAGAGCATAAGACTCTTAATCTCAGGGTT |
| | | GTGGATTCGTGCCCCATGCTGGGTG |
| 155 | Lys_CTT_chr19:52425393-52425466 (−) | GCAGCTAGCTCAGTCGGTAGAGCATGAGACTCTTAATCTCAGGGTCAT |
| | | GGGTTCGTGCCCCATGTTGGGTGCCA |
| 156 | Lys_CTT_chr1:145395522-145395594 (−) | GCCCGGCTAGCTCAGTCGGTAGAGCATGAGACTCTTAATCTCAGGGTC |
| | | GTGGGTTCGAGCCCCACGTTGGGCG |
| 157 | Lys_CTT_chr16:3207406-3207478 (−) | GCCCGGCTAGCTCAGTCGGTAGAGCATGAGACCCTTAATCTCAGGGTC |
| | | GTGGGTTCGAGCCCCACGTTGGGCG |
| 158 | Lys_CTT_chr16:3241501-3241573 (+) | GCCCGGCTAGCTCAGTCGGTAGAGCATGGGACTCTTAATCTCAGGGTC |
| | | GTGGGTTCGAGCCCCACGTTGGGCG |
| 159 | Lys_CTT_chr16:3230555-3230627 (−) | GCCCGGCTAGCTCAGTCGATAGAGCATGAGACTCTTAATCTCAGGGTC |
| | | GTGGGTTCGAGCCGCACGTTGGGCG |
| 160 | Lys_CTT_chr1:55423542-55423614 (−) | GCCCAGCTAGCTCAGTCGGTAGAGCATGAGACTCTTAATCTCAGGGTC |
| | | ATGGGTTTGAGCCCCACGTTTGGTG |
| 161 | Lys_CTT_chr16:3214939-3215011 (+) | GCCTGGCTAGCTCAGTCGGCAAAGCATGAGACTCTTAATCTCAGGGTC |
| | | GTGGGCTCGAGCTCCATGTTGGGCG |
| 162 | Lys_CTT_chr5:26198539-26198611 (−) | GCCCGACTACCTCAGTCGGTGGAGCATGGGACTCTTCATCCCAGGGTT |
| | | GTGGGTTCGAGCCCCACATTGGGCA |
| 163 | Lys_TTT_chr16:73512216-73512288 (−) | GCCTGGATAGCTCAGTTGGTAGAGCATCAGACTTTTAATCTGAGGGTC |
| | | CAGGGTTCAAGTCCCTGTTCAGGCA |
| 164 | Lys_TTT_chr12:27843306-27843378 (+) | ACCCAGATAGCTCAGTCAGTAGAGCATCAGACTTTTAATCTGAGGGTC |
| | | CAAGGTTCATGTCCCTTTTTGGGTG |
| 165 | Lys_TTT_chr11:122430655-122430727 (+) | GCCTGGATAGCTCAGTTGGTAGAGCATCAGACTTTTAATCTGAGGGTC |
| | | CAGGGTTCAAGTCCCTGTTCAGGCG |
| 166 | Lys_TTT_chr1:204475655-204475727 (+) | GCCCGGATAGCTCAGTCGGTAGAGCATCAGACTTTTAATCTGAGGGTC |
| | | CAGGGTTCAAGTCCCTGTTCGGGCG |
| 167 | Lys_TTT_chr6:27559593-27559665 (−) | GCCTGGATAGCTCAGTCGGTAGAGCATCAGACTTTTAATCTGAGGGTC |
| | | CAGGGTTCAAGTCCCTGTTCAGGCG |
| 168 | Lys_TTT_chr11:59323902-59323974 (+) | GCCCGGATAGCTCAGTCGGTAGAGCATCAGACTTTTAATCTGAGGGTC |
| | | CGGGGTTCAAGTCCCTGTTCGGGCG |
| 169 | Lys_TTT_chr6:27302769-27302841 (−) | GCCTGGGTAGCTCAGTCGGTAGAGCATCAGACTTTTAATCTGAGGGTC |
| | | CAGGGTTCAAGTCCCTGTCCAGGCG |
| 170 | Lys_TTT_chr6:28715521-28715593 (+) | GCCTGGATAGCTCAGTTGGTAGAACATCAGACTTTTAATCTGACGGTG |
| | | CAGGGTTCAAGTCCCTGTTCAGGCG |
| 171 | Met_CAT_chr8:124169470-124169542 (−) | GCCTCGTTAGCGCAGTAGGTAGCGCGTCAGTCTCATAATCTGAAGGTC |
| | | GTGAGTTCGATCCTCACACGGGGCA |
| 172 | Met_CAT_chr16:71460396-71460468 (+) | GCCCTCTTAGCGCAGTGGGCAGCGCGTCAGTCTCATAATCTGAAGGTC |
| | | CTGAGTTCGAGCCTCAGAGAGGGCA |
| 173 | Met_CAT_chr6:28912352-28912424 (+) | GCCTCCTTAGCGCAGTAGGCAGCGCGTCAGTCTCATAATCTGAAGGTC |
| | | CTGAGTTCGAACCTCAGAGGGGGCA |
| 174 | Met_CAT_chr6:26735574-26735646 (−) | GCCCTCTTAGCGCAGCGGGCAGCGCGTCAGTCTCATAATCTGAAGGTC |
| | | CTGAGTTCGAGCCTCAGAGAGGGCA |
| 175 | Met_CAT_chr6:26701712-26701784 (+) | GCCCTCTTAGCGCAGCTGGCAGCGCGTCAGTCTCATAATCTGAAGGTC |
| | | CTGAGTTCAAGCCTCAGAGAGGGCA |
| 176 | Met_CAT_chr16:87417628-87417700 (−) | GCCTCGTTAGCGCAGTAGGCAGCGCGTCAGTCTCATAATCTGAAGGTC |
| | | GTGAGTTCGAGCCTCACACGGGGCA |
| 177 | Met_CAT_chr6:58168492-58168564 (−) | GCCCTCTTAGTGCAGCTGGCAGCGCGTCAGTTTCATAATCTGAAAGTCC |
| | | TGAGTTCAAGCCTCAGAGAGGGCA |
| 178 | Phe_GAA_chr6:28758499-28758571 (−) | GCCGAAATAGCTCAGTTGGGAGAGCGTTAGACTGAAGATCTAAAGGTC |
| | | CCTGGTTCGATCCCGGGTTTCGGCA |
| 179 | Phe_GAA_chr11:59333853-59333925 (−) | GCCGAAATAGCTCAGTTGGGAGAGCGTTAGACTGAAGATCTAAAGGTC |
| | | CCTGGTTCAATCCCGGGTTTCGGCA |
| 180 | Phe_GAA_chr6:28775610-28775682 (−) | GCCGAGATAGCTCAGTTGGGAGAGCGTTAGACTGAAGATCTAAAGGTC |
| | | CCTGGTTCAATCCCGGGTTTCGGCA |
| 181 | Phe_GAA_chr6:28791093-28791166 (−) | GCCGAAATAGCTCAGTTGGGAGAGCGTTAGACCGAAGATCTTAAAGGT |
| | | CCCTGGTTCAATCCCGGGTTTCGGCA |
| 182 | Phe_GAA_chr6:28731374-28731447 (−) | GCTGAAATAGCTCAGTTGGGAGAGCGTTAGACTGAAGATCTTAAAGTT |
| | | CCCTGGTTCAACCCTGGGTTTCAGCC |
| 183 | Pro_AGG_chr16:3241989-3242060 (+) | GGCTCGTTGGTCTAGGGGTATGATTCTCGCTTAGGATGCGAGAGGTCC |
| | | CGGGTTCAAATCCCGGACGAGCCC |
| 184 | Pro_AGG_chr1:167684725-167684796 (−) | GGCTCGTTGGTCTAGGGGTATGATTCTCGCTTAGGGTGCGAGAGGTCC |
| | | CGGGTTCAAATCCCGGACGAGCCC |
| 185 | Pro_CGG_chr1:167683962-167684033 (+) | GGCTCGTTGGTCTAGGGGTATGATTCTCGCTTCGGGTGCGAGAGGTCC |
| | | CGGGTTCAAATCCCGGACGAGCCC |
| 186 | Pro_CGG_chr6:27059521-27059592 (+) | GGCTCGTTGGTCTAGGGGTATGATTCTCGCTTCGGGTGTGAGAGGTCCC |
| | | GGGTTCAAATCCCGGACGAGCCC |
| 187 | Pro_TGG_chr14:21101165-21101236 (+) | GGCTCGTTGGTCTAGTGGTATGATTCTCGCTTTGGGTGCGAGAGGTCCC |
| | | GGGTTCAAATCCCGGACGAGCCC |
| 188 | Pro_TGG_chr11:75946869-75946940 (−) | GGCTCGTTGGTCTAGGGGTATGATTCTCGGTTTGGGTCCGAGAGGTCCC |
| | | GGGTTCAAATCCCGGACGAGCCC |
| 189 | Pro_TGG_chr5:180615854-180615925 (−) | GGCTCGTTGGTCTAGGGGTATGATTCTCGCTTTGGGTGCGAGAGGTCCC |
| | | GGGTTCAAATCCCGGACGAGCCC |
| 190 | SeC_TCA_chr19:45981859-45981945 (−) | GCCCGGATGATCCTCAGTGGTCTGGGGTGCAGGCTTCAAACCTGTAGC |
| | | TGTCTAGCGACAGAGTGGTTCAATTCCACCTTTCGGGCG |
| 191 | SeC_TCA_chr22:44546537-44546620 (+) | GCTCGGATGATCCTCAGTGGTCTGGGGTGCAGGCTTCAAACCTGTAGC |
| | | TGTCTAGTGACAGAGTGGTTCAATTCCACCTTTGTA |
| 192 | Ser_AGA_chr6:27509554-27509635 (−) | GTAGTCGTGGCCGAGTGGTTAAGGCGATGGACTAGAAATCCATTGGGG |
| | | TTTCCCCGCGCAGGTTCGAATCCTGCCGACTACG |
| 193 | Ser_AGA_chr6:26327817-26327898 (+) | GTAGTCGTGGCCGAGTGGTTAAGGCGATGGACTAGAAATCCATTGGGG |
| | | TCTCCCCGCGCAGGTTCGAATCCTGCCGACTACG |
| 194 | Ser_AGA_chr6:27499987-27500068 (+) | GTAGTCGTGGCCGAGTGGTTAAGGCGATGGACTAGAAATCCATTGGGG |
| | | TTTCCCCACGCAGGTTCGAATCCTGCCGACTACG |
| 195 | Ser_AGA_chr6:27521192-27521273 (−) | GTAGTCGTGGCCGAGTGGTTAAGGTGATGGACTAGAAACCCATTGGGG |
| | | TCTCCCCGCGCAGGTTCGAATCCTGCCGACTACG |
| 196 | Ser_CGA_chr17:8042199-8042280 (−) | GCTGTGATGGCCGAGTGGTTAAGGCGTTGGACTCGAAATCCAATGGGG |
| | | TCTCCCCGCGCAGGTTCGAATCCTGCTCACAGCG |
| 197 | Ser_CGA_chr6:27177628-27177709 (+) | GCTGTGATGGCCGAGTGGTTAAGGCGTTGGACTCGAAATCCAATGGGG |
| | | TCTCCCCGCGCAGGTTCAAATCCTGCTCACAGCG |
| 198 | Ser_CGA_chr6:27640229-27640310 (−) | GCTGTGATGGCCGAGTGGTTAAGGTGTTGGACTCGAAATCCAATGGGG |
| | | GTTCCCCGCGCAGGTTCAAATCCTGCTCACAGCG |
| 199 | Ser_CGA_chr12:56584148-56584229 (+) | GTCACGGTGGCCGAGTGGTTAAGGCGTTGGACTCGAAATCCAATGGGG |
| | | TTTCCCCGCACAGGTTCGAATCCTGTTCGTGACG |
| 200 | Ser_GCT_chr6:27065085-27065166 (+) | GACGAGGTGGCCGAGTGGTTAAGGCGATGGACTGCTAATCCATTGTGC |
| | | TCTGCACGCGTGGGTTCGAATCCCACCCTCGTCG |
| 201 | Ser_GCT_chr6:27265775-27265856 (+) | GACGAGGTGGCCGAGTGGTTAAGGCGATGGACTGCTAATCCATTGTGC |
| | | TCTGCACGCGTGGGTTCGAATCCCACCTTCGTCG |
| 202 | Ser_GCT_chr11:66115591-66115672 (+) | GACGAGGTGGCCGAGTGGTTAAGGCGATGGACTGCTAATCCATTGTGC |
| | | TTTGCACGCGTGGGTTCGAATCCCATCCTCGTCG |
| 203 | Ser_GCT_chr6:28565117-28565198 (−) | GACGAGGTGGCCGAGTGGTTAAGGCGATGGACTGCTAATCCATTGTGC |
| | | TCTGCACGCGTGGGTTCGAATCCCATCCTCGTCG |
| 204 | Ser_GCT_chr6:28180815-28180896 (+) | GACGAGGTGGCCGAGTGGTTAAGGCGATGGACTGCTAATCCATTGTGC |
| | | TCTGCACACGTGGGTTCGAATCCCATCCTCGTCG |
| 205 | Ser_GCT_chr6:26305718-26305801 (−) | GGAGAGGCCTGGCCGAGTGGTTAAGGCGATGGACTGCTAATCCATTGT |
| | | GCTCTGCACGCGTGGGTTCGAATCCCATCCTCGTCG |
| 206 | Ser_TGA_chr10:69524261-69524342 (+) | GCAGCGATGGCCGAGTGGTTAAGGCGTTGGACTTGAAATCCAATGGGG |
| | | TCTCCCCGCGCAGGTTCGAACCCTGCTCGCTGCG |
| 207 | Ser_TGA_chr6:27513468-27513549 (+) | GTAGTCGTGGCCGAGTGGTTAAGGCGATGGACTTGAAATCCATTGGGG |
| | | TTTCCCCGCGCAGGTTCGAATCCTGCCGACTACG |
| 208 | Ser_TGA_chr6:26312824-26312905 (−) | GTAGTCGTGGCCGAGTGGTTAAGGCGATGGACTTGAAATCCATTGGGG |
| | | TCTCCCCGCGCAGGTTCGAATCCTGCCGACTACG |
| 209 | Ser_TGA_chr6:27473607-27473688 (−) | GTAGTCGTGGCCGAGTGGTTAAGGCGATGGACTTGAAATCCATTGGGG |
| | | TTTCCCCGCGCAGGTTCGAATCCTGTCGGCTACG |
| 210 | Thr_AGT_chr17:8090478-8090551 (+) | GGCGCCGTGGCTTAGTTGGTTAAAGCGCCTGTCTAGTAAACAGGAGAT |
| | | CCTGGGTTCGAATCCCAGCGGTGCCT |
| 211 | Thr_AGT_chr6:26533145-26533218 (−) | GGCTCCGTGGCTTAGCTGGTTAAAGCGCCTGTCTAGTAAACAGGAGAT |
| | | CCTGGGTTCGAATCCCAGCGGGGCCT |
| 212 | Thr_AGT_chr6:28693795-28693868 (+) | GGCTCCGTAGCTTAGTTGGTTAAAGCGCCTGTCTAGTAAACAGGAGAT |
| | | CCTGGGTTCGACTCCCAGCGGGGCCT |
| 213 | Thr_AGT_chr6:27694473-27694546 (+) | GGCTTCGTGGCTTAGCTGGTTAAAGCGCCTGTCTAGTAAACAGGAGAT |
| | | CCTGGGTTCGAATCCCAGCGAGGCCT |
| 214 | Thr_AGT_chr17:8042770-8042843 (−) | GGCGCCGTGGCTTAGCTGGTTAAAGCGCCTGTCTAGTAAACAGGAGAT |
| | | CCTGGGTTCGAATCCCAGCGGTGCCT |
| 215 | Thr_AGT_chr6:27130050-27130123 (+) | GGCCCTGTGGCTTAGCTGGTCAAAGCGCCTGTCTAGTAAACAGGAGAT |
| | | CCTGGGTTCGAATCCCAGCGGGGCCT |
| 216 | Thr_CGT_chr6:28456770-28456843 (−) | GGCTCTATGGCTTAGTTGGTTAAAGCGCCTGTCTCGTAAACAGGAGAT |
| | | CCTGGGTTCGACTCCCAGTGGGGCCT |
| 217 | Thr_CGT_chr16:14379750-14379821 (+) | GGCGCGGTGGCCAAGTGGTAAGGCGTCGGTCTCGTAAACCGAAGATCA |
| | | CGGGTTCGAACCCCGTCCGTGCCT |
| 218 | Thr_CGT_chr6:28615984-28616057 (−) | GGCTCTGTGGCTTAGTTGGCTAAAGCGCCTGTCTCGTAAACAGGAGAT |
| | | CCTGGGTTCGAATCCCAGCGGGGCCT |
| 219 | Thr_CGT_chr17:29877093-29877164 (+) | GGCGCGGTGGCCAAGTGGTAAGGCGTCGGTCTCGTAAACCGAAGATCG |
| | | CGGGTTCGAACCCCGTCCGTGCCT |
| 220 | Thr_CGT_chr6:27586135-27586208 (+) | GGCCCTGTAGCTCAGCGGTTGGAGCGCTGGTCTCGTAAACCTAGGGGT |
| | | CGTGAGTTCAAATCTCACCAGGGCCT |
| 221 | Thr_TGT_chr6:28442329-28442402 (−) | GGCTCTATGGCTTAGTTGGTTAAAGCGCCTGTCTTGTAAACAGGAGAT |
| | | CCTGGGTTCGAATCCCAGTAGAGCCT |
| 222 | Thr_TGT_chr1:222638347-222638419 (+) | GGCTCCATAGCTCAGTGGTTAGAGCACTGGTCTTGTAAACCAGGGGTC |
| | | GCGAGTTCGATCCTCGCTGGGGCCT |
| 223 | Thr_TGT_chr14:21081949-21082021 (−) | GGCTCCATAGCTCAGGGGTTAGAGCGCTGGTCTTGTAAACCAGGGGTC |
| | | GCGAGTTCAATTCTCGCTGGGGCCT |
| 224 | Thr_TGT_chr14:21099319-21099391 (−) | GGCTCCATAGCTCAGGGGTTAGAGCACTGGTCTTGTAAACCAGGGGTC |
| | | GCGAGTTCAAATCTCGCTGGGGCCT |
| 225 | Thr_TGT_chr14:21149849-21149921 (+) | GGCCCTATAGCTCAGGGGTTAGAGCACTGGTCTTGTAAACCAGGGGTC |
| | | GCGAGTTCAAATCTCGCTGGGGCCT |
| 226 | Thr_TGT_chr5:180618687-180618758 (−) | GGCTCCATAGCTCAGGGGTTAGAGCACTGGTCTTGTAAACCAGGGTCG |
| | | CGAGTTCAAATCTCGCTGGGGCCT |
| 227 | Trp_CCA_chr17:8124187-8124258 (−) | GGCCTCGTGGCGCAACGGTAGCGCGTCTGACTCCAGATCAGAAGGTTG |
| | | CGTGTTCAAATCACGTCGGGGTCA |
| 228 | Trp_CCA_chr17:19411494-19411565 (+) | GACCTCGTGGCGCAATGGTAGCGCGTCTGACTCCAGATCAGAAGGTTG |
| | | CGTGTTCAAGTCACGTCGGGGTCA |
| 229 | Trp_CCA_chr6:26319330-26319401 (−) | GACCTCGTGGCGCAACGGTAGCGCGTCTGACTCCAGATCAGAAGGTTG |
| | | CGTGTTCAAATCACGTCGGGGTCA |
| 230 | Trp_CCA_chr12:98898030-98898101 (+) | GACCTCGTGGCGCAACGGTAGCGCGTCTGACTCCAGATCAGAAGGCTG |
| | | CGTGTTCGAATCACGTCGGGGTCA |
| 231 | Trp_CCA_chr7:99067307-99067378 (+) | GACCTCGTGGCGCAACGGCAGCGCGTCTGACTCCAGATCAGAAGGTTG |
| | | CGTGTTCAAATCACGTCGGGGTCA |
| 232 | Tyr_ATA_chr2:219110549-219110641 (+) | CCTTCAATAGTTCAGCTGGTAGAGCAGAGGACTATAGCTACTTCCTCA |
| | | GTAGGAGACGTCCTTAGGTTGCTGGTTCGATTCCAGCTTGAAGGA |
| 233 | Tyr_GTA_chr6:26569086-26569176 (+) | CCTTCGATAGCTCAGTTGGTAGAGCGGAGGACTGTAGTTGGCTGTGTC |
| | | CTTAGACATCCTTAGGTCGCTGGTTCGAATCCGGCTCGAAGGA |
| 234 | Tyr_GTA_chr2:27273650-27273738 (+) | CCTTCGATAGCTCAGTTGGTAGAGCGGAGGACTGTAGTGGATAGGGCG |
| | | TGGCAATCCTTAGGTCGCTGGTTCGATTCCGGCTCGAAGGA |
| 235 | Tyr_GTA_chr6:26577332-26577420 (+) | CCTTCGATAGCTCAGTTGGTAGAGCGGAGGACTGTAGGCTCATTAAGC |
| | | AAGGTATCCTTAGGTCGCTGGTTCGAATCCGGCTCGGAGGA |
| 236 | Tyr_GTA_chr14:21125623-21125716 (−) | CCTTCGATAGCTCAGCTGGTAGAGCGGAGGACTGTAGATTGTATAGAC |
| | | ATTTGCGGACATCCTTAGGTCGCTGGTTCGATTCCAGCTCGAAGGA |
| 237 | Tyr_GTA_chr8:67025602-67025694 (+) | CCTTCGATAGCTCAGCTGGTAGAGCGGAGGACTGTAGCTACTTCCTCA |
| | | GCAGGAGACATCCTTAGGTCGCTGGTTCGATTCCGGCTCGAAGGA |
| 238 | Tyr_GTA_chr8:67026223-67026311 (+) | CCTTCGATAGCTCAGCTGGTAGAGCGGAGGACTGTAGGCGCGCGCCCG |
| | | TGGCCATCCTTAGGTCGCTGGTTCGATTCCGGCTCGAAGGA |
| 239 | Tyr_GTA_chr14:21121258-21121351 (−) | CCTTCGATAGCTCAGCTGGTAGAGCGGAGGACTGTAGCCTGTAGAAAC |
| | | ATTTGTGGACATCCTTAGGTCGCTGGTTCGATTCCGGCTCGAAGGA |
| 240 | Tyr_GTA_chr14:21131351-21131444 (−) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGA |
| 241 | Tyr_GTA_chr14:21151432-21151520 (+) | CCTTCGATAGCTCAGCTGGTAGAGCGGAGGACTGTAGTACTTAATGTG |
| | | TGGTCATCCTTAGGTCGCTGGTTCGATTCCGGCTCGAAGGA |
| 242 | Tyr_GTA_chr6:26595102-26595190 (+) | CCTTCGATAGCTCAGCTGGTAGAGCGGAGGACTGTAGGGGTTTGAATG |
| | | TGGTCATCCTTAGGTCGCTGGTTCGAATCCGGCTCGGAGGA |
| 243 | Tyr_GTA_chr14:21128117-21128210 (−) | CCTTCGATAGCTCAGCTGGTAGAGCGGAGGACTGTAGACTGCGGAAAC |
| | | GTTTGTGGACATCCTTAGGTCGCTGGTTCAATTCCGGCTCGAAGGA |
| 244 | Tyr_GTA_chr6:26575798-26575887 (+) | CTTTCGATAGCTCAGTTGGTAGAGCGGAGGACTGTAGGTTCATTAAAC |
| | | TAAGGCATCCTTAGGTCGCTGGTTCGAATCCGGCTCGAAGGA |
| 245 | Tyr_GTA_chr8:66609532-66609619 (−) | TCTTCAATAGCTCAGCTGGTAGAGCGGAGGACTGTAGGTGCACGCCCG |
| | | TGGCCATTCTTAGGTGCTGGTTTGATTCCGACTTGGAGAG |
| 246 | Val_AAC_chr3:169490018-169490090 (+) | GTTTCCGTAGTGTAGTGGTTATCACGTTCGCCTAACACGCGAAAGGTCC |
| | | CCGGTTCGAAACCGGGCGGAAACA |
| 247 | Val_AAC_chr5:180615416-180615488 (−) | GTTTCCGTAGTGTAGTGGTCATCACGTTCGCCTAACACGCGAAAGGTC |
| | | CCCGGTTCGAAACCGGGCGGAAACA |
| 248 | Val_AAC_chr6:27618707-27618779 (−) | GTTTCCGTAGTGTAGTGGTTATCACGTTCGCCTAACACGCGAAAGGTCC |
| | | CTGGATCAAAACCAGGCGGAAACA |
| 249 | Val_AAC_chr6:27648885-27648957 (−) | GTTTCCGTAGTGTAGTGGTTATCACGTTCGCCTAACACGCGAAAGGTCC |
| | | GCGGTTCGAAACCGGGCGGAAACA |
| 250 | Val_AAC_chr6:27203288-27203360 (+) | GTTTCCGTAGTGTAGTGGTTATCACGTTTGCCTAACACGCGAAAGGTCC |
| | | CCGGTTCGAAACCGGGCAGAAACA |
| 251 | Val_AAC_chr6:28703206-28703277 (−) | GGGGGTGTAGCTCAGTGGTAGAGCGTATGCTTAACATTCATGAGGCTC |
| | | TGGGTTCGATCCCCAGCACTTCCA |
| 252 | Val_CAC_chr1:161369490-161369562 (−) | GTTTCCGTAGTGTAGTGGTTATCACGTTCGCCTCACACGCGAAAGGTCC |
| | | CCGGTTCGAAACCGGGCGGAAACA |
| 253 | Val_CAC_chr6:27248049-27248121 (−) | GCTTCTGTAGTGTAGTGGTTATCACGTTCGCCTCACACGCGAAAGGTCC |
| | | CCGGTTCGAAACCGGGCAGAAGCA |
| 254 | Val_CAC_chr19:4724647-4724719 (−) | GTTTCCGTAGTGTAGCGGTTATCACATTCGCCTCACACGCGAAAGGTCC |
| | | CCGGTTCGATCCCGGGCGGAAACA |
| 255 | Val_CAC_chr1:149298555-149298627 (−) | GTTTCCGTAGTGTAGTGGTTATCACGTTCGCCTCACACGCGAAAGGTCC |
| | | CCGGTTCGAAACTGGGCGGAAACA |
| 256 | Val_CAC_chr1:149684088-149684161 (−) | GTTTCCGTAGTGTAGTGGTTATCACGTTCGCCTCACACGCGTAAAGGTC |
| | | CCCGGTTCGAAACCGGGCGGAAACA |
| 257 | Val_CAC_chr6:27173867-27173939 (−) | GTTTCCGTAGTGGAGTGGTTATCACGTTCGCCTCACACGCGAAAGGTC |
| | | CCCGGTTTGAAACCAGGCGGAAACA |
| 258 | Val_TAC_chr11:59318102-59318174 (−) | GGTTCCATAGTGTAGTGGTTATCACGTCTGCTTTACACGCAGAAGGTCC |
| | | TGGGTTCGAGCCCCAGTGGAACCA |
| 259 | Val_TAC_chr11:59318460-59318532 (−) | GGTTCCATAGTGTAGCGGTTATCACGTCTGCTTTACACGCAGAAGGTCC |
| | | TGGGTTCGAGCCCCAGTGGAACCA |
| 260 | Val_TAC_chr10:5895674-5895746 (−) | GGTTCCATAGTGTAGTGGTTATCACATCTGCTTTACACGCAGAAGGTCC |
| | | TGGGTTCAAGCCCCAGTGGAACCA |
| 261 | Val_TAC_chr6:27258405-27258477 (+) | GTTTCCGTGGTGTAGTGGTTATCACATTCGCCTTACACGCGAAAGGTCC |
| | | TCGGGTCGAAACCGAGCGGAAACA |
| 262 | iMet_CAT_chr1:153643726-153643797 (+) | AGCAGAGTGGCGCAGCGGAAGCGTGCTGGGCCCATAACCCAGAGGTC |
| | | GATGGATCGAAACCATCCTCTGCTA |
| 263 | iMet_CAT_chr6:27745664-27745735 (+) | AGCAGAGTGGCGCAGCGGAAGCGTGCTGGGCCCATAACCCAGAGGTC |
| | | GATGGATCTAAACCATCCTCTGCTA |
| 264 | Glu_TTC_chr1:16861773-16861845 (−) | TCCCTGGTGGTCTAGTGGCTAGGATTCGGCGCTTTCACCGCCGCGGCCC |
| | | GGGTTCGATTCCCGGTCAGGGAAT |
| 265 | Gly_CCC_chr1:17004765-17004836 (−) | GCGTTGGTGGTTTAGTGGTAGAATTCTCGCCTCCCATGCGGGAGACCC |
| | | GGGTTCAATTCCCGGCCACTGCAC |
| 266 | Gly_CCC_chr1:17053779-17053850 (+) | GGCCTTGGTGGTGCAGTGGTAGAATTCTCGCCTCCCACGTGGGAGACC |
| | | CGGGTTCAATTCCCGGCCAATGCA |
| 267 | Glu_TTC_chr1:17199077-17199149 (+) | GTCCCTGGTGGTCTAGTGGCTAGGATTCGGCGCTTTCACCGCCGCGGCC |
| | | CGGGTTCGATTCCCGGCCAGGGAA |
| 268 | Asn_GTT_chr1:17216171-17216245 (+) | TGTCTCTGTGGCGCAATCGGTTAGCGCGTTCGGCTGTTAACCGAAAGA |
| | | TTGGTGGTTCGAGCCCACCCAGGGACG |
| 269 | Arg_TCT_chr1:94313128-94313213 (+) | TGGCTCCGTGGCGCAATGGATAGCGCATTGGACTTCTAGAGGCTGAAG |
| | | GCATTCAAAGGTTCCGGGTTCGAGTCCCGGCGGAGTCG |
| 270 | Lys_CTT_chr1:145395521-145395594 (−) | GCCCGGCTAGCTCAGTCGGTAGAGCATGAGACTCTTAATCTCAGGGTC |
| | | GTGGGTTCGAGCCCCACGTTGGGCGC |
| 271 | His_GTG_chr1:145396880-145396952 (−) | GCCGTGATCGTATAGTGGTTAGTACTCTGCGTTGTGGCCGCAGCAACCT |
| | | CGGTTCGAATCCGAGTCACGGCAG |
| 272 | Gly_TCC_chr1:145397863-145397935 (−) | GCGTTGGTGGTATAGTGGTGAGCATAGCTGCCTTCCAAGCAGTTGACC |
| | | CGGGTTCGATTCCCGGCCAACGCAG |
| 273 | Glu_CTC_chr1:145399232-145399304 (−) | TCCCTGGTGGTCTAGTGGTTAGGATTCGGCGCTCTCACCGCCGCGGCCC |
| | | GGGTTCGATTCCCGGTCAGGGAAA |
| 274 | Gln_CTG_chr1:145963303-145963375 (+) | AGGTTCCATGGTGTAATGGTGAGCACTCTGGACTCTGAATCCAGCGAT |
| | | CCGAGTTCGAGTCTCGGTGGAACCT |
| 275 | Asn_GTT_chr1:148000804-148000878 (+) | TGTCTCTGTGGCGTAGTCGGTTAGCGCGTTCGGCTGTTAACCGAAAAGT |
| | | TGGTGGTTCGAGCCCACCCAGGAACG |
| 276 | Asn_GTT_chr1:148248114-148248188 (+) | TGTCTCTGTGGCGCAATCGGTTAGCGCGTTCGGCTGTTAACCGAAAGG |
| | | TTGGTGGTTCGAGCCCACCCAGGGACG |
| 277 | Asn_GTT_chr1:148598313-148598387 (−) | GTCTCTGTGGCGCAATCGGTTAGCGCATTCGGCTGTTAACCGAAAGGT |
| | | TGGTGGTTCGAGCCCACCCAGGGACGC |
| 278 | Asn_GTT_chr1:149230569-149230643 (−) | GTCTCTGTGGCGCAATGGGTTAGCGCGTTCGGCTGTTAACCGAAAGGT |
| | | TGGTGGTTCGAGCCCATCCAGGGACGC |
| 279 | Val_CAC_chr1:149294665-149294736 (−) | GCACTGGTGGTTCAGTGGTAGAATTCTCGCCTCACACGCGGGACACCC |
| | | GGGTTCAATTCCCGGTCAAGGCAA |
| 280 | Val_CAC_chr1:149298554-149298627 (−) | GTTTCCGTAGTGTAGTGGTTATCACGTTCGCCTCACACGCGAAAGGTCC |
| | | CCGGTTCGAAACTGGGCGGAAACAG |
| 281 | Gly_CCC_chr1:149680209-149680280 (−) | GCACTGGTGGTTCAGTGGTAGAATTCTCGCCTCCCACGCGGGAGACCC |
| | | GGGTTTAATTCCCGGTCAAGATAA |
| 282 | Val_CAC_chr1:149684087-149684161 (−) | GTTTCCGTAGTGTAGTGGTTATCACGTTCGCCTCACACGCGTAAAGGTC |
| | | CCCGGTTCGAAACCGGGCGGAAACAT |
| 283 | Met_CAT_chr1:153643725-153643797 (+) | TAGCAGAGTGGCGCAGCGGAAGCGTGCTGGGCCCATAACCCAGAGGT |
| | | CGATGGATCGAAACCATCCTCTGCTA |
| 284 | Val_CAC_chr1:161369489-161369562 (−) | GTTTCCGTAGTGTAGTGGTTATCACGTTCGCCTCACACGCGAAAGGTCC |
| | | CCGGTTCGAAACCGGGCGGAAACAA |
| 285 | Asp_GTC_chr1:161410614-161410686 (−) | TCCTCGTTAGTATAGTGGTGAGTATCCCCGCCTGTCACGCGGGAGACC |
| | | GGGGTTCGATTCCCCGACGGGGAGG |
| 286 | Gly_GCC_chr1:161413093-161413164 (+) | TGCATGGGTGGTTCAGTGGTAGAATTCTCGCCTGCCACGCGGGAGGCC |
| | | CGGGTTCGATTCCCGGCCCATGCA |
| 287 | Glu_CTC_chr1:161417017-161417089 (−) | TCCCTGGTGGTCTAGTGGTTAGGATTCGGCGCTCTCACCGCCGCGGCCC |
| | | GGGTTCGATTCCCGGTCAGGGAAG |
| 288 | Asp_GTC_chr1:161492934-161493006 (+) | ATCCTTGTTACTATAGTGGTGAGTATCTCTGCCTGTCATGCGTGAGAGA |
| | | GGGGGTCGATTCCCCGACGGGGAG |
| 289 | Gly_GCC_chr1:161493636-161493707 (−) | GCATTGGTGGTTCAGTGGTAGAATTCTCGCCTGCCACGCGGGAGGCCC |
| | | GGGTTCGATTCCCGGCCAATGCAC |
| 290 | Leu_CAG_chr1:161500131-161500214 (−) | GTCAGGATGGCCGAGCGGTCTAAGGCGCTGCGTTCAGGTCGCAGTCTC |
| | | CCCTGGAGGCGTGGGTTCGAATCCCACTCCTGACAA |
| 291 | Gly_TCC_chr1:161500902-161500974 (+) | CGCGTTGGTGGTATAGTGGTGAGCATAGCTGCCTTCCAAGCAGTTGAC |
| | | CCGGGTTCGATTCCCGGCCAACGCA |
| 292 | Asn_GTT_chr1:161510030-161510104 (+) | CGTCTCTGTGGCGCAATCGGTTAGCGCGTTCGGCTGTTAACCGAAAGG |
| | | TTGGTGGTTCGATCCCACCCAGGGACG |
| 293 | Glu_TTC_chr1:161582507-161582579 (+) | CGCGTTGGTGGTGTAGTGGTGAGCACAGCTGCCTTTCAAGCAGTTAAC |
| | | GCGGGTTCGATTCCCGGGTAACGAA |
| 294 | Pro_CGG_chr1:167683961-167684033 (+) | CGGCTCGTTGGTCTAGGGGTATGATTCTCGCTTCGGGTGCGAGAGGTC |
| | | CCGGGTTCAAATCCCGGACGAGCCC |
| 295 | Pro_AGG_chr1:167684724-167684796 (−) | GGCTCGTTGGTCTAGGGGTATGATTCTCGCTTAGGGTGCGAGAGGTCC |
| | | CGGGTTCAAATCCCGGACGAGCCCT |
| 296 | Lys_TTT_chr1:204475654-204475727 (+) | CGCCCGGATAGCTCAGTCGGTAGAGCATCAGACTTTTAATCTGAGGGT |
| | | CCAGGGTTCAAGTCCCTGTTCGGGCG |
| 297 | Lys_TTT_chr1:204476157-204476230 (−) | GCCCGGATAGCTCAGTCGGTAGAGCATCAGACTTTTAATCTGAGGGTC |
| | | CAGGGTTCAAGTCCCTGTTCGGGCGT |
| 298 | Leu_CAA_chr1:249168053-249168159 (+) | TGTCAGGATGGCCGAGTGGTCTAAGGCGCCAGACTCAAGGTAAGCACC |
| | | TTGCCTGCGGGCTTTCTGGTCTCCGGATGGAGGCGTGGGTTCGAATCCC |
| 299 | Glu_CTC_chr1:249168446-249168518 (+) | TTCCCTGGTGGTCTAGTGGTTAGGATTCGGCGCTCTCACCGCCGCGGCC |
| | | CGGGTTCGATTCCCGGTCAGGAAA |
| 300 | Tyr_GTA_chr2:27273649-27273738 (+) | GCCTTCGATAGCTCAGTTGGTAGAGCGGAGGACTGTAGTGGATAGGGC |
| | | GTGGCAATCCTTAGGTCGCTGGTTCGATTCCGGCTCGAAGGA |
| 301 | Ala_AGC_chr2:27274081-27274154 (+) | CGGGGGATTAGCTCAAATGGTAGAGCGCTCGCTTAGCATGCGAGAGGT |
| | | AGCGGGATCGATGCCCGCATCCTCCA |
| 302 | Ile_TAT_chr2:43037675-43037768 (+) | AGCTCCAGTGGCGCAATCGGTTAGCGCGCGGTACTTATACAGCAGTAC |
| | | ATGCAGAGCAATGCCGAGGTTGTGAGTTCGAGCCTCACCTGGAGCA |
| 303 | Gly_CCC_chr2:70476122-70476193 (−) | GCGCCGCTGGTGTAGTGGTATCATGCAAGATTCCCATTCTTGCGACCCG |
| | | GGTTCGATTCCCGGGCGGCGCAT |
| 304 | Glu_TTC_chr2:131094700-131094772 (−) | TCCCATATGGTCTAGCGGTTAGGATTCCTGGTTTTCACCCAGGTGGCCC |
| | | GGGTTCGACTCCCGGTATGGGAAC |
| 305 | Ala_CGC_chr2:157257280-157257352 (+) | GGGGGATGTAGCTCAGTGGTAGAGCGCGCGCTTCGCATGTGTGAGGTC |
| | | CCGGGTTCAATCCCCGGCATCTCCA |
| 306 | Gly_GCC_chr2:157257658-157257729 (−) | GCATTGGTGGTTCAGTGGTAGAATTCTCGCCTGCCACGCGGGAGGCCC |
| | | GGGTTCGATTCCCGGCCAATGCAA |
| 307 | Arg_ACG_chr3:45730490-45730563 (−) | GGGCCAGTGGCGCAATGGATAACGCGTCTGACTACGGATCAGAAGATT |
| | | CTAGGTTCGACTCCTGGCTGGCTCGC |
| 308 | Val_AAC_chr3:169490017-169490090 (+) | GGTTTCCGTAGTGTAGTGGTTATCACGTTCGCCTAACACGCGAAAGGT |
| | | CCCCGGTTCGAAACCGGGCGGAAACA |
| 309 | Val_AAC_chr5:180596609-180596682 (+) | AGTTTCCGTAGTGTAGTGGTTATCACGTTCGCCTAACACGCGAAAGGT |
| | | CCCCGGTTCGAAACCGGGCGGAAACA |
| 310 | Leu_AAG_chr5:180614700-180614782 (+) | AGGTAGCGTGGCCGAGCGGTCTAAGGCGCTGGATTAAGGCTCCAGTCT |
| | | CTTCGGGGGCGTGGGTTCGAATCCCACCGCTGCCA |
| 311 | Val_AAC_chr5:180615415-180615488 (−) | GTTTCCGTAGTGTAGTGGTCATCACGTTCGCCTAACACGCGAAAGGTC |
| | | CCCGGTTCGAAACCGGGCGGAAACAT |
| 312 | Pro_TGG_chr5:180615853-180615925 (−) | GGCTCGTTGGTCTAGGGGTATGATTCTCGCTTTGGGTGCGAGAGGTCCC |
| | | GGGTTCAAATCCCGGACGAGCCCA |
| 313 | Thr_TGT_chr5:180618686-180618758 (−) | GGCTCCATAGCTCAGGGGTTAGAGCACTGGTCTTGTAAACCAGGGTCG |
| | | CGAGTTCAAATCTCGCTGGGGCCTG |
| 314 | Ala_TGC_chr5:180633867-180633939 (+) | TGGGGATGTAGCTCAGTGGTAGAGCGCATGCTTTGCATGTATGAGGCC |
| | | CCGGGTTCGATCCCCGGCATCTCCA |
| 315 | Lys_CTT_chr5:180634754-180634827 (+) | CGCCCGGCTAGCTCAGTCGGTAGAGCATGAGACTCTTAATCTCAGGGT |
| | | CGTGGGTTCGAGCCCCACGTTGGGCG |
| 316 | Val_AAC_chr5:180645269-180645342 (−) | GTTTCCGTAGTGTAGTGGTTATCACGTTCGCCTAACACGCGAAAGGTCC |
| | | CCGGTTCGAAACCGGGCGGAAACAA |
| 317 | Lys_CTT_chr5:180648978-180649051 (−) | GCCCGGCTAGCTCAGTCGGTAGAGCATGAGACTCTTAATCTCAGGGTC |
| | | GTGGGTTCGAGCCCCACGTTGGGCGT |
| 318 | Val_CAC_chr5:180649394-180649467 (−) | GTTTCCGTAGTGTAGTGGTTATCACGTTCGCCTCACACGCGAAAGGTCC |
| | | CCGGTTCGAAACCGGGCGGAAACAC |
| 319 | Met_CAT_chr6:26286753-26286825 (+) | CAGCAGAGTGGCGCAGCGGAAGCGTGCTGGGCCCATAACCCAGAGGT |
| | | CGATGGATCGAAACCATCCTCTGCTA |
| 320 | Ser_GCT_chr6:26305717-26305801 (−) | GGAGAGGCCTGGCCGAGTGGTTAAGGCGATGGACTGCTAATCCATTGT |
| | | GCTCTGCACGCGTGGGTTCGAATCCCATCCTCGTCGC |
| 321 | Gln_TTG_chr6:26311423-26311495 (−) | GGCCCCATGGTGTAATGGTTAGCACTCTGGACTTTGAATCCAGCGATC |
| | | CGAGTTCAAATCTCGGTGGGACCTG |
| 322 | Gln_TTG_chr6:26311974-26312046 (−) | GGCCCCATGGTGTAATGGTTAGCACTCTGGACTTTGAATCCAGCGATC |
| | | CGAGTTCAAATCTCGGTGGGACCTA |
| 323 | Ser_TGA_chr6:26312823-26312905 (−) | GTAGTCGTGGCCGAGTGGTTAAGGCGATGGACTTGAAATCCATTGGGG |
| | | TCTCCCCGCGCAGGTTCGAATCCTGCCGACTACGG |
| 324 | Met_CAT_chr6:26313351-26313423 (−) | AGCAGAGTGGCGCAGCGGAAGCGTGCTGGGCCCATAACCCAGAGGTC |
| | | GATGGATCGAAACCATCCTCTGCTAT |
| 325 | Arg_TCG_chr6:26323045-26323118 (+) | GGACCACGTGGCCTAATGGATAAGGCGTCTGACTTCGGATCAGAAGAT |
| | | TGAGGGTTCGAATCCCTCCGTGGTTA |
| 326 | Ser_AGA_chr6:26327816-26327898 (+) | TGTAGTCGTGGCCGAGTGGTTAAGGCGATGGACTAGAAATCCATTGGG |
| | | GTCTCCCCGCGCAGGTTCGAATCCTGCCGACTACG |
| 327 | Met_CAT_chr6:26330528-26330600 (−) | AGCAGAGTGGCGCAGCGGAAGCGTGCTGGGCCCATAACCCAGAGGTC |
| | | GATGGATCGAAACCATCCTCTGCTAG |
| 328 | Leu_CAG_chr6:26521435-26521518 (+) | CGTCAGGATGGCCGAGCGGTCTAAGGCGCTGCGTTCAGGTCGCAGTCT |
| | | CCCCTGGAGGCGTGGGTTCGAATCCCACTCCTGACA |
| 329 | Thr_AGT_chr6:26533144-26533218 (−) | GGCTCCGTGGCTTAGCTGGTTAAAGCGCCTGTCTAGTAAACAGGAGAT |
| | | CCTGGGTTCGAATCCCAGCGGGGCCTG |
| 330 | Arg_ACG_chr6:26537725-26537798 (+) | AGGGCCAGTGGCGCAATGGATAACGCGTCTGACTACGGATCAGAAGA |
| | | TTCCAGGTTCGACTCCTGGCTGGCTCG |
| 331 | Val_CAC_chr6:26538281-26538354 (+) | GGTTTCCGTAGTGTAGTGGTTATCACGTTCGCCTCACACGCGAAAGGTC |
| | | CCCGGTTCGAAACCGGGCGGAAACA |
| 332 | Ala_CGC_chr6:26553730-26553802 (+) | AGGGGATGTAGCTCAGTGGTAGAGCGCATGCTTCGCATGTATGAGGTC |
| | | CCGGGTTCGATCCCCGGCATCTCCA |
| 333 | Ile_AAT_chr6:26554349-26554423 (+) | TGGCCGGTTAGCTCAGTTGGTTAGAGCGTGGTGCTAATAACGCCAAGG |
| | | TCGCGGGTTCGATCCCCGTACGGGCCA |
| 334 | Pro_AGG_chr6:26555497-26555569 (+) | CGGCTCGTTGGTCTAGGGGTATGATTCTCGCTTAGGGTGCGAGAGGTC |
| | | CCGGGTTCAAATCCCGGACGAGCCC |
| 335 | Lys_CTT_chr6:26556773-26556846 (+) | AGCCCGGCTAGCTCAGTCGGTAGAGCATGAGACTCTTAATCTCAGGGT |
| | | CGTGGGTTCGAGCCCCACGTTGGGCG |
| 336 | Tyr_GTA_chr6:26569085-26569176 (+) | TCCTTCGATAGCTCAGTTGGTAGAGCGGAGGACTGTAGTTGGCTGTGT |
| | | CCTTAGACATCCTTAGGTCGCTGGTTCGAATCCGGCTCGAAGGA |
| 337 | Ala_AGC_chr6:26572091-26572164 (−) | GGGGAATTAGCTCAAATGGTAGAGCGCTCGCTTAGCATGCGAGAGGTA |
| | | GCGGGATCGATGCCCGCATTCTCCAG |
| 338 | Met_CAT_chr6:26766443-26766516 (+) | CGCCCTCTTAGCGCAGCGGGCAGCGCGTCAGTCTCATAATCTGAAGGT |
| | | CCTGAGTTCGAGCCTCAGAGAGGGCA |
| 339 | Ile_TAT_chr6:26988124-26988218 (+) | TGCTCCAGTGGCGCAATCGGTTAGCGCGCGGTACTTATATGGCAGTAT |
| | | GTGTGCGAGTGATGCCGAGGTTGTGAGTTCGAGCCTCACCTGGAGCA |
| 340 | His_GTG_chr6:27125905-27125977 (+) | TGCCGTGATCGTATAGTGGTTAGTACTCTGCGTTGTGGCCGCAGCAACC |
| | | TCGGTTCGAATCCGAGTCACGGCA |
| 341 | Ile_AAT_chr6:27144993-27145067 (−) | GGCCGGTTAGCTCAGTTGGTTAGAGCGTGGTGCTAATAACGCCAAGGT |
| | | CGCGGGTTCGATCCCCGTACGGGCCAC |
| 342 | Val_AAC_chr6:27203287-27203360 (+) | AGTTTCCGTAGTGTAGTGGTTATCACGTTTGCCTAACACGCGAAAGGTC |
| | | CCCGGTTCGAAACCGGGCAGAAACA |
| 343 | Val_CAC_chr6:27248048-27248121 (−) | GCTTCTGTAGTGTAGTGGTTATCACGTTCGCCTCACACGCGAAAGGTCC |
| | | CCGGTTCGAAACCGGGCAGAAGCAA |
| 344 | Asp_GTC_chr6:27447452-27447524 (+) | TTCCTCGTTAGTATAGTGGTGAGTATCCCCGCCTGTCACGCGGGAGACC |
| | | GGGGTTCGATTCCCCGACGGGGAG |
| 345 | Ser_TGA_chr6:27473606-27473688 (−) | GTAGTCGTGGCCGAGTGGTTAAGGCGATGGACTTGAAATCCATTGGGG |
| | | TTTCCCCGCGCAGGTTCGAATCCTGTCGGCTACGG |
| 346 | Gln_CTG_chr6:27487307-27487379 (+) | AGGTTCCATGGTGTAATGGTTAGCACTCTGGACTCTGAATCCAGCGAT |
| | | CCGAGTTCAAATCTCGGTGGAACCT |
| 347 | Asp_GTC_chr6:27551235-27551307 (−) | TCCTCGTTAGTATAGTGGTGAGTGTCCCCGTCTGTCACGCGGGAGACC |
| | | GGGGTTCGATTCCCCGACGGGGAGA |
| 348 | Val_AAC_chr6:27618706-27618779 (−) | GTTTCCGTAGTGTAGTGGTTATCACGTTCGCCTAACACGCGAAAGGTCC |
| | | CTGGATCAAAACCAGGCGGAAACAA |
| 349 | Ile_AAT_chr6:27655966-27656040 (+) | CGGCCGGTTAGCTCAGTTGGTTAGAGCGTGGTGCTAATAACGCCAAGG |
| | | TCGCGGGTTCGATCCCCGTACTGGCCA |
| 350 | Gln_CTG_chr6:27759134-27759206 (−) | GGCCCCATGGTGTAATGGTCAGCACTCTGGACTCTGAATCCAGCGATC |
| | | CGAGTTCAAATCTCGGTGGGACCCA |
| 351 | Gln_TTG_chr6:27763639-27763711 (−) | GGCCCCATGGTGTAATGGTTAGCACTCTGGACTTTGAATCCAGCGATC |
| | | CGAGTTCAAATCTCGGTGGGACCTT |
| 352 | Ala_AGC_chr6:28574932-28575004 (+) | TGGGGGTGTAGCTCAGTGGTAGAGCGCGTGCTTAGCATGTACGAGGTC |
| | | CCGGGTTCAATCCCCGGCACCTCCA |
| 353 | Ala_AGC_chr6:28626013-28626085 (−) | GGGGATGTAGCTCAGTGGTAGAGCGCATGCTTAGCATGCATGAGGTCC |
| | | CGGGTTCGATCCCCAGCATCTCCAG |
| 354 | Ala_CGC_chr6:28697091-28697163 (+) | AGGGGGTGTAGCTCAGTGGTAGAGCGCGTGCTTCGCATGTACGAGGCC |
| | | CCGGGTTCGACCCCCGGCTCCTCCA |
| 355 | Ala_AGC_chr6:28806220-28806292 (−) | GGGGGTGTAGCTCAGTGGTAGAGCGCGTGCTTAGCATGCACGAGGCCC |
| | | CGGGTTCAATCCCCGGCACCTCCAT |
| 356 | Ala_AGC_chr6:28831461-28831533 (−) | GGGGGTGTAGCTCAGTGGTAGAGCGCGTGCTTAGCATGCACGAGGCCC |
| | | CGGGTTCAATCCCCGGCACCTCCAG |
| 357 | Leu_CAA_chr6:28863999-28864105 (−) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| 358 | Leu_CAA_chr6:28908829-28908934 (+) | TGTCAGGATGGCCGAGTGGTCTAAGGCGCCAGACTCAAGCTTGGCTTC |
| | | CTCGTGTTGAGGATTCTGGTCTCCAATGGAGGCGTGGGTTCGAATCCC |
| 359 | Gln_CTG_chr6:28909377-28909449 (−) | GGTTCCATGGTGTAATGGTTAGCACTCTGGACTCTGAATCCAGCGATCC |
| | | GAGTTCAAATCTCGGTGGAACCTT |
| 360 | Leu_AAG_chr6:28911398-28911480 (−) | GGTAGCGTGGCCGAGCGGTCTAAGGCGCTGGATTAAGGCTCCAGTCTC |
| | | TTCGGGGGCGTGGGTTCGAATCCCACCGCTGCCAG |
| 361 | Met_CAT_chr6:28912351-28912424 (+) | TGCCTCCTTAGCGCAGTAGGCAGCGCGTCAGTCTCATAATCTGAAGGT |
| | | CCTGAGTTCGAACCTCAGAGGGGGCA |
| 362 | Lys_TTT_chr6:28918805-28918878 (+) | AGCCCGGATAGCTCAGTCGGTAGAGCATCAGACTTTTAATCTGAGGGT |
| | | CCAGGGTTCAAGTCCCTGTTCGGGCG |
| 363 | Met_CAT_chr6:28921041-28921114 (−) | GCCTCCTTAGCGCAGTAGGCAGCGCGTCAGTCTCATAATCTGAAGGTC |
| | | CTGAGTTCGAACCTCAGAGGGGGCAG |
| 364 | Glu_CTC_chr6:28949975-28950047 (+) | TTCCCTGGTGGTCTAGTGGTTAGGATTCGGCGCTCTCACCGCCGCGGCC |
| | | CGGGTTCGATTCCCGGTCAGGGAA |
| 365 | Leu_TAA_chr6:144537683-144537766 (+) | CACCAGGATGGCCGAGTGGTTAAGGCGTTGGACTTAAGATCCAATGGA |
| | | CATATGTCCGCGTGGGTTCGAACCCCACTCCTGGTA |
| 366 | Pro_AGG_chr7:128423503-128423575 (+) | TGGCTCGTTGGTCTAGGGGTATGATTCTCGCTTAGGGTGCGAGAGGTC |
| | | CCGGGTTCAAATCCCGGACGAGCCC |
| 367 | Arg_CCT_chr7:139025445-139025518 (+) | AGCCCCAGTGGCCTAATGGATAAGGCATTGGCCTCCTAAGCCAGGGAT |
| | | TGTGGGTTCGAGTCCCATCTGGGGTG |
| 368 | Cys_GCA_chr7:149388271-149388343 (−) | GGGGATATAGCTCAGGGGTAGAGCATTTGACTGCAGATCAAGAGGTCC |
| | | CCGGTTCAAATCCGGGTGCCCCCCC |
| 369 | Tyr_GTA_chr8:67025601-67025694 (+) | CCCTTCGATAGCTCAGCTGGTAGAGCGGAGGACTGTAGCTACTTCCTC |
| | | AGCAGGAGACATCCTTAGGTCGCTGGTTCGATTCCGGCTCGAAGGA |
| 370 | Tyr_GTA_chr8:67026222-67026311 (+) | CCCTTCGATAGCTCAGCTGGTAGAGCGGAGGACTGTAGGCGCGCGCCC |
| | | GTGGCCATCCTTAGGTCGCTGGTTCGATTCCGGCTCGAAGGA |
| 371 | Ala_AGC_chr8:67026423-67026496 (+) | TGGGGGATTAGCTCAAATGGTAGAGCGCTCGCTTAGCATGCGAGAGGT |
| | | AGCGGGATCGATGCCCGCATCCTCCA |
| 372 | Ser_AGA_chr8:96281884-96281966 (−) | GTAGTCGTGGCCGAGTGGTTAAGGCGATGGACTAGAAATCCATTGGGG |
| | | TCTCCCCGCGCAGGTTCGAATCCTGCCGACTACGG |
| 373 | Met_CAT_chr8:124169469-124169542 (−) | GCCTCGTTAGCGCAGTAGGTAGCGCGTCAGTCTCATAATCTGAAGGTC |
| | | GTGAGTTCGATCCTCACACGGGGCAC |
| 374 | Arg_TCT_chr9:131102354-131102445 (−) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCGA |
| 375 | Asn_GTT_chr10:22518437-22518511 (−) | GTCTCTGTGGCGCAATCGGTTAGCGCGTTCGGCTGTTAACCGAAAGGT |
| | | TGGTGGTTCGAGCCCACCCAGGGACGC |
| 376 | Ser_TGA_chr10:69524260-69524342 (+) | GGCAGCGATGGCCGAGTGGTTAAGGCGTTGGACTTGAAATCCAATGGG |
| | | GTCTCCCCGCGCAGGTTCGAACCCTGCTCGCTGCG |
| 377 | Val_TAC_chr11:59318101-59318174 (−) | GGTTCCATAGTGTAGTGGTTATCACGTCTGCTTTACACGCAGAAGGTCC |
| | | TGGGTTCGAGCCCCAGTGGAACCAT |
| 378 | Val_TAC_chr11:59318459-59318532 (−) | GGTTCCATAGTGTAGCGGTTATCACGTCTGCTTTACACGCAGAAGGTCC |
| | | TGGGTTCGAGCCCCAGTGGAACCAC |
| 379 | Arg_TCT_chr11:59318766-59318852 (+) | TGGCTCTGTGGCGCAATGGATAGCGCATTGGACTTCTAGATAGTTAGA |
| | | GAAATTCAAAGGTTGTGGGTTCGAGTCCCACCAGAGTCG |
| 380 | Leu_TAA_chr11:59319227-59319310 (+) | TACCAGAATGGCCGAGTGGTTAAGGCGTTGGACTTAAGATCCAATGGA |
| | | TTCATATCCGCGTGGGTTCGAACCCCACTTCTGGTA |
| 381 | Lys_TTT_chr11:59323901-59323974 (+) | GGCCCGGATAGCTCAGTCGGTAGAGCATCAGACTTTTAATCTGAGGGT |
| | | CCGGGGTTCAAGTCCCTGTTCGGGCG |
| 382 | Phe_GAA_chr11:59324969-59325042 (−) | GCCGAAATAGCTCAGTTGGGAGAGCGTTAGACTGAAGATCTAAAGGTC |
| | | CCTGGTTCGATCCCGGGTTTCGGCAG |
| 383 | Lys_TTT_chr11:59327807-59327880 (−) | GCCCGGATAGCTCAGTCGGTAGAGCATCAGACTTTTAATCTGAGGGTC |
| | | CAGGGTTCAAGTCCCTGTTCGGGCGG |
| 384 | Phe_GAA_chr11:59333852-59333925 (−) | GCCGAAATAGCTCAGTTGGGAGAGCGTTAGACTGAAGATCTAAAGGTC |
| | | CCTGGTTCAATCCCGGGTTTCGGCAG |
| 385 | Ser_GCT_chr11:66115590-66115672 (+) | GGACGAGGTGGCCGAGTGGTTAAGGCGATGGACTGCTAATCCATTGTG |
| | | CTTTGCACGCGTGGGTTCGAATCCCATCCTCGTCG |
| 386 | Pro_TGG_chr11:75946868-75946940 (−) | GGCTCGTTGGTCTAGGGGTATGATTCTCGGTTTGGGTCCGAGAGGTCCC |
| | | GGGTTCAAATCCCGGACGAGCCCC |
| 387 | Ser_CGA_chr12:56584147-56584229 (+) | AGTCACGGTGGCCGAGTGGTTAAGGCGTTGGACTCGAAATCCAATGGG |
| | | GTTTCCCCGCACAGGTTCGAATCCTGTTCGTGACG |
| 388 | Asp_GTC_chr12:98897280-98897352 (+) | CTCCTCGTTAGTATAGTGGTTAGTATCCCCGCCTGTCACGCGGGAGACC |
| | | GGGGTTCAATTCCCCGACGGGGAG |
| 389 | Trp_CCA_chr12:98898029-98898101 (+) | GGACCTCGTGGCGCAACGGTAGCGCGTCTGACTCCAGATCAGAAGGCT |
| | | GCGTGTTCGAATCACGTCGGGGTCA |
| 390 | Ala_TGC_chr12:125406300-125406372 (−) | GGGGATGTAGCTCAGTGGTAGAGCGCATGCTTTGCATGTATGAGGCCC |
| | | CGGGTTCGATCCCCGGCATCTCCAT |
| 391 | Phe_GAA_chr12:125412388-125412461 (−) | GCCGAAATAGCTCAGTTGGGAGAGCGTTAGACTGAAGATCTAAAGGTC |
| | | CCTGGTTCGATCCCGGGTTTCGGCAG |
| 392 | Ala_TGC_chr12:125424511-125424583 (+) | AGGGGATGTAGCTCAGTGGTAGAGCGCATGCTTTGCACGTATGAGGCC |
| | | CCGGGTTCAATCCCCGGCATCTCCA |
| 393 | Asn_GTT_chr13:31248100-31248174 (−) | GTCTCTGTGGCGCAATCGGTTAGCGCGTTCGGCTGTTAACCGAAAGGT |
| | | TGGTGGTTCGAGCCCACCCAGGGACGG |
| 394 | Glu_TTC_chr13:45492061-45492133 (−) | TCCCACATGGTCTAGCGGTTAGGATTCCTGGTTTTCACCCAGGCGGCCC |
| | | GGGTTCGACTCCCGGTGTGGGAAC |
| 395 | Thr_TGT_chr14:21081948-21082021 (−) | GGCTCCATAGCTCAGGGGTTAGAGCGCTGGTCTTGTAAACCAGGGGTC |
| | | GCGAGTTCAATTCTCGCTGGGGCCTG |
| 396 | Leu_TAG_chr14:21093528-21093610 (+) | TGGTAGTGTGGCCGAGCGGTCTAAGGCGCTGGATTTAGGCTCCAGTCT |
| | | CTTCGGGGGCGTGGGTTCGAATCCCACCACTGCCA |
| 397 | Thr_TGT_chr14:21099318-21099391 (−) | GGCTCCATAGCTCAGGGGTTAGAGCACTGGTCTTGTAAACCAGGGGTC |
| | | GCGAGTTCAAATCTCGCTGGGGCCTC |
| 398 | Pro_TGG_chr14:21101164-21101236 (+) | TGGCTCGTTGGTCTAGTGGTATGATTCTCGCTTTGGGTGCGAGAGGTCC |
| | | CGGGTTCAAATCCCGGACGAGCCC |
| 399 | Tyr_GTA_chr14:21131350-21131444 (−) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGAA |
| 400 | Thr_TGT_chr14:21149848-21149921 (+) | AGGCCCTATAGCTCAGGGGTTAGAGCACTGGTCTTGTAAACCAGGGGT |
| | | CGCGAGTTCAAATCTCGCTGGGGCCT |
| 401 | Tyr_GTA_chr14:21151431-21151520 (+) | TCCTTCGATAGCTCAGCTGGTAGAGCGGAGGACTGTAGTACTTAATGT |
| | | GTGGTCATCCTTAGGTCGCTGGTTCGATTCCGGCTCGAAGGA |
| 402 | Pro_TGG_chr14:21152174-21152246 (+) | TGGCTCGTTGGTCTAGGGGTATGATTCTCGCTTTGGGTGCGAGAGGTCC |
| | | CGGGTTCAAATCCCGGACGAGCCC |
| 403 | Lys_CTT_chr14:58706612-58706685 (−) | GCCCGGCTAGCTCAGTCGGTAGAGCATGGGACTCTTAATCCCAGGGTC |
| | | GTGGGTTCGAGCCCCACGTTGGGCGC |
| 404 | Ile_AAT_chr14:102783428-102783502 (+) | CGGCCGGTTAGCTCAGTTGGTTAGAGCGTGGTGCTAATAACGCCAAGG |
| | | TCGCGGGTTCGATCCCCGTACGGGCCA |
| 405 | Glu_TTC_chr15:26327380-26327452 (−) | TCCCACATGGTCTAGCGGTTAGGATTCCTGGTTTTCACCCAGGCGGCCC |
| | | GGGTTCGACTCCCGGTGTGGGAAT |
| 406 | Ser_GCT_chr15:40886022-40886104 (−) | GACGAGGTGGCCGAGTGGTTAAGGCGATGGACTGCTAATCCATTGTGC |
| | | TCTGCACGCGTGGGTTCGAATCCCATCCTCGTCGA |
| 407 | His_GTG_chr15:45490803-45490875 (−) | GCCGTGATCGTATAGTGGTTAGTACTCTGCGTTGTGGCCGCAGCAACCT |
| | | CGGTTCGAATCCGAGTCACGGCAT |
| 408 | His_GTG_chr15:45493348-45493420 (+) | CGCCGTGATCGTATAGTGGTTAGTACTCTGCGTTGTGGCCGCAGCAAC |
| | | CTCGGTTCGAATCCGAGTCACGGCA |
| 409 | Gln_CTG_chr15:66161399-66161471 (−) | GGTTCCATGGTGTAATGGTTAGCACTCTGGACTCTGAATCCAGCGATCC |
| | | GAGTTCAAATCTCGGTGGAACCTG |
| 410 | Lys_CTT_chr15:79152903-79152976 (+) | TGCCCGGCTAGCTCAGTCGGTAGAGCATGGGACTCTTAATCCCAGGGT |
| | | CGTGGGTTCGAGCCCCACGTTGGGCG |
| 411 | Arg_TCG_chr15:89878303-89878376 (+) | GGGCCGCGTGGCCTAATGGATAAGGCGTCTGACTTCGGATCAGAAGAT |
| | | TGCAGGTTCGAGTCCTGCCGCGGTCG |
| 412 | Gly_CCC_chr16:686735-686806 (−) | GCGCCGCTGGTGTAGTGGTATCATGCAAGATTCCCATTCTTGCGACCCG |
| | | GGTTCGATTCCCGGGCGGCGCAC |
| 413 | Arg_CCG_chr16:3200674-3200747 (+) | GGGCCGCGTGGCCTAATGGATAAGGCGTCTGATTCCGGATCAGAAGAT |
| | | TGAGGGTTCGAGTCCCTTCGTGGTCG |
| 414 | Arg_CCT_chr16:3202900-3202973 (+) | CGCCCCGGTGGCCTAATGGATAAGGCATTGGCCTCCTAAGCCAGGGAT |
| | | TGTGGGTTCGAGTCCCACCCGGGGTA |
| 415 | Lys_CTT_chr16:3207405-3207478 (−) | GCCCGGCTAGCTCAGTCGGTAGAGCATGAGACCCTTAATCTCAGGGTC |
| | | GTGGGTTCGAGCCCCACGTTGGGCGT |
| 416 | Thr_CGT_chr16:14379749-14379821 (+) | AGGCGCGGTGGCCAAGTGGTAAGGCGTCGGTCTCGTAAACCGAAGATC |
| | | ACGGGTTCGAACCCCGTCCGTGCCT |
| 417 | Leu_TAG_chr16:22207031-22207113 (−) | GGTAGCGTGGCCGAGTGGTCTAAGGCGCTGGATTTAGGCTCCAGTCAT |
| | | TTCGATGGCGTGGGTTCGAATCCCACCGCTGCCAC |
| 418 | Leu_AAG_chr16:22308460-22308542 (+) | GGGTAGCGTGGCCGAGCGGTCTAAGGCGCTGGATTAAGGCTCCAGTCT |
| | | CTTCGGGGGCGTGGGTTCGAATCCCACCGCTGCCA |
| 419 | Leu_CAG_chr16:57333862-57333945 (+) | AGTCAGGATGGCCGAGCGGTCTAAGGCGCTGCGTTCAGGTCGCAGTCT |
| | | CCCCTGGAGGCGTGGGTTCGAATCCCACTTCTGACA |
| 420 | Leu_CAG_chr16:57334391-57334474 (−) | GTCAGGATGGCCGAGCGGTCTAAGGCGCTGCGTTCAGGTCGCAGTCTC |
| | | CCCTGGAGGCGTGGGTTCGAATCCCACTTCTGACAG |
| 421 | Met_CAT_chr16:87417627-87417700 (−) | GCCTCGTTAGCGCAGTAGGCAGCGCGTCAGTCTCATAATCTGAAGGTC |
| | | GTGAGTTCGAGCCTCACACGGGGCAG |
| 422 | Leu_TAG_chr17:8023631-8023713 (−) | GGTAGCGTGGCCGAGCGGTCTAAGGCGCTGGATTTAGGCTCCAGTCTC |
| | | TTCGGAGGCGTGGGTTCGAATCCCACCGCTGCCAG |
| 423 | Arg_TCT_chr17:8024242-8024330 (+) | TGGCTCTGTGGCGCAATGGATAGCGCATTGGACTTCTAGTGACGAATA |
| | | GAGCAATTCAAAGGTTGTGGGTTCGAATCCCACCAGAGTCG |
| 424 | Gly_GCC_chr17:8029063-8029134 (+) | CGCATTGGTGGTTCAGTGGTAGAATTCTCGCCTGCCACGCGGGAGGCC |
| | | CGGGTTCGATTCCCGGCCAATGCA |
| 425 | Ser_CGA_chr17:8042198-8042280 (−) | GCTGTGATGGCCGAGTGGTTAAGGCGTTGGACTCGAAATCCAATGGGG |
| | | TCTCCCCGCGCAGGTTCGAATCCTGCTCACAGCGT |
| 426 | Thr_AGT_chr17:8042769-8042843 (−) | GGCGCCGTGGCTTAGCTGGTTAAAGCGCCTGTCTAGTAAACAGGAGAT |
| | | CCTGGGTTCGAATCCCAGCGGTGCCTG |
| 427 | Trp_CCA_chr17:8089675-8089747 (+) | CGACCTCGTGGCGCAACGGTAGCGCGTCTGACTCCAGATCAGAAGGTT |
| | | GCGTGTTCAAATCACGTCGGGGTCA |
| 428 | Ser_GCT_chr17:8090183-8090265 (+) | AGACGAGGTGGCCGAGTGGTTAAGGCGATGGACTGCTAATCCATTGTG |
| | | CTCTGCACGCGTGGGTTCGAATCCCATCCTCGTCG |
| 429 | Thr_AGT_chr17:8090477-8090551 (+) | CGGCGCCGTGGCTTAGTTGGTTAAAGCGCCTGTCTAGTAAACAGGAGA |
| | | TCCTGGGTTCGAATCCCAGCGGTGCCT |
| 430 | Trp_CCA_chr17:8124186-8124258 (−) | GGCCTCGTGGCGCAACGGTAGCGCGTCTGACTCCAGATCAGAAGGTTG |
| | | CGTGTTCAAATCACGTCGGGGTCAA |
| 431 | Gly_TCC_chr17:8124865-8124937 (+) | AGCGTTGGTGGTATAGTGGTAAGCATAGCTGCCTTCCAAGCAGTTGAC |
| | | CCGGGTTCGATTCCCGGCCAACGCA |
| 432 | Asp_GTC_chr17:8125555-8125627 (−) | TCCTCGTTAGTATAGTGGTGAGTATCCCCGCCTGTCACGCGGGAGACC |
| | | GGGGTTCGATTCCCCGACGGGGAGA |
| 433 | Pro_CGG_chr17:8126150-8126222 (−) | GGCTCGTTGGTCTAGGGGTATGATTCTCGCTTCGGGTGCGAGAGGTCC |
| | | CGGGTTCAAATCCCGGACGAGCCCT |
| 434 | Thr_AGT_chr17:8129552-8129626 (−) | GGCGCCGTGGCTTAGTTGGTTAAAGCGCCTGTCTAGTAAACAGGAGAT |
| | | CCTGGGTTCGAATCCCAGCGGTGCCTT |
| 435 | Ser_AGA_chr17:8129927-8130009 (−) | GTAGTCGTGGCCGAGTGGTTAAGGCGATGGACTAGAAATCCATTGGGG |
| | | TCTCCCCGCGCAGGTTCGAATCCTGCCGACTACGT |
| 436 | Trp_CCA_chr17:19411493-19411565 (+) | TGACCTCGTGGCGCAATGGTAGCGCGTCTGACTCCAGATCAGAAGGTT |
| | | GCGTGTTCAAGTCACGTCGGGGTCA |
| 437 | Thr_CGT_chr17:29877092-29877164 (+) | AGGCGCGGTGGCCAAGTGGTAAGGCGTCGGTCTCGTAAACCGAAGATC |
| | | GCGGGTTCGAACCCCGTCCGTGCCT |
| 438 | Cys_GCA_chr17:37023897-37023969 (+) | AGGGGGTATAGCTCAGTGGTAGAGCATTTGACTGCAGATCAAGAGGTC |
| | | CCCGGTTCAAATCCGGGTGCCCCCT |
| 439 | Cys_GCA_chr17:37025544-37025616 (−) | GGGGGTATAGCTCAGTGGTAGAGCATTTGACTGCAGATCAAGAGGTCC |
| | | CTGGTTCAAATCCGGGTGCCCCCTC |
| 440 | Cys_GCA_chr17:37309986-37310058 (−) | GGGGGTATAGCTCAGTGGTAGAGCATTTGACTGCAGATCAAGAGGTCC |
| | | CCGGTTCAAATCCGGGTGCCCCCTC |
| 441 | Gln_TTG_chr17:47269889-47269961 (+) | AGGTCCCATGGTGTAATGGTTAGCACTCTGGACTTTGAATCCAGCGAT |
| | | CCGAGTTCAAATCTCGGTGGGACCT |
| 442 | Arg_CCG_chr17:66016012-66016085 (−) | GACCCAGTGGCCTAATGGATAAGGCATCAGCCTCCGGAGCTGGGGATT |
| | | GTGGGTTCGAGTCCCATCTGGGTCGC |
| 443 | Arg_CCT_chr17:73030000-73030073 (+) | AGCCCCAGTGGCCTAATGGATAAGGCACTGGCCTCCTAAGCCAGGGAT |
| | | TGTGGGTTCGAGTCCCACCTGGGGTA |
| 444 | Arg_CCT_chr17:73030525-73030598 (−) | GCCCCAGTGGCCTAATGGATAAGGCACTGGCCTCCTAAGCCAGGGATT |
| | | GTGGGTTCGAGTCCCACCTGGGGTGT |
| 445 | Arg_TCG_chr17:73031207-73031280 (+) | AGACCGCGTGGCCTAATGGATAAGGCGTCTGACTTCGGATCAGAAGAT |
| | | TGAGGGTTCGAGTCCCTTCGTGGTCG |
| 446 | Asn_GTT_chr19:1383561-1383635 (+) | CGTCTCTGTGGCGCAATCGGTTAGCGCGTTCGGCTGTTAACCGAAAGG |
| | | TTGGTGGTTCGAGCCCACCCAGGGACG |
| 447 | Gly_TCC_chr19:4724081-4724153 (+) | GGCGTTGGTGGTATAGTGGTTAGCATAGCTGCCTTCCAAGCAGTTGAC |
| | | CCGGGTTCGATTCCCGGCCAACGCA |
| 448 | Val_CAC_chr19:4724646-4724719 (−) | GTTTCCGTAGTGTAGCGGTTATCACATTCGCCTCACACGCGAAAGGTCC |
| | | CCGGTTCGATCCCGGGCGGAAACAG |
| 449 | Thr_AGT_chr19:33667962-33668036 (+) | TGGCGCCGTGGCTTAGTTGGTTAAAGCGCCTGTCTAGTAAACAGGAGA |
| | | TCCTGGGTTCGAATCCCAGCGGTGCCT |
| 450 | Ile_TAT_chr19:39902807-39902900 (−) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCAC |
| 451 | Gly_GCC_chr21:18827106-18827177 (−) | GCATGGGTGGTTCAGTGGTAGAATTCTCGCCTGCCACGCGGGAGGCCC |
| | | GGGTTCGATTCCCGGCCCATGCAG |

### Non-Naturally Occurring Modification

A TREM, a TREM core fragment or a TREM fragment described herein comprises a non-naturally occurring modification, e.g., a modification described in any one of Tables 5-9. A non-naturally occurring modification can be made according to methods known in the art. Exemplary methods of making non-naturally occurring modifications are provided in Examples 4-7.

In an embodiment, a non-naturally occurring modification is a modification that a cell, e.g., a human cell, does not make on an endogenous tRNA.

In an embodiment, a non-naturally occurring modification is a modification that a cell, e.g., a human cell, can make on an endogenous tRNA, but wherein such modification is in a location in which it does not occur on a native tRNA. In an embodiment, the non-naturally occurring modification is in a domain, linker or arm which does not have such modification in nature. In an embodiment, the non-naturally occurring modification is at a position within a domain, linker or arm, which does not have such modification in nature. In an embodiment, the non-naturally occurring modification is on a nucleotide which does not have such modification in nature. In an embodiment, the non-naturally occurring modification is on a nucleotide at a position within a domain, linker or arm, which does not have such modification in nature.

In an embodiment, a TREM, a TREM core fragment or a TREM fragment described herein comprises a non-naturally occurring modification provided in Table 5, or a combination thereof.

TABLE 5
Exemplary non-naturally occurring modifications
Modification
7-deaza-adenosine
Nl-methyl-adenosine
N6,N6 (dimethyl)adenine
N6-cis-hydroxy-isopentenyl-adenosine
thio-adenosine
2-(amino)adenine
2-(aminopropyl)adenine
2-(methylthio) N6 (isopentenyl)adenine
2-(alkyl)adenine
6-(alkyl)adenine
6-(methyl)adenine
7-(deaza)adenine
8-(alkenyl)adenine
8-(alkynyl)adenine
8-(amino)adenine
8-(thioalkyl)adenine
8-(alkenyl)adenine
8-(alkyl)adenine
8-(alkynyl)adenine
8-(amino)adenine
8-(halo)adenine
8-(hydroxyl)adenine
8-(thioalkyl)adenine
8-(thiol)adenine
8-azido-adenosine
azaadenine
deazaadenine
N6-(methyl)adenine
N6-(isopentyl)adenine
7-deaza-8-aza-adenosine
7-methyladenine
1-deazaadenosine
2′-Fluoro-N6-Bz-deoxyadenosine
2′-OMe-2-Amino-adenosine
2′O-methyl-N6-Bz-deoxyadenosine
2′-alpha-ethynyladenosine
2-aminoadenine
2-Aminoadenosine
2-Amino-adenosine
2′-alpha-Trifluoromethyladenosine
5′-Homo-adenosine
8-Aza-adenosine
8-bromo-adenosine
8-Trifluoromethyladenosine
9-Deazaadenosine
2-aminopurine
7-deaza-2,6-diaminopurine
7-deaza-8-aza-2,6-diaminopurine
7-deaza-8-aza-2-aminopurine
2,6-diaminopurine
7-deaza-8-aza-adenine, 7-deaza-2-
aminopurine
4-methylcytidine
5-aza-cytidine
Pseudo-iso-cytidine
pyrrolo-cytidine
alpha-thio-cytidine
2-(thio)cytosine
2′-Amino-2′-deoxy-cytosine
2′-Azido-2′-deoxy-cytosine
2′-Deoxy-2′-alpha-aminocytidine
2′-Deoxy-2′-alpha-azidocytidine
3 (deaza) 5 (aza)cytosine
3 (methyl)cytosine
3-(alkyl)cytosine
3-(deaza) 5 (aza)cytosine
3-(methyl)cytidine
4,2′-O-dimethylcytidine
5 (halo)cytosine
5 (methyl)cytosine
5 (propynyl)cytosine
2′-Fluor-N4-Bz-cytidine
2′-Fluoro-N4-Acetyl-cytidine
2′-O-Methyl-N4-Acetyl-cytidine
2′-O-methyl-N4-Bz-cytidine
2′-a-Ethynylcytidine
2′-a-Trifluoromethylcytidine
2′-b-Ethynylcytidine
2′-b-Trifluoromethylcytidine
2′-Deoxy-2′,2′-difluorocytidine
2′-Deoxy-2′-alpha-mercaptocytidine
2′-Deoxy-2′-alpha-thiomethoxycytidine
2′-Deoxy-2′-betab-aminocytidine
2′-Deoxy-2′-beta-azidocytidine
2′-Deoxy-2′-beta-bromocytidine
2′-Deoxy-2′-beta-chlorocytidine
2′-Deoxy-2′-beta-fluorocytidine
2′-Deoxy-2′-beta-iodocytidine
2′-Deoxy-2′-beta-mercaptocytidine
2′-Deoxy-2′-beta-thiomethoxycytidine
TP
2′-O-Methyl-5-(1-propynyl)cytidine
3′-Ethynylcytidine
4′-Azidocytidine
4′-Carbocyclic cytidine
4′-Ethynylcytidine
5-(1-Propynyl)ara-cytidine
5-(2-Chloro-phenyl)-2-thiocytidine
5-(4-Amino-phenyl)-2-thiocytidine
5-Aminoallyl-cytosine
5-Cyanocytidine
5-Ethynylara-cytidine
8-(thioalkyl)guanine
8-(alkenyl)guanine
8-(alkyl)guanine
8-(alkynyl)guanine
8-(amino)guanine
8-(halo)guanine
8-(hydroxyl)guanine
8-(thioalkyl)guanine
8-(thiol)guanine
azaguanine
deaza guanine
N (methyl)guanine
N-(methyl)guanine
l-methyl-6-thio-guanosine
6-methoxy-guanosine
6-thio-7-deaza-8-aza-guanosine
6-thio-7-deaza-guanosine
6-thio-7-methyl-guanosine
7-deaza-8-aza-guanosine
7-methyl-8-oxo-guanosine
N2,N2-dimethyl-6-thio-guanosine
N2-methyl-6-thio-guanosine
1-Me-guanosine
2′Fluoro-N2-isobutyl-guanosine
2′O-methyl-N2-isobutyl-guanosine
2′-alpha-Ethynylguanosine
2′-alpha-Trifluoromethylguanosine
2′-beta-Ethynylguanosine
2′-beta-Trifluoromethylguanosine
2′-Deoxy-2′,2′-difluoroguanosine
2′-Deoxy-2′-alpha-mercaptoguanosine
(dithio)pseudouracil
1-(aminocarbonylethylenyl)-4
(thio)pseudouracil
1-(aminocarbonylethylenyl)-pseudouracil
1-substituted 2-(thio)-pseudouracil
1-substituted 2,4-(dithio)pseudouracil
1-substituted 4 (thio)pseudouracil
1-substituted pseudouracil
1-(aminoalkylamino-carbonylethylenyl)-
2-(thio)-pseudouracil
l-Methyl-3-(3-amino-3-carboxypropyl)
pseudouridine
l-Methyl-3-(3-amino-3-
carboxyproovl)pseudo-Uradine
l-Methyl-pseudo-UTP
2 (thio)pseudouracil
2′ deoxy uridine
2′ fluorouridine
2-(thio)uracil
2,4-(dithio)psuedouracil
2′-methyl, 2′-amino, 2′azido, 2′fluro-
guanosine
2′-Amino-2′-deoxy-uridine
2′-Azido-2′-deoxy-uridine
2′-Azido-deoxyuridine
2′-O-methylpseudouridine
2′ deoxyuridine
2′ fluorouridine
2′-Deoxy-2′-alpha-aminouridine TP
2′-Deoxy-2′-alpha-azidouridine TP
2-methylpseudouridine
3-(3 amino-3-carboxypropyl)uracil
4-(thio)pseudouracil
4-(thio)pseudouracil
5-(1,3-diazole-l-alkyl)uracil
5-(methoxy)uracil
5-(methoxycarbonylmethyl)-2-
(thio)uracil
5-(methoxycarbonyl-methyl)uracil
5-(methyl) 2(thio)uracil
5-(methyl) 2,4 (dithio)uracil
5-(methyl) 4 (thio)uracil
5-(methyl)-2-(thio)pseudouracil
5-(methyl)-2,4 (dithio)pseudouracil
5-(methyl)-4 (thio)pseudouracil
5-(methyl)pseudouracil
5-(methylaminomethyl)-2 (thio)uracil
5-(methylaminomethyl)-2,4 (dithio)uracil
5-(methylaminomethyl)-4-(thio)uracil
5-(propyny1)uracil
5-(trifluoromethyl)uracil
5-aminoallyl-uridine
5-bromo-uridine
5-iodo-uridine
5-uracil
6 (azo)uracil
6-(azo)uracil
6-aza-uridine
allyamino-uracil
aza uracil
deaza uracil
N3 (methyl)uracil
Pseudo-uridine-1-2-ethanoic acid
pseudouracil
4-Thio-pseudouridine
1-(2,4,6-Trimethyl-benzyl)pseudo-uridine
1-(2,4,6-Trimethyl-phenyl)pseudo-
uridine
1-(2-Amino-2-carboxyethyl)pseudo-
uridine
1-(2-Amino-ethyl)pseudouridine
1-(2-Hydroxyethyl)pseudouridine
1-(2-Methoxyethyl)pseudouridine
1-(3,4-Bis-
trifluoromethoxvbenzvl)pseudouridine
1-(3,4-Dimethoxybenzyl)pseudouridine
1-(3-Amino-3-carboxypropyl)pseudo-
uridine
1-(3-Amino-propyl)pseudouridine
1-(3-Cyclopropyl-prop-2-
ynyl)pseudouridine TP
1-(4-Amino-4-
carboxybutyl)pseudouridine
1-(4-Amino-benzyl)pseudouridine
1-(4-Amino-butyl)pseudouridine
1-(4-Amino-phenyl)pseudouridine
1-(4-Azidobenzyl)pseudouridine
1-(4-Bromobenzyl)pseudouridine
1-(4-Chlorobenzyl)pseudouridine
1-(4-Fluorobenzyl)pseudouridin
1-(4-Iodobenzyl)pseudouridine
1-(4-
Methanesulfonvlbenzvl)pseudouridine
1-(4-Methoxybenzyl)pseudouridine
1-(4-Methoxy-benzyl)pseudouridine
1-(4-Methoxy-phenyl)pseudouridine
1-(4-Methylbenzyl)pseudouridine
1-(4-Methyl-benzyl)pseudouridine
1-(4-Nitrobenzyl)pseudouridine
1-(4-Nitro-benzy!)pseudouridine
1-Cyclohexylmethyl-pseudo-uridine
1-Cyclohexyl-pseudo-uridine
1-Cyclooctylmethyl-pseudo-uridine
1-Cyclooctyl-pseudo-uridine
1-Cyclopentylmethyl-pseudo-uridine
1-Cyclopentyl-pseudo-uridine
1-Cyclopropylmethyl-pseudo-uridine
1-Cyclopropyl-pseudo-uridine
1-Ethyl-pseudo-uridine
1-Hexyl-pseudo-uridine
1-Homoallylpseudouridine
1-Hydroxymethylpseudouridine
1-iso-propyl-pseudo-uridine
1-Me-2-thio-pseudo-uridine
1-Me-4-thio-pseudo-uridine
1-Me-alpha-thio-pseudo-uridine
1-Methanesulfonylmethylpseudouridine
1-Methoxymethylpseudouridine uridine
l-Methyl-6-(2,2,2-Trifluoroethyl)pseudo-
uridine
l-Methyl-6-(4-morpholino)-pseudo-
uridine
1-Methyl-6-(4-thiomorpholino)-pseudo-
uridine
l-Methyl-6-(substituted phenyl)pseudo-
uridine
l-Methyl-6-amino-pseudo-uridine
l-Methyl-6-azido-pseudo-uridine
1-Methyl-6-bromo-pseudo-uridine
l-Methyl-6-butyl-pseudo-uridine
1-Methyl-6-chloro-pseudo-uridine
1-Methyl-6-cyano-pseudo-uridine
1-Methyl-6-dimethylamino-pseudo-
uridine
1-Vinylpseudouridine
2,2′-anhydro-uridine
2′-bromo-deoxyuridine
2′-F-5-Methyl-2′-deoxy-uridine
2′-OMe-5-Me-uridine
2′-OMe-pseudouridine
2′-alpha-Ethynyluridine
2′-alpha-Trifluoromethyluridine
2′-beta-Ethynyluridine
2′-beta-Trifluoromethyluridiner
2′-Deoxy-2′,2′-difluorouridine
2′-Deoxy-2′-a-mercaptouridin
2′-Deoxy-2′-alpha-thiomethoxyuridine
2′-Deoxy-2′-beta-aminouridine
2′-Deoxy-2′-beta-azidouridine
2′-Deoxy-2′-beta-bromouridine
2′-Deoxy-2′-beta-chlorouridine
2′-Deoxy-2′-beta-fluorouridine
2′-Deoxy-2′-beta-iodouridine
2′-Deoxy-2′-beta-mercaptouridine
2′-Deoxy-2′-beta-thiomethoxyuridine
2-methoxy-4-thio-uridine
2-methoxyuridine
2′-O-Methyl-5-(1-propynyl)uridine
3-Alkyl-pseudo-uridine
4′-Azidouridine
4′-Carbocyclic uridine
4′-Ethynyluridine
5-(1-Propynyl)ara-uridine
5-(2-Furanyl)uridine
5-Cyanouridine
6-Phenyl-pseudo-uridine
6-Propyl-pseudo-uridine
6-tert-Butyl-pseudo-uridine
6-Trifluoromethoxy-pseudo-uridine
6-Trifluoromethyl-pseudo-uridine
alpha-thio-pseudo-uridine
Pseudouridine 1-(4-
methylbenzenesulfonic
acid)
Pseudouridine 1-(4-methylbenzoic acid)
TP
Pseudouridine l-[3-(2-
ethoxy)]propionic acid
Pseudouridine l-[3-{2-(2-[2-(2-ethoxy)-
ethoxy]-ethoxy)-ethoxy}]propionic
acid
Pseudouridine l-[3-{2-(2-[2-{2(2-
ethoxy)-ethoxy}-ethoxy]-ethoxy)-
2-(aminoalkyl)adenine
2-(aminopropyl)adenine
2-(halo)adenine
2-(propyl)adenine
2′-azido-2′-deoxy-adenosine
2′-Deoxy-2′-alpha-aminoadenosine
2′-Deoxy-2′-alpha-azidoadenosine
6-(alkyl)adenine
6-(methyl)adenine
2-Azidoadenosine
2′-beta-Ethynyladenosine
2-Bromoadenosine
2′-beta-Trifluoromethyladenosine
2-Chloroadenosine
2′-Deoxy-2′,2′-difluoroadenosine
2′-Deoxy-2′-alpha-mercaptoadenosine
2′-Deoxy-2′-alpha-
thiomethoxyadenosine
2′-Deoxy-2′-beta-aminoadenosine
2′-Deoxy-2′-beta-azidoadenosine
2′-Deoxy-2′-beta-bromoadenosine
2′-Deoxy-2′-beta-chloroadenosine
2′-Deoxy-2′-beta-fluoroadenosine
2′-Deoxy-2′-beta-iodoadenosine
2′-Deoxy-2′-beta-mercaptoadenosine
2′-Deoxy-2′-beta-thiomethoxyadenosine
2-Fluoroadenosine
2-Iodoadenosine
2-Mercaptoadenosine
2-methoxy-adenine
2-methylthio-adenine
2-Trifluoromethyladenosine
3-Deaza-3-bromoadenosine
3-Deaza-3-chloroadenosine
3-Deaza-3-fluoroadenosine
3-Deaza-3-iodoadenosine
3-Deazaadenosine
4′-Azidoadenosine
4′-Carbocyclic adenosine
4′-Ethynyladenosine
5 (trifluoromethyl)cytosine
5-(alkyl)cytosine
5-(alkynyl)cytosine
5-(halo)cytosine
5-(propynyl)cytosine
5-(trifluoromethyl)cytosine
5-bromo-cytidine
5-iodo-cytidine
5-propynyl cytosine
6-(azo)cytosine
6-aza-cytidine
aza cytosine
deaza cytosine
N4 (acetyl)cytosine
l-methyl-1-deaza-pseudoisocytidine
1-methyl-pseudoisocytidine
2-methoxy-5-methyl-cytidine
2-methoxy-cytidine
2-thio-5-methyl-cytidine
4-methoxy-1-methyl-pseudoisocytidine
4-methoxy-pseudoisocytidine
4-thio-l-methyl-1-deaza-
pseudoisocytidine
4-thio-1-methyl-pseudoisocytidine
4-thio-pseudoisocytidine
5-aza-zebularine
5-methyl-zebularine
pyrrolo-pseudoisocytidine
zebularine
(E)-5-(2-Bromo-vinyl)cytidine
2,2′-anhydro-cytidine
5-Ethynylcytidine
5′-Homo-cytidine
5-Methoxycytidine
5-Trifluoromethyl-Cytidine
N4-Amino-cytidine
N4-Benzoyl-cytidine
pseudoisocytidine
6-thio-guanosine
7-deaza-guanosine
8-oxo-guanosine
Nl-methyl-guanosine
alpha-thio-guanosine
2-(propyl)guanine
2-(alkyl)guanine
2′-Amino-2′-deoxy-guanosine
2′-Azido-2′-deoxy-guanosine
2′-Deoxy-2′-alpha-aminoguanosine
2′-Deoxy-2′-alpha-azidoguanosine
6-(methyl)guanine
6-(alkyl)guanine
6-(methyl)guanine
6-methyl-guanosine
7-(alkyl)guanine
7-(deaza)guanine
7-(methyl)guanine
7-(alkyl)guanine
7-(deaza)guanine
7-(methyl)guanine
8-(alkyl)guanine
8-(alkynyl)guanine
8-(halo)guanine
2′-Deoxy-2′-alpha-
thiomethoxyguanosine
2′-Deoxy-2′-beta-aminoguanosine
2′-Deoxy-2′-beta-azidoguanosine
2′-Deoxy-2′-beta-bromoguanosine
2′-Deoxy-2′-beta-chloroguanosine
2′-Deoxy-2′-beta-fluoroguanosine
2′-Deoxy-2′-beta-iodoguanosine
2′-Deoxy-2′-beta-mercaptoguanosine
2′-Deoxy-2′-beta-thiomethoxyguanosine
4′-Azidoguanosine
4′-Carbocyclic guanosine
4′-Ethynylguanosine
5′-Homo-guanosine
8-bromo-guanosine
9-Deazaguanosine
N2-isobutyl-guanosine
7-methylinosine
allyamino-thymidine
aza thymidine
deaza thymidine
deoxy-thymidine
5-propynyl uracil
alpha-thio-uridine
1-(aminoalkylamino-carbonylethylenyl)-
2(thio)-pseudouracil
1-(aminoalkylaminocarbonylethylenyl)-
2,4-(dithio)pseudouracil
1-(aminoalkylaminocarbonylethylenyl)-4
(thio)pseudouracil
1-(aminoalkylaminocarbonylethylenyl)-
pseudouracil
1-(aminocarbonylethylenyl)-2(thio)-
pseudouracil
1-(aminocarbonylethylenyl)-2,4-
4-(thio)uracil
4-thiouracil
5-(l,3-diazole-1-alkyl)uracil
5-(2-aminopropyl)uracil
5-(aminoalkyl)uracil
5-(dimethylaminoalkyl)uracil
5-(guanidiniumalkyl)uracil
5-(methoxycarbonylmethyl)-2-
(thio)uracil
5-(methoxycarbonyl-methyl)uracil
5-(methyl)-2-(thio)uracil
5-(methyl)-2,4-(dithio)uracil
5 (methyl) 4 (thio)uracil
5 (methylaminomethyl)-2 (thio)uracil
5 (methylaminomethyl)-2,4 (dithio)uracil
5 (methylaminomethyl)-4 (thio)uracil
5 (propynyl)uracil
5 (trifluoromethyl)uracil
5-(2-aminopropyl)uracil
5-(alkyl)-2-(thio)pseudouracil
5-(alkyl)-2,4 (dithio)pseudouracil
5-(alkyl)-4 (thio)pseudouracil
5-(alkyl)pseudouracil
5-(alkyl)uracil
5-(alkynyl)uracil
5-(allylamino)uracil
5-(cyanoalkyl)uracil
5-(dialkylaminoalkyl)uracil
5-(dimethylaminoalkyl)uracil
5-(guanidiniumalkyl)uracil
5-(halo)uracil
1-carboxymethyl-pseudouridine
l-methyl-1-deaza-pseudouridine
1-propynyl-uridine
l-taurinomethyl-1-methyl-uridine
l-taurinomethyl-4-thio-uridine
1-taurinomethyl-pseudouridine
2-methoxy-4-thio-pseudouridine
2-thio-l-methyl-1-deaza-pseudouridine
2-thio-1-methyl-pseudouridine
2-thio-5-aza-uridine
2-thio-dihydropseudouridine
2-thio-dihydrouridine
2-thio-pseudouridine
4-methoxy-2-thio-pseudouridine
4-methoxy-pseudouridine
4-thio-1-methyl-pseudouridine
4-thio-pseudouridine
5-aza-uridine
dihydropseudouridine
(±)1-(2-Hydroxypropyl)pseudouridine
(2R)-l-(2-Hydroxypropyl)pseudouridine
(2S)-l-(2-Hydroxypropyl)pseudouridine
(E)-5-(2-Bromo-vinyl)ara-uridine
(E)-5-(2-Bromo-vinyl)uridine
(Z)-5-(2-Bromo-vinyl)ara-uridine
(Z)-5-(2-Bromo-vinyl)uridine
1-(2,2,2-Trifluoroethyl)-pseudouridine
1-(2,2,3,3,3-
Pentafluoropropyl)pseudouridine
1-(2,2-Diethoxyethyl)pseudouridine
1-(2,4,6-Trimethylbenzyl)pseudouridine
1(4-Nitro-phenyl)pseudouridine
1-(4-Thiomethoxybenzyl)pseudouridine
1-(4-
Trifluoromethoxybenzvl)pseudouridine
1-(4-
Trifluoromethylbenzyl)pseudouridine
1-(5-Amino-pentyl)pseudouridine
1-(6-Amino-hexyl)pseudouridine
1,6-Dimethyl-pseudouridine
l-[3-(2-{2-[2-(2-Aminoethoxy)-ethoxy]-
ethoxy}-ethoxy)-propionyl]pseudouridine
1-{3-[2-(2-Aminoethoxy)-ethoxy]-
propionvl} pseudouridine
1-Acetylpseudouridine
l-Alkyl-6-(1-propynyl)-pseudo-uridine
l-Alkyl-6-(2-propynyl)-pseudo-uridine
l-Alkyl-6-allyl-pseudo-uridine
l-Alkyl-6-ethynyl-pseudo-uridine
l-Alkyl-6-homoallyl-pseudo-uridine
l-Alkyl-6-vinyl-pseudo-uridine
1-Allylpseudouridine
1-Aminomethyl-pseudo-uridine
1-Benzoylpseudouridine
1-Benzyloxymethylpseudouridine
1-Benzyl-pseudo-uridine
l-Biotinyl-PEG2-pseudouridine
1-Biotinylpseudouridine
1-Butyl-pseudo-uridine
1-Cyanomethylpseudouridine
1-Cyclobutylmethyl-pseudo-uridine
1-Cyclobutyl-pseudo-uridine
1-Cycloheptylmethyl-pseudo-uridine
1-Cycloheptyl-pseudo-uridine
l-Methyl-6-ethoxy-pseudo-uridine
l-Methyl-6-ethylcarboxylate-pseudo-
uridine
l-Methyl-6-ethyl-pseudo-uridine
l-Methyl-6-fluoro-pseudo-uridine
l-Methyl-6-formyl-pseudo-uridine
1-Methyl-6-hydroxyamino-pseudo-
uridine
l-Methyl-6-hydroxy-pseudo-uridine
l-Methyl-6-iodo-pseudo-uridine
l-Methyl-6-iso-propyl-pseudo-uridine
l-Methyl-6-methoxy-pseudo-uridine
l-Methyl-6-methylamino-pseudo-uridine
l-Methyl-6-phenyl-pseudo-uridine
l-Methyl-6-propyl-pseudo-uridine
l-Methyl-6-tert-butyl-pseudo-uridine
1-Methyl-6-trifluoromethoxy-pseudo-
uridine
l-Methyl-6-trifluoromethyl-pseudo-
uridine
1-Morpholinomethylpseudouridine
1-Pentyl-pseudo-uridineuridine
1-Phenyl-pseudo-uridine
1-Pivaloylpseudouridine
1-Propargylpseudouridine
1-Propyl-pseudo-uridine
1-propynyl-pseudouridine
1-p-tolyl-pseudo-uridine
1-tert-Butyl-pseudo-uridine
1-Thiomethoxymethylpseudouridine
1-Thiomorpholinomethylpseudouridine
1-Trifluoroacetylpseudouridine
1-Trifluoromethyl-pseudouridine
5-Dimethylaminouridine
5′-Homo-uridine
5-iodo-2′-fluoro-deoxyuridine
5-Phenylethynyluridine
5-Trideuteromethyl-6-deuterouridine
5-Trifluoromethyl-Uridine
5-Vinylarauridine
6-(2,2,2-Trifluoroethyl)-pseudo-uridine
6-(4-Morpholino)-pseudo-uridine
6-(4-Thiomorpholino)-pseudo-uridine
6-(Substituted-Phenyl)-pseudo-uridine
6-Amino-pseudo-uridine
6-Azido-pseudo-uridine
6-Bromo-pseudo-uridine
6-Butyl-pseudo-uridine
6-Chloro-pseudo-uridine
6-Cyano-pseudo-uridine
6-Dimethylamino-pseudo-uridine
6-Ethoxy-pseudo-uridine
6-Ethylcarboxylate-pseudo-uridine
6-Ethyl-pseudo-uridine
6-Fluoro-pseudo-uridine
6-Formyl-pseudo-uridine
6-Hydroxyamino-pseudo-uridine
6-Hydroxy-pseudo-uridine
6-Iodo-pseudo-uridine
6-iso-Propyl-pseudo-uridine
6-Methoxy-pseudo-uridine
6-Methylamino-pseudo-uridine
6-Methyl-pseudo-uridine
6-Phenyl-pseudo-uridine
ethoxy}]propionic acid
Pseudouridine l-[3-{2-(2-[2-ethoxy]-
ethoxy)-ethoxv}]propionic acid
Pseudouridine l-[3-{2-(2-ethoxy)-
ethoxv}] propionic acid
Pseudouridine 1-methylphosphonic
acid
Pseudouridine TP 1-methylphosphonic
acid diethyl ester
Pseudo-uridine-N1-3-propionic acid
Pseudo-uridine-N1-4-butanoic acid
Pseudo-uridine-N1-5-pentanoic acid
Pseudo-uridine-N1-6-hexanoic acid
Pseudo-uridine-Nl-7-heptanoic acid
Pseudo-uridine-N1-methy1-p-benzoic
acid
Pseudo-uridine-N1-p-benzoic acid

In an embodiment, a TREM, a TREM core fragment or a TREM fragment described herein comprises a modification provided in Table 6, or a combination thereof. The modifications provided in Table 6 occur naturally in RNAs, and are used herein on a synthetic TREM, a TREM core fragment or a TREM fragment at a position that does not occur in nature.

TABLE 6
Additional exemplary modifications

Modification
2-methylthio-N6-(cis-
hvdroxvisopentenvl) adenosine
2-methylthio-N6-methyladenosine
2-methylthio-N6-
threonyl carbamoyladenosine
N6-glycinylcarbamoyladenosine
N6-isopentenyladenosine
N6-methyladenosine
N6-threonylcarbamoyladenosine
2′-O-ribosyladenosine (phosphate)
isopenteny ladenosine
N6-(cis-hydroxyisopentenyl)adenosine
N6,2′-O-dimethyladenosine
N6,2′-O-dimethyladenosine
N6,N6,2′-O-trimethyladenosine
N6,N6-dimethyladenosine
N6-acetyladenosine
N6-hydroxynorvalylcarbamoyladenosine
N6-methyl-N6-
threonylcarbamoyladenosine
2-methyladenosine
2-methylthio-N⁶-isopentenyladenosine
2-thiocytidine
3-methylcytidine
5-formylcytidine
5-hydroxymethylcytidine
5-methylcytidine
N4-acetylcytidine
2′-O-methylcytidine
2′-O-methylcytidine
5,2′-O-dimethylcytidine
5-formyl-2′-O-methylcytidine
lysidine
N4,2′-O-dimethy lcytidine
N4-acetyl-2′-O-methylcytidine
N4-methylcytidine
N4,N4-Dimethyl-2′-OMe-Cytidine
7-methylguanosine
N2,2′-O-dimethylguanosine
N2-methylguanosine
wyosme
1,2′-O-dimethylguanosine
1-methylguanosine
(3-(3-amino-3-carboxypropyl)uridine
l-methyl-3-(3-amino-5-
carboxypropyl)pseudouridine
1-methylpseduouridine
1-methyl-pseudouridine
2′-O-methyluridine
2′-O-methylpseudouridine
2′-O-methyluridine
2-thio-2′-O-methyluridine
3-(3-amino-3-carboxypropyl)uridine
3,2′-0-dimethyluridine
3-Methyl-pseudo-Uridine
4-thiouridine
5-(carboxyhydroxymethyl)uridine
5-(carboxyhydroxymethyl)uridine
methyl ester
5,2′-O-dimethyluridine
5,6-dihydro-uridine
5-aminomethyl-2-thiouridine
5-carbamoylmethyl-2′-0-methyluridine
5-carbamoylmethyluridine
5-carboxyhydroxymethyluridine
5-carboxyhydroxymethyluridine
methyl ester
5-carboxymethylaminomethyl-2′-O-
methyluridine
5-carboxymethylaminomethyl-2-
thiouridine
5-carboxymethylaminomethyl-2-
thiouridine
5-carboxymethylaminomethyluridine
1,2′-O-dimethyladenosine
1-methyladenosine
2′-O-methyladenosine
2′-O-ribosyladenosine (phosphate)
2-methyladenosine
2-methylthio-N6 isopentenyladenosine
2-methylthio-N6-
hydroxynorvalyl carbamoyladenosine
2′-O-methyladenosine
2′-O-methylguanosine
2′-O-ribosylguanosine (phosphate)
2′-O-methylguanosine
2′-O-ribosylguanosine (phosphate)
7-aminomethyl-7-deazaguanosine
7-cyano-7-deazaguanosine
archaeosine
methylwyosine
N2,7-dimethylguanosine
N2,N2,2′-O-trimethylguanosine
N2,N2,7-trimethylguanosine
N2,N2-dimethylguanosine
N2,7,2′-O-trimethylguanosine
1-methylinosine
mosme
1,2′-O-dimethylinosine
2′-O-methylinosine
2′-O-methylinosine
epoxyqueuosine
galactosyl-queuosine
mannosyl-queuosine
2′-O-methyluridine
2-thiouridine
3-methyluridine
5-carboxymethyluridine
5-hydroxyuridine
5-methyluridine
5-taurinomethyl-2-thiouridine
5-taurinomethyluridine
dihydrouridine
pseudouridine
5-carboxymethylaminomethyluridine
5-Carbamoylmethyluridine
5-methoxycarbonylmethyl-2′-O-
methyluridine
5-methoxycarbonylmethy 1-2-thiouridine
5-methoxycarbonylmethyluridine
5-methoxyuridine
5-methyl-2-thiouridine
5-methylaminomethyl-2-selenouridine
5-methylaminomethyl-2-thiouridine
5-methylaminomethyluridine
5-Methyldihydrouridine
5-Oxyacetic acid-Uridine
5-Oxyacetic acid-methyl ester-Uridin
Nl-methyl-pseudo-uridine
uridine 5-oxyacetic acid
uridine 5-oxyacetic acid methyl ester
3-(3-Amino-3-carboxypropyl)-Uridine
5-(iso-Pentenylaminomethyl)-2-
thiouridine
5-(iso-Pentenylaminomethyl)-2′-O-
methyluridine
5-(iso-Pentenylaminomethyl)uridine
wybutosine
hydroxywybutosine
isowyosme
peroxywybutosine
undermodified hydroxywybutosine
4-demethylwyosine
altriol

In an embodiment, a TREM, a TREM core fragment or a TREM fragment described herein comprises a non-naturally occurring modification provided in Table 7, or a combination thereof.

TABLE 7
Additional exemplary non-naturally occurring modifications

Modification
2,6-(diamino)purine
1-(aza)-2-(thio)-3-(aza)-phenoxazin-1-yl
1,3-( diaza)-2-( oxo )-phenthiazin-1-yl
1,3-(diaza)-2-(oxo)-phenoxazin-1-yl
1,3,5-(triaza)-2,6-(dioxa)-naphthalene
2 (amino)purine
2,4,5-(trimethyl)phenyl
2′ methyl, 2′amino, 2′azido,
2′fluro-cytidine
2′ methyl, 2′amino, 2′azido,
2′fluro-adenine
2′methyl, 2′amino, 2′azido,
2′fluro-uridine
2′-amino-2′-deoxyribose
2-amino-6-Chloro-purine
2-aza-inosinyl
2′-azido-2′-deoxyribose
2′fluoro-2′-deoxyribose
2′-fluoro-modified bases
2′-O-methyl-ribose
2-oxo-7-aminopyridopyrimidin-3-yl
2-oxo-pyridopyrimidine-3-yl
2-pyridinone
3 nitropyrrole
3-(methyl)-7-(propynyl)isocarbostyrilyl
3-(methyl)isocarbostyrilyl
4-(fluoro)-6-(methyl)benzimidazole
4-(methyl)benzimidazole
4-(methyl)indolyl
4,6-(dimethyl)indolyl
5 nitroindole
5 substituted pyrimidines
5-(methyl)isocarbostyrilyl
5-nitroindole
6-(aza)pyrimidine
9-(methyl)-imidizopyridinyl
aminoindolyl
anthracenyl
bis-ortho-(aminoalkylhydroxy)-6-
phenyl-pyrrolo-nvrimidin-2-on-3-yl
bis-ortho-substituted-6-phenyl-pyrrolo-
pyrimidin-2-on-3-yl
difluorotolyl
hypoxanthine
imidizopyridinyl
inosinyl
isocarbostyrilyl
isoguanosine
N2-substituted purines
N6-methyl-2-amino-purine
N6-substituted purines
N-alkylated derivative
napthalenyl
nitrobenzimidazolyl
nitroimidazolyl
nitroindazolyl
nitropyrazolyl
nubularine
O6-substituted purines
O-alkylated derivative
ortho-(aminoalkylhydroxy)-6-phenyl-
pyrrolo-pyrimidin-2-on-3-yl
ortho-substituted-6-phenyl-pyrrolo-
pyrimidin-2-on-3-yl
Oxoformycin TP
para-(aminoalkylhydroxy)-6-phenyl-
pyrrolo-pyrimidin-2-on-3-yl
para-substituted-6-phenyl-pyrrolo-
6-(azo)thymine
6-(methyl)-7-(aza)indolyl
6-chloro-purine
6-phenyl-pyrrolo-pyrimidin-2-on-3-yl
7-(aminoalkylhydroxy)-l-(aza)-2-(thio)-
3-(aza)-phenthiazin-1-yl
7-(aminoalkylhydroxy)-1-(aza)-2-(thio)-
3-(aza)-phenoxazin-1-yl
7-(aminoalkylhydroxy)-1,3-(diaza)-2-
(oxo)-phenoxazin-1-yl
7-(aminoalkylhydroxy)-1,3-(diaza)-2-
( oxo )-phenthiazin-1-yl
7-(aminoalkylhydroxy)-1,3-(diaza)-2-
(oxo)-phenoxazin-1-yl
7-(aza)indolyl
7-(guanidiniumalkylhydroxy)-1-(aza)-2-
(thio )-3-(aza)-phenoxazinl-yl
7-(guanidiniumalkylhydroxy)-1-(aza)-2-
(thio )-3-(aza)-phenthiazin-1-yl
7-(guanidiniumalkylhydroxy)-1-(aza)-2-
(thio)-3-(aza)-phenoxazin-1-yl
7-(guanidiniumalkylhydroxy)-1,3-
(diaza)-2-(oxo)-phenoxazin-1-yl
7-(guanidiniumalkyl-hydroxy)-1,3-
(diaza)-2-( oxo )-phenthiazin-1-yl
7-(guanidiniumalkylhydroxy)-1,3-
(diaza)-2-( oxo )-phenoxazin-1-yl
7-(propynyl)isocarbostyrilyl
7-(propynyl)isocarbostyrilyl,
propynyl-7-(aza)indolyl
7-deaza-inosinyl
7-substituted 1-(aza)-2-(thio)-3-
(aza)-phenoxazin-1-yl
7-substituted 1,3-(diaza)-2-
(oxo)-phenoxazin-1-yl
pyrimidin-2-on-3-yl
pentacenyl
phenanthracenyl
phenyl
propynyl-7-(aza)indolyl
pyrenyl
pyridopyrimidin-3-yl
pyridopyrimidin-3-yl, 2-oxo-
7-amino-pyridopyrimidin-3-yl
pyrrolo-pyrimidin-2-on-3-yl
pyrrolopyrimidinyl
pyrrolopyrizinyl
stilbenzyl
substituted 1,2,4-triazoles
tetracenyl
tubercidine
xanthine
Xanthosine
2-thio-zebularine
5-aza-2-thio-zebularine
7-deaza-2-amino-purine
pyridin-4-one ribonucleoside
2-Amino-riboside
Formycin A
Formycin B
Pyrrolosine
2′-OH-ara-adenosine
2′-OH-ara-cytidine
2′-OH-ara-uridine
2′-OH-ara-guanosine
5-(2-carbomethoxyvinyl)uridine
N6-(19-Amino-
pentaoxanonadecyl)adenosine

In an embodiment, a TREM, a TREM core fragment or a TREM fragment described herein comprises a non-naturally occurring modification provided in Table 8, or a combination thereof.

TABLE 8
Exemplary backbone modifications

Modification
3′-alkylene phosphonates
3′-amino phosphoramidate
alkene containing backbones
aminoalkylphosphoramidates
aminoalkylphosphotriesters
boranophosphates
—CH2—O—N(CH3)—CH2—
—CH2—N(CH3)—N(CH3)—CH2—
—CH2—NH—CH2—
chiral phosphonates
chiral phosphorothioates
formacetyl and thioformacetyl
backbones
methylene (methylimino)
methylene formacetyl and
thioformacetyl backbones
methyleneimino and
methylenehydrazino backbones
morpholino linkages
—N(CH3)—CH2—CH2—
oligonucleosides with heteroatom
intenucleoside linkage
phosphinates
phosphoramidates
phosphorodithioates
phosphorothioate
intenucleoside linkages
phosphorothioates
phosphotriesters
Peptide nucleic acid (PNA)
siloxane backbones
sulfamate backbones
Sulfide, sulfoxide, and
sulfone backbones
sulfonate and sulfonamide backbones
thionoalkylphosphonates
thionoalkylphosphotriesters
thionophosphoramidates
(S) 5′-C-methyl with phosphate
(R) 5′-C-methyl with phosphate
DNA
(R) 5′-C-methyl
methylphosphonates
phosphonoacetates
Phosphorothioate
Constrained nucleic acid (CNA)
2′-O-methyl
2′-O-methoxyethyl (MOE)
2′ Fluoro
Locked nucleic acid (LNA)
(S)-constrained ethyl (cEt)
Fluoro hexitol nucleic acid (FHNA)
5′-phosphorothioate
Phosphorodiamidate
Morpholino Oligomer (PMO)
Tricyclo-DNA (tcDNA)
(S) 5′-C-methyl
(E)-vinylphosphonate
Methyl phosphonate
(S) 5′-C-methyl with phosphate
(R) 5′-C-methyl with phosphate
DNA
(R) 5′-C-methyl
GNA (glycol nucleic acid)
alkyl phosphonates
Phosphorothioate
Constrained nucleic acid (CNA)
2′-O-methyl
2′-O-methoxyethyl (MOE)
2′ Fluoro
Locked nucleic acid (LNA)
(S)-constrained ethyl (cEt)
Fluoro hexitol nucleic acid (FHNA)
5′-phosphorothioate
Phosphorodiamidate
Morpholino Oligomer (PMO)
Tricyclo-DNA (tcDNA)
(S) 5′-C-methyl
(E)-vinylphosphonate
Methyl phosphonate
GNA (glycol nucleic acid)
alkyl phosphonates

In an embodiment, a TREM, a TREM core fragment or a TREM fragment described herein comprises a non-naturally occurring modification provided in Table 9, or a combination thereof.

TABLE 9
Exemplary non-naturally occurring backbone modificiations

Name of synthetic backbone modifications
Phosphorothioate
Constrained nucleic acid (CNA)
2′ O′methylation
2′-O-methoxyethylribose (MOE)
2′ Fluoro
Locked nucleic acid (LNA)
(S)-constrained ethyl (cEt)
Fluoro hexitol nucleic acid (FHNA)
5′phosphorothioate
Phosphorodiamidate
Morpholino Oligomer (PMO)
Tricyclo-DNA (tcDNA)
(S) 5′-C-methyl
(E) -vinylphosphonate
Methyl phosphonate
(S) 5′-C-methyl with phosphate

### TREM, TREM Core Fragment and TREM Fragment Fusions

In an embodiment, a TREM, a TREM core fragment or a TREM fragment disclosed herein comprises an additional moiety, e.g., a fusion moiety. In an embodiment, the fusion moiety can be used for purification, to alter folding of the TREM, TREM core fragment or TREM fragment, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM, TREM core fragment or TREM fragment. In an embodiment, the fusion moiety can be encoded by the same or different nucleic acid molecule that encodes the TREM, TREM core fragment or TREM fragment.

### TREM Consensus Sequence

In an embodiment, a TREM disclosed herein comprises a consensus sequence provided herein.

In an embodiment, a TREM disclosed herein comprises a consensus sequence of Formula I_{ZZZ}, wherein ZZZ indicates any of the twenty amino acids and Formula I corresponds to all species.

In an embodiment, a TREM disclosed herein comprises a consensus sequence of Formula II_{ZZZ}, wherein ZZZ indicates any of the twenty amino acids and Formula II corresponds to mammals.

In an embodiment, a TREM disclosed herein comprises a consensus sequence of Formula III_{ZZZ}, wherein ZZZ indicates any of the twenty amino acids and Formula III corresponds to humans.

In an embodiment, ZZZ indicates any of the twenty amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, a TREM disclosed herein comprises a property selected from the following:

a) under physiological conditions residue R₀ forms a linker region, e.g., a Linker 1 region;

b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;

c) under physiological conditions residues R₈-R₉ forms a linker region, e.g., a Linker 2 region;

d) under physiological conditions residues —R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;

e) under physiological conditions residue —R₂₉ forms a linker region, e.g., a Linker 3 Region;

f) under physiological conditions residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;

g) under physiological conditions residue —[R₄₇]ₓ comprises a variable region, e.g., as described herein;

h) under physiological conditions residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or

i) under physiological conditions residue R₇₂ forms a linker region, e.g., a Linker 4 region.

### Alanine TREM Consensus Sequence

In an embodiment, a TREM disclosed herein comprises the sequence of Formula I_{ALA} (SEQ ID NO: 562),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀=absent;
R₁₄, R₅₇=are independently A or absent;
R₂₆=A, C, G or absent;
R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent;
R₁₁, R₃₅, R₆₅=are independently A, C, U or absent;
R₁, R₉, R₂₀, R₃₈, R₄₀, R₅₁, R₅₂, R₅₆=are independently A, G or absent;
R₇, R₂₂, R₂₅, R₂₇, R₂₉, R₄₆, R₅₃, R₇₂=are independently A, G, U or absent;
R₂₄, R₆₉=are independently A, U or absent;
R₇₀, R₇₁=are independently C or absent;
R₃, R₄=are independently C, G or absent;
R₁₂, R₃₃, R₃₆, R₆₂, R₆₈=are independently C, G, U or absent;
R₁₃, R₁₇, R₂₈, R₃₉, R₅₅, R₆₀, R₆₁=are independently C, U or absent;
R₁₀, R₁₉, R₂₃=are independently G or absent;
R₂=G, U or absent;
R₈, R₁₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA} (SEQ ID NO: 564),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I_{ARG} (SEQ ID NO: 565),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₅₇=A or absent;
R₉, R₂₇=are independently A, C, G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅, R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or absent;
R₁₃, R₁₇, R₄₁=are independently A, C, U or absent;
R₁₉, R₂₀, R₂₄, R₄₀, R₅₆=are independently A, G or absent;
R₁₄, R₁₅, R₇₂=are independently A, G, U or absent;
R₁₈=A, U or absent;
R₃₈=C or absent;
R₃₅, R₄₃, R₆₁=are independently C, G, U or absent;
R₂₈, R₅₅, R₅₉, R₆₀=are independently C, U or absent;
R₀, R₁₀, R₅₂=are independently G or absent;
R₈, R₃₉=are independently G, U or absent;
R₃₆, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ARG} (SEQ ID NO: 566),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=absent;
R₂₄, R₅₇=are independently A or absent;
R₄₁=A, C or absent;
R₃, R₇, R₃₄, R₅₀=are independently A, C, G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A, C, U or absent;
R₁, R₁₅, R₁₉, R₂₅, R₂₇, R₄₀, R₄₅, R₄₆, R₅₆, R₇₂=are independently A, G or absent;
R₁₄, R₂₉, R₆₃=are independently A, G, U or absent;
R₁₆, R₂₁=are independently A, U or absent;
R₃₈, R₆₁=are independently C or absent;
R₃₃, R₄₈=are independently C, G or absent;
R₄, R₉, R₁₁, R₄₃, R₆₂, R₆₄, R₆₉=are independently C, G, U or absent;
R₁₃, R₂₂, R₂₈, R₃₀, R₃₁, R₃₅, R₅₅, R₆₀, R₆₅=are independently C, U or absent;
R₀, R₁₀, R₂₀, R₂₃, R₅₁, R₅₂=are independently G or absent;
R₈, R₃₉, R₄₂=are independently G, U or absent;
R₁₇, R₃₆, R₅₃, R₅₄, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ARG} (SEQ ID NO: 567),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:

R₁₈=is absent;
R₁₅, R₂₁, R₂₄, R₄₁, R₅₇=are independently A or absent;
R₃₄, R₄₄=are independently A, C or absent;
R₃, R₅, R₅₈=are independently A, C, G or absent;
R₂, R₆, R₆₆, R₇₀=are independently N or absent;
R₃₇, R₄₉=are independently A, C, U or absent;
R₁, R₂₅, R₂₉, R₄₀, R₄₅, R₄₆, R₅₀=are independently A, G or absent;
R₁₄, R₆₃, R₆₈=are independently A, G, U or absent;
R₁₆=A, U or absent;
R₃₈, R₆₁=are independently C or absent;
R₇, R₁₁, R₁₂, R₂₆, R₄₈=are independently C, G or absent;
R₆₄, R₆₇, R₆₉=are independently C, G, U or absent;
R₄, R₁₃, R₂₂, R₂₈, R₃₀, R₃₁, R₃₅, R₄₃, R₅₅, R₆₀, R₆₂, R₆₅, R₇₁=are independently C, U or absent;
R₀, R₁₀, R₁₉, R₂₀, R₂₃, R₂₇, R₃₃, R₅₁, R₅₂, R₅₆, R₇₂=are independently G or absent;
R₈, R₉, R₃₂, R₃₉, R₄₂=are independently G, U or absent;
R₁₇, R₃₆, R₅₃, R₅₄, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I_{ASN} (SEQ ID NO: 568),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent;
R₄₁=A or absent;
R₁₄, R₄₈, R₅₆=are independently A, C, G or absent;
R₂, R₄, R₅, R₆, R₁₂, R₁₇, R₂₆, R₂₉, R₃₀, R₃₁, R₄₄, R₄₅, R₄₆, R₄₉, R₅₀, R₅₅, R₆₂, R₆₃, R₆₅, R₆₆, R₆₇, R₆₈, R₇₀, R₇₁=are independently N or absent;
R₁₁, R₁₃, R₂₂, R₄₂, R₅₅, R₅₉=are independently A, C, U or absent;
R₉, R₁₅, R₂₄, R₂₇, R₃₄, R₃₇, R₅₁, R₇₂=are independently A, G or absent;
R₁, R₇, R₂₅, R₆₉=are independently A, G, U or absent;
R₄₀, R₅₇=are independently A, U or absent;
R₆₀=C or absent;
R₃₃=C, G or absent;
R₂₁, R₃₂, R₄₃, R₆₄=are independently C, G, U or absent;
R₃, R₁₆, R₂₈, R₃₅, R₃₆, R₆₁=are independently C, U or absent;
R₁₀, R₁₉, R₂₀, R₅₂=are independently G or absent;
R₅₄=G, U or absent;
R₈, R₂₃, R₃₈, R₃₉, R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASN} (SEQ ID NO: 569),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent
R₂₄, R₄₁, R₄₆, R₆₂=are independently A or absent;
R₅₉=A, C or absent;
R₁₄, R₅₆, R₆₆=are independently A, C, G or absent;
R₁₇, R₂₉=are independently N or absent;
R₁₁, R₂₆, R₄₂, R₅₅=are independently A, C, U or absent;
R₁, R₉, R₁₂, R₁₅, R₂₅, R₃₄, R₃₇, R₄₈, R₅₁, R₆₇, R₆₈, R₆₉, R₇₀, R₇₂=are independently A, G or absent;
R₄₄, R₄₅, R₅₈=are independently A, G, U or absent;
R₄₀, R₅₇=are independently A, U or absent;
R₅, R₂₈, R₆₀=are independently C or absent;
R₃₃, R₆₅=are independently C, G or absent;
R₂₁, R₄₃, R₇₁=are independently C, G, U or absent;
R₃, R₆, R₁₃, R₂₂, R₃₂, R₃₅, R₃₆, R₆₁, R₆₃, R₆₄=are independently C, U or absent;
R₇, R₁₀, R₁₉, R₂₀, R₂₇, R₄₉, R₅₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ASN} (SEQ ID NO: 570),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent
R₂₄, R₄₀, R₄₁, R₄₆, R₆₂=are independently A or absent;
R₅₉=A, C or absent;
R₁₄, R₅₆, R₆₆=are independently A, C, G or absent;
R₁₁, R₂₆, R₄₂, R₅₅=are independently A, C, U or absent;
R₁, R₉, R₁₂, R₁₅, R₃₄, R₃₇, R₄₈, R₅₁, R₆₇, R₆₈, R₆₉, R₇₀=are independently A, G or absent;
R₄₄, R₄₅, R₅₈=are independently A, G, U or absent;
R₅₇=A, U or absent;
R₅, R₂₈, R₆₀=are independently C or absent;
R₃₃, R₆₅=are independently C, G or absent;
R₁₇, R₂₁, R₂₉=are independently C, G, U or absent;
R₃, R₆, R₁₃, R₂₂, R₃₂, R₃₅, R₃₆, R₄₃, R₆₁, R₆₃, R₆₄, R₇₁=are independently C, U or absent;
R₇, R₁₀, R₁₉, R₂₀, R₂₅, R₂₇, R₄₉, R₅₂, R₇₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I ASP (SEQ ID NO: 571),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀=absent
R₂₄, R₇₁=are independently A, C or absent;
R₃₃, R₄₆=are independently A, C, G or absent;
R₂, R₃, R₄, R₅, R₆, R₁₂, R₁₆, R₂₂, R₂₆, R₂₉, R₃₁, R₃₂, R₄₄, R₄₈, R₄₉, R₅₈, R₆₃, R₆₄, R₆₆, R₆₇, R₆₈, R₆₉=are independently N or absent;
R₁₃, R₂₁, R₃₄, R₄₁, R₅₇, R₆₅=are independently A, C, U or absent;
R₉, R₁₀, R₁₄, R₁₅, R₂₀, R₂₇, R₃₇, R₄₀, R₅₁, R₅₆, R₇₂=are independently A, G or absent;
R₇, R₂₅, R₄₂=are independently A, G, U or absent;
R₃₉=C or absent;
R₅₀, R₆₂=are independently C, G or absent;
R₃₀, R₄₃, R₄₅, R₅₅, R₇₀=are independently C, G, U or absent;
R₈, R₁₁, R₁₇, R₁₈, R₂₈, R₃₅, R₅₃, R₅₉, R₆₀, R₆₁=are independently C, U or absent;
R₁₉, R₅₂=are independently G or absent;
R₁=G, U or absent;
R₂₃, R₃₆, R₃₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ASP (SEQ ID NO: 572),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are independently absent;
R₉, R₄₀=are independently A or absent;
R₂₄, R₇₁=are independently A, C or absent;
R₆₇, R₆₈=are independently A, C, G or absent;
R₂, R₆, R₆₆=are independently N or absent;
R₅₇, R₆₃=are independently A, C, U or absent;
R₁₀, R₁₄, R₂₇, R₃₃, R₃₇, R₄₄, R₄₆, R₅₁, R₅₆, R₆₄, R₇₂=are independently A, G or absent;
R₇, R₁₂, R₂₆, R₆₅=are independently A, U or absent;
R₃₉, R₆₁, R₆₂=are independently C or absent;
R₃, R₃₁, R₄₅, R₇₀=are independently C, G or absent;
R₄, R₅, R₂₉, R₄₃, R₅₅=are independently C, G, U or absent;
R₈, R₁₁, R₁₃, R₃₀, R₃₂, R₃₄, R₃₅, R₄₁, R₄₈, R₅₃, R₅₉, R₆₀=are independently C, U or absent;
R₁₅, R₁₉, R₂₀, R₂₅, R₄₂, R₅₀, R₅₂=are independently G or absent;
R₁, R₂₂, R₄₉, R₅₈, R₆₉=are independently G, U or absent;
R₁₆, R₂₁, R₂₈, R₃₆, R₃₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASP (SEQ ID NO: 573),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are absent
R₉, R₁₂, R₄₀, R₆₅, R₇₁=are independently A or absent;
R₂, R₂₄, R₅₇=are independently A, C or absent;
R₆, R₁₄, R₂₇, R₄₆, R₅₁, R₅₆, R₆₄, R₆₇, R₆₈=are independently A, G or absent;
R₃, R₃₁, R₃₅, R₃₉, R₆₁, R₆₂=are independently C or absent;
R₆₆=C, G or absent;
R₅, R₈, R₂₉, R₃₀, R₃₂, R₃₄, R₄₁, R₄₃, R₄₈, R₅₅, R₅₉, R₆₀, R₆₃=are independently C, U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂=are independently G or absent;
R₂₂, R₅₈=are independently G, U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I_{CYS} (SEQ ID NO: 574),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀=absent
R₁₄, R₃₉, R₅₇=are independently A or absent;
R₄₁=A, C or absent;
R₁₀, R₁₅, R₂₇, R₃₃, R₆₂=are independently A, C, G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₈, R₄₉, R₅₅, R₆₃, R₆₄, R₆₆, R₆₇, R₆₈, R₆₉, R₇₀=are independently N or absent;
R₆₅=A, C, U or absent;
R₉, R₂₅, R₃₇, R₄₀, R₅₂, R₅₆=are independently A, G or absent;
R₇, R₂₀, R₅₁=are independently A, G, U or absent;
R₁₈, R₃₈, R₅₅=are independently C or absent;
R₂=C, G or absent;
R₂₁, R₂₈, R₄₃, R₅₀=are independently C, G, U or absent;
R₁₁, R₂₂, R₂₃, R₃₅, R₃₆, R₅₉, R₆₀, R₆₁, R₇₁, R₇₂=are independently C, U or absent;
R₁, R₁₉=are independently G or absent;
R₁₇=G, U or absent;
R₈, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{CYS} (SEQ ID NO: 575),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀, R₁₈, R₂₃=are absent;
R₁₄, R₂₄, R₂₆, R₂₉, R₃₉, R₄₁, R₄₅, R₅₇=are independently A or absent;
R₄₄=A, C or absent;
R₂₇, R₆₂=are independently A, C, G or absent;
R₁₆=A, C, G, U or absent;
R₃₀, R₇₀=are independently A, C, U or absent;
R₅, R₇, R₉, R₂₅, R₃₄, R₃₇, R₄₀, R₄₆, R₅₂, R₅₆, R₅₈, R₆₆=are independently A, G or absent;
R₂₀, R₅₁=are independently A, G, U or absent;
R₃₅, R₃₈, R₄₃, R₅₅, R₆₉=are independently C or absent;
R₂, R₄, R₁₅=are independently C, G or absent;
R₁₃=C, G, U or absent;
R₆, R₁₁, R₂₈, R₃₆, R₄₈, R₄₉, R₅₀, R₆₀, R₆₁, R₆₇, R₆₈, R₇₁, R₇₂=are independently C, U or absent;
R₁, R₃, R₁₀, R₁₉, R₃₃, R₆₃=are independently G or absent;
R₈, R₁₇, R₂₁, R₆₄=are independently G, U or absent;
R₁₂, R₂₂, R₃₁, R₃₂, R₄₂, R₅₃, R₅₄, R₆₅=are independently U or absent;
R₅₉=U, or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{CYS} (SEQ ID NO: 576),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀, R₁₈, R₂₃=are absent
R₁₄, R₂₄, R₂₆, R₂₉, R₃₄, R₃₉, R₄₁, R₄₅, R₅₇, R₅₈=are independently A or absent;
R₄₄, R₇₀=are independently A, C or absent;
R₆₂=A, C, G or absent;
R₁₆=N or absent;
R₅, R₇, R₉, R₂₀, R₄₀, R₄₆, R₅₁, R₅₂, R₅₆, R₆₆=are independently A, G or absent;
R₂₈, R₃₅, R₃₈, R₄₃, R₅₅, R₆₇, R₆₉=are independently C or absent;
R₄, R₁₅=are independently C, G or absent;
R₆, R₁₁, R₁₃, R₃₀, R₄₈, R₄₉, R₅₀, R₆₀, R₆₁, R₆₈, R₇₁, R₇₂=are independently C, U or absent;
R₁, R₂, R₃, R₁₀, R₁₉, R₂₅, R₂₇, R₃₃, R₃₇, R₆₃=are independently G or absent;
R₈, R₂₁, R₆₄=are independently G, U or absent;
R₁₂, R₁₇, R₂₂, R₃₁, R₃₂, R₃₆, R₄₂, R₅₃, R₅₄, R₅₉, R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I_{GLN} (SEQ ID NO: 577),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈=are absent;
R₁₄, R₂₄, R₅₇=are independently A or absent;
R₉, R₂₆, R₂₇, R₃₃, R₅₆=are independently A, C, G or absent;
R₂, R₄, R₅, R₆, R₁₂, R₁₃, R₁₆, R₂₁, R₂₂, R₂₅, R₂₉, R₃₀, R₃₁, R₃₂, R₃₄, R₄₁, R₄₂, R₄₄, R₄₅, R₄₆, R₄₈, R₄₉, R₅₀, R₅₈, R₆₂, R₆₃, R₆₆, R₆₇, R₆₈, R₆₉, R₇₀=are independently N or absent;
R₁₇, R₂₃, R₄₃, R₆₅, R₇₁=are independently A, C, U or absent;
R₁₅, R₄₀, R₅₁, R₅₂=are independently A, G or absent;
R₁, R₇, R₇₂=are independently A, G, U or absent;
R₃, R₁₁, R₃₇, R₆₀, R₆₄=are independently C, G, U or absent;
R₂₈, R₃₅, R₅₄, R₅₉, R₆₁=are independently C, U or absent;
R₁₀, R₁₉, R₂₀=are independently G or absent;
R₃₉=G, U or absent;
R₈, R₃₆, R₃₈, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLN} (SEQ ID NO: 578),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈, R₂₃=are absent
R₁₄, R₂₄, R₅₇=are independently A or absent;
R₁₇, R₇₁=are independently A, C or absent;
R₂₅, R₂₆, R₃₃, R₄₄, R₄₆, R₅₆, R₆₉=are independently A, C, G or absent;
R₄, R₅, R₁₂, R₂₂, R₂₉, R₃₀, R₄₈, R₄₉, R₆₃, R₆₇, R₆₈=are independently N or absent;
R₃₁, R₄₃, R₆₂, R₆₅, R₇₀=are independently A, C, U or absent;
R₁₅, R₂₇, R₃₄, R₄₀, R₄₁, R₅₁, R₅₂=are independently A, G or absent;
R₂, R₇, R₂₁, R₄₅, R₅₀, R₅₈, R₆₆, R₇₂=are independently A, G, U or absent;
R₃, R₁₃, R₃₂, R₃₇, R₄₂, R₆₀, R₆₄=are independently C, G, U or absent;
R₆, R₁₁, R₂₈, R₃₅, R₅₅, R₅₉, R₆₁=are independently C, U or absent;
R₉, R₁₀, R₁₉, R₂₀=are independently G or absent;
R₁, R₁₆, R₃₉=are independently G, U or absent;
R₈, R₃₆, R₃₈, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLN} (SEQ ID NO: 579),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈, R₂₃=are absent
R₁₄, R₂₄, R₄₁, R₅₇=are independently A or absent;
R₁₇, R₇₁=are independently A, C or absent;
R₅, R₂₅, R₂₆, R₄₆, R₅₆, R₆₉=are independently A, C, G or absent;
R₄, R₂₂, R₂₉, R₃₀, R₄₈, R₄₉, R₆₃, R₆₈=are independently N or absent;
R₄₃, R₆₂, R₆₅, R₇₀=are independently A, C, U or absent;
R₁₅, R₂₇, R₃₃, R₃₄, R₄₀, R₅₁, R₅₂=are independently A, G or absent;
R₂, R₇, R₁₂, R₄₅, R₅₀, R₅₈, R₆₆=are independently A, G, U or absent;
R₃₁=A, U or absent;
R₃₂, R₄₄, R₆₀=are independently C, G or absent;
R₃, R₁₃, R₃₇, R₄₂, R₆₄, R₆₇=are independently C, G, U or absent;
R₆, R₁₁, R₂₈, R₃₅, R₅₅, R₅₉, R₆₁=are independently C, U or absent;
R₉, R₁₀, R₁₉, R₂₀=are independently G or absent;
R₁, R₂₁, R₃₉, R₇₂=are independently G, U or absent;
R₈, R₁₆, R₃₆, R₃₈, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I_{GLU} (SEQ ID NO: 580),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀=absent;
R₃₄, R₄₃, R₆₈, R₆₉=are independently A, C, G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄₅, R₄₆, R₄₈, R₅₀, R₅₁, R₅₅, R_{6 3}, R₆₄, R₆₅, R₆₆, R₇₀, R₇₁=are independently N or absent;
R₁₃, R₁₇, R₂₃, R₆₁=are independently A, C, U or absent;
R₁₀, R₁₄, R₂₄, R₄₀, R₅₂, R₅₆=are independently A, G or absent;
R₇, R₁₅, R₂₅, R₆₇, R₇₂=are independently A, G, U or absent;
R₁₁, R₅₇=are independently A, U or absent;
R₃₉=C, G or absent;
R₃, R₄, R₂₂, R₄₂, R₄₉, R₅₅, R₆₂=are independently C, G, U or absent;
R₁₈, R₂₈, R₃₅, R₃₇, R₅₃, R₅₉, R₆₀=are independently C, U or absent;
R₁₉=G or absent;
R₈, R₃₆, R₃₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLU} (SEQ ID NO: 581),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀, R₁₈, R₂₃=are absent
R₁₇, R₄₀=are independently A or absent;
R₂₆, R₂₇, R₃₄, R₄₃, R₆₈, R₆₉, R₇₁=are independently A, C, G or absent;
R₁, R₂, R₅, R₁₂, R₂₁, R₃₁, R₃₃, R₄₁, R₄₅, R₄₈, R₅₁, R₅₈, R₆₆, R₇₀=are independently N or absent;
R₄₄, R₆₁=are independently A, C, U or absent;
R₉, R₁₄, R₂₄, R₂₅, R₅₂, R₅₆, R₆₃=are independently A, G or absent;
R₇, R₁₅, R₄₆, R₅₀, R₆₇, R₇₂=are independently A, G, U or absent;
R₂₉, R₅₇=are independently A, U or absent;
R₆₀=C or absent;
R₃₉=C, G or absent;
R₃, R₆, R₂₀, R₃₀, R₃₂, R₄₂, R₅₅, R₆₂, R₆₅=are independently C, G, U or absent;
R₄, R₈, R₁₆, R₂₈, R₃₅, R₃₇, R₄₉, R₅₃, R₅₉=are independently C, U or absent;
R₁₀, R₁₉=are independently G or absent;
R₂₂, R₆₄=are independently G, U or absent;
R₁₁, R₁₃, R₃₆, R₃₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLU} (SEQ ID NO: 582),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀, R₁₇, R₁₈, R₂₃=are absent
R₁₄, R₂₇, R₄₀, R₇₁=are independently A or absent;
R₄₄=A, C or absent;
R₄₃=A, C, G or absent;
R₁, R₃₁, R₃₃, R₄₅, R₅₁, R₆₆=are independently N or absent;
R₂₁, R₄₁=are independently A, C, U or absent;
R₇, R₂₄, R₂₅, R₅₀, R₅₂, R₅₆, R₆₃, R₆₈, R₇₀=are independently A, G or absent;
R₅, R₄₆=are independently A, G, U or absent;
R₂₉, R₅₇, R₆₇, R₇₂=are independently A, U or absent;
R₂, R₃₉, R₆₀=are independently C or absent;
R₃, R₁₂, R₂₀, R₂₆, R₃₄, R₆₉=are independently C, G or absent;
R₆, R₃₀, R₄₂, R₄₈, R₆₅=are independently C, G, U or absent;
R₄, R₁₆, R₂₈, R₃₅, R₃₇, R₄₉, R₅₃, R₅₅, R₅₈, R₆₁, R₆₂=are independently C, U or absent;
R₉, R₁₀, R₁₉, R₆₄=are independently G or absent;
R₁₂, R₂₂, R₃₂=are independently G, U or absent;
R₈, R₁₁, R₁₃, R₃₆, R₃₈, R₅₄, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I_{GLY} (SEQ ID NO: 583),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀=absent;
R₂₄=A or absent;
R₃, R₉, R₄₀, R₅₀, R₅₁=are independently A, C, G or absent;
R₄, R₅, R₆, R₇, R₁₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄₅, R₄₆, R₄₅, R₄₉, R₅₈, R₆₃, R₆₄, R₆₅, R₆₆, R₆₇, R₆₈=are independently N or absent;
R₅₉=A, C, U or absent;
R₁, R₁₀, R₁₄, R₁₅, R₂₇, R₅₆=are independently A, G or absent;
R₂₀, R₂₅=are independently A, G, U or absent;
R₅₇, R₇₂=are independently A, U or absent;
R₃₈, R₃₉, R₆₀=are independently C or absent;
R₅₂=C, G or absent;
R₂, R₁₉, R₃₇, R₅₄, R₅₅, R₆₁, R₆₂, R₆₉, R₇₀=are independently C, G, U or absent;
R₁₁, R₁₃, R₁₇, R₂₈, R₃₅, R₃₆, R₇₁=are independently C, U or absent;
R₈, R₁₈, R₂₃, R₅₃=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{GLY} (SEQ ID NO: 584),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₆₈=are independently A, C, G or absent;
R₅, R₃₀, R₄₁, R₄₂, R₄₄, R₄₉, R₆₇=are independently A, C, G, U or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₁₆, R₂₂, R₂₅, R₂₉, R₄₆=are independently A, G, U or absent;
R₅₇=A, U or absent;
R₁₇, R₃₈, R₃₉, R₆₀, R₆₁, R₇₁=are independently C or absent;
R₆, R₅₂, R₆₄, R₆₆=are independently C, G or absent;
R₂, R₄, R₃₇, R₄₈, R₅₅, R₆₅=are independently C, G, U or absent;
R₁₃, R₃₅, R₄₃, R₆₂, R₆₉=are independently C, U or absent;
R₁, R₁₉, R₂₀, R₅₁, R₇₀=are independently G or absent;
R₂₁, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{GLY} (SEQ ID NO: 585),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₄₉, R₆₈=are independently A, C, G or absent;
R₅, R₃₀, R₄₁, R₄₄, R₆₇=are independently N or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₂₅, R₂₉, R₄₂, R₄₆=are independently A, G, U or absent;
R₁₆, R₅₇=are independently A, U or absent;
R₁₇, R₃₈, R₃₉, R₆₀, R₆₁, R₇₁=are independently C or absent;
R₆, R₅₂, R₆₄, R₆₆=are independently C, G or absent;
R₃₇, R₄₈, R₆₅=are independently C, G, U or absent;
R₂, R₄, R₁₃, R₃₅, R₄₃, R₅₅, R₆₂, R₆₉=are independently C, U or absent;
R₁, R₁₉, R₂₀, R₅₁, R₇₀=are independently G or absent;
R₂₁, R₂₂, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I_{HIS} (SEQ ID NO: 586),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₂₃=absent;
R₁₄, R₂₄, R₅₇=are independently A or absent;
R₇₂=A, C or absent;
R₉, R₂₇, R₄₃, R₄₈, R₆₉=are independently A, C, G or absent;
R₃, R₄, R₅, R₆, R₁₂, R₂₅, R₂₆, R₂₉, R₃₀, R₃₁, R₃₄, R₄₂, R₄₅, R₄₆, R₄₉, R₅₀, R₅₈, R₆₂, R₆₃, R₆₆, R₆₇, R₆₈=are independently N or absent;
R₁₃, R₂₁, R₄₁, R₄₄, R₆₅=are independently A, C, U or absent;
R₄₀, R₅₁, R₅₆, R₇₀=are independently A, G or absent;
R₇, R₃₂=are independently A, G, U or absent;
R₅₅, R₆₀=are independently C or absent;
R₁₁, R₁₆, R₃₃, R₆₄=are independently C, G, U or absent;
R₂, R₁₇, R₂₂, R₂₈, R₃₅, R₅₃, R₅₉, R₆₁, R₇₁=are independently C, U or absent;
R₁, R₁₀, R₁₅, R₁₉, R₂₀, R₃₇, R₃₉, R₅₂=are independently G or absent;
R₀=G, U or absent;
R₈, R₁₈, R₃₆, R₃₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{HIS} (SEQ ID NO: 587),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀, R₁₇, R₁₈, R₂₃=are absent;
R₇, R₁₂, R₁₄, R₂₄, R₂₇, R₄₅, R₅₇, R₅₈, R₆₃, R₆₇, R₇₂=are independently A or absent;
R₃=A, C, U or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₂₅=C, G or absent;
R₉=C, G, U or absent;
R₈, R₁₃, R₂₆, R₃₃, R₃₅, R₅₀, R₅₃, R₆₁, R₆₈=are independently C, U or absent;
R₁, R₆, R₁₀, R₁₅, R₁₉, R₂₀, R₃₂, R₃₄, R₃₇, R₃₉, R₄₀, R₄₆, R₅₁, R₅₂, R₆₂, R₆₄, R₆₉=are independently G or
absent;
R₁₆=G, U or absent;
R₅, R₁₁, R₂₁, R₂₂, R₂₉, R₃₁, R₃₆, R₃₈, R₅₄, R₅₉, R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{HIS} (SEQ ID NO: 588),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀, R₁₇, R₁₈, R₂₃=are absent
R₇, R₁₂, R₁₄, R₂₄, R₂₇, R₄₅, R₅₇, R₅₈, R₆₃, R₆₇, R₇₂=are independently A or absent;
R₃=A, C or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₈, R₉, R₂₆, R₃₃, R₃₅, R₅₀, R₆₁, R₆₈=are independently C, U or absent;
R₁, R₆, R₁₀, R₁₅, R₁₉, R₂₀, R₂₅, R₃₂, R₃₄, R₃₇, R₃₉, R₄₀, R₄₆, R₅₁, R₅₂, R₆₂, R₆₄, R₆₉=are independently G or absent;
R₅, R₁₁, R₁₃, R₁₆, R₂₁, R₂₂, R₂₉, R₃₁, R₃₆, R₃₈, R₅₃, R₅₄, R₅₉, R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I_{ILE} (SEQ ID NO: 589),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₂₃=absent;
R₃₈, R₄₁, R₅₇, R₇₂=are independently A or absent;
R₁, R₂₆=are independently A, C, G or absent;
R₀, R₃, R₄, R₆, R₁₆, R₃₁, R₃₂, R₃₄, R₃₇, R₄₂, R₄₃, R₄₄, R₄₅, R₄₆, R₄₈, R₄₉, R₅₀, R₅₅, R₅₉, R₆₂, R₆₃, R₆₄, R₆₆, R₆₇, R₆₈, R₆₉=are independently N or absent;
R₂₂, R₆₁, R₆₅=are independently A, C, U or absent;
R₉, R₁₄, R₁₅, R₂₄, R₂₇, R₄₀=are independently A, G or absent;
R₇, R₂₅, R₂₉, R₅₁, R₅₆=are independently A, G, U or absent;
R₁₈, R₅₄=are independently A, U or absent;
R₆₀=C or absent;
R₂, R₅₂, R₇₀=are independently C, G or absent;
R₅, R₁₂, R₂₁, R₃₀, R₃₃, R₇₁=are independently C, G, U or absent;
R₁₁, R₁₃, R₁₇, R₂₈, R₃₅, R₅₃, R₅₅=are independently C, U or absent;
R₁₀, R₁₉, R₂₀=are independently G or absent;
R₈, R₃₆, R₃₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ILE (SEQ ID NO: 590),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀, R₁₈, R₂₃=are absent
R₂₄, R₃₈, R₄₀, R₄₁, R₅₇, R₇₂=are independently A or absent;
R₂₆, R₆₅=are independently A, C or absent;
R₅₈, R₅₉, R₆₇=are independently N or absent;
R₂₂=A, C, U or absent;
R₆, R₉, R₁₄, R₁₅, R₂₉, R₃₄, R₄₃, R₄₆, R₄₈, R₅₀, R₅₁, R₆₃, R₆₉=are independently A, G or absent;
R₃₇, R₅₆=are independently A, G, U or absent;
R₅₄=A, U or absent;
R₂₈, R₃₅, R₆₀, R₆₂, R₇₁=are independently C or absent;
R₂, R₅₂, R₇₀=are independently C, G or absent;
R₅=C, G, U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆=are independently C,U or absent;
R₁, R₁₀, R₁₉, R₂₀, R₂₅, R₂₇, R₃₁, R₆₈=are independently G or absent;
R₇, R₁₂, R₃₂=are independently G, U or absent;
R₈, R₁₆, R₃₃, R₃₆, R₃₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ILE (SEQ ID NO: 591),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀, R₁₈, R₂₃=are absent
R₁₄, R₂₄, R₃₈, R₄₀, R₄₁, R₅₇, R₇₂=are independently A or absent;
R₂₆, R₆₅=are independently A, C or absent;
R₂₂, R₅₉=are independently A, C, U or absent;
R₆, R₉, R₁₅, R₃₄, R₄₃, R₄₆, R₅₁, R₅₆, R₆₃, R₆₉=are independently A, G or absent;
R₃₇=A, G, U or absent;
R₁₃, R₂₈, R₃₅, R₄₄, R₅₅, R₆₀, R₆₂, R₇₁=are independently C or absent;
R₂, R₅, R₇₀=are independently C, G or absent;
R₅₈, R₆₇=are independently C, G, U or absent;
R₃, R₄, R₁₁, R₁₇, R₂₁, R₃₀, R₄₂, R₄₅, R₄₉, R₅₃, R₆₁, R₆₄, R₆₆=are independently C, U or absent;
R₁, R₁₀, R₁₉, R₂₀, R₂₅, R₂₇, R₂₉, R₃₁, R₃₂, R₄₈, R₅₀, R₅₂, R₆₈=are independently G or absent;
R₇, R₁₂=are independently G, U or absent;
R₈, R₁₆, R₃₃, R₃₆, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I_{MET} (SEQ ID NO: 592),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₂₃=are absent;
R₁₄, R₃₈, R₄₀, R₅₇=are independently A or absent;
R₆₀=A, C or absent;
R₃₃, R₄₈, R₇₀=are independently A, C, G or absent;
R₁, R₃, R₄, R₅, R₆, R₁₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₅, R_{6 2}, R₆₃, R₆₆, R₆₇, R₆₈, R₆₉, R₇₁=are independently N or absent;
R₁₈, R₃₅, R₄₁, R₅₉, R₆₅=are independently A, C, U or absent;
R₉, R₁₅, R₅₁=are independently A, G or absent;
R₇, R₂₄, R₂₅, R₃₄, R₅₃, R₅₆=are independently A, G, U or absent;
R₇₂=A, U or absent;
R₃₇=C or absent;
R₁₀, R₅₅=are independently C, G or absent;
R₂, R₁₃, R₂₈, R₄₃, R₆₄=are independently C, G, U or absent;
R₃₆, R₆₁=are independently C, U or absent;
R₁₉, R₂₀, R₅₂=are independently G or absent;
R₈, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{MET} (SEQ ID NO: 593),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₀, R₆₂, R₆₅=are independently A, C or absent;
R₆, R₄₅, R₆₇=are independently A, C, G or absent;
R₄=N or absent;
R₂₁, R₄₂=are independently A, C, U or absent;
R₁, R₉, R₂₇, R₂₉, R₃₂, R₄₆, R₅₁=are independently A, G or absent;
R₁₇, R₄₉, R₅₃, R₅₆, R₅₈=are independently A, G, U or absent;
R₆₃=A, U or absent;
R₃, R₁₃, R₃₇=are independently C or absent;
R₄₈, R₅₅, R₆₄, R₇₀=are independently C, G or absent;
R₂, R₅, R₆₆, R₆₈=are independently C, G, U or absent;
R₁₁, R₁₆, R₂₆, R₂₈, R₃₀, R₃₁, R₃₅, R₃₆, R₄₃, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀=are independently G, U or absent;
R₈, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{MET} (SEQ ID NO: 594),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₂, R₆₅=are independently A, C or absent;
R₆, R₆₇=are independently A, C, G or absent;
R₄, R₂₁=are independently A, C, U or absent;
R₁, R₉, R₂₇, R₂₉, R₃₂, R₄₅, R₄₆, R₅₁=are independently A, G or absent;
R₁₇, R₅₆, R₅₈=are independently A, G, U or absent;
R₄₉, R₅₃, R₆₃=are independently A, U or absent;
R₃, R₁₃, R₂₆, R₃₇, R₄₃, R₆₀=are independently C or absent;
R₂, R₄₈, R₅₅, R₆₄, R₇₀=are independently C, G or absent;
R₅, R₆₆=are independently C, G, U or absent;
R₁₁, R₁₆, R₂₈, R₃₀, R₃₁, R₃₅, R₃₆, R₄₂, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀, R₆₈=are independently G, U or absent;
R₈, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I_{LEU} (SEQ ID NO: 595),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent;
R₃₈, R₅₇=are independently A or absent;
R₆₀=A, C or absent;
R₁, R₁₃, R₂₇, R₄₈, R₅₁, R₅₆=are independently A, C, G or absent;
R₂, R₃, R₄, R₅, R₆, R₇, R₉, R₁₀, R₁₁, R₁₂, R₁₆, R₂₃, R₂₆, R₂₈, R₂₉, R₃₀, R₃₁, R₃₂, R₃₃, R₃₄, R₃₇, R₄₁, R₄₂, R₄₃, R₄₄, R₄₅, R₄₆, R₄₉, R₅₀, R₅₈, R₆₂, R₆₃, R₆₅, R₆₆, R₆₇, R₆₈, R₆₉, R₇₀=are independently N or absent;
R₁₇, R₁₈, R₂₁, R₂₂, R₂₅, R₃₅, R₅₅=are independently A, C, U or absent;
R₁₄, R₁₅, R₃₉, R₇₂=are independently A, G or absent;
R₂₄, R₄₀=are independently A, G, U or absent;
R₅₂, R₆₁, R₆₄, R₇₁=are independently C, G, U or absent;
R₃₆, R₅₃, R₅₉=are independently C, U or absent;
R₁₉=G or absent;
R₂₀=G, U or absent;
R₈, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LEU} (SEQ ID NO: 596),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₆₉=are independently A, C, G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂₁, R₂₂, R₂₈, R₃₁, R₃₇, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₂₆, R₃₀, R₅₂, R₆₂, R₆₄, R₆₅, R₆₇, R₆₈=are independently C, G, U or absent;
R₁₈, R₃₅, R₄₂, R₅₃, R₅₉, R₆₁, R₇₁=are independently C, U or absent;
R₁₉, R₅₁=are independently G or absent;
R₁₀, R₂₀=are independently G, U or absent;
R₈, R₂₃, R₃₆, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LEU} (SEQ ID NO: 597),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₅₈, R₆₉=are independently A, C, G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₂₈, R₃₁, R₃₇, R₄₁, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₃₀, R₅₂, R₆₂, R₆₄, R₆₇, R₆₈=are independently C, G, U or absent;
R₁₈, R₃₅, R₄₂, R₅₃, R₅₉, R₆₁, R₆₅, R₇₁=are independently C, U or absent;
R₁₉, R₅₁=are independently G or absent;
R₁₀, R₂₀, R₂₆=are independently G, U or absent;
R₈, R₂₃, R₃₆, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I_{L}ys (SEQ ID NO: 598),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀=absent
R₁₄=A or absent;
R₄₀, R₄₁=are independently A, C or absent;
R₃₄, R₄₃, R₅₁=are independently A, C, G or absent;
R₁, R₂, R₃, R₄, R₅, R₆, R₇, R₁₁, R₁₂, R₁₆, R₂₁, R₂₆, R₃₀, R₃₁, R₃₂, R₄₄, R₄₅, R₄₆, R₄₈, R₄₉, R₅₀, R₅₅, R₆₂, R₆₃, R₆₅, R₆₆, R₆₇, R₆₈, R₆₉, R₇₀=are independently N or absent;
R₁₃, R₁₇, R₅₉, R₇₁=are independently A, C, U or absent;
R₉, R₁₅, R₁₉, R₂₀, R₂₅, R₂₇, R₅₂, R₅₆=are independently A, G or absent;
R₂₄, R₂₉, R₇₂=are independently A, G, U or absent;
R₁₈, R₅₇=are independently A, U or absent;
R₁₀, R₃₃=are independently C, G or absent;
R₄₂, R₆₁, R₆₄=are independently C, G, U or absent;
R₂₈, R₃₅, R₃₆, R₃₇, R₅₃, R₅₅, R₆₀=are independently C, U or absent;
R₈, R₂₂, R₂₃, R₃₈, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{LYS} (SEQ ID NO: 599),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀, R₁₈, R₂₃=are absent
R₁₄=A or absent;
R₄₀, R₄₁, R₄₃=are independently A, C or absent;
R₃, R₇=are independently A, C, G or absent;
R₁, R₆, R₁₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A, C, U or absent;
R₉, R₁₅, R₁₉, R₂₀, R₂₅, R₂₇, R₃₄, R₅₀, R₅₂, R₅₆, R₇₀, R₇₂=are independently A, G or absent;
R₅, R₂₄, R₂₆, R₂₉, R₃₂, R₄₆, R₆₉=are independently A, G, U or absent; R₅₇=A, U or absent;
R₁₀, R₆₁=are independently C, G or absent;
R₄, R₁₆, R₂₁, R₃₀, R₅₈, R₆₄=are independently C, G, U or absent;
R₂₈, R₃₅, R₃₆, R₃₇, R₄₂, R₅₃, R₅₅, R₅₉, R₆₀, R₆₂=are independently C, U or absent;
R₃₃, R₅₁=are independently G or absent;
R₈=G, U or absent;
R₂₂, R₃₈, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{LYS} (SEQ ID NO: 600),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀, R₁₈, R₂₃=absent
R₉, R₁₄, R₃₄, R₄₁=are independently A or absent;
R₄₀=A, C or absent;
R₁, R₃, R₇, R₃₁=are independently A, C, G or absent;
R₄₈, R₆₅, R₆₈=are independently N or absent;
R₂, R₁₃, R₁₇, R₄₄, R₆₃, R₆₆=are independently A, C, U or absent;
R₅, R₁₅, R₁₉, R₂₀, R₂₅, R₂₇, R₂₉, R₅₀, R₅₂, R₅₆, R₇₀, R₇₂=are independently A, G or absent;
R₆, R₂₄, R₃₂, R₄₉=are independently A, G, U or absent;
R₁₂, R₂₆, R₄₆, R₅₇=are independently A, U or absent;
R₁₁, R₂₈, R₃₅, R₄₃=are independently C or absent;
R₁₀, R₄₅, R₆₁=are independently C, G or absent;
R₄, R₂₁, R₆₄=are independently C, G, U or absent;
R₃₇, R₅₃, R₅₅, R₅₉, R₆₀, R₆₂, R₆₇, R₇₁=are independently C, U or absent;
R₃₃, R₅₁=are independently G or absent;
R₈, R₃₀, R₅₈, R₆₉=are independently G, U or absent;
R₁₆, R₂₂, R₃₆, R₃₈, R₃₉, R₄₂, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I PHE (SEQ ID NO: 601),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₂₃=are absent
R₉, R₁₄, R₃₈, R₃₉, R₅₇, R₇₂=are independently A or absent;
R₇₁=A, C or absent;
R₄₁, R₇₀=are independently A, C, G or absent;
R₄, R₅, R₆, R₃₀, R₃₁, R₃₂, R₃₄, R₄₂, R₄₄, R₄₅, R₄₆, R₄₈, R₄₉, R₅₅, R₆₂, R₆₃, R₆₆, R₆₇, R₆₈, R₆₉=are independently N or absent;
R₁₆, R₆₁, R₆₅=are independently A, C, U or absent;
R₁₅, R₂₆, R₂₇, R₂₉, R₄₀, R₅₆=are independently A, G or absent;
R₇, R₅₁=are independently A, G, U or absent;
R₂₂, R₂₄=are independently A, U or absent;
R₅₅, R₆₀=are independently C or absent;
R₂, R₃, R₂₁, R₃₃, R₄₃, R₅₀, R₆₄=are independently C, G, U or absent;
R₁₁, R₁₂, R₁₃, R₁₇, R₂₈, R₃₅, R₃₆, R₅₉=are independently C, U or absent;
R₁₀, R₁₉, R₂₀, R₂₅, R₃₇, R₅₂=are independently G or absent;
R₁=G, U or absent;
R₈, R₁₈, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PHE (SEQ ID NO: 602),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₁₈, R₂₃=absent
R₁₄, R₂₄, R₃₈, R₃₉, R₅₇, R₇₂=are independently A or absent;
R₄₆, R₇₁=are independently A, C or absent;
R₄, R₇₀=are independently A, C, G or absent;
R₄₅=A, C, U or absent;
R₆, R₇, R₁₅, R₂₆, R₂₇, R₃₂, R₃₄, R₄₀, R₄₁, R₅₆, R₆₉=are independently A, G or absent;
R₂₉=A, G, U or absent;
R₅, R₉, R₆₇=are independently A, U or absent;
R₃₅, R₄₉, R₅₅, R₆₀=are independently C or absent;
R₂₁, R₄₃, R₆₂=are independently C, G or absent;
R₂, R₃₃, R₆₈=are independently C, G, U or absent;
R₃, R₁₁, R₁₂, R₁₃, R₂₈, R₃₀, R₃₆, R₄₂, R₄₄, R₄₈, R₅₈, R₅₉, R₆₁, R₆₆=are independently C, U or absent;
R₁₀, R₁₉, R₂₀, R₂₅, R₃₇, R₅₁, R₅₂, R₆₃, R₆₄=are independently G or absent;
R₁, R₃₁, R₅₀=are independently G, U or absent;
R₈, R₁₆, R₁₇, R₂₂, R₅₃, R₅₄, R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₁₈, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A, C or absent;
R₇₀=A, C, G or absent;
R₄, R₆, R₁₅, R₅₆, R₆₉=are independently A, G or absent;
R₉, R₄₅=are independently A, U or absent;
R₂, R₁₁, R₁₃, R₃₅, R₄₃, R₄₉, R₅₅, R₆₀, R₆₈, R₇₁=are independently C or absent;
R₃₃=C, G or absent;
R₃, R₂₈, R₃₆, R₄₈, R₅₈, R₅₉, R₆₁=are independently C, U or absent;
R₁, R₁₀, R₁₉, R₂₀, R₂₁, R₂₅, R₂₇, R₂₉, R₃₇, R₄₀, R₅₁, R₅₂, R₆₂, R₆₃, R₆₄=are independently G or absent;
R₈, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I_{PRO} (SEQ ID NO: 604),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀=absent
R₁₄, R₅₇=are independently A or absent;
R₇₀, R₇₂=are independently A, C or absent;
R₉, R₂₆, R₂₇=are independently A, C, G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₈, R₄₉, R₅₀, R₅₅, R₆₁, R₆₂, R₆₃, R₆₄, R₆₆, R₆₇, R₆₈=are independently N or absent;
R₃₅, R₆₅=are independently A, C, U or absent;
R₂₄, R₄₀, R₅₆=are independently A, G or absent;
R₇, R₂₅, R₅₁=are independently A, G, U or absent;
R₅₅, R₆₀=are independently C or absent;
R₁, R₃, R₇₁=are independently C, G or absent;
R₁₁, R₁₂, R₂₀, R₆₉=are independently C, G, U or absent;
R₁₃, R₁₇, R₁₈, R₂₂, R₂₃, R₂₈, R₅₉=are independently C, U or absent;
R₁₀, R₁₅, R₁₉, R₃₈, R₃₉, R₅₂=are independently G or absent;
R₂=are independently G, U or absent;
R₈, R₃₆, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{PRO} (SEQ ID NO: 605),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀, R₁₇, R₁₈, R₂₂, R₂₃=absent;
R₁₄, R₄₅, R₅₆, R₅₇, R₅₈, R₆₅, R₆₈=are independently A or absent;
R₆₁=A, C, G or absent;
R₄₃=N or absent;
R₃₇=A, C, U or absent;
R₂₄, R₂₇, R₃₃, R₄₀, R₄₄, R₆₃=are independently A, G or absent;
R₃, R₁₂, R₃₀, R₃₂, R₄₈, R₅₅, R₆₀, R₇₀, R₇₁, R₇₂=are independently C or absent;
R₅, R₃₄, R₄₂, R₆₆=are independently C, G or absent;
R₂₀=C, G, U or absent;
R₃₅, R₄₁, R₄₉, R₆₂=are independently C, U or absent;
R₁, R₂, R₆, R₉, R₁₀, R₁₅, R₁₉, R₂₆, R₃₈, R₃₉, R₄₆, R₅₀, R₅₁, R₅₂, R₆₄, R₆₇, R₆₉=are independently G or absent;
R₁₁, R₁₆=are independently G, U or absent;
R₄, R₇, R₈, R₁₃, R₂₁, R₂₅, R₂₈, R₂₉, R₃₁, R₃₆, R₅₃, R₅₄, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{PRO} (SEQ ID NO: 606),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀, R₁₇, R₁₈, R₂₂, R₂₃=absent
R₁₄, R₄₅, R₅₆, R₅₇, R₅₈, R₆₅, R₆₈=are independently A or absent;
R₃₇=A, C, U or absent;
R₂₄, R₂₇, R₄₀=are independently A, G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C, G or absent;
R₄₃=C, G, U or absent;
R₄₁=C, U or absent;
R₁, R₂, R₆, R₉, R₁₀, R₁₅, R₁₉, R₂₀, R₂₆, R₃₃, R₃₈, R₃₉, R₄₄, R₄₆, R₅₀, R₅₁, R₅₂, R₆₃, R₆₄, R₆₇, R₆₉=are independently G or absent;
R₁₆=G, U or absent;
R₄, R₇, R₈, R₁₁, R₁₃, R₂₁, R₂₅, R₂₈, R₂₉, R₃₁, R₃₅, R₃₆, R₅₃, R₅₄, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I_{SER} (SEQ ID NO: 607),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀=absent;
R₁₄, R₂₄, R₅₇=are independently A or absent;
R₄₁=A, C or absent;
R₂, R₃, R₄, R₅, R₆, R₇, R₉, R₁₀, R₁₁, R₁₂, R₁₃, R₁₆, R₂₁, R₂₅, R₂₆, R₂₇, R₂₈, R₃₀, R₃₁, R₃₂, R₃₃, R₃₄, R₃₇, R₄₂, R₄₃, R₄₄, R₄₅, R₄₆, R₄₈, R₄₉, R₅₀, R₆₂, R₆₃, R₆₄, R₆₅, R₆₆, R₆₇, R₆₈, R₆₉, R₇₀=are independently N or absent;
R₁₈=A, C, U or absent;
R₁₅, R₄₀, R₅₁, R₅₆=are independently A, G or absent;
R₁, R₂₉, R₅₅, R₇₂=are independently A, G, U or absent;
R₃₉=A, U or absent;
R₆₀=C or absent;
R₃₈=C, G or absent;
R₁₇, R₂₂, R₂₃, R₇₁=are independently C, G, U or absent;
R₈, R₃₅, R₃₆, R₅₅, R₅₉, R₆₁=are independently C, U or absent;
R₁₉, R₂₀=are independently G or absent;
R₅₂=G, U or absent;
R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{SER} (SEQ ID NO: 608),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀, R₂₃=absent
R₁₄, R₂₄, R₄₁, R₅₇=are independently A or absent;
R₄₄=A, C or absent;
R₂₅, R₄₅, R₄₈=are independently A, C, G or absent;
R₂, R₃, R₄, R₅, R₃₇, R₅₀, R₆₂, R₆₆, R₆₇, R₆₉, R₇₀=are independently N or absent;
R₁₂, R₂₈, R₆₅=are independently A, C, U or absent;
R₉, R₁₅, R₂₉, R₃₄, R₄₀, R₅₆, R₆₃=are independently A, G or absent;
R₇, R₂₆, R₃₀, R₃₃, R₄₆, R₅₈, R₇₂=are independently A, G, U or absent;
R₃₉=A, U or absent;
R₁₁, R₃₅, R₆₀, R₆₁=are independently C or absent;
R₁₃, R₃₈=are independently C, G or absent;
R₆, R₁₇, R₃₁, R₄₃, R₆₄, R₆₈=are independently C, G, U or absent;
R₃₆, R₄₂, R₄₉, R₅₅, R₅₉, R₇₁=are independently C, U or absent;
R₁₀, R₁₉, R₂₀, R₂₇, R₅₁=are independently G or absent;
R₁, R₁₆, R₃₂, R₅₂=are independently G, U or absent;
R₈, R₁₈, R₂₁, R₂₂, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{SER} (SEQ ID NO: 609),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀, R₂₃=absent
R₁₄, R₂₄, R₄₁, R₅₇, R₅₈=are independently A or absent;
R₄₄=A, C or absent;
R₂₅, R₄₈=are independently A, C, G or absent;
R₂, R₃, R₅, R₃₇, R₆₆, R₆₇, R₆₉, R₇₀=are independently N or absent;
R₁₂, R₂₈, R₆₂=are independently A, C, U or absent;
R₇, R₉, R₁₅, R₂₉, R₃₃, R₃₄, R₄₀, R₄₅, R₅₆, R₆₃=are independently A, G or absent;
R₄, R₂₆, R₄₆, R₅₀=are independently A, G, U or absent;
R₃₀, R₃₉=are independently A, U or absent;
R₁₁, R₁₇, R₃₅, R₆₀, R₆₁=are independently C or absent;
R₁₃, R₃₈=are independently C, G or absent;
R₆, R₆₄=are independently C, G, U or absent;
R₃₁, R₄₂, R₄₃, R₄₉, R₅₅, R₅₉, R₆₅, R₆₈, R₇₁=are independently C, U or absent;
R₁₀, R₁₉, R₂₀, R₂₇, R₅₁, R₅₂=are independently G or absent;
R₁, R₁₆, R₃₂, R₇₂=are independently G, U or absent;
R₈, R₁₈, R₂₁, R₂₂, R₃₆, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I_{THR} (SEQ ID NO: 610),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₂₃=absent
R₁₄, R₄₁, R₅₇=are independently A or absent;
R₅₆, R₇₀=are independently A, C, G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₈, R₄₉, R₅₀, R₅₅, R₆₂, R₆₃, R₆₄, R₆₅, R₆₆, R₆₇, R₆₈, R₇₂=are independently N or absent;
R₁₃, R₁₇, R₂₁, R₃₅, R₆₁=are independently A, C, U or absent;
R₁, R₉, R₂₄, R₂₇, R₂₉, R₆₉=are independently A, G or absent;
R₁₅, R₂₅, R₅₁=are independently A, G, U or absent;
R₄₀, R₅₃=are independently A, U or absent;
R₃₃, R₄₃=are independently C, G or absent;
R₂, R₃, R₅₉=are independently C, G, U or absent;
R₁₁, R₁₈, R₂₂, R₂₈, R₃₆, R₅₄, R₅₅, R₆₀, R₇₁=are independently C, U or absent;
R₁₀, R₂₀, R₃₈, R₅₂=are independently G or absent;
R₁₉=G, U or absent;
R₈, R₃₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{THR} (SEQ ID NO: 611),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₁₈, R₂₃=absent
R₁₄, R₄₁, R₅₇=are independently A or absent;
R₉, R₄₂, R₄₄, R₄₈, R₅₆, R₇₀=are independently A, C, G or absent;
R₄, R₆, R₁₂, R₂₆, R₄₉, R₅₈, R₆₃, R₆₄, R₆₆, R₆₈=are independently N or absent;
R₁₃, R₂₁, R₃₁, R₃₇, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₇=are independently A, G, U or absent;
R₄₀, R₅₃=are independently A, U or absent;
R₃₅=C or absent;
R₃₃, R₄₃=are independently C, G or absent;
R₂, R₃, R₅, R₁₆, R₃₂, R₃₄, R₅₉, R₆₅, R₇₂=are independently C, G, U or absent;
R₁₁, R₁₇, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₈, R₃₉, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{THR} (SEQ ID NO: 612),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₁₈, R₂₃=absent
R₁₄, R₄₀, R₄₁, R₅₇=are independently A or absent;
R₄₄=A, C or absent;
R₉, R₄₂, R₄₈, R₅₆=are independently A, C, G or absent;
R₄, R₆, R₁₂, R₂₆, R₅₈, R₆₄, R₆₆, R₆₈=are independently N or absent;
R₁₃, R₂₁, R₃₁, R₃₇, R₄₉, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₃, R₆₇=are independently A, G, U or absent;
R₅₃=A, U or absent;
R₃₅=C or absent;
R₂, R₃₃, R₄₃, R₇₀=are independently C, G or absent;
R₅, R₁₆, R₃₄, R₅₉, R₆₅=are independently C, G, U or absent;
R₃, R₁₁, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₃₂=G, U or absent;
R₈, R₁₇, R₃₉, R₅₄, R₇₂=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I_{TRP} (SEQ ID NO: 613),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A, C, G or absent;
R₄, R₅, R₆, R₂₉, R₃₀, R₃₁, R₃₂, R₃₄, R₄₂, R₄₄, R₄₅, R₄₆, R₄₉, R₅₁, R₅₈, R₆₃, R₆₆, R₆₇, R₆₈=are independently N or absent;
R₁₃, R₁₄, R₁₆, R₁₈, R₂₁, R₆₁, R₆₅, R₇₁=are independently A, C, U or absent;
R₁, R₉, R₁₀, R₁₅, R₃₃, R₅₀, R₅₆=are independently A, G or absent;
R₇, R₂₅, R₇₂=are independently A, G, U or absent;
R₃₇, R₃₈, R₅₅, R₆₀=are independently C or absent;
R₁₂, R₃₅, R₄₃, R₆₄, R₆₉, R₇₀=are independently C, G, U or absent;
R₁₁, R₁₇, R₂₂, R₂₈, R₅₉, R₆₂=are independently C, U or absent;
R₁₉, R₂₀, R₅₂=are independently G or absent;
R₈, R₂₃, R₃₆, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TRP} (SEQ ID NO: 614),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TRP} (SEQ ID NO: 615),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I_{TYR} (SEQ ID NO: 616),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₄-R₆₅-R₆₆-R₆₇-R₆₈-R₆₉-R₇₀-R₇₁-R₇₂

wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A, C, G or absent;
R₃, R₄, R₅, R₆, R₉, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₅, R₆₂, R₆₃, R₆₆,
R₆₇, R₆₈, R₆₉, R₇₀=are independently N or absent;
R₂₂, R₆₅=are independently A, C, U or absent;
R₁₅, R₂₄, R₂₇, R₃₃, R₃₇, R₄₀, R₅₆=are independently A, G or absent;
R₇, R₂₉, R₃₄, R₇₂=are independently A, G, U or absent;
R₂₃, R₅₃=are independently A, U or absent;
R₃₅, R₆₀=are independently C or absent;
R₂₀=C, G or absent;
R₁, R₂, R₂₈, R₆₁, R₆₄=are independently C, G, U or absent;
R₁₁, R₁₇, R₂₁, R₄₃, R₅₅=are independently C, U or absent;
R₁₉, R₅₂=are independently G or absent;
R₈, R₁₈, R₃₆, R₃₈, R₅₄, R₅₉=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{TYR} (SEQ ID NO: 617),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇=are independently A or absent;
R₄₄, R₆₉=are independently A, C or absent;
R₇₁=A, C, G or absent;
R₆₈=N or absent;
R₅₈=A, C, U or absent;
R₃₃, R₃₇, R₄₁, R₅₆, R₆₂, R₆₃=are independently A, G or absent;
R₆, R₂₉, R₇₂=are independently A, G, U or absent;
R₃₁, R₄₅, R₅₃=are independently A, U or absent;
R₁₃, R₃₅, R₄₉, R₆₀=are independently C or absent;
R₂₀, R₄₈, R₆₄, R₆₇, R₇₀=are independently C, G or absent;
R₁, R₂, R₅, R₁₆, R₆₆=are independently C, G, U or absent;
R₁₁, R₂₁, R₂₈, R₄₃, R₅₅, R₆₁=are independently C, U or absent;
R₁₀, R₁₅, R₁₉, R₂₅, R₂₇, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₄, R₅₉, R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TYR} (SEQ ID NO: 618),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇, R₇₂=are independently A or absent;
R₄₄, R₆₉=are independently A, C or absent;
R₇₁=A, C, G or absent;
R₃₇, R₄₁, R₅₆, R₆₂, R₆₃=are independently A, G or absent;
R₆, R₂₉, R₆₈=are independently A, G, U or absent;
R₃₁, R₄₅, R₅₈=are independently A, U or absent;
R₁₃, R₂₈, R₃₅, R₄₉, R₆₀, R₆₁=are independently C or absent;
R₅, R₄₈, R₆₄, R₆₇, R₇₀=are independently C, G or absent;
R₁, R₂=are independently C, G, U or absent;
R₁₁, R₁₆, R₂₁, R₄₃, R₅₅, R₆₆=are independently C, U or absent;
R₁₀, R₁₅, R₁₉, R₂₀, R₂₅, R₂₇, R₃₃, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₃, R₅₄, R₅₉, R₆₅=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I_{VAL} (SEQ ID NO: 619),

R₀-R₁-R₂-R₃-R₄-R₅-R₆-R₇-R₈-R₉-R₁₀-R₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₂₃=absent;
R₂₄, R₃₈, R₅₇=are independently A or absent;
R₉, R₇₂=are independently A, C, G or absent;
R₂, R₄, R₅, R₆, R₇, R₁₂, R₁₅, R₁₆, R₂₁, R₂₅, R₂₆, R₂₉, R₃₁, R₃₂, R₃₃, R₃₄, R₃₇, R₄₁, R₄₂, R₄₃, R₄₄, R₄₅, R₄₆, R₄₈, R_{4 9}, R₅₀, R₅₈, R₆₁, R₆₂, R₆₃, R₆₄, R₆₅, R₆₆, R₆₇, R₆₈, R₆₉, R₇₀=are independently N or absent;
R₁₇, R₃₅, R₅₉=are independently A, C, U or absent;
R₁₀, R₁₄, R₂₇, R₄₀, R₅₂, R₅₆=are independently A, G or absent;
R₁, R₃, R₅₁, R₅₃=are independently A, G, U or absent;
R₃₉=C or absent;
R₁₃, R₃₀, R₅₅=are independently C, G, U or absent;
R₁₁, R₂₂, R₂₈, R₆₀, R₇₁=are independently C, U or absent;
R₁₉=G or absent;
R₂₀=G, U or absent;
R₈, R₁₈, R₃₆, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{VAL} (SEQ ID NO: 620),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₁₈, R₂₃=absent;
R₂₄, R₃₈, R₅₇=are independently A or absent;
R₆₄, R₇₀, R₇₂=are independently A, C, G or absent;
R₁₅, R₁₆, R₂₆, R₂₉, R₃₁, R₃₂, R₄₃, R₄₄, R₄₅, R₄₉, R₅₀, R₅₈, R₆₂, R₆₅=are independently N or absent;
R₆, R₁₇, R₃₄, R₃₇, R₄₁, R₅₉=are independently A, C, U or absent;
R₉, R₁₀, R₁₄, R₂₇, R₄₀, R₄₆, R₅₁, R₅₂, R₅₆=are independently A, G or absent;
R₇, R₁₂, R₂₅, R₃₃, R₅₃, R₆₃, R₆₆, R₆₈=are independently A, G, U or absent;
R₆₉=A, U or absent;
R₃₉=C or absent;
R₅, R₆₇=are independently C, G or absent;
R₂, R₄, R₁₃, R₄₈, R₅₅, R₆₁=are independently C, G, U or absent;
R₁₁, R₂₂, R₂₈, R₃₀, R₃₅, R₆₀, R₇₁=are independently C, U or absent;
R₁₉=G or absent;
R₁, R₃, R₂₀, R₄₂=are independently G, U or absent;
R₈, R₂₁, R₃₆, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{VAL} (SEQ ID NO: 621),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₁₈, R₂₃=absent
R₂₄, R₃₈, R₄₀, R₅₇, R₇₂=are independently A or absent;
R₂₉, R₆₄, R₇₀=are independently A, C, G or absent;
R₄₉, R₅₀, R₆₂=are independently N or absent;
R₁₆, R₂₆, R₃₁, R₃₂, R₃₇, R₄₁, R₄₃, R₅₉, R₆₅=are independently A, C, U or absent;
R₉, R₁₄, R₂₇, R₄₆, R₅₂, R₅₆, R₆₆=are independently A, G or absent;
R₇, R₁₂, R₂₅, R₃₃, R₄₄, R₄₅, R₅₃, R₅₈, R₆₃, R₆₈=are independently A, G, U or absent;
R₆₉=A, U or absent;
R₃₉=C or absent;
R₅, R₆₇=are independently C, G or absent;
R₂, R₄, R₁₃, R₁₅, R₄₈, R₅₅=are independently C, G, U or absent;
R₆, R₁₁, R₂₂, R₂₈, R₃₀, R₃₄, R₃₅, R₆₀, R₆₁, R₇₁=are independently C, U or absent;
R₁₀, R₁₉, R₅₁=are independently G or absent;
R₁, R₃, R₂₀, R₄₂=are independently G, U or absent;
R₈, R₁₇, R₂₁, R₃₆, R₅₄=are independently U or absent;
[R₄₇]ₓ=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 10, e.g., any one of SEQ ID NOs: 452-561 disclosed in Table 10.

TABLE 10
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Method of Making TREMs, TREM Core Fragments, and TREM Fragments

In vitro methods for synthesizing oligonucleotides are known in the art and can be used to make a TREM, a TREM core fragment or a TREM fragment disclosed herein. For example, a TREM, TREM core fragment or TREM fragment can be synthesized using solid state synthesis or liquid phase synthesis.

In an embodiment, a TREM, a TREM core fragment or a TREM fragment made according to an in vitro synthesis method disclosed herein has a different modification profile compared to a TREM expressed and isolated from a cell, or compared to a naturally occurring tRNA.

An exemplary method for making a synthetic TREM via 5′-Silyl-2′-Orthoester (2′-ACE) Chemistry is provided in Example 3. The method provided in Example 3 can also be used to make a synthetic TREM core fragment or synthetic TREM fragment. Additional synthetic methods are disclosed in Hartsel S A et al., (2005) Oligonucleotide Synthesis, 033-050, the entire contents of which are hereby incorporated by reference.

### TREM Composition

In an embodiment, a TREM composition, e.g., a TREM pharmaceutical composition, comprises a pharmaceutically acceptable excipient. Exemplary excipients include those provided in the FDA Inactive Ingredient Database

(https://www.accessdata.fda.gov/scripts/cder/iig/index.Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM, TREM core fragment or TREM fragment. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM, TREM core fragment or TREM fragment.

In an embodiment, a TREM composition, e.g., a TREM pharmaceutical composition, is at least 10, 20, 30, 40, 50, 60, 70, 80, 90, 95 or 99% dry weight TREMs, TREM core fragments or TREM fragments.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition comprises at least 1×10⁶ TREM core fragment molecules, at least 1×10⁷ TREM core fragment molecules, at least 1×10⁸ TREM core fragment molecules or at least 1×10⁹ TREM core fragment molecules.

In an embodiment, a TREM composition comprises at least 1×10⁶ TREM fragment molecules, at least 1×10⁷ TREM fragment molecules, at least 1×10⁸ TREM fragment molecules or at least 1×10⁹ TREM fragment molecules.

In an embodiment, a TREM composition produced by any of the methods of making disclosed herein can be charged with an amino acid using an in vitro charging reaction as known in the art.

In an embodiment, a TREM composition comprise one or more species of TREMs, TREM core fragments, or TREM fragments. In an embodiment, a TREM composition comprises a single species of TREM, TREM core fragment, or TREM fragment. In an embodiment, a TREM composition comprises a first TREM, TREM core fragment, or TREM fragment species and a second TREM, TREM core fragment, or TREM fragment species. In an embodiment, the TREM composition comprises X TREM, TREM core fragment, or TREM fragment species, wherein X=2, 3, 4, 5, 6, 7, 8, 9, or 10.

In an embodiment, the TREM, TREM core fragment, or TREM fragment has at least 70, 75, 80, 85, 90, or 95, or has 100%, identity with a sequence encoded by a nucleic acid in Table 4.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, TREM core fragment, or TREM fragment.

In some embodiments, a TREM composition can be formulated with water for injection. In some embodiments, a TREM composition formulated with water for injection is suitable for pharmaceutical use, e.g., comprises a pharmaceutical TREM composition.

### TREM Characterization

A TREM, TREM core fragment, or TREM fragment, or a TREM composition, e.g., a pharmaceutical TREM composition, produced by any of the methods disclosed herein can be assessed for a characteristic associated with the TREM, TREM core fragment, or TREM fragment or the TREM composition, such as purity, sterility, concentration, structure, or functional activity of the TREM, TREM core fragment, or TREM fragment. Any of the above-mentioned characteristics can be evaluated by providing a value for the characteristic, e.g., by evaluating or testing the TREM, TREM core fragment, or TREM fragment, or the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of fragments in the composition; (iii) decrease the amount of endotoxins in the composition; (iv) increase the in vitro translation activity of the composition; (v) increase the TREM concentration of the composition; or (vi) inactivate or remove any viral contaminants present in the composition, e.g., by reducing the pH of the composition or by filtration.

In an embodiment, the TREM, TREM core fragment, or TREM fragment (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has less than 0.1%, 0.5%, 1%, 2%, 3%, 4%, 5%, 6%, 7%, 8%, 9%, 10%, 15%, 20%, 25% TREM fragments relative to full length TREMs.

In an embodiment, the TREM, TREM core fragment, or TREM fragment (e.g., TREM composition or an intermediate in the production of the TREM composition) has low levels or absence of endotoxins, e.g., a negative result as measured by the Limulus amebocyte lysate (LAL) test.

In an embodiment, the TREM, TREM core fragment, or TREM fragment (e.g., TREM composition or an intermediate in the production of the TREM composition) has in-vitro translation activity, e.g., as measured by an assay described in Examples 12-13.

In an embodiment, the TREM, TREM core fragment, or TREM fragment (e.g., TREM composition or an intermediate in the production of the TREM composition) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM, TREM core fragment, or TREM fragment (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM, TREM core fragment, or TREM fragment (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

Any TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers

In some embodiments the TREM, TREM core fragment, or TREM fragment or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, or an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM, a TREM core fragment or a TREM fragment). The viral vector may be administered to a cell or to a subject (e.g., a human subject or animal model) to deliver a TREM, a TREM core fragment or a TREM fragment, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM core fragment or a TREM fragment, a TREM composition or a pharmaceutical TREM composition described herein can be administered to a cell in a vesicle or other membrane-based carrier.

In embodiments, a TREM, a TREM core fragment or a TREM fragment, or TREM composition, or pharmaceutical TREM composition described herein is administered in or via a cell, vesicle or other membrane-based carrier. In one embodiment, the TREM, TREM core fragment, TREM fragment, or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the TREM, TREM core fragment, TREM fragment, or TREM composition or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] of PCT/US2014/053907 can be used as a carrier for the TREM, TREM core fragment, TREM fragment, or TREM composition or pharmaceutical TREM composition described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, TREM core fragment, TREM fragment, or TREM composition or pharmaceutical TREM composition described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

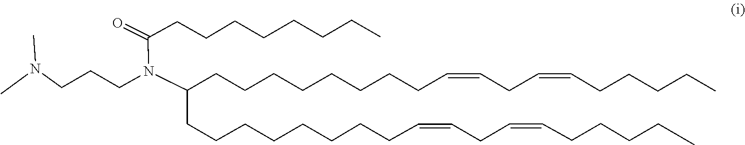 [SMILES: CCCCC/C=C\C/C=C\CCCCCCCCC(CCCCCCCC/C=C\C/C=C\CCCCCC)N(CCCN(C)C)C(=O)CCCCCCCCC]

In some embodiments an LNP comprising Formula (i) is used to deliver a TREM composition described herein to the liver and/or hepatocyte cells.

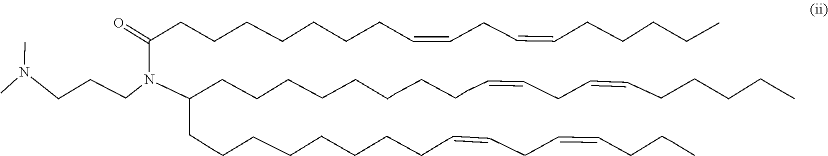 [SMILES: CCCC/C=C\C/C=C\CCCCCCCCCCC(CCCCCCCCCC/C=C\C/C=C\CCCCCC)N(CCCN(C)C)C(=O)CCCCCCC/C=C\CC/C=C\CCCCC]

In some embodiments an LNP comprising Formula (ii) is used to deliver a TREM composition described herein to the liver and/or hepatocyte cells.

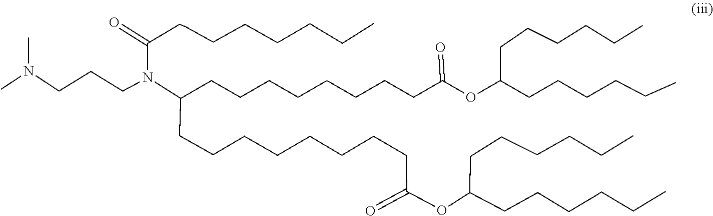 [SMILES: CCCCCCCCCC(=O)N(CCCN(C)C)C(CCCCCCCCC(=O)OC(CCCCCC)CCCCCC)CCCCCCCCC(=O)OC(CCCCCC)CCCCCCC]

In some embodiments an LNP comprising Formula (iii) is used to deliver a TREM composition described herein to the liver and/or hepatocyte cells.

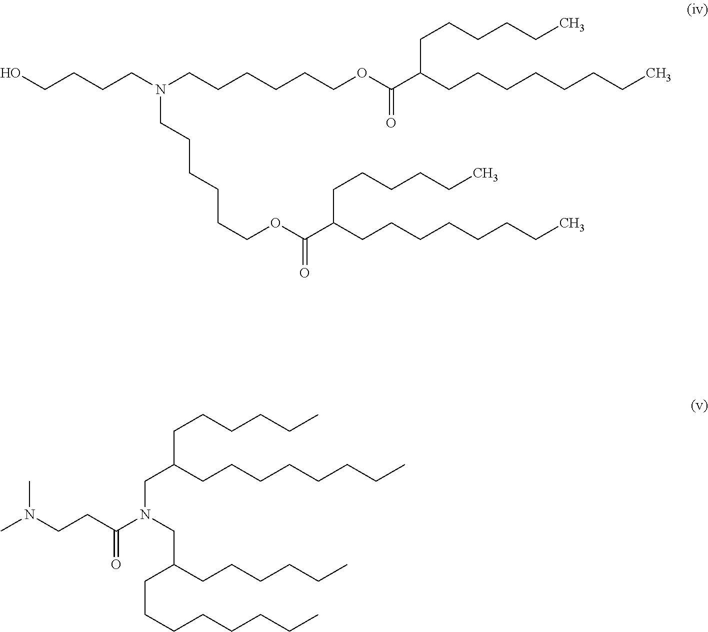

In some embodiments an LNP comprising Formula (v) is used to deliver a TREM composition described herein to the liver and/or hepatocyte cells.

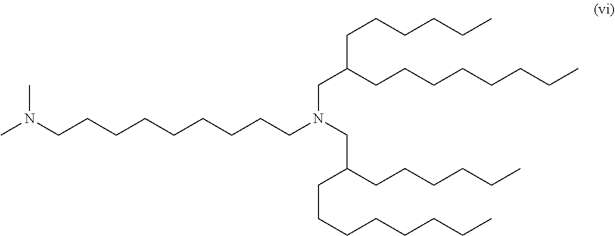 [SMILES: CCCCCCCCC(CCCCCC)CN(CCCCCCCCCN(C)C)CC(CCCCCC)CCCCCCCC]

In some embodiments an LNP comprising Formula (vi) is used to deliver a TREM composition described herein to the liver and/or hepatocyte cells.

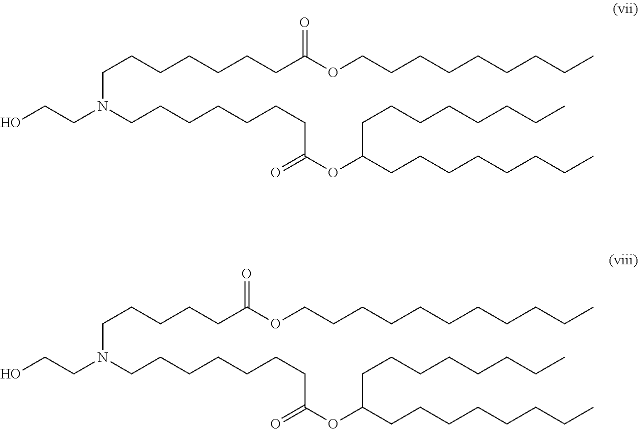 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

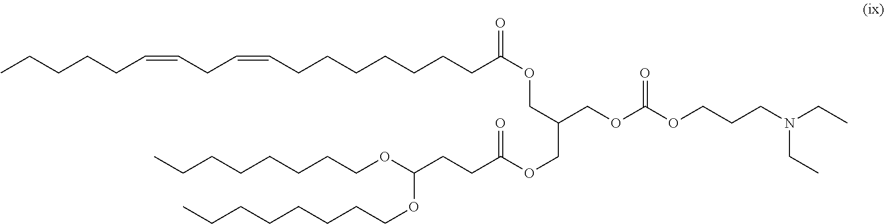 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a TREM composition described herein to the liver and/or hepatocyte cells.

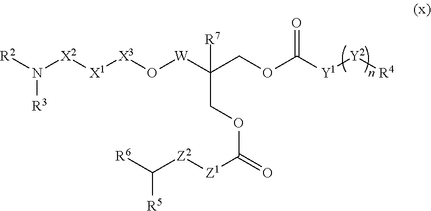

wherein X¹ is O, NR¹ or a direct bond, X² is C2-5 alkylene, X³ is C(═O) or a direct bond, R¹ is H or Me. R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached from a 5- or 6-membered ring, or R² taken together with R³ and the nitrogen atom to which they are attached form a 5-, 6-, or 7-membered ring, Y is C2-12 alkylene. Y² is selected from

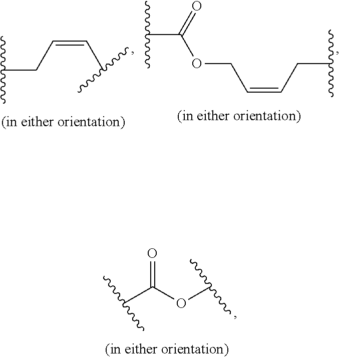 [SMILES: */C=C\C* ; *C/C=C\COC(*)=O ; *OC(*)=O]

n is 0 to 3, R⁴ is Ci-15 alkyl, Z¹ is Ci-6 alkylene or a direct bond, Z² is

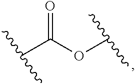 [SMILES: *OC(*)=O]

(in either orientation) or absent, provided that if Z¹ is a direct bond, Z² is absent; R² is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls. X¹ is O, X² is linear C3 alkylene, X³ is C(═O), Y¹ is linear Ce alkylene, (Y²)n-R⁴ is

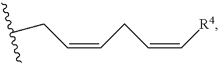

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

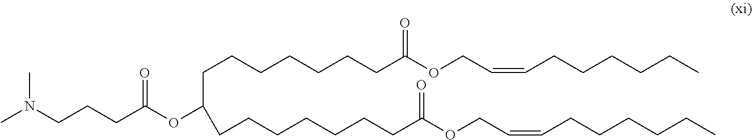 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

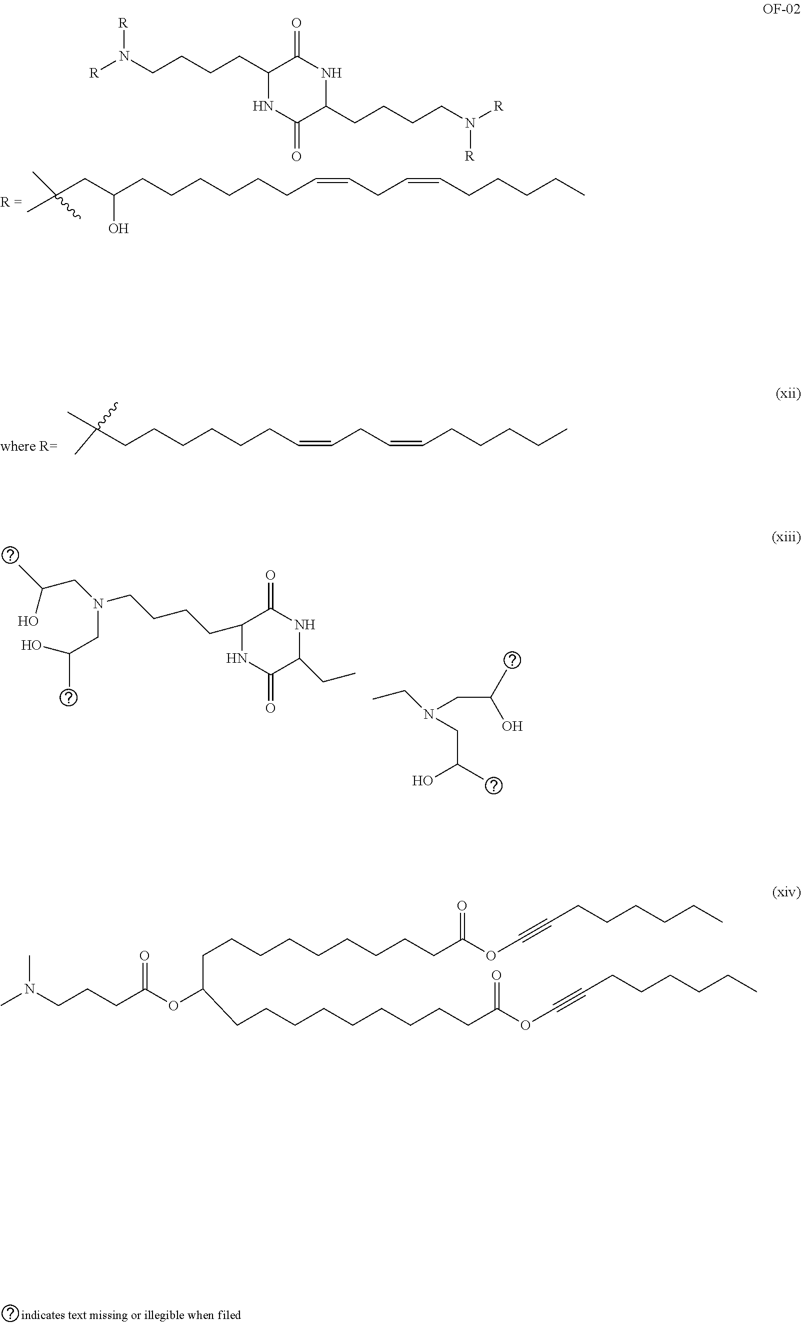

In some embodiments an LNP comprises a compound of Formula (xiii) and a compound of Formula (xiv).

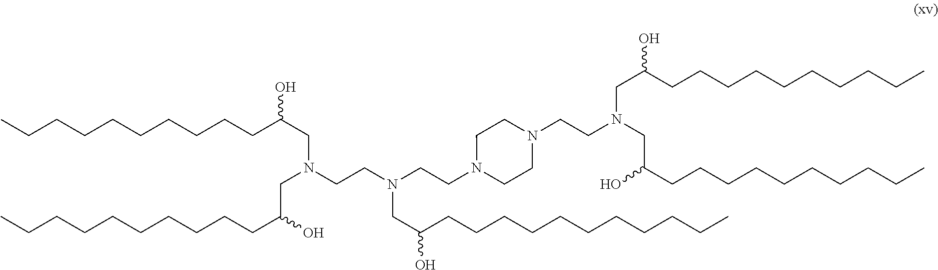 [SMILES: CCCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

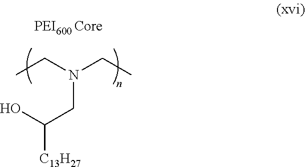

In some embodiments an LNP comprising a formulation of Formula (xvi) is used to deliver a TREM composition described herein to the lung endothelial cells.

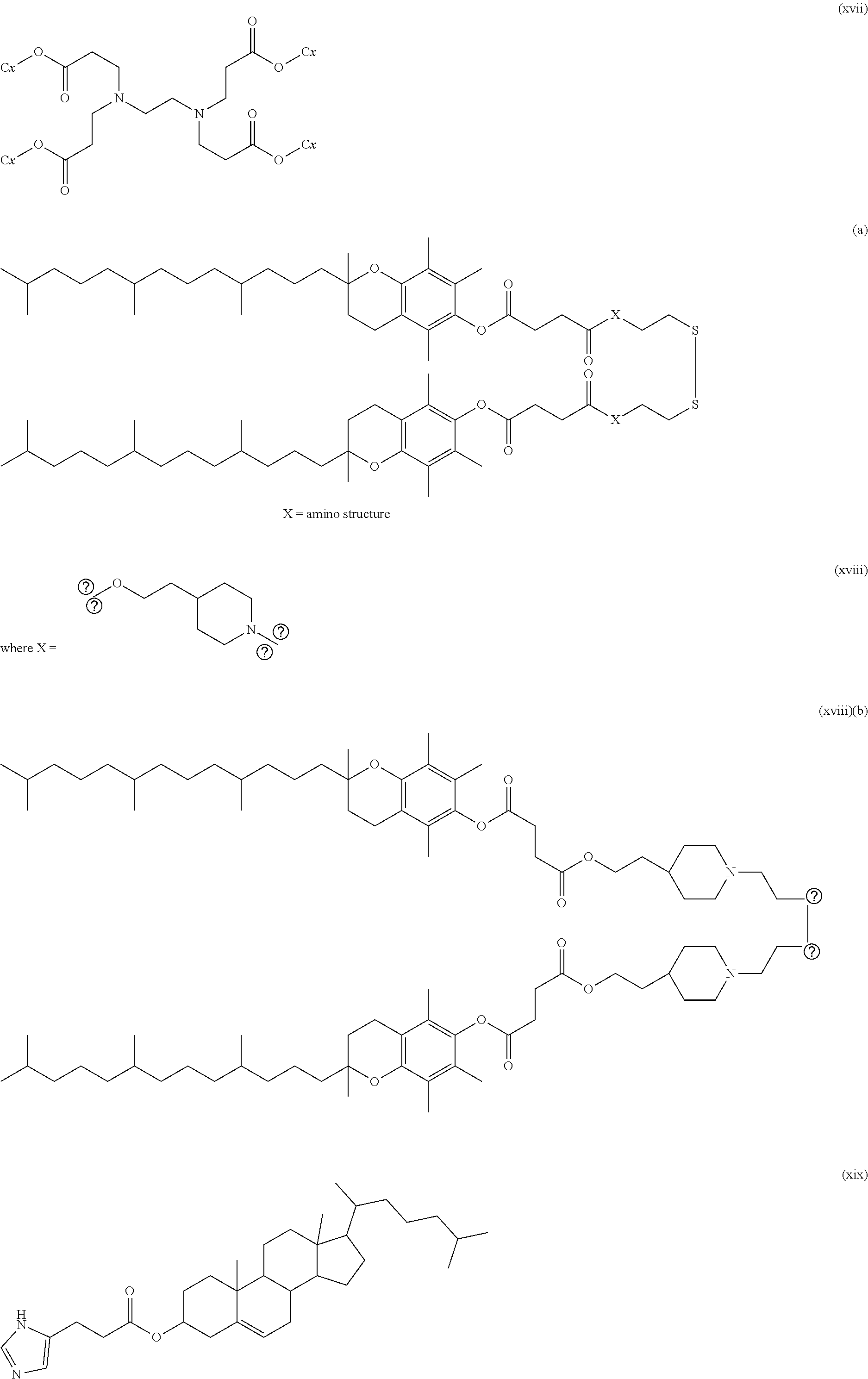

In some embodiments, a lipid compound used to form lipid nanoparticles for the delivery of compositions described herein, e.g., a TREM described herein is made by one of the following reactions:

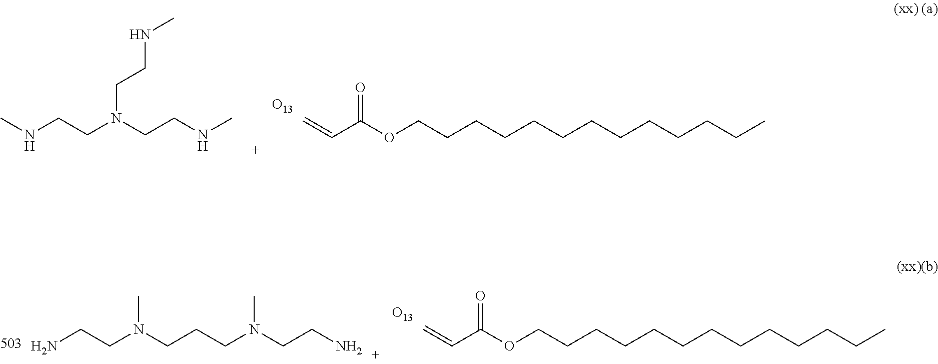

In some embodiments, a composition described herein (e.g., TREM composition) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)-butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 1Z)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.Oc01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

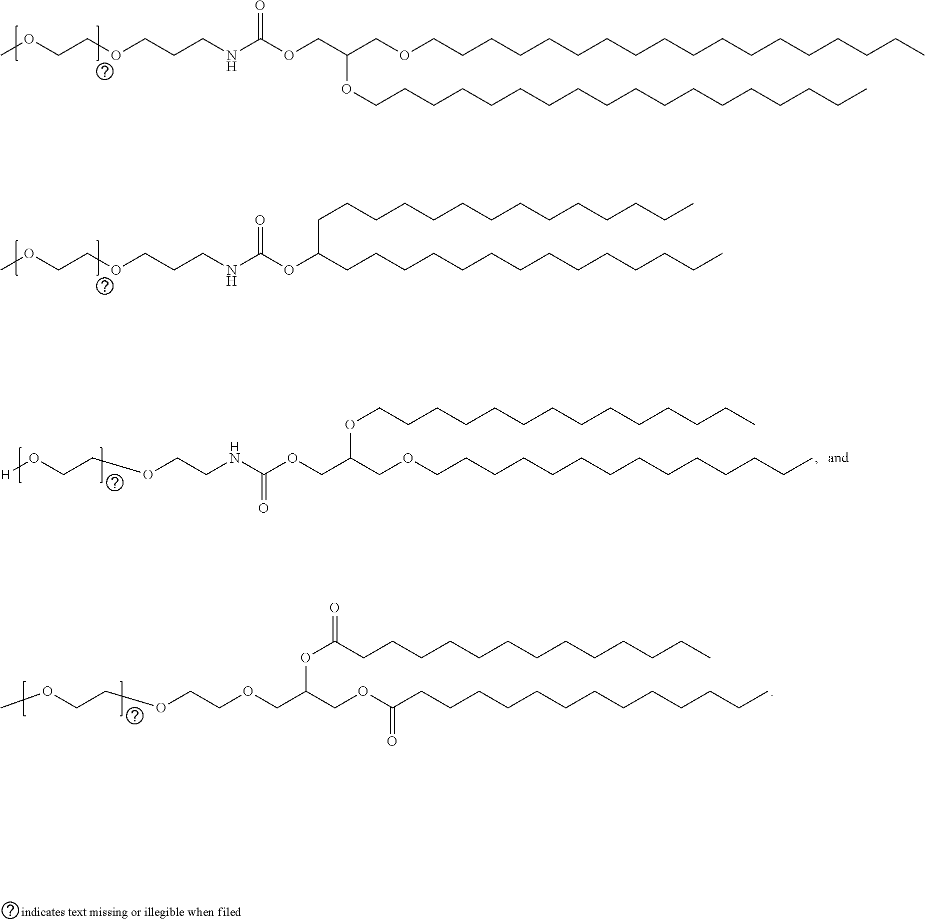

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10 s of nm and 100 s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with a LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM, TREM core fragment, TREM fragment, or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM core fragment, TREM fragment, or TREM composition, or pharmaceutical TREM composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the TREM, TREM core fragment, TREM fragment, or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

Delivery without a Carrier

A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

All references and publications cited herein are hereby incorporated by reference.

### ENUMERATED EMBODIMENTS

1. A TREM comprising a sequence of Formula A:

[L1]-[ASt Domain1]-[L2]-[DH Domain]-[L3]-[ACH Domain]-[VL Domain]-[TH Domain]-[L4]-[ASt Domain2],

wherein:

independently, [L1] and [VL Domain], are optional;

one of [L1], [ASt Domain1], [L2]-[DH Domain], [L3], [ACH Domain], [VL Domain], [TH Domain], [L4], and [ASt Domain2] comprises a nucleotide having a non-naturally occurring modification; and

wherein:
(a) the TREM retains the ability to: support protein synthesis, be charged by a synthetase, be bound by an elongation factor, introduce an amino acid into a peptide chain, support elongation, or support initiation;
(b) the TREM comprises at least X contiguous nucleotides without a non-naturally occurring modification, wherein X is greater than 10;
(c) at least 3, but less than all of the nucleotides of a type (e.g., A, T, C, G or U) comprise the same non-naturally occurring modification;
(d) at least X nucleotides of a type (e.g., A, T, C, G or U) do not comprise a non-naturally occurring modification, wherein X=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50;
(e) no more than 5, 10, or 15 nucleotides of a type (e.g., A, T, C, G or U) comprise a non-naturally occurring modification; and/or
(f) no more than 5, 10, or 15 nucleotides of a type (e.g., A, T, C, G or U) do not comprise a non-naturally occurring modification.
   2. The TREM of embodiment 1, comprising the feature provided in embodiment 1(a).
   3. The TREM of embodiment 1, comprising the feature provided in embodiment 1(b).
   4. The TREM of embodiment 1, comprising the feature provided in embodiment 1(c).
   5. The TREM of embodiment 1, comprising the feature provided in embodiment 1(d).
   6. The TREM of embodiment 1, comprising the feature provided in embodiment 1(e).
   7. The TREM of embodiment 1, comprising the feature provided in embodiment 1(f).
   8. The TREM of embodiment 1, comprising all of the features provided in embodiments 1(a)-(f).
   9. The TREM of any one of embodiments 1-8, wherein the Domain comprising the non-naturally occurring modification retains a function, e.g., a domain function described herein.
   10. The TREM of any one of embodiments 1-8, comprising an [L1].
   11. The TREM of any one of embodiments 1-8, comprising a [VL Domain].
   12. The TREM of any one of embodiments 1-8, wherein: [L1] is a linker comprising a nucleotide having a non-naturally occurring modification.
   13. The TREM of any one of embodiments 1-8, wherein [ASt Domain1 (AstD1)] comprises a nucleotide having a non-naturally occurring modification.
   14. The TREM of any one of embodiments 1-8, wherein [L2] is a linker comprising a nucleotide having a non-naturally occurring modification.
   15. The TREM of any one of embodiments 1-8, wherein [DH Domain (DHD)] comprises a nucleotide having a non-naturally occurring modification.
   16. The TREM of any one of embodiments 1-8, wherein [L3] is a linker comprising a nucleotide having a non-naturally occurring modification.
   17. The TREM of any one of embodiments 1-8, wherein [ACH Domain (ACHD)] comprises a nucleotide having a non-naturally occurring modification.
   18. The TREM of any one of embodiments 1-8, wherein [VL Domain (VLD)] comprises a nucleotide having a non-naturally occurring modification.
   19. The TREM of any one of embodiments 1-8, wherein [TH Domain (THD)] comprises a nucleotide having a non-naturally occurring modification.
   20. The TREM of any one of embodiments 1-8, wherein [L4] is a linker comprises a nucleotide having a non-naturally occurring modification.
   21. The TREM of any one of embodiments 1-8, wherein: [ASt Domain2 (AStD2)] comprises a nucleotide having a non-naturally occurring modification.
   22. A TREM core fragment comprising a sequence of Formula B:

[L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ,

wherein:

x=1 and y=0 or 1;

one of [ASt Domain1], [ACH Domain], and [ASt Domain2] comprises a nucleotide having a non-naturally occurring modification; and

the TREM retains the ability to: support protein synthesis; be able to be charged by a synthetase, be bound by an elongation factor, introduce an amino acid into a peptide chain, support elongation, or support initiation.

23. The TREM core fragment of embodiment 22, wherein AStD1 and AStD2 comprise an ASt Domain (AStD).
24. The TREM core fragment of embodiment 22, wherein the [ASt Domain 1], and/or [ASt Domain 2] comprising the non-naturally occurring modification retains the ability to initiate or elongate a polypeptide chain.
25. The TREM core fragment of embodiment 22, wherein the [ACH Domain] comprising the non-naturally occurring modification retains the ability to mediate pairing with a codon.
26. The TREM core fragment of embodiment 22, wherein y=1 for any one, two, three, four, five, six, all or a combination of [L1], [L2], [DH Domain], [L3], [VL Domain], [TH Domain], [L4].
27. The TREM core fragment of embodiment 22, wherein y=0 for any one, two, three, four, five, six, all or a combination of [L1], [L2], [DH Domain], [L3], [VL Domain], [TH Domain], [L4].
28. The TREM core fragment of embodiment 22, wherein y=1 for linker [L1], and L1 comprises a nucleotide having a non-naturally occurring modification.
29. The TREM core fragment of embodiment 22, wherein y=1 for linker [L2], and L2 comprises a nucleotide having a non-naturally occurring modification.
30. The TREM core fragment of embodiment 22, wherein y=1 for [DH Domain (DHD)], and DHD comprises a nucleotide having a non-naturally occurring modification.
31. The TREM core fragment of embodiment 30, wherein the DHD comprising the non-naturally occurring modification retains the ability to mediate recognition of aminoacyl-tRNA synthetase.
32. The TREM core fragment of embodiment 22, wherein y=1 for linker [L3], and L3 comprises a nucleotide having a non-naturally occurring modification.
33. The TREM core fragment of embodiment 22, wherein y=1 for [VL Domain (VLD)], and VLD comprises a nucleotide having a non-naturally occurring modification.
34. The TREM core fragment of embodiment 22, wherein y=1 for [TH Domain (THD)], and THD comprises a nucleotide having a non-naturally occurring modification.
35. The TREM core fragment of embodiment 34, wherein the THD comprising the non-naturally occurring modification retains the ability to mediate recognition of the ribosome.
36. The TREM core fragment of embodiment 22, wherein y=1 for linker [L4], and L4 comprises a nucleotide having a non-naturally occurring modification.
37. A TREM fragment comprising a portion of a TREM, wherein the TREM comprises a sequence of Formula A:

[L1]-[ASt Domain1]-[L2]-[DH Domain]-[L3]-[ACH Domain]-[VL Domain]-[TH Domain]-[L4]-[ASt Domain2], and wherein:

the TREM fragment comprises:

a non-naturally occurring modification; and

one, two, three or all or any combination of the following:
(a) a TREM half (e.g., from a cleavage in the ACH Domain, e.g., in the anticodon sequence, e.g., a 5′ half or a 3′ half);
(b) a 5′ fragment (e.g., a fragment comprising the 5′ end, e.g., from a cleavage in a DH Domain or the ACH Domain);
(c) a 3′ fragment (e.g., a fragment comprising the 3′ end, e.g., from a cleavage in the TH Domain); or
(d) an internal fragment (e.g., from a cleavage in any one of the ACH Domain, DH Domain or TH Domain).
   38. The TREM of embodiment 37, wherein the TREM fragment comprise (a) a TREM half which comprises a nucleotide having a non-naturally occurring modification.
   39. The TREM of embodiment 37, wherein the TREM fragment comprise (b) a 5′ fragment which comprises a nucleotide having a non-naturally occurring modification.
   40. The TREM of embodiment 37, wherein the TREM fragment comprise (c) a 3′ fragment which comprises a nucleotide having a non-naturally occurring modification.
   41. The TREM of embodiment 37, wherein the TREM fragment comprise (d) an internal fragment which comprises a nucleotide having a non-naturally occurring modification.
   42. The TREM of any one of embodiments 1-8, the TREM core fragment of embodiment 22, or the TREM fragment of embodiment 37, wherein the TREM Domain comprises a plurality of nucleotides each having a non-naturally occurring modification.
   43. The TREM of any one of embodiments 1-8, the TREM core fragment of embodiment 22, or the TREM fragment of embodiment 37, wherein at least X of the nucleotides of AStD1 have a non-naturally occurring modification, wherein X is equal to or greater than 1, 2, 3, 4, 5, 6 or 7.
   44. The TREM of any one of embodiments 1-8, the TREM core fragment of embodiment 22, or the TREM fragment of embodiment 37, wherein no more than X of the nucleotides of AStD1 have a non-naturally occurring modification, wherein X is equal to or greater than 1, 2, 3, 4, 5, 6 or 7.
   45. The TREM of any one of embodiments 1-8, the TREM core fragment of embodiment 22, or the TREM fragment of embodiment 37, wherein at least X of the nucleotides of AStD2 have a non-naturally occurring modification, wherein X is equal to or greater than 1, 2, 3, 4, 5, 6 or 7.
   46. The TREM of any one of embodiments 1-8, the TREM core fragment of embodiment 22, or the TREM fragment of embodiment 37, wherein no more than X of the nucleotides of AStD2 have a non-naturally occurring modification, wherein X is equal to or greater than 1, 2, 3, 4, 5, 6 or 7.
   47. The TREM of any one of embodiments 1-8, the TREM core fragment of embodiment 22, or the TREM fragment of embodiment 37, wherein at least X of the nucleotides of ACHD have a non-naturally occurring modification, wherein X is equal to or greater than 1, 2, 3, 4, 5, 6, 7, 8, 9, or 10.
   48. The TREM of any one of embodiments 1-8, the TREM core fragment of embodiment 22, or the TREM fragment of embodiment 37, wherein at least X of the nucleotides of ACHD have a non-naturally occurring modification, wherein X is equal to or greater than 11, 12, 13, 14, 15, 16, or 17.
   49. The TREM of any one of embodiments 1-8, the TREM core fragment of embodiment 22, or the TREM fragment of embodiment 37, wherein no more than X of the nucleotides of ACHD have a non-naturally occurring modification, wherein X is equal to or greater than 1, 2, 3, 4, 5, 6, 7, 8, 9, or 10.
   50. The TREM of any one of embodiments 1-8, the TREM core fragment of embodiment 22, or the TREM fragment of embodiment 37, wherein no more than X of the nucleotides of ACHD have a non-naturally occurring modification, wherein X is equal to or greater than 11, 12, 13, 14, 15, or 16.
   51. The TREM of any one of embodiments 1-8, the TREM core fragment of embodiment 22, or the TREM fragment of embodiment 37, wherein at least X of the nucleotides of THD have a non-naturally occurring modification, wherein X is equal to or greater than 1, 2, 3, 4, 5, 6, 7, 8, 9, or 10.
   52. The TREM of any one of embodiments 1-8, the TREM core fragment of embodiment 22, or the TREM fragment of embodiment 37, wherein at least X of the nucleotides of THD have a non-naturally occurring modification, wherein X is equal to or greater than 11, 12, 13, 14, 15, 16, or 17.
   53. The TREM of any one of embodiments 1-8, the TREM core fragment of embodiment 22, or the TREM fragment of embodiment 37, wherein no more than X of the nucleotides of THD have a non-naturally occurring modification, wherein X is equal to or greater than 1, 2, 3, 4, 5, 6, 7, 8, 9, or 10.
   54. The TREM of any one of embodiments 1-8, the TREM core fragment of embodiment 22, or the TREM fragment of embodiment 37, wherein no more than X of the nucleotides of THD have a non-naturally occurring modification, wherein X is equal to or greater than 11, 12, 13, 14, 15, or 16.
   55. The TREM of any one of embodiments 1-8, the TREM core fragment of embodiment 22, or the TREM fragment of embodiment 37, wherein at least X of the nucleotides of DHD have a non-naturally occurring modification, wherein X is equal to or greater than 2, 3, 4, 5, 6, 7, 8, 9 or 10.
   56. The TREM of any one of embodiments 1-8, the TREM core fragment of embodiment 22, or the TREM fragment of embodiment 37, wherein at least X of the nucleotides of DHD have a non-naturally occurring modification, wherein X is equal to or greater than 11, 12, 13, 14, 15, 16, 17, 18 or 19.
   57. The TREM of any one of embodiments 1-8, the TREM core fragment of embodiment 22, or the TREM fragment of embodiment 37, wherein no more than X of the nucleotides of DHD have a non-naturally occurring modification, wherein X is equal to or greater than 1, 2, 3, 4, 5, 6, 7, 8, 9, or 10.
   58. The TREM of any one of embodiments 1-8, the TREM core fragment of embodiment 22, or the TREM fragment of embodiment 37, wherein no more than X of the nucleotides of DHD have a non-naturally occurring modification, wherein X is equal to or greater than 11, 12, 13, 14, 15, 16, 17, or 18.
   59. The TREM of any one of embodiments 1-8, the TREM core fragment of embodiment 22, or the TREM fragment of embodiment 37, wherein at least X of the nucleotides of the VLD have a non-naturally occurring modification, wherein X is equal to or greater than 2, 3, 4, 5, 6, 7, 8, 9, 10, 11, 12, 13, 14, 15, 16, 17, 18, 19, 20, 50, 100, 150, 200 or 271.
   60. The TREM of any one of embodiments 1-8, the TREM core fragment of embodiment 22, or the TREM fragment of embodiment 37, wherein all of the nucleotides of the AStD1, AStD2, ACHD, DHD, and/or THD have a non-naturally occurring modification.
   61. The TREM of any one of embodiments 1-8, the TREM core fragment of embodiment 22, or the TREM fragment of embodiment 37, wherein at least X of the nucleotides of AStD1 and/or AStD2 do not have a non-naturally occurring modification, wherein X is equal to or greater than 1, 2, 3, 4, 5, 6 or 7.
   62. The TREM of any one of embodiments 1-8, the TREM core fragment of embodiment 22, or the TREM fragment of embodiment 37, wherein at least X of the nucleotides of ACHD do not have a non-naturally occurring modification, wherein X is equal to or greater than 1, 2, 3, 4, 5, 6, 7, 8, 9, 10, 11, 12, 13, 14, 15, 16, or 17.
   63. The TREM of any one of embodiments 1-8, the TREM core fragment of embodiment 22, or the TREM fragment of embodiment 37, wherein at least X of the nucleotides of THD do not have a non-naturally occurring modification, wherein X is equal to or greater than 1, 2, 3, 4, 5, 6, 7, 8, 9, 10, 11, 12, 13, 14, 15, 16, or 17.
   64. The TREM of any one of embodiments 1-8, the TREM core fragment of embodiment 22, or the TREM fragment of embodiment 37, wherein at least X of the nucleotides of DHD do not have a non-naturally occurring modification, wherein X is equal to or greater than 1, 2, 3, 4, 5, 6, 7, 8, 9, 10, 11, 12, 13, 14, 15, 16, 17, 18 or 19.
   65. The TREM of any one of embodiments 1-8, the TREM core fragment of embodiment 22, or the TREM fragment of embodiment 37, wherein at least X of the nucleotides of VLD do not have a non-naturally occurring modification, wherein X is equal to or greater than 1, 2, 3, 4, 5, 6, 7, 8, 9, 10, 11, 12, 13, 14, 15, 16, 17, 18, 19, 20, 50, 100, 150, 200 or 271.
   66. The TREM of any one of embodiments 1-8, the TREM core fragment of embodiment 22, or the TREM fragment of embodiment 37, wherein the TREM Linker L2 comprises two nucleotides each having a non-naturally occurring modification.
   67. The TREM of any one of embodiments 1-8, the TREM core fragment of embodiment 22, or the TREM fragment of embodiment 37, wherein at least X of the nucleotides of the TREM Linker do not have a non-naturally occurring modification, wherein X is equal to 1 or 2.
   68. The TREM of any one of embodiments 1-8, the TREM core fragment of embodiment 22, or the TREM fragment of embodiment 37, wherein: each of a plurality of TREM Domains and Linkers comprises a nucleotide having a non-naturally occurring modification.
   69. The TREM, TREM core fragment or TREM fragment of embodiment 68, wherein one of the TREM Domains and Linkers of the plurality comprises a plurality of nucleotides each having a non-naturally occurring modification.
   70. The TREM, TREM core fragment or TREM fragment of any of the preceding embodiments, wherein the non-naturally occurring modification is a modification in a base or a backbone of a nucleotide, e.g., a modification chosen from any one of Tables 5-9.
   71. The TREM, TREM core fragment or TREM fragment of any of the preceding embodiments, wherein the non-naturally occurring modification is a base modification chosen from a modification listed in Table 5.
   72. The TREM, TREM core fragment or TREM fragment of any of the preceding embodiments, wherein the non-naturally occurring modification is a base modification chosen from a modification listed in Table 6.
   73. The TREM, TREM core fragment or TREM fragment of any of the preceding embodiments, wherein the non-naturally occurring modification is a base modification chosen from a modification listed in Table 7.
   74. The TREM, TREM core fragment or TREM fragment of any of the preceding embodiments, wherein the non-naturally occurring modification is a backbone base modification chosen from a modification listed in Table 8.
   75. The TREM, TREM core fragment or TREM fragment of any of the preceding embodiments, wherein the non-naturally occurring modification is a backbone modification chosen from a modification listed in Table 9.
   76. The TREM of any one of embodiments 1-8, the TREM core fragment of embodiment 22, or the TREM fragment of embodiment 37, comprising a nucleotide of a first type comprising a non-naturally occurring modification.
   77. The TREM of any one of embodiments 1-8, the TREM core fragment of embodiment 22, or the TREM fragment of embodiment 37, comprising a nucleotide of a first type and a nucleotide of a second type comprising a non-naturally occurring modification.
   78. The TREM, TREM core fragment or TREM fragment of embodiment 77, wherein the non-naturally occurring modification on the nucleotide of the first type and the non-naturally occurring modification on the nucleotide of the second type are the same non-naturally occurring modification.
   79. The TREM, TREM core fragment or TREM fragment of embodiment 77, wherein the non-naturally occurring modification on the nucleotide of the first type and the non-naturally occurring modification on the nucleotide of the second type are different non-naturally occurring modifications.
   80. The TREM, TREM core fragment or TREM fragment of embodiments 76 or 77, wherein the nucleotide of the first type is chosen from: A, T, C, G or U.
   81. The TREM, TREM core fragment or TREM fragment of embodiments 76 or 77, wherein the nucleotide of the second type is chosen from: A, T, C, G or U.
   82. The TREM, TREM core fragment or TREM fragment of embodiments 76 or 77, wherein the nucleotide of the first type is an A.
   83. The TREM, TREM core fragment or TREM fragment of embodiments 76 or 77, wherein the nucleotide of the first type is a G.
   84. The TREM, TREM core fragment or TREM fragment of embodiments 76 or 77, wherein the nucleotide of the first type is a C.
   85. The TREM core fragment or TREM fragment of embodiments 76 or 77, wherein the nucleotide of the first type is a T.
   86. The TREM, TREM core fragment or TREM fragment of embodiments 76 or 77, wherein the nucleotide of the first type is a U.
   87. The TREM, TREM core fragment or TREM fragment of embodiment 77, wherein when the nucleotide of the first type is an A, the nucleotide of the second type is chosen from: T, C, G or U.
   88. The TREM, TREM core fragment or TREM fragment of embodiment 77, wherein when the nucleotide of the first type is a G, the nucleotide of the second type is chosen from: T, C, A or U.
   89. The TREM, TREM core fragment or TREM fragment of embodiment 77, wherein when the nucleotide of the first type is a C, the nucleotide of the second type is chosen from: T, A, G or U.
   90. The TREM, TREM core fragment or TREM fragment of embodiment 77, wherein when the nucleotide of the first type is a T, the nucleotide of the second type is chosen from: A, C, G or U.
   91. The TREM, TREM core fragment or TREM fragment of embodiment 77, wherein when the nucleotide of the first type is a U, the nucleotide of the second type is chosen from: T, C, G or A.
   92. The TREM of any one of embodiments 1-8, the TREM core fragment of embodiment 22, or the TREM fragment of embodiment 37, wherein the non-naturally modification is in a purine (A or G).
   93. The TREM of any one of embodiments 1-8, the TREM core fragment of embodiment 22, or the TREM fragment of embodiment 37, wherein the non-naturally modification is not in a purine (A or G).
   94. The TREM of any one of embodiments 1-8, the TREM core fragment of embodiment 22, or the TREM fragment of embodiment 37, wherein the non-naturally modification is in a pyrimidine (U, T or C).
   95. The TREM of any one of embodiments 1-8, the TREM core fragment of embodiment 22, or the TREM fragment of embodiment 37, wherein the non-naturally modification is not in a pyrimidine (U, T or C).
   96. The TREM of any one of embodiments 1-8, the TREM core fragment of embodiment 22, or the TREM fragment of embodiment 37, wherein the DHD has a first sequence, a second sequence and a third sequence, optionally wherein the first sequence and the third sequence form a stem and the second sequence forms a loop, e.g., under physiological conditions.
   97. The TREM, TREM core fragment or TREM fragment of embodiment 96, wherein the DHD comprises a non-naturally occurring modification in the first sequence or the third sequence, e.g., in the stem.
   98. The TREM, TREM core fragment or TREM fragment of embodiment 96, wherein the DHD comprises a non-naturally occurring modification in the second sequence, e.g., in the loop.
   100. The TREM of any one of embodiments 1-8, the TREM core fragment of embodiment 22, or the TREM fragment of embodiment 37, wherein the ACHD has a first sequence, a second sequence and a third sequence, optionally wherein the first sequence and the third sequence form a stem and the second sequence forms a loop, e.g., under physiological conditions.
   101. The TREM, TREM core fragment or TREM fragment of embodiment 100, wherein the ACHD comprises a non-naturally occurring modification in the first sequence or the third sequence, e.g., in the stem.
   102. The TREM, TREM core fragment or TREM fragment of embodiment 100, wherein the ACHD comprises a non-naturally occurring modification in the second sequence, e.g., in the loop.
   103. The TREM of any one of embodiments 1-8, the TREM core fragment of embodiment 22, or the TREM fragment of embodiment 37, wherein the THD has a first sequence, a second sequence and a third sequence, optionally wherein the first sequence and the third sequence form a stem and the second sequence forms a loop, e.g., under physiological conditions.
   104. The TREM, TREM core fragment or TREM fragment of embodiment 103, wherein the THD comprises a non-naturally occurring modification in the first sequence or the third sequence, e.g., in the stem.
   105. The TREM, TREM core fragment or TREM fragment of embodiment 103, wherein the THD comprises a non-naturally occurring modification in the second sequence, e.g., in the loop.
   106. The TREM of any one of embodiments 1-8, the TREM core fragment of embodiment 22, or the TREM fragment of embodiment 37, wherein the VLD comprises a variable region having 1-271 nucleotides.
   107. The TREM of any one of embodiments 1-8, the TREM core fragment of embodiment 22, or the TREM fragment of embodiment 37, wherein the TREM comprises at least X contiguous nucleotides without a non-naturally occurring modification, wherein X is greater than 10.
   108. The TREM of any one of embodiments 1-8, the TREM core fragment of embodiment 22, or the TREM fragment of embodiment 37, wherein at least 3, but less than all of the nucleotides of a type (e.g., A, T, C, G or U) comprise the same non-naturally occurring modification.
   109. The TREM of any one of embodiments 1-8, the TREM core fragment of embodiment 22, or the TREM fragment of embodiment 37, wherein at least X nucleotides of a type (e.g., A, T, C, G or U) do not comprise a non-naturally occurring modification, wherein X=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50.
   110. The TREM of any one of embodiments 1-8, the TREM core fragment of embodiment 22, or the TREM fragment of embodiment 37, wherein no more than 5, 10, or 15 of a type (e.g., A, T, C, G or U) comprise a non-naturally occurring modification.
   111. The TREM of any one of embodiments 1-8, the TREM core fragment of embodiment 22, or the TREM fragment of embodiment 37, wherein no more than 5, 10, or 15 of a type (e.g., A, T, C, G or U) do not comprise a non-naturally occurring modification.
   112. The TREM of any one of embodiments 1-8, the TREM core fragment of embodiment 22, or the TREM fragment of embodiment 37, which specifies X, wherein X is an amino acid selected from alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.
   113. The TREM of any one of embodiments 1-8, the TREM core fragment of embodiment 22, or the TREM fragment of embodiment 37, which recognizes a codon provided in Table 2 or Table 3.
   114. The TREM of any one of embodiments 1-8, the TREM core fragment of embodiment 22, or the TREM fragment of embodiment 37, wherein the TREM is a cognate TREM.
   115. The TREM of any one of embodiments 1-8, the TREM core fragment of embodiment 22, or the TREM fragment of embodiment 37, wherein the TREM is a non-cognate TREM.
   116. The TREM of any one of embodiments 1-8, the TREM core fragment of embodiment 22, or the TREM fragment of embodiment 37, wherein the TREM, TREM core fragment, or TREM fragment is encoded by a sequence provided in Table 4, e.g., any one of SEQ ID NOs 1-451.
   117. The TREM of any one of embodiments 1-8, the TREM core fragment of embodiment 22, or the TREM fragment of embodiment 37, wherein the TREM, TREM core fragment, or TREM fragment is encoded by a consensus sequence chosen from any one of SEQ ID NOs: 562-621.
   118. A pharmaceutical composition comprising a TREM of any one of embodiments 1-8, the TREM core fragment of embodiment 22, or the TREM fragment of embodiment 37.
   119. The pharmaceutical composition of embodiment 118, comprising a pharmaceutically acceptable component, e.g., an excipient.
   120. A method of making a TREM of any one of embodiments 1-8, the TREM core fragment of embodiment 22, or the TREM fragment of embodiment 37, comprising linking a first nucleotide to a second nucleotide to form the TREM.
   121. The method of embodiment 120, wherein the TREM, TREM core fragment or TREM fragment is synthetic.
   122. The method of embodiment 120 or 121, wherein the synthesis is performed in vitro.
   123. The method of embodiment 120, wherein the TREM, TREM core fragment or TREM fragment is made by cell-free solid phase synthesis.
   124. A cell comprising a TREM of any one of embodiments 1-8, the TREM core fragment of embodiment 22, or the TREM fragment of embodiment 37.
   125. A cell comprising a TREM, TREM core fragment or TREM fragment made according to the method of embodiment 120.
   126. A method of evaluating a tRNA pool in a cell comprising an endogenous open reading frame (ORF), which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the cell wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the cell.
   127. A method of evaluating a tRNA pool in a subject comprising an endogenous open reading frame (ORF), which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the subject wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the subject.
   128. The method of embodiment 126 or 127, comprising acquiring knowledge of (i).
   129. The method of embodiment 126 or 127, comprising acquiring knowledge of (ii).
   130. The method of embodiment 126 or 127, comprising acquiring knowledge of (i) and (ii).
   131. The method of any one of embodiments 126-130, wherein:

acquiring knowledge of (i) comprises acquiring a value for the abundance, e.g., relative amount, of (i); and/or

acquiring knowledge of (ii) comprises acquiring a value for the abundance, e.g., relative amount, of (ii).

132. The method of embodiment 131, wherein responsive to said value, the method comprises administering a TREM composition comprising a TREM of any one of embodiments 1-8, the TREM core fragment of embodiment 22, or the TREM fragment of embodiment 37, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety.
133. A method of modulating a tRNA pool in a cell comprising an endogenous open reading frame (ORF), which ORF comprises a codon having a first sequence, comprising:

optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;

contacting the cell with a TREM composition comprising a TREM of any one of embodiments 1-8, the TREM core fragment of embodiment 22, or the TREM fragment of embodiment 37, wherein the TREM, TREM core fragment or TREM fragment has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the cell, thereby modulating the tRNA pool in the cell.

134. The method of embodiment 133, wherein the TREM comprises an anticodon that pairs with (a).
135. The method of embodiment 134, wherein the TREM comprises an anticodon that pairs with (b).
136. A method of modulating a tRNA pool in a subject having an endogenous open reading frame (ORF), which ORF comprises a codon having a first sequence, comprising:

optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;

contacting the subject with a TREM composition comprising a TREM of any one of embodiments 1-8, the TREM core fragment of embodiment 22, or the TREM fragment of embodiment 37, wherein the TREM, TREM core fragment or TREM fragment has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the subject,

thereby modulating the tRNA pool in the subject.

137. The method of embodiment 136, wherein the TREM comprises an anticodon that pairs with (a).
138 The method of embodiment 136, wherein the TREM comprises an anticodon that pairs with (b).
139. The method of any one of embodiments 136-138, comprising acquiring knowledge of (i).
140. The method of any one of embodiments 136-138, comprising acquiring knowledge of (ii).
141. The method of any one of embodiments 136-138, comprising acquiring knowledge of (i) and (ii).
142. The method of any one of embodiments 136-139 or 141, wherein acquiring knowledge of (i) comprises acquiring a value for the abundance, e.g., relative amounts, of (i).
143. The method of any one of embodiments 136-138 or 140-141, wherein acquiring knowledge of (ii) comprises acquiring a value for the abundance, e.g., relative amounts, of (ii).
144. The method of embodiment 18 or 19, wherein responsive to said value, the cell or subject is contacted with the TREM composition having an anticodon that pairs with (a) or (b).
145. A method of modulating a tRNA pool in a subject having an endogenous open reading frame (ORF) comprising a codon comprising a synonymous mutation (a synonymous mutation codon or SMC), comprising:

providing a TREM composition comprising a TREM of any one of embodiments 1-8, the TREM core fragment of embodiment 22, or the TREM fragment of embodiment 37, wherein the TREM, TREM core fragment or TREM fragment comprises an isoacceptor tRNA moiety comprising an anticodon sequence that pairs with the SMC (the TREM);

contacting the subject with the TREM composition in an amount and/or for a time sufficient to modulate the tRNA pool in the subject,

thereby modulating the tRNA pool in the subject.

146. A method of modulating a tRNA pool in a cell comprising an endogenous open reading frame (ORF) comprising a codon comprising a synonymous mutation (a synonymous mutation codon or SMC), comprising:

providing a TREM composition comprising a TREM of any one of embodiments 1-8, the TREM core fragment of embodiment 22, or the TREM fragment of embodiment 37, wherein the TREM, TREM core fragment or TREM fragment comprises an isoacceptor tRNA moiety comprising an anticodon sequence that pairs with the SMC (the TREM);

contacting the cell with the TREM composition in an amount and/or for a time sufficient to modulate the tRNA pool in the cell,

thereby modulating the tRNA pool in the cell.

147. The method of embodiment 145 or 146, comprising acquiring knowledge of the abundance of one or both of (i) and (ii) e.g., acquiring knowledge of the relative amounts of (i) and (ii) wherein (i) is a tRNA moiety having an anticodon that pairs with the SMC (the first tRNA moiety) and (ii) is an isoacceptor tRNA moiety having an anticodon that pairs with a codon other than the SMC (the second tRNA moiety), in the subject or cell.
148. The method of embodiment 147, comprising acquiring knowledge of (i).
149. The method of embodiment 147, comprising acquiring knowledge of (ii).
150. The method of embodiment 147, comprising acquiring knowledge of (i) and (ii).
151. The method of embodiment 147, wherein acquiring knowledge of (i) comprises acquiring a value for the abundance, e.g., relative amounts, of (i).
152. The method of embodiment 147, wherein acquiring knowledge of (ii) comprises acquiring a value for the abundance, e.g., relative amounts, of (ii).
153. The method of embodiment 151 or 152, wherein responsive to said value, the cell or subject is contacted with the TREM composition.
154. A method of treating a subject having an endogenous open reading frame (ORF) which comprises a codon having a first sequence, comprising:

providing a TREM composition comprising a TREM of any one of embodiments 1-8, the TREM core fragment of embodiment 22, or the TREM fragment of embodiment 37, wherein the TREM, TREM core fragment or TREM fragment comprises an isoacceptor tRNA moiety having: an anticodon that pairs with the codon of the ORF having the first sequence; or an anticodon that pairs with a codon other than the codon having the first sequence,

contacting the subject with the TREM composition in an amount and/or for a time sufficient to treat the subject,

thereby treating the subject.

155. A method of treating a subject having an endogenous open reading frame (ORF) comprising a codon comprising a synonymous mutation (a synonymous mutation codon or SMC), comprising:
providing a TREM composition comprising a TREM of any one of embodiments 1-8, the TREM core fragment of embodiment 22, or the TREM fragment of embodiment 37, wherein the TREM, TREM core fragment or TREM fragment comprises an isoacceptor tRNA moiety having an anticodon that pairs with the SMC (the TREM);

contacting the subject with the TREM composition in an amount and/or for a time sufficient to treat the subject,

thereby treating the subject.

156. The method of embodiment 154 or 155, comprising acquiring knowledge of the abundance of one or both (i) and (ii), e.g., acquiring knowledge of the relative amounts of:

(i) a tRNA moiety having an anticodon that pairs with the codon having a first sequence or the SMC (the first tRNA moiety); and/or

(ii) an isoacceptor tRNA moiety having an anticodon that pairs with a codon other than a codon having the first sequence, or pairs with a codon other than the SMC (the second tRNA moiety).

157. The method of embodiment 156, comprising acquiring knowledge of (i).
158. The method of embodiment 156, comprising acquiring knowledge of (ii).
159. The method of embodiment 156, comprising acquiring knowledge of (i) and (ii).
160. The method of embodiment 156, wherein acquiring knowledge of (i) comprises acquiring a value for the abundance, e.g., relative amounts, of (i).
161. The method of embodiment 156, wherein acquiring knowledge of (ii) comprises acquiring a value for the abundance, e.g., relative amounts, of (ii).
162. The method of embodiment 160 or 161, wherein responsive to said value, the subject is contacted with the TREM composition.
163. A method of treating a subject having an endogenous open reading frame (ORF) comprising a codon having a first sequence, comprising:

(i) acquiring, e.g., directly or indirectly acquiring, a value for the status of the codon having the first sequence in the subject, wherein said value comprises a measure of the presence or absence of the codon having the first sequence in a sample from the subject; and identifying the subject as having the codon having the first sequence; and

(ii) responsive to said value, administering a TREM composition comprising a TREM of any one of embodiments 1-8, the TREM core fragment of embodiment 22, or the TREM fragment of embodiment 37, wherein the TREM, TREM core fragment or TREM fragment comprises an isoacceptor tRNA moiety having an anticodon that pairs with the codon having the first sequence, to the subject,

thereby treating the subject.

164. A method of treating a subject having an endogenous open reading frame (ORF) comprising a codon comprising a synonymous mutation (a synonymous mutation codon or SMC), comprising:

(i) acquiring, e.g., directly or indirectly acquiring, a value for the SMC status of the subject, wherein said value comprises a measure of the presence or absence of SMC in a sample from the subject, and identifying the subject as having a SMC; and

(ii) responsive to said value, administering a TREM composition comprising a TREM of any one of embodiments 1-8, the TREM core fragment of embodiment 22, or the TREM fragment of embodiment 37, wherein the TREM, TREM core fragment or TREM fragment comprises comprising an isoacceptor tRNA moiety having an anticodon that pairs with the SMC, to the subject,

thereby treating the subject.

165. A method of selecting a therapy for a subject having an endogenous open reading frame (ORF) which comprises a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

wherein the presence of the codon having the first sequence is indicative that said subject is likely to be a responder to the therapy, or said subject will respond, or will likely respond, to the therapy,

thereby selecting the therapy.

166. A method of selecting a therapy for a subject having an endogenous open reading frame (ORF) comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject, and

wherein the presence of SMC is indicative that said subject is likely to be a responder to the therapy, or said subject will respond, or will likely respond, to the therapy,

thereby selecting the therapy.

167. A method of evaluating a subject having an endogenous open reading frame (ORF) comprising a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

identifying the subject as having a codon having the first sequence,

thereby evaluating the subject.

168. A method of evaluating a subject having an endogenous open reading frame (ORF) comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject; and

identifying the subject as having a SMC,

thereby evaluating the subject.

169. The method of any one of embodiments 126-168, wherein the TREM, TREM core fragment or TREM fragment does not comprise an anticodon that pairs with a stop codon.
170. The method of any one of embodiments 126-169, wherein: (a) the ORF codon having the first sequence; or (b) the SMC; is other than a stop codon, e.g., TAA, TGA or TAG.
171. The method of any one of embodiments 126-170, wherein the TREM, TREM core fragment or TREM fragment comprises a canonical anti-codon/charging site combination.
172. The method of any one of embodiments 126-171, wherein: (a) the ORF codon having the first sequence; or (b) the SMC; has a mutation, e.g., a SNP, in the first position of said codon.
173. The method of any one of embodiments 126-171, wherein: (a) the ORF codon having the first sequence; or (b) the SMC; has a mutation, e.g., a SNP, in the second position of said codon.
174. The method of any one of embodiments 126-171, wherein: (a) the ORF codon having the first sequence; or (b) the SMC; has a mutation, e.g., a SNP, in the third position of said codon.
175. The method of any one of embodiments 126-174, wherein the first tRNA moiety comprises an endogenous tRNA, and the second tRNA moiety comprises an endogenous tRNA, e.g., wherein the cell or subject has not been contacted with a composition comprising a TREM.
176. The method of any one of embodiments 126-174, wherein one of the first tRNA moiety and the second tRNA moiety comprises an endogenous tRNA and a TREM.
177. The method of any one of embodiments 126-176, wherein: (a) the ORF codon having the first sequence; or (b) the SMC; in the absence of contact with the composition comprising a TREM, is associated with a phenotype, e.g., an unwanted phenotype, e.g., a disorder or symptom, e.g., a disorder or symptom chosen from Table 1.
178. The method of embodiment 177, wherein the disorder or symptom is chosen from a disease group provided in Table 1, e.g., cardiovascular, dermatology, endocrine, immunology, neurology, oncology, ophthalmology, or respiratory.
179. A method of modulating a production parameter of an RNA, or a protein encoded by an RNA, in a target cell or tissue, comprising:

providing, e.g., administering, to the target cell or tissue, or contacting the target cell or tissue with, an effective amount of a TREM composition comprising a TREM of any one of embodiments 1-8, the TREM core fragment of embodiment 22, or the TREM fragment of embodiment 37, which TREM, TREM core fragment or TREM fragment corresponds to a contextually-rare codon (“con-rare codon”) of the RNA,

thereby modulating the production parameter of the RNA, or protein encoded by the RNA in the target cell or tissue.

180. The method of embodiment 179, wherein the target cell or tissue is obtained from a subject.
181. The method of embodiment 179, comprising administering the TREM composition to a subject.
182. The method of embodiment 181, comprising contacting the TREM composition with the target tissue or cell ex vivo.
183. The method of embodiment 182, comprising introducing the ex vivo-contacted target tissue or cell into a subject, e.g., an allogeneic or autologous subject.
184. The method of any one of embodiments 179-183, wherein the target cell or tissue is a specific or selected target cell or tissue, e.g., a cell or tissue type in a particular developmental stage; a cell or tissue type in a particular disease state; or a cell present in a particular extracellular milieu.
185. The method of any one of embodiments 179-184, wherein the production parameter comprises an expression parameter or a signaling parameter, e.g., as described herein.
186. The method of any one of embodiments 179-185, wherein the production parameter of the RNA is modulated, e.g., an RNA that can be translated into a polypeptide, e.g., a messenger RNA.
187. The method of embodiment 186, wherein the production parameter of the RNA is increased or decreased.
188. The method of any one of embodiments 179-187, wherein the production parameter of the protein encoded by the RNA is modulated.
189. The method of embodiment 188, wherein the production parameter of the protein is increased.
190. The method of embodiment 188, wherein the production parameter of the protein is decreased.
191. A method of determining the presence of a nucleic acid sequence, e.g., a DNA or RNA, having a contextually-rare codon (“con-rare codon nucleic acid sequence”), comprising:

acquiring knowledge of the presence of the con-rare codon nucleic acid sequence in a sample from a subject, e.g., a target cell or tissue sample,

wherein responsive to the acquisition of knowledge of the presence of the con-rare codon nucleic acid sequence:

(1) the subject is classified as being a candidate to receive administration of an effective amount of a TREM composition comprising a TREM of any one of embodiments 1-8, the TREM core fragment of embodiment 22, or the TREM fragment of embodiment 37, which TREM, TREM core fragment or TREM fragment corresponds to a contextually-rare codon (“con-rare codon”) of the nucleic acid sequence; or

(2) the subject is identified as likely to respond to a treatment comprising the TREM composition.

192. A method of treating a subject having a disease associated with a contextually-rare codon (“con-rare codon”), comprising:

acquiring knowledge of the presence of a nucleic acid sequence, e.g., a DNA or RNA, having the con-rare codon (“con-rare codon nucleic acid sequence”) in a target cell or tissue sample from the subject; and

administering to the subject an effective amount of a TREM composition comprising a TREM of any one of embodiments 1-8, the TREM core fragment of embodiment 22, or the TREM fragment of embodiment 37, which TREM, TREM core fragment or TREM fragment corresponds to the con-rare codon of the nucleic acid sequence,

thereby treating the disease in the subject.

193. A method of providing a tRNA effector molecule (TREM) to a subject, comprising:

providing, e.g., administering, to the subject, an effective amount of a TREM composition comprising a TREM of any one of embodiments 1-8, the TREM core fragment of embodiment 22, or the TREM fragment of embodiment 37, which TREM, TREM core fragment or TREM fragment corresponds to a contextually-rare codon (“con-rare codon”) for a nucleic acid sequence in a target cell or tissue in the subject,

thereby providing a TREM composition to the subject.

194. A method of manufacturing a tRNA effector molecule (TREM) composition comprising:

identifying a TREM corresponding to a contextually-rare (con-rare) codon;

combining the TREM with a component, e.g., a carrier or excipient.

thereby manufacturing a TREM composition.

195. The method of any one of embodiments 179-194, wherein the method comprises acquiring a value for a con-rare codon in the nucleic acid sequence, e.g., DNA or RNA, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon;

196. The method of embodiment 195, wherein (1) comprises determining the presence or absence of a con-rare codon.
197. The method of embodiment 196, wherein a determination of the availability of a tRNA comprises acquiring a measure of one, two, three or all of the following parameters:

(a) level of a tRNA corresponding to the con-rare codon (“con-rare codon tRNA”) compared to a tRNA corresponding to a different codon;

(b) function, e.g., polypeptide chain elongation function, of a con-rare codon tRNA compared to a tRNA corresponding to a different codon;

(c) modification, e.g., aminoacylation or post-transcriptional modification, of a con-rare codon tRNA compared to a tRNA corresponding to a different codon; and/or

(d) sequence of a con-rare codon tRNA.

198. The method of embodiment 197, wherein a measure of availability (e.g., level) of a con-rare codon tRNA comprises a measure of the con-rare codon tRNA that is charged, e.g., aminoacylated, compared to: (1) the proportion of the con-rare codon tRNA that is not charged; or (2) the proportion of charged tRNA corresponding to a different codon.
199. The method of any one of embodiments 195-198, wherein responsive to said value, the target cell, or tissue, is identified as having a nucleic acid sequence having a con-rare codon (“con-rare codon nucleic acid sequence”) or an RNA having a con-rare codon (“con-rare codon RNA”).
200. The method of any one of embodiments 195-198, wherein responsive to said value, the RNA is identified as, an RNA having a con-rare codon.
201. The method of any one of embodiments 179-200, wherein the target cell or tissue is identified as having an RNA having a con-rare codon.
202. The method of any one of embodiments 179-201, wherein the nucleic acid sequence, e.g., DNA or RNA, is identified as, a nucleic acid sequence having a con-rare codon (“con-rare codon nucleic acid sequence”) or an RNA having a con-rare codon (“con-rare codon RNA”).
203. The method of any one of embodiments 179-202, wherein the nucleic acid sequence (e.g., DNA or RNA) is, or is identified as, a nucleic acid sequence (e.g., DNA or RNA) having a plurality of con-rare codons.
204. The method of any one of embodiments 179-203, wherein the nucleic acid sequence (e.g., DNA or RNA) is, or is identified as, a nucleic acid sequence (e.g., DNA or RNA) having a plurality of occurrences of a con-rare codon.
205. The method of any one of embodiments 179-204, wherein the nucleic acid, e.g., RNA is, or is identified as, a nucleic acid, e.g., RNA, having a first tRNA which corresponds to a first con-rare codon; and an additional tRNA, e.g., a second tRNA, which corresponds to a different, e.g., a second, con-rare codon.
206. The method of any one of embodiments 179-205, wherein the nucleic acid sequence (e.g., DNA or RNA) is, or is identified as, a nucleic acid sequence (e.g., DNA or RNA), having multiple occurrences of the first tRNA which corresponds to a first con-rare codon.
207. The method of embodiment 205 or 206, wherein the nucleic acid sequence (e.g., DNA or RNA) is, or is identified as, a nucleic acid sequence (e.g., DNA or RNA) having multiple occurrences of the additional tRNA, e.g., a second tRNA, which corresponds to a different, e.g., a second, con-rare codon.
208. The method of any one of embodiments 179-207, wherein modulation of a production parameter of the con-rare codon RNA comprises increasing a production parameter, e.g., an expression parameter or signaling parameter of the protein encoded by the con-rare codon RNA, e.g., increasing the expression level of the protein encoded by the con-rare codon RNA.
209. The method of any one of embodiments 179-208, wherein modulation of a production parameter of the con-rare codon RNA comprises decreasing a production parameter, e.g., an expression parameter or signaling parameter, of the protein encoded by the con-rare codon RNA, e.g., decreasing the expression level of the protein encoded by the con-rare codon RNA.
210. The method of any one of embodiments 195-198, wherein a determination of the expression profile (or proteome codon count) of the target cell or tissue, comprises a measure of:

(a) the abundance (e.g., expression) of proteins in a target cell or tissue; and

(b) a protein codon count for expressed proteins in a target cell or tissue.

211. The method of any one of embodiments 179-210, wherein the target cell or tissue is identified as comprising a con-rare-codon nucleic acid, e.g., RNA.
212. The method of any one of embodiments 179-211, wherein, the con-rare codon meets a reference value for one or more of the following:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon;

213. The method of embodiment 212, wherein the con-rare-codon meets a reference value for two of (1)-(5).
214. The method of embodiment 212, wherein the con-rare-codon meets a reference value for three of (1)-(5).
215. The method of embodiment 212, wherein the con-rare-codon meets a reference value for four of (1)-(5).
216. The method of embodiment 212, wherein the con-rare-codon meets a reference value for all of (1)-(5).
217. The method of embodiment 212, wherein the con-rare-codon meets a reference value for (1).
218. The method of embodiment 212, wherein the con-rare-codon meets a reference value for (2).
219. The method of embodiment 212, wherein the con-rare-codon meets a reference value for (3).
220. The method of embodiment 212, wherein the con-rare-codon meets a reference value for (4).
221. The method of embodiment 212, wherein the con-rare-codon meets a reference value for (5).
222. The method of embodiment 212, wherein the reference value is a pre-determined or pre-selected reference value.
223. The method of embodiment 212, wherein the reference value is determined according to a method described herein.
224. The method of any of embodiments 179-223, wherein at least 10%, 20%, 30%, 40%, 50%, 60%, 70%, 80%, 85%, 90%, 95%, 99%, or 100% (by weight or number) of the TREMs, TREM core fragments or TREM fragments in the TREM composition correspond to a con-rare codon.
225. The method of any one of embodiments 179-224, wherein the TREM composition comprises TREMs, TREM core fragments or TREM fragments that correspond to a plurality of con-rare codons.
226. The method of any of embodiments 179-225, wherein the TREM composition comprises: a first TREM which corresponds to a first con-rare codon; and an additional TREM which corresponds to a different con-rare codon.
227. The method of any of embodiments 179-226, wherein the TREM composition comprises: a first TREM which corresponds to a first con-rare codon; and a second TREM which corresponds to a second con-rare codon.
228. The method of any of embodiments 179-227, wherein the TREM composition comprises: a first TREM which corresponds to a first con-rare codon; a second TREM which corresponds to a second con-rare codon; and a third TREM which corresponds to a third con-rare codon.
229. The method of any of embodiments 179-228, wherein the TREM composition comprises: a first TREM which corresponds to a first con-rare codon; a second TREM which corresponds to a second con-rare codon; a third TREM which corresponds to a third con-rare codon; and a fourth TREM which corresponds to a fourth con-rare codon.
230. The method of any of embodiments 179-229, wherein the TREM composition comprises: a first TREM which corresponds to a first con-rare codon; a second TREM which corresponds to a second con-rare codon; a third TREM which corresponds to a third con-rare codon; a fourth TREM which corresponds to a fourth con-rare codon; and a fifth TREM which corresponds to a fifth con-rare codon.
231. The method of any one of embodiments 226-230, wherein at least 10%, 20%, 30%, 40%, 50%, 60%, 70%, 80%, 85%, 90%, 95%, 99%, or 100% (by weight or number) of the TREMs in the TREM composition correspond to the first con-rare codon.
232. The method of any one of embodiments 226-231, wherein at least 10%, 20%, 30%, 40%, 50%, 60%, 70%, 80%, 85%, 90%, 95%, 99%, or 100% (by weight or number) of the TREMs in the TREM composition correspond to the additional, e.g., second, third, fourth or fifth, con-rare codon.
233. The method of any of embodiments 179-232, wherein at least 10%, 20%, 30%, 40%, 50%, 60%, 70%, 80%, 85%, 90%, 95%, 99%, or 100% (by weight or number) of the TREMs in the composition are charged.
234. The method of any of embodiments 179-233, wherein the TREM composition comprises a first TREM which corresponds to a first con-rare codon and an additional TREM, e.g., a second, third, fourth, or fifth TREM, which corresponds to a different, e.g., second, third, fourth, or fifth, con-rare codon, and wherein at least 10%, 20%, 30%, 40%, 50%, 60%, 70%, 80%, 85%, 90%, 95%, 99%, or 100% (by weight or number) of the first TREM in the composition is charged.
235. The method of embodiment 234, wherein at least 10%, 20%, 30%, 40%, 50%, 60%, 70%, 80%, 85%, 90%, 95%, 99%, or 100% (by weight or number) of the additional TREM e.g., second, third, fourth, or fifth TREM, in the composition is charged.
236. The method of any of embodiments 179-235, wherein at least 10%, 20%, 30%, 40%, 50%, 60%, 70%, 80%, 85%, 90%, 95%, 99%, or 100% (by weight or number) of the TREMs in the preparation are of the same iso-decoder isotype.
237. The method of any one of embodiments 179-236, wherein the TREM composition comprises: a first TREM which corresponds to a first con-rare codon; and an additional TREM which corresponds to the first con-rare codon, e.g., the first TREM and the additional TREM are of the same iso-decoder isotype.
238. The method of any one of embodiments 179-237, wherein the TREM composition comprises: a first TREM which corresponds to a first con-rare codon; and a second TREM which corresponds to the first con-rare codon, e.g., the first TREM and the second TREM are of the same iso-decoder isotype.
239. The method of any one of embodiments 179-238, wherein at least 10%, 20%, 30%, 40%, 50%, 60%, 70%, 80%, 85%, 90%, 95%, 99%, or 100% (by weight or number) of the TREMs in the TREM composition correspond to the first con-rare codon.
240. The method of any one of embodiments 179-239, wherein at least 10%, 20%, 30%, 40%, 50%, 60%, 70%, 80%, 85%, 90%, 95%, 99%, or 100% (by weight or number) of the TREMs in the TREM composition correspond to the additional, e.g., second or third, con-rare codon, e.g., the first TREM and the additional TREM are of the same iso-decoder isotype.
241. The method of any one of embodiments 179-240, wherein at least 10%, 20%, 30%, 40%, 50%, 60%, 70%, 80%, 85%, 90%, 95%, 99%, or 100% (by weight or number) of the TREMs in the composition are charged.
242. The method of any one of embodiments 179-241, wherein the TREM composition comprises a first TREM which corresponds to a first con-rare codon, and an additional TREM, e.g., a second or third TREM, which corresponds to the first con-rare codon, and wherein at least 10%, 20%, 30%, 40%, 50%, 60%, 70%, 80%, 85%, 90%, 95%, 99%, or 100% (by weight or number) of the first TREM in the composition is charged.
243. The method of embodiment 242, wherein at least 10%, 20%, 30%, 40%, 50%, 60%, 70%, 80%, 85%, 90%, 95%, 99%, or 100% (by weight or number) of the additional TREM e.g., second or third TREM, in the composition is charged.
244. The method of any one of embodiments 179-243, wherein the cell is a host cell.
245. The method of any one of embodiments 179-244, wherein the cell is a mammalian cell, e.g., a human cell, a murine cell, or a rodent cell.
246. The method of any one of embodiments 179-245, wherein the cell is a non-mammalian cell, e.g., a bacterial cell, an insect cell or a yeast cell.
247. The method of any one of embodiments 179-246, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
248. The method of any one of embodiments 179-247, wherein the cell comprises an exogenous nucleic acid sequence.
249. The method of any one of embodiments 179-248, wherein the cell is autologous to the exogenous nucleic acid sequence.
250. The method of any one of embodiments 179-249, wherein the cell is allogeneic to the exogenous nucleic acid sequence.
251. The method of any one of embodiments 248-250, wherein the exogenous nucleic acid sequence (e.g., DNA or RNA) comprises a con-rare codon.
252. The method of any one of embodiments 248-251, wherein administration of a TREM composition corresponding to the con-rare codon to the cell, modulates a production parameter, e.g., expression parameter or signaling parameter, of a product, e.g., RNA or polypeptide, of the exogenous nucleic sequence.
253. The method of any one of embodiments 248-251, wherein administration of a TREM composition corresponding to the con-rare codon to the cell, increases a production parameter, e.g., expression parameter or signaling parameter, of a product, e.g., RNA or polypeptide, of the exogenous nucleic sequence.
254. The method of any one of embodiments 248-251, wherein administration of a TREM composition corresponding to the con-rare codon, to the cell decreases a production parameter, e.g., expression parameter or signaling parameter, of a product, e.g., RNA or polypeptide, of the exogenous nucleic sequence.
255. The method of any one of embodiments 179-254, wherein the modulation, increase or decrease in production parameter, is compared to an otherwise similar cell, which: (1) is not contacted with the TREM composition; (2) does not comprise an exogenous nucleic acid sequence; or (3) comprises an exogenous nucleic acid sequence which does not comprise a con-rare codon.
256. A method of modulating a production parameter of an RNA, or a protein encoded by the RNA, in a cell, comprising:
optionally, acquiring knowledge of the presence of an RNA having a contextually-rare codon (“con-rare codon RNA”) in the cell,

providing to the cell an effective amount of a tRNA corresponding to the con-rare codon RNA,

thereby modulating the production parameter of the RNA, or the protein encoded by the RNA in the cell.

257. A method of modulating a production parameter of an RNA, or a protein encoded by an RNA, in a cell, comprising:

optionally, acquiring knowledge of the presence of an RNA having a contextually-rare codon (“con-rare codon RNA”) in the cell,

modulating a culture parameter such that a production parameter of the RNA or protein encoded by the RNA is modulated.

258. The method of embodiment 256 or 257, wherein acquiring knowledge of the con-rare codon RNA comprises acquiring a value for a con-rare codon in the RNA, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged;

(5) the iso-decoder isotype of the tRNA corresponding to the con-rare codon;

259. The method of embodiment 257 or 258, wherein modulating a culture parameter comprises any one or all of the following:
(i) changing the amount of time a cell is cultured, e.g., increasing or decreasing the time;
(ii) changing the density of cells in the culture, e.g., increasing or decreasing the cell density;
(iii) changing a component of the culture, e.g., adding or removing or changing the concentration of a media component, a nutrient, a supplement, a pH modulator;
(iv) culturing the cell with one or more additional components, e.g., a cell or a purified cell component (e.g., a tRNA), a cell lysate;
(v) changing the temperature at which the cell is cultured, e.g., increasing or decreasing the temperature; or
(vi) changing the size of the vessel in which the cell is cultured in, e.g., increasing or decreasing the size of the vessel.
   260. The method of any of embodiments 256-259, wherein the cell is a host cell.
   261. The method of any one of embodiments 256-260, wherein the cell is a mammalian cell, e.g., a human cell, a murine cell, or a rodent cell.
   262. The method of any one of embodiments 256-260, wherein the cell is a non-mammalian cell, e.g., a bacterial cell, an insect cell or a yeast cell.
   263. The method of any one of embodiments 256-261, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
   264. The method of any one of embodiments 256-263, wherein the cell comprises an exogenous nucleic acid sequence.
   265. The method of any one of embodiments 256-264, wherein the cell is autologous to the exogenous nucleic acid sequence.
   266. The method of any one of embodiments 256-265, wherein the cell is allogeneic to the exogenous nucleic acid sequence.
   267. The method of any one of embodiments 256-266, wherein the exogenous nucleic acid sequence comprises a con-rare codon.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

Table of Contents for Examples

| | |
|---|---|
| Example 1 | Method of synthesizing guanosine |
| | 2′-O-MOE phosphoramidite |
| Example 2 | Method of synthesizing 5,6 dihydrouridine |
| Example 3 | Method of synthesizing a TREM via 5′-Silyl-2′- |
| | Orthoester (2′-ACE) Chemistry |
| Example 4 | Method of synthesizing an arginine TREM |
| | having a 2′-O-MOE modification |
| Example 5 | Method of synthesizing an arginine TREM |
| | having a pseudouridine and a 2′-O-MOE modification |
| Example 6 | Method of synthesizing a glutamine TREM |
| | having a 5,6 dihydrouridine modification |
| Example 7 | Method of synthesizing a glutamine TREM |
| | having a pseudouridine modification |
| Example 8 | Quality control of synthesized TREM |
| | via Mass Spectrometry Analysis |
| Example 9 | Quality control of synthesized TREM |
| | via anion-exchange HPLC |
| Example 10 | Quality control of synthesized TREM |
| | via PAGE Purification and Analysis |
| Example 11 | Deprotection of synthesized TREM |

Con-rare codon identification

| | |
|---|---|
| Example 12 | Quantitative tRNA profiling |
| Example 13 | Quantification of protein expression levels |
| | across cell lines or tissue types |
| Example 14 | Evaluation of contextual rarity and identification |
| | of contextually rare codons |
| Example 15 | Identification of a nucleic acid sequence |
| | having con-rare codons (A) |
| Example 16 | Identification of a nucleic acid sequence |
| | having con-rare codons (B) |
| Example 17 | Exemplary nucleic acid sequence |
| | having con-rare codons |
| Example 18 | Exemplary computational pipeline for |
| | codon modifying a nucleic acid sequence |

Determining the effect of TREM
administration on protein expression

| | |
|---|---|
| Example 19 | Determining that administration of a TREM |
| | affects expression of a protein encoded |
| | by a nucleic acid sequence having a con-rare codon |

Manufacturing and preparation of TREMs

| | |
|---|---|
| Example20 | Manufacture of TREM in a mammalian production |
| | host cell, and use thereof to modulate a cellular function |
| Example 21 | Manufacture of TREM in a mammalian production |
| | host cell, and use thereof to modulate a cellular function |
| Example 22 | Manufacture of TREMs in modified mammalian |
| | production host cell expressing an oncogene |
| Example 23 | Preparation of a TREM production host cell |
| | modified to inhibit a repressor of tRNA synthesis |
| Example 24 | Manufacture of TREMs in modified mammalian |
| | production host cell overexpressing an oncogene |
| | and a tRNA modifying enzyme |

Assays to analyze TREM activity

| | |
|---|---|
| Example 25 | TREM translational activity assay |

Production of TREMs

| | |
|---|---|
| Example 26 | Production of a candidate TREM complementary to |
| | the con-rare codon through mammalian cell purification |
| Example 27 | Production of a candidate TREM complementary to |
| | the con-rare codon through bacterial cell purification |
| Example 28 | Production of a candidate TREM complementary to |
| | the con-rare codon through chemical synthesis |
| Example 29 | Production of a candidate TREM complementary to |
| | the con-rare codon through in vitro transcription |

Assays to analyze TREM activity

| | |
|---|---|
| Example 30 | Assay for the activity of an uncharged |
| | TREM to modulate autophagy |
| Example 31 | Assay for activity of a mischarged TREM (mTREM) |

Identifying SMC-containing proteins that
would benefit from TREM administration

| | |
|---|---|
| Example 32 | Identification of disease-associated SMC that |
| | could be ameliorated by TREM modulation |
| Example 33 | PNPL3A SMC |
| Example 34 | TERT SMC |
| Example 35 | ACHE SMC |
| Example 36 | CFTR SMC |
| Example 37 | MAP3K1 SMC |

Production of TREMs

| | |
|---|---|
| Example 38 | Production of a candidate TREM complementary to |
| | the SMC through mammalian cell purification |
| Example 39 | Production of a candidate TREM complementary to |
| | the SMC through bacterial cell purification |
| Example 40 | Production of a candidate TREM complementary to |
| | the SMC through chemical synthesis |
| Example 41 | Production of a candidate TREM complementary to |
| | the SMC through in vitro transcription |

Modulation of tRNA pool

| | |
|---|---|
| Example 42 | Modulation of a tRNA pool through |
| | TREM administration to a cell |
| Example 43 | Modulation of a tRNA pool through |
| | TREM administration to a cell using liposome |
| Example 44 | Modulation of a tRNA pool through delivery |
| | of TREM-encoding plasmid to a cell |
| Example 45 | Modulation of a tRNA pool through delivery |
| | of a TREM-encoding viral vector to a cell |

Method of making a cell line with an SMC of
interest to test effects of TREM administration

| | |
|---|---|
| Example 46 | System to test the effects of TREM administration |
| | on an SMC containing ORF |

Assays to test TREM administration effects
on characteristics of SMC-containing protein

| | |
|---|---|
| Example 47 | Evaluation of protein expression levels of |
| | SMC-containing ORF with administration of a TREM |
| Example 48 | Modulation of translation rate of SMC-containing |
| | ORF with TREM administration |
| Example 49 | Determining that modulation of a TREM |
| | complementary to the SMC changes the function |
| | of the protein derived from the SMC-containing ORF |
| Example 50 | Determining that modulation of a TREM |
| | complementary to an SMC changes the localization |
| | of the protein derived from the SMC-containing ORF |
| Example 51 | Determining that modulation of a TREM |
| | complementary to an SMC changes folding |
| | of the protein translated from the SMC-containing ORF |
| Example 52 | Determining that modulation of a TREM |
| | complementary to an SMC alters the cellular phenotype |
| | resulting from translation of the SMC-containing ORF |
| Example 53 | Modulation of TREM to ameliorate a |
| | disease state resulting from translation |
| | of the SMC-containing ORF |

### Example 1: Method of Synthesizing Guanosine 2′-O-MOE Phosphoramidite

This example describes the synthesis of guanosine 2′-O-MOE phosphoramidite. Guanosine 2′-O-MOE phosphoramidite is prepared and purified according to previously published procedures (Wen K. et al. (2002) The Journal of Organic Chemistry, 67(22), 7887-7889).

Briefly, guanosine and imidazole are dried by co-evaporation with pyridine, dissolved in dry DMF, and treated with bis(diisopropylchlorosilyl) methane added dropwise at 0° C. The temperature is gradually increased to 25° C. and then held for 5 h. The reaction mixture is poured into ice water, and the precipitated white solid filtered to afford compound 1.

To a solution of compound 1, BrCH2CH2OCH3, and TBAI in DMF at −20° C. is added with sodium bis (trimethylsilyl)amide, and the mixture is stirred for 4 hours under argon. After the reaction is quenched with methanol, the THF is evaporated and the residue is precipitated in ice to furnish compound 2.

TBAF is added to a solution of compound 2 at 25° C. and then the mixture is stirred at 35° C. for 5 hours. The solvent is then evaporated under reduced pressure, and the residue is filtered in a short pad of silica gel using 10% methanol in dichloromethane to afford guanosine 2′-O-MOE phosphoramidite.

### Example 2: Method of Synthesizing a 5,6 dihydrouridine

This example describes the synthesis of 5,6 dihydrouridine. 5,6 dihydrouridine phosphoramidite is prepared and purified according to previously published procedures (Hanze A R. et al., (1967) Journal of the American Chemical Society, 89(25), 6720-6725).

Briefly, oxygen is bubbled through a solution uridine in the presence of platinum black. The reaction is followed by spotting the reaction mixture on silica gel thin layer chromatographic plates and developing in methanol-chloroform (1:1). After 1 hour, the mixture is cooled and centrifuged and the clear liquid lyophilized to yield the 5,6 dihydrouridine product.

### Example 3: Method of Synthesizing a TREM Via 5′-Silyl-2′-Orthoester (2′-ACE) Chemistry

This example describes the synthesis of a TREM via 5′-Silyl-2′-Orthoester (2′-ACE) Chemistry summarized from (Hartsel S A et al., (2005) Oligonucleotide Synthesis, 033-050).

### Protected Ribonucleoside Monomers

5′-O-silyl-2′-O-ACE protected phosphoramidites are prepared and purified according to previously published procedures (Hartsel S A et al., (2005) Oligonucleotide Synthesis, 033-050). Briefly, monomer synthesis begins from standard base-protected ribonucleosides [rA(ibu), rC(acetyl), rG(ibu) and U]. Orthogonal, 5′-silyl-2′-ACE protection and amidite preparation is then accomplished in five general steps:
1. Simultaneous transient protection of the 5′- and 3′-hydroxyl groups with 1,1,3,3tetraispropyldisiloxane (TIPS).
2. Regiospecific conversion of the 2′-hydroxyl to the 2′-O-orthoester using tris(acetoxyethyl)orthoformate (ACE orthoformate).
3. Removal of the 5′,3′-TIPS protection.
4. Introduction of the 5′-O-silyl ether protecting group using benzhydryloxybis-(trimethylsilyloxy)-chlorosilane (BzH-Cl).
5. Phosphitylation of the 3′-OH with bis(N,N′-diisopropylamino)methoxyphosphine.

The fully protected, phosphitylated monomer is an oil. For ease of handling and dissolution, the phosphoramidite solution is evaporated to dryness in a tared flask to enable quantitation of yields. The phosphoramidite oil is then dissolved in anhydrous acetonitrile, distributed into synthesis vials in 1.0-mmol aliquots, and evaporated to dryness under vacuum in the presence of potassium hydroxide (KOH) and P2O5.

### Synthesis of Oligoribonucleosides

TABLE 11

| | | Delivery | Reaction |
|---|---|---|---|
| Synthesis Step | Reagent | Time | Time |
| Deblock | 3% DCA in DCM | 35 | |
| Activator | 0.5M S-ethyl-tetrazole | 6 | |
| Coupling | 0.1M amidite8.0 | 30 | |
| | 0.5M S-ethyl-tetrazole | 8 | 30 |

Repeat Coupling

| | | | |
|---|---|---|---|
| Oxidation | t-Butyl hydroperoxide | 20 | 10 |

Repeat Oxidation Delivery

| | | | |
|---|---|---|---|
| Capping | 1-methylimidazole and | 12 | 10 |
| | acetic anhydride | | |
| Desilylation | TEAHF | 35 | |

5′-silyl-2′-ACE oligoribonucleotide synthesis begins with the appropriately modified 3′-terminal nucleoside attached through the 3′-hydroxyl to a polystyrene support. The solid support contained in an appropriate reaction cartridge is then placed on the appropriate column position on the instrument. A synthesis cycle is created using the delivery times and wait steps outlined in Table 11.
1. Initial detritylation: The first step in the synthesis cycle is the removal of the 5′ O-DMT from the nucleoside-bound polystyrene support using 3% DCA in DCM.
2. Coupling: The 5-ethylthio-1H-tetrazole solution is delivered to the solid support, followed by simultaneous delivery of an equal quantity of activator and phosphoramidite solution. Depending on the desired sequence and synthesis scale, excess activator and activator plus amidite are alternately delivered repeatedly to increase coupling efficiency, which is typically in excess of 99% per coupling reaction. The 5-ethylthio-1H-tetrazole activates coupling by protonating the diisopropyl amine attached to the trivalent phosphorous. Nucleophilic attack of the 5-ethylthio-1H-tetrazole leads to the formation of the tetrazolide intermediate that reacts with the free 5′-OH of the support-bound nucleoside forming the internucleotide phosphite linkage.
3. Oxidation: In the next step of chain elongation, the phosphorous(III) linkage is oxidized for 10-20 s to the more stable and ultimately desired P(V) linkage using t-butylhydroperoxide.
4. Capping: Although delivery of excess activator and phosphoramidite increases coupling efficiency, a small percentage of unreacted nucleoside may remain support-bound. To prevent the introduction of mixed sequences, the unreacted 5′-OH are “capped” or blocked by acetylating the primary hydroxyl. This acetylation is achieved through simultaneous delivery of 1-methylimidazole and acetic anhydride.
5. 5′-Desilylation: Before the next nucleoside in the sequence can be added to the growing oligonucleotide chain, the 5′-silyl group is removed with fluoride ion. This requires the delivery of triethylamine trihydrogenfluoride for 45 s. The desilylation is rapid and quantitative and no wait step is required.
   Steps 2-5 are repeated for each subsequent nucleotide until the desired sequence is constructed.

### Oligonucleotide Deprotection

A two-stage rapid deprotection strategy is employed to remove phosphate backbone protection, release the oligonucleotide from the solid support, and remove the exocyclic amine protecting groups on A, G, and C. The treatment also removes the acetyl moiety from the acetoxyethyl orthoester, resulting in the 2′-bis-hydroxyethyl protected intermediate that is now 10 times more labile to final acid deprotection. In the first deprotection step, S2Na2 is used to selectively remove the methyl protection from the internucleotide phosphate, leaving the oligoribonucleotide attached to the polystyrene support. This configuration allows any residual reagent to be thoroughly washed away before proceeding. Alternatively, a multicolumn, manifold approach can also be used.
1. A syringe barrel is attached to one of the two luer fittings on the synthesis column. 2 mL of the S2Na2 reagent is drawn into a second syringe and attached to the opposite side of the synthesis column. The S2Na2 reagent is gently pushed through the column and into the empty syringe barrel continuing back and forth several times. The column, filled with reagent is allowed to sit at room temperature for 10 min.
2. S2Na2 reagent is removed from the column. Using a clean syringe, the column is washed thoroughly with water. In the second deprotection step, 40% 1-methylamine in water is used to free the oligoribonucleotide from the solid support, deprotect the exocyclic base amines, and deacylate the 2′-orthoester leaving the deprotected species.

### N-Methylamine Deprotection

1. The solid support resin is transferred from the column into a 4-mL vial
2. 2 mL 40% methylamine is added and heated for 12 min at 60° C.
3. The methylamine is removed and is transferred into a fresh vial.
4. The oligonucleotide solution is evaporated to dryness in a SpeedVac or similar device.
   Oligonucleotide yields are measured using an ultraviolet (UV) spectrophotometer (absorbance at 260 nm).

### Example 4: Method of Synthesizing an Arginine TREM Having a 2′-O-MOE Modification

This example describes the synthesis of an Arg TREM having one 2′-O-MOE modification. The 2′-O-MOE modification can be placed on a nucleotide on any domain or linker of the Arg TREM, or at any position in said domain or linker.

A 2′-ACE RNA oligoribonucleotide synthesis is performed on a modified Applied Biosystems 394 DNA/RNA synthesizer or similar instrument. 2′-O-MOE amidites are synthesized as in example 1. An oligonucleotide sequence: GGCUCCGUGGCGCAAUGGAUAGCGCAUUGGACUUCUAAUUCAAAGGUUCCGGGUU CG(A-MOE)GUCCCGGCGGAGUCG is synthesized following the protocol described in example 3. A similar method can be used to add a 2′-O-MOE modification on a TREM specifying any one of the other 19 amino acids.

### Example 5: Method of Synthesizing an Arginine TREM Having a Pseudouridine and a 2′-O-MOE Modification

This example describes the synthesis of an Arg TREM having a pseudouridine and 2′-O-MOE modification. The modification can be placed on a nucleotide on any domain or linker of the Arg TREM, or at any position in said domain or linker.

A 2′-ACE RNA oligoribonucleotide synthesis is performed on a modified Applied Biosystems 394 DNA/RNA synthesizer or similar instrument. 2′-O-MOE amidites are synthesized as in example 1. Pseudouridine (P) amidites are obtained from Glen Research or similar provider. An oligonucleotide sequence: GGCUCCGUGGCGCAAUGGAUAGCGCAPUGGACUUCUAAUUCAAAGGUUCCGGGUU CG(A-MOE)GUCCCGGCGGAGUCG is synthesized following the protocol described in example 3. A similar method can be used to add a pseudouridine and 2′-O-MOE modification on a TREM specifying any one of the other 19 amino acids.

### Example 6: Method of Synthesizing a Glutamine TREM Having a Dihydrouridine Modification

This example describes the synthesis of a Gln TREM having a dihydrouridine modification. The modification can be placed on a nucleotide on any domain or linker of the Gln TREM, or at any position in said domain or linker.

A 2′-ACE RNA oligoribonucleotide synthesis is performed on a modified Applied Biosystems 394 DNA/RNA synthesizer or similar instrument. Dihydrouridine (D) is synthesized as in example 2. An oligonucleotide sequence: GGUUCCAUGGUGUAAUGGDAAGCACUCUGGACUCTGAAUCCAGCGAUCCGAGUUC GAGUCUCGGUGGAACCUCCA is synthesized following the protocol described in example 3. A similar method can be used to add a dihydrouridine modification on a TREM specifying any one of the other 19 amino acids.

### Example 7: Method of Synthesizing a Glutamine TREM Having a Pseudouridine Modification

This example describes the synthesis of a Gln TREM having a pseudouridine modification. The modification can be placed on a nucleotide on any domain or linker of the Gln TREM, or at any position in said domain or linker.

A 2′-ACE RNA oligoribonucleotide synthesis is performed on a modified Applied Biosystems 394 DNA/RNA synthesizer or similar instrument. Pseudouridine (P) amidites are obtained from Glen Research or similar provider. An oligonucleotide sequence: GGUUCCAUGGUGPAAUGGUAAGCACUCUGGACUCTGAAUCCAGCGAUCCGAGUUC GAGUCUCGGUGGAACCUCCA is synthesized following the protocol described in example 3.

A similar method can be used to add a pseudouridine modification on a TREM specifying any one of the other 19 amino acids.

### Example 8: Quality Control of Synthesized TREM Via Mass Spectrometry Analysis

This example describes the quality control of a synthesized TREM via Mass Spectrometry Analysis.

Using the Perceptive Biosystems Voyager-DE BioSpectrometry Workstation, the referenced protocol for mass spectrometry analysis (4-Van Ausdall) is followed. Briefly, a 3-hydroxy picolinic acid matrix is used for sample crystallization. It is prepared by mixing (10:1:1) 3-HPA:picolinic acid:ammonium hydrogen citrate where each component is dissolved in 30% aqueous acetonitrile at a concentration of 50 mg/mL. One optical density unit (ODU) of oligonucleotide is dissolved in the matrix and heated at 55° C. for 10 min. The sample is spotted on a MALDI plate, allowed to dry, and analyzed accordingly. This method allows confirmation of oligonucleotide identity and detection of low-level impurities present in synthetic oligonucleotide samples.

### Example 9: Quality Control of Synthesized TREM Via Anion-Exchange HPLC

This example describes the quality control of a synthesized TREM via anion-exchange HPLC.

Using the Dionex DNA-Pac-PA-100 column, a gradient is employed using HPLC buffer A and HPLC buffer B. 0.5 ODUs of a sample that has been dissolved in H2O or Tris buffer, pH 7.5 is injected onto the gradient. The gradient employed is based on oligonucleotide length and can be applied according to Table 12. The parameters provided in Table 13 can be used to program a linear gradient on the HPLC analyzer.

TABLE 12
Oligonucleotide length
and gradient percentages

| Length | Gradient |
|---|---|
| (bases) | (% B) |
| 0-5 | 0-30 |
| 6-10 | 10-40 |
| 11-16 | 20-50 |
| 17-32 | 30-60 |
| 33-50 | 40-70 |
| >50 | 50-80 |

TABLE 13
Parameters for a linear gradient on HPLC analyzer

| Time | Flow | % Buffer | % Buffer |
|---|---|---|---|
| (min) | (mL/min) | A | B |
| 0 | 1.5 | 100 | 0 |
| 1 | 1.5 | 100 | 0 |
| 3 | 1.5 | 70a | 30a |
| 15 | 1.5 | 40a | 60a |
| 15.5 | 2.5 | 0 | 100 |
| 17 | 2.5 | 0 | 100 |
| 17.25 | 2.5 | 100 | 0 |
| 23 | 2.5 | 100 | 0 |
| s23.1 | 1.5 | 100 | 0 |
| 24 | 1.5 | 100 | 0 |
| 25 | 0.1 | 100 | 0 |

### Example 10: Quality Control of Synthesized TREM Via PAGE Purification and Analysis

This example describes the quality control of a synthesized TREM via PAGE Purification and Analysis. Gel purification and analysis of 2′-ACE protected RNA follows standard protocols for denaturing PAGE (Ellington and Pollard (1998) In Current Protocols in Molecular Biology, Chanda, V). Briefly, the 2′-ACE protected oligo is resuspended in 200 mL of gel loading buffer. Invitrogen™ NuPAGE™ 4-12% Bis-Tris Gels or similar gel is prepared in gel apparatus. Samples are loaded and gel ran at 50-120 W, maintaining the apparatus at 40° C. When complete, the gel is exposed to ultraviolet (UV) light at 254 nm to visualize the purity of the RNA using UV shadowing. If necessary, the desired gel band is excised with a clean razor blade. The gel slice is crushed and 0.3M NaOAc elution buffer is added to the gel particles, and soaked overnight. The mixture is decanted and filtered through a Sephadex column such as Nap-10 or Nap-25.

### Example 11: Deprotection of Synthesized TREM

This example describes the deprotection of a TREM made according to an in vitro synthesis method, e.g., as described in Example 3. The 2′-protecting groups are removed using 100 mM acetic acid, pH 3.8. The formic acid and ethylene glycol byproducts are removed by incubating at 60° C. for 30 min followed by lyophilization or SpeedVac-ing to dryness. After this final deprotection step, the oligonucleotides are ready for use.

### Example 12: Quantitative tRNA Profiling

This Example describes the quantification of tRNA levels in a cell line or tissue type. Transfer RNA levels are determined using Oxford Nanopore direct RNA sequencing, as previously described in Sadaoka et al., Nature Communications (2019) 10, 754.

Briefly, cells transfected with a tRNA molecule are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 30 minutes. The solution is neutralized by the addition of an equal volume of 100 mM Na-acetate/acetic acid (pH 4.8) and 100 mM NaCl, followed by ethanol precipitation. Deacylated sRNA is dissolved in water, and its integrity verified by agarose gel electrophoresis. Deacylated sRNA is then polyadenylated using yeast poly(A) tailing kit per manufacturer's instructions to generate a sRNA polyadenylated pool. Following polyadenylation, a reverse transcription reaction is performed to generate cDNA using SuperScript III Reverse Transcriptase (Thermo Fisher Scientific) or a thermostable group II intron RT (TGIRT, InGex LLC) that is less sensitive to RNA structure and modifications. A sequencing adapter is ligated onto the cDNA mixture by incubating the cDNA mixture with RNA adapter, T4 ligase and ligation buffer following the standard protocol for Oxford Nanopore resulting in a cDNA library. Nanopore sequencing is then performed on the libraries and the sequences are mapped to a genomic database, in this example to the genomic tRNA database, GtRNAdb. The methods described in this example can be adopted for use to evaluate the tRNA pool across cell lines or tissue types.

### Example 13: Quantification of Protein Expression Levels Across Cell Lines or Tissue Types

This Example describes the quantification of protein expression levels across cell lines or tissue types.

### Cell Culture/Sample Preparation

The protein expression levels are monitored using SILAC based mass-spectrometry proteomics, as previously described in Geiger et al., Molecular and Cellular Proteomics (2012) 10, 754.

Briefly, populations of cells are cultured either in media containing isotope-labeled amino acids, such as Lys8 (e.g., 13C615N2-lysine) and Arg10 (e.g., 13C615N4-arginine); or in media containing natural amino acids. The media is further supplemented with 10% dialyzed serum. Cell cultured in media containing isotope-labeled amino acids incorporate the isotope-labeled amino acids into all of the proteins translated after incubation with said isotope-labeled amino acids. For example, all peptides containing a single arginine will be 6 Da heavier in cells cultured in the presence of instead of isotope-labeled amino acid compared to cells cultured with natural amino acids. Cultured are lysed and sonicated. Cell lysates (e.g., about 100 g) are diluted in 8 M urea in 0.1 M Tris-HCl followed by protein digestion with trypsin according to the FASP protocol (Wisniewski, J. R., et al. (2009) Universal sample preparation method for proteome analysis. Nat. Methods 6, 359-362). After an overnight digestion, peptides are eluted from the filters with 25 mM ammonium bicarbonate buffer. From each sample, about 40 ug of peptides are separated into six fractions by strong anion exchange as described previously (Wisniewski, J. R., et al. (2009) Combination of FASP and StageTip-based fractionation allows in-depth analysis of the hippocampal membrane proteome. J. Proteome Res. 8, 5674-5678).

Eluted peptides are concentrated and purified on C18 StageTips, e.g., as described in Rappsilber et al., Nature Protocols (2007).

### LC-MS/MS Analysis

Peptides are separated by reverse-phase chromatography using a nano-flow HPLC (Easy nanoLC, Thermo Fisher Scientific). The high performance liquid chromatography (HPLC) is coupled to an LTQ-Orbitrap Velos mass spectrometer (Thermo Fisher Scientific). Peptides are loaded onto the column with buffer A (0.5% acetic acid) and eluted with a 200 min linear gradient from 2 to 30% buffer B (80% acetonitrile, 0.5% acetic acid). After the gradient the column is washed with 90% buffer B and re-equilibrated with buffer A.

Mass spectra are acquired in a data-dependent manner, with an automatic switch between MS and MS/MS scans using a top 10 method. MS spectra are acquired in the Orbitrap analyzer, with a mass range of 300-1650 Th and a target value of 106 ions. Peptide fragmentation is performed with the HCD method and MS/MS spectra is acquired in the Orbitrap analyzer and with a target value of 40,000 ions. Ion selection threshold is set to 5000 counts. Two of the data sets are acquired with a high field Orbitrap cell in which the resolution is 60,000 instead of 30,000 (at 400 m/z) for the MS scans. In the first of the two replicates with the high field Orbitrap MS/MS scans are acquired with 15,000 resolution, and in the second with 7500 resolution, which is the same as in the standard Orbitrap, but with shorter transients.

### Data Analysis

Raw MS files are analyzed by MaxQuant using standard metrics, e.g., as described in Table 2 of Tyanova S et al. (2016) Nat. Protocols 11(12) pp. 2301-19. Categorical annotation is supplied in the form of Gene Ontology (GO) biological process, molecular function, and cellular component, the TRANSFAC database as well as participation in a KEGG pathway and membership in a protein complex as defined by CORUM.

The methods described in this example can be adopted for use to evaluate the protein expression levels across cell lines or tissue types.

### Example 14: Evaluation of Contextual Rarity and Identification of Contextually Rare Codons

This example describes the method used to determine components of contextual rarity (con-rarity). This method utilizes the cell line or tissue protein expression level determined by proteomics described in Example 13 or taken from literature. This method also utilizes the tRNA profile determined by Nanopore or other tRNA sequencing platform described in the Example 1 or taken from literature.

### Codon Count Per Nucleic Acid Sequence

Using the coding DNA sequence (CDS) defined using National Center for Biotechnology Information (NCBI https://www.ncbi.nlm.nih.gov/) or other database, the protein-coding sequence is segmented into codons and summed per codon to give a codon count per nucleic acid sequence for each codon encoded in the protein-coding sequence.

### Normalized Proteome Codon Count

The codon count per nucleic acid sequence is then multiplied by the corresponding cell line or tissue protein expression level determined by proteomics to give a cell type normalized proteome codon count across the cell line or tissue.

### Con-Rarity

Con-rarity is a function of normalized proteome codon count and the tRNA expression level. In an embodiment, the con-rarity is determined by dividing the normalized proteome codon count by the tRNA expression level determined by Nanopore or other tRNA sequencing experiment. This provides a measure of codon usage that is contextually dependent on the tRNA profile, e.g., tRNA abundance levels. A codon is determined to be contextually rare (con-rare) if the con-rarity meets a reference value, e.g., a pre-determined or pre-selected reference value, e.g., a threshold. In an embodiment, a codon is con-rare if the value of a normalized proteome codon count divided by the tRNA expression level for a particular tRNA meets a pre-determined reference. In an embodiment, the reference value is a value under e.g., 1.5× sigma of the normally fit distribution to that codon frequency.

### Example 15: Identification of a Nucleic Acid Sequence Having Con-Rare Codons (A)

This Example describes the identification of a nucleic acid sequence having con-rare codons. Con-rare codons are identified as described in Example 14.

### Codon Count Per Nucleic Acid Sequence

Using the coding DNA sequences (CDS) defined using National Center for Biotechnology Information (NCBI https://www.ncbi.nlm.nih.gov/) or other database, all human gene sequences are segmented into codons and summed per codon to give a codon count per nucleic acid sequence, e.g., gene.

### Con-Rare Count Per Nucleic Acid Sequence

Each codon, per nucleic acid sequence, is classified as a con-rare codon or a con-abundant codon. The counts for all con-rare codons, for each nucleic acid sequence, are summed and normalized to the sequence length.

### Determining a Nucleic Acid Sequence Having Con-Rare Codons

The con-rare codon count is fit to a normalized distribution. A nucleic acid sequence that meets a reference value, e.g., a pre-determined reference value, is classified as a nucleic acid sequence having con-rare codons. In an embodiment, a nucleic acid sequence is classified as having con-rare codons if it falls above a reference value, e.g., in the upper 3sigma of the normalized distribution. In an embodiment, a nucleic acid sequence having con-rare codons can have one, two, or more (e.g., 3, 4, 5, 6, 7, 8, 9, 10, 20, 30, 50, 100, 200, 500) of the same con-rare codon or different con-rare codons.

### Example 16: Identification of a Nucleic Acid Sequence Having Con-Rare Codons (B)

This Example describes the identification of a nucleic acid sequence having con-rare codons. Con-rare codons are identified as described in the Example 14.

### Codon Count Per Nucleic Acid Sequence

Using the coding DNA sequences (CDS) defined using National Center for Biotechnology Information (NCBI https://www.ncbi.nlm.nih.gov/) or other database, all human gene sequences are segmented into codons and summed per codon to give a codon count per nucleic acid sequence, e.g., gene.

### Determining a Nucleic Acid Sequence Having Con-Rare Codons

Each codon, per nucleic acid sequence, is classified as a con-rare codon or a con-abundant codon. For each con-rare codon, the counts per nucleic acid sequence is fit to a normalized distribution. A nucleic acid sequence that meets a reference value, e.g., a pre-determined reference value, is classified as a nucleic acid sequence having con-rare codons. In an embodiment, a nucleic acid sequence is classified as having con-rare codons, e.g., specified con-rare codons, if it falls e.g., in the upper 3sigma of the normalized distribution. In an embodiment, a nucleic acid sequence having con-rare codons can have one, two, or more (e.g., 3, 4, 5, 6, 7, 8, 9, 10, 20, 30, 50, 100, 200, 500) of the same con-rare codon or different con-rare codons.

### Example 17: Exemplary Nucleic Acid Sequence Having Con-Rare Codons

This Example describes an exemplary nucleic acid sequence having con-rare codons.

The GRK2 nucleic acid sequence encodes the GRK2 protein (G-protein coupled receptor kinase 2). The method of Examples 4 or 5 was used to identify the GRK2 nucleic acid sequence as having con-rare codons. The GRK2 nucleic acid sequence has a coding sequence that has con-rare codons AAG and CTG. The AAG codon codes for lysine and the CTG codon codes for leucine. In an embodiment, under certain cellular conditions, the expression of the GRK2 protein can be affected by the frequency of tRNAs corresponding to one or more con-rare codons in the GRK2 nucleic acid sequence, e.g., CUU-tRNA which corresponds to con-rare codon AAG, and/or CAG-tRNA which corresponds to con-rare codon CTG.

### Example 18: Exemplary Computational Pipeline for Codon Modifying a Nucleic Acid Sequence

This Example describes the computational pipeline that can be utilized to codon modify a nucleic acid sequence.

### Mapping Con-Rare Codons

Con-rarity (determined using the method described in Example 14) is read into the algorithm. Con-rare codons are identified as described in Example 14. For example, a codon is determined to be contextually rare (con-rare) if the con-rarity meets a reference value, e.g., a pre-determined or pre-selected reference value, e.g., a threshold. A corresponding contextually abundant (con-abundant) codon is identified as the most contextually frequent codon that encodes the same amino acid as the con-rare codon (e.g., an isoacceptor or an isodecoder). In an embodiment, a con-rare codon can have more than one corresponding con-abundant codon. In an embodiment, the corresponding con-abundant codon can be utilized to replace a con-rare codon.

### Con-Rare Codon Modification

Each sequence to be modified is read in and segmented into codons. Each codon is then evaluated to determine if it is a con-rare codon. If the codon is identified as a con-rare codon, the codon is replaced, e.g., with a corresponding con-abundant codon. A con-abundant codon is a codon other than a con-rare codon. This process can be repeated for two, three, four, or a portion of, or all of the con-rare codons found in the sequence. The resultant con-rare modified sequence (e.g., also referred to as contextually modified nucleic acid sequence) is then outputted.

### Example 19: Determining that Administration of a TREM Affects Expression of a Protein Encoded by a Nucleic Acid Sequence Having a Con-Rare Codon

This Example describes administration of a TREM to modulate expression levels of a protein encoded by a nucleic acid sequence having a con-rare codon in its coding sequence (CDS).

To create a system in which to study the effects of TREM administration on protein expression levels of a protein encoded by a nucleic acid sequence having a con-rare codon in its CDS, the sequence for the GRK2 gene (GRK2-CCDS8156.1 sequence) is inserted into a plasmid. The plasmid is transfected in the normal human hepatocyte cell line THLE-3. A TREM is delivered to the CCDS8156.1 containing cells. As a control, a population of cells prior to the delivery of the TREM is set aside. In this example, the tRNA-Lys^{CUU} containing an CUU anticodon, that base pairs to the AAG codon, i.e. with the sequence GCCCGGCUAGCUCAGUCGGUAGAGCAUGGGACUCUUAAUCCCAGGGUCGUGGGUU CGAGCCCCACGUUGGGCG is used. A time course is performed ranging from 30 minutes to 6 hours with hour-long interval time points. At each time point, a population of cells that have been delivered the TREM, and a population of cells that have not been exposed to the TREM are trypsinized, washed and lysed. Cell lysates are analyzed by Western blotting and blots are probed with antibodies against the GRK2 protein. A total protein loading control, such as GAPDH, actin or tubulin, is also used.

The methods described in this example can be adopted to evaluate the expression levels of the GRK2 protein in cells endogenously expressing CCDS8156.1.

### Example 20: Manufacture of TREM in a Mammalian Production Host Cell, and Use Thereof to Modulate a Cellular Function

This example describes the manufacturing of a TREM produced in mammalian host cells.

### Plasmid Generation

To generate a plasmid comprising a TREM which comprises a tRNA gene, in this example, tRNAiMet, a DNA fragment containing the tRNA gene (chr6.tRNA-iMet(CAT) with genomic location 6p22.2 and sequence AGCAGAGTGGCGCAGCGGAAGCGTGCTGGGCCCATAACCCAGAGGTCGATGGATCG AAACCATCCTCTGCTA) is PCR-amplified from human genomic DNA using the following primer pairs: 5′-TGAGTTGGCAACCTGTGGTA and 5′-TTGGGTGTCCATGAAAATCA. This fragment is cloned into the pLKO.1 puro backbone plasmid with a U6 promoter (or any other RNA polymerase III recruiting promoter) following the manufacturer's instructions.

### Transfection

1 mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR).

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe or a 2′-OMe nucleic acid that is complementary to a unique region of the TREM being purified, in this example, a probe conjugated to biotin at the 3′ end with the sequence UAGCAGAGGAUGGUUUCGAUCCAUCA, is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Use

One microgram of the test TREM preparation and a control agent are contacted by transfection, electroporation or liposomal delivery, with a cultured cell line, such as a HEP-3B or HEK293T, a tissue or a subject, for a time sufficient for the TREM preparation to modulate a translation level or activity of the cell, relative to the control agent.

### Example 21: Manufacture of TREM in a Mammalian Production Host Cell, and Use Thereof to Modulate a Cellular Function

This example describes the manufacturing of a TREM produced in mammalian host cells.

### Plasmid Generation

To generate a plasmid comprising a TREM which comprises a tRNA gene, in this example, tRNA-iMet-CAT, a DNA fragment containing at least one copy of the tRNA gene with the sequence AGCAGAGTGGCGCAGCGGAAGCGTGCTGGGCCCATAACCCAGAGGTCGATGGATCG AAACCATCCTCTGCTA is synthesized and cloned into the pLKO.1 puro backbone plasmid with a U6 promoter (or any other RNA polymerase III recruiting promoter) following the manufacturer's instructions and standard molecular cloning techniques.

### Transfection

1 mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR) or Nanopore sequencing.

### Purification

At the optimized harvest timepoint, the cells are lysed and separation from the lysate of RNAs smaller than 200 nucleotides is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe or a 2′-OMe nucleic acid that is complementary to a unique region of the TREM being purified. In this example, a probe with the sequence 5′biotin-TAGCAGAGGATGGTTTCGATCCATCA is used to purify the TREM comprising tRNA-iMet (CAT). The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner.

The beads are then washed until the absorbance of the wash solution at 260 nm is close to zero. Alternatively, the beads are washed three times and the final wash is examined by UV spectroscopy to measure the amount of nucleic acid present in the final wash. The TREM retained on the beads are eluted three times using RNase-free water which can be pre-heated to 80° C., and then admixed with a pharmaceutically acceptable excipient to make a test TREM product.

### Use

One microgram of the test TREM preparation and a control agent are contacted by transfection, electroporation or liposomal delivery, with a cultured cell line, such as HeLa, HEP-3B or HEK293T, a tissue or a subject, for a time sufficient for the TREM preparation to modulate a translation level or activity of the cell, relative to the control agent.

### Example 22: Manufacture of TREMs in Modified Mammalian Production Host Cell Expressing an Oncogene

This example describes the manufacturing of a TREM in mammalian host cells modified to overexpress myc.

### Plasmid Generation and Host Cell Modification

To make the production host cells for this example, HeLa cells (ATCC® CCL-2™) or HEP-3B cells (ATCC® HB-8064™) are transfected with a plasmid containing the gene sequence coding for the c-myc oncogene protein (e.g., pcDNA3-cmyc (Addgene plasmid #16011)) using routine molecular biology techniques. The resulting cell line is referred to herein as HeLamyc+ host cells or HEP-3Bmyc+ host cells.

### Preparation of TREM Expressing Lentivirus

To prepare a TREM expressing lentivirus, HEK293T cells are co-transfected with 3 μg of each packaging vector (pRSV-Rev, pCMV-VSVG-G and pCgpV) and 9 μg of the plasmid comprising a TREM as described in Example 9, using Lipofectamine 2000 according to manufacturer's instructions. After 24 hours, the media is replaced with fresh antibiotic-free media and after 48 hours, virus-containing supernatant is collected and centrifuged for 10 min at 2000 rpm before being filtered through a 0.45 m filter.

Transduction of host cells with TREM expressing lentivirus 2 mL of virus prepared as described above is used to transduce 100,000 HeLamyc+ host cells or HEP-3Bmyc+ host cells, in the presence of 8 μg/mL polybrene. Forty-eight hours after transduction, puromycin (at 2 μg/mL) antibiotic selection is performed for 2-7 days alongside a population of untransduced control cells.

The TREMs are isolated, purified, and formulated as described in Example 20 or 21 to result in a composition comprising a TREM or preparation comprising a TREM.

### Example 23: Preparation of a TREM Production Host Cell Modified to Inhibit a Repressor of tRNA Synthesis

This example describes the preparation of Hek293Maf-/TRM1 cells for the production of a TREM.

Maf1 is a repressor of tRNA synthesis. A Maf1 knockout HEK293T cell line is generated using standard CRISPR/Cas knockout techniques, e.g., a CRISPR/Cas system can be designed to introduce a frameshift mutation in a coding exon of Maf1 to reduce the expression of Maf1 or knockout Maf1 expression, to generate a Hek293Maf-cell line that has reduced expression level and/or activity of Maf1. This cell line is then transfected with an expression plasmid for modifying enzyme Trm1 (tRNA (guanine26-N2)-dimethyltransferase) such as pCMV6-XL4-Trm1, and selected with a selection marker, e.g., neomycin, to generate a stable cell line overexpressing Trm1 (Hek293Maf-/TRM1 cells).

Hek293Maf-/TRM1 cells can be used as production host cells for the preparation of a TREM as described in any of Examples 20-22.

### Example 24: Manufacture of TREM in Modified Mammalian Production Host Cell Overexpressing an Oncogene and a tRNA Modifying Enzyme

This Example describes the manufacturing of a TREM in mammalian host cells modified to overexpress Myc and Trm1.

### Plasmid Generation

In this example, a plasmid comprising a TREM is generated as described in Example 20 or 21.

### Host Cell Modification, Transduction and Purification

A human cell line, such as HEK293T, stably overexpressing Myc oncogene is generated by transduction of retrovirus expressing the myc oncogene from the pBABEpuro-c-myc^{T58A} plasmid into HEK293T cells. To generate myc-expressing retrovirus, HEK293T cells are transfected using the calcium phosphate method with the human c-myc retroviral vector, pBABEpuro-c-myc^{T58A} and the packaging vector, ψ² vector. After 6 hours, transfection media is removed and replaced with fresh media. After a 24-hour incubation, media is collected and filtered through a 0.45 um filter. For the retroviral infection, HEK293T cells are infected with retrovirus and polybrene (8 ug/ml) using spin infection at 18° C. for 1 hour at 2500 rpm. After 24 hours, the cell culture medium is replaced with fresh medium and 24 hours later, the cells are selected with 2 μg/mL puromycin. Once cells stably overexpressing the oncogene myc are established, they are transfected with a Trm1 plasmid, such as the pCMV6-XL4-Trm1 plasmid, and selected with a selection marker, in this case with neomycin, to generate a stable cell line overexpressing Trm1, in addition to Myc. In parallel, lentivirus to overexpress TREM is generated as described in Example 3 with HEK293T cells and PLKO.1-tRNA vectors. 1×10⁵ cells overexpressing Myc and Trm1 are transduced with the TREM virus in the presence of 8 μg/mL polybrene. Media is replaced 24 hours later. Forty-eight hours after transduction, antibiotic selection is performed with 2 μg/mL puromycin for 2-7 days alongside a population of untransduced control cells. The TREMs are isolated, purified and formulated using the method described in Example 20 or 21 to produce a TREM preparation.

### Example 25: TREM Translational Activity Assays

This example describes assays to evaluate the ability of a TREM to be incorporated into a nascent polypeptide chain.

### Translation of the FLAG-AA-his Peptide Sequence

A test TREM is assayed in an in-vitro translation reaction with an mRNA encoding the peptide FLAG-XXX-His6x, where XXX are 3 consecutive codons corresponding to the test TREM anticodon.

A tRNA-depleted rabbit reticulocyte lysate or human cell lysate (Jackson et al. 2001. RNA 7:765-773) is incubated 1 hour at 30° C. with 10-25 ug/mL of the test TREM in addition to 10-25 ug/mL of the tRNAs required for the FLAG and His tag translation. A different mammalian lysate such as a HEK293T human cell-derived lysate can also be used in this assay. In this example, the TREM used is tRNA-Ile-GAT, therefore the peptide used is FLAG-LLL-His6x and the tRNAs added are tRNA-Ile-GAT, in addition to the following, which are added for translate the peptide FLAG and HIS tags: tRNA-Asp-GAC, tRNA-Tyr-TAC, tRNA-Lys-AAA, tRNA-Lys-AAAG, tRNA-Asp-GAT, tRNA-His-CAT. To determine if the test TREM is functionally able to be incorporated into a nascent peptide, an ELISA capture assay is performed. Briefly, an immobilized anti-His6X antibody is used to capture the FLAG-LLL-His6x peptide from the reaction mixture. The reaction mixture is then washed off and the peptide is detected with an enzyme-conjugated anti-FLAG antibody, which reacts to a substrate in the ELISA detection step. If the TREM produced is functional, the FLAG-LLL-His6 peptide is produced and detection occurs by the ELISA capture assay. The methods described in this example can be adopted for use to evaluate the functionality of the TREM.

### Translational Suppression Assay

This assay describes a test TREM having translational adaptor molecule function by rescuing a suppression mutation and allowing the full protein to be translated. The test TREM, in this example tRNA-Ile-GAT, is produced such that it contains the sequence of the tRNA-Ile-GAT body but with the anticodon sequence corresponding to CUA instead of GAT. HeLa cells are co-transfected with 50 ng of TREM and with 200 ng of a DNA plasmid encoding a mutant GFP containing a UAG stop codon at the S29 position as described in Geslain et al. 2010. J Mol Biol. 396:821-831. HeLa cells transfected with the GFP plasmid alone serve as a negative control. After 24 hours, cells are collected and analyzed for fluorescence recovery by flow cytometry. The fluorescence is read out with an emission peak at 509 nm (excitation at 395 nm). The methods described in this example can be adopted for use to evaluate the functionality of the TREM, or if the TREM can rescue the stop mutation in the GFP molecule and can produce the full-length fluorescent protein.

### In Vitro Translational Assay

This assay describes a test TREM having translational adaptor molecule function by successfully being incorporated into a nascent polypeptide chain in an in vitro translation reaction. First, a rabbit reticulocyte lysate that is depleted of the endogenous tRNA using an antisense or complimentary oligonucleotide which (i) targets the sequence between the anticodon and variable loop; or (ii) binds the region between the anticodon and variable loop is generated (see, e.g., Cui et al. 2018. Nucleic Acids Res. 46(12):6387-6400). 10-25 ug/mL of the test TREM is added in addition to 2 ug/uL of a GFP-encoding mRNA to the depleted lysate. A non-depleted lysate with the GFP mRNA and with or without test TREM added are used as a positive control. A depleted lysate with the GFP mRNA but without the test TREM added is used as a negative control. The progress of GFP mRNA translation is monitored by fluorescence increase on a microplate reader at 37° C. for 3-5 h using λₑₓ485/λₑₘ528. The methods described in this example can be adopted for use to evaluate if the test TREM can complement the depleted lysate and is thus likely functional.

### Example 26: Production of a Candidate TREM Complementary to the Con-Rare Codon Through Mammalian Cell Purification

This example describes the production of a TREM in mammalian host cells.

### Plasmid Generation

To generate a plasmid comprising a TREM which comprises a tRNA gene, in this example, tRNA-Ser-AGA, a DNA fragment containing at least one copy of the tRNA gene with the sequence GTAGTCGTGGCCGAGTGGTTAAGGCGATGGACTAGAAATCCATTGGGGTTTCCCCGC GCAGGTTCGAATCCTGCCGACTACG is synthesized and cloned into the pLKO.1 puro backbone plasmid with a U6 promoter (or any other RNA polymerase III recruiting promoter) following the manufacturer's instructions and standard molecular cloning techniques.

### Transfection

One (1) mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by a quantitative method such as Northern blot, quantitative PCR (q-PCR) or Nanopore sequencing.

### Purification

At the optimized harvest timepoint, the cells are lysed, and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is incubated with annealing buffer and the biotinylated capture probe corresponding to a DNA probe that is complementary to a unique region of the TREM being purified, in this example, a probe with the sequence 3′ biotin-CCAATGGATTTCTATCCATCGCCTTAACCACTCGGCCACGACTACAAAA is used to purify the TREM comprising tRNA-Ser-AGA. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 27: Production of a Candidate TREM Complementary to a Con-Rare Codon Through Bacterial Cell Purification

This example describes the production of a TREM in bacterial host cells.

### Plasmid Generation

To generate a plasmid to produce a TREM in bacteria, a tRNA gene, in this example, a DNA fragment containing at least one copy of the tRNA-Lys-UUU gene with the sequence GCCCGGATAGCTCAGTCGGTAGAGCATCAGACTTTTAATCTGAGGGTCCAGGGTTCA AGTCCCTGTTCGGGCG is synthesized and cloned into a bacterial tRNA expression vector as previously described in Ponchon et al., Nat Protoc 4, 947-959 (2009).

### Transformation

1×10⁹ bacteria grown from TREM expression plasmid transformed competent bacteria will be harvested at different cell density points, in this example OD(600)=0.5, OD(600)=0.7, OD(600)=0.9 to determine the optimal point of TREM expression as determined by a quantitative method such as Northern blot, quantitative PCR (q-PCR) or Nanopore sequencing.

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe that is complementary to a unique region of the TREM being purified, in this example, a probe conjugated to biotin at the 3′ end with the sequence CAGAUUAAAAGUCUG, is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times.

The TREM retained on the beads are eluted three times by adding elution buffer pre-heated to 80° C. and then admixed with a pharmaceutically acceptable excipient to make a test TREM product.

### Example 28: Production of a Candidate TREM Complementary to a Con-Rare Codon Through Chemical Synthesis

This example describes production of a TREM using chemical synthesis.

The TREM, in this example, tRNA-Thr-CGT, is chemically synthesized with the sequence GGCUCUAUGGCUUAGUUGGUUAAAGCGCCUGUCUCGUAAACAGGAGAUCCUGGG UUCGACUCCCAGUGGGGCCUCAA. This TREM is produced by solid-phase chemical synthesis using phosphoroamedite chemistry as previously described, for example as in Zlatev et. al. (2012) Current Protocols, 50 (1), 1.28.1-1.28.16. Briefly, protected RNA phorphoroamedites are sequentially added in a desired order to a growing chain immobilized on a solid support (e.g. controlled pore glass). Each cycle of addition has multiple steps, including: (i) deblocking the DMT group protecting the 5′-hydroxyl of the growing chain, (ii) coupling the growing chain to an incoming phosphoramidite building block, (iii) capping any chain molecules still featuring a 5′-hydroxyl, i.e. those that failed to couple with the desired incoming building block, and (iv) oxidation of the newly formed tricoordinated phosphite triester linkage. After the final building block has been coupled and oxidized, the chain is cleaved from the solid support and all protecting groups except for the DMT group protecting the 5′-hydroxyl are removed. The chain is then purified by RP-HPLC (e.g., DMT-on purification) and the fraction containing the chain is subjected to deprotection of the DMT group under acidic conditions, affording the final TREM. The TREM will feature a 5′-phosphate and a 3′-OH. The TREM is then admixed with a pharmaceutically acceptable excipient to make a test TREM product.

If the TREM needs to be charged, the TREM produced by the chemical synthesis reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with threonyl-tRNA synthetase and threonine, respectively, and then phenol extracted, filtered using a Nuc-trap column, and ethanol precipitated. The TREM is then admixed with a pharmaceutically acceptable excipient to make a test TREM product.

### Example 29: Production of a Candidate TREM Complementary to a Con-Rare Codon Through In Vitro Transcription

This example describes production of a TREM using in vitro transcription (IVT).

The TREM, in this example, tRNA-Leu-CAA, is produced using in vitro transcription with the sequence GUCAGGAUGGCCGAGUGGUCUAAGGCGCCAGACUCAAGUUCUGGUCUCCGUAUG GAGGCGUGGGUUCGAAUCCCACUUCUGACA as previously described in Pestova et al., RNA 7(10):1496-505 (2001). Briefly, a DNA plasmid containing a bacteriophage T7 promoter followed by the tRNA-Leu-CAA gene sequence is linearized and transcribed in vitro with T7 RNA polymerase at 37° C. for 45 min and then phenol extracted, filtered using a Nuc-trap column, and ethanol precipitated. The TREM is then admixed with a pharmaceutically acceptable excipient to make a test TREM product. Optionally, before admixing with a pharmaceutically acceptable excipient, the TREM is heated and cooled to refold the TREM.

If the TREM needs to be charged, the TREM produced by the IVT reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with leucyl-tRNA synthetase and leucine, respectively, and then phenol extracted, filtered using a Nuc-trap column, and ethanol precipitated. The TREM is then admixed with a pharmaceutically acceptable excipient to make a test TREM product.

### Example 30: Assay for the Activity of an Uncharged TREM to Modulate Autophagy

This example describes an assay to test an uncharged TREM for ability to modulate, e.g., induce, autophagy, e.g., the ability to activate GCN2-dependent stress response (starvation) pathway signaling, inhibit mTOR or activate autophagy.

A test uncharged TREM (uTREM) preparation is delivered to HEK293T or HeLa cells through transfection or liposomal delivery. Once the uTREM is delivered, a time course is performed ranging from 30 minutes to 6 hours with hour-long interval time points. Cells are then trypsinized, washed and lysed. The same procedure is executed with a charged control TREM as well as random RNA oligos as controls. Cell lysates are analyzed by Western blotting and blots are probed with antibodies against known readouts of GCN2 pathway activation, mTOR pathway inhibition or autophagy induction, including but not limited to phospho-eIF2a, ATF4, phospho-ULK1, phospho-4EBP1, phospho-eIF2a, phospho-Akt and phospho-p70S6K. A total protein loading control, such as GAPDH, actin or tubulin, as well as the non-modified (i.e. non-phosphorylated) signaling protein, i.e. using eIF2a as a control for phospho-eIF2a, are probed as loading controls. The methods described in this example can be adopted for use to evaluate activation of GCN2 starvation signaling pathway, autophagy pathway and/or inhibition of the mTOR pathway upon TREM delivery.

### Example 31: Assay for Activity of a Mischarged TREM (mTREM)

This example describes an assay to test the functionality of a mTREM produced in a cell system using plasmid transfection followed by in vitro mischarging.

In this example, an mTREM can translate a mutant mRNA into a wild type (WT) protein by incorporation of the WT amino acid in the protein despite an mRNA containing a mutated codon. GFP mRNA molecules with either a T203I or E222G mutation, which prevent GFP excitation at the 470 nm and 390 nm wavelengths, respectively, are used for this example. GFP mutants which prevent GFP fluorescence could also be used as reporter proteins in this assay. Briefly, an in vitro translation assay is used, using a rabbit reticulocyte lysate containing the GFP E222G mutated mRNA (GAG→GGG mutation) and an excess of the mTREM, in this case tRNA-Glu-CCC. As a negative control, no mischarged TREM is added to the reaction. The methods described in this example can be adopted for use to evaluate the functionality of the mTREM.

### Example 32: Identification of Disease-Associated SMC that could be Ameliorated by TREM Modulation

This example describes SMC-containing protein target selection for TREM-based therapy. SMCs can be understood as mutations that are informationally silent, they change the codon sequence to a synonymous codon but may have an effect on a translational or post-translational property. The selection method was segmented into three progressive selection steps (1) SMC identification, (2) examination of tRNA frequency and (3) annotation of disease relevance. These steps are described in further detail below.

### SMC Identification

A curated inclusive list of all known SNPs was utilized as a starting point for SMC selection. In this example, the dbSNP NCBI mutation database (https://www.ncbi.nlm.nih.gov/and FTP site ftp://ftp.ncbi.nih.gov/snp/organisms/) was filtered to select for a SMC, also known as synonymous SNPs (i.e. single nucleotide changes in the coding sequence not causing a change in the amino acid). Briefly, the mutated sequences were aligned to the human genome (here GRCh38p7) and the SNPS were classified into variant and mutation types, such as: non-coding-variant or coding-variant; and synonymous or non-synonymous mutations. Those classified as coding variants with synonymous mutations were designated as SMCs and taken forward into the next selection.

Examination of tRNA Frequency

For each SMC, the corresponding tRNA to each wildtype and mutated codon (SMC) was identified. The abundance of the tRNA for each of the wildtype and mutated codon (SMC) was determined from tRNA-sequencing data. In this example, the tRNA-seq previously determined from HEK293T cells (Zheng et al., Nature Methods 12, 835-837 (2015)) was utilized. SNPs that have differences, e.g., large differences, such as >10× change, in the tRNA abundance are prioritized into the next selection.

### Determining Disease Relevance

SNP IDs were mapped to a collection of known disease associated SNPs to determine which SNPs have disease correlation. In this example we utilize the GWAS (Genome Wide Associate Studies) (https://www.ebi.ac.uk/gwas/) or a similar resource to determine which SNPs have known disease correlations. Those with therapeutically relevant disease correlations (e.g., oncogenic, or relevant to a neurological disorder) were taken forward to the next step.

### Final Selection

The filtered list of SMCs contains SMCs in coding regions that: (1) do not alter the coding sequence of an amino acid; (2) have difference, e.g., large difference, in tRNA population; and (3) have disease relevancy. In this example, the final selection is done based upon a disease of interest, e.g., pancreatic cancer. The BCAR1 gene is, e.g., known to be associated with pancreatic cancer, and has a SNP (rs7190458) that causes a change from codon CUC to CUU. This coding sequence change results in a corresponding change in incorporated TREMs. In some embodiments, the mutated incorporated TREMs has, e.g., about a 100× fold decrease in abundance making it a potential target for upregulation and/or amelioration of the disease phenotype.

### Example 33: PNPL3A SMC

The method of Example 32 was used to identify an SMC in the PNPL3A gene. The PNPL3A gene has a rs738408 polymorphism that was identified as a predisposing factor for nonalcoholic fatty liver disease, fibrosis and elevation of serum alanine transaminase in the human. The rs738408 polymorphism is a SMC as it is located in an ORF and changes the codon from CCC to CCU. Both the CCC and CCU codons code for the proline amino acid, resulting in an identical polypeptide sequence at that position of the chain as that of the wildtype PNPL3A ORF. This polypeptide chain is the adiponutrin protein.

### Example 34: TERT SMC

The method of Example 32 was used to identify an SMC in the TERT gene. The TERT gene has a rs2736098 polymorphism that was identified as a susceptibility factor for pancreatic cancer and non-small cell lung carcinoma in the human. The rs2736098 polymorphism is a SMC as it is located in an ORF and changes the codon from GCG to GCA. Both the GCG and GCA codons code for the alanine amino acid, resulting in an identical polypeptide sequence at that position of the chain as that of the wildtype TERT ORF. This polypeptide chain is the telomerase reverse transcriptase protein.

### Example 35: ACHE SMC

The method of Example 32 was used to identify an SMC in the ACHE gene. The ACHE gene has a rs7636 polymorphism that was identified as a susceptibility factor for Type 2 Diabetes in Asian populations. The rs7636 polymorphism is a SMC as it is located in an ORF and changes the codon from CCC to CCT. Both the CCC and CCT codons code for the proline amino acid, resulting in an identical polypeptide sequence at that position of the chain as that of the wildtype ACHE ORF. This polypeptide chain is the acetylcholinesterase (AChE) protein, which is the primary enzyme responsible for the hydrolytic metabolism of the neurotransmitter acetylcholine (ACh) into choline and acetate.

### Example 36: CFTR SMC

The method of Example 32 was used to identify an SMC in the CFTR gene. The CFTR gene has a rs1042077 polymorphism that is present in patients with CFTR-related disorders. The rs1042077 polymorphism is a SMC as it is located in an ORF and changes the codon from ACT to ACG. Both the ACT and ACG codons code for the threonine amino acid, resulting in an identical polypeptide sequence at that position of the chain as that of the wildtype CFTR ORF. This polypeptide chain is the cystic fibrosis transmembrane conductance regulator (CFTR).

### Example 37: MAP3K1 SMC

The method of Example 32 was used to identify an SMC in the MAP3K1 gene. The MAP3K1 gene has a rs2229882 polymorphism that was identified as a susceptibility factor for the early onset of breast cancer. The rs2229882 polymorphism is a SMC as it is located in an ORF and changes the codon from ACC to ACT. Both the ACC and ACT codons code for the threonine amino acid, resulting in an identical polypeptide sequence at that position of the chain as that of the wildtype MAP3K1 ORF. This polypeptide chain is the Mitogen-Activated Protein Kinase Kinase Kinase 1 (MAP3K1), which is serine/threonine kinase that regulates the ERK and JNK MAPK pathways as well as the transcription factor NF-kappa-B pathway.

### Example 38: Production of a Candidate TREM Complementary to the SMC Through Mammalian Cell Purification

This example describes the production of a TREM in mammalian host cells.

### Plasmid Generation

To generate a plasmid comprising a TREM which comprises a tRNA gene, in this example, tRNA-Ser-AGA, a DNA fragment containing at least one copy of the tRNA gene with the sequence GTAGTCGTGGCCGAGTGGTTAAGGCGATGGACTAGAAATCCATTGGGGTTTCCCCGC GCAGGTTCGAATCCTGCCGACTACG is synthesized and cloned into the pLKO.1 puro backbone plasmid with a U6 promoter (or any other RNA polymerase III recruiting promoter) following the manufacturer's instructions and standard molecular cloning techniques.

### Transfection

One (1) mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by a quantitative method such as Northern blot, quantitative PCR (q-PCR) or Nanopore sequencing.

### Purification

At the optimized harvest timepoint, the cells are lysed, and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is incubated with annealing buffer and the biotinylated capture probe corresponding to a DNA probe that is complementary to a unique region of the target TREM being purified, in this example, a probe with the sequence 3′ biotin-CCAATGGATTTCTATCCATCGCCTTAACCACTCGGCCACGACTACAAAA is used to purify the TREM comprising tRNA-Ser-AGA. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 39: Production of a Candidate TREM Complementary to the SMC Through Bacterial Cell Purification

This example describes the production of a TREM in bacterial host cells.

### Plasmid Generation

To generate a plasmid to produce a TREM in bacteria, a tRNA gene, in this example, a DNA fragment containing at least one copy of the tRNA-Lys-UUU gene with the sequence GCCCGGATAGCTCAGTCGGTAGAGCATCAGACTTTTAATCTGAGGGTCCAGGGTTCA AGTCCCTGTTCGGGCG is synthesized and cloned into a bacterial tRNA expression vector as previously described in Ponchon et al., Nat Protoc 4, 947-959 (2009).

### Transformation

1×10⁹ bacteria grown from TREM expression plasmid transformed competent bacteria will be harvested at different cell density points, in this example OD(600)=0.5, OD(600)=0.7, OD(600)=0.9 to determine the optimal point of TREM expression as determined by a quantitative method such as Northern blot, quantitative PCR (q-PCR) or Nanopore sequencing.

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe that is complementary to a unique region of the target TREM being purified, in this example, a probe conjugated to biotin at the 3′ end with the sequence CAGAUUAAAAGUCUG, is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 40: Production of a Candidate TREM Complementary to the SMC Through Chemical Synthesis

This example describes production of a TREM using chemical synthesis.

The TREM, in this example, tRNA-Thr-CGT, is chemically synthesized with the sequence GGCUCUAUGGCUUAGUUGGUUAAAGCGCCUGUCUCGUAAACAGGAGAUCCUGGG UUCGACUCCCAGUGGGGCCUCAA. This TREM is produced by solid-phase chemical synthesis using phosphoroamedite chemistry as previously described, for example as in Zlatev et. al. (2012) Current Protocols, 50 (1), 1.28.1-1.28.16. Briefly, protected RNA phorphoroamedites are sequentially added in a desired order to a growing chain immobilized on a solid support (e.g. controlled pore glass). Each cycle of addition has multiple steps, including: (i) deblocking the DMT group protecting the 5′-hydroxyl of the growing chain, (ii) coupling the growing chain to an incoming phosphoramidite building block, (iii) capping any chain molecules still featuring a 5′-hydroxyl, i.e. those that failed to couple with the desired incoming building block, and (iv) oxidation of the newly formed tricoordinated phosphite triester linkage. After the final building block has been coupled and oxidized, the chain is cleaved from the solid support and all protecting groups except for the DMT group protecting the 5′-hydroxyl are removed. The chain is then purified by RP-HPLC (e.g., DMT-on purification) and the fraction containing the chain is subjected to deprotection of the DMT group under acidic conditions, affording the final TREM. The TREM will feature a 5′-phosphate and a 3′-OH. The TREM is then admixed with a pharmaceutically acceptable excipient to make a test TREM product.

If the TREM needs to be charged, the TREM produced by the chemical synthesis reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with threonyl-tRNA synthetase and threonine, respectively, and then phenol extracted, filtered using a Nuc-trap column, and ethanol precipitated. The TREM is then admixed with a pharmaceutically acceptable excipient to make a test TREM product.

### Example 41: Production of a Candidate TREM Complementary to the SMC Through In Vitro Transcription

This example describes production of a TREM using in vitro transcription (IVT).

The TREM, in this example, tRNA-Leu-CAA, is produced using in vitro transcription with the sequence GUCAGGAUGGCCGAGUGGUCUAAGGCGCCAGACUCAAGUUCUGGUCUCCGUAUG GAGGCGUGGGUUCGAAUCCCACUUCUGACA as previously described in Pestova et al., RNA 7(10):1496-505 (2001). Briefly, a DNA plasmid containing a bacteriophage T7 promoter followed by the tRNA-Leu-CAA gene sequence is linearized and transcribed in vitro with T7 RNA polymerase at 37° C. for 45 min and then phenol extracted, filtered using a Nuc-trap column, and ethanol precipitated. The TREM is then admixed with a pharmaceutically acceptable excipient to make a test TREM product.

If the TREM needs to be charged, the TREM produced by the IVT reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with leucyl-tRNA synthetase and leucine, respectively, and then phenol extracted, filtered using a Nuc-trap column, and ethanol precipitated. The TREM is then admixed with a pharmaceutically acceptable excipient to make a test TREM product.

### Example 42: Modulation of a tRNA Pool Through TREM Administration to a Cell

This example describes administration of a TREM to a cell to modulate tRNA pools in the cell.

TREMs produced as in Examples 38-41 are delivered to a cell through electroporation, as previously described in Nature Methods 3, 67-68 (2006). Briefly, 10⁶-10⁷ cells, in this example the human epithelial MCF10A cells, are transferred in an electroporation cuvette and mixed gently after the addition of 1-30 ug of TREM, in this example tRNA-Thr-CGT with the sequence GGCUCUAUGGCUUAGUUGGUUAAAGCGCCUGUCUCGUAAACAGGAGAUCCUGGG UUCGACUCCCAGUGGGGCCUCAA. The cuvette is transferred to the electroporator and the device is discharged (a voltage of 200-350V is used). Place cuvette on ice and transfer the electroporated cells to a culture dish with complete medium and transfer to an incubator for 24-48 hrs.

Once delivered, the change in tRNA pools can be quantified by methods such as Nanopore sequencing, tRNA-sequencing, Northern blotting or quantitative RT-PCR. In this example, the tRNA pool changes are monitored using Oxford Nanopore direct RNA sequencing, as previously described in Sadaoka et al., Nature Communications (2019) 10, 754.

Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 30 minutes. The solution is neutralized by the addition of an equal volume of 100 mM Na-acetate/acetic acid (pH 4.8) and 100 mM NaCl, followed by ethanol precipitation. Deacylated sRNA is dissolved in water, and its integrity verified by agarose gel electrophoresis. Deacylated sRNA is then polyadenylated using yeast poly(A) tailing kit per manufacturer's instructions to generate a sRNA polyadenylated pool. Following polyadenylation, a reverse transcription reaction is performed to generate cDNA using SuperScript III Reverse Transcriptase (Thermo Fisher Scientific) or a thermostable group II intron RT (TGIRT, InGex LLC) that is less sensitive to RNA structure and modifications. A sequencing adapter is ligated onto the cDNA mixture by incubating the cDNA mixture with RNA adapter, T4 ligase and ligation buffer following the standard protocol for Oxford Nanopore. Nanopore sequencing is then performed on the libraries and the sequences are mapped to a genomic database, in this example to the genomic tRNA database, GtRNAdb. The methods described in this example can be adopted for use to evaluate the tRNA pool in the cells administered with a TREM compared to those not administered with a TREM.

### Example 43: Modulation of a tRNA Pool Through TREM Administration to a Cell Using Liposome

This example describes administration of a TREM to a cell using liposome vesicles to modulate tRNA pools in the cell.

TREMs produced as in Examples 38-41 are delivered to a cell in a vesicle or other lipid-based carrier, such as liposomes or lipid nanoparticles. In this example, a liposome kit (from Sigma or other vendor) is used to prepare liposomes containing the TREM, in this example tRNA-Thr-CGT with the sequence GGCUCUAUGGCUUAGUUGGUUAAAGCGCCUGUCUCGUAAACAGGAGAUCCUGGG UUCGACUCCCAGUGGGGCCUCAA following manufacturer's directions. The human cell line, HEK293T, is used in this example. Cells are seeded to obtain 70-80% confluency the day of the transfection. The media is replaced 30 minutes prior to the transfection with serum-free media after which the liposomes are added to the cell media.

Once delivered, the change in tRNA pools can be quantified by methods such as Nanopore sequencing, tRNA-sequencing (Zheng et al., Nature Methods 12, 835-837 (2015)), Northern blotting or quantitative RT-PCR. In this example, the tRNA pool changes are monitored using tRNA-sequencing. Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is treated with a demethylase mixture to remove m¹A, m¹G and m³C modifications located at the Watson-Crick face. Following demethylation of the tRNA pool, a cDNA library is generated from the tRNAs using a thermostable group II intron RT (TGIRT) that is less sensitive to tRNA structure. This reverse transcriptase adds RNA-sequencing adaptors to the tRNAs by template-switching without requiring RNA ligation. Illumina sequencing is then performed on the libraries generated from the tRNAs and the sequencing reads are mapped to a genomic database, in this example to the genomic tRNA database, GtRNAdb. The methods described in this example can be adopted for use to evaluate the tRNA pool in the cells administered with a TREM compared to those not administered with a TREM.

### Example 44: Modulation of a tRNA Pool Through Delivery of TREM-Encoding Plasmid to a Cell

This example describes delivery of TREM-encoding plasmid to a cell to modulate tRNA pools in the cell.

A TREM is expressed in cells through delivery of a TREM-encoding plasmid using a vesicle-based carrier. To express a TREM in human cells, a plasmid is created, which contains a tRNA gene, in this example, tRNA-Gly-GCC, with the sequence GCATTGGTGGTTCAGTGGTAGAATTCTCGCCTGCCACGCGGGAGGCCCGGGTTCGAT TCCCGGCCAATGCA. The plasmid is generated using seamless assembly of DNA fragments, in this example using NEBuilder HiFi Assembly Master Mix, where a linearized mammalian expression vector of interest, in this example pLKO.1-puro-turboGFP linearized by PpuMI enzyme restriction, is fused with a DNA fragment that contains the tRNA gene. The DNA fragment in this example includes the following elements in 5′ to 3′ order: a 25 nucleotide-long sequence from the 3′ end of the vector linearization site, a U6 promoter, the tRNA sequence, a RNA polymerase III termination signal, a 25 nucleotide-long sequence from the 5′ end of the vector linearization site.

Once the plasmid is made, the human cell line, in this example HEK293T, is transfected with TREM-encoding plasmid, using Lipofactamine 3000 following manufacturer's directions. Once delivered, the change in tRNA pools can be quantified by methods such as Nanopore sequencing, tRNA-sequencing (Zheng et al., Nature Methods 12, 835-837 (2015)), Northern blotting or quantitative RT-PCR. In this example, the tRNA pool changes are monitored using tRNA-sequencing. Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is treated with a demethylase mixture to remove m¹A, m¹G and m³C modifications located at the Watson-Crick face. Following demethylation of the tRNA pool, a cDNA library is generated from the tRNAs using a thermostable group II intron RT (TGIRT) that is less sensitive to tRNA structure. This reverse transcriptase adds RNA-sequencing adaptors to the tRNAs by template-switching without requiring RNA ligation. Illumina sequencing is then performed on the libraries generated from the tRNAs and the sequencing reads are mapped to a genomic database, in this example to the genomic tRNA database, GtRNAdb. The methods described in this example can be adopted for use to evaluate the tRNA pool in the cells administered with a TREM compared to those not administered with a TREM.

### Example 45: Modulation of a tRNA Pool Through Delivery of a TREM-Encoding Viral Vector to a Cell

This example describes delivery of a TREM-encoding viral vector to a cell to modulate tRNA pools in the cell.

A TREM is expressed in cells through delivery of a TREM-encoding viral vector. In this example, a lentivirus packaging and delivery system encoding a TREM is used. Briefly, the TREM-encoding viral vector is built by first generating a plasmid comprising a TREM, in this example, tRNA-Gly-GCC, with the sequence GCATTGGTGGTTCAGTGGTAGAATTCTCGCCTGCCACGCGGGAGGCCCGGGTTCGAT TCCCGGCCAATGCA. The plasmid is generated using seamless assembly of DNA fragments where the pLKO.1-puro-turboGFP linearized vector is ligated to a DNA fragment containing the tRNA sequence as described in Example 25. To prepare a TREM expressing lentivirus, HEK293T cells are co-transfected with 3 μg of each packaging vector (pRSV-Rev, pCMV-VSVG-G and pCgpV) and 9 μg of the plasmid comprising a TREM, using Lipofectamine 3000 according to manufacturer's instructions. After 24 hours, the media is replaced with fresh antibiotic-free media and after 48 hours, virus-containing supernatant is collected and centrifuged for 10 min at 2000 rpm before being filtered through a 0.45 m filter.

The cell of interest is then infected with the virus. In this example, 2 mL of virus prepared is used to transduce 100,000 HeLa cells, in the presence of 8 μg/mL polybrene. Forty-eight hours after transduction, puromycin (at 2 μg/mL) antibiotic selection is performed for 2-7 days alongside a population of untransduced control cells to select for cells that integrated the TREM in their genome for expression.

The change in tRNA pools can be quantified by methods such as Nanopore sequencing, tRNA-sequencing (Zheng et al., Nature Methods 12, 835-837 (2015)), Northern blotting or quantitative RT-PCR. In this example, the tRNA pool changes are monitored using quantitative RT-PCR (Korniy et al., Nucleic Acids Research (2019), gkz202). Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is treated with a demethylase mixture to remove m¹A, m¹G and m³C modifications located at the Watson-Crick face. Following demethylation, the pool is reverse transcribed into cDNA using stem-loop adapters complimentary to the 3′-ends of the tRNAs of interest. In this step, reverse transcription (RT) is performed using the Superscript III first strand synthesis system (ThermoFisher Scientific). Quantitative PCR is then performed using the QuantiTect SYBR Green Kit (Qiagen) according to the manufacturer's protocol with forward primers complimentary to the region of cDNA encoded by the tRNAs of interest and a universal primer complimentary to the stem-loop adapter appended during RT. The methods described in this example can be adopted for use to evaluate the levels of glycine specifying molecule that can pair with the CGT codon in the cells administered with a TREM compared to those not administered with a TREM.

### Example 46: System to Test the Effects of TREM Administration on an SMC Containing ORF

This example describes a system, in this example a cell line, that expresses an SMC-containing ORF to study the effects of TREM administration.

To study the effects of TREM administration on a SMC-containing ORF, in this example on the rs2229882 polymorphism of the MAP3K1 gene, an established cell line, in this example human breast epithelial cells, such as MCF10A or 184A1 cells, are genomically edited by CRISPR-Cas to knock out the expression of the endogenous gene of interest, in this example the MAP3K1 gene. MAP3K1 knockout cells were generated using the CRISPR-Cas9 system to insert 1 bp in a coding exon of MAP3K1 to cause a frameshift mutation as previously described (for example, Bauer et al., J. Vis. Exp., (95), doi:10.3791/52118 (2015)). Briefly, an online design tool that predicts the most effective guide RNA to use for genome editing, for example, https://portals.broadinstitute.org/gpp/public/analysis-tools/sgrna-design, is used to select a high-score guide RNA (gRNA) containing a 20-base pair (bp) target sequence that minimizes genomic matches to reduce the risk of off-target site cleavage. In this example, the targeting sequence is CAGTGTGTGAAGACGGCTGC. The targeting sequence is cloned into pSpCas9 (BB) plasmid (pX330) (Addgene plasmid ID 42230). HEK293T cells are transiently transfected with the CRISPR/Cas9 construct targeting MAP3K1 and a puromycin expression construct for clone selection. The next day, cells are selected with puromycin for 2 days and subcloned to form single colonies. MAP3K1 KO clones are identified by PCR screen. The obtained clones are validated by qPCR and immunoblot using an antibody against MAP3K1.

Once created, this cell line is used to overexpress the WT or SMC-containing mRNA through transient plasmid transfection or through stable lentivirus transduction methods. The TREM of interest is then administered to each cell line and its effect on the SMC-containing ORF compared to the WT ORF is assessed using assays such as the ones described in Examples 25, 30 or 31.

### Example 47: Evaluation of Protein Expression Levels of SMC-Containing ORF with Administration of a TREM

This example describes administration of a TREM to alter expression levels of an SMC-containing ORF.

To create a system in which to study the effects of TREM administration on protein expression levels of an SMC-containing protein, in this example, from the PNPL3A gene coding for adiponutrin, a plasmid containing the PNPL3A rs738408 ORF sequence is transfected in the normal human hepatocyte cell line THLE-3, edited by CRISPR/Cas to contain a frameshift mutation in a coding exon of PNPLA3 to knock out endogenous PNPLA3 (THLE-3_PNPLA3KO cells). As a control, an aliquot of THLE-3_PNPLA3KO cells are transfected with a plasmid containing the wildtype PNPL3A ORF sequence.

### Synthesis and Preparation of TREM

An arginine TREM is synthesized as described in Examples 3-7 and quality control methods as described in Examples 8-10 are performed. To ensure proper folding, the TREM is heated at 85° C. for 2 minutes and then snap cooled at 4° C. for 5 minutes.

### Evaluation of Protein Level of SMC-Containing ORF

A TREM is delivered to the THLE-3_PNPLA3KO cells containing the rs738408 ORF sequence as well as to the THLE-3_PNPLA3KO cells containing the wildtype PNPL3A ORF sequence. In this example, the TREM contains a proline isoacceptor containing an AGG anticodon, that base pairs to the CCT codon, i.e. with the sequence GGCUCGUUGGUCUAGGGGUAUGAUUCUCGCUUAGGGUGCGAGAGGUCCCGGGUU CAAAUCCCGGACGAGCCC. A time course is performed ranging from 30 minutes to 6 hours with hour-long interval time points. At each time point, cells are trypsinized, washed and lysed. Cell lysates are analyzed by Western blotting and blots are probed with antibodies against the adiponutrin protein. A total protein loading control, such as GAPDH, actin or tubulin, is also probed as a loading control.

The methods described in this example can be adopted for use to evaluate the expression levels of the adiponutrin protein in rs738408 ORF containing cells.

### Example 48: Modulation of Protein Translation Rate of SMC-Containing ORF with TREM Administration

This example describes administration of a TREM to alter the rate of protein translation of an SMC-containing ORF.

To monitor the effects of TREM addition on translation elongation rates, an in vitro translation system, in this example the RRL system from Promega, is used in which the fluorescence change over time of a reporter gene, in this example GFP, is a surrogate for translation rates.

### Synthesis and Preparation of TREM

An arginine TREM is synthesized as described in Examples 3-7 and quality control methods as described in Examples 8-10 are performed. To ensure proper folding, the TREM is heated at 85° C. for 2 minutes and then snap cooled at 4° C. for 5 minutes.

Evaluation of protein translation rate of SMC-containing ORF First, a rabbit reticulocyte lysate that is depleted of the endogenous tRNA using an antisense oligonucleotide targeting the sequence between the anticodon and variable loop is generated (see, e.g., Cui et al. 2018. Nucleic Acids Res. 46(12):6387-6400). In this example, a TREM comprising an alanine isoacceptor containing an UGC anticodon, that base pairs to the GCA codon, i.e. with the sequence GGGGAUGUAGCUCAGUGGUAGAGCGCAUGCUUUGCAUGUAUGAGGUCCCGGGUU CGAUCCCCGGCAUCUCCA is added to the in vitro translation assay lysate in addition to 0.1-0.5 ug/uL of mRNA coding for the wildtype TERT ORF fused to the GFP ORF by a linker or an mRNA coding for the rs2736098 TERT ORF fused to the GFP ORF by a linker. The progress of GFP mRNA translation is monitored by fluorescence increase on a microplate reader at 37° C. using λex485/λem528 with data points collected every 30 seconds over a period of 1 hour. The amount of fluorescence change over time is plotted to determine the rate of translation elongation of the wildtype ORF compared to the rs2736098 ORF with and without TREM addition. The methods described in this example can be adopted for use to evaluate the translation rate of the rs2736098 ORF and the wildtype ORF in the presence or absence of TREM.

### Example 49: Determining that Administration of a TREM Affects Protein Expression Levels of SMC-Containing ORF

This example describes administration of a TREM to alter expression levels of an SMC-containing ORF.

To create a system in which to study the effects of TREM administration on protein expression levels of SMC-containing protein, in this example, from the PNPL3A gene coding for adiponutrin, a plasmid containing the PNPL3A rs738408 ORF sequence is transfected in the normal human hepatocyte cell line THLE-3, edited by CRISPR/Cas to contain a frameshift mutation in a coding exon of PNPLA3 to knock out endogenous PNPLA3 (THLE-3_PNPLA3KO cells). As a control, an aliquot of THLE-3_PNPLA3KO cells are transfected with a plasmid containing the wildtype PNPL3A ORF sequence.

A TREM is delivered to the THLE-3_PNPLA3KO cells containing the rs738408 ORF sequence as well as to the THLE-3_PNPLA3KO cells containing the wildtype PNPL3A ORF sequence. In this example, the TREM contains a proline isoacceptor containing an AGG anticodon, that base pairs to the CCT codon, i.e. with the sequence GGCUCGUUGGUCUAGGGGUAUGAUUCUCGCUUAGGGUGCGAGAGGUCCCGGGUU CAAAUCCCGGACGAGCCC. A time course is performed ranging from 30 minutes to 6 hours with hour-long interval time points. At each time point, cells are trypsinized, washed and lysed. Cell lysates are analyzed by Western blotting and blots are probed with antibodies against the adiponutrin protein. A total protein loading control, such as GAPDH, actin or tubulin, is also probed as a loading control.

The methods described in this example can be adopted for use to evaluate the expression levels of the adiponutrin protein in rs738408 ORF containing cells.

### Example 50: TREM Administration to Change Protein Translation Rate of SMC-Containing ORF

This example describes administration of a TREM to alter the rate of protein translation of an SMC-containing ORF.

To monitor the effects of TREM addition on translation elongation rates, an in vitro translation system, in this example the RRL system from Promega, is used in which the fluorescence change over time of a reporter gene, in this example GFP, is a surrogate for translation rates. First, a rabbit reticulocyte lysate that is depleted of the endogenous tRNA using an antisense oligonucleotide targeting the sequence between the anticodon and variable loop is generated (see, e.g., Cui et al. 2018. Nucleic Acids Res. 46(12):6387-6400). In this example, a TREM comprising an alanine isoacceptor containing an UGC anticodon, that base pairs to the GCA codon, i.e. with the sequence GGGGAUGUAGCUCAGUGGUAGAGCGCAUGCUUUGCAUGUAUGAGGUCCCGGGUU CGAUCCCCGGCAUCUCCA is added to the in vitro translation assay lysate in addition to 0.1-0.5 ug/uL of mRNA coding for the wildtype TERT ORF fused to the GFP ORF by a linker or an mRNA coding for the rs2736098 TERT ORF fused to the GFP ORF by a linker. The progress of GFP mRNA translation is monitored by fluorescence increase on a microplate reader at 37° C. using λex485/λem528 with data points collected every 30 seconds over a period of 1 hour. The amount of fluorescence change over time is plotted to determine the rate of translation elongation of the wildtype ORF compared to the rs2736098 ORF with and without TREM addition. The methods described in this example can be adopted for use to evaluate the translation rate of the rs2736098 ORF and the wildtype ORF in the presence or absence of TREM.

### Example 51: Determining that Modulation of a TREM Complementary to the SMC Changes the Function of the Protein Derived from the SMC-Containing ORF

This example describes administration of a TREM to change the function of an SMC-containing ORF.

Using an in vitro translation (IVT) system (such as the RRL system from Promega), the wildtype and SMC containing mRNAs are translated in the presence and absence of a TREM. In this example the SMC-containing gene is AChE, coding for the acetylcholinesterase protein, and the TREM contains a proline isoacceptor containing an AGG anticodon, that base pairs to the CCU codon, i.e. with the sequence GGCUCGUUGGUCUAGGGGUAUGAUCUCGCUUAGGGUGCGAGAGGUCCCGGGUUC AAAUCCCGGACGAGCCC.

To determine if addition of the TREM changes the functional activity of the SMC-containing protein, in this example the acetylcholinesterase protein, a functional assay that uses DTNB to quantify the thiocholine produced from the hydrolysis of acetylthiocholine by AChE is used. Briefly, the translation reactions are incubated at room temperature for 10-30 minutes with the kit AChE reaction mixture, after which the absorption intensity of DTNB adduct at OD 410 nm is used to measure the amount of thiocholine formed, which is proportional to the AChE activity. The methods described in this example can be adopted for use to evaluate AChE activity of the protein resulting from the translation of the rs7636 AChE mRNA or the wildtype AChE mRNA.

### Example 52: Determining that Modulation of a TREM Complementary to an SMC Changes the Localization of the Protein Derived from the SMC-Containing ORF

This example describes administration of a TREM to alter the localization of an SMC-containing ORF.

To create a system in which to study the effects of TREM administration on protein localization of an SMC-containing ORF, a plasmid containing the CFTR rs1042077 ORF sequence tagged with a reporter, such as GFP or myc, is transfected in the human lung epithelial cell line MRC-5. As a control, a plasmid containing the wildtype CFTR ORF sequence tagged with a reporter is also transfected in parallel in MRC-5 cells.

To determine if TREM addition changes the localization of CFTR, the cells are seeded on coverslips and 24 hours later are transfected with a TREM complementary to the CFTR SMC or a control TREM. In this example the TREM complementary to the CFTR SMC comprises a threonine isoacceptor containing an CGU anticodon, that base pairs to the ACG codon, i.e. with the sequence GGCUCUGUGGCUUAGUUGGCUAAAGCGCCUGUCUCGUAAACAGGAGAUCCUGGG UUCGAAUCCCAGCGGGGCCU. The control TREM consists of either a scrambled sequence or the threonine sequence where the 5′ end of the TREM has been changed to prevent charging. After 24 hours, the cells are fixed, stained for CFTR and its reporter and visualized under a microscope. The methods described in this example can be adopted for use to evaluate the localization of wildtype CFTR and rs1042077 CFTR.

### Example 53: Determining that Modulation of a TREM Complementary to an SMC Changes Folding of the Protein Translated from the SMC-Containing ORF

This example describes administration of a TREM to alter the folding of an SMC-containing ORF.

### Plasmid Generation and Transfection

To identify SMCs that result in protein misfolding, the SMC-ORF containing protein, in this example the rs7190458 BCAR1 ORF is synthesized and cloned into a plasmid containing a CMV promoter (or any other mammalian promoter) and a purification tag, in this example a FLAG tag (DYKDDDDK epitope), following the manufacturer's instructions and standard molecular cloning techniques. Here, the pFLAG-CMV-1 plasmid is used. The plasmid is transfected in the human HeLa cell line. A TREM, in this example comprising a leucine isoacceptor containing an UUG anticodon, that base pairs to the CUU codon, i.e. with the sequence GGUAGCGUGGCCGAGCGGUCUAAGGCGCUGGAUUAAGGCUCCAGUCUCUUCGGA GGCGUGGGUUCGAAUCCCACCGCUGCCA is also transfected into the HeLa cells. As a control, the BCAR1 KO cells are transfected with the SMC BCAR1 ORF containing plasmid alone and separately with a plasmid containing the wildtype BCAR1 ORF sequence.

### Purification

At the optimized harvest timepoint, in this example 72 hours post-transfection, the cells are lysed, and centrifuged at 12,000×g for 10 minutes. The supernatant is loaded under gravity flow onto a pre-packed and equilibrated anti-flag packed M2-agarose column. The column is washed with 10-20 column volumes of TBS (Tris HCl, NaCl) or with a salt containing buffer. To elute the FLAG-tagged protein from the beads, the beads are incubated with FLAG-tag peptide. The eluate is run on an SDS-PAGE gel for purity quality control. This purification is performed on cells transfected with the WT BCAR1 ORF and the SMC BCAR1 ORF in the presence and absence of a TREM.

### Initial Examination of Protein Folding

To examine the effects of protein folding, the stability of the purified proteins derived from the WT and SMC containing ORFs are monitored using thermal melting. In this example, Differential Scanning Fluorimetry (DSF) with a fluorescent dye (Sypro Orange), which measures the changes of binding of the intercalator dye to the unfolding protein is used. Alterations in protein folding results in variations of the thermal melting curves. Using this methodology, the SMC ORF-derived protein with and without TREM addition is compared to the control wildtype BCAR1. The methods described in this example can be adopted for use to evaluate the thermal melting curve of proteins derived from SMC-containing ORFs.

### Example 54: Determining that Modulation of a TREM Complementary to an SMC Alters the Cellular Phenotype Resulting from Translation of the SMC-Containing ORF

This example describes administration of a TREM to alter the cellular phenotype of an SMC-containing ORF.

To create a system in which to study the effects of TREM administration on cellular processes, in this example on cell migration, a plasmid containing the SMC-containing ORF, in this example the rs7190458 BCAR1 ORF sequence, is transfected in the human pancreatic cancer cell line PANC-1, in which BCAR1 has been knocked out using CRISPR/Cas. As a control, the PANC-1 BCAR1 KO cells are transfected with a plasmid containing the WT BCAR1 ORF sequence.

A TREM, in this example comprising a leucine isoacceptor containing an UUG anticodon, that base pairs to the CUU codon, i.e. with the sequence GGUAGCGUGGCCGAGCGGUCUAAGGCGCUGGAUUAAGGCUCCAGUCUCUUCGGA GGCGUGGGUUCGAAUCCCACCGCUGCCA is delivered to the PANC-1 cells. Delivery of a control TREM containing either a scrambled sequence or the leucine sequence where the 5′ end of the TREM has been changed to prevent charging are used as a control. The cells are grown to 80% confluency in a monolayer and scratched with a new 1 ml pipette tip across the center of the well. The cells are rinsed twice to remove floating cells and the media is replenished. After 48 hours, the cells are fixed and stained with crystal violet. The stained monolayer is photographed, and the gap distance quantified. The methods described in this example can be adopted for use to evaluate the migratory phenotype of cells administered a TREM.

### Example 55: Modulation of TREM to Ameliorate a Disease State Resulting from Translation of the SMC-Containing ORF

This example describes increasing TREM levels to ameliorate a disease state resulting from an SMC-containing ORF.

To create a system in which to study the effects of TREM administration on disease state, in this example on breast cancer onset, a plasmid containing the SMC-containing ORF, in this example the rs2229882 MAP3K1 ORF sequence, is transfected in the human non-transformed breast cell line MCF10A, in which MAP3K1 has been knocked out using CRISPR/Cas. As a control, the MCF10A MAP3K1 KO cells are transfected with a plasmid containing the wildtype MAP3K1 ORF sequence.

A TREM, in this example comprising a threonine isoacceptor containing an AGU anticodon, that base pairs to the ACU codon, i.e. with the sequence GGCGCCGUGGCUUAGUUGGUUAAAGCGCCUGUCUAGUAAACAGGAGAUCCUGGG UUCGAAUCCCAGCGGUGCCU is delivered to the MCF10A cells. Delivery of a control TREM containing either a scrambled sequence or the threonine sequence where the 5′ end of the TREM has been changed to prevent charging are used as a control. The cells are monitored for increased MAPK signaling by Western blotting using antibodies against the phosphorylation state of the ERK and JNK kinases. A total protein loading control, such as GAPDH, actin or tubulin, as well as the non-modified (i.e. non-phosphorylated) signaling protein, i.e. using ERK as a control for phospho-ERK, are probed as loading controls. The cells are also monitored for cell proliferation and invasion using standard proliferation and transwell invasion assays. To monitor breast cancer progression, the cells are injected subcutaneously or in the mammary fat pad of SCID mice and tumor volume is monitored daily using calipers to measure the length, width and height of the tumor(s). The methods described in this example can be adopted for use to evaluate tumor phenotype.

What is claimed is:

1. A method of evaluating a tRNA pool in a cell or subject comprising an endogenous open reading frame (ORF), which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the cell or subject wherein:
(i) is a tRNA moiety having an anticodon that pairs with the codon of the ORF having a first sequence (the first tRNA moiety); and
(ii) is an isoacceptor tRNA moiety having an anticodon that pairs with a codon other than the codon having the first sequence (the second tRNA moiety) in the cell or subject,
thereby evaluating the tRNA pool in the cell or subject.

2. The method of claim 1, comprising acquiring knowledge of (i).

3. The method of claim 1, comprising acquiring knowledge of (ii).

4. The method of claim 1, comprising acquiring knowledge of (i) and (ii).

5. The method of any one of claims 1-4, wherein:
acquiring knowledge of (i) comprises acquiring a value for the abundance, e.g., relative amount, of (i); and/or
acquiring knowledge of (ii) comprises acquiring a value for the abundance, e.g., relative amount, of (ii).

6. The method of claim 5, wherein responsive to said value, the method comprises administering a TREM composition comprising a TREM, a TREM core fragment, or a TREM fragment described herein, wherein the TREM, TREM core fragment or TREM fragment has an anticodon that pairs with: (a) the codon having the first sequence; or (b) the codon other than the codon having the first sequence, in an amount and for a time sufficient to modulate the relative amounts of the first tRNA moiety and the second tRNA moiety.

7. A method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;
contacting the cell with a TREM composition comprising a TREM, a TREM core fragment, or a TREM fragment described herein, wherein the TREM, TREM core fragment or TREM fragment has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the cell,
thereby modulating the tRNA pool in the cell.

8. The method of claim 7, wherein the TREM comprises an anticodon that pairs with (a).

9. The method of claim 7, wherein the TREM comprises an anticodon that pairs with (b).

10. A method of modulating a tRNA pool in a subject having an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;
contacting the subject with a TREM composition comprising a TREM, a TREM core fragment, or a TREM fragment described herein, wherein the TREM, TREM core fragment or TREM fragment has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the subject,
thereby modulating the tRNA pool in the subject.

11. The method of claim 10, wherein the TREM comprises an anticodon that pairs with (a).

12. The method of claim 10, wherein the TREM comprises an anticodon that pairs with (b).

13. The method of any one of claims 10-12, comprising acquiring knowledge of (i).

14. The method of any one of claims 10-12, comprising acquiring knowledge of (ii).

15. The method of any one of claims 10-12, comprising acquiring knowledge of (i) and (ii).

16. The method of any one of claims 13-15, wherein:
acquiring knowledge of (i) comprises acquiring a value for the abundance, e.g., relative amounts, of (i); and/or
acquiring knowledge of (ii) comprises acquiring a value for the abundance, e.g., relative amounts, of (ii).

17. The method of claim 16, wherein responsive to said value, the cell or subject is contacted with the TREM composition having an anticodon that pairs with (a) or (b).

18. A method of modulating a tRNA pool in a subject having an endogenous open reading frame (ORF) comprising a codon comprising a synonymous mutation (a synonymous mutation codon or SMC), comprising:
providing a TREM composition comprising a TREM, a TREM core fragment, or a TREM fragment described herein, wherein the TREM, TREM core fragment or TREM fragment comprises an isoacceptor tRNA moiety comprising an anticodon sequence that pairs with the SMC (the TREM);
contacting the subject with the TREM composition in an amount and/or for a time sufficient to modulate the tRNA pool in the subject,
thereby modulating the tRNA pool in the subject.

19. A method of modulating a tRNA pool in a cell comprising an endogenous open reading frame (ORF) comprising a codon comprising a synonymous mutation (a synonymous mutation codon or SMC), comprising:
providing a TREM composition comprising a TREM, a TREM core fragment, or a TREM fragment described herein, wherein the TREM, TREM core fragment or TREM fragment comprises an isoacceptor tRNA moiety comprising an anticodon sequence that pairs with the SMC (the TREM);
contacting the cell with the TREM composition in an amount and/or for a time sufficient to modulate the tRNA pool in the cell,
thereby modulating the tRNA pool in the cell.

20. The method of claim 19 or 18, comprising acquiring knowledge of the abundance of one or both of (i) and (ii) e.g., acquiring knowledge of the relative amounts of (i) and (ii) wherein (i) is a tRNA moiety having an anticodon that pairs with the SMC (the first tRNA moiety) and (ii) is an isoacceptor tRNA moiety having an anticodon that pairs with a codon other than the SMC (the second tRNA moiety), in the subject or cell.

21. The method of claim 20, comprising acquiring knowledge of (i).

22. The method of claim 20, comprising acquiring knowledge of (ii).

23. The method of claim 20, comprising acquiring knowledge of (i) and (ii).

24. The method of claim 20, wherein acquiring knowledge of (i) comprises acquiring a value for the abundance, e.g., relative amounts, of (i).

25. The method of claim 20, wherein acquiring knowledge of (ii) comprises acquiring a value for the abundance, e.g., relative amounts, of (ii).

26. The method of claim 24 or 25, wherein responsive to said value, the cell or subject is contacted with the TREM composition.

27. A method of treating a subject having an endogenous open reading frame (ORF) comprising a codon having a first sequence, wherein:
(i) acquiring, e.g., directly or indirectly acquiring, a value for the status of the codon having the first sequence in the subject, wherein said value comprises a measure of the presence or absence of the codon having the first sequence in a sample from the subject; and identifying the subject as having the codon having the first sequence; and
(ii) responsive to said value, administering a TREM composition comprising a TREM, a TREM core fragment, or a TREM fragment described herein, wherein the TREM, TREM core fragment or TREM fragment comprises an isoacceptor tRNA moiety having an anticodon that pairs with the codon having the first sequence, to the subject,
thereby treating the subject.

28. A method of treating a subject having an endogenous open reading frame (ORF) comprising a codon comprising a synonymous mutation (a synonymous mutation codon or SMC), comprising:
(i) acquiring, e.g., directly or indirectly acquiring, a value for the SMC status of the subject, wherein said value comprises a measure of the presence or absence of SMC in a sample from the subject, and identifying the subject as having a SMC; and
(ii) responsive to said value, administering a TREM composition comprising a TREM, a TREM core fragment, or a TREM fragment described herein, wherein the TREM, TREM core fragment or TREM fragment comprises comprising an isoacceptor tRNA moiety having an anticodon that pairs with the SMC, to the subject,
thereby treating the subject.

29. A method of evaluating a subject having an endogenous open reading frame (ORF) comprising a codon having a first sequence, comprising:
acquiring, e.g., directly or indirectly acquiring, a value for the status of the codon having the first sequence in the subject, wherein said value comprises a measure of the presence or absence of the codon having the first sequence in a sample from the subject; and
identifying the subject as having a codon having the first sequence,
thereby evaluating the subject.

30. A method of evaluating a subject having an endogenous open reading frame (ORF) comprising a codon comprising a synonymous mutation (a synonymous mutation codon or SMC), comprising:
acquiring, e.g., directly or indirectly acquiring, a value for the SMC status of the subject, wherein said value comprises a measure of the presence or absence of SMC in a sample from the subject; and
identifying the subject as having a SMC,
thereby evaluating the subject.

31. The method of claim 29 or 30, wherein the TREM, TREM core fragment or TREM fragment does not comprise an anticodon that pairs with a stop codon.

32. The method of any one of claims 29-31, wherein: (a) the ORF codon having the first sequence; or (b) the SMC; is other than a stop codon, e.g., TAA, TGA or TAG.

33. The method of any one of claims 1-32, wherein: (a) the ORF codon having the first sequence; or (b) the SMC; in the absence of contact with the composition comprising a TREM, is associated with a phenotype, e.g., an unwanted phenotype, e.g., a disorder or symptom, e.g., a disorder or symptom chosen from Table 1 or FIG. 4.

34. The method of claim 32, wherein the disorder or symptom is chosen from a disease group provided in Table 1, e.g., cardiovascular, dermatology, endocrine, immunology, neurology, oncology, ophthalmology, or respiratory.

35. A method of modulating a production parameter of an RNA, or a protein encoded by an RNA, in a target cell or tissue, comprising:
providing, e.g., administering, to the target cell or tissue, or contacting the target cell or tissue with, an effective amount of a TREM composition comprising a TREM, a TREM core fragment, or a TREM fragment described herein, which TREM, TREM core fragment or TREM fragment corresponds to a contextually-rare codon (“con-rare codon”) of the RNA,
thereby modulating the production parameter of the RNA, or protein encoded by the RNA in the target cell or tissue.

36. The method of claim 35, wherein the target cell or tissue is obtained from a subject.

37. The method of claim 36, comprising administering the TREM composition to a subject.

38. The method of claim 37, comprising contacting the TREM composition with the target tissue or cell ex vivo.

39. The method of claim 38, comprising introducing the ex vivo-contacted target tissue or cell into a subject, e.g., an allogeneic or autologous subject.

40. The method of any one of claims 35-39, wherein the target cell or tissue is a specific or selected target cell or tissue, e.g., a cell or tissue type in a particular developmental stage; a cell or tissue type in a particular disease state; or a cell present in a particular extracellular milieu.

41. The method of any one of claims 35-40, wherein the production parameter comprises an expression parameter or a signaling parameter, e.g., as described herein.

42. The method of any one of claims 35-41, wherein the production parameter of the RNA is modulated, e.g., an RNA that can be translated into a polypeptide, e.g., a messenger RNA.

43. The method of claim 42, wherein the production parameter of the RNA is increased or decreased.

44. The method of any one of claims 35-43, wherein the production parameter of the protein encoded by the RNA is modulated.

45. The method of claim 44, wherein the production parameter of the protein is increased.

46. The method of claim 45, wherein the production parameter of the protein is decreased.

47. A method of determining the presence of a nucleic acid sequence, e.g., a DNA or RNA, having a contextually-rare codon (“con-rare codon nucleic acid sequence”), comprising:
acquiring knowledge of the presence of the con-rare codon nucleic acid sequence in a sample from a subject, e.g., a target cell or tissue sample,
wherein responsive to the acquisition of knowledge of the presence of the con-rare codon nucleic acid sequence:
(1) the subject is classified as being a candidate to receive administration of an effective amount of a TREM composition comprising a TREM, a TREM core fragment, or a TREM fragment described herein, which TREM, TREM core fragment or TREM fragment corresponds to a contextually-rare codon (“con-rare codon”) of the nucleic acid sequence; or
(2) the subject is identified as likely to respond to a treatment comprising the TREM composition.

48. A method of treating a subject having a disease associated with a contextually-rare codon (“con-rare codon”), comprising:
acquiring knowledge of the presence of a nucleic acid sequence, e.g., a DNA or RNA, having the con-rare codon (“con-rare codon nucleic acid sequence”) in a target cell or tissue sample from the subject; and
administering to the subject an effective amount of a TREM composition comprising a TREM, a TREM core fragment, or a TREM fragment described herein, which TREM, TREM core fragment or TREM fragment corresponds to the con-rare codon of the nucleic acid sequence,
thereby treating the disease in the subject.

49. A method of providing a tRNA effector molecule (TREM) to a subject, comprising:
providing, e.g., administering, to the subject, an effective amount of a TREM composition comprising a TREM, a TREM core fragment, or a TREM fragment described herein, which TREM, TREM core fragment or TREM fragment corresponds to a contextually-rare codon (“con-rare codon”) for a nucleic acid sequence in a target cell or tissue in the subject,
thereby providing a TREM composition to the subject.

50. A method of manufacturing a tRNA effector molecule (TREM) composition comprising:
identifying a TREM corresponding to a contextually-rare (con-rare) codon;
combining the TREM with a component, e.g., a carrier or excipient.
thereby manufacturing a TREM composition.

51. The method of any one of claims 35-50, wherein the method comprises acquiring a value for a con-rare codon in the nucleic acid sequence, e.g., DNA or RNA, wherein the value is a function of one or more of the following factors, e.g., by evaluating or determining one or more of the following factors:
(1) the sequence of the codon;
(2) the availability of a corresponding tRNA, e.g., charged tRNA, for that con-rare codon in a target cell or tissue, e.g., one or more iso-acceptor tRNA molecules;
(3) the expression profile (or proteomic properties) of the target cell or tissue (e.g., the abundance of expression of other proteins which include the con-rare codon);
(4) the proportion of the tRNAs corresponding to the con-rare codon which are charged; and
(5) the iso-decoder isotype of the tRNA corresponding to the con-rare codon.

52. The method of claim 51, wherein (1) comprises determining the presence or absence of a con-rare codon.

53. The method of claim 52, wherein a determination of the availability of a tRNA comprises acquiring a measure of one, two, three or all of the following parameters:
(a) level of a tRNA corresponding to the con-rare codon (“con-rare codon tRNA”) compared to a tRNA corresponding to a different codon;
(b) function, e.g., polypeptide chain elongation function, of a con-rare codon tRNA compared to a tRNA corresponding to a different codon;
(c) modification, e.g., aminoacylation or post-transcriptional modification, of a con-rare codon tRNA compared to a tRNA corresponding to a different codon; and/or
(d) sequence of a con-rare codon tRNA.

54. The method of claim 53, wherein a measure of availability (e.g., level) of a con-rare codon tRNA comprises a measure of the con-rare codon tRNA that is charged, e.g., aminoacylated, compared to: (1) the proportion of the con-rare codon tRNA that is not charged; or (2) the proportion of charged tRNA corresponding to a different codon.

55. The method of any one of claims 51-54, wherein responsive to said value, the target cell, or tissue, is identified as having a nucleic acid sequence having a con-rare codon (“con-rare codon nucleic acid sequence”) or an RNA having a con-rare codon (“con-rare codon RNA”).

56. The method of any one of claims 51-55, wherein responsive to said value, the RNA is identified as, an RNA having a con-rare codon.

57. The method of any one of claims 35-56, wherein modulation of a production parameter of the con-rare codon RNA comprises increasing a production parameter, e.g., an expression parameter or signaling parameter of the protein encoded by the con-rare codon RNA, e.g., increasing the expression level of the protein encoded by the con-rare codon RNA.

58. The method of any one of claims 35-57, wherein modulation of a production parameter of the con-rare codon RNA comprises decreasing a production parameter, e.g., an expression parameter or signaling parameter, of the protein encoded by the con-rare codon RNA, e.g., decreasing the expression level of the protein encoded by the con-rare codon RNA.

59. The method of any one of claims 51-54, wherein a determination of the expression profile (or proteome codon count) of the target cell or tissue, comprises a measure of:
(a) the abundance (e.g., expression) of proteins in a target cell or tissue; and
(b) a protein codon count for expressed proteins in a target cell or tissue.

60. The method of any one of claims 35-59, wherein the target cell or tissue is identified as comprising a con-rare-codon nucleic acid, e.g., RNA.

61. The method of any one of claims 35-60, wherein the con-rare codon meets a reference value for one or more of the following:
(1) the sequence of the codon;
(2) the availability of a corresponding tRNA, e.g., charged tRNA, for that con-rare codon in a target cell or tissue, e.g., one or more iso-acceptor tRNA molecules;
(3) the expression profile (or proteomic properties) of the target cell or tissue (e.g., the abundance of expression of other proteins which include the con-rare codon);
(4) the proportion of the tRNAs corresponding to the con-rare codon which are charged; and
(5) the iso-decoder isotype of the tRNA corresponding to the con-rare codon.

62. The method of any of claims 35-61, wherein at least 10%, 20%, 30%, 40%, 50%, 60%, 70%, 80%, 85%, 90%, 95%, 99%, or 100% (by weight or number) of the TREMs, TREM core fragments or TREM fragments in the TREM composition correspond to a con-rare codon.

63. The method of any one of claims 35-62, wherein the TREM composition comprises TREMs, TREM core fragments or TREM fragments that correspond to a plurality of con-rare codons.

64. The method of any of claims 35-63, wherein the TREM composition comprises: a first TREM which corresponds to a first con-rare codon; and an additional TREM which corresponds to a different con-rare codon.

65. The method of any of claims 35-64, wherein the TREM composition comprises: a first TREM which corresponds to a first con-rare codon; and a second TREM which corresponds to a second con-rare codon.

66. The method of any of claims 35-65, wherein at least 10%, 20%, 30%, 40%, 50%, 60%, 70%, 80%, 85%, 90%, 95%, 99%, or 100% (by weight or number) of the TREMs in the composition are charged.

67. The method of any of claims 35-66, wherein at least 10%, 20%, 30%, 40%, 50%, 60%, 70%, 80%, 85%, 90%, 95%, 99%, or 100% (by weight or number) of the TREMs in the composition are of the same iso-decoder isotype.

68. The method of any one of claims 35-67, wherein the cell is a host cell.

69. The method of any one of claims 35-68, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.

70. The method of any one of claims 35-69, wherein the cell comprises an exogenous nucleic acid sequence.

71. The method of any one of claims 35-71, wherein the cell is autologous to the exogenous nucleic acid sequence.

72. The method of any one of claims 35-71, wherein the cell is allogeneic to the exogenous nucleic acid sequence.

73. The method of any one of claims 35-72, wherein the exogenous nucleic acid sequence (e.g., DNA or RNA) comprises a con-rare codon.

74. The method of any one of claims 70-73, wherein administration of a TREM composition corresponding to the con-rare codon to the cell, modulates a production parameter, e.g., expression parameter or signaling parameter, of a product, e.g., RNA or polypeptide, of the exogenous nucleic sequence.

75. The method of any one of the preceding embodiments, wherein the TREM comprises a sequence of Formula A:
[L1]-[ASt Domain1]-[L2]-[DH Domain]-[L3]-[ACH Domain]-[VL Domain]-[TH Domain]-[L4]-[ASt Domain2],
wherein:
independently, [L1] and [VL Domain], are optional;
one of [L1], [ASt Domain1], [L2]-[DH Domain], [L3], [ACH Domain], [VL Domain], [TH Domain], [L4], and [ASt Domain2] comprises a nucleotide having a non-naturally occurring modification; and
wherein:
(a) the TREM retains the ability to: support protein synthesis, be charged by a synthetase, be bound by an elongation factor, introduce an amino acid into a peptide chain, support elongation, or support initiation;
(b) the TREM comprises at least X contiguous nucleotides without a non-naturally occurring modification, wherein X is greater than 10;
(c) at least 3, but less than all of the nucleotides of a type (e.g., A, T, C, G or U) comprise the same non-naturally occurring modification;
(d) at least X nucleotides of a type (e.g., A, T, C, G or U) do not comprise a non-naturally occurring modification, wherein X=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50;
(e) no more than 5, 10, or 15 nucleotides of a type (e.g., A, T, C, G or U) comprise a non-naturally occurring modification; and/or
(f) no more than 5, 10, or 15 nucleotides of a type (e.g., A, T, C, G or U) do not comprise a non-naturally occurring modification.

76. The method of claim 75, wherein the Domain comprising the non-naturally occurring modification retains a function, e.g., a domain function described herein.

77. The method of any one of the preceding embodiments, wherein the TREM core fragment comprises a sequence of Formula B:
[L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ,
wherein:
x=1 and y=0 or 1;
one of [ASt Domain1], [ACH Domain], and [ASt Domain2] comprises a nucleotide having a non-naturally occurring modification; and
the TREM retains the ability to: support protein synthesis; be able to be charged by a synthetase, be bound by an elongation factor, introduce an amino acid into a peptide chain, support elongation, or support initiation.

78. The method of any one of the preceding embodiments, wherein the TREM fragment comprises a portion of a TREM, wherein the TREM comprises a sequence of Formula A:
[L1]-[ASt Domain1]-[L2]-[DH Domain]-[L3]-[ACH Domain]-[VL Domain]-[TH Domain]-[L4]-[ASt Domain2], and wherein:
the TREM fragment comprises:
a non-naturally occurring modification; and
one, two, three or all or any combination of the following:
(a) a TREM half (e.g., from a cleavage in the ACH Domain, e.g., in the anticodon sequence, e.g., a 5′ half or a 3′ half);
(b) a 5′ fragment (e.g., a fragment comprising the 5′ end, e.g., from a cleavage in a DH Domain or the ACH Domain);
(c) a 3′ fragment (e.g., a fragment comprising the 3′ end, e.g., from a cleavage in the TH Domain); or
(d) an internal fragment (e.g., from a cleavage in any one of the ACH Domain, DH Domain or TH Domain).

79. The method of any one of claims 75-78, wherein the TREM Domain comprises a plurality of nucleotides each having a non-naturally occurring modification.

80. The method of any one of claims 75-79, wherein the non-naturally occurring modification is a modification in a base or a backbone of a nucleotide, e.g., a modification chosen from any one of Tables 5, 6, 7, 8 or 9.

81. The method of any one of claims 75-80, wherein the TREM, TREM core fragment or TREM fragment recognizes a codon provided in Table 2 or Table 3.

82. The method of any one of claims 75-80, wherein the TREM, TREM core fragment or TREM fragment is a cognate TREM.

83. The method of any one of claims 75-80, wherein the TREM, TREM core fragment or TREM fragment is a non-cognate TREM.

84. The method of any one of claims 75-80, wherein the TREM, TREM core fragment or TREM fragment is encoded by a sequence provided in Table 4, e.g., any one of SEQ ID NOs 1-451.

85. The method of any one of claims 75-80, wherein the TREM, TREM core fragment or TREM fragment is encoded by a consensus sequence chosen from any one of SEQ ID NOs: 562-621.

86. A pharmaceutical composition comprising a TREM, TREM core fragment or TREM fragment described herein.

87. A method of making a TREM, TREM core fragment or TREM fragment, comprising linking a first nucleotide to a second nucleotide to form the TREM.

88. The method of claim 87, wherein the TREM, TREM core fragment or TREM fragment is synthetic.

89. The method of claim 88, wherein the TREM, TREM core fragment or TREM fragment is made by cell-free solid phase synthesis.
